SEQUENCE LISTING (219 sequences)

SEQ ID NO: 1 - protein, 12 aa, synthetic construct
  REGION 1..12: note = Spacer (IgG4 hinge)
ESKYGPPCPPCP

SEQ ID NO: 2 - DNA, 36 bp, synthetic construct
  misc_feature 1..36: note = Spacer (IgG4 hinge)
  source 1..36: mol_type = other DNA
gaatctaagtacggaccgccctgccccccttgccct

SEQ ID NO: 3 - protein, 119 aa, synthetic construct
  REGION 1..119: note = Hinge-CH3 spacer
ESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 4 - DNA, 357 bp, synthetic construct
  misc_feature 1..357: note = Hinge-CH3 spacer
  source 1..357: mol_type = other DNA
gaatctaagtacggaccgccttgtcctccatgtcctggccagccaagagaaccccaggtgtacacactgcctccaagccaagaggaaatgaccaagaaccaggtgtccctgacctgcctggtcaagggcttctacccttccgatatcgccgtggaatgggagagcaatggccagcctgagaacaactacaagaccacacctcctgtgctggacagcgacggctcattcttcctgtacagccggctgaccgtggacaagagcagatggcaagagggcaacgtgttcagctgcagcgtgatgcacgaggccctgcacaaccactacacccagaagtctctgagcctgagcctgggcaag

SEQ ID NO: 5 - protein, 282 aa, synthetic construct
  REGION 1..282: note = IgD-hinge-Fc
RWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDH

SEQ ID NO: 6 - protein, 24 aa, synthetic construct
  REGION 1..24: note = T2A
LEGGGEGRGSLLTCGDVEENPGPR

SEQ ID NO: 7 - protein, 357 aa, synthetic construct
  REGION 1..357: note = tEGFR
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 8 - protein, 27 aa, Homo sapiens
  REGION 1..27: note = MISC_FEATURE - CD28
FWVLVVVGGVLACYSLLVTVAFIIFWV

SEQ ID NO: 9 - protein, 66 aa, Homo sapiens
  REGION 1..66: note = MISC_FEATURE - CD28
IEVMYPPPYLDNEKSNGTIIHVKGKHLCPSPLFPGPSKPFWVLVVVGGVLACYSLLVTVAFIIFWV

SEQ ID NO: 10 - protein, 41 aa, Homo sapiens
  REGION 1..41: note = MISC_FEATURE - CD28
RSKRSRLLHSDYMNMTPRRPGPTRKHYQPYAPPRDFAAYRS

SEQ ID NO: 11 - protein, 41 aa, Homo sapiens
  REGION 1..41: note = MISC_FEATURE - CD28 (LL to GG)
RSKRSRGGHSDYMNMTPRRPGPTRKHYQPYAPPRDFAAYRS

SEQ ID NO: 12 - protein, 42 aa, Homo sapiens
  REGION 1..42: note = MISC_FEATURE - 4-1BB
KRGRKKLLYIFKQPFMRPVQTTQEEDGCSCRFPEEEEGGCEL

SEQ ID NO: 13 - protein, 112 aa, Homo sapiens
  REGION 1..112: note = MISC_FEATURE - CD3 zeta
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 14 - protein, 112 aa, Homo sapiens
  REGION 1..112: note = MISC_FEATURE - CD3 zeta
RVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - protein, 112 aa, Homo sapiens
  REGION 1..112: note = MISC_FEATURE - CD3 zeta
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 16 - protein, 335 aa, synthetic construct
  REGION 1..335: note = tEGFR
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 17 - protein, 18 aa, synthetic construct
  REGION 1..18: note = T2A
EGRGSLLTCGDVEENPGP

SEQ ID NO: 18 - protein, 22 aa, synthetic construct
  REGION 1..22: note = P2A
GSGATNFSLLKQAGDVEENPGP

SEQ ID NO: 19 - protein, 19 aa, synthetic construct
  REGION 1..19: note = P2A
ATNFSLLKQAGDVEENPGP

SEQ ID NO: 20 - protein, 20 aa, synthetic construct
  REGION 1..20: note = E2A
QCTNYALLKLAGDVESNPGP

SEQ ID NO: 21 - protein, 22 aa, synthetic construct
  REGION 1..22: note = F2A
VKQTLNFDLLKLAGDVESNPGP

SEQ ID NO: 22 - protein, 10 aa, synthetic construct
  REGION 1..10: note = linker
  REPEAT 5..9: note = SGGGG is repeated 5 times
PGGGSGGGGP

SEQ ID NO: 23 - protein, 17 aa, synthetic construct
  REGION 1..17: note = linker
GSADDAKKDAAKKDGKS

SEQ ID NO: 24 - protein, 18 aa, synthetic construct
  REGION 1..18: note = linker
GSTSGSGKPGSGEGSTKG

SEQ ID NO: 25 - skipped

SEQ ID NO: 26 - protein, 15 aa, synthetic construct
  REGION 1..15: note = hinge
EPKSCDKTHTCPPCP

SEQ ID NO: 27 - protein, 12 aa, synthetic construct
  REGION 1..12: note = hinge
ERKCCVECPPCP

SEQ ID NO: 28 - protein, 61 aa, synthetic construct
  REGION 1..61: note = hinge
ELKTPLGDTHTCPRCPEPKSCDTPPPCPRCPEPKSCDTPPPCPRCPEPKSCDTPPPCPRCP

SEQ ID NO: 29 - protein, 12 aa, synthetic construct
  REGION 1..12: note = hinge
ESKYGPPCPSCP

SEQ ID NO: 30 - DNA, 36 bp, synthetic construct
  misc_feature 1..36: note = Spacer (IgG4hinge) O/SSE
  source 1..36: mol_type = other DNA
gagtctaaatacggaccgccttgtcctccttgtccc

SEQ ID NO: 31 - protein, 9 aa, synthetic construct
  REGION 1..9: note = hinge
YGPPCPPCP

SEQ ID NO: 32 - protein, 10 aa, synthetic construct
  REGION 1..10: note = hinge
KYGPPCPPCP

SEQ ID NO: 33 - protein, 14 aa, synthetic construct
  REGION 1..14: note = hinge
EVVVKYGPPCPPCP

SEQ ID NO: 34 - protein, 228 aa, synthetic construct
  REGION 1..228: note = Hinge-CH2-CH3 spacer
ESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 35 - DNA, 684 bp, synthetic construct
  misc_feature 1..684: note = Hinge-CH2-CH3 spacer
  source 1..684: mol_type = other DNA
gaatctaagtacggaccgccttgtcctccatgtcctgctcctccagttgccggaccttccgtgttcctgtttcctccaaagcctaaggacaccctgatgatcagcagaacccctgaagtgacctgcgtggtggtggacgtgtcccaagaggatcctgaggtgcagttcaactggtatgtggacggcgtggaagtgcacaacgccaagaccaagcctagagaggaacagttccagagcacctacagagtggtgtccgtgctgacagtgctgcaccaggattggctgaacggcaaagagtacaagtgcaaggtgtccaacaagggcctgcctagcagcatcgagaaaaccatcagcaaggccaagggccagccaagagaaccccaggtgtacacactgcctccaagccaagaggaaatgaccaagaaccaggtgtccctgacctgcctggtcaagggcttctacccttccgatatcgccgtggaatgggagagcaatggccagcctgagaacaactacaagaccacacctcctgtgctggacagcgacggctcattcttcctgtacagccggctgaccgtggacaagagcagatggcaagagggcaacgtgttcagctgcagcgtgatgcacgaggccctgcacaaccactacacccagaagtctctgagcctgagcctgggcaag

SEQ ID NO: 36 - protein, 229 aa, synthetic construct
  REGION 1..229: note = Hinge-CH2-CH3 spacer
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 37 - protein, 228 aa, synthetic construct; identical to SEQ ID NO: 34
  REGION 1..228: note = IgG4/IgG2 hinge- IgG2/IgG4 CH2- IgG4 CH3 spacer

SEQ ID NO: 38 - DNA, 684 bp, synthetic construct
  misc_feature 1..684: note = IgG4/IgG2 hinge- IgG2/IgG4 CH2- IgG4 CH3 spacer
  source 1..684: mol_type = other DNA
gaatctaagtacggaccgccctgccctccctgccctgctcctcctgtggctggaccaagcgtgttcctgtttccacctaagcctaaagataccctgatgatttcccgcacacctgaagtgacttgcgtggtcgtggacgtgagccaggaggatccagaagtgcagttcaactggtacgtggacggcgtggaagtccacaatgctaagactaaaccccgagaggaacagtttcagtcaacttaccgggtcgtgagcgtgctgaccgtcctgcatcaggattggctgaacgggaaggagtataagtgcaaagtgtctaataagggactgcctagctccatcgagaaaacaattagtaaggcaaaagggcagcctcgagaaccacaggtgtataccctgccccctagccaggaggaaatgaccaagaaccaggtgtccctgacatgtctggtcaaaggcttctatccaagtgacatcgccgtggagtgggaatcaaatgggcagcccgagaacaattacaagaccacaccacccgtgctggactctgatggaagtttctttctgtattccaggctgaccgtggataaatctcgctggcaggagggcaacgtgttctcttgcagtgtcatgcacgaagccctgcacaatcattatacacagaagtcactgagcctgtccctgggcaaa

SEQ ID NO: 39 - protein, 5 aa, synthetic construct
  REGION 1..5: note = 4GS linker
GGGGS

SEQ ID NO: 40 - protein, 4 aa, synthetic construct
  REGION 1..4: note = 3GS linker
GGGS

SEQ ID NO: 41 - protein, 15 aa, synthetic construct
  REGION 1..15: note = (4GS)3 linker
GGGGSGGGGSGGGGS

SEQ ID NO: 42 - protein, 16 aa, synthetic construct
  REGION 1..16: note = CD33 Signal Peptide
MPLLLLLPLLWAGALA

SEQ ID NO: 43 - protein, 20 aa, synthetic construct
  REGION 1..20: note = human IgG-kappa signal peptide
MVLQTQVFISLLLWISGAYG

SEQ ID NO: 44 - DNA, 60 bp, synthetic construct
  misc_feature 1..60: note = human IgG-kappa signal sequence
  source 1..60: mol_type = other DNA
atggtgctgcagacccaggtgttcatcagcctgctgctgtggatctccggagcatacgga

SEQ ID NO: 45 - protein, 22 aa, synthetic construct
  REGION 1..22: note = GMCSFR alpha chain signal peptide
MLLLVTSLLLCELPHPAFLLIP

SEQ ID NO: 46 - DNA, 66 bp, synthetic construct
  misc_feature 1..66: note = GMCSFR alpha chain signal sequence
  source 1..66: mol_type = other DNA
atgcttctcctggtgacaagccttctgctctgtgagttaccacacccagcattcctcctgatccca

SEQ ID NO: 47 - protein, 18 aa, synthetic construct
  REGION 1..18: note = CD8 alpha signal peptide
MALPVTALLLPLALLLHA

SEQ ID NO: 48 - protein, 326 aa, Homo sapiens
  REGION 1..326: note = MISC_FEATURE - IgG2 Fc (Uniprot P01859)
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 49 - protein, 327 aa, Homo sapiens
  REGION 1..327: note = MISC_FEATURE - IgG4 Fc (Uniprot P01861)
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 50 - protein, 7 aa, synthetic construct
  REGION 1..7: note = CDR-H1
GYTFTSY

SEQ ID NO: 51 - protein, 10 aa, synthetic construct
  REGION 1..10: note = CDR-H1
GYTFTSYGIS

SEQ ID NO: 52 - protein, 5 aa, synthetic construct
  REGION 1..5: note = CDR-H1
SYGIS

SEQ ID NO: 53 - protein, 8 aa, synthetic construct
  REGION 1..8: note = CDR-H1
GYTFTSYG

SEQ ID NO: 54 - protein, 6 aa, synthetic construct
  REGION 1..6: note = CDR-H2
SAYNGN

SEQ ID NO: 55 - protein, 10 aa, synthetic construct
  REGION 1..10: note = CDR-H2
WISAYNGNTK

SEQ ID NO: 56 - protein, 17 aa, synthetic construct
  REGION 1..17: note = CDR-H2
WISAYNGNTKYAQKLQG

SEQ ID NO: 57 - protein, 8 aa, synthetic construct
  REGION 1..8: note = CDR-H2
ISAYNGNT

SEQ ID NO: 58 - protein, 15 aa, synthetic construct
  REGION 1..15: note = CDR-H3
DEDILTGYNYYGMDV

SEQ ID NO: 59 - protein, 17 aa, synthetic construct
  REGION 1..17: note = CDR-H3
ARDEDILTGYNYYGMDV

SEQ ID NO: 60 - protein, 12 aa, synthetic construct
  REGION 1..12: note = CDR-L1
TLSSGHSSYAIA

SEQ ID NO: 61 - protein, 7 aa, synthetic construct
  REGION 1..7: note = CDR-L1
SGHSSYA

SEQ ID NO: 62 - protein, 11 aa, synthetic construct
  REGION 1..11: note = CDR-L2
LNSDGSHSKGD

SEQ ID NO: 63 - protein, 7 aa, synthetic construct
  REGION 1..7: note = CDR-L2
LNSDGSH

SEQ ID NO: 64 - protein, 9 aa, synthetic construct
  REGION 1..9: note = CDR-L3
QTWGTGIRV

SEQ ID NO: 65 - protein, 7 aa, synthetic construct
  REGION 1..7: note = CDR-H1
GGSISNY

SEQ ID NO: 66 - protein, 10 aa, synthetic construct
  REGION 1..10: note = CDR-H1
GGSISNYYWS

SEQ ID NO: 67 - protein, 5 aa, synthetic construct
  REGION 1..5: note = CDR-H1
NYYWS

SEQ ID NO: 68 - protein, 8 aa, synthetic construct
  REGION 1..8: note = CDR-H1
GGSISNYY

SEQ ID NO: 69 - protein, 5 aa, synthetic construct
  REGION 1..5: note = CDR-H2
YTSGS

SEQ ID NO: 70 - protein, 9 aa, synthetic construct
  REGION 1..9: note = CDR-H2
RIYTSGSTN

SEQ ID NO: 71 - protein, 16 aa, synthetic construct
  REGION 1..16: note = CDR-H2
RIYTSGSTNYNPSLKS

SEQ ID NO: 72 - protein, 7 aa, synthetic construct
  REGION 1..7: note = CDR-H2
IYTSGST

SEQ ID NO: 73 - protein, 11 aa, synthetic construct
  REGION 1..11: note = CDR-H3
YYDILTGFFDY

SEQ ID NO: 74 - protein, 13 aa, synthetic construct
  REGION 1..13: note = CDR-H3
ARYYDILTGFFDY

SEQ ID NO: 75 - protein, 11 aa, synthetic construct
  REGION 1..11: note = CDR-L1
RMSQDISSYLA

SEQ ID NO: 76 - protein, 6 aa, synthetic construct
  REGION 1..6: note = CDR-L1
QDISSY

SEQ ID NO: 77 - protein, 7 aa, synthetic construct
  REGION 1..7: note = CDR-L2
AASSLQS

SEQ ID NO: 78 - skipped

SEQ ID NO: 79 - protein, 9 aa, synthetic construct
  REGION 1..9: note = CDR-L3
QQYDSFPPT

SEQ ID NO: 80 - protein, 9 aa, synthetic construct
  REGION 1..9: note = CDR-H1
GGSINSTTS

SEQ ID NO: 81 - protein, 12 aa, synthetic construct
  REGION 1..12: note = CDR-H1
GGSINSTTSYWA

SEQ ID NO: 82 - protein, 7 aa, synthetic construct
  REGION 1..7: note = CDR-H1
STTSYWA

SEQ ID NO: 83 - protein, 10 aa, synthetic construct
  REGION 1..10: note = CDR-H1
GGSINSTTSY

SEQ ID NO: 84 - protein, 4 aa, synthetic construct
  REGION 1..4: note = CDR-H2
YSGK

SEQ ID NO: 85 - protein, 9 aa, synthetic construct
  REGION 1..9: note = CDR-H2
TIFYSGKTY

SEQ ID NO: 86 - protein, 16 aa, synthetic construct
  REGION 1..16: note = CDR-H2
TIFYSGKTYNNPSLKS

SEQ ID NO: 87 - protein, 7 aa, synthetic construct
  REGION 1..7: note = CDR-H2
IFYSGKT

SEQ ID NO: 88 - protein, 11 aa, synthetic construct
  REGION 1..11: note = CDR-H3
FDYGFHDAFDI

SEQ ID NO: 89 - protein, 13 aa, synthetic construct
  REGION 1..13: note = CDR-H3
ARFDYGFHDAFDI

SEQ ID NO: 90 - protein, 12 aa, synthetic construct
  REGION 1..12: note = CDR-L1
RASQSITSDYLS

SEQ ID NO: 91 - protein, 7 aa, synthetic construct
  REGION 1..7: note = CDR-L1
QSITSDY

SEQ ID NO: 92 - protein, 7 aa, synthetic construct
  REGION 1..7: note = CDR-L2
GASTRAT

SEQ ID NO: 93 - skipped

SEQ ID NO: 94 - protein, 8 aa, synthetic construct
  REGION 1..8: note = CDR-L3
QQDYNLYT

SEQ ID NO: 95 - protein, 6 aa, synthetic construct
  REGION 1..6: note = CDR-H2
SAYTGN

SEQ ID NO: 96 - protein, 10 aa, synthetic construct
  REGION 1..10: note = CDR-H2
WISAYTGNTR

SEQ ID NO: 97 - protein, 17 aa, synthetic construct
  REGION 1..17: note = CDR-H2
WISAYTGNTRYAQKLQG

SEQ ID NO: 98 - protein, 8 aa, synthetic construct
  REGION 1..8: note = CDR-H2
ISAYTGNT

SEQ ID NO: 99 - protein, 15 aa, synthetic construct
  REGION 1..15: note = CDR-H3
EEGATTDYDYYGMDV

SEQ ID NO: 100 - protein, 17 aa, synthetic construct
  REGION 1..17: note = CDR-H3
AREEGATTDYDYYGMDV

SEQ ID NO: 101 - DNA, 372 bp, synthetic construct
  misc_feature 1..372: note = VH
  source 1..372: mol_type = other DNA
caggttcagctggtgcagtctggagctgaggtgaagaagcctggggcctcagtgaaggtctcctgcaaggcttctggttacacctttaccagctatggtatcagctgggtgcgacaggcccctggacaagggcttgagtggatgggatggatcagcgcttacaatggtaacacaaagtatgcacagaagctccagggcagagtcaccatgaccacagacacatccacgagcacagcctacatggagctgaggagcctgagatctgacgacacggccgtgtattactgtgcgagagatgaggatattttgactggttacaactactacggtatggacgtctggggccaagggaccacggtcaccgtctcctca

SEQ ID NO: 102 - DNA, 372 bp, synthetic construct
  misc_feature 1..372: note = VH O/SSE
  source 1..372: mol_type = other DNA
caggtgcagctggttcaatctggcgccgaagtgaagaaaccaggcgcctctgtgaaggtgtcctgcaaggccagcggctacacctttaccagctacggcatcagctgggtccgacaggctcctggacaaggcttggaatggatgggctggatcagcgcctacaacggcaacaccaaatacgcccagaaactgcagggcagagtgaccatgaccaccgacaccagcacaagcaccgcctacatggaactgcggagcctgagatccgatgacaccgccgtgtactactgcgccagagatgaggacatcctgaccggctacaactactacggcatggacgtgtggggccagggcacaacagtgacagtttcttct

SEQ ID NO: 103 - protein, 124 aa, synthetic construct
  REGION 1..124: note = VH
QVQLVQSGAEVKKPGASVKVSCKASGYTFTSYGISWVRQAPGQGLEWMGWISAYNGNTKYAQKLQGRVTMTTDTSTSTAYMELRSLRSDDTAVYYCARDEDILTGYNYYGMDVWGQGTTVTVSS

SEQ ID NO: 104 - DNA, 336 bp, synthetic construct
  misc_feature 1..336: note = VL
  source 1..336: mol_type = other DNA
cagcttgtgctgactcaatcgccctctgcctctgcctccctgggagcctcggtcaagctcacctgcactctgagcagtgggcacagcagctacgccatcgcatggcatcagcagcagccagagaagggccctcggtacttgatgaagcttaacagtgatggcagccacagcaagggggacgggatccctgatcgcttctcaggctccagctctggggctgagcgctacctcaccatctccagcctccagtctgaggatgaggctgactattactgtcagacctggggcactggcattcgggtgttcggtggaggaaccaaactgactgtcctaggc

SEQ ID NO: 105 - DNA, 336 bp, synthetic construct
  misc_feature 1..336: note = VL O/SSE
  source 1..336: mol_type = other DNA
caactggtgctgacacagtctcctagcgcctctgcttctctgggagccagcgtgaagctgacctgtacactgtctagcggccacagcagctacgccattgcttggcatcagcagcagcccgagaagggccctagatacctgatgaagctgaacagcgacggcagccactctaaaggcgacggcatccccgatagattcagcggcagttctagcggagccgagcgctacctgacaatcagctctctgcaatccgaggacgaggccgactactactgtcagacatggggcaccggcatcagagtgtttggcggaggcaccaagctgacagtgcttgga

SEQ ID NO: 106 - protein, 112 aa, synthetic construct
  REGION 1..112: note = VL
QLVLTQSPSASASLGASVKLTCTLSSGHSSYAIAWHQQQPEKGPRYLMKLNSDGSHSKGDGIPDRFSGSSSGAERYLTISSLQSEDEADYYCQTWGTGIRVFGGGTKLTVLG

SEQ ID NO: 107 - DNA, 753 bp, synthetic construct
  misc_feature 1..753: note = scFv (VH-VL)
  source 1..753: mol_type = other DNA
caggttcagctggtgcagtctggagctgaggtgaagaagcctggggcctcagtgaaggtctcctgcaaggcttctggttacacctttaccagctatggtatcagctgggtgcgacaggcccctggacaagggcttgagtggatgggatggatcagcgcttacaatggtaacacaaagtatgcacagaagctccagggcagagtcaccatgaccacagacacatccacgagcacagcctacatggagctgaggagcctgagatctgacgacacggccgtgtattactgtgcgagagatgaggatattttgactggttacaactactacggtatggacgtctggggccaagggaccacggtcaccgtctcctcaggtggtggtggtagcggcggcggcggctctggtggtggtggatcccagcttgtgctgactcaatcgccctctgcctctgcctccctgggagcctcggtcaagctcacctgcactctgagcagtgggcacagcagctacgccatcgcatggcatcagcagcagccagagaagggccctcggtacttgatgaagcttaacagtgatggcagccacagcaagggggacgggatccctgatcgcttctcaggctccagctctggggctgagcgctacctcaccatctccagcctccagtctgaggatgaggctgactattactgtcagacctggggcactggcattcgggtgttcggtggaggaaccaaactgactgtcctaggc

SEQ ID NO: 108 - DNA, 753 bp, synthetic construct
  misc_feature 1..753: note = scFv (VH-VL) O/SSE
  source 1..753: mol_type = other DNA
caggtgcagctggttcaatctggcgccgaagtgaagaaaccaggcgcctctgtgaaggtgtcctgcaaggccagcggctacacctttaccagctacggcatcagctgggtccgacaggctcctggacaaggcttggaatggatgggctggatcagcgcctacaacggcaacaccaaatacgcccagaaactgcagggcagagtgaccatgaccaccgacaccagcacaagcaccgcctacatggaactgcggagcctgagatccgatgacaccgccgtgtactactgcgccagagatgaggacatcctgaccggctacaactactacggcatggacgtgtggggccagggcacaacagtgacagtttcttctggcggcggaggatctggcggaggtggaagcggaggcggtggatctcaactggtgctgacacagtctcctagcgcctctgcttctctgggagccagcgtgaagctgacctgtacactgtctagcggccacagcagctacgccattgcttggcatcagcagcagcccgagaagggccctagatacctgatgaagctgaacagcgacggcagccactctaaaggcgacggcatccccgatagattcagcggcagttctagcggagccgagcgctacctgacaatcagctctctgcaatccgaggacgaggccgactactactgtcagacatggggcaccggcatcagagtgtttggcggaggcaccaagctgacagtgcttgga

SEQ ID NO: 109 - protein, 251 aa, synthetic construct
  REGION 1..251: note = scFv (VH-VL)
QVQLVQSGAEVKKPGASVKVSCKASGYTFTSYGISWVRQAPGQGLEWMGWISAYNGNTKYAQKLQGRVTMTTDTSTSTAYMELRSLRSDDTAVYYCARDEDILTGYNYYGMDVWGQGTTVTVSSGGGGSGGGGSGGGGSQLVLTQSPSASASLGASVKLTCTLSSGHSSYAIAWHQQQPEKGPRYLMKLNSDGSHSKGDGIPDRFSGSSSGAERYLTISSLQSEDEADYYCQTWGTGIRVFGGGTKLTVLG

SEQ ID NO: 110 - DNA, 357 bp, synthetic construct
  misc_feature 1..357: note = VH
  source 1..357: mol_type = other DNA
caggtgcagctgcaggagtcgggcccaggactggtgaagccttcggagaccctgtccctcacctgcactgtctctggaggctccatcagtaattactactggagctggatccggcagcccgccgggaagggactggagtggattgggcgtatctataccagtgggagcaccaactacaacccctccctcaagagtcgagtcaccatgtcagtagacacgtccaagaaccagttctccctgaagctgagttctttgaccgccgcggacacggccatatattactgtgcgaggtattacgatattttgactggtttctttgactactggggccagggaaccctggtcaccgtctcctca

SEQ ID NO: 111 - DNA, 357 bp, synthetic construct
  misc_feature 1..357: note = VH O/SSE
  source 1..357: mol_type = other DNA
caggttcagctgcaagagtctggccctggcctggtcaagcctagcgaaacactgagcctgacctgtaccgtgtctggcggcagcatctccaactactactggtcctggatcagacagcctgccggcaaaggcctggaatggatcggcagaatctacaccagcggcagcaccaactacaaccccagcctgaagtccagagtgaccatgagcgtggacaccagcaagaaccagttctccctgaagctgagcagcctgacagccgccgataccgccatctactactgtgcccggtactacgatatcctgaccggcttcttcgactactggggccagggaacactggtcacagtttctagc

SEQ ID NO: 112 - protein, 119 aa, synthetic construct
  REGION 1..119: note = VH
QVQLQESGPGLVKPSETLSLTCTVSGGSISNYYWSWIRQPAGKGLEWIGRIYTSGSTNYNPSLKSRVTMSVDTSKNQFSLKLSSLTAADTAIYYCARYYDILTGFFDYWGQGTLVTVSS

SEQ ID NO: 113 - DNA, 324 bp, synthetic construct
  misc_feature 1..324: note = VL
  source 1..324: mol_type = other DNA
gtcatctggatgacccagtctccatccttactctctgcatctacaggagacagtgtcaccatcagttgtcggatgagtcaggacattagcagttatttagcctggtatcagcaaaaaccagggaaagcccctgagctcctgatctatgctgcatccagtttgcaaagtggggtcccatcaaggttcagtggcagtggatctgggacagacttcactctcaccatcagttccctgcagtctgaagattttgctacttattactgtcaacagtatgatagtttccctccgacgttcggccaagggaccaaggtggaattcaaacgg

SEQ ID NO: 114 - DNA, 324 bp, synthetic construct
  misc_feature 1..324: note = VL O/SSE
  source 1..324: mol_type = other DNA
gtgatttggatgacacagagccctagcctgctgagcgccagcacaggcgatagcgtgaccatcagctgcagaatgagccaggacatcagcagctacctggcttggtatcagcagaagcctggcaaggcccctgaactgctgatctatgccgcttccagtctgcagagcggcgtgccatctagattttccggcagcggctctggcaccgacttcaccctgacaatcagctccctgcagtccgaggacttcgccacctactattgccagcagtacgacagcttccctccaacctttggccagggcaccaaggtggaattcaagcgc

SEQ ID NO: 115 - protein, 108 aa, synthetic construct
  REGION 1..108: note = VL
VIWMTQSPSLLSASTGDSVTISCRMSQDISSYLAWYQQKPGKAPELLIYAASSLQSGVPSRFSGSGSGTDFTLTISSLQSEDFATYYCQQYDSFPPTFGQGTKVEFKR

SEQ ID NO: 116 - DNA, 726 bp, synthetic construct
  misc_feature 1..726: note = scFv (VH-VL)
  source 1..726: mol_type = other DNA
caggtgcagctgcaggagtcgggcccaggactggtgaagccttcggagaccctgtccctcacctgcactgtctctggaggctccatcagtaattactactggagctggatccggcagcccgccgggaagggactggagtggattgggcgtatctataccagtgggagcaccaactacaacccctccctcaagagtcgagtcaccatgtcagtagacacgtccaagaaccagttctccctgaagctgagttctttgaccgccgcggacacggccatatattactgtgcgaggtattacgatattttgactggtttctttgactactggggccagggaaccctggtcaccgtctcctcaggtggtggtggtagcggcggcggcggctctggtggtggtggatccgtcatctggatgacccagtctccatccttactctctgcatctacaggagacagtgtcaccatcagttgtcggatgagtcaggacattagcagttatttagcctggtatcagcaaaaaccagggaaagcccctgagctcctgatctatgctgcatccagtttgcaaagtggggtcccatcaaggttcagtggcagtggatctgggacagacttcactctcaccatcagttccctgcagtctgaagattttgctacttattactgtcaacagtatgatagtttccctccgacgttcggccaagggaccaaggtggaattcaaacgg

SEQ ID NO: 117 - DNA, 726 bp, synthetic construct
  misc_feature 1..726: note = scFv (VH-VL) O/SSE
  source 1..726: mol_type = other DNA
caggttcagctgcaagagtctggccctggcctggtcaagcctagcgaaacactgagcctgacctgtaccgtgtctggcggcagcatctccaactactactggtcctggatcagacagcctgccggcaaaggcctggaatggatcggcagaatctacaccagcggcagcaccaactacaaccccagcctgaagtccagagtgaccatgagcgtggacaccagcaagaaccagttctccctgaagctgagcagcctgacagccgccgataccgccatctactactgtgcccggtactacgatatcctgaccggcttcttcgactactggggccagggaacactggtcacagtttctagcggaggcggaggatctggtggcggaggaagtggcggaggcggttctgtgatttggatgacacagagccctagcctgctgagcgccagcacaggcgatagcgtgaccatcagctgcagaatgagccaggacatcagcagctacctggcttggtatcagcagaagcctggcaaggcccctgaactgctgatctatgccgcttccagtctgcagagcggcgtgccatctagattttccggcagcggctctggcaccgacttcaccctgacaatcagctccctgcagtccgaggacttcgccacctactattgccagcagtacgacagcttccctccaacctttggccagggcaccaaggtggaattcaagcgc

SEQ ID NO: 118 - protein, 242 aa, synthetic construct
  REGION 1..242: note = scFv (VH-VL)
QVQLQESGPGLVKPSETLSLTCTVSGGSISNYYWSWIRQPAGKGLEWIGRIYTSGSTNYNPSLKSRVTMSVDTSKNQFSLKLSSLTAADTAIYYCARYYDILTGFFDYWGQGTLVTVSSGGGGSGGGGSGGGGSVIWMTQSPSLLSASTGDSVTISCRMSQDISSYLAWYQQKPGKAPELLIYAASSLQSGVPSRFSGSGSGTDFTLTISSLQSEDFATYYCQQYDSFPPTFGQGTKVEFKR

SEQ ID NO: 119 - DNA, 363 bp, synthetic construct
  misc_feature 1..363: note = VH
  source 1..363: mol_type = other DNA
cagctgcagctgcaggagtcgggcccaggactggtgaagccttcggagaccctgtccctcacctgcactgtctctggtggctccatcaacagtactacttcctactgggcctggatccgccagcccccagggaaggggctggagtggattgggactatcttttatagtgggaaaacctacaacaacccgtccctcaagagtcgagtcaccatgtccgtagacacgtccaagaaccacttctccctgaaggtgaactctgtgaccgccgcagacacggctgtgtattactgtgcgaggtttgactacggttttcatgatgcttttgatatctggggccaggggacaatggtcaccgtctcttca

SEQ ID NO: 120 - DNA, 363 bp, synthetic construct
  misc_feature 1..363: note = VH O/SSE
  source 1..363: mol_type = other DNA
cagctccagctgcaagaatctggacctggcctggtcaagcccagcgagacactgtctctgacctgtacagtgtccggcggcagcatcaatagcaccacaagctactgggcctggatcagacagcctcctggcaaaggcctggaatggatcggcaccatcttctacagcggcaagacctacaacaaccccagcctgaagtccagagtgaccatgagcgtggacaccagcaagaaccacttcagcctgaaagtgaacagcgtgacagccgccgataccgccgtgtactactgcgccagattcgactacggcttccacgacgccttcgacatctggggccagggcacaatggtcacagtttctagc

SEQ ID NO: 121 - protein, 121 aa, synthetic construct
  REGION 1..121: note = VH
QLQLQESGPGLVKPSETLSLTCTVSGGSINSTTSYWAWIRQPPGKGLEWIGTIFYSGKTYNNPSLKSRVTMSVDTSKNHFSLKVNSVTAADTAVYYCARFDYGFHDAFDIWGQGTMVTVSS

SEQ ID NO: 122 - DNA, 324 bp, synthetic construct
  misc_feature 1..324: note = VL
  source 1..324: mol_type = other DNA
gaaattgtaatgacacagtctccagccaccctgtctttgtctccaggggaaagagccaccctctcctgcagggccagtcagagtattaccagcgactacttatcctggtaccaacaaaaacctgggcaggctcccaggctcctcatctatggtgcatccaccagggccactggcatcccagccaggttcagtggcagtgggtctgggacagacttcactctcaccatcagcagcctgcagcctgaagattttgtagtttattactgtcagcaggattataacttgtacacttttggccaggggaccaagctggagatcaaacgg

SEQ ID NO: 123 - DNA, 324 bp, synthetic construct
  misc_feature 1..324: note = VL O/SSE
  source 1..324: mol_type = other DNA
gagattgtgatgacacagagccccgccactctgagccttagtcctggcgaaagagccacactgagctgcagagccagccagagcatcaccagcgattacctgagctggtatcagcagaagcccggacaggctcccagactgctgatctatggcgcctctacaagagccaccggcattcccgcccgcttttctggctctggaagcggcaccgacttcaccctgaccatatctagcctgcagcctgaggacttcgtggtgtactattgccagcaggactacaacctgtacaccttcggccaggggaccaagctggaaatcaagaga

SEQ ID NO: 124 - protein, 108 aa, synthetic construct
  REGION 1..108: note = VL
EIVMTQSPATLSLSPGERATLSCRASQSITSDYLSWYQQKPGQAPRLLIYGASTRATGIPARFSGSGSGTDFTLTISSLQPEDFVVYYCQQDYNLYTFGQGTKLEIKR

SEQ ID NO: 125 - DNA, 732 bp, synthetic construct
  misc_feature 1..732: note = scFv (VH-VL)
  source 1..732: mol_type = other DNA
cagctgcagctgcaggagtcgggcccaggactggtgaagccttcggagaccctgtccctcacctgcactgtctctggtggctccatcaacagtactacttcctactgggcctggatccgccagcccccagggaaggggctggagtggattgggactatcttttatagtgggaaaacctacaacaacccgtccctcaagagtcgagtcaccatgtccgtagacacgtccaagaaccacttctccctgaaggtgaactctgtgaccgccgcagacacggctgtgtattactgtgcgaggtttgactacggttttcatgatgcttttgatatctggggccaggggacaatggtcaccgtctcttcaggtggtggtggtagcggcggcggcggctctggtggtggtggatccgaaattgtaatgacacagtctccagccaccctgtctttgtctccaggggaaagagccaccctctcctgcagggccagtcagagtattaccagcgactacttatcctggtaccaacaaaaacctgggcaggctcccaggctcctcatctatggtgcatccaccagggccactggcatcccagccaggttcagtggcagtgggtctgggacagacttcactctcaccatcagcagcctgcagcctgaagattttgtagtttattactgtcagcaggattataacttgtacacttttggccaggggaccaagctggagatcaaacgg

SEQ ID NO: 126 - DNA, 732 bp, synthetic construct
  misc_feature 1..732: note = scFv (VH-VL) O/SSE
  source 1..732: mol_type = other DNA
cagctccagctgcaagaatctggacctggcctggtcaagcccagcgagacactgtctctgacctgtacagtgtccggcggcagcatcaatagcaccacaagctactgggcctggatcagacagcctcctggcaaaggcctggaatggatcggcaccatcttctacagcggcaagacctacaacaaccccagcctgaagtccagagtgaccatgagcgtggacaccagcaagaaccacttcagcctgaaagtgaacagcgtgacagccgccgataccgccgtgtactactgcgccagattcgactacggcttccacgacgccttcgacatctggggccagggcacaatggtcacagtttctagcggaggcggaggatctggtggcggaggaagtggcggaggcggttctgagattgtgatgacacagagccccgccactctgagccttagtcctggcgaaagagccacactgagctgcagagccagccagagcatcaccagcgattacctgagctggtatcagcagaagcccggacaggctcccagactgctgatctatggcgcctctacaagagccaccggcattcccgcccgcttttctggctctggaagcggcaccgacttcaccctgaccatatctagcctgcagcctgaggacttcgtggtgtactattgccagcaggactacaacctgtacaccttcggccaggggaccaagctggaaatcaagaga

SEQ ID NO: 127 - protein, 244 aa, synthetic construct
  REGION 1..244: note = scFv (VH-VL)
QLQLQESGPGLVKPSETLSLTCTVSGGSINSTTSYWAWIRQPPGKGLEWIGTIFYSGKTYNNPSLKSRVTMSVDTSKNHFSLKVNSVTAADTAVYYCARFDYGFHDAFDIWGQGTMVTVSSGGGGSGGGGSGGGGSEIVMTQSPATLSLSPGERATLSCRASQSITSDYLSWYQQKPGQAPRLLIYGASTRATGIPARFSGSGSGTDFTLTISSLQPEDFVVYYCQQDYNLYTFGQGTKLEIKR

SEQ ID NO: 128 - DNA, 372 bp, synthetic construct
  misc_feature 1..372: note = VH
  source 1..372: mol_type = other DNA
caggttcagctgctgcagtctggagctgaggtgaagaagcctggggcctcagtgaaggtctcctgcaaggcttctggttacacctttaccagctatggtatcagctgggtgcgacaggcccctggacaagggcttgagtggatgggatggatcagcgcttacactggtaacacaaggtatgcacagaagctccagggcagagtcaccatgaccacagacacatccacgagcacagcctacatggagctgaggagcctgagatctgacgacacggccgtgtattactgtgcgagagaagaaggagctactacggactacgactactacggtatggacgtctggggccaagggactgcggtcaccgtctcctca

SEQ ID NO: 129 - DNA, 372 bp, synthetic construct
  misc_feature 1..372: note = VH O/SSE
  source 1..372: mol_type = other DNA
caggttcagctgcttcagtctggcgccgaagtgaagaaacctggcgcctctgtgaaggtgtcctgcaaggccagcggctacacctttaccagctacggcatcagctgggtccgacaggctcctggacaaggcttggaatggatgggctggatcagcgcctacaccggcaataccagatacgcccagaaactgcagggcagagtgaccatgaccaccgacaccagcacaagcaccgcctacatggaactgcggagcctgagatccgatgacaccgccgtgtactactgcgccagagaagaaggcgccaccaccgactacgactactacggcatggatgtgtggggccagggaacagccgtgacagtttcttct

SEQ ID NO: 130 - protein, 124 aa, synthetic construct
  REGION 1..124: note = VH
QVQLLQSGAEVKKPGASVKVSCKASGYTFTSYGISWVRQAPGQGLEWMGWISAYTGNTRYAQKLQGRVTMTTDTSTSTAYMELRSLRSDDTAVYYCAREEGATTDYDYYGMDVWGQGTAVTVSS

SEQ ID NO: 131 - DNA, 336 bp, synthetic construct
  misc_feature 1..336: note = VL O/SSE
  source 1..336: mol_type = other DNA
caactggttctgacacagagcccaagcgcctctgcatctctgggagcttccgtgaagctgacctgcacactgtctagcggccacagcagctatgccattgcctggcatcagcaacagcccgagaagggccctagatacctgatgaagctgaacagcgacggcagccactctaaaggcgacggcatccccgatagattcagcggcagttctagcggagccgagcgctacctgacaatcagctctctgcaatccgaggacgaggccgattactactgtcagacatggggcaccggcatcagagtgtttggcggcggaacaaagctgaccgtgctgggc

SEQ ID NO: 132 - DNA, 696 bp, synthetic construct
  misc_feature 1..696: note = scFv (VH-VL)
  source 1..696: mol_type = other DNA
caggttcagctgctgcagtctggagctgaggtgaagaagcctggggcctcagtgaaggtctcctgcaaggcttctggttacacctttaccagctatggtatcagctgggtgcgacaggcccctggacaagggcttgagtggatgggatggatcagcgcttacactggtaacacaaggtatgcacagaagctccagggcagagtcaccatgaccacagacacatccacgagcacagcctacatggagctgaggagcctgagatctgacgacacggccgtgtattactgtgcgagagaagaaggagctactacggactacgactactacggtatggacgtctggggccaagggactgcggtcaccgtctcctcaggtggtggtggtagcggcggcggcggctctggtggtggtggatcccagcttgtgctgactcaatcgccctctgcctctgcctccctgggagcctcggtcaagctcacctgcactctgagcagtgggcacagcagctacgccatcgcatggcatcagcagcagccagagaagggccctcggtacttgatgaagcttaacagtgatggcagccacagcaagggggacgggatccctgatcgcttctcaggctccagctctggggctgagcgctacctcaccatctccagcctccagtctgaggatgaggctgactattactgtcag

SEQ ID NO: 133 - DNA, 753 bp, synthetic construct
  misc_feature 1..753: note = scFv (VH-VL) O/SSE
  source 1..753: mol_type = other DNA
caggttcagctgcttcagtctggcgccgaagtgaagaaacctggcgcctctgtgaaggtgtcctgcaaggccagcggctacacctttaccagctacggcatcagctgggtccgacaggctcctggacaaggcttggaatggatgggctggatcagcgcctacaccggcaataccagatacgcccagaaactgcagggcagagtgaccatgaccaccgacaccagcacaagcaccgcctacatggaactgcggagcctgagatccgatgacaccgccgtgtactactgcgccagagaagaaggcgccaccaccgactacgactactacggcatggatgtgtggggccagggaacagccgtgacagtttcttctggtggcggaggatctggcggaggtggaagcggcggaggcggatctcaactggttctgacacagagcccaagcgcctctgcatctctgggagcttccgtgaagctgacctgcacactgtctagcggccacagcagctatgccattgcctggcatcagcaacagcccgagaagggccctagatacctgatgaagctgaacagcgacggcagccactctaaaggcgacggcatccccgatagattcagcggcagttctagcggagccgagcgctacctgacaatcagctctctgcaatccgaggacgaggccgattactactgtcagacatggggcaccggcatcagagtgtttggcggcggaacaaagctgaccgtgctgggc

SEQ ID NO: 134 - protein, 251 aa, synthetic construct
  REGION 1..251: note = scFv (VH-VL)
QVQLLQSGAEVKKPGASVKVSCKASGYTFTSYGISWVRQAPGQGLEWMGWISAYTGNTRYAQKLQGRVTMTTDTSTSTAYMELRSLRSDDTAVYYCAREEGATTDYDYYGMDVWGQGTAVTVSSGGGGSGGGGSGGGGSQLVLTQSPSASASLGASVKLTCTLSSGHSSYAIAWHQQQPEKGPRYLMKLNSDGSHSKGDGIPDRFSGSSSGAERYLTISSLQSEDEADYYCQTWGTGIRVFGGGTKLTVLG

SEQ ID NO: 135 - protein, 13 aa, synthetic construct
  REGION 1..13: note = IgG4 hinge spacer
ESKYGPPCPPCPM

SEQ ID NO: 136 - DNA, 39 bp, synthetic construct
  misc_feature 1..39: note = IgG4 hinge spacer O/SSE
  source 1..39: mol_type = other DNA
gagtctaaatacggaccgccttgtcctccttgtcccatg

SEQ ID NO: 137 - DNA, 357 bp, synthetic construct
  misc_feature 1..357: note = Hinge-CH3 spacer O/SSE
  source 1..357: mol_type = other DNA
gagtctaaatacggaccgccttgtcctccttgtcccggccagccaagagagccccaggtttacacactgcctccaagccaagaggaaatgaccaagaatcaggtgtccctgacatgcctggtcaagggcttctacccctccgatatcgccgtggaatgggagagcaatggccagcctgagaacaactacaagaccacacctcctgtgctggacagcgacggcagtttcttcctgtatagtagactcaccgtggataaatcaagatggcaagagggcaacgtgttcagctgcagcgtgatgcacgaggccctgcacaaccactacacccagaaaagcctgagcctgtctctgggcaag

SEQ ID NO: 138 - protein, 120 aa, synthetic construct
  REGION 1..120: note = Hinge-CH3 spacer
ESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKM

SEQ ID NO: 139 - DNA, 360 bp, synthetic construct
  misc_feature 1..360: note = Hinge-CH3 spacer O/SSE
  source 1..360: mol_type = other DNA
gagtctaaatacggaccgccttgtcctccttgtcccggccagccaagagagccccaggtttacacactgcctccaagccaagaggaaatgaccaagaatcaggtgtccctgacatgcctggtcaagggcttctacccctccgatatcgccgtggaatgggagagcaatggccagcctgagaacaactacaagaccacacctcctgtgctggacagcgacggcagtttcttcctgtatagtagactcaccgtggataaatcaagatggcaagagggcaacgtgttcagctgcagcgtgatgcacgaggccctgcacaaccactacacccagaaaagcctgagcctgtctctgggcaagatg

SEQ ID NO: 140 - DNA, 684 bp, synthetic construct
  misc_feature 1..684: note = IgG4/IgG2 hinge- IgG2/IgG4 CH2- IgG4 CH3 spacer O/SSE
  source 1..684: mol_type = other DNA
gagtctaaatacggaccgccttgtcctccttgtcccgctcctcctgttgccggaccttccgtgttcctgtttcctccaaagcctaaggacaccctgatgatcagcaggacccctgaagtgacctgcgtggtggtggatgtgtcccaagaggatcccgaggtgcagttcaactggtatgtggacggcgtggaagtgcacaacgccaagaccaagcctagagaggaacagttccagagcacctacagagtggtgtccgtgctgacagtgctgcaccaggattggctgaacggcaaagagtacaagtgcaaggtgtccaacaagggcctgcctagcagcatcgagaaaaccatctccaaggccaagggccagccaagagagccccaggtttacacactgcctccaagccaagaggaaatgaccaagaatcaggtgtccctgacatgcctggtcaagggcttctacccctccgatatcgccgtggaatgggagagcaatggccagcctgagaacaactacaagaccacacctcctgtgctggacagcgacggcagtttcttcctgtatagtagactcaccgtggataaatcaagatggcaagagggcaacgtgttcagctgcagcgtgatgcacgaggccctgcacaaccactacacccagaaaagcctgagcctgtctctgggcaag

SEQ ID NO: 141 - DNA, 72 bp, synthetic construct
  misc_feature 1..72: note = T2A O/SSE
  source 1..72: mol_type = other DNA
cttgaaggtggtggcgaaggcagaggcagcctgcttacatgcggagatgtggaagagaaccccggacctaga

SEQ ID NO: 142 - protein, 248 aa, synthetic construct
  REGION 1..248: note = R12 VH-VL scFv
QEQLVESGGRLVTPGGSLTLSCKASGFDFSAYYMSWVRQAPGKGLEWIATIYPSSGKTYYATWVNGRFTISSDNAQNTVDLQMNSLTAADRATYFCARDSYADDGALFNIWGPGTLVTISSGGGGSGGGGSGGGGSELVLTQSPSVSAALGSPAKITCTLSSAHKTDTIDWYQQLQGEAPRYLMQVQSDGSYTKRPGVPDRFSGSSSGADRYLIIPSVQADDEADYYCGADYIGGYVFGGGTQLTVTG

SEQ ID NO: 143 - DNA, 745 bp, synthetic construct
  misc_feature 1..745: note = R12 VH-VL scFv
  source 1..745: mol_type = other DNA
caagaacagctggtggaatctggcggcagactggttacacctggcggaagcctgacactgagctgtaaagccagcggcttcgacttcagcgcctactacatgagctgggtccgacaggcccctggcaaaggactggaatggatcgccacaatctaccccagctccggcaagacctactacgccacatgggtcaacggccggttcaccatcagcagcgacaacgcccagaacaccgtggacctgcagatgaactctctgacagccgccgaccgggccacctacttttgtgccagagatagctacgccgacgacggcgccctgttcaatatttggggacctggcacactcgtgaccatctctagcggaggcggaggaagtggtggcggaggatcaggcggtggtggatctgaactggtgctgacacagagcccctctgtgtctgctgctctgggaagccctgccaagatcacatgtaccctgagcagcgcccacaagaccgacaccatcgactggtatcagcagctgcagggcgaagcccctagatacctgatgcaggttcagagcgacggcagctacaccaaaagacctggcgtgcccgatagattcagcggcagttcttctggcgccgatcgctacctgatcatcccttctgtgcaagccgacgatgaggccgactattactgcggagccgattacatcggcggctacgttttcggtggcggcacacagttgacagtgacaggcg

SEQ ID NO: 144 - protein, 908 aa, Homo sapiens
  REGION 1..908: note = MISC_FEATURE - human ROR1 GenBank: AAA60275.1
QETELSVSAELVPTSSWNISSELNKDSYLTLDEPMNNITTSLGQTAELHCKVSGNPPPTIRWFKNDAPVVQEPRRLSFRSTIYGSRLRIRNLDTTDTGYFQCVATNGKEVVSSTGVLFVKFGPPPTASPGYSDEYEEDGFCQPYRGIACARFIGNRTVYMESLHMQGEIENQITAAFTMIGTSSHLSDKCSQFAIPSLCHYAFPYCDETSSVPKPRDLCRDECEILENVLCQTEYIFARSNPMILMRLKLPNCEDLPQPESPEAANCIRIGIPMADPINKNHKCYNSTGVDYRGTVSVTKSGRQCQPWNSQYPHTHTFTALRFPELNGGHSYCRNPGNQKEAPWCFTLDENFKSDLCDIPACDSKDSKEKNKMEILYILVPSVAIPLAIALLFFFICVCRNNQKSSSAPVQRQPKHVRGQNVEMSMLNAYKPKSKAKELPLSAVRFMEELGECAFGKIYKGHLYLPGMDHAQLVAIKTLKDYNNPQQWTEFQQEASLMAELHHPNIVCLLGAVTQEQPVCMLFEYINQGDLHEFLIMRSPHSDVGCSSDEDGTVKSSLDHGDFLHIAIQIAAGMEYLSSHFFVHKDLAARNILIGEQLHVKISDLGLSREIYSADYYRVQSKSLLPIRWMPPEAIMYGKFSSDSDIWSFGVVLWEIFSFGLQPYYGFSNQEVIEMVRKRQLLPCSEDCPPRMYSLMTECWNEIPSRRPRFKDIHVRLRSWEGLSSHTSSTTPSGGNATTQTTSLSASPVSNLSNPRYPNYMFPSQGITPQGQIAGFIGPPIPQNQRFIPINGYPIPPGYAAFPAAHYQPTGPPRVIQHCPPPKSRSPSSASGSTSTGHVTSLPSSGSNQEANIPLLPHMSIPNHPGGMGITVFGNKSQKPYKIDSKQASLLGDANIHGHTESMISAEL

SEQ ID NO: 145 - protein, 520 aa, Homo sapiens
  REGION 1..520: note = MISC_FEATURE - human ROR1 isoform 2
QETELSVSAELVPTSSWNISSELNKDSYLTLDEPMNNITTSLGQTAELHCKVSGNPPPTIRWFKNDAPVVQEPRRLSFRSTIYGSRLRIRNLDTTDTGYFQCVATNGKEVVSSTGVLFVKFGPPPTASPGYSDEYEEDGFCQPYRGIACARFIGNRTVYMESLHMQGEIENQITAAFTMIGTSSHLSDKCSQFAIPSLCHYAFPYCDETSSVPKPRDLCRDECEILENVLCQTEYIFARSNPMILMRLKLPNCEDLPQPESPEAANCIRIGIPMADPINKNHKCYNSTGVDYRGTVSVTKSGRQCQPWNSQYPHTHTFTALRFPELNGGHSYCRNPGNQKEAPWCFTLDENFKSDLCDIPACDSKDSKEKNKMEILYILVPSVAIPLAIALLFFFICVCRNNQKSSSAPVQRQPKHVRGQNVEMSMLNAYKPKSKAKELPLSAVRFMEELGECAFGKIYKGHLYLPGMDHAQLVAIKTLKDYNNPQQWTEFQQEASLMAELHHPNIVCLLGAVTQEQPVC

SEQ ID NO: 146 - protein, 364 aa, Homo sapiens
  REGION 1..364: note = MISC_FEATURE - human ROR1 isoform 3
QETELSVSAELVPTSSWNISSELNKDSYLTLDEPMNNITTSLGQTAELHCKVSGNPPPTIRWFKNDAPVVQEPRRLSFRSTIYGSRLRIRNLDTTDTGYFQCVATNGKEVVSSTGVLFVKFGPPPTASPGYSDEYEEDGFCQPYRGIACARFIGNRTVYMESLHMQGEIENQITAAFTMIGTSSHLSDKCSQFAIPSLCHYAFPYCDETSSVPKPRDLCRDECEILENVLCQTEYIFARSNPMILMRLKLPNCEDLPQPESPEAANCIRIGIPMADPINKNHKCYNSTGVDYRGTVSVTKSGRQCQPWNSQYPHTHTFTALRFPELNGGHSYCRNPGNQKEAPWCFTLDENFKSDLCDIPACGK

SEQ ID NO: 147 - DNA, 84 bp, synthetic construct
  misc_feature 1..84: note = CD28 transmembrane domain
  source 1..84: mol_type = other DNA
atgttttgggtgctggtcgtggtcggaggggtgctggcctgttacagcctgctggtgacagtcgctttcatcatcttctgggtg

SEQ ID NO: 148 - DNA, 84 bp, synthetic construct
  misc_feature 1..84: note = CD28 transmembrane domain (O/SSE)
  source 1..84: mol_type = other DNA
atgttctgggtgctcgtggtcgttggcggagtgctggcctgttacagcctgctggttaccgtggccttcatcatcttttgggtc

SEQ ID NO: 149 - protein, 28 aa, synthetic construct
  REGION 1..28: note = CD28 transmembrane domain
MFWVLVVVGGVLACYSLLVTVAFIIFWV

SEQ ID NO: 150 - DNA, 336 bp, synthetic construct
  misc_feature 1..336: note = CD3-zeta derived intracellular signaling domain (O/SSE)
  source 1..336: mol_type = other DNA
agagtgaagttcagcagatccgccgacgctccagcctatcagcagggccaaaaccagctgtacaacgagctgaacctggggagaagagaagagtacgacgtgctggataagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgacgcactgcacatgcaggccctgccacctaga

SEQ ID NO: 151 - DNA, 1074 bp, synthetic construct
  misc_feature 1..1074: note = truncated EGFR (tEGFR) sequence
  source 1..1074: mol_type = other DNA
atgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtga

SEQ ID NO: 152 - DNA, 1074 bp, synthetic construct
  misc_feature 1..1074: note = truncated EGFR (tEGFR) sequence (O/SSE)
  source 1..1074: mol_type = other DNA
atgctgctcctcgtgacaagcctgctcctgtgtgaactccctcatccagcttttctgctcattcctcggaaagtgtgcaacggcatcggcatcggagagttcaaggacagcctgagcatcaatgccaccaacatcaagcacttcaagaattgcaccagcatcagcggcgacctgcacattctgcctgtggcctttagaggcgacagcttcacccacacacctccactggatccccaagagctggatatcctgaaaaccgtgaaagagattaccggattcctcctgatccaagcctggccagagaacagaaccgatctgcacgccttcgagaacctcgagatcatcagaggccggaccaaacagcacggccagtttagcctggctgtggtgtctctgaacatcaccagtctgggcctgagaagcctgaaagaaatctccgacggcgacgtgatcatctccggaaacaagaacctgtgctacgccaacaccatcaactggaagaagctgttcggcacctccggccagaaaacaaagatcatctctaaccggggcgagaacagctgcaaggccaccggacaagtttgtcacgccctgtgtagccctgaaggctgttggggacccgaacctagagactgtgtgtcctgccggaatgtgtcccggggcagagaatgtgtggataagtgcaacctgctggaaggcgagccccgcgagtttgtggaaaacagcgagtgcatccagtgtcaccccgagtgtctgccccaggccatgaacattacatgcaccggcagaggccccgacaactgtattcagtgcgcccactacatcgacggccctcactgcgtgaaaacatgtccagctggcgtgatgggagagaacaacaccctcgtgtggaagtatgccgacgccggacatgtgtgccacctgtgtcaccctaattgcacctacggctgtaccggacctggcctggaaggatgccctacaaacggccctaagatccccagcattgccaccggaatggttggagccctgctgcttctgttggtggtggccctcggaatcggcctgttcatgtga

SEQ ID NO: 153 - protein, 357 aa, synthetic construct; identical to SEQ ID NO: 7
  REGION 1..357: note = truncated EGFR (tEGFR) sequence

SEQ ID NO: 154 - DNA, 126 bp, synthetic construct
  misc_feature 1..126: note = 4-1BB intracellular co-signaling sequence
  source 1..126: mol_type = other DNA
aagcgggggagaaagaaactgctgtatattttcaaacagccctttatgagacctgtgcagactacccaggaggaagacggatgcagctgtaggtttcccgaggaagaggaaggaggctgtgagctg

SEQ ID NO: 155 - DNA, 126 bp, synthetic construct
  misc_feature 1..126: note = 4-1BB intracellular co-signaling sequence (O/SSE)
  source 1..126: mol_type = other DNA
aagcggggcagaaagaagctgctctacatcttcaagcagcccttcatgcggcccgtgcagaccacacaagaggaagatggctgctcctgcagattccccgaggaagaagaaggcggctgcgagctg

SEQ ID NO: 156 - DNA, 1308 bp, synthetic construct
  misc_feature 1..1308: note = anti-ROR1 CAR
  source 1..1308: mol_type = other DNA
caggttcagctgcaagagtctggccctggcctggtcaagcctagcgaaacactgagcctgacctgtaccgtgtctggcggcagcatctccaactactactggtcctggatcagacagcctgccggcaaaggcctggaatggatcggcagaatctacaccagcggcagcaccaactacaaccccagcctgaagtccagagtgaccatgagcgtggacaccagcaagaaccagttctccctgaagctgagcagcctgacagccgccgataccgccatctactactgtgcccggtactacgatatcctgaccggcttcttcgactactggggccagggaacactggtcacagtttctagcggaggcggaggatctggtggcggaggaagtggcggaggcggttctgtgatttggatgacacagagccctagcctgctgagcgccagcacaggcgatagcgtgaccatcagctgcagaatgagccaggacatcagcagctacctggcttggtatcagcagaagcctggcaaggcccctgaactgctgatctatgccgcttccagtctgcagagcggcgtgccatctagattttccggcagcggctctggcaccgacttcaccctgacaatcagctccctgcagtccgaggacttcgccacctactattgccagcagtacgacagcttccctccaacctttggccagggcaccaaggtggaattcaagcgcgagtctaaatacggaccgccttgtcctccttgtcccatgttctgggtgctcgtggtcgttggcggagtgctggcctgttacagcctgctggttaccgtggccttcatcatcttttgggtcaagcggggcagaaagaagctgctctacatcttcaagcagcccttcatgcggcccgtgcagaccacacaagaggaagatggctgctcctgcagattccccgaggaagaagaaggcggctgcgagctgagagtgaagttcagcagatccgccgacgctccagcctatcagcagggccaaaaccagctgtacaacgagctgaacctggggagaagagaagagtacgacgtgctggataagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgacgcactgcacatgcaggccctgccacctaga

SEQ ID NO: 157 - DNA, 1314 bp, synthetic construct
  misc_feature 1..1314: note = anti-ROR1 CAR
  source 1..1314: mol_type = other DNA
cagctccagctgcaagaatctggacctggcctggtcaagcccagcgagacactgtctctgacctgtacagtgtccggcggcagcatcaatagcaccacaagctactgggcctggatcagacagcctcctggcaaaggcctggaatggatcggcaccatcttctacagcggcaagacctacaacaaccccagcctgaagtccagagtgaccatgagcgtggacaccagcaagaaccacttcagcctgaaagtgaacagcgtgacagccgccgataccgccgtgtactactgcgccagattcgactacggcttccacgacgccttcgacatctggggccagggcacaatggtcacagtttctagcggaggcggaggatctggtggcggaggaagtggcggaggcggttctgagattgtgatgacacagagccccgccactctgagccttagtcctggcgaaagagccacactgagctgcagagccagccagagcatcaccagcgattacctgagctggtatcagcagaagcccggacaggctcccagactgctgatctatggcgcctctacaagagccaccggcattcccgcccgcttttctggctctggaagcggcaccgacttcaccctgaccatatctagcctgcagcctgaggacttcgtggtgtactattgccagcaggactacaacctgtacaccttcggccaggggaccaagctggaaatcaagagagagtctaaatacggaccgccttgtcctccttgtcccatgttctgggtgctcgtggtcgttggcggagtgctggcctgttacagcctgctggttaccgtggccttcatcatcttttgggtcaagcggggcagaaagaagctgctctacatcttcaagcagcccttcatgcggcccgtgcagaccacacaagaggaagatggctgctcctgcagattccccgaggaagaagaaggcggctgcgagctgagagtgaagttcagcagatccgccgacgctccagcctatcagcagggccaaaaccagctgtacaacgagctgaacctggggagaagagaagagtacgacgtgctggataagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgacgcactgcacatgcaggccctgccacctaga

SEQ ID NO: 158 - DNA, 1629 bp, synthetic construct
  misc_feature 1..1629: note = anti-ROR1 CAR
  source 1..1629: mol_type = other DNA
caggttcagctgcaagagtctggccctggcctggtcaagcctagcgaaacactgagcctgacctgtaccgtgtctggcggcagcatctccaactactactggtcctggatcagacagcctgccggcaaaggcctggaatggatcggcagaatctacaccagcggcagcaccaactacaaccccagcctgaagtccagagtgaccatgagcgtggacaccagcaagaaccagttctccctgaagctgagcagcctgacagccgccgataccgccatctactactgtgcccggtactacgatatcctgaccggcttcttcgactactggggccagggaacactggtcacagtttctagcggaggcggaggatctggtggcggaggaagtggcggaggcggttctgtgatttggatgacacagagccctagcctgctgagcgccagcacaggcgatagcgtgaccatcagctgcagaatgagccaggacatcagcagctacctggcttggtatcagcagaagcctggcaaggcccctgaactgctgatctatgccgcttccagtctgcagagcggcgtgccatctagattttccggcagcggctctggcaccgacttcaccctgacaatcagctccctgcagtccgaggacttcgccacctactattgccagcagtacgacagcttccctccaacctttggccagggcaccaaggtggaattcaagcgcgagtctaaatacggaccgccttgtcctccttgtcccggccagccaagagagccccaggtttacacactgcctccaagccaagaggaaatgaccaagaatcaggtgtccctgacatgcctggtcaagggcttctacccctccgatatcgccgtggaatgggagagcaatggccagcctgagaacaactacaagaccacacctcctgtgctggacagcgacggcagtttcttcctgtatagtagactcaccgtggataaatcaagatggcaagagggcaacgtgttcagctgcagcgtgatgcacgaggccctgcacaaccactacacccagaaaagcctgagcctgtctctgggcaagatgttctgggtgctcgtggtcgttggcggagtgctggcctgttacagcctgctggttaccgtggccttcatcatcttttgggtcaagcggggcagaaagaagctgctctacatcttcaagcagcccttcatgcggcccgtgcagaccacacaagaggaagatggctgctcctgcagattccccgaggaagaagaaggcggctgcgagctgagagtgaagttcagcagatccgccgacgctccagcctatcagcagggccaaaaccagctgtacaacgagctgaacctggggagaagagaagagtacgacgtgctggataagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgacgcactgcacatgcaggccctgccacctaga

SEQ ID NO: 159 - DNA, 1656 bp, synthetic construct
  misc_feature 1..1656: note = anti-ROR1 CAR
  source 1..1656: mol_type = other DNA
caggtgcagctggttcaatctggcgccgaagtgaagaaaccaggcgcctctgtgaaggtgtcctgcaaggccagcggctacacctttaccagctacggcatcagctgggtccgacaggctcctggacaaggcttggaatggatgggctggatcagcgcctacaacggcaacaccaaatacgcccagaaactgcagggcagagtgaccatgaccaccgacaccagcacaagcaccgcctacatggaactgcggagcctgagatccgatgacaccgccgtgtactactgcgccagagatgaggacatcctgaccggctacaactactacggcatggacgtgtggggccagggcacaacagtgacagtttcttctggcggcggaggatctggcggaggtggaagcggaggcggtggatctcaactggtgctgacacagtctcctagcgcctctgcttctctgggagccagcgtgaagctgacctgtacactgtctagcggccacagcagctacgccattgcttggcatcagcagcagcccgagaagggccctagatacctgatgaagctgaacagcgacggcagccactctaaaggcgacggcatccccgatagattcagcggcagttctagcggagccgagcgctacctgacaatcagctctctgcaatccgaggacgaggccgactactactgtcagacatggggcaccggcatcagagtgtttggcggaggcaccaagctgacagtgcttggagagtctaaatacggaccgccttgtcctccttgtcccggccagccaagagagccccaggtttacacactgcctccaagccaagaggaaatgaccaagaatcaggtgtccctgacatgcctggtcaagggcttctacccctccgatatcgccgtggaatgggagagcaatggccagcctgagaacaactacaagaccacacctcctgtgctggacagcgacggcagtttcttcctgtatagtagactcaccgtggataaatcaagatggcaagagggcaacgtgttcagctgcagcgtgatgcacgaggccctgcacaaccactacacccagaaaagcctgagcctgtctctgggcaagatgttctgggtgctcgtggtcgttggcggagtgctggcctgttacagcctgctggttaccgtggccttcatcatcttttgggtcaagcggggcagaaagaagctgctctacatcttcaagcagcccttcatgcggcccgtgcagaccacacaagaggaagatggctgctcctgcagattccccgaggaagaagaaggcggctgcgagctgagagtgaagttcagcagatccgccgacgctccagcctatcagcagggccaaaaccagctgtacaacgagctgaacctggggagaagagaagagtacgacgtgctggataagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgacgcactgcacatgcaggccctgccacctaga

SEQ ID NO: 160 - DNA, 1635 bp, synthetic construct
  misc_feature 1..1635: note = anti-ROR1 CAR
  source 1..1635: mol_type = other DNA
cagctccagctgcaagaatctggacctggcctggtcaagcccagcgagacactgtctctgacctgtacagtgtccggcggcagcatcaatagcaccacaagctactgggcctggatcagacagcctcctggcaaaggcctggaatggatcggcaccatcttctacagcggcaagacctacaacaaccccagcctgaagtccagagtgaccatgagcgtggacaccagcaagaaccacttcagcctgaaagtgaacagcgtgacagccgccgataccgccgtgtactactgcgccagattcgactacggcttccacgacgccttcgacatctggggccagggcacaatggtcacagtttctagcggaggcggaggatctggtggcggaggaagtggcggaggcggttctgagattgtgatgacacagagccccgccactctgagccttagtcctggcgaaagagccacactgagctgcagagccagccagagcatcaccagcgattacctgagctggtatcagcagaagcccggacaggctcccagactgctgatctatggcgcctctacaagagccaccggcattcccgcccgcttttctggctctggaagcggcaccgacttcaccctgaccatatctagcctgcagcctgaggacttcgtggtgtactattgccagcaggactacaacctgtacaccttcggccaggggaccaagctggaaatcaagagagagtctaaatacggaccgccttgtcctccttgtcccggccagccaagagagccccaggtttacacactgcctccaagccaagaggaaatgaccaagaatcaggtgtccctgacatgcctggtcaagggcttctacccctccgatatcgccgtggaatgggagagcaatggccagcctgagaacaactacaagaccacacctcctgtgctggacagcgacggcagtttcttcctgtatagtagactcaccgtggataaatcaagatggcaagagggcaacgtgttcagctgcagcgtgatgcacgaggccctgcacaaccactacacccagaaaagcctgagcctgtctctgggcaagatgttctgggtgctcgtggtcgttggcggagtgctggcctgttacagcctgctggttaccgtggccttcatcatcttttgggtcaagcggggcagaaagaagctgctctacatcttcaagcagcccttcatgcggcccgtgcagaccacacaagaggaagatggctgctcctgcagattccccgaggaagaagaaggcggctgcgagctgagagtgaagttcagcagatccgccgacgctccagcctatcagcagggccaaaaccagctgtacaacgagctgaacctggggagaagagaagagtacgacgtgctggataagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgacgcactgcacatgcaggccctgccacctaga

SEQ ID NO: 161 - DNA, 1656 bp, Homo sapiens
  source 1..1656: mol_type = unassigned DNA
caggttcagctgcttcagtctggcgccgaagtgaagaaacctggcgcctctgtgaaggtgtcctgcaaggccagcggctacacctttaccagctacggcatcagctgggtccgacaggctcctggacaaggcttggaatggatgggctggatcagcgcctacaccggcaataccagatacgcccagaaactgcagggcagagtgaccatgaccaccgacaccagcacaagcaccgcctacatggaactgcggagcctgagatccgatgacaccgccgtgtactactgcgccagagaagaaggcgccaccaccgactacgactactacggcatggatgtgtggggccagggaacagccgtgacagtttcttctggtggcggaggatctggcggaggtggaagcggcggaggcggatctcaactggttctgacacagagcccaagcgcctctgcatctctgggagcttccgtgaagctgacctgcacactgtctagcggccacagcagctatgccattgcctggcatcagcaacagcccgagaagggccctagatacctgatgaagctgaacagcgacggcagccactctaaaggcgacggcatccccgatagattcagcggcagttctagcggagccgagcgctacctgacaatcagctctctgcaatccgaggacgaggccgattactactgtcagacatggggcaccggcatcagagtgtttggcggcggaacaaagctgaccgtgctgggcgagtctaaatacggaccgccttgtcctccttgtcccggccagccaagagagccccaggtttacacactgcctccaagccaagaggaaatgaccaagaatcaggtgtccctgacatgcctggtcaagggcttctacccctccgatatcgccgtggaatgggagagcaatggccagcctgagaacaactacaagaccacacctcctgtgctggacagcgacggcagtttcttcctgtatagtagactcaccgtggataaatcaagatggcaagagggcaacgtgttcagctgcagcgtgatgcacgaggccctgcacaaccactacacccagaaaagcctgagcctgtctctgggcaagatgttctgggtgctcgtggtcgttggcggagtgctggcctgttacagcctgctggttaccgtggccttcatcatcttttgggtcaagcggggcagaaagaagctgctctacatcttcaagcagcccttcatgcggcccgtgcagaccacacaagaggaagatggctgctcctgcagattccccgaggaagaagaaggcggctgcgagctgagagtgaagttcagcagatccgccgacgctccagcctatcagcagggccaaaaccagctgtacaacgagctgaacctggggagaagagaagagtacgacgtgctggataagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgacgcactgcacatgcaggccctgccacctaga

SEQ ID NO: 162 - protein, 21 aa, synthetic construct
  REGION 1..21: note = linker
SRGGGGSGGGGSGGGGSLEMA

SEQ ID NO: 163 - protein, 22 aa, synthetic construct
  REGION 1..22: note = linker
GSRGGGGSGGGGSGGGGSLEMA

SEQ ID NO: 164 - DNA, 544 bp, synthetic construct
  misc_feature 1..544: note = EF1alpha promoter with HTLV1 ehancer
  source 1..544: mol_type = other DNA
ggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctac

SEQ ID NO: 165 - DNA, 588 bp, synthetic construct
  misc_feature 1..588: note = Woodchuck Hepatitis Virus (WHP) Posttranscriptional Regulatory Element (WPRE)
  source 1..588: mol_type = other DNA
atcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc

SEQ ID NO: 166 - protein, 21 aa, synthetic construct
  REGION 1..21: note = T2A peptide
GSGEGRGSLLTCGDVEENPGP

SEQ ID NO: 167 - protein, 23 aa, synthetic construct
  REGION 1..23: note = E2A peptide
GSGQCTNYALLKLAGDVESNPGP

SEQ ID NO: 168 - protein, 25 aa, synthetic construct
  REGION 1..25: note = F2A peptide
GSGVKQTLNFDLLKLAGDVESNPGP

SEQ ID NO: 169 - DNA, 72 bp, synthetic construct
  misc_feature 1..72: note = T2A peptide
  source 1..72: mol_type = other DNA
ctcgagggcggcggagagggcagaggaagtcttctaacatgcggtgacgtggaggagaatcccggccctagg

SEQ ID NO: 170 - DNA, 3358 bp, Homo sapiens
  misc_feature 1..3358: note = human ROR1 GenBank: M97675.1
  source 1..3358: mol_type = unassigned DNA
gagctggagcagccgccaccgccgccgccgagggagccccgggacggcagcccctgggcgcagggtgcgctgttctcggagtccgacccagggcgactcacgcccactggtgcgacccggacagcctgggactgacccgccggcccaggcgaggctgcagccagagggctgggaagggatcgcgctcgcggcatccagaggcggccaggcggaggcgagggagcaggttagagggacaaagagctttgcagacgtccccggcgtcctgcgagcgccagcggccgggacgaggcggccgggagcccgggaagagcccgtggatgttctgcgcgcggcctgggagccgccgccgccgccgcctcagcgagaggaggaatgcaccggccgcgccgccgcgggacgcgcccgccgctcctggcgctgctggccgcgctgctgctggccgcacgcggggctgctgcccaagaaacagagctgtcagtcagtgctgaattagtgcctacctcatcatggaacatctcaagtgaactcaacaaagattcttacctgacccttgatgaaccaatgaataacatcaccacgtctctgggccagacagcagaactgcactgcaaagtctctgggaatccacctcccaccatccgctggttcaaaaatgatgctcctgtggtccaggagccccggaggctctcctttcggtccaccatctatggctctcggctgcggattagaaacctcgacaccacagacacaggctacttccagtgcgtggcaacaaacggcaaggaggtggtttcttccactggagtcttgtttgtcaagtttggcccccctcccactgcaagtccaggatactcagatgagtatgaagaagatggattctgtcagccatacagagggattgcatgtgcaagatttattggcaaccgcaccgtctatatggagtctttgcacatgcaaggggaaatagaaaatcagatcacagctgccttcactatgattggcacttccagtcacttatctgataagtgttctcagttcgccattccttccctgtgccactatgccttcccgtactgcgatgaaacttcatccgtcccaaagccccgtgacttgtgtcgcgatgaatgtgaaatcctggagaatgtcctgtgtcaaacagagtacatttttgcaagatcaaatcccatgattctgatgaggctgaaactgccaaactgtgaagatctcccccagccagagagcccagaagctgcgaactgtatccggattggaattcccatggcagatcctataaataaaaatcacaagtgttataacagcacaggtgtggactaccgggggaccgtcagtgtgaccaaatcagggcgccagtgccagccatggaattcccagtatccccacacacacactttcaccgcccttcgtttcccagagctgaatggaggccattcctactgccgcaacccagggaatcaaaaggaagctccctggtgcttcaccttggatgaaaactttaagtctgatctgtgtgacatcccagcttgcgattcaaaggattccaaggagaagaataaaatggaaatcctgtacatactagtgccaagtgtggccattcccctggccattgctttactcttcttcttcatttgcgtctgtcggaataaccagaagtcatcgtcggcaccagtccagaggcaaccaaaacacgtcagaggtcaaaatgtggagatgtcaatgctgaatgcatataaacccaagagcaaggctaaagagctacctctttctgctgtacgctttatggaagaattgggtgagtgtgcctttggaaaaatctataaaggccatctctatctcccaggcatggaccatgctcagctggttgctatcaagaccttgaaagactataacaacccccagcaatggatggaatttcaacaagaagcctccctaatggcagaactgcaccaccccaatattgtctgccttctaggtgccgtcactcaggaacaacctgtgtgcatgctttttgagtatattaatcagggggatctccatgagttcctcatcatgagatccccacactctgatgttggctgcagcagtgatgaagatgggactgtgaaatccagcctggaccacggagattttctgcacattgcaattcagattgcagctggcatggaatacctgtctagtcacttctttgtccacaaggaccttgcagctcgcaatattttaatcggagagcaacttcatgtaaagatttcagacttggggctttccagagaaatttactccgctgattactacagggtccagagtaagtccttgctgcccattcgctggatgccccctgaagccatcatgtatggcaaattctcttctgattcagatatctggtcctttggggttgtcttgtgggagattttcagttttggactccagccatattatggattcagtaaccaggaagtgattgagatggtgagaaaacggcagctcttaccatgctctgaagactgcccacccagaatgtacagcctcatgacagagtgctggaatgagattccttctaggagaccaagatttaaagatattcacgtccggcttcggtcctgggagggactctcaagtcacacaagctctactactccttcagggggaaatgccaccacacagacaacctccctcagtgccagcccagtgagtaatctcagtaaccccagatatcctaattacatgttcccgagccagggtattacaccacagggccagattgctggtttcattggcccgccaatacctcagaaccagcgattcattcccatcaatggatacccaatacctcctggatatgcagcgtttccagctgcccactaccagccaacaggtcctcccagagtgattcagcactgcccacctcccaagagtcggtccccaagcagtgccagtgggtcgactagcactggccatgtgactagcttgccctcatcaggatccaatcaggaagcaaatattcctttactaccacacatgtcaattccaaatcatcctggtggaatgggtatcaccgtttttggcaacaaatctcaaaaaccctacaaaattgactcaaagcaagcatctttactaggagacgccaatattcatggacacaccgaatctatgatttctgcagaactgtaaaatgcacaacttttgtaaatgtggtatacaggacaaactagacggccgtagaaaagatttatattcaaatgtttttattaaagtaaggttctcatttagcagacatcgcaacaagtaccttctgtgaagtttcactgtgtcttaccaagcaggacagacactcggccag

SEQ ID NO: 171 - protein, 908 aa, Homo sapiens
  REGION 1..908: note = MISC_FEATURE - Mouse ROR1; GenBank No. NP_038873
QETELSVSAELVPTSSWNTSSEIDKGSYLTLDEPMNNITTSLGQTAELHCKVSGNPPPSIRWFKNDAPVVQEPRRISFRATNYGSRLRIRNLDTTDTGYFQCVATNGKKVVSTTGVLFVKFGPPPTASPGSSDEYEEDGFCQPYRGIACARFIGNRTVYMESLHMQGEIENQITAAFTMIGTSSHLSDKCSQFAIPSLCHYAFPYCDETSSVPKPRDLCRDECEVLENVLCQTEYIFARSNPMILMRLKLPNCEDLPQPESPEAANCIRIGIPMADPINKNHKCYNSTGVDYRGTVSVTKSGRQCQPWNSQYPHTHSFTALRFPELNGGHSYCRNPGNQKEAPWCFTLDENFKSDLCDIPACDSKDSKEKNKMEILYILVPSVAIPLAIAFLFFFICVCRNNQKSSSPPVQRQPKPVRGQNVEMSMLNAYKPKSKAKELPLSAVRFMEELGECTFGKIYKGHLYLPGMDHAQLVAIKTLKDYNNPQQWTEFQQEASLMAELHHPNIVCLLGAVTQEQPVCMLFEYMNQGDLHEFLIMRSPHSDVGCSSDEDGTVKSSLDHGDFLHIAIQIAAGMEYLSSHFFVHKDLAARNILIGEQLHVKISDLGLSREIYSADYYRVQSKSSLPIRWMPPEAIMYGKFSSDSDIWSFGVVLWEIFSFGLQPYYGFSNQEVIEMVRKRQLLPCSEDCPPRMYSLMTECWNEIPSRRPRFKDIHVRLRSWEGLSSHTSSTTPSGGNATTQTTSLSASPVSNLSNPRFPNYMFPSQGITPQGQIAGFIGPAIPQNQRFIPINGYPIPPGYAAFPAAHYQPAGPPRVIQHCPPPKSRSPSSASGSTSTGHVASLPSSGSNQEANVPLLPHMSIPNHPGGMGITVFGNKSQKPYKIDSKQSSLLGDSHIHGHTESMISAEV

SEQ ID NO: 172 - protein, 118 aa, synthetic construct
  REGION 1..118: note = 2A2 VH
QVQLQQSGAELVRPGASVTLSCKASGYTFSDYEMHWVIQTPVHGLEWIGAIDPETGGTAYNQKFKGKAILTADKSSSTAYMELRSLTSEDSAVYYCTGYYDYDSFTYWGQGTLVTVSA

SEQ ID NO: 173 - protein, 107 aa, synthetic construct
  REGION 1..107: note = 2A2 VL
DIVMTQSQKIMSTTVGDRVSITCKASQNVDAAVAWYQQKPGQSPKLLIYSASNRYTGVPDRFTGSGSGTDFTLTISNMQSEDLADYFCQQYDIYPYTFGGGTKLEIK

SEQ ID NO: 174 - protein, 116 aa, synthetic construct
  REGION 1..116: note = 99961 humanized VH
QVQLQESGPGLVKPSQTLSLTCTVSGYAFTAYNIHWVRQAPGQGLEWMGSFDPYDGGSSYNQKFKDRLTISKDTSKNQVVLTMTNMDPVDTATYYCARGWYYFDYWGHGTLVTVSS

SEQ ID NO: 175 - protein, 116 aa, synthetic construct; identical to SEQ ID NO: 174
  REGION 1..116: note = 99961 humanized VH

SEQ ID NO: 176 - protein, 116 aa, synthetic construct
  REGION 1..116: note = 99961 humanized VH
QVQLQESGPGLVKPSQTLSLTCTVSGYAFTAYNIHWIRQPPGKGLEWIGSFDPYDGGSSYNQKFKDRLTISKDTSKNQVVLTMTNMDPVDTATYYCARGWYYFDYWGHGTLVTVSS

SEQ ID NO: 177 - protein, 116 aa, synthetic construct; identical to SEQ ID NO: 176
  REGION 1..116: note = 99961 humanized VH

SEQ ID NO: 178 - protein, 107 aa, synthetic construct
  REGION 1..107: note = 99961 humanized VL
DIVMTQTPLSLPVTPGEPASISCRASKSISKYLAWYQQKPGQAPRLLIYSGSTLQSGIPPRFSGSGYGTDFTLTINNIESEDAAYYFCQQHDESPYTFGEGTKVEIK

SEQ ID NO: 179 - protein, 107 aa, synthetic construct
  REGION 1..107: note = 99961 humanized VL
DVVMTQSPLSLPVTLGQPASISCRASKSISKYLAWYQQKPGKAPKLLIYSGSTLQSGIPPRFSGSGYGTDFTLTINNIESEDAAYYFCQQHDESPYTFGEGTKVEIK

SEQ ID NO: 180 - protein, 107 aa, synthetic construct; identical to SEQ ID NO: 178
  REGION 1..107: note = 99961 humanized VL

SEQ ID NO: 181 - protein, 107 aa, synthetic construct; identical to SEQ ID NO: 179
  REGION 1..107: note = 99961 humanized VL

SEQ ID NO: 182 - DNA, 336 bp, synthetic construct
  misc_feature 1..336: note = CD3-zeta derived intracellular signaling domain
  source 1..336: mol_type = other DNA
agagtcaagttttccaggtccgccgacgctccagcctaccagcaggggcagaaccagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaagg

SEQ ID NO: 183 - DNA, 123 bp, synthetic construct
  misc_feature 1..123: note = CD28 endodomain
  source 1..123: mol_type = other DNA
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 184 - protein, 436 aa, synthetic construct
  REGION 1..436: note = anti-ROR1 CAR
QVQLQESGPGLVKPSETLSLTCTVSGGSISNYYWSWIRQPAGKGLEWIGRIYTSGSTNYNPSLKSRVTMSVDTSKNQFSLKLSSLTAADTAIYYCARYYDILTGFFDYWGQGTLVTVSSGGGGSGGGGSGGGGSVIWMTQSPSLLSASTGDSVTISCRMSQDISSYLAWYQQKPGKAPELLIYAASSLQSGVPSRFSGSGSGTDFTLTISSLQSEDFATYYCQQYDSFPPTFGQGTKVEFKRESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 185 - protein, 438 aa, synthetic construct
  REGION 1..438: note = anti-ROR1 CAR
QLQLQESGPGLVKPSETLSLTCTVSGGSINSTTSYWAWIRQPPGKGLEWIGTIFYSGKTYNNPSLKSRVTMSVDTSKNHFSLKVNSVTAADTAVYYCARFDYGFHDAFDIWGQGTMVTVSSGGGGSGGGGSGGGGSEIVMTQSPATLSLSPGERATLSCRASQSITSDYLSWYQQKPGQAPRLLIYGASTRATGIPARFSGSGSGTDFTLTISSLQPEDFVVYYCQQDYNLYTFGQGTKLEIKRESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 186 - protein, 543 aa, synthetic construct
  REGION 1..543: note = anti-ROR1 CAR
QVQLQESGPGLVKPSETLSLTCTVSGGSISNYYWSWIRQPAGKGLEWIGRIYTSGSTNYNPSLKSRVTMSVDTSKNQFSLKLSSLTAADTAIYYCARYYDILTGFFDYWGQGTLVTVSSGGGGSGGGGSGGGGSVIWMTQSPSLLSASTGDSVTISCRMSQDISSYLAWYQQKPGKAPELLIYAASSLQSGVPSRFSGSGSGTDFTLTISSLQSEDFATYYCQQYDSFPPTFGQGTKVEFKRESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 187 - protein, 552 aa, synthetic construct
  REGION 1..552: note = anti-ROR1 CAR
QVQLVQSGAEVKKPGASVKVSCKASGYTFTSYGISWVRQAPGQGLEWMGWISAYNGNTKYAQKLQGRVTMTTDTSTSTAYMELRSLRSDDTAVYYCARDEDILTGYNYYGMDVWGQGTTVTVSSGGGGSGGGGSGGGGSQLVLTQSPSASASLGASVKLTCTLSSGHSSYAIAWHQQQPEKGPRYLMKLNSDGSHSKGDGIPDRFSGSSSGAERYLTISSLQSEDEADYYCQTWGTGIRVFGGGTKLTVLGESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 188 - protein, 545 aa, synthetic construct
  REGION 1..545: note = anti-ROR1 CAR
QLQLQESGPGLVKPSETLSLTCTVSGGSINSTTSYWAWIRQPPGKGLEWIGTIFYSGKTYNNPSLKSRVTMSVDTSKNHFSLKVNSVTAADTAVYYCARFDYGFHDAFDIWGQGTMVTVSSGGGGSGGGGSGGGGSEIVMTQSPATLSLSPGERATLSCRASQSITSDYLSWYQQKPGQAPRLLIYGASTRATGIPARFSGSGSGTDFTLTISSLQPEDFVVYYCQQDYNLYTFGQGTKLEIKRESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 189 - protein, 552 aa, synthetic construct
  REGION 1..552: note = anti-ROR1 CAR
QVQLLQSGAEVKKPGASVKVSCKASGYTFTSYGISWVRQAPGQGLEWMGWISAYTGNTRYAQKLQGRVTMTTDTSTSTAYMELRSLRSDDTAVYYCAREEGATTDYDYYGMDVWGQGTAVTVSSGGGGSGGGGSGGGGSQLVLTQSPSASASLGASVKLTCTLSSGHSSYAIAWHQQQPEKGPRYLMKLNSDGSHSKGDGIPDRFSGSSSGAERYLTISSLQSEDEADYYCQTWGTGIRVFGGGTKLTVLGESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 190 - DNA, 48 bp, synthetic construct
  misc_feature 1..48: note = CD33 signal sequence (O/SSE)
  source 1..48: mol_type = other DNA
atgcctctgctgctgcttctgcctcttctttgggctggtgctctggct

SEQ ID NO: 191 - DNA, 48 bp, synthetic construct
  misc_feature 1..48: note = CD33 signal sequence GenBank: M23197.1
  source 1..48: mol_type = other DNA
atgccgctgctgctactgctgcccctgctgtgggcaggggccctggct

SEQ ID NO: 192 - DNA, 39 bp, synthetic construct
  misc_feature 1..39: note = Spacer (IgG4 hinge)
  source 1..39: mol_type = other DNA
gaatctaagtacggaccgccctgccccccttgccctatg

SEQ ID NO: 193 - DNA, 357 bp, synthetic construct; identical to SEQ ID NO: 4
  misc_feature 1..357: note = Hinge-CH3 spacer
  source 1..357: mol_type = other DNA

SEQ ID NO: 194 - protein, 229 aa, synthetic construct
  REGION 1..229: note = IgG4/IgG2 hinge- IgG2/IgG4 CH2- IgG4 CH3 spacer
ESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKM

SEQ ID NO: 195 - DNA, 687 bp, synthetic construct
  misc_feature 1..687: note = IgG4/IgG2 hinge- IgG2/IgG4 CH2- IgG4 CH3 spacer
  source 1..687: mol_type = other DNA
gaatctaagtacggaccgccctgccctccctgccctgctcctcctgtggctggaccaagcgtgttcctgtttccacctaagcctaaagataccctgatgatttcccgcacacctgaagtgacttgcgtggtcgtggacgtgagccaggaggatccagaagtgcagttcaactggtacgtggacggcgtggaagtccacaatgctaagactaaaccccgagaggaacagtttcagtcaacttaccgggtcgtgagcgtgctgaccgtcctgcatcaggattggctgaacgggaaggagtataagtgcaaagtgtctaataagggactgcctagctccatcgagaaaacaattagtaaggcaaaagggcagcctcgagaaccacaggtgtataccctgccccctagccaggaggaaatgaccaagaaccaggtgtccctgacatgtctggtcaaaggcttctatccaagtgacatcgccgtggagtgggaatcaaatgggcagcccgagaacaattacaagaccacaccacccgtgctggactctgatggaagtttctttctgtattccaggctgaccgtggataaatctcgctggcaggagggcaacgtgttctcttgcagtgtcatgcacgaagccctgcacaatcattatacacagaagtcactgagcctgtccctgggcaaaatg

SEQ ID NO: 196 - DNA, 687 bp, synthetic construct
  misc_feature 1..687: note = IgG4/IgG2 hinge- IgG2/IgG4 CH2- IgG4 CH3 spacer O/SSE
  source 1..687: mol_type = other DNA
gagtctaaatacggaccgccttgtcctccttgtcccgctcctcctgttgccggaccttccgtgttcctgtttcctccaaagcctaaggacaccctgatgatcagcaggacccctgaagtgacctgcgtggtggtggatgtgtcccaagaggatcccgaggtgcagttcaactggtatgtggacggcgtggaagtgcacaacgccaagaccaagcctagagaggaacagttccagagcacctacagagtggtgtccgtgctgacagtgctgcaccaggattggctgaacggcaaagagtacaagtgcaaggtgtccaacaagggcctgcctagcagcatcgagaaaaccatctccaaggccaagggccagccaagagagccccaggtttacacactgcctccaagccaagaggaaatgaccaagaatcaggtgtccctgacatgcctggtcaagggcttctacccctccgatatcgccgtggaatgggagagcaatggccagcctgagaacaactacaagaccacacctcctgtgctggacagcgacggcagtttcttcctgtatagtagactcaccgtggataaatcaagatggcaagagggcaacgtgttcagctgcagcgtgatgcacgaggccctgcacaaccactacacccagaaaagcctgagcctgtctctgggcaagatg

SEQ ID NO: 197 - DNA, 81 bp, synthetic construct
  misc_feature 1..81: note = CD28 transmembrane domain
  source 1..81: mol_type = other DNA
ttttgggtgctggtcgtggtcggaggggtgctggcctgttacagcctgctggtgacagtcgctttcatcatcttctgggtg

SEQ ID NO: 198 - DNA, 81 bp, synthetic construct
  misc_feature 1..81: note = CD28 transmembrane domain (O/SSE)
  source 1..81: mol_type = other DNA
ttctgggtgctcgtggtcgttggcggagtgctggcctgttacagcctgctggttaccgtggccttcatcatcttttgggtc

SEQ ID NO: 199 - protein, 15 aa, synthetic construct
  REGION 1..15: note = ROR1 epitope 1
FRSTIYGSRLRIRNL

SEQ ID NO: 200 - protein, 13 aa, synthetic construct
  REGION 1..13: note = ROR1 epitope 2
LSVSAELVPTSSW

SEQ ID NO: 201 - protein, 14 aa, synthetic construct
  REGION 1..14: note = ROR1 epitope 3
HCKVSGNPPPTIRW

SEQ ID NO: 202 - protein, 26 aa, synthetic construct
  REGION 1..26: note = ROR1 epitope 4
WFKNDAPVVQEPRRLSFRSTIYGSRL

SEQ ID NO: 203 - protein, 9 aa, synthetic construct
  REGION 1..9: note = ROR1 epitope 5
VSSTGVLFV

SEQ ID NO: 204 - protein, 13 aa, synthetic construct
  REGION 1..13: note = ROR1 epitope 6
LFVKFGPPPTASP

SEQ ID NO: 205 - protein, 11 aa, synthetic construct
  REGION 1..11: note = ROR1 epitope 7
DEYEEDGFCQP

SEQ ID NO: 206 - protein, 14 aa, synthetic construct
  REGION 1..14: note = ROR1 epitope 8
QPYRGIACARFIGN

SEQ ID NO: 207 - protein, 14 aa, synthetic construct
  REGION 1..14: note = ROR1 epitope 9
SQFAIPSLCHYAFP

SEQ ID NO: 208 - protein, 12 aa, synthetic construct
  REGION 1..12: note = ROR1 epitope 10
AFPYCDETSSVP

SEQ ID NO: 209 - protein, 20 aa, synthetic construct
  REGION 1..20: note = ROR1 epitope 11
NVLCQTEYIFARSNPMILMR

SEQ ID NO: 210 - protein, 12 aa, synthetic construct
  REGION 1..12: note = ROR1 epitope 12
LKLPNCEDLPQP

SEQ ID NO: 211 - protein, 15 aa, synthetic construct
  REGION 1..15: note = ROR1 epitope 13
PESPEAANCIRIGIP

SEQ ID NO: 212 - protein, 14 aa, synthetic construct
  REGION 1..14: note = ROR1 epitope 14
VDYRGTVSVTKSGR

SEQ ID NO: 213 - protein, 14 aa, synthetic construct
  REGION 1..14: note = ROR1 epitope 15
SQYPHTHTFTALRF

SEQ ID NO: 214 - protein, 15 aa, synthetic construct
  REGION 1..15: note = ROR1 epitope 16
DSKEKNKMEILYILV

SEQ ID NO: 215 - protein, 937 aa, Homo sapiens
  REGION 1..937: note = MISC_FEATURE - ROR1 (Uniprot Q01973)
MHRPRRRGTRPPLLALLAALLLAARGAAAQETELSVSAELVPTSSWNISSELNKDSYLTLDEPMNNITTSLGQTAELHCKVSGNPPPTIRWFKNDAPVVQEPRRLSFRSTIYGSRLRIRNLDTTDTGYFQCVATNGKEVVSSTGVLFVKFGPPPTASPGYSDEYEEDGFCQPYRGIACARFIGNRTVYMESLHMQGEIENQITAAFTMIGTSSHLSDKCSQFAIPSLCHYAFPYCDETSSVPKPRDLCRDECEILENVLCQTEYIFARSNPMILMRLKLPNCEDLPQPESPEAANCIRIGIPMADPINKNHKCYNSTGVDYRGTVSVTKSGRQCQPWNSQYPHTHTFTALRFPELNGGHSYCRNPGNQKEAPWCFTLDENFKSDLCDIPACDSKDSKEKNKMEILYILVPSVAIPLAIALLFFFICVCRNNQKSSSAPVQRQPKHVRGQNVEMSMLNAYKPKSKAKELPLSAVRFMEELGECAFGKIYKGHLYLPGMDHAQLVAIKTLKDYNNPQQWTEFQQEASLMAELHHPNIVCLLGAVTQEQPVCMLFEYINQGDLHEFLIMRSPHSDVGCSSDEDGTVKSSLDHGDFLHIAIQIAAGMEYLSSHFFVHKDLAARNILIGEQLHVKISDLGLSREIYSADYYRVQSKSLLPIRWMPPEAIMYGKFSSDSDIWSFGVVLWEIFSFGLQPYYGFSNQEVIEMVRKRQLLPCSEDCPPRMYSLMTECWNEIPSRRPRFKDIHVRLRSWEGLSSHTSSTTPSGGNATTQTTSLSASPVSNLSNPRYPNYMFPSQGITPQGQIAGFIGPPIPQNQRFIPINGYPIPPGYAAFPAAHYQPTGPPRVIQHCPPPKSRSPSSASGSTSTGHVTSLPSSGSNQEANIPLLPHMSIPNHPGGMGITVFGNKSQKPYKIDSKQASLLGDANIHGHTESMISAEL

SEQ ID NO: 216 - protein, 937 aa, Macaca mulatta
  REGION 1..937: note = MISC_FEATURE - ROR1 (Uniprot F6RUP2)
MHRPRRRGTRPPLLALLAALLLAARGAAAQETELSVSAELVPTSSWNISSELNKDSYLTLDEPMNNITTSLGQTAELHCKVSGNPPPTIRWFKNDAPVVQEPRRLSFRSTIYGSRLRIRNLDTTDTGYFQCVATNGKEVVSSTGVLFVKFGPPPTASPGYSDEYEEDGFCQPYRGIACARFIGNRTVYMESLHMQGEIENQITAAFTMIGTSSHLSDKCSQFAIPSLCHYAFPYCDETSSVPKPRDLCRDECEILENVLCQTEYIFARSNPMILMRLKLPNCEDLPQPESPEAANCIRIGIPMADPINKNHKCYNSTGVDYRGTVSVTKSGRQCQPWNSQYPHTHTFTALRFPELNGGHSYCRNPGNQKEAPWCFTLDENFKSDLCDIPACDSKDSKEKNKMEILYILVPSVAIPLAIALLFFFICVCRNNQKSSSPPVQRQPKHVRGQNVEMSMLNAYKPKSKAKELPLSAVRFMEELGECAFGKIYKGHLYLPGMDHAQLVAIKTLKDYNNPQQWTEFQQEASLMAELHHPNIVCLLGAVTQEQPVCMLFEYMNQGDLHEFLIMRSPHSDVGCSSDEDGTVKSSLDHGDFLHIAIQIAAGMEYLSSHFFVHKDLAARNILIGEQLHVKISDLGLSREIYSADYYRVQSKSLLPIRWMPPEAIMYGKFSSDSDIWSFGVVLWEIFSFGLQPYYGFSNQEVIEMVRKRQLLPCSEDCPPRMYSLMTECWNEIPSRRPRFKDIHVRLRSWEGLSSHTSSTTPSGGNATTQTTSLSASPVSNLSNPRYPNYIFPSQGITPQGQIAGFIGPPIPQNQRFIPINGYPIPPGYAAFPAAHYQPTGPPRVIQHCPPPKSRSPSSASGSTSTGHVTSLPSSGSNQEANIPLLPHMSIPNHPGGMGITVFGNKSQKPYKIDAKQASLLGDANIHGHTESMISAEL

SEQ ID NO: 217 - protein, 923 aa, Homo sapiens
  REGION 1..923: note = MISC_FEATURE - ROR1 (Uniprot A0A2K5WTX7)
MLRTAHKLLYLILPLSFSLPFFFFSETELSVSAELVPTSSWNISSELNKDSYLTLDEPMNNITTSLGQTAELHCKVSGNPPPTIRWFKNDAPVVQEPRRLSFRSTIYGSRLRIRNLDTTDTGYFQCVATNGKEVVSSTGVLFVKFGKDEYEEDGFCQPYRGIACARFIGNRTVYMESLHMQGEIENQITAAFTMIGTSSHLSDKCSQFAIPSLCHYAFPYCDETSSVPKPRDLCRDECEILENVLCQTEYIFARSNPMILMRLKLPNCEDLPQPESPEAANCIRIGIPMADPINKNHKCYNSTGVDYRGTVSVTKSGRQCQPWNSQYPHTHTFTALRFPELNGGHSYCRNPGNQKEAPWCFTLDENFKSDLCDIPACDSKDSKEKNKMEILYILVPSVAIPLAIALLFFFICVCRNNQKSSSPPVQRQPKHVRGQNVEMSMLNAYKPKSKAKELPLSAVRFMEELGECAFGKIYKGHLYLPGMDHAQLVAIKTLKDYNNPQQWTEFQQEASLMAELHHPNIVCLLGAVTQEQPVCMLFEYMNQGDLHEFLIMRSPHSDVGCSSDEDGTVKSSLDHGDFLHIAIQIAAGMEYLSSHFFVHKDLAARNILIGEQLHVKISDLGLSREIYSADYYRVQSKSLLPIRWMPPEAIMYGKFSSDSDIWSFGVVLWEIFSFGLQPYYGFSNQEVIEMVRKRQLLPCSEDCPPRMYSLMTECWNEIPSRRPRFKDIHVRLRSWEGLSSHTSSTTPSGGNATTQTTSLSASPVSNLSNPRYPNYIFPSQGITPQGQIAGFIGPPIPQNQRFIPINGYPIPPGYAAFPAAHYQPTGPPRVIQHCPPPKSRSPSSASGSTSTGHVTSLPSSGSNQEANIPLLPHMSIPNHPGGMGITVFGNKSQKPYKIDAKQASLLGDANIHGHTESMISAEL

SEQ ID NO: 218 - protein, 882 aa, Homo sapiens
  REGION 1..882: note = MISC_FEATURE - ROR1 (Uniprot A0A2K5WTX4)
SYLTLDEPMNNITTSLGQTAELHCKVSGNPPPTIRWFKNDAPVVQEPRRLSFRSTIYGSRLRIRNLDTTDTGYFQCVATNGKEVVSSTGVLFVKFGPPPTASPGYSDEYEEDGFCQPYRGIACARFIGNRTVYMESLHMQGEIENQITAAFTMIGTSSHLSDKCSQFAIPSLCHYAFPYCDETSSVPKPRDLCRDECEILENVLCQTEYIFARSNPMILMRLKLPNCEDLPQPESPEAANCIRIGIPMADPINKNHKCYNSTGVDYRGTVSVTKSGRQCQPWNSQYPHTHTFTALRFPELNGGHSYCRNPGNQKEAPWCFTLDENFKSDLCDIPACDSKDSKEKNKMEILYILVPSVAIPLAIALLFFFICVCRNNQKSSSPPVQRQPKHVRGQNVEMSMLNAYKPKSKAKELPLSAVRFMEELGECAFGKIYKGHLYLPGMDHAQLVAIKTLKDYNNPQQWTEFQQEASLMAELHHPNIVCLLGAVTQEQPVCMLFEYMNQGDLHEFLIMRSPHSDVGCSSDEDGTVKSSLDHGDFLHIAIQIAAGMEYLSSHFFVHKDLAARNILIGEQLHVKISDLGLSREIYSADYYRVQSKSLLPIRWMPPEAIMYGKFSSDSDIWSFGVVLWEIFSFGLQPYYGFSNQEVIEMVRKRQLLPCSEDCPPRMYSLMTECWNEIPSRRPRFKDIHVRLRSWEGLSSHTSSTTPSGGNATTQTTSLSASPVSNLSNPRYPNYIFPSQGITPQGQIAGFIGPPIPQNQRFIPINGYPIPPGYAAFPAAHYQPTGPPRVIQHCPPPKSRSPSSASGSTSTGHVTSLPSSGSNQEANIPLLPHMSIPNHPGGMGITVFGNKSQKPYKIDAKQASLLGDANIHGHTESMISAEL

SEQ ID NO: 219 - protein, 937 aa, Homo sapiens
  REGION 1..937: note = MISC_FEATURE - ROR1 (Uniprot Q9Z139)
MHRPRRRGTRPPPLALLAALLLAARGADAQETELSVSAELVPTSSWNTSSEIDKGSYLTLDEPMNNITTSLGQTAELHCKVSGNPPPSIRWFKNDAPVVQEPRRISFRATNYGSRLRIRNLDTTDTGYFQCVATNGKKVVSTTGVLFVKFGPPPTASPGSSDEYEEDGFCQPYRGIACARFIGNRTVYMESLHMQGEIENQITAAFTMIGTSSHLSDKCSQFAIPSLCHYAFPYCDETSSVPKPRDLCRDECEVLENVLCQTEYIFARSNPMILMRLKLPNCEDLPQPESPEAANCIRIGIPMADPINKNHKCYNSTGVDYRGTVSVTKSGRQCQPWNSQYPHTHSFTALRFPELNGGHSYCRNPGNQKEAPWCFTLDENFKSDLCDIPACDSKDSKEKNKMEILYILVPSVAIPLAIAFLFFFICVCRNNQKSSSPPVQRQPKPVRGQNVEMSMLNAYKPKSKAKELPLSAVRFMEELGECTFGKIYKGHLYLPGMDHAQLVAIKTLKDYNNPQQWTEFQQEASLMAELHHPNIVCLLGAVTQEQPVCMLFEYMNQGDLHEFLIMRSPHSDVGCSSDEDGTVKSSLDHGDFLHIAIQIAAGMEYLSSHFFVHKDLAARNILIGEQLHVKISDLGLSREIYSADYYRVQSKSSLPIRWMPPEAIMYGKFSSDSDIWSFGVVLWEIFSFGLQPYYGFSNQEVIEMVRKRQLLPCSEDCPPRMYSLMTECWNEIPSRRPRFKDIHVRLRSWEGLSSHTSSTTPSGGNATTQTTSLSASPVSNLSNPRFPNYMFPSQGITPQGQIAGFIGPAIPQNQRFIPINGYPIPPGYAAFPAAHYQPAGPPRVIQHCPPPKSRSPSSASGSTSTGHVASLPSSGSNQEANVPLLPHMSIPNHPGGMGITVFGNKSQKPYKIDSKQSSLLGDSHIHGHTESMISAEV

(19) United States
(12) Patent Application Publication
(10) Pub. No.: US 2025/0242020 A1
(43) Pub. Date: Jul. 31, 2025

(54) ANTIBODIES AND CHIMERIC ANTIGEN RECEPTORS SPECIFIC FOR RECEPTOR TYROSINE KINASE LIKE ORPHAN RECEPTOR 1 (ROR1)

(71) Applicant: Juno Therapeutics, Inc., Seattle, WA (US)

(72) Inventors: Andreia COSTA, Seattle, WA (US); Rupesh AMIN, Seattle, WA (US); Jenna BAILEY, Seattle, WA (US); Samriti BEDI, Newcastle, WA (US); Brian BELMONT, Seattle, WA (US); Aye CHEN, Seattle, WA (US); Stephen Jacob GOLDFLESS, Seattle, WA (US); Eric JEFFERY, Seattle, WA (US); Yue JIANG, Los Angeles, CA (US); Yeonjoo OH, Seattle, WA (US); Madeline WILLIAMS, Seattle, WA (US); Collin HAUSKINS, Seattle, WA (US); Catherine SIERRA, Seattle, WA (US)

(73) Assignee: Juno Therapeutics, Inc., Seattle, WA (US)

(21) Appl. No.: 19/076,981

(22) Filed: Mar. 11, 2025

Related U.S. Application Data
(63) Continuation of application No. 17/421,365, filed on Jul. 7, 2021, now Pat. No. 12,268,741.
(60) Provisional application No. 62/798,456, filed on Jan. 29, 2019.

(51) Int. Cl. A61K 39/395; A61K 40/11; A61K 40/31; A61K 40/42; A61K 47/68; A61P 35/00; C12N 5/0783; C12N 5/10; C12N 15/62; C12N 15/63

(52) U.S. Cl. CPC A61K 39/39558; A61K 40/11; A61K 40/31; A61K 40/4202; A61K 47/6851; A61P 35/00; C12N 5/0636; C12N 5/10; C12N 15/62; C12N 15/63; A61K 2239/31; A61K 2239/38; A61K 2239/48; A61K 2239/49; A61K 2239/55

(57) ABSTRACT

Provided are receptor tyrosine kinase-like orphan receptor 1 (ROR1)-binding molecules, in particular, to human antibodies specific for ROR1, including antibody fragments. The present disclosure further relates to recombinant receptors, including chimeric antigen receptors (CARs) that contain such antibodies or fragments, and polynucleotides that encode the antibodies, antigen-binding fragments or receptors specific for ROR1. The disclosure further relates to genetically engineered cells, containing such ROR1-binding proteins and receptors, and related methods and uses thereof in adoptive cell therapy.

### CROSS-REFERENCE TO RELATED APPLICATIONS

This application is a continuation of U.S. application Ser. No. 17/421,365, filed Jan. 7, 2021, which is a National Stage application under 35 U.S.C. § 371 of International Application No. PCT/US2020/015489, filed on Jan. 28, 2020, which claims priority from U.S. provisional application No. 62/798,456, filed Jan. 29, 2019, entitled “ANTIBODIES AND CHIMERIC ANTIGEN RECEPTORS SPECIFIC FOR RECEPTOR TYROSINE KINASE LIKE ORPHAN RECEPTOR 1 (ROR1),” the contents of which are incorporated by reference in their entirety.

### INCORPORATION BY REFERENCE OF SEQUENCE LISTING

The present application is being filed along with a Sequence Listing in electronic format. The Sequence Listing is provided as a file entitled 735042017701SeqList.txt, created Mar. 10, 2025, which is 321,547 bytes in size. The information in the electronic format of the Sequence Listing is incorporated by reference in its entirety.

### FIELD

The present disclosure relates in some aspects to receptor tyrosine kinase-like orphan receptor 1 (ROR1)-binding molecules, in particular, to human antibodies specific for ROR1, including antibody fragments. The present disclosure further relates to recombinant receptors, including chimeric antigen receptors (CARs) that contain such antibodies or fragments, and polynucleotides that encode the antibodies, antigen-binding fragments or receptors specific for ROR1. The disclosure further relates to genetically engineered cells, containing such ROR1-binding proteins and receptors, and related methods and uses thereof in adoptive cell therapy.

### BACKGROUND

Receptor tyrosine kinase-like orphan receptor 1 (ROR1) is a transmembrane receptor expressed during embryogenesis, but typically not in normal adult cells. ROR1, however, is expressed in the context of a variety of different cancers, and in some cases, involved in cell signaling to promote tumor cell survival. Based on its expression, ROR1 could be a tumor-specific and/or tumor-associated target for therapy. ROR1-binding molecules, receptors and cells expressing such molecules are available. Improved ROR1-binding molecules and engineered ROR1-binding receptor-expressing cells are needed. Provided are embodiments that meet such needs.

### SUMMARY

Provided herein is an anti-ROR1 antibody or antigen-binding fragment thereof, containing: a heavy chain variable (V_{H}) region, and a light chain variable (V_{L}) region, wherein: (i) the V_{H} region contains a heavy chain complementarity determining region 1 (CDR-H1) containing the sequence set forth in SEQ ID NO: 67, 82 or 52, a heavy chain complementarity determining region 2 (CDR-H2) containing the sequence set forth in SEQ ID NO: 71, 86, 56 or 97, and a heavy chain complementarity determining region 3 (CDR-H3) containing the sequence set forth in SEQ ID NO: 73, 88, 58 or 99, and the V_{L} region contains a light chain complementarity determining region 1 (CDR-L1) containing the sequence set forth in SEQ ID NO: 75, 90 or 60, a light chain complementarity determining region 2 (CDR-L2) containing the sequence set forth in SEQ ID NO: 77, 92 or 62; and a light chain complementarity determining region 3 (CDR-L3) containing the sequence set forth in SEQ ID NO: 79, 94 or 64; or (ii) the V_{H} region contains a CDR-H1 containing the sequence set forth in SEQ ID NO: 65, 80 or 50, a CDR-H2 containing the sequence set forth in SEQ ID NO: 69, 84, 54 or 95, and a CDR-H3 containing the sequence set forth in SEQ ID NO: 73, 88, 58 or 99, and the V_{L} region contains a CDR-L1 containing the sequence set forth in SEQ ID NO: 75, 90 or 60, a CDR-L2 containing the sequence set forth in SEQ ID NO: 77, 92 or 62; and a CDR-L3 containing the sequence set forth in SEQ ID NO: 79, 94 or 64; or (iii) the V_{H} region contains a CDR-H1 containing the sequence set forth in SEQ ID NO: 66, 81 or 51, a CDR-H2 containing the sequence set forth in SEQ ID NO: 70, 85, 55 or 96, and a CDR-H3 containing the sequence set forth in SEQ ID NO: 73, 88, 58 or 99, and the V_{L} region contains a CDR-L1 containing the sequence set forth in SEQ ID NO: 75, 90 or 60, a CDR-L2 containing the sequence set forth in SEQ ID NO: 77, 92 or 62; and a CDR-L3 containing the sequence set forth in SEQ ID NO: 79, 94 or 64; or (iv) the V_{H} region contains a CDR-H1 containing the sequence set forth in SEQ ID NO: 68, 83 or 53, a CDR-H2 containing the sequence set forth in SEQ ID NO: 72, 87, 57 or 98, and a CDR-H3 containing the sequence set forth in SEQ ID NO: 74, 89, 59 or 100, and the V_{L} region contains a CDR-L1 containing the sequence set forth in SEQ ID NO: 76, 91 or 61, a CDR-L2 containing the sequence set forth in SEQ ID NO: 78, 93 or 63; and a CDR-L3 containing the sequence set forth in SEQ ID NO: 79, 94 or 64.

Also provided herein is an anti-ROR1 antibody or antigen-binding fragment thereof, containing: a heavy chain variable (V_{H}) region, and a light chain variable (V_{L}) region, wherein: (i) the V_{H} region contains a heavy chain complementarity determining region 1 (CDR-H1), a heavy chain complementarity determining region 2 (CDR-H2) and a heavy chain complementarity determining region 3 (CDR-H3) containing the sequence set forth in SEQ ID NOS:67, 71 and 73, respectively, and the V_{L} region contains a light chain complementarity determining region 1 (CDR-L1), a light chain complementarity determining region 2 (CDR-L2) and a light chain complementarity determining region 3 (CDR-L3) containing the sequence set forth in SEQ ID NOS:75, 77 and 79, respectively; the V_{H} region contains a CDR-H1, a CDR-H2 and a CDR-H3 containing the sequence set forth in SEQ ID NOS:82, 86 and 88, respectively, and the V_{L} region contains a CDR-L1, a CDR-L2 and a CDR-L3 containing the sequence set forth in SEQ ID NOS:90, 92 and 94, respectively; the V_{H} region contains a CDR-H1, a CDR-H2 and a CDR-H3 containing the sequence set forth in SEQ ID NOS:52, 56 and 58, respectively, and the V_{L} region contains a CDR-L1, a CDR-L2 and a CDR-L3 containing the sequence set forth in SEQ ID NOS:60, 62 and 64, respectively; or the V_{H} region contains a CDR-H1, a CDR-H2 and a CDR-H3 containing the sequence set forth in SEQ ID NOS:52, 97 and 99, respectively, and the V_{L} region contains a CDR-L1, a CDR-L2 and a CDR-L3 containing the sequence set forth in SEQ ID NOS:60, 62 and 64, respectively; (ii) the V_{H} region contains a CDR-H1, a CDR-H2 and a CDR-H3 containing the sequence set forth in SEQ ID NOS:65, 69 and 73, respectively, and the V_{L} region contains a CDR-L1, a CDR-L2 and a CDR-L3 containing the sequence set forth in SEQ ID NOS:75, 77 and 79, respectively; the V_{H} region contains a CDR-H1, a CDR-H2 and a CDR-H3 containing the sequence set forth in SEQ ID NOS:80, 84 and 88, respectively, and the V_{L} region contains a CDR-L1, a CDR-L2 and a CDR-L3 containing the sequence set forth in SEQ ID NOS:90, 92 and 94, respectively; the V_{H} region contains a CDR-H1, a CDR-H2 and a CDR-H3 containing the sequence set forth in SEQ ID NOS:50, 54 and 58, respectively, and the V_{L} region contains a CDR-L1, a CDR-L2 and a CDR-L3 containing the sequence set forth in SEQ ID NOS:60, 62 and 64, respectively; or the V_{H} region contains a CDR-H1, a CDR-H2 and a CDR-H3 containing the sequence set forth in SEQ ID NOS:50, 95 and 99, respectively, and the V_{L} region contains a CDR-L1, a CDR-L2 and a CDR-L3 containing the sequence set forth in SEQ ID NOS:60, 62 and 64, respectively; (iii) the V_{H} region contains a CDR-H1, a CDR-H2 and a CDR-H3 containing the sequence set forth in SEQ ID NOS:66, 70 and 73, respectively, and the V_{L} region contains a CDR-L1, a CDR-L2 and a CDR-L3 containing the sequence set forth in SEQ ID NOS:75, 77 and 79, respectively; the V_{H} region contains a CDR-H1, a CDR-H2 and a CDR-H3 containing the sequence set forth in SEQ ID NOS:81, 85 and 88, respectively, and the V_{L} region contains a CDR-L1, a CDR-L2 and a CDR-L3 containing the sequence set forth in SEQ ID NOS:90, 92 and 94, respectively; the V_{H} region contains a CDR-H1, a CDR-H2 and a CDR-H3 containing the sequence set forth in SEQ ID NOS:51, 55 and 58, respectively, and the V_{L} region contains a CDR-L1, a CDR-L2 and a CDR-L3 containing the sequence set forth in SEQ ID NOS:60, 62 and 64, respectively; or the V_{H} region contains a CDR-H1, a CDR-H2 and a CDR-H3 containing the sequence set forth in SEQ ID NOS:51, 96 and 99, respectively, and the V_{L} region contains a CDR-L1, a CDR-L2 and a CDR-L3 containing the sequence set forth in SEQ ID NOS:60, 62 and 64, respectively; (iv) the V_{H} region contains a CDR-H1, a CDR-H2 and a CDR-H3 containing the sequence set forth in SEQ ID NOS:68, 72 and 74, respectively, and the V_{L} region contains a CDR-L1, a CDR-L2 and a CDR-L3 containing the sequence set forth in SEQ ID NOS:76, 78 and 79, respectively; or the V_{H} region contains a CDR-H1, a CDR-H2 and a CDR-H3 containing the sequence set forth in SEQ ID NOS:83, 87 and 89, respectively, and the V_{L} region contains a CDR-L1, a CDR-L2 and a CDR-L3 containing the sequence set forth in SEQ ID NOS:91, 93 and 94, respectively; the V_{H} region contains a CDR-H1, a CDR-H2 and a CDR-H3 containing the sequence set forth in SEQ ID NOS:53, 57 and 59, respectively, and the V_{L} region contains a CDR-L1, a CDR-L2 and a CDR-L3 containing the sequence set forth in SEQ ID NOS:60, 63 and 64, respectively; or the V_{H} region contains a CDR-H1, a CDR-H2 and a CDR-H3 containing the sequence set forth in SEQ ID NOS:53, 98 and 100, respectively, and the V_{L} region contains a CDR-L1, a CDR-L2 and a CDR-L3 containing the sequence set forth in SEQ ID NOS:61, 63 and 64, respectively.

In some of any such embodiments: (i) the V_{H} region contains a CDR-H1, a CDR-H2 and a CDR-H3 containing the sequence set forth in SEQ ID NOS:67, 71 and 73, respectively, and the V_{L} region contains a CDR-L1, a CDR-L2 and a CDR-L3 containing the sequence set forth in SEQ ID NOS:75, 77 and 79, respectively; (ii) the V_{H} region contains a CDR-H1, a CDR-H2 and a CDR-H3 containing the sequence set forth in SEQ ID NOS:65, 69 and 73, respectively, and the V_{L} region contains a CDR-L1, a CDR-L2 and a CDR-L3 containing the sequence set forth in SEQ ID NOS:75, 77 and 79, respectively; (iii) the V_{H} region contains a CDR-H1, a CDR-H2 and a CDR-H3 containing the sequence set forth in SEQ ID NOS:66, 70 and 73, respectively, and the V_{L} region contains a CDR-L1, a CDR-L2 and a CDR-L3 containing the sequence set forth in SEQ ID NOS:75, 77 and 79, respectively; or (iv) the V_{H} region contains a CDR-H1, a CDR-H2 and a CDR-H3 containing the sequence set forth in SEQ ID NOS:68, 72 and 74, respectively, and the V_{L} region contains a CDR-L1, a CDR-L2 and a CDR-L3 containing the sequence set forth in SEQ ID NOS:76, 78 and 79, respectively.

In some of any such embodiments: (i) the V_{H} region contains a CDR-H1, a CDR-H2 and a CDR-H3 containing the sequence set forth in SEQ ID NOS:82, 86 and 88, respectively, and the V_{L} region contains a CDR-L1, a CDR-L2 and a CDR-L3 containing the sequence set forth in SEQ ID NOS:90, 92 and 94, respectively; (ii) the V_{H} region contains a CDR-H1, a CDR-H2 and a CDR-H3 containing the sequence set forth in SEQ ID NOS:80, 84 and 88, respectively, and the V_{L} region contains a CDR-L1, a CDR-L2 and a CDR-L3 containing the sequence set forth in SEQ ID NOS:90, 92 and 94, respectively; (iii) the V_{H} region contains a CDR-H1, a CDR-H2 and a CDR-H3 containing the sequence set forth in SEQ ID NOS:81, 85 and 88, respectively, and the V_{L} region contains a CDR-L1, a CDR-L2 and a CDR-L3 containing the sequence set forth in SEQ ID NOS:90, 92 and 94, respectively; or (iv) the V_{H} region contains a CDR-H1, a CDR-H2 and a CDR-H3 containing the sequence set forth in SEQ ID NOS:83, 87 and 89, respectively, and the V_{L} region contains a CDR-L1, a CDR-L2 and a CDR-L3 containing the sequence set forth in SEQ ID NOS:91, 93 and 94, respectively.

In some of any embodiments, the V_{H} region comprises a CDR-H1, a CDR-H2 and a CDR-H3 comprising the sequence set forth in SEQ ID NOS:67, 71 and 73, respectively, and the V_{L} region comprises a CDR-L1, a CDR-L2 and a CDR-L3 comprising the sequence set forth in SEQ ID NOS:75, 77 and 79, respectively. In some of any embodiments, the V_{H} region comprises a CDR-H1, a CDR-H2 and a CDR-H3 comprising the sequence set forth in SEQ ID NOS:82, 86 and 88, respectively, and the V_{L} region comprises a CDR-L1, a CDR-L2 and a CDR-L3 comprising the sequence set forth in SEQ ID NOS:90, 92 and 94, respectively. In some of any such embodiments, the V_{H} region comprises a CDR-H1, a CDR-H2 and a CDR-H3 comprising the sequence set forth in SEQ ID NOS:52, 56 and 58, respectively, and the V_{L} region comprises a CDR-L1, a CDR-L2 and a CDR-L3 comprising the sequence set forth in SEQ ID NOS:60, 62 and 64, respectively. In some of any such embodiments, the V_{H} region comprises a CDR-H1, a CDR-H2 and a CDR-H3 comprising the sequence set forth in SEQ ID NOS:52, 97 and 99, respectively, and the V_{L} region comprises a CDR-L1, a CDR-L2 and a CDR-L3 comprising the sequence set forth in SEQ ID NOS:60, 62 and 64, respectively.

Also provided herein is an anti-ROR1 antibody or antigen-binding fragment thereof, containing: a heavy chain variable (V_{H}) region and a light chain variable (V_{L}) region, wherein: the V_{H} region contains a heavy chain complementarity determining region 1 (CDR-H1), a heavy chain complementarity determining region 2 (CDR-H2) and a heavy chain complementarity determining region 3 (CDR-H3) contained within SEQ ID NO: 112, 121, 103 or 130, and the V_{L} region contains a light chain complementarity determining region 1 (CDR-L1), a light chain complementarity determining region 2 (CDR-L2) and a light chain complementarity determining region 3 (CDR-L3) contained within SEQ ID NO: 115, 124 or 106.

Also provided herein is an anti-ROR1 antibody or antigen-binding fragment thereof, containing: a heavy chain variable (V_{H}) region and a light chain variable (V_{L}) region, wherein: the V_{H} region contains a heavy chain complementarity determining region 1 (CDR-H1), a heavy chain complementarity determining region 2 (CDR-H2) and a heavy chain complementarity determining region 3 (CDR-H3) contained within SEQ ID NO:112, and the V_{L} region contains a light chain complementarity determining region 1 (CDR-L1), a light chain complementarity determining region 2 (CDR-L2) and a light chain complementarity determining region 3 (CDR-L3) contained within SEQ ID NO:115; the V_{H} region contains a CDR-H1, a CDR-H2 and a CDR-H3 contained within SEQ ID NO:121, and the V_{L} region contains a CDR-L1, a CDR-L2 and a CDR-L3 contained within SEQ ID: NO 124; the V_{H} region contains a CDR-H1, a CDR-H2 and a CDR-H3 contained within SEQ ID NO:103, and the V_{L} region contains a CDR-L1, a CDR-L2 and a CDR-L3 contained within SEQ ID: NO 106; or the V_{H} region contains a CDR-H1, a CDR-H2 and a CDR-H3 contained within SEQ ID NO:130, and the V_{L} region contains a CDR-L1, a CDR-L2 and a CDR-L3 contained within SEQ ID: NO 106.

In some of any such embodiments, the V_{H} region contains a CDR-H1, a CDR-H2 and a CDR-H3 contained within SEQ ID NO:112, and the V_{L} region contains a CDR-L1, a CDR-L2 and a CDR-L3 contained within SEQ ID: NO 115. In some of any such embodiments, the V_{H} region contains a CDR-H1, a CDR-H2 and a CDR-H3 contained within SEQ ID NO:121, and the V_{L} region contains a CDR-L1, a CDR-L2 and a CDR-L3 contained within SEQ ID: NO 124. In some of any embodiments, the V_{H} region comprises a CDR-H1, a CDR-H2 and a CDR-H3 contained within SEQ ID NO:103, and the V_{L} region comprises a CDR-L1, a CDR-L2 and a CDR-L3 contained within SEQ ID: NO 106. In some of any embodiments, the V_{H} region comprises a CDR-H1, a CDR-H2 and a CDR-H3 contained within SEQ ID NO:130, and the V_{L} region comprises a CDR-L1, a CDR-L2 and a CDR-L3 contained within SEQ ID: NO 106.

In some of any such embodiments, the V_{H} region is or contains an amino acid sequence having at least at or about 85%, 86%, 87%, 88%, 89%, 90%, 91%, 92%, 93%, 94%, 95%, 96%, 97%, 98% or 99% identity to SEQ ID NO: 112, 121, 103 or 130, and the V_{L} region is or contains an amino acid sequence having at least at or about 85%, 86%, 87%, 88%, 89%, 90%, 91%, 92%, 93%, 94%, 95%, 96%, 97%, 98% or 99% identity to SEQ ID NO: 115, 124 or 106. In some of any such embodiments, the V_{H} region is or contains an amino acid sequence having at least at or about 85%, 86%, 87%, 88%, 89%, 90%, 91%, 92%, 93%, 94%, 95%, 96%, 97%, 98% or 99% identity to SEQ ID NO:112, and the V_{L} region is or contains an amino acid sequence having at least at or about 85%, 86%, 87%, 88%, 89%, 90%, 91%, 92%, 93%, 94%, 95%, 96%, 97%, 98% or 99% identity to SEQ ID NO: 115; the V_{H} region is or contains an amino acid sequence having at least at or about 85%, 86%, 87%, 88%, 89%, 90%, 91%, 92%, 93%, 94%, 95%, 96%, 97%, 98% or 99% identity to SEQ ID NO:121, and the V_{L} region is or contains an amino acid sequence having at least at or about 85%, 86%, 87%, 88%, 89%, 90%, 91%, 92%, 93%, 94%, 95%, 96%, 97%, 98% or 99% identity to SEQ ID NO:124; the V_{H} region is or contains an amino acid sequence having at least at or about 85%, 86%, 87%, 88%, 89%, 90%, 91%, 92%, 93%, 94%, 95%, 96%, 97%, 98% or 99% identity to SEQ ID NO:103, and the V_{L} region is or contains an amino acid sequence having at least at or about 85%, 86%, 87%, 88%, 89%, 90%, 91%, 92%, 93%, 94%, 95%, 96%, 97%, 98% or 99% identity to SEQ ID NO:106; or the V_{H} region is or contains an amino acid sequence having at least at or about 85%, 86%, 87%, 88%, 89%, 90%, 91%, 92%, 93%, 94%, 95%, 96%, 97%, 98% or 99% identity to SEQ ID NO:130, and the V_{L} region is or contains an amino acid sequence having at least at or about 85%, 86%, 87%, 88%, 89%, 90%, 91%, 92%, 93%, 94%, 95%, 96%, 97%, 98% or 99% identity to SEQ ID NO:106.

In some of any such embodiments, the V_{H} region is or contains an amino acid sequence having at least at or about 85%, 86%, 87%, 88%, 89%, 90%, 91%, 92%, 93%, 94%, 95%, 96%, 97%, 98% or 99% identity to SEQ ID NO:112, and the V_{L} region is or contains an amino acid sequence having at least at or about 85%, 86%, 87%, 88%, 89%, 90%, 91%, 92%, 93%, 94%, 95%, 96%, 97%, 98% or 99% identity to SEQ ID NO: 115. In some of any such embodiments, the V_{H} region is or contains an amino acid sequence having at least at or about 85%, 86%, 87%, 88%, 89%, 90%, 91%, 92%, 93%, 94%, 95%, 96%, 97%, 98% or 99% identity to SEQ ID NO:121, and the V_{L} region is or contains an amino acid sequence having at least at or about 85%, 86%, 87%, 88%, 89%, 90%, 91%, 92%, 93%, 94%, 95%, 96%, 97%, 98% or 99% identity to SEQ ID NO:124. In some of any embodiments, the V_{H} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO:103, and the V_{L} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO:106. In some of any embodiments, the V_{H} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO:130, and the V_{L} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO:106.

Also provided herein is an anti-ROR1 antibody or antigen-binding fragment thereof, containing: a heavy chain variable (V_{H}) region and a light chain variable (V_{L}) region, wherein: the V_{H} region is or contains an amino acid sequence having at least at or about 85%, 86%, 87%, 88%, 89%, 90%, 91%, 92%, 93%, 94%, 95%, 96%, 97%, 98% or 99% identity to SEQ ID NO: 112, 121, 103 or 130, and the V_{L} region is or contains an amino acid sequence having at least at or about 85%, 86%, 87%, 88%, 89%, 90%, 91%, 92%, 93%, 94%, 95%, 96%, 97%, 98% or 99% identity to SEQ ID NO: 115, 124 or 106.

Also provided herein is an anti-ROR1 antibody or antigen-binding fragment thereof, containing: a heavy chain variable (V_{H}) region and a light chain variable (V_{L}) region, wherein: the V_{H} region is or contains an amino acid sequence having at least at or about 85%, 86%, 87%, 88%, 89%, 90%, 91%, 92%, 93%, 94%, 95%, 96%, 97%, 98% or 99% identity to SEQ ID NO:112, and the V_{L} region is or contains an amino acid sequence having at least at or about 85%, 86%, 87%, 88%, 89%, 90%, 91%, 92%, 93%, 94%, 95%, 96%, 97%, 98% or 99% identity to SEQ ID NO: 115; the V_{H} region is or contains an amino acid sequence having at least at or about 85%, 86%, 87%, 88%, 89%, 90%, 91%, 92%, 93%, 94%, 95%, 96%, 97%, 98% or 99% identity to SEQ ID NO:121, and the V_{L} region is or contains an amino acid sequence having at least at or about 85%, 86%, 87%, 88%, 89%, 90%, 91%, 92%, 93%, 94%, 95%, 96%, 97%, 98% or 99% identity to SEQ ID NO:124; the V_{H} region is or contains an amino acid sequence having at least at or about 85%, 86%, 87%, 88%, 89%, 90%, 91%, 92%, 93%, 94%, 95%, 96%, 97%, 98% or 99% identity to SEQ ID NO:103, and the V_{L} region is or contains an amino acid sequence having at least at or about 85%, 86%, 87%, 88%, 89%, 90%, 91%, 92%, 93%, 94%, 95%, 96%, 97%, 98% or 99% identity to SEQ ID NO:106; or the V_{H} region is or contains an amino acid sequence having at least at or about 85%, 86%, 87%, 88%, 89%, 90%, 91%, 92%, 93%, 94%, 95%, 96%, 97%, 98% or 99% identity to SEQ ID NO:130, and the V_{L} region is or contains an amino acid sequence having at least at or about 85%, 86%, 87%, 88%, 89%, 90%, 91%, 92%, 93%, 94%, 95%, 96%, 97%, 98% or 99% identity to SEQ ID NO:106.

In some of any such embodiments, the V_{H} region is or contains an amino acid sequence having at least at or about 85%, 86%, 87%, 88%, 89%, 90%, 91%, 92%, 93%, 94%, 95%, 96%, 97%, 98% or 99% identity to SEQ ID NO:112, and the V_{L} region is or contains an amino acid sequence having at least at or about 85%, 86%, 87%, 88%, 89%, 90%, 91%, 92%, 93%, 94%, 95%, 96%, 97%, 98% or 99% identity to SEQ ID NO: 115. In some of any such embodiments, the V_{H} region is or contains an amino acid sequence having at least at or about 85%, 86%, 87%, 88%, 89%, 90%, 91%, 92%, 93%, 94%, 95%, 96%, 97%, 98% or 99% identity to SEQ ID NO:121, and the V_{L} region is or contains an amino acid sequence having at least at or about 85%, 86%, 87%, 88%, 89%, 90%, 91%, 92%, 93%, 94%, 95%, 96%, 97%, 98% or 99% identity to SEQ ID NO:124.

In some of any such embodiments: the V_{H} region is or contains the sequence set forth in SEQ ID NO: 112, 121, 103 or 130, and the V_{L} region is or contains the sequence set forth in SEQ ID NO: 115, 124 or 106. In some of any such embodiments: the V_{H} region and the V_{L} region are or contain the sequence set forth in SEQ ID NOS: 112 and 115, respectively; the V_{H} region and the V_{L} region are or contain the sequence set forth in SEQ ID NOS: 121 and 124, respectively; the V_{H} region and the V_{L} region are or contain the sequence set forth in SEQ ID NOS: 103 and 106, respectively; or the V_{H} region and the V_{L} region are or contain the sequence set forth in SEQ ID NOS: 130 and 106, respectively.

Also provided herein is an anti-ROR1 antibody or antigen-binding fragment thereof, containing: a heavy chain variable (V_{H}) region and a light chain variable (V_{L}) region, wherein: the V_{H} region is or contains the sequence set forth in SEQ ID NO: 112, 121, 103 or 130, and the V_{L} region is or contains the sequence set forth in SEQ ID NO: 115, 124 or 106.

Also provided herein is an anti-ROR1 antibody or antigen-binding fragment thereof, containing: a heavy chain variable (V_{H}) region and a light chain variable (V_{L}) region, wherein: the V_{H} region and the V_{L} region are or contain the sequence set forth in SEQ ID NOS: 112 and 115, respectively; the V_{H} region and the V_{L} region are or contain the sequence set forth in SEQ ID NOS: 121 and 124, respectively; the V_{H} region and the V_{L} region are or contain the sequence set forth in SEQ ID NOS: 103 and 106, respectively; or the V_{H} region and the V_{L} region are or contain the sequence set forth in SEQ ID NOS: 130 and 106, respectively.

In some of any such embodiments, the V_{H} region and the V_{L} region are or contain the sequence set forth in SEQ ID NOS: 112 and 115, respectively. In some of any such embodiments, the V_{H} region and the V_{L} region are or contain the sequence set forth in SEQ ID NOS:121 and 124, respectively. In some of any embodiments, the V_{H} region and the V_{L} region are or comprise the sequence set forth in SEQ ID NOS: 103 and 106, respectively. In some of any embodiments, the V_{H} region and the V_{L} region are or comprise the sequence set forth in SEQ ID NOS:130 and 106, respectively.

In some of any such embodiments, the V_{H} region is or contains the amino acid sequence encoded by SEQ ID NO: 110, 119, 101 or 128 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 110, 119, 101 or 128, and the V_{L} region is or contains the amino acid sequence encoded by SEQ ID NO: 113, 122 or 104, or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 113, 122 or 104. In some of any such embodiments, the V_{H} region is or contains the amino acid sequence encoded by SEQ ID NO: 111, 120, 102 or 129, and the V_{L} region is or contains the amino acid sequence encoded by SEQ ID NO: 114, 123, 105 or 131. In some of any such embodiments, the V_{H} region is or contains the amino acid sequence encoded by SEQ ID NO: 110 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 110, and the V_{L} region is or contains the amino acid sequence encoded by SEQ ID NO: 113, or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 113. In some of any such embodiments, the V_{H} region is or contains the amino acid sequence encoded by SEQ ID NO: 111, and the V_{L} region is or contains the amino acid sequence encoded by SEQ ID NO: 114. In some of any such embodiments, the V_{H} region is or contains the amino acid sequence encoded by SEQ ID NO: 119 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 119, and the V_{L} region is or contains the amino acid sequence encoded by SEQ ID NO: 122, or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 122. In some of any such embodiments, the V_{H} region is or contains the amino acid sequence encoded by SEQ ID NO: 120, and the V_{L} region is or contains the amino acid sequence encoded by SEQ ID NO: 123.

In some of any embodiments, the antibody is a full length antibody. In some of any embodiments, the antibody is an antigen-binding fragment. In some of any such embodiments, said anti-ROR1 antibody or antigen-binding fragment thereof is isolated. In some of any such embodiments, said anti-ROR1 antibody or antigen-binding fragment thereof is recombinant. In some of any such embodiments, at least a portion of the V_{H} region and the V_{L} region is human or is from a human protein.

In some of any such embodiments, the antigen-binding fragment thereof is or contains a single chain fragment. In some of any such embodiments, the antigen-binding fragment thereof is or contains a single chain Fv (scFv).

In some of any such embodiments, the V_{H} region is amino-terminal to the V_{L} region. In some of any such embodiments, the V_{H} region is carboxy-terminal to the V_{L} region. In some of any such embodiments, the V_{H} region and the V_{L} region are joined by a flexible linker. In some of any such embodiments, the flexible linker contains the sequence set forth in SEQ ID NO:41.

In some of any such embodiments, the scFv is or contains the sequence set forth in SEQ ID NO: 118, 127, 109 or 134, or an amino acid sequence having at least at or about 85%, 86%, 87%, 88%, 89%, 90%, 91%, 92%, 93%, 94%, 95%, 96%, 97%, 98% or 99% sequence identity to SEQ ID NO: 118, 127, 109 or 134. In some of any such embodiments, the scFv is or contains the sequence set forth in SEQ ID NO: 118. In some of any such embodiments, the scFv is or contains the sequence set forth in SEQ ID NO: 127. In some of any embodiments, the scFv is or comprises the sequence set forth in SEQ ID NO: 109. In some of any embodiments, the scFv is or comprises the sequence set forth in SEQ ID NO: 134.

In some of any such embodiments, the scFv is or contains the amino acid sequence encoded by SEQ ID NO: 116, 125, 107 or 132 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 116, 125, 107 or 132. In some of any such embodiments, the scFv is or contains the amino acid sequence encoded by SEQ ID NO: 117, 126, 108 or 133. In some of any such embodiments, the scFv is or contains the amino acid sequence encoded by SEQ ID NO: 116 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 116. In some of any such embodiments, the scFv is or contains the amino acid sequence encoded by SEQ ID NO: 117. In some of any such embodiments, the scFv is or contains the amino acid sequence encoded by SEQ ID NO: 125 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 125. In some of any such embodiments, the scFv is or contains the amino acid sequence encoded by SEQ ID NO: 126.

In some of any such embodiments, the anti-ROR1 antibody or fragment further contains at least a portion of an immunoglobulin constant region or a variant thereof. In some of any such embodiments, the portion of an immunoglobulin constant region contains at least a portion of a hinge region or a variant thereof. In some of any such embodiments, the at least a portion of an immunoglobulin constant region or a variant thereof contains at least a portion of a C_{H}2 region or a variant thereof. In some of any such embodiments, the at least a portion of an immunoglobulin constant region or a variant thereof contains at least a portion of a C_{H}3 region or a variant thereof. In some of any such embodiments, the at least a portion of an immunoglobulin constant region or a variant thereof contains at least a portion of a C_{H}2 region and/or a C_{H}3 region or a variant thereof. In some of any such embodiments, the at least a portion of an immunoglobulin constant region or a variant thereof is human or from a human protein or a variant thereof.

In some of any such embodiments, said anti-ROR1 antibody or antigen-binding fragment thereof specifically binds to a Receptor tyrosine kinase-like orphan receptor 1 (ROR1) protein. In some of any such embodiments, said anti-ROR1 antibody or antigen-binding fragment thereof specifically binds to a human ROR1 protein. In some of any such embodiments, the human ROR1 protein contains an amino acid sequence set forth in SEQ ID NO: 144, 145 or 146.

In some of any embodiments, the anti-ROR1 antibody or antigen-binding fragment thereof specifically binds to an epitope consisting of the sequence set forth in SEQ ID NO:199 or an epitope present within the sequence set forth in SEQ ID NO:199. In some of any embodiments, the antibody or antigen-binding fragment thereof further binds to one or more epitopes consisting of a sequence selected from among any one of SEQ ID NOS: 200-214 or an epitope present within a sequence selected from among any one of SEQ ID NOS: 200-214. In some of any embodiments, the one or more epitopes comprises a conformational epitope.

In some of any such embodiments, said anti-ROR1 antibody or antigen-binding fragment thereof does not bind to, is not cross-reactive to, or binds at a lower level or degree or affinity to a Receptor tyrosine kinase-like orphan receptor 2 (ROR2) protein. In some of any such embodiments, said anti-ROR1 antibody or antigen-binding fragment thereof does not bind to, is not cross-reactive to, or binds at a lower level or degree or affinity to a human ROR2 protein. In some of any such embodiments, the extent, level or degree or affinity of binding of said anti-ROR1 antibody or antigen-binding fragment thereof to a human ROR2 is at least at or about 75%, 80%, 90%, 95% or 99% less than the extent, level or degree or affinity of binding to a human ROR1 protein.

In some of any embodiments, the antibody or antigen-binding fragment thereof binds to human ROR1 protein with an equilibrium dissociation constant (K_{D}) of from about 1×10⁻¹¹ M to about 1×10⁻⁷ M. In some of any embodiments, the antibody or antigen-binding fragment thereof binds to human ROR1 protein with an equilibrium dissociation constant (K_{D}) of from about 1×10⁸ M to about 1×10⁻⁷ M. In some of any embodiments, the antibody or antigen-binding fragment thereof binds to human ROR1 protein with an equilibrium dissociation constant (K_{D}) of from about 5×10⁻¹¹ M to about 1×10⁻¹⁰ M.

In some of any embodiments, the antibody or antigen-binding fragment thereof binds to human ROR1 protein with an dissociation rate constant (k_{d} or k_{off}) of from about 1×10⁻⁵ 1/s to about 1×10² 1/s. In some of any embodiments, the antibody or antigen-binding fragment thereof binds to human ROR1 protein with an dissociation rate constant (k_{d} or k_{off}) of from about 1×10⁻³ 1/s to about 1×10⁻² 1/s. In some of any embodiments, the antibody or antigen-binding fragment thereof binds to human ROR1 protein with an dissociation rate constant (k_{d} or k_{off}) of from about 1×10⁻⁵ 1/s to about 1×10⁻⁴ 1/s.

Also provided herein is a single chain cell-surface protein, containing the anti-ROR1 antibody or antigen-binding fragment thereof of any of the embodiments provided herein.

Also provided herein is a conjugate, containing the anti-ROR1 antibody or antigen-binding fragment thereof of any of the embodiments provided herein and a heterologous molecule or moiety. In some of any such embodiments, the heterologous molecule or moiety is a therapeutic moiety.

Also provided herein is an anti-ROR1 chimeric antigen receptor (CAR) containing an extracellular antigen-binding domain containing the anti-ROR1 antibody or antigen-binding fragment thereof of any of the embodiments provided herein and an intracellular signaling region.

Also provided herein is an anti-ROR1 chimeric antigen receptor (CAR) containing an extracellular antigen-binding domain containing the anti-ROR1 antibody or antigen-binding fragment thereof of any of the embodiments provided herein, a transmembrane region and an intracellular signaling region.

Also provided herein is an anti-ROR1 chimeric antigen receptor (CAR) containing: an extracellular antigen-binding domain containing a heavy chain variable (V_{H}) region, and a light chain variable (V_{L}) region, and an intracellular signaling region, wherein: (i) the V_{H} region contains a heavy chain complementarity determining region 1 (CDR-H1) containing the sequence set forth in SEQ ID NO: 67, 82 or 52, a heavy chain complementarity determining region 2 (CDR-H2) containing the sequence set forth in SEQ ID NO: 71, 86, 56 or 97, and a heavy chain complementarity determining region 3 (CDR-H3) containing the sequence set forth in SEQ ID NO: 73, 88, 58 or 99, and the V_{L} region contains a light chain complementarity determining region 1 (CDR-L1) containing the sequence set forth in SEQ ID NO: 75, 90 or 60, a light chain complementarity determining region 2 (CDR-L2) containing the sequence set forth in SEQ ID NO: 77, 92 or 62; and a light chain complementarity determining region 3 (CDR-L3) containing the sequence set forth in SEQ ID NO: 79, 94 or 64; or (ii) the V_{H} region contains a CDR-H1 containing the sequence set forth in SEQ ID NO: 65, 80 or 50, a CDR-H2 containing the sequence set forth in SEQ ID NO: 69, 84, 54 or 95, and a CDR-H3 containing the sequence set forth in SEQ ID NO: 73, 88, 58 or 99, and the V_{L} region contains a CDR-L1 containing the sequence set forth in SEQ ID NO: 75, 90 or 60, a CDR-L2 containing the sequence set forth in SEQ ID NO: 77, 92 or 62; and a CDR-L3 containing the sequence set forth in SEQ ID NO: 79, 94 or 64; or (iii) the V_{H} region contains a CDR-H1 containing the sequence set forth in SEQ ID NO: 66, 81 or 51, a CDR-H2 containing the sequence set forth in SEQ ID NO: 70, 85, 55 or 96, and a CDR-H3 containing the sequence set forth in SEQ ID NO: 73, 88, 58 or 99, and the V_{L} region contains a CDR-L1 containing the sequence set forth in SEQ ID NO: 75, 90 or 60, a CDR-L2 containing the sequence set forth in SEQ ID NO: 77, 92 or 62; and a CDR-L3 containing the sequence set forth in SEQ ID NO: 79, 94 or 64; or (iv) the V_{H} region contains a CDR-H1 containing the sequence set forth in SEQ ID NO: 68, 83 or 53, a CDR-H2 containing the sequence set forth in SEQ ID NO: 72, 87, 57 or 98, and a CDR-H3 containing the sequence set forth in SEQ ID NO: 74, 89, 59 or 100, and the V_{L} region contains a CDR-L1 containing the sequence set forth in SEQ ID NO: 76, 91 or 61, a CDR-L2 containing the sequence set forth in SEQ ID NO: 78, 93 or 63; and a CDR-L3 containing the sequence set forth in SEQ ID NO: 79, 94 or 64.

Also provided herein is an anti-ROR1 chimeric antigen receptor (CAR) containing: an extracellular antigen-binding domain containing a heavy chain variable (V_{H}) region, and a light chain variable (V_{L}) region, and an intracellular signaling region, wherein: (i) the V_{H} region contains a heavy chain complementarity determining region 1 (CDR-H1), a heavy chain complementarity determining region 2 (CDR-H2) and a heavy chain complementarity determining region 3 (CDR-H3) containing the sequence set forth in SEQ ID NOS:67, 71 and 73, respectively, and the V_{L} region contains a light chain complementarity determining region 1 (CDR-L1), a light chain complementarity determining region 2 (CDR-L2) and a light chain complementarity determining region 3 (CDR-L3) containing the sequence set forth in SEQ ID NOS:75, 77 and 79, respectively; the V_{H} region contains a CDR-H1, a CDR-H2 and a CDR-H3 containing the sequence set forth in SEQ ID NOS:82, 86 and 88, respectively, and the V_{L} region contains a CDR-L1, a CDR-L2 and a CDR-L3 containing the sequence set forth in SEQ ID NOS:90, 92 and 94, respectively; the V_{H} region contains a CDR-H1, a CDR-H2 and a CDR-H3 containing the sequence set forth in SEQ ID NOS:52, 56 and 58, respectively, and the V_{L} region contains a CDR-L1, a CDR-L2 and a CDR-L3 containing the sequence set forth in SEQ ID NOS:60, 62 and 64, respectively; or the V_{H} region contains a CDR-H1, a CDR-H2 and a CDR-H3 containing the sequence set forth in SEQ ID NOS:52, 97 and 99, respectively, and the V_{L} region contains a CDR-L1, a CDR-L2 and a CDR-L3 containing the sequence set forth in SEQ ID NOS:60, 62 and 64, respectively; (ii) the V_{H} region contains a CDR-H1, a CDR-H2 and a CDR-H3 containing the sequence set forth in SEQ ID NOS:65, 69 and 73, respectively, and the V_{L} region contains a CDR-L1, a CDR-L2 and a CDR-L3 containing the sequence set forth in SEQ ID NOS:75, 77 and 79, respectively; the V_{H} region contains a CDR-H1, a CDR-H2 and a CDR-H3 containing the sequence set forth in SEQ ID NOS:80, 84 and 88, respectively, and the V_{L} region contains a CDR-L1, a CDR-L2 and a CDR-L3 containing the sequence set forth in SEQ ID NOS:90, 92 and 94, respectively; the V_{H} region contains a CDR-H1, a CDR-H2 and a CDR-H3 containing the sequence set forth in SEQ ID NOS:50, 54 and 58, respectively, and the V_{L} region contains a CDR-L1, a CDR-L2 and a CDR-L3 containing the sequence set forth in SEQ ID NOS:60, 62 and 64, respectively; or the V_{H} region contains a CDR-H1, a CDR-H2 and a CDR-H3 containing the sequence set forth in SEQ ID NOS:50, 95 and 99, respectively, and the V_{L} region contains a CDR-L1, a CDR-L2 and a CDR-L3 containing the sequence set forth in SEQ ID NOS:60, 62 and 64, respectively; (iii) the V_{H} region contains a CDR-H1, a CDR-H2 and a CDR-H3 containing the sequence set forth in SEQ ID NOS:66, 70 and 73, respectively, and the V_{L} region contains a CDR-L1, a CDR-L2 and a CDR-L3 containing the sequence set forth in SEQ ID NOS:75, 77 and 79, respectively; the V_{H} region contains a CDR-H1, a CDR-H2 and a CDR-H3 containing the sequence set forth in SEQ ID NOS:81, 85 and 88, respectively, and the V_{L} region contains a CDR-L1, a CDR-L2 and a CDR-L3 containing the sequence set forth in SEQ ID NOS:90, 92 and 94, respectively; the V_{H} region contains a CDR-H1, a CDR-H2 and a CDR-H3 containing the sequence set forth in SEQ ID NOS:51, 55 and 58, respectively, and the V_{L} region contains a CDR-L1, a CDR-L2 and a CDR-L3 containing the sequence set forth in SEQ ID NOS:60, 62 and 64, respectively; or the V_{H} region contains a CDR-H1, a CDR-H2 and a CDR-H3 containing the sequence set forth in SEQ ID NOS:51, 96 and 99, respectively, and the V_{L} region contains a CDR-L1, a CDR-L2 and a CDR-L3 containing the sequence set forth in SEQ ID NOS:60, 62 and 64, respectively; (iv) the V_{H} region contains a CDR-H1, a CDR-H2 and a CDR-H3 containing the sequence set forth in SEQ ID NOS:68, 72 and 74, respectively, and the V_{L} region contains a CDR-L1, a CDR-L2 and a CDR-L3 containing the sequence set forth in SEQ ID NOS:76, 78 and 79, respectively; or the V_{H} region contains a CDR-H1, a CDR-H2 and a CDR-H3 containing the sequence set forth in SEQ ID NOS:83, 87 and 89, respectively, and the V_{L} region contains a CDR-L1, a CDR-L2 and a CDR-L3 containing the sequence set forth in SEQ ID NOS:91, 93 and 94, respectively; the V_{H} region contains a CDR-H1, a CDR-H2 and a CDR-H3 containing the sequence set forth in SEQ ID NOS:53, 57 and 59, respectively, and the V_{L} region contains a CDR-L1, a CDR-L2 and a CDR-L3 containing the sequence set forth in SEQ ID NOS:60, 63 and 64, respectively; or the V_{H} region contains a CDR-H1, a CDR-H2 and a CDR-H3 containing the sequence set forth in SEQ ID NOS:53, 98 and 100, respectively, and the V_{L} region contains a CDR-L1, a CDR-L2 and a CDR-L3 containing the sequence set forth in SEQ ID NOS:61, 63 and 64, respectively.

In some of any such embodiments: (i) the V_{H} region contains a CDR-H1, a CDR-H2 and a CDR-H3 containing the sequence set forth in SEQ ID NOS:67, 71 and 73, respectively, and the V_{L} region contains a CDR-L1, a CDR-L2 and a CDR-L3 containing the sequence set forth in SEQ ID NOS:75, 77 and 79, respectively; (ii) the V_{H} region contains a CDR-H1, a CDR-H2 and a CDR-H3 containing the sequence set forth in SEQ ID NOS:65, 69 and 73, respectively, and the V_{L} region contains a CDR-L1, a CDR-L2 and a CDR-L3 containing the sequence set forth in SEQ ID NOS:75, 77 and 79, respectively; (iii) the V_{H} region contains a CDR-H1, a CDR-H2 and a CDR-H3 containing the sequence set forth in SEQ ID NOS:66, 70 and 73, respectively, and the V_{L} region contains a CDR-L1, a CDR-L2 and a CDR-L3 containing the sequence set forth in SEQ ID NOS:75, 77 and 79, respectively; or (iv) the V_{H} region contains a CDR-H1, a CDR-H2 and a CDR-H3 containing the sequence set forth in SEQ ID NOS:68, 72 and 74, respectively, and the V_{L} region contains a CDR-L1, a CDR-L2 and a CDR-L3 containing the sequence set forth in SEQ ID NOS:76, 78 and 79, respectively.

In some of any such embodiments: (i) the V_{H} region contains a CDR-H1, a CDR-H2 and a CDR-H3 containing the sequence set forth in SEQ ID NOS:82, 86 and 88, respectively, and the V_{L} region contains a CDR-L1, a CDR-L2 and a CDR-L3 containing the sequence set forth in SEQ ID NOS:90, 92 and 94, respectively; (ii) the V_{H} region contains a CDR-H1, a CDR-H2 and a CDR-H3 containing the sequence set forth in SEQ ID NOS:80, 84 and 88, respectively, and the V_{L} region contains a CDR-L1, a CDR-L2 and a CDR-L3 containing the sequence set forth in SEQ ID NOS:90, 92 and 94, respectively; (iii) the V_{H} region contains a CDR-H1, a CDR-H2 and a CDR-H3 containing the sequence set forth in SEQ ID NOS:81, 85 and 88, respectively, and the V_{L} region contains a CDR-L1, a CDR-L2 and a CDR-L3 containing the sequence set forth in SEQ ID NOS:90, 92 and 94, respectively; or (iv) the V_{H} region contains a CDR-H1, a CDR-H2 and a CDR-H3 containing the sequence set forth in SEQ ID NOS:83, 87 and 89, respectively, and the V_{L} region contains a CDR-L1, a CDR-L2 and a CDR-L3 containing the sequence set forth in SEQ ID NOS:91, 93 and 94, respectively.

Also provided herein is an anti-ROR1 chimeric antigen receptor (CAR) containing: an extracellular antigen-binding domain containing a heavy chain variable (V_{H}) region and a light chain variable (V_{L}) region, and an intracellular signaling region, wherein: the V_{H} region contains a heavy chain complementarity determining region 1 (CDR-H1), a heavy chain complementarity determining region 2 (CDR-H2) and a heavy chain complementarity determining region 3 (CDR-H3) contained within SEQ ID NO: 112, 121, 103 or 130, and the V_{L} region contains a light chain complementarity determining region 1 (CDR-L1), a light chain complementarity determining region 2 (CDR-L2) and a light chain complementarity determining region 3 (CDR-L3) contained within SEQ ID NO: 115, 124 or 106.

Also provided herein is an anti-ROR1 chimeric antigen receptor (CAR) containing: an extracellular antigen-binding domain containing a heavy chain variable (V_{H}) region and a light chain variable (V_{L}) region, and an intracellular signaling region, wherein: the V_{H} region contains a heavy chain complementarity determining region 1 (CDR-H1), a heavy chain complementarity determining region 2 (CDR-H2) and a heavy chain complementarity determining region 3 (CDR-H3) contained within SEQ ID NO:112, and the V_{L} region contains a light chain complementarity determining region 1 (CDR-L1), a light chain complementarity determining region 2 (CDR-L2) and a light chain complementarity determining region 3 (CDR-L3) contained within SEQ ID NO:115; the V_{H} region contains a CDR-H1, a CDR-H2 and a CDR-H3 contained within SEQ ID NO:121, and the V_{L} region contains a CDR-L1, a CDR-L2 and a CDR-L3 contained within SEQ ID: NO 124; the V_{H} region contains a CDR-H1, a CDR-H2 and a CDR-H3 contained within SEQ ID NO:103, and the V_{L} region contains a CDR-L1, a CDR-L2 and a CDR-L3 contained within SEQ ID: NO 106; or the V_{H} region contains a CDR-H1, a CDR-H2 and a CDR-H3 contained within SEQ ID NO:130, and the V_{L} region contains a CDR-L1, a CDR-L2 and a CDR-L3 contained within SEQ ID: NO 106.

In some of any such embodiments, the V_{H} region contains a CDR-H1, a CDR-H2 and a CDR-H3 contained within SEQ ID NO:112, and the V_{L} region contains a CDR-L1, a CDR-L2 and a CDR-L3 contained within SEQ ID: NO 115. In some of any such embodiments, the V_{H} region contains a CDR-H1, a CDR-H2 and a CDR-H3 contained within SEQ ID NO:121, and the V_{L} region contains a CDR-L1, a CDR-L2 and a CDR-L3 contained within SEQ ID: NO 124.

In some of any such embodiments: the V_{H} region is or contains an amino acid sequence having at least at or about 85%, 86%, 87%, 88%, 89%, 90%, 91%, 92%, 93%, 94%, 95%, 96%, 97%, 98% or 99% identity to SEQ ID NO: 112, 121, 103 or 130, and the V_{L} region is or contains an amino acid sequence having at least at or about 85%, 86%, 87%, 88%, 89%, 90%, 91%, 92%, 93%, 94%, 95%, 96%, 97%, 98% or 99% identity to SEQ ID NO: 115, 124 or 106. In some of any such embodiments: the V_{H} region is or contains an amino acid sequence having at least at or about 85%, 86%, 87%, 88%, 89%, 90%, 91%, 92%, 93%, 94%, 95%, 96%, 97%, 98% or 99% identity to SEQ ID NO:112, and the V_{L} region is or contains an amino acid sequence having at least at or about 85%, 86%, 87%, 88%, 89%, 90%, 91%, 92%, 93%, 94%, 95%, 96%, 97%, 98% or 99% identity to SEQ ID NO: 115; the V_{H} region is or contains an amino acid sequence having at least at or about 85%, 86%, 87%, 88%, 89%, 90%, 91%, 92%, 93%, 94%, 95%, 96%, 97%, 98% or 99% identity to SEQ ID NO:121, and the V_{L} region is or contains an amino acid sequence having at least at or about 85%, 86%, 87%, 88%, 89%, 90%, 91%, 92%, 93%, 94%, 95%, 96%, 97%, 98% or 99% identity to SEQ ID NO:124; the V_{H} region is or contains an amino acid sequence having at least at or about 85%, 86%, 87%, 88%, 89%, 90%, 91%, 92%, 93%, 94%, 95%, 96%, 97%, 98% or 99% identity to SEQ ID NO:103, and the V_{L} region is or contains an amino acid sequence having at least at or about 85%, 86%, 87%, 88%, 89%, 90%, 91%, 92%, 93%, 94%, 95%, 96%, 97%, 98% or 99% identity to SEQ ID NO:106; or the V_{H} region is or contains an amino acid sequence having at least at or about 85%, 86%, 87%, 88%, 89%, 90%, 91%, 92%, 93%, 94%, 95%, 96%, 97%, 98% or 99% identity to SEQ ID NO:130, and the V_{L} region is or contains an amino acid sequence having at least at or about 85%, 86%, 87%, 88%, 89%, 90%, 91%, 92%, 93%, 94%, 95%, 96%, 97%, 98% or 99% identity to SEQ ID NO:106.

In some of any such embodiments, the V_{H} region is or contains an amino acid sequence having at least at or about 85%, 86%, 87%, 88%, 89%, 90%, 91%, 92%, 93%, 94%, 95%, 96%, 97%, 98% or 99% identity to SEQ ID NO:112, and the V_{L} region is or contains an amino acid sequence having at least at or about 85%, 86%, 87%, 88%, 89%, 90%, 91%, 92%, 93%, 94%, 95%, 96%, 97%, 98% or 99% identity to SEQ ID NO: 115. In some of any such embodiments, the V_{H} region is or contains an amino acid sequence having at least at or about 85%, 86%, 87%, 88%, 89%, 90%, 91%, 92%, 93%, 94%, 95%, 96%, 97%, 98% or 99% identity to SEQ ID NO:121, and the V_{L} region is or contains an amino acid sequence having at least at or about 85%, 86%, 87%, 88%, 89%, 90%, 91%, 92%, 93%, 94%, 95%, 96%, 97%, 98% or 99% identity to SEQ ID NO:124.

Also provided herein is an anti-ROR1 chimeric antigen receptor (CAR) containing: an extracellular antigen-binding domain containing a heavy chain variable (V_{H}) region and a light chain variable (V_{L}) region, and an intracellular signaling region, wherein: the V_{H} region is or contains an amino acid sequence having at least at or about 85%, 86%, 87%, 88%, 89%, 90%, 91%, 92%, 93%, 94%, 95%, 96%, 97%, 98% or 99% identity to SEQ ID NO: 112, 121, 103 or 130, and the V_{L} region is or contains an amino acid sequence having at least at or about 85%, 86%, 87%, 88%, 89%, 90%, 91%, 92%, 93%, 94%, 95%, 96%, 97%, 98% or 99% identity to SEQ ID NO: 115, 124 or 106.

Also provided herein is an anti-ROR1 chimeric antigen receptor (CAR) containing: an extracellular antigen-binding domain containing a heavy chain variable (V_{H}) region and a light chain variable (V_{L}) region, and an intracellular signaling region, wherein: the V_{H} region is or contains an amino acid sequence having at least at or about 85%, 86%, 87%, 88%, 89%, 90%, 91%, 92%, 93%, 94%, 95%, 96%, 97%, 98% or 99% identity to SEQ ID NO:112, and the V_{L} region is or contains an amino acid sequence having at least at or about 85%, 86%, 87%, 88%, 89%, 90%, 91%, 92%, 93%, 94%, 95%, 96%, 97%, 98% or 99% identity to SEQ ID NO: 115; the V_{H} region is or contains an amino acid sequence having at least at or about 85%, 86%, 87%, 88%, 89%, 90%, 91%, 92%, 93%, 94%, 95%, 96%, 97%, 98% or 99% identity to SEQ ID NO:121, and the V_{L} region is or contains an amino acid sequence having at least at or about 85%, 86%, 87%, 88%, 89%, 90%, 91%, 92%, 93%, 94%, 95%, 96%, 97%, 98% or 99% identity to SEQ ID NO:124; the V_{H} region is or contains an amino acid sequence having at least at or about 85%, 86%, 87%, 88%, 89%, 90%, 91%, 92%, 93%, 94%, 95%, 96%, 97%, 98% or 99% identity to SEQ ID NO:103, and the V_{L} region is or contains an amino acid sequence having at least at or about 85%, 86%, 87%, 88%, 89%, 90%, 91%, 92%, 93%, 94%, 95%, 96%, 97%, 98% or 99% identity to SEQ ID NO:106; or the V_{H} region is or contains an amino acid sequence having at least at or about 85%, 86%, 87%, 88%, 89%, 90%, 91%, 92%, 93%, 94%, 95%, 96%, 97%, 98% or 99% identity to SEQ ID NO:130, and the V_{L} region is or contains an amino acid sequence having at least at or about 85%, 86%, 87%, 88%, 89%, 90%, 91%, 92%, 93%, 94%, 95%, 96%, 97%, 98% or 99% identity to SEQ ID NO:106.

In some of any such embodiments, the V_{H} region is or contains an amino acid sequence having at least at or about 85%, 86%, 87%, 88%, 89%, 90%, 91%, 92%, 93%, 94%, 95%, 96%, 97%, 98% or 99% identity to SEQ ID NO:112, and the V_{L} region is or contains an amino acid sequence having at least at or about 85%, 86%, 87%, 88%, 89%, 90%, 91%, 92%, 93%, 94%, 95%, 96%, 97%, 98% or 99% identity to SEQ ID NO: 115. In some of any such embodiments, the V_{H} region is or contains an amino acid sequence having at least at or about 85%, 86%, 87%, 88%, 89%, 90%, 91%, 92%, 93%, 94%, 95%, 96%, 97%, 98% or 99% identity to SEQ ID NO:121, and the V_{L} region is or contains an amino acid sequence having at least at or about 85%, 86%, 87%, 88%, 89%, 90%, 91%, 92%, 93%, 94%, 95%, 96%, 97%, 98% or 99% identity to SEQ ID NO:124.

In some of any such embodiments: the V_{H} region is or contains the sequence set forth in SEQ ID NO: 112, 121, 103 or 130, and the V_{L} region is or contains the sequence set forth in SEQ ID NO: 115, 124 or 106.

In some of any such embodiments: the V_{H} region and the V_{L} region are or contain the sequence set forth in SEQ ID NOS: 112 and 115, respectively; the V_{H} region and the V_{L} region are or contain the sequence set forth in SEQ ID NOS: 121 and 124, respectively; the V_{H} region and the V_{L} region are or contain the sequence set forth in SEQ ID NOS: 103 and 106, respectively; or the V_{H} region and the V_{L} region are or contain the sequence set forth in SEQ ID NOS: 130 and 106, respectively.

Also provided herein is an anti-ROR1 chimeric antigen receptor (CAR) containing: an extracellular antigen-binding domain containing a heavy chain variable (V_{H}) region and a light chain variable (V_{L}) region, and an intracellular signaling region, wherein: the V_{H} region is or contains the sequence set forth in SEQ ID NO: 112, 121, 103 or 130, and the V_{L} region is or contains the sequence set forth in SEQ ID NO: 115, 124 or 106.

Also provided herein is an anti-ROR1 chimeric antigen receptor (CAR) containing: an extracellular antigen-binding domain containing a heavy chain variable (V_{H}) region and a light chain variable (V_{L}) region, and an intracellular signaling region, wherein: the V_{H} region and the V_{L} region are or contain the sequence set forth in SEQ ID NOS: 112 and 115, respectively; the V_{H} region and the V_{L} region are or contain the sequence set forth in SEQ ID NOS: 121 and 124, respectively; the V_{H} region and the V_{L} region are or contain the sequence set forth in SEQ ID NOS: 103 and 106, respectively; or the V_{H} region and the V_{L} region are or contain the sequence set forth in SEQ ID NOS: 130 and 106, respectively.

In some of any such embodiments, the V_{H} region and the V_{L} region are or contain the sequence set forth in SEQ ID NOS: 112 and 115, respectively. In some of any such embodiments, the V_{H} region and the V_{L} region are or contain the sequence set forth in SEQ ID NOS:121 and 124, respectively.

In some of any such embodiments, the V_{H} region is or contains the amino acid sequence encoded by SEQ ID NO: 110, 119, 101 or 128 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 110, 119, 101 or 128, and the V_{L} region is or contains the amino acid sequence encoded by SEQ ID NO: 113, 122 or 104, or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 113, 122 or 104. In some of any such embodiments, the V_{H} region is or contains the amino acid sequence encoded by SEQ ID NO: 111, 120, 102 or 129, and the V_{L} region is or contains the amino acid sequence encoded by SEQ ID NO: 114, 123, 105 or 131. In some of any such embodiments, the V_{H} region is or contains the amino acid sequence encoded by SEQ ID NO: 110 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 110, and the V_{L} region is or contains the amino acid sequence encoded by SEQ ID NO: 113, or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 113. In some of any such embodiments, the V_{H} region is or contains the amino acid sequence encoded by SEQ ID NO: 111, and the V_{L} region is or contains the amino acid sequence encoded by SEQ ID NO: 114. In some of any such embodiments, the V_{H} region is or contains the amino acid sequence encoded by SEQ ID NO: 119 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 119, and the V_{L} region is or contains the amino acid sequence encoded by SEQ ID NO: 122, or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 122. In some of any such embodiments, the V_{H} region is or contains the amino acid sequence encoded by SEQ ID NO: 120, and the V_{L} region is or contains the amino acid sequence encoded by SEQ ID NO: 123.

In some of any such embodiments, said anti-ROR1 antibody or antigen-binding fragment thereof is isolated. In some of any such embodiments, said anti-ROR1 antibody or antigen-binding fragment thereof is recombinant. In some of any such embodiments, at least a portion of the V_{H} region and the V_{L} region is human or is from a human protein.

In some of any such embodiments, the antigen-binding fragment thereof is or contains a single chain fragment. In some of any such embodiments, the antigen-binding fragment thereof is or contains a single chain Fv (scFv).

In some of any such embodiments, the V_{H} region is amino-terminal to the V_{L} region. In some of any such embodiments, the V_{H} region is carboxy-terminal to the V_{L} region. In some of any such embodiments, the V_{H} region and the V_{L} region are joined by a flexible linker. In some of any such embodiments, the flexible linker contains the sequence set forth in SEQ ID NO:41.

In some of any such embodiments, the scFv is or contains the sequence set forth in SEQ ID NO: 118, 127, 109 or 134, or an amino acid sequence having at least at or about 85%, 86%, 87%, 88%, 89%, 90%, 91%, 92%, 93%, 94%, 95%, 96%, 97%, 98% or 99% sequence identity to SEQ ID NO: 118, 127, 109 or 134. In some of any such embodiments, the scFv is or contains the sequence set forth in SEQ ID NO: 118. In some of any such embodiments, the scFv is or contains the sequence set forth in SEQ ID NO: 127. In some of any such embodiments, the scFv is or contains the amino acid sequence encoded by SEQ ID NO: 116, 125, 107 or 132 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 116, 125, 107 or 132. In some of any such embodiments, the scFv is or contains the amino acid sequence encoded by SEQ ID NO: 117, 126, 108 or 133. In some of any such embodiments, the scFv is or contains the amino acid sequence encoded by SEQ ID NO: 116 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 116. In some of any such embodiments, the scFv is or contains the amino acid sequence encoded by SEQ ID NO: 117. In some of any such embodiments, the scFv is or contains the amino acid sequence encoded by SEQ ID NO: 125 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 125. In some of any such embodiments, the scFv is or contains the amino acid sequence encoded by SEQ ID NO: 126.

In some of any such embodiments, the anti-ROR1 chimeric antigen receptor further contains a spacer between the extracellular antigen-binding domain and the transmembrane domain. In some of any such embodiments, the spacer contains at least a portion of an immunoglobulin or a variant thereof.

In some of any such embodiments, the spacer contains at least a portion of a hinge region of an immunoglobulin or a variant thereof. In some of any such embodiments, the spacer is less than at or about 15 amino acids in length. In some of any such embodiments, the spacer is or contains an amino acid sequence having at least at or about 85%, 86%, 87%, 88%, 89%, 90%, 91%, 92%, 93%, 94%, 95%, 96%, 97%, 98% or 99% identity to SEQ ID NO:1, 26, 27, 29, 31, 32, 33 or 135.

In some of any such embodiments, the at least a portion of a hinge region contains all or a portion of an IgG4 hinge region. In some of any such embodiments, the at least a portion of a hinge region contains all or a portion of a human IgG4 hinge region, or a variant thereof. In some of any such embodiments, the at least a portion of a hinge region contains all or a portion of an IgG2 hinge region. In some of any such embodiments, the at least a portion of a hinge region contains all or a portion of a human IgG2 hinge region, or a variant thereof.

In some of any such embodiments, the spacer is or contains the sequence set forth in SEQ ID NO:1. In some of any such embodiments, the spacer is or contains the amino acid sequence encoded by SEQ ID NO: 2 or 30 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 2 or 30. In some of any such embodiments, the spacer is or contains the amino acid sequence encoded by SEQ ID NO: 30.

In some of any such embodiments, the spacer is or contains the sequence set forth in SEQ ID NO:135. In some of any such embodiments, the spacer is or contains the amino acid sequence encoded by SEQ ID NO: 192 or 136 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 192 or 136. In some of any such embodiments, the spacer is or contains the amino acid sequence encoded by SEQ ID NO: 136.

In some of any such embodiments, the spacer contains at least a portion of a C_{H}3 region of an immunoglobulin or a variant thereof. In some of any such embodiments, the at least a portion of a C_{H}3 region contains all or a portion of an IgG4 C_{H}3. In some of any such embodiments, the at least a portion of a C_{H}3 region contains all or a portion of an IgG2 C_{H}3. In some of any such embodiments, the at least a portion of a C_{H}3 region contains all or a portion of an IgG4 C_{H}3 and/or an IgG2 C_{H}3. In some of any such embodiments, the IgG4 C_{H}3 is a human IgG4 C_{H}3 and the IgG2 C_{H}3 is a human IgG2 C_{H}3. In some of any such embodiments, the spacer contains at least a portion of a hinge region and at least a portion of a C_{H}3 region of an immunoglobulin or a variant thereof. In some of any such embodiments, the spacer is at or about 111, 112, 113, 114, 115, 116, 117, 118, 119, 120, 121, 122, 123, 124 or 125 amino acids in length, or has a length between any of the foregoing. In some of any such embodiments, the spacer is at or about 120 amino acids in length.

In some of any such embodiments, the spacer is or contains an amino acid sequence having at least at or about 85%, 86%, 87%, 88%, 89%, 90%, 91%, 92%, 93%, 94%, 95%, 96%, 97%, 98% or 99% identity to SEQ ID NO: 138. In some of any such embodiments, the spacer is or contains the sequence set forth in SEQ ID NO:138. In some of any such embodiments, the spacer is or contains the amino acid sequence encoded by SEQ ID NO: 193 or 139 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 193 or 139. In some of any such embodiments, the spacer is or contains the amino acid sequence encoded by SEQ ID NO: 139.

In some of any such embodiments, the spacer is at or about 120 amino acids in length. In some of any such embodiments, the spacer is or contains an amino acid sequence having at least at or about 85%, 86%, 87%, 88%, 89%, 90%, 91%, 92%, 93%, 94%, 95%, 96%, 97%, 98% or 99% identity to SEQ ID NO: 3. In some of any such embodiments, the spacer is or contains the sequence set forth in SEQ ID NO:3. In some of any such embodiments, the spacer is or contains the amino acid sequence encoded by SEQ ID NO: 4 or 137 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 4 or 137. In some of any such embodiments, the spacer is or contains the amino acid sequence encoded by SEQ ID NO: 137.

In some of any such embodiments, the spacer contains at least a portion of a C_{H}2 of an immunoglobulin or a variant thereof. In some of any such embodiments, the at least a portion of a C_{H}2 region contains all or a portion of an IgG4 C_{H}2. In some of any such embodiments, the at least a portion of a C_{H}2 region contains all or a portion of an IgG2 C_{H}2. In some of any such embodiments, the at least a portion of a C_{H}2 region contains all or a portion of an IgG4 C_{H}2 and/or an IgG2 C_{H}2. In some of any such embodiments, the IgG4 C_{H}2 is a human IgG4 C_{H}2 and the IgG2 C_{H}2 is a human IgG2 C_{H}2. In some of any such embodiments, the spacer contains at least a portion of a hinge region, at least a portion of a C_{H}2 and at least a portion of a C_{H}3 region of an immunoglobulin or a variant thereof. In some of any such embodiments, the spacer is at or about 130, 140, 150, 160, 170, 180, 190, 200, 210, 220, 221, 222, 223, 224, 225, 226, 227, 228, 229 or 230 amino acids in length, or has a length between any of the foregoing. In some of any such embodiments, one or more of the hinge region, the C_{H}2 region and the C_{H}3 region contains all or a portion of a C_{H}2 region and all or a portion of a C_{H}3 region from human IgG4. In some of any such embodiments, one or more of the hinge region, the C_{H}2 region and the C_{H}3 region is chimeric and contains a hinge, a C_{H}2 region and a C_{H}3 region from human IgG4 and human IgG2.

In some of any such embodiments, the spacer contains a IgG4/2 chimeric hinge region or a modified IgG4 hinge region containing at least one amino acid replacement compared to a human IgG4 hinge; an IgG2/4 chimeric C_{H}2 region; and an IgG4 C_{H}3 region.

In some of any such embodiments, the spacer is or contains an amino acid sequence having at least at or about 85%, 86%, 87%, 88%, 89%, 90%, 91%, 92%, 93%, 94%, 95%, 96%, 97%, 98% or 99% identity to SEQ ID NO:194. In some of any such embodiments, the spacer is or contains the sequence set forth in SEQ ID NO: 194. In some of any such embodiments, the spacer is or contains the amino acid sequence encoded by SEQ ID NO: 195 or 196 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 195 or 196. In some of any such embodiments, the spacer is or contains the amino acid sequence encoded by SEQ ID NO: 196.

In some of any such embodiments, the spacer is or contains an amino acid sequence having at least at or about 85%, 86%, 87%, 88%, 89%, 90%, 91%, 92%, 93%, 94%, 95%, 96%, 97%, 98% or 99% identity to SEQ ID NO:37. In some of any such embodiments, the spacer is or contains the sequence set forth in SEQ ID NO: 37. In some of any such embodiments, the spacer is or contains the amino acid sequence encoded by SEQ ID NO: 38 or 140 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 38 or 140. In some of any such embodiments, the spacer is or contains the amino acid sequence encoded by SEQ ID NO: 140.

In some of any such embodiments, the intracellular signaling region contains an intracellular signaling domain. In some of any such embodiments, the intracellular signaling domain is capable of inducing a primary activation signal in a T cell, is a T cell receptor (TCR) component or contains an immunoreceptor tyrosine-based activation motif (ITAM). In some of any such embodiments, the intracellular signaling domain is or contains a cytoplasmic signaling domain of a CD3-zeta (CD3ζ chain or a functional variant or signaling portion thereof. In some of any such embodiments, the intracellular signaling domain is human or is from a human protein. In some of any such embodiments, the intracellular signaling domain is or contains the sequence set forth in SEQ ID NO:13, 14 or 15, or an amino acid sequence having at least at or about 90%, 91%, 92%, 93%, 94%, 95%, 96%, 97%, 98% or 99% sequence identity to SEQ ID NO:13, 14 or 15. In some of any such embodiments, the intracellular signaling domain is or contains the sequence set forth in SEQ ID NO:13.

In some of any such embodiments, the intracellular signaling region further contains a costimulatory signaling region. In some of any such embodiments, the costimulatory signaling region contains an intracellular signaling domain of a T cell costimulatory molecule or a signaling portion thereof. In some of any such embodiments, the costimulatory signaling region contains an intracellular signaling domain of CD28, 4-1BB, or ICOS, or a signaling portion thereof. In some of any such embodiments, the costimulatory signaling region is human or is from a human protein. In some of any such embodiments, the costimulatory signaling region contains an intracellular signaling domain of CD28. In some of any such embodiments, the costimulatory signaling region is or contains the sequence set forth in SEQ ID NO:10 or an amino acid sequence having at least at or about 90%, 91%, 92%, 93%, 94%, 95%, 96%, 97%, 98% or 99% sequence identity to SEQ ID NO:10. In some of any such embodiments, the costimulatory signaling region contains an intracellular signaling domain of 4-1BB. In some of any such embodiments, the costimulatory signaling region is or contains the sequence set forth in SEQ ID NO:12 or an amino acid sequence having at least at or about 90%, 91%, 92%, 93%, 94%, 95%, 96%, 97%, 98% or 99% sequence identity to SEQ ID NO: 12.

In some of any such embodiments, the anti-ROR1 chimeric antigen receptor further contains a transmembrane region. In some of any such embodiments, the costimulatory signaling region is between the transmembrane region and the intracellular signaling domain. In some of any such embodiments, the transmembrane region is or contains a transmembrane domain from CD4, CD28, or CD8. In some of any such embodiments, the transmembrane region is or contains a transmembrane domain from CD28. In some of any such embodiments, the transmembrane region is human or is from a human protein.

In some of any such embodiments, the transmembrane domain is or contains SEQ ID NO: 8 or an amino acid sequence having at least at or about 90%, 91%, 92%, 93%, 94%, 95%, 96%, 97%, 98% or 99% sequence identity to SEQ ID NO: 8. In some of any such embodiments, the transmembrane domain is or contains the sequence set forth in SEQ ID NO: 8. In some of any such embodiments, the transmembrane domain is or contains the amino acid sequence encoded by SEQ ID NO: 197 or 198 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 197 or 198. In some of any such embodiments, the transmembrane domain is or contains the amino acid sequence encoded by SEQ ID NO: 198.

In some of any such embodiments, the transmembrane domain is or contains SEQ ID NO: 149 or an amino acid sequence having at least at or about 90%, 91%, 92%, 93%, 94%, 95%, 96%, 97%, 98% or 99% sequence identity to SEQ ID NO: 149. In some of any such embodiments, the transmembrane domain is or contains the sequence set forth in SEQ ID NO: 149. In some of any such embodiments, the transmembrane domain is or contains the amino acid sequence encoded by SEQ ID NO: 147 or 148 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 147 or 148. In some of any such embodiments, the transmembrane domain is or contains the amino acid sequence encoded by SEQ ID NO: 148.

In some of any such embodiments, the encoded chimeric antigen receptor contains from its N to C terminus in order: the extracellular antigen-binding domain, the spacer, the transmembrane region and the intracellular signaling region. In some of any such embodiments, the encoded chimeric antigen receptor contains, from its N to C terminus in order: an extracellular antigen-binding domain containing an scFv, a spacer containing a modified IgG4 hinge; a transmembrane domain; and an intracellular signaling region containing a cytoplasmic signaling domain of a CD3-zeta (CD3ζ chain and an intracellular signaling domain of a costimulatory signaling region. In some of any such embodiments, the encoded chimeric antigen receptor contains, from its N to C terminus in order: an extracellular antigen-binding domain containing an scFv, a spacer containing a modified IgG4 hinge containing the sequence set forth in SEQ ID NO:135; a transmembrane domain from a human CD28; and an intracellular signaling region containing a cytoplasmic signaling domain of a CD3-zeta (CD3) chain and an intracellular signaling domain of a costimulatory signaling region containing an intracellular signaling domain of 4-1BB. In some of any such embodiments, the encoded chimeric antigen receptor contains, from its N to C terminus in order: an extracellular antigen-binding domain containing an scFv, a spacer containing a modified IgG4 hinge-C_{H}3; a transmembrane domain; and an intracellular signaling region containing a cytoplasmic signaling domain of a CD3-zeta (CD3) chain and an intracellular signaling domain of a costimulatory signaling region. In some of any such embodiments, the encoded chimeric antigen receptor contains, from its N to C terminus in order: an extracellular antigen-binding domain containing an scFv, a spacer containing a modified IgG4 hinge-C_{H}3 containing the sequence set forth in SEQ ID NO: 138; a transmembrane domain from a human CD28; and an intracellular signaling region containing a cytoplasmic signaling domain of a CD3-zeta (CD3) chain and an intracellular signaling domain of a costimulatory signaling region containing an intracellular signaling domain of 4-1BB. In some of any such embodiments, the extracellular antigen-binding domain is an scFv.

In some of any such embodiments, the anti-ROR1 chimeric antigen receptor is or contains the sequence set forth in SEQ ID NO: 184, 185, 186, 187, 188 or 189 or a sequence that exhibits at least 85%, 86%, 87%, 88%, 89%, 90%, 91%, 92%, 93%, 94%, 95%, 96%, 97%, 98%, or 99% sequence identity to the sequence set forth in SEQ ID NO: 184, 185, 186, 187, 188 or 189.

In some of any such embodiments, the anti-ROR1 chimeric antigen receptor is or contains the sequence set forth in SEQ ID NO: 184 or a sequence that exhibits at least 85%, 86%, 87%, 88%, 89%, 90%, 91%, 92%, 93%, 94%, 95%, 96%, 97%, 98%, or 99% sequence identity to the sequence set forth in SEQ ID NO: 184. In some of any such embodiments, the anti-ROR1 chimeric antigen receptor is or contains the sequence set forth in SEQ ID NO: 184. In some of any such embodiments, the anti-ROR1 chimeric antigen receptor is or contains the sequence set forth in SEQ ID NO: 185 or a sequence that exhibits at least 85%, 86%, 87%, 88%, 89%, 90%, 91%, 92%, 93%, 94%, 95%, 96%, 97%, 98%, or 99% sequence identity to the sequence set forth in SEQ ID NO:185. In some of any such embodiments, the anti-ROR1 chimeric antigen receptor is or contains the sequence set forth in SEQ ID NO: 185. In some of any embodiments, the anti-ROR1 chimeric antigen receptor is or comprises the sequence set forth in SEQ ID NO: 186 or a sequence that exhibits at least 85%, 86%, 87%, 88%, 89%, 90%, 91%, 92%, 93%, 94%, 95%, 96%, 97%, 98%, or 99% sequence identity to the sequence set forth in SEQ ID NO: 186. In some of any embodiments, the anti-ROR1 chimeric antigen receptor is or comprises the sequence set forth in SEQ ID NO: 186. In some of any embodiments, the anti-ROR1 chimeric antigen receptor is or comprises the sequence set forth in SEQ ID NO: 187 or a sequence that exhibits at least 85%, 86%, 87%, 88%, 89%, 90%, 91%, 92%, 93%, 94%, 95%, 96%, 97%, 98%, or 99% sequence identity to the sequence set forth in SEQ ID NO:187. In some of any embodiments, the anti-ROR1 chimeric antigen receptor is or comprises the sequence set forth in SEQ ID NO: 187. In some of any embodiments, the anti-ROR1 chimeric antigen receptor is or comprises the sequence set forth in SEQ ID NO: 188 or a sequence that exhibits at least 85%, 86%, 87%, 88%, 89%, 90%, 91%, 92%, 93%, 94%, 95%, 96%, 97%, 98%, or 99% sequence identity to the sequence set forth in SEQ ID NO: 188. In some of any embodiments, the anti-ROR1 chimeric antigen receptor is or comprises the sequence set forth in SEQ ID NO: 188. In some of any embodiments, the anti-ROR1 chimeric antigen receptor is or comprises the sequence set forth in SEQ ID NO: 189 or a sequence that exhibits at least 85%, 86%, 87%, 88%, 89%, 90%, 91%, 92%, 93%, 94%, 95%, 96%, 97%, 98%, or 99% sequence identity to the sequence set forth in SEQ ID NO:189. In some of any embodiments, the anti-ROR1 chimeric antigen receptor is or comprises the sequence set forth in SEQ ID NO: 189.

In some of any such embodiments, the anti-ROR1 chimeric antigen receptor is encoded by the sequence set forth in SEQ ID NO: 156, 157, 158, 159, 160 or 161 or a sequence that exhibits at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 156, 157, 158, 159, 160 or 161. In some of any such embodiments, the anti-ROR1 chimeric antigen receptor is encoded by the sequence set forth in SEQ ID NO: 156 or a sequence that exhibits at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 156. In some of any such embodiments, the anti-ROR1 chimeric antigen receptor is encoded by the sequence set forth in SEQ ID NO: 156. In some of any such embodiments, the anti-ROR1 chimeric antigen receptor is encoded by the sequence set forth in SEQ ID NO: 157 or a sequence that exhibits at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 157. In some of any such embodiments, the anti-ROR1 chimeric antigen receptor is encoded by the sequence set forth in SEQ ID NO: 157.

In some of any such embodiments, said anti-ROR1 chimeric antigen receptor specifically binds to a receptor tyrosine kinase-like orphan receptor 1 (ROR1) protein. In some of any such embodiments, said anti-ROR1 chimeric antigen receptor specifically binds to a human ROR1 protein. In some of any such embodiments, the human ROR1 protein contains an amino acid sequence set forth in SEQ ID NO: 144, 145 or 146. In some of any such embodiments, said anti-ROR1 chimeric antigen receptor does not bind to, is not cross-reactive to, or binds at a lower level or degree or affinity to a receptor tyrosine kinase-like orphan receptor 2 (ROR2) protein. In some of any such embodiments, said anti-ROR1 chimeric antigen receptor does not bind to, is not cross-reactive to, or binds at a lower level or degree or affinity to a human ROR2.

In some of any such embodiments, the extent, level or degree or affinity of binding of said anti-ROR1 chimeric antigen receptor to a human ROR2 is at least at or about 75%, 80%, 90%, 95% or 99% less than the extent, level or degree or affinity of binding to a human ROR1. In some of any such embodiments, binding is compared under the same or substantially the same conditions or assay. In some of any such embodiments, said anti-ROR1 chimeric antigen receptor exhibits the same, substantially the same or lower level or degree of signaling or activity in the presence of a ROR2 protein, compared to the level or degree of signaling or activity in the presence of a ROR1 protein. In some of any such embodiments, said anti-ROR1 chimeric antigen receptor exhibits the same, substantially the same or lower level or degree of signaling or activity in the presence of a human ROR2 protein, compared to the level or degree of signaling or activity in the presence of a human ROR1 protein. In some of any such embodiments, activity is compared under the same or substantially the same conditions or assay. In some of any such embodiments, said anti-ROR1 chimeric antigen receptor exhibits a level or degree of signaling or activity in the presence of a human ROR2 that is at least at or about 75%, 80%, 90%, 95% or 99% less than the level or degree of signaling or activity in the presence of a human ROR1. In some of any such embodiments, activity is compared under the same or substantially the same conditions or assay.

In some of any such embodiments, said anti-ROR1 chimeric antigen receptor exhibits the same, substantially the same or lower level or degree of signaling or activity in the presence of a ROR2 protein compared to a reference ROR1-specific chimeric antigen receptor. In some of any such embodiments, said anti-ROR1 chimeric antigen receptor exhibits the same, substantially the same or lower level or degree of signaling or activity in the presence of a human ROR2, compared to a reference ROR1-specific chimeric antigen receptor. In some of any embodiments, activity is compared under the same or substantially the same conditions or assay.

In some of any such embodiments, said anti-ROR1 chimeric antigen receptor exhibits the same, substantially the same or lower level or degree of signaling or activity in the presence of a ROR2 protein, compared to a reference ROR1-specific chimeric antigen receptor. In some of any such embodiments, said anti-ROR1 chimeric antigen receptor exhibits the same, substantially the same or lower level or degree of signaling or activity in the presence of a human ROR2 protein, compared to a reference ROR1-specific chimeric antigen receptor. In some of any such embodiments, activity is compared under the same or substantially the same conditions or assay. In some of any such embodiments, said anti-ROR1 chimeric antigen receptor exhibits the same, substantially the same or higher antigen-specific signaling or antigen dependent activity or signaling compared to a reference ROR1-specific chimeric antigen receptor. In some of any such embodiments, activity is compared under the same or substantially the same conditions or assay. In some of any such embodiments, said anti-ROR1 chimeric antigen receptor exhibits the same, substantially the same or lower tonic signaling or antigen independent activity or signaling compared to a reference ROR1-specific chimeric antigen receptor. In some of any such embodiments, activity is compared under the same or substantially the same conditions or assay. In some of any such embodiments, said anti-ROR1 chimeric antigen receptor exhibits a level or degree of tonic signaling or antigen independent activity or signaling that is at least at or about 75%, 80%, 90%, 95% or 99% less than the level or degree of tonic signaling or antigen independent activity of a reference ROR1-specific chimeric antigen receptor. In some of any such embodiments, activity is compared under the same or substantially the same conditions or assay.

In some of any such embodiments, the reference ROR1-specific chimeric antigen receptor contains the anti-ROR1 antibody R12 or the anti-ROR1 antibody 2A2 or an antigen-binding fragment thereof. In some of any such embodiments, the reference ROR1-specific chimeric antigen receptor contains an scFv from R12 or 2A2. In some of any such embodiments, the reference ROR1-specific chimeric antigen receptor contains the anti-ROR1 antibody R12 or an scFv from R12 or 2A2.

Also provided herein is a polynucleotide containing a nucleic acid encoding all or a portion of the anti-ROR1 antibody or antigen-binding domain thereof of any of the embodiments provided herein, the single chain cell surface protein of any of the embodiments provided herein, the conjugate of any of the embodiments provided herein, or the anti-ROR1 chimeric antigen receptor of any of the embodiments provided herein.

In some of any such embodiments, said polynucleotide contains a nucleic acid encoding the V_{H} containing the sequence set forth in SEQ ID NO: 110, 119, 101 or 128 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 110, 119, 101 or 128, and a nucleic acid encoding the V_{L} containing the sequence set forth in SEQ ID NO: 113, 122 or 104, or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 113, 122 or 104. In some of any such embodiments, said polynucleotide contains a nucleic acid encoding the V_{H} containing the sequence set forth in SEQ ID NO: 110 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 110, and a nucleic acid encoding the V_{L} containing the sequence set forth in SEQ ID NO: 113, or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 113. In some of any such embodiments, said polynucleotide contains a nucleic acid encoding the V_{H} containing the sequence set forth in SEQ ID NO: 119 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 119, and a nucleic acid encoding the V_{L} containing the sequence set forth in SEQ ID NO: 122, or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 122.

In some of any such embodiments, said polynucleotide contains a nucleic acid encoding the scFv containing the sequence set forth in SEQ ID NO: 116, 125, 107 or 132 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 116, 125, 107 or 132. In some of any such embodiments, said polynucleotide contains a nucleic acid encoding the scFv containing the sequence set forth in SEQ ID NO: 116 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 116. In some of any such embodiments, said polynucleotide contains a nucleic acid encoding the scFv containing the sequence set forth in SEQ ID NO: 125 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 125.

In some of any such embodiments, said polynucleotide contains a nucleic acid encoding the spacer containing the sequence set forth in SEQ ID NO: 192 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 192. In some of any such embodiments, said polynucleotide contains a nucleic acid encoding the spacer containing the sequence set forth in SEQ ID NO: 193 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 193. In some of any such embodiments, said polynucleotide contains a nucleic acid encoding the spacer containing the sequence set forth in SEQ ID NO: 195 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 195.

In some of any such embodiments, the nucleic acid encoding the anti-ROR1 antibody or antigen-binding domain thereof, the single chain cell surface protein, the conjugate or the anti-ROR1 chimeric antigen receptor contains at least one modified splice donor or splice acceptor site or both, said modified splice donor and/or acceptor site containing one or more nucleotide modifications corresponding to a reference splice donor site and/or reference splice acceptor site. In some of any such embodiments, the one or more nucleotide modifications contain a nucleic acid substitution. In some of any such embodiments, the reference splice donor and/or reference splice acceptor sites are canonical, non-canonical, or cryptic splice sites. In some of any embodiments, the polynucleotide is optimized by splice site elimination.

In some of any such embodiments: the reference splice donor or reference splice acceptor site(s) or both has a splice site prediction score of at least at or about 0.4, 0.5, 0.6, 0.70, 0.75, 0.80, 0.85, 0.90, 0.95, 0.99 or 1.0; or the reference splice donor or reference splice acceptor site(s) or both is/are predicted to be involved in a splice event with a probability of at least at or about 40%, 50%, 60%, 70%, 75%, 80%, 85%, 90%, 95%, 99% or 100%.

In some of any such embodiments: the reference splice donor or reference splice acceptor site(s) or both has a splice site prediction score of at least at or about 0.70, 0.75, 0.80, 0.85, 0.90, 0.95, 0.99 or 1.0; and/or the reference splice donor or reference splice acceptor site(s) or both is/are predicted to be involved in a splice event with a probability of at least at or about 70%, 75%, 80%, 85%, 90%, 95%, 99% or 100%.

In some of any such embodiments, at least one of the one or more nucleotide modifications are within 1, 2, 3, 4, 5, 6, 7, 8, 9 or 10 residues of the splice site junction of the reference splice acceptor and/or reference splice donor site. In some of any such embodiments, the one or more nucleotide modifications is silent or results in a degenerate codon or does not change the amino acid sequence of the encoded protein, or one or more or all of the foregoing.

In some of any such embodiments, upon expression of the polynucleotide in a cell, the transcribed RNA from the polynucleotide, exhibits at least at or about 70%, at or about 75%, at or about 80%, at or about 85%, at or about 90%, or at or about 95% RNA homogeneity. In some of any such embodiments, upon expression in a cell, the transcribed RNA from the polynucleotide exhibits reduced heterogeneity compared to the heterogeneity of the mRNA transcribed from a reference polynucleotide, said reference polynucleotide encoding the same amino acid sequence as the polynucleotide, wherein the reference polynucleotide differs by the presence of one or more splice donor site or one or more splice acceptor site or both, in the nucleic acid encoding the spacer or contains one or more nucleotide modifications compared to the polynucleotide. In some of any such embodiments, the RNA heterogeneity is reduced by greater than at or about 10%, 15%, 20%, 25%, 30%, 40% or 50% or more.

In some of any such embodiments, the transcribed RNA from the reference polynucleotide exhibits greater than at or about 10%, 15%, 20%, 25%, 30%, 40% or 50% or more RNA heterogeneity. In some of any such embodiments, the transcribed RNA messenger RNA (mRNA). In some of any such embodiments, the RNA homogeneity or heterogeneity or both is determined by agarose gel electrophoresis, chip-based capillary electrophoresis, analytical ultracentrifugation, field flow fractionation, or liquid chromatography.

In some of any such embodiments, the polynucleotide is codon-optimized for expression in a human cell.

In some of any such embodiments, said polynucleotide contains a nucleic acid encoding the V_{H} containing the sequence set forth in SEQ ID NO: 111, 120, 102 or 129, and a nucleic acid encoding the V_{L} containing the sequence set forth in SEQ ID NO: 114, 123, 105 or 131. In some of any such embodiments, said polynucleotide contains a nucleic acid encoding the V_{H} containing the sequence set forth in SEQ ID NO: 111, and a nucleic acid encoding the V_{L} containing the sequence set forth in SEQ ID NO: 114. In some of any such embodiments, said polynucleotide contains a nucleic acid encoding the V_{H} containing the sequence set forth in SEQ ID NO: 120, and a nucleic acid encoding the V_{L} containing the sequence set forth in SEQ ID NO: 123.

In some of any such embodiments, said polynucleotide contains a nucleic acid encoding the scFv containing the sequence set forth in SEQ ID NO: 117, 126, 108 or 133. In some of any such embodiments, said polynucleotide contains a nucleic acid encoding the scFv containing the sequence set forth in SEQ ID NO: 117. In some of any such embodiments, said polynucleotide contains a nucleic acid encoding the scFv containing the sequence set forth in SEQ ID NO: 126.

In some of any such embodiments, said polynucleotide contains a nucleic acid encoding the spacer containing the sequence set forth in SEQ ID NO:136. In some of any such embodiments, said polynucleotide contains a nucleic acid encoding the spacer containing the sequence set forth in SEQ ID NO:139. In some of any such embodiments, said polynucleotide contains a nucleic acid encoding the spacer containing the sequence set forth in SEQ ID NO:196.

In some of any such embodiments, said polynucleotide contains the sequence set forth in SEQ ID NO: 156, 157, 158, 159, 160 or 161 or a sequence that exhibits at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 156, 157, 158, 159, 160 or 161. In some of any such embodiments, said polynucleotide contains the sequence set forth in SEQ ID NO: 156 or a sequence that exhibits at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 156. In some of any such embodiments, said polynucleotide contains the sequence set forth in SEQ ID NO: 156. In some of any such embodiments, said polynucleotide contains the sequence set forth in SEQ ID NO: 157 or a sequence that exhibits at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 157. In some of any such embodiments, said polynucleotide contains the sequence set forth in SEQ ID NO: 157.

In some of any such embodiments, the polynucleotide further contains a CD33 signal sequence, a GM-CSF signal sequence, a CD8 signal sequence or an Ig kappa signal sequence. In some of any such embodiments, the polynucleotide further contains a CD33 signal sequence. In some of any such embodiments, the CD33 signal sequence is set forth in SEQ ID NO:190 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO:190.

Also provided herein is a vector, containing the polynucleotide of any of the embodiments provided herein. In some embodiments, the vector is a viral vector. In some embodiments, the viral vector is a retroviral vector or a lentiviral vector.

Also provided herein is a cell containing the anti-ROR1 chimeric antigen receptor of any of the embodiments provided herein. Also provided herein is a cell containing the polynucleotide of any of the embodiments provided herein, or the vector of any of the embodiments provided herein. Also provided herein is a cell containing the anti-ROR1 antibody or antigen-binding fragment thereof of the embodiments provided herein, the conjugate of any of the embodiments provided herein, the single chain cell surface protein of any of the embodiments provided herein, the anti-ROR1 chimeric antigen receptor of any of the embodiments provided herein, the polynucleotide of any of the embodiments provided herein, or the vector of any of the embodiments provided herein.

In some embodiments, the cell is a lymphocyte. In some embodiments, the cell is an NK cell or a T cell. In some of any such embodiments, the cell is a T cell and the T cell is a CD4+ or a CD8+ T cell. In some of any such embodiments, the cell is a primary cell obtained from a subject.

In some of any such embodiments, among a plurality of the cells, less than at or about 10%, at or about 9%, at or about 8%, at or about 7%, at or about 5%, at or about 4%, at or about 3%, at or about 2% or at or about 1% of the cells in the plurality contain an anti-ROR1 chimeric antigen receptor that exhibits tonic signaling or antigen independent activity or signaling.

Also provided herein is a composition containing the anti-ROR1 antibody or antigen-binding fragment thereof of any of the embodiments provided herein, the conjugate of any of the embodiments provided herein, the single chain cell surface protein of any of the embodiments provided herein or the anti-ROR1 chimeric antigen receptor of any of the embodiments provided herein.

Also provided herein is a composition containing the cell of any of the embodiments provided herein. In some of any such embodiments, the composition further contains a pharmaceutically acceptable excipient. In some of any such embodiments, the composition contains CD4+ and CD8+ T cells and the ratio of CD4+ to CD8+ T cells is from at or about 1:3 to 3:1. In some of any such embodiments, the composition contains CD4+ and CD8+ T cells and the ratio of CD4+ to CD8+ T cells is at or about 1:2 to 2:1. In some of any such embodiments, the composition contains CD4+ and CD8+ T cells and the ratio of CD4+ to CD8+ T cells is at or about 1:1.

In some of any such embodiments, among a plurality of the cells in the composition, less than at or about 10%, at or about 9%, at or about 8%, at or about 7%, at or about 5%, at or about 4%, at or about 3%, at or about 2% or at or about 1% of the cells in the plurality contain an anti-ROR1 chimeric antigen receptor that exhibits tonic signaling or antigen independent activity or signaling.

Also provided herein is a method of treatment, containing administering the composition of any of the embodiments provided herein to a subject having a disease or disorder associated with ROR1.

Also provided herein is a method of treatment, containing administering the anti-ROR1 antibody or antigen-binding fragment thereof of any of the embodiments provided herein, the conjugate of any of the embodiments provided herein, the single chain cell surface protein of any of the embodiments provided herein, the anti-ROR1 chimeric antigen receptor of any of the embodiments provided herein, the polynucleotide of any of the embodiments provided herein, the vector of any of the embodiments provided herein, or the cell of any of the embodiments provided herein to a subject having a disease or disorder associated with ROR1. Also provided herein is a composition of any of the embodiments provided herein for use in treating a disease or disorder associated with ROR1. Also provided herein is a use of a composition of any of the embodiments provided herein for the manufacture of a medicament for treating a disease or disorder associated with ROR1. Also provided herein is a use of a composition of any of the embodiments provided herein for the treatment of a disease or disorder associated with ROR1. Also provided herein is a method of treatment, containing administering the cells of any of the embodiments provided herein to a subject having a disease or disorder associated with ROR1.

Also provided herein is a method of treatment, containing administering the anti-ROR1 antibody or antigen-binding fragment thereof of any of the embodiments provided herein, the conjugate of any of the embodiments provided herein, the single chain cell surface protein of any of the embodiments provided herein, the anti-ROR1 chimeric antigen receptor of any of the embodiments provided herein, the polynucleotide of any of the embodiments provided herein, the vector of any of the embodiments provided herein, or the cell of any of the embodiments provided herein to a subject having a disease or disorder associated with ROR1. Also provided herein is a cell of any of the embodiments provided herein for use in treating a disease or disorder associated with ROR1. Also provided herein is a use of a cell of any of the embodiments provided herein for the manufacture of a medicament for treating a disease or disorder associated with ROR1. Also provided herein is a use of a cell of any of the embodiments provided herein for the treatment of a disease or disorder associated with ROR1.

In some of any of the provided embodiments, the disease or disorder associated with ROR1 is a cancer. In some of any of the provided embodiments, the cancer is a ROR1-expressing cancer. In some of any of the provided embodiments, the cancer is associated with a ROR1-expressing solid tumor or a ROR1-expressing hematologic malignancy.

In some of any of the provided embodiments, the cancer is associated with a ROR1-expressing solid tumor. In some of any of the provided embodiments, the cancer associated with a solid tumor is selected from the group consisting of neuroblastoma, renal cell carcinoma, colon cancer, colorectal cancer, breast cancer, epithelial squamous cell cancer, melanoma, myeloma, stomach cancer, brain cancer, lung cancer, pancreatic cancer, cervical cancer, ovarian cancer, liver cancer, bladder cancer, prostate cancer, testicular cancer, thyroid cancer, uterine cancer, adrenal cancer and head and neck cancer. In some of any of the provided embodiments, the lung cancer is a non-small cell lung cancer (NSCLC), lung adenocarcinoma, adenocarcinoma, squamous cell carcinoma, small cell carcinoma, and atypical carcinoid. In some of any of the provided embodiments, the lung cancer is a non-small cell lung cancer (NSCLC). In some of any of the provided embodiments, the breast cancer is a triple negative breast cancer (TNBC).

In some of any of the provided embodiments, the cancer is associated with a ROR1-expressing hematologic malignancy. In some of any of the provided embodiments, the hematologic malignancy is selected from the group consisting of B cell leukemia, lymphoma, B cell chronic lymphocytic leukemia (CLL), acute myeloid leukemia (AML), acute lymphocytic leukemia (ALL), Burkitt's Lymphoma or mantle cell lymphoma (MCL).

In some embodiments of the methods provided herein, the disease or disorder associated with ROR1 is a cancer. In some embodiments of the methods provided herein, the cancer is a ROR1-expressing cancer. In some embodiments of the methods provided herein, the cancer is associated with a ROR1-expressing solid tumor or a ROR1-expressing hematologic malignancy.

In some embodiments of the methods provided herein, the cancer is associated with a ROR1-expressing solid tumor. In some embodiments of the methods provided herein, the cancer associated with a solid tumor is selected from the group consisting of neuroblastoma, renal cell carcinoma, colon cancer, colorectal cancer, breast cancer, epithelial squamous cell cancer, melanoma, myeloma, stomach cancer, brain cancer, lung cancer, pancreatic cancer, cervical cancer, ovarian cancer, liver cancer, bladder cancer, prostate cancer, testicular cancer, thyroid cancer, uterine cancer, adrenal cancer and head and neck cancer. In some embodiments of the methods provided herein, the lung cancer is a non-small cell lung cancer (NSCLC), lung adenocarcinoma, adenocarcinoma, squamous cell carcinoma, small cell carcinoma, and atypical carcinoid. In some embodiments of the methods provided herein, the lung cancer is a non-small cell lung cancer (NSCLC). In some embodiments of the methods provided herein, the breast cancer is a triple negative breast cancer (TNBC).

In some embodiments of the methods provided herein, the cancer is associated with a ROR1-expressing hematologic malignancy. In some embodiments of the methods provided herein, the hematologic malignancy is selected from the group consisting of B cell leukemia, lymphoma, B cell chronic lymphocytic leukemia (CLL), acute myeloid leukemia (AML), acute lymphocytic leukemia (ALL), Burkitt's Lymphoma or mantle cell lymphoma (MCL).

In some embodiments of the cells or compositions for use provided herein, the disease or disorder associated with ROR1 is a cancer. In some embodiments of the cells or compositions for use provided herein, the cancer is a ROR1-expressing cancer. In some embodiments of the cells or compositions for use provided herein, the cancer is associated with a ROR1-expressing solid tumor or a ROR1-expressing hematologic malignancy.

In some embodiments of the cells or compositions for use provided herein, the cancer is associated with a ROR1-expressing solid tumor. In some embodiments of the cells or compositions for use provided herein, the cancer associated with a solid tumor is selected from the group consisting of neuroblastoma, renal cell carcinoma, colon cancer, colorectal cancer, breast cancer, epithelial squamous cell cancer, melanoma, myeloma, stomach cancer, brain cancer, lung cancer, pancreatic cancer, cervical cancer, ovarian cancer, liver cancer, bladder cancer, prostate cancer, testicular cancer, thyroid cancer, uterine cancer, adrenal cancer and head and neck cancer. In some embodiments of the cells or compositions for use provided herein, the lung cancer is a non-small cell lung cancer (NSCLC), lung adenocarcinoma, adenocarcinoma, squamous cell carcinoma, small cell carcinoma, and atypical carcinoid. In some embodiments of the cells or compositions for use provided herein, the lung cancer is a non-small cell lung cancer (NSCLC). In some embodiments of the cells or compositions for use provided herein, the breast cancer is a triple negative breast cancer (TNBC).

In some embodiments of the cells or compositions for use provided herein, the cancer is associated with a ROR1-expressing hematologic malignancy. In some embodiments of the cells or compositions for use provided herein, the hematologic malignancy is selected from the group consisting of B cell leukemia, lymphoma, B cell chronic lymphocytic leukemia (CLL), acute myeloid leukemia (AML), acute lymphocytic leukemia (ALL), Burkitt's Lymphoma or mantle cell lymphoma (MCL).

In some embodiments of the uses provided herein, the disease or disorder associated with ROR1 is a cancer. In some embodiments of the uses provided herein, the cancer is a ROR1-expressing cancer. In some embodiments of the uses provided herein, the cancer is associated with a ROR1-expressing solid tumor or a ROR1-expressing hematologic malignancy.

In some embodiments of the uses provided herein, the cancer is associated with a ROR1-expressing solid tumor. In some embodiments of the uses provided herein, the cancer associated with a solid tumor is selected from the group consisting of neuroblastoma, renal cell carcinoma, colon cancer, colorectal cancer, breast cancer, epithelial squamous cell cancer, melanoma, myeloma, stomach cancer, brain cancer, lung cancer, pancreatic cancer, cervical cancer, ovarian cancer, liver cancer, bladder cancer, prostate cancer, testicular cancer, thyroid cancer, uterine cancer, adrenal cancer and head and neck cancer. In some embodiments of the uses provided herein, the lung cancer is a non-small cell lung cancer (NSCLC). In some embodiments of the uses provided herein, the breast cancer is a triple negative breast cancer (TNBC).

In some embodiments of the uses provided herein, the cancer is associated with a ROR1-expressing hematologic malignancy. In some embodiments of the uses provided herein, the hematologic malignancy is selected from the group consisting of B cell leukemia, lymphoma, B cell chronic lymphocytic leukemia (CLL), acute myeloid leukemia (AML), acute lymphocytic leukemia (ALL), Burkitt's Lymphoma or mantle cell lymphoma (MCL).

In some of any such embodiments, the disease or disorder associated with ROR1 is associated with ROR1 expression. In some of any such embodiments, the disease or disorder associated with ROR1 is a B cell-related disorder. In some of any such embodiments, the disease or disorder associated with ROR1 is a cancer. In some of any such embodiments, the cancer is a ROR1-expressing cancer. In some of any such embodiments, the ROR1-expressing cancer is selected from the group consisting of B cell leukemia, lymphoma, B cell chronic lymphocytic leukemia (CLL), acute myeloid leukemia (AML), acute lymphocytic leukemia (ALL), Burkitt's Lymphoma, mantle cell lymphoma (MCL), non-small cell lung cancer (NSCLC), neuroblastoma, renal cell carcinoma, colon cancer, colorectal cancer, breast cancer, epithelial squamous cell cancer, melanoma, myeloma, stomach cancer, brain cancer, lung cancer, pancreatic cancer, cervical cancer, ovarian cancer, liver cancer, bladder cancer, prostate cancer, testicular cancer, thyroid cancer, uterine cancer, adrenal cancer and head and neck cancer.

Also provided herein is a kit containing the anti-ROR1 antibody or antigen-binding fragment thereof of any of the embodiments provided herein, the single chain cell surface protein of any of the embodiments provided herein, the conjugate of any of the embodiments provided herein, the anti-ROR1 chimeric antigen receptor of any of the embodiments provided herein, the cell of any of the embodiments provided herein, or the composition of any of the embodiments provided herein, and instructions for use. In some of any such embodiments, the instructions are for administering the anti-ROR1 antibody or antigen-binding fragment thereof, the single chain cell surface protein, the conjugate, the anti-ROR1 chimeric antigen receptor, the cell or the composition. In some of any such embodiments, the instructions are in accord with the method, the composition for use or the use of any of the embodiments provided herein.

Also provided herein is an article of manufacture containing the anti-ROR1 antibody or antigen-binding fragment thereof of any of the embodiments provided herein, the single chain cell surface protein of any of the embodiments provided herein, the conjugate of any of the embodiments provided herein, the anti-ROR1 chimeric antigen receptor of any of the embodiments provided herein, the cell of any of the embodiments provided herein, the composition of any of the embodiments provided herein, or the kit of any of the embodiments provided herein.

### BRIEF DESCRIPTION OF THE DRAWINGS

FIG. 1 depicts the relative intracellular IFN-γ, TNF-α or IL-2 expression levels by ICS in GFP+CD4+ T cells compared to the levels of each cytokine in cells expressing the reference anti-ROR1 (R12) CAR, after 24 hours stimulation with plate-bound recombinant ROR1-Fe, in primary T cells expressing select candidate anti-ROR1 CAR-expressing T cells from a screen as described in Example 1.

FIG. 2 shows the relative intracellular IFN-γ, TNF-α or IL-2 expression levels by ICS in primary T cells expressing select candidate anti-ROR1 CAR-expressing T cells from a screen as described in Example 1, compared to the levels of each cytokine in cells expressing the reference anti-ROR1 (R12) CAR, after 24-70 hour co-culture with MDA-MB-231 target cells expressing ROR1.

FIGS. 3A-3B depict the results from an in vitro cytotoxicity assay, after co-culture of NucLight Red (NLR)-labeled H1975 target cells and primary T cells expressing one of 6 selected candidate anti-ROR1 CARs, as assessed by measuring the loss of red fluorescent signal over a period of between 0 and 70 hours. As controls, cultures of target cells only and co-cultures of mock cells (not expressing a CAR) with the target cells were assessed.

FIGS. 4A-4C show the production of IFN-γ, TNF-α or IL-2 after 70 hours of co-culture of primary T cells expressing one of 6 selected candidate anti-ROR1 CARs from two separate donors, with H1975 (FIGS. 4A and 4B), or the production of IL-2 after co-culture with MDA-MB-231, A549 or BT-549 (FIG. 4C) target cells. As controls, cultures of target cells only and co-cultures of mock cells (not expressing a CAR) with the target cells were assessed.

FIGS. 5A-5D show the anti-tumor activity, as assessed by the changes in mean or individual tumor volume after administration of cells expressing one of 6 selected candidate anti-ROR1 CARs or the reference anti-ROR1 (R12) CAR, in a H1975 non-small cell lung cancer (NSCLC) mouse model.

FIG. 5A (low dose) and FIG. 5B (high dose) depict mean tumor volume of all treated mice; in this depiction, tumor curves were terminated after the first mouse of a group succumbed to disease. Results from all individual mice are shown in FIG. 5C (low dose) or FIG. 5D (high dose). As a control, mice were administered cells not expressing a CAR (mock) or were untreated.

FIGS. 6A-6B depict the Kaplan-Meier survival curves after administration of a low dose (FIG. 6A) or a high dose (FIG. 6B) of cells expressing one of 6 selected candidate anti-ROR1 CARs or the reference anti-ROR1 (R12) CAR, in a H1975 non-small cell lung cancer (NSCLC) mouse model.

FIGS. 7A-7D show the average number of CD4+ and CD8+ CAR-expressing cells in the blood of each mouse determined at day 10 and day 24 after administration of cells expressing one of 6 selected candidate anti-ROR1 CARs or the reference anti-ROR1 (R12) CAR, at the low dose (FIGS. 7A and 7B) or high dose (FIGS. 7C and 7D). FIGS. 7E-7F show the average number of CD3+ CAR-expressing cells in the blood of each mouse determined at day 10 and day 24 after administration of cells expressing one of 6 selected candidate anti-ROR1 CARs or the reference anti-ROR1 (R12) CAR, at the low dose (FIG. 7E) or high dose (FIG. 7F). Data are shown as individual values along with group means±standard deviation.

FIGS. 8A-8C depict the changes in the mean and individual tumor volume in H1975 non-small cell lung cancer (NSCLC) mice administered cells expressing anti-ROR1 CAR-F, CAR-A or the R12 reference CAR, at the low dose (mean: FIG. 8A; individual: FIG. 8C) or the high dose (mean: FIG. 8B; individual: FIG. 8C). As a control, mice were administered cells not expressing a CAR (mock) or were untreated.

FIGS. 9A-9B depict the Kaplan-Meier survival curves after administration of a low dose (FIG. 9A) or a high dose (FIG. 9B) of mice administered cells expressing anti-ROR1 CAR-F, CAR-A or the R12 reference CAR. As a control, mice were administered cells not expressing a CAR (mock) or were untreated.

FIGS. 10A-10D depict the average CD4+(FIGS. 10A and 10C) and CD8+ (FIGS. 10B and 10D) CAR+ T cell count per microliter of blood at days 7, 14 and 21 after administration of a low dose (FIGS. 10A and 10B) or a high dose (FIGS. 10C and 10D) cells expressing anti-ROR1 CAR-F, CAR-A or the R12 reference CAR.

FIGS. 11A-11B depict the number of CD4+(FIG. 11A) and CD8+ (FIG. 11B) CAR+ T cells present in the tumor at 14 days after administration of cells expressing anti-ROR1 CAR-F, CAR-A or the R12 reference CAR.

FIGS. 12A-12C depict bioluminescence images assessed up to approximately day 49 post administration of a low dose (FIG. 12B) or a high dose (FIG. 12C) of cells expressing anti-ROR1 CAR-F, CAR-A or the R12 reference CAR in MDA-MB-231 triple negative breast cancer mouse model. As a control, mice were administered cells not expressing a CAR (mock) or were untreated (FIG. 12A).

FIGS. 13A-13C depict the mean measured total flux (p/s) from the bioluminescence imaging, assessed up to approximately day 49 post administration of a low dose (FIG. 13A) or a high dose (FIG. 13B) of cells expressing anti-ROR1 CAR-F, CAR-A or the R12 reference CAR in MDA-MB-231 triple negative breast cancer mouse model, shown as group means±standard error. FIG. 13C depicts the measured total flux (p/s) of individual mice. As a control, mice were administered cells not expressing a CAR (mock) or were untreated.

FIGS. 14A-14E depict the changes in the mean and individual tumor volume in MDA-MB-231 triple negative breast cancer model mice administered cells expressing anti-ROR1 CAR-F, CAR-A or the R12 reference CAR, at the low dose (mean: FIG. 14A) or the high dose (mean: FIG. 14B). The changes in the mean tumor volume following administration of T cells expressing anti-ROR1 CAR F and reference CAR R12, up to a further time point in the same study are depicted for the high dose (FIG. 14C) and low dose (FIG. 14D). Results of tumor volume for individual treated mice at the high dose or low dose for each treated condition are shown in FIG. 14E. Mean tumor volume is depicted as group means±standard error up to the last day that all mice in treatment groups survived. As a control, mice were administered cells not expressing a CAR (mock) or were untreated.

FIGS. 15A and 15B depict the Kaplan-Meier survival curve in MDA-MB-231 triple negative breast cancer model mice administered cells expressing anti-ROR1 CAR-F or the R12 reference CAR, at the low dose (FIG. 15B) or the high dose (FIG. 15A). As a control, mice were administered cells not expressing a CAR (mock) or were untreated.

FIGS. 16A-16D show the average number of CD4+(FIGS. 16A and 16C) and CD8+ (FIGS. 16B and 16D) CAR-expressing cells in the blood of the animal were determined at days 7, 14, 21 and 30 after administration after administration of cells expressing anti-ROR1 CAR-F, CAR-A or the R12 reference CAR, at the low dose (FIGS. 16A and 16B) or a high dose (FIGS. 16C and 16D). The average number of CD3+ CAR-expressing cells (CD45+CD3+ CAR+) in the blood of each mouse administered cells expressing anti-ROR1 CAR-F or the R12 reference CAR also was determined at days 7, 14, 21 and 30 are shown in FIG. 16E. As shown, cells expressing anti-ROR1 CAR-F exhibited high expansion in the MDA-MB-231 mouse model, when administered at both high and low doses.

FIG. 17 depicts the loss of Rapid Red labeled target cells (CD4/CD8 T cell depleted leukapheresis samples) co-cultured for approximately 6 days with engineered cells expressing anti-ROR1 CAR-F, anti-CD19 CAR or the R12 reference CAR generated from with from two primary CLL donors, at two E:T ratios.

FIG. 18A shows the results of a cytotoxicity assay by flow cytometry, showing the loss of CD19+ target cells or CD19+ ROR1+ target cells (CD4/CD8 T cell depleted leukapheresis samples) co-cultured for approximately 4 days with engineered cells expressing anti-ROR1 CAR-F, anti-CD19 CAR or the R12 reference CAR generated from two primary CLL donors, at two E:T ratios. FIG. 18B depicts the proliferation of CAR-expressing cells (anti-ROR1 CAR-F, anti-CD19 CAR or the R12 reference CAR) labeled with CellTrace™ Violet (CTV) cell proliferation reagent, and co-cultured with target cells from the subjects with CLL at an E:T ratio of 2.5:1 and 0.25:1. FIG. 18C shows the production of IFN-γ, TNF-α and IL-2, as assessed from the supernatant of the co-culture on day 4. Mock treated cells or target cells only were compared as controls.

FIGS. 19A-19D depict the changes in the mean and individual tumor volume in a mouse model of mantel cell lymphoma (MCL), implanted with firefly luciferase and green fluorescent protein (FfLuc-GFP)-expressing human mantel cell lymphoma (MCL) JeKo-1 cells administered cells expressing anti-ROR1 CAR-F, anti-CD19 or the R12 reference CAR, at the high dose (mean: FIG. 19A; individual: FIG. 19C) or the low dose (mean: FIG. 19B; individual: FIG. 19D) or. As a control, mice were administered cells not expressing a CAR (mock) or were untreated.

FIGS. 20A-20B depict the Kaplan-Meier survival curve in a mouse model of MCL administered cells expressing anti-ROR1 CAR-F, anti-CD19 or the R12 reference CAR, at the high dose (FIG. 20A) or the low dose (FIG. 20B). As a control, mice were administered cells not expressing a CAR (mock) or were untreated.

FIG. 21 shows the average number of average number of CD3+ CAR-expressing cells in the blood of a mouse model of MCL, determined at days 7, 14, 21 and 28 after administration of engineered cells expressing anti-ROR1 CAR-F, anti-CD19 or the R12 reference CAR, at the low dose or a high dose.

FIGS. 22A-22E depict binding of various recombinantly produced anti-ROR1 scFv-mFcs, including anti-ROR1 scFv ROR1-1 (SEQ ID NO: 118; FIG. 22A), ROR1-2 (SEQ ID NO: 127; FIG. 22B), ROR1-3 (SEQ ID NO: 109; FIG. 22C) and ROR1-4 (SEQ ID NO: 134; FIG. 22D), the scFv binding domains of CAR-A, CAR-F, CAR-G, CAR-I, CAR-R and CAR-B1, and the scFv antigen binding domain of the reference CAR R12 (SEQ ID NO: 142; FIG. 22E), to with a C-terminal 6×His tag (ROR1 ECD 6×His), assessed by surface plasmon resonance (SPR) using multi-cycle kinetics implemented by subsequent injections of recombinant human ROR1 at concentrations of 183, 61.0, 20.33, 6.78, and 2.26 nM.

FIG. 23 depicts ROR1 expression in response to a 2-fold increasing dilution series of doxycycline from 512 ng/mL to 0 ng/mL in a K562-ROR1-TetOn cell line. The ROR1-parental K562 cells, two endogenously expressing ROR1+ cell lines, the MCL cell line JeKo-1 and the TNBC cell line MDA-MB-231 were used as controls.

FIG. 24 depicts a dose response curve for production of IFNγ, IL-2 and TNFα assessed from supernatants of a 72 hour co-culture of engineered cells expressing anti-ROR1 CAR-F or the reference CAR R12, with K562-ROR1-TetOn at a 4:1 E:T ratio with the addition of increasing concentrations of doxycycline. Cells not expressing a CAR (mock) were used as a control.

FIGS. 25A-25B depict results of a cytotoxicity assay, as determined by loss of red fluorescent signal from NucLight Red-labeled K562-ROR1-TetOn cells cultured in the presence of various concentrations (2 ng/mL-512 ng/mL) of doxycycline and of engineered cells expressing anti-ROR1 CAR-F or the reference CAR R12, at a 4:1 E:T ratio, for approximately 72 hours. DMSO, no doxycycline and untreated cells were used as controls.

FIG. 26 shows assessment of ROR1 species cross reactivity in Jurkat Nur77 reporter cells expressing either the ROR1 CAR-F or the ROR1 R12 CAR co-cultured with CT26 cells engineered to express human ROR1 (hROR1) or murine ROR1 (mROR1). K562 cells and unmodified CT26 cells were used as non-specific controls.

### DETAILED DESCRIPTION

Provided are receptor tyrosine kinase-like orphan receptor 1 (ROR1)-binding molecules, such as antibodies (including antigen-binding antibody fragments, such as single chain fragments, including single chain Fv fragments (scFvs)) and recombinant receptors, including chimeric receptors containing such antibodies or fragments and nucleic acids encoding such antibodies, fragments or recombinant receptors. In some aspects, provided are antibodies, fragments and chimeric antigen receptors (CARs) targeting or directed to ROR1 and ROR1-expressing cells and disease. It is observed that ROR1 is expressed in cells or tissues associated with certain diseases and conditions such as malignancies, e.g., on malignant plasma cells such as from relapsed or newly diagnosed myeloma patients, for example, with little expression on normal tissues. Among the provided embodiments are approaches useful in the treatment of diseases and conditions and/or for targeting such cell types, including nucleic acid molecules that encode ROR1-binding antibodies, fragments or receptors and the encoded antibodies or antigen-binding fragments and receptors. Also provided are compositions and articles of manufacture comprising the same. The receptors generally can contain antibodies (including antigen-binding antibody fragments, such as heavy chain variable (V_{H}) regions, single domain antibody fragments and single chain fragments, including scFvs) specific for ROR1, for example as the antigen-binding domain. Also provided are cells, such as engineered or recombinant cells, expressing such ROR1-binding receptors, e.g., anti-ROR1 CARs and/or containing nucleic acids encoding such receptors, and compositions and articles of manufacture and therapeutic doses containing such cells. Also provided are methods of making and using the antibodies and fragments as well as cells expressing or containing the antibodies and fragments, such as for production of the antibodies or fragments thereof. Also provided are compositions, including pharmaceutical compositions, containing such antibodies, antigen-binding fragments, receptors or cells, and conjugates comprising such antibodies or fragments. In some aspects, the provided compositions, antibodies, antigen-binding fragments, receptors or cells can be used in connection with a therapy or a method of treatment.

Therapies targeting ROR1, such as with anti-ROR1 antibodies or adoptive cell therapies (including those involving the administration of cells expressing chimeric receptors such as chimeric antigen receptors (CARs) and/or other recombinant antigen receptors, specific for ROR1, as well as other adoptive immune cell and adoptive T cell therapies) can be effective in the treatment of cancer and other diseases and disorders, for example, ROR1. In certain contexts, available approaches to adoptive cell therapy may not always be entirely satisfactory. In some aspects, the ability of the administered cells to recognize and bind to a target, e.g., target antigen such as ROR1, to traffic, localize to and successfully enter appropriate sites within the subject, tumors, and environments thereof, to become activated, expand, to exert various effector functions, including cytotoxic killing and secretion of various factors such as cytokines, to persist, including long-term, to differentiate, transition or engage in reprogramming into certain phenotypic states to provide effective and robust recall responses following clearance and re-exposure to target ligand or antigen, and avoid or reduce exhaustion, anergy, terminal differentiation, and/or differentiation into a suppressive state.

In some contexts, properties of particular target antigens that the antibodies or recombinant receptors containing antigen-binding domains specifically bind, recognize or target, can that affect the activity of the receptor. In some contexts, ROR1 is expressed by certain cancers and is an attractive therapeutic target for cell therapy. Improved strategies are needed for optimal responses to antibody or cell therapies, in particular, for recombinant receptors that specifically bind, recognize or target ROR1. Provided are embodiments that meet such needs.

In some aspects, the provided embodiments are based on observations that administration of engineered cells expressing the provided ROR1 binding molecules, such as chimeric antigen receptors (CARs), exhibit an improved antigen-specific activity, signaling and function, high anti-tumor activity, consistent antigen-dependent activity or signaling, greater or prolonged in vivo expansion, and improved persistence when administered, while exhibiting minimal antigen-independent activity or signaling or cross-reactivity to a different antigen. Such antibodies and recombinant receptors can be used to facilitate safe and effective treatment of particular diseases and disorders, such as those associated with expression of ROR1.

In some contexts, optimal response to therapy can depend on the ability of the antibody or antigen-binding fragment, or recombinant receptors that contain such antibody or antigen-binding fragment as antigen-binding domains, to recognize the target antigen. In some aspects, it is observed herein that the binding affinity, specificity or kinetics of binding to ROR1 of certain provided antibody or antigen-binding fragments, such as when present as the antigen-binding domain of a recombinant receptor (e.g. CAR), can be associated with an improved or greater response to the therapy. In some aspects, the provided embodiments are based on observations that provided binding molecules exhibit a lower binding affinity and/or a faster dissociation rate constant (k_{off} or k_{d}; fast off-rate) to ROR1 compared to available antibodies, and also exhibit improved anti-tumor activity, greater or prolonged in vivo expansion, improved persistence, and/or reduced antigen-independent activity or signaling. In some aspects, engineered cells (e.g. T cells) expressing a recombinant receptor containing an antigen-binding domain (e.g., antibody or antigen-binding fragment thereof) with a lower binding affinity (e.g. higher equilibrium dissociation constant) may exhibit substantially improved in vivo expansion, increased persistence, greater or improved antigen-specific anti-tumor activity and prolonged survival, including against various different types of tumors.

In some aspects, engineered cells (e.g. T cells) expressing a recombinant receptor containing an antigen-binding domain (e.g., antibody or antigen-binding fragment thereof) with a faster dissociation rate constant (k_{off} or k_{d}; fast off-rate) may exhibit substantially improved in vivo expansion, increased persistence, greater or improved antigen-specific anti-tumor activity and prolonged survival, including against various different types of tumors. In some aspects, engineered cells (e.g. T cells) expressing a recombinant receptor containing an antigen-binding domain (e.g., antibody or antigen-binding fragment thereof) with a lower binding affinity (e.g. higher equilibrium dissociation constant) and a faster dissociation rate constant (k_{off} or k_{d}; fast off-rate) may exhibit substantially improved in vivo expansion, increased persistence, greater or improved antigen-specific anti-tumor activity and prolonged survival, including against various different types of tumors. Without wishing to be bound by theory, in some aspects, it is observed herein that the increased antigen-binding off-rate of the binding domain of an exemplary anti-ROR1 CAR for ROR1 binding potentially contributes to increased sensitivity of the CAR to low antigen levels.

In some contexts, optimal response to therapy such as cell therapy can depend on the ability of the engineered recombinant receptors such as CARs, to be consistently and reliably expressed on the surface of the cells and/or bind the target antigen. For example, in some cases, heterogeneity of the transcribed RNA from an introduced transgene (e.g., encoding the recombinant receptor) can affect the expression and/or activity of the recombinant receptor, in some cases when expressed in a cell, such as a human T cell, used in cell therapy.

In some contexts, the length and type of spacer in the recombinant receptor, such as a CAR, can affect the expression, activity and/or function of the receptor.

Also, in some contexts, certain recombinant receptors can exhibit antigen-independent activity or signaling (also known as “tonic signaling”), which could lead to undesirable effects, such as due to increased differentiation and/or exhaustion of T cells that express the recombinant receptor. In some aspects, such activities may limit the T cell's activity, effect or potency. In some cases, during engineering and ex vivo expansion of the cells for recombinant receptor expression, the cells may exhibit phenotypes indicative of exhaustion, due to tonic signaling through the recombinant receptor.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. ROR1-BINDING MOLECULES

Provided in some aspects are ROR1-binding molecules, such as ROR1-binding polypeptides. Such binding molecules include antibodies (including antigen-binding fragments) that specifically bind to ROR1 proteins, such as a human ROR1 protein. Also among the binding molecules are polypeptides containing such antibodies, including single chain cell surface proteins, e.g., recombinant receptors such as chimeric antigen receptors (CARs), containing such antibodies. Provided in some aspects are ROR1-binding cell surface proteins, such as recombinant receptors or chimeric antigen receptors (CARs) that bind ROR1 molecules and polynucleotides encoding ROR1 binding cell surface proteins, such as recombinant receptors (e.g., CARs), and cells expressing such receptors. Also provided are polynucleotides containing nucleic acids sequences encoding all or a portion of such antibodies, antigen-binding fragments and binding molecules, such as those described in Section I.A or I.D. Exemplary of such polynucleotides include those described in Section I.E. In some aspects the polynucleotides can be introduced into a cell to generate an engineered cell that contains or expresses the provided binding molecules, e.g., ROR1-binding antibodies, antigen-binding domains and receptors, such as CARs.

### A. ROR1-Targeting Antibodies

Provided are anti-ROR1 polypeptides, including antibodies and functional antigen-binding fragments. Among the ROR1-binding polypeptides are antibodies, such as single-chain antibodies (e.g., antigen binding antibody fragments), such as those containing a heavy chain variable (V_{H}) region and/or a light chain variable (V_{L}) region, or a portion thereof. In some embodiments, the antibodies or antigen-binding fragments include a V_{H} and a V_{L}, such as single chain Fv fragments (scFvs). The antibodies include antibodies that specifically bind to ROR1, e.g., human ROR1. Among the provided anti-ROR1 antibodies are human antibodies, or antibodies that are modified from or variant of human antibodies. The antibodies include isolated antibodies. Also provided are ROR1-binding molecules containing such antibodies, such as single-chain proteins, fusion proteins, conjugates and/or recombinant receptors such as chimeric receptors, including antigen receptors. In some aspects, the ROR1-binding molecules include isolated molecules.

Also provided are ROR1-binding cell surface proteins, such as ROR1-binding recombinant receptors. The ROR1-binding cell surface proteins can contain the provided antibodies (e.g., antigen-binding antibody fragments) that specifically bind to ROR1, such as to ROR1 proteins, such as human ROR1 protein. In some aspects, the provided binding molecules bind to an extracellular portion of ROR1. In some examples, the recombinant receptors are chimeric antigen receptors, such as those containing anti-ROR1 antibodies or antigen-binding fragments thereof.

Also provided are polynucleotides containing nucleic acids sequences encoding all or a portion of such antibodies, antigen-binding fragments and binding molecules. The provided polynucleotides can be incorporated into constructs, such as deoxyribonucleic acid (DNA) or RNA constructs, such as those that can be introduced into cells for expression of the encoded ROR1-binding antibodies, antigen-binding fragments, conjugates or receptors, e.g., anti-ROR1 CARs. In some aspects, the encoded antibodies, antigen-binding fragments, conjugates and receptors, such as those containing ROR1-binding polypeptides, and compositions and articles of manufacture and uses of the same, also are provided.

The term “antibody” herein is used in the broadest sense and includes polyclonal and monoclonal antibodies, including intact antibodies and functional (antigen-binding) antibody fragments, including fragment antigen binding (Fab) fragments, F(ab′)₂ fragments, Fab′ fragments, Fv fragments, recombinant IgG (rIgG) fragments, heavy chain variable (V_{H}) regions capable of specifically binding the antigen, single chain antibody fragments, including single chain variable fragments (scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific or trispecific, antibodies, diabodies, triabodies, and tetrabodies, tandem di-scFv, tandem tri-scFv. Unless otherwise stated, the term “antibody” should be understood to encompass functional antibody fragments thereof also referred to herein as “antigen-binding fragments.” The term also encompasses intact or full-length antibodies, including antibodies of any class or sub-class, including IgG and sub-classes thereof, IgM, IgE, IgA, and IgD.

The terms “complementarity determining region,” and “CDR,” synonymous with “hypervariable region” or “HVR,” are known to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4).

The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme); A1-Lazikani et al., (1997) JMB 273,927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 Jan.; 27(1):55-77 (“IMGT” numbering scheme); Honegger A and Plückthun A, “Yet another numbering scheme for immunoglobulin variable domains: an automatic modeling and analysis tool,” J Mol Biol, 2001 Jun. 8; 309(3):657-70, (“Aho” numbering scheme); Martin et al., “Modeling antibody hypervariable loops: a combined algorithm,” PNAS, 1989, 86(23):9268-9272, (“AbM” numbering scheme); and Ye et al., “IgBLAST: an immunoglobulin variable domain sequence analysis tool,” Nucleic Acids Res. 2013 July; 41 (Web Server issue):W34-40, (“IgBLAST numbering scheme).

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme. The AbM scheme is a compromise between Kabat and Chothia definitions based on that used by Oxford Molecular's AbM antibody modeling software. The IgBLAST scheme is based on matching to germline V, D and J genes, and can be determined using National Center for Biotechnology Information (NCBI)'s IgBLAST tool.

Table 1, below, lists exemplary position boundaries of CDR-L1, CDR-L2, CDR-L3 and CDR-H1, CDR-H2, CDR-H3 as identified by Kabat, Chothia, AbM, and Contact schemes, respectively. For CDR-H1, residue numbering is listed using both the Kabat and Chothia numbering schemes. FRs are located between CDRs, for example, with FR-L1 located before CDR-L1, FR-L2 located between CDR-L1 and CDR-L2, FR-L3 located between CDR-L2 and CDR-L3 and so forth. It is noted that because the shown Kabat numbering scheme places insertions at H35A and H35B, the end of the Chothia CDR-H1 loop when numbered using the shown Kabat numbering convention varies between H32 and H34, depending on the length of the loop.

TABLE 1
Boundaries of CDRs according to various numbering schemes.

| CDR | Kabat | Chothia | AbM | Contact |
|---|---|---|---|---|
| CDR-L1 | L24--L34 | L24--L34 | L24--L34 | L30--L36 |
| CDR-L2 | L50--L56 | L50--L56 | L50--L56 | L46--L55 |
| CDR-L3 | L89--L97 | L89--L97 | L89--L97 | L89--L96 |
| CDR-H1 | H31--H35B | H26--H32.34 | H26--H35B | H30--H35B |
| (Kabat | | | | |
| Numbering¹) | | | | |
| CDR-H1 | H31--H35 | H26--H32 | H26--H35 | H30--H35 |
| (Chothia | | | | |
| Numbering²) | | | | |
| CDR-H2 | H50--H65 | H52--H56 | H50--H58 | H47--H58 |
| CDR-H3 | H95--H102 | H95--H102 | H95--H102 | H93--H101 |

¹Kabat et al. (1991), “Sequences of Proteins of Immunological Interest,” 5th Ed. Public Health Service, National Institutes of Health, Bethesda, MD
²Al-Lazikani et al., (1997) JMB 273, 927-948

Thus, unless otherwise specified, a “CDR” or “complementary determining region,” or individual specified CDRs (e.g., CDR-H1, CDR-H2, CDR-H3), of a given antibody or region thereof, such as a variable region thereof, should be understood to encompass a (or the specific) complementary determining region as defined by any of the aforementioned schemes, or other known schemes. For example, where it is stated that a particular CDR (e.g., a CDR-H3) contains the amino acid sequence of a corresponding CDR in a given V_{H} or V_{L} region amino acid sequence, it is understood that such a CDR has a sequence of the corresponding CDR (e.g., CDR-H3) within the variable region, as defined by any of the aforementioned schemes, or other known schemes. In some embodiments, specific CDR sequences are specified. Exemplary CDR sequences of provided antibodies are described using various numbering schemes (see e.g. Table 2), although it is understood that a provided antibody can include CDRs as described according to any of the other aforementioned numbering schemes or other known numbering schemes.

Likewise, unless otherwise specified, a FR or individual specified FR(s) (e.g., FR-H1, FR-H2, FR-H3, FR-H4), of a given antibody or region thereof, such as a variable region thereof, should be understood to encompass a (or the specific) framework region as defined by any of the known schemes. In some instances, the scheme for identification of a particular CDR, FR, or FRs or CDRs is specified, such as the CDR as defined by the Kabat, Chothia, AbM, IgBLAST, IMGT, or Contact method, or other known schemes. In other cases, the particular amino acid sequence of a CDR or FR is given.

The term “variable region” or “variable domain” refers to the domain of an antibody heavy or light chain that is involved in binding the antibody to antigen. The variable regions of the heavy chain and light chain (V_{H} and V_{L}, respectively) of a native antibody generally have similar structures, with each domain comprising four conserved framework regions (FRs) and three CDRs. (See, e.g., Kindt et al. Kuby Immunology, 6th ed., W.H. Freeman and Co., page 91 (2007). A single V_{H} or V_{L} domain may be sufficient to confer antigen-binding specificity. Furthermore, antibodies that bind a particular antigen may be isolated using a V_{H} or V_{L} domain from an antibody that binds the antigen to screen a library of complementary V_{L} or V_{H} domains, respectively. See, e.g., Portolano et al., J. Immunol. 150:880-887 (1993); Clarkson et al., Nature 352:624-628 (1991).

Among the provided antibodies are antibody fragments. An “antibody fragment” or “antigen-binding fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; heavy chain variable (V_{H}) regions, single-chain antibody molecules such as scFvs and single-domain antibodies comprising only the V_{H} region; and multispecific antibodies formed from antibody fragments. In some embodiments, the antibody is or comprises an antibody fragment comprising a variable heavy chain (V_{H}) and a variable light chain (V_{L}) region. In particular embodiments, the antibodies are single-chain antibody fragments comprising a heavy chain variable (V_{H}) region and/or a light chain variable (V_{L}) region, such as scFvs.

Single-domain antibodies (sdAbs) are antibody fragments comprising all or a portion of the heavy chain variable region or all or a portion of the light chain variable region of an antibody. In certain embodiments, a single-domain antibody is a human single-domain antibody.

Antibody fragments can be made by various techniques, including but not limited to proteolytic digestion of an intact antibody as well as production by recombinant host cells. In some embodiments, the antibodies are recombinantly-produced fragments, such as fragments comprising arrangements that do not occur naturally, such as those with two or more antibody regions or chains joined by synthetic linkers, e.g., peptide linkers, and/or that are may not be produced by enzyme digestion of a naturally-occurring intact antibody. In some aspects, the antibody fragments are scFvs.

A “humanized” antibody is an antibody in which all or substantially all CDR amino acid residues are derived from non-human CDRs and all or substantially all FR amino acid residues are derived from human FRs. A humanized antibody optionally may include at least a portion of an antibody constant region derived from a human antibody. A “humanized form” of a non-human antibody, refers to a variant of the non-human antibody that has undergone humanization, typically to reduce immunogenicity to humans, while retaining the specificity and affinity of the parental non-human antibody. In some embodiments, some FR residues in a humanized antibody are substituted with corresponding residues from a non-human antibody (e.g., the antibody from which the CDR residues are derived), e.g., to restore or improve antibody specificity or affinity.

Among the provided anti-ROR1 antibodies are human antibodies. A “human antibody” is an antibody with an amino acid sequence corresponding to that of an antibody produced by a human or a human cell, or non-human source that utilizes human antibody repertoires or other human antibody-encoding sequences, including human antibody libraries. The term excludes humanized forms of non-human antibodies comprising non-human antigen-binding regions, such as those in which all or substantially all CDRs are non-human. The term includes antigen-binding fragments of human antibodies.

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic animals, the endogenous immunoglobulin loci have generally been inactivated. Human antibodies also may be derived from human antibody libraries, including phage display and cell-free libraries, containing antibody-encoding sequences derived from a human repertoire.

Among the provided antibodies are monoclonal antibodies, including monoclonal antibody fragments. The term “monoclonal antibody” as used herein refers to an antibody obtained from or within a population of substantially homogeneous antibodies, i.e., the individual antibodies comprising the population are identical, except for possible variants containing naturally occurring mutations or arising during production of a monoclonal antibody preparation, such variants generally being present in minor amounts. In contrast to polyclonal antibody preparations, which typically include different antibodies directed against different epitopes, each monoclonal antibody of a monoclonal antibody preparation is directed against a single epitope on an antigen. The term is not to be construed as requiring production of the antibody by any particular method. A monoclonal antibody may be made by a variety of techniques, including but not limited to generation from a hybridoma, recombinant DNA methods, phage-display and other antibody display methods.

The terms “polypeptide” and “protein” are used interchangeably to refer to a polymer of amino acid residues, and are not limited to a minimum length. Polypeptides, including the provided antibodies and antibody chains and other peptides, e.g., linkers and ROR1-binding peptides, may include amino acid residues including natural and/or non-natural amino acid residues. The terms also include post-expression modifications of the polypeptide, for example, glycosylation, sialylation, acetylation, phosphorylation, and the like. In some aspects, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification.

### 1. Exemplary Antibodies

In some embodiments, the antibody, e.g., the anti-ROR1 antibody, e.g., antigen-binding antibody fragment, contains a heavy and/or light chain variable (V_{H} or V_{L}) region sequence as described, or a sufficient antigen-binding portion thereof. In some embodiments, the antibody, e.g., the anti-ROR1 antibody, e.g., antigen-binding antibody fragment, contains a heavy chain variable region (V_{H}) sequence and/or a light chain variable region (V_{L}) sequence as described, or a sufficient antigen-binding portion thereof. In some embodiments, the antibody, e.g., the anti-ROR1 antibody, e.g., antigen-binding antibody fragment, is a single chain fragment, such as a single chain Fv (scFv) fragment. In some aspects, the scFv comprises a V_{H} region and a V_{L} region. In some embodiments, the antibody, e.g., the anti-ROR1 antibody, e.g., antigen-binding antibody fragment, is a single domain antibody (sdAb), such as an antibody that contains a V_{H} region only.

In some embodiments, the anti-ROR1 antibody, e.g., antigen-binding antibody fragment, contains a V_{H} region sequence or sufficient antigen-binding portion thereof that contains a heavy chain complementarity determining region 1 (CDR-H1), a heavy chain complementarity determining region 2 (CDR-H2) and/or a heavy chain complementarity determining region 3 (CDR-H3) as described. In some embodiments, the anti-ROR1 antibody, e.g., antigen-binding antibody fragment, contains a V_{H} region sequence or sufficient antigen-binding portion thereof that contains a CDR-H1, a CDR-H2 and a CDR-H3 as described. In some embodiments, the anti-ROR1 antibody, e.g., antigen-binding antibody fragment, contains a V_{L} region sequence or sufficient antigen-binding portion that contains a light chain complementarity determining region 1 (CDR-L1), a light chain complementarity determining region 2 (CDR-L2), and/or a light chain complementarity determining region 3 (CDR-L3) as described. In some embodiments, the anti-ROR1 antibody, e.g., antigen-binding antibody fragment, contains a V_{L} region sequence or sufficient antigen-binding portion that contains a CDR-L1, a CDR-L2 and a CDR-L3 as described.

In some embodiments, the anti-ROR1 antibody, e.g., antigen-binding antibody fragment, contains a V_{H} region sequence that contains a CDR-H1, a CDR-H2 and/or a CDR-H3 as described and contains a V_{L} region sequence that contains a CDR-L1, a CDR-L2 and/or a CDR-L3 as described. In some embodiments, the anti-ROR1 antibody, e.g., antigen-binding antibody fragment, contains a V_{H} region sequence that contains a CDR-H1, a CDR-H2 and a CDR-H3 as described and contains a V_{L} region sequence that contains a CDR-L1, a CDR-L2 and a CDR-L3 as described. Also among the provided antibodies and fragment thereof are those having sequences at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98%, or at or about 99% identical to such a sequence, e.g., any of the CDR-H1, CDR-H2, CDR-H3, CDR-L1, CDR-L2, CDR-L3, V_{H}, V_{L}, scFv sequences or other sequences of the antibodies of fragment thereof described herein. In some aspects, among the provided antibodies and fragment thereof are those having sequences at least at or about 85% sequence identity to any such sequences. In some aspects, among the provided antibodies and fragment thereof are those having sequences at least at or about 86% sequence identity to any such sequences. In some aspects, among the provided antibodies and fragment thereof are those having sequences at least at or about 87% sequence identity to any such sequences. In some aspects, among the provided antibodies and fragment thereof are those having sequences at least at or about 88% sequence identity to any such sequences. In some aspects, among the provided antibodies and fragment thereof are those having sequences at least at or about 89% sequence identity to any such sequences. In some aspects, among the provided antibodies and fragment thereof are those having sequences at least at or about 90% sequence identity to any such sequences. In some aspects, among the provided antibodies and fragment thereof are those having sequences at least at or about 91% sequence identity to any such sequences. In some aspects, among the provided antibodies and fragment thereof are those having sequences at least at or about 92% sequence identity to any such sequences. In some aspects, among the provided antibodies and fragment thereof are those having sequences at least at or about 93% sequence identity to any such sequences. In some aspects, among the provided antibodies and fragment thereof are those having sequences at least at or about 94% sequence identity to any such sequences. In some aspects, among the provided antibodies and fragment thereof are those having sequences at least at or about 95% sequence identity to any such sequences. In some aspects, among the provided antibodies and fragment thereof are those having sequences at least at or about 96% sequence identity to any such sequences. In some aspects, among the provided antibodies and fragment thereof are those having sequences at least at or about 97% sequence identity to any such sequences. In some aspects, among the provided antibodies and fragment thereof are those having sequences at least at or about 98% sequence identity to any such sequences. In some aspects, among the provided antibodies and fragment thereof are those having sequences at least at or about 99% sequence identity to any such sequences.

In some embodiments, the antibody is an sdAb comprising only a V_{H} region sequence or a sufficient antigen-binding portion thereof, such as any of the V_{H} sequences described herein (e.g., a CDR-H1, a CDR-H2, a CDR-H3 and/or a CDR-H4). In some embodiments, the antibodies or antigen-binding fragments include those that are single domain antibodies, containing a V_{H} region that, without pairing with a V_{L} region) and/or without any additional antibody domain or binding site, are capable of specifically binding to ROR1.

In some embodiments, the V_{H} region of an antibody or antigen-binding fragment thereof comprises a CDR-H1, a CDR-H2 and/or a CDR-H3 according to Kabat numbering. In some embodiments, the V_{H} region of an antibody or antigen-binding fragment thereof comprises a CDR-H1, a CDR-H2 and/or a CDR-H3 according to Chothia numbering. In some embodiments, the V_{H} region of an antibody or antigen-binding fragment thereof comprises a CDR-H1, a CDR-H2 and/or a CDR-H3 according to AbM numbering. In some embodiments, the V_{H} region of an antibody or antigen-binding fragment thereof comprises a CDR-H1, a CDR-H2 and/or a CDR-H3 according to IgBLAST numbering. In some embodiments, the V_{H} region of an antibody or antigen-binding fragment thereof comprises a CDR-H1, a CDR-H2 and/or a CDR-H3 according to Kabat, Chothia, AbM, IMGT or IgBLAST numbering, or other numbering schemes.

In some embodiments of the antibody, antigen-binding fragment, receptor, e.g., CAR, conjugates or binding molecules provided herein, has a heavy chain variable (V_{H}) region having the amino acid sequence set forth in SEQ ID NO: 112, 121, 103 or 130, or an amino acid sequence that has at least at or about 85%, 86%, 87%, 88%, 89%, 90%, 91%, 92%, 93%, 94%, 95%, 96%, 97%, 98%, or 99% sequence identity to the V_{H} region amino acid sequence set forth in SEQ ID NO: 112, 121, 103 or 130, or contains a heavy chain complementarity determining region 1 (CDR-H1), a heavy chain complementarity determining region 2 (CDR-H2) and/or a heavy chain complementarity determining region 3 (CDR-H3) present in such a V_{H} sequence, such as one that contains a CDR-H1, a CDR-H2, and a CDR-H3 present in such a V_{H} sequence. In some embodiments of the antibody, antigen-binding fragment, receptor, e.g., CAR, conjugates or binding molecules provided herein, has a V_{H} region having the amino acid sequence set forth in SEQ ID NO: 112 or 121, or an amino acid sequence that has at least at or about 85%, 86%, 87%, 88%, 89%, 90%, 91%, 92%, 93%, 94%, 95%, 96%, 97%, 98%, or 99% sequence identity to the V_{H} region amino acid sequence set forth in SEQ ID NO: 112 or 121, or contains a CDR-H1, a CDR-H2 and/or a CDR-H3 present in such a V_{H} sequence, such as one that contains a CDR-H1, a CDR-H2, and a CDR-H3 present in such a V_{H} sequence.

In some embodiments of the antibody, antigen-binding fragment, receptor, e.g., CAR, conjugates or binding molecules provided herein, has a V_{H} region having the amino acid sequence set forth in SEQ ID NO: 112, or an amino acid sequence that has at least at or about 85%, 86%, 87%, 88%, 89%, 90%, 91%, 92%, 93%, 94%, 95%, 96%, 97%, 98%, or 99% sequence identity to the V_{H} region amino acid sequence set forth in SEQ ID NO: 112, or contains a CDR-H1, a CDR-H2 and/or a CDR-H3 present in such a V_{H} sequence, such as one that contains a CDR-H1, a CDR-H2, and a CDR-H3 present in such a V_{H} sequence. In some embodiments, the antibody, e.g., antigen-binding fragment thereof, has a V_{H} region having the amino acid sequence set forth in SEQ ID NO: 121, or an amino acid sequence that has at least at or about 85%, 86%, 87%, 88%, 89%, 90%, 91%, 92%, 93%, 94%, 95%, 96%, 97%, 98%, or 99% sequence identity to the V_{H} region amino acid sequence set forth in SEQ ID NO: 121, or contains a CDR-H1, a CDR-H2 and/or a CDR-H3 present in such a V_{H} sequence, such as one that contains a CDR-H1, a CDR-H2, and a CDR-H3 present in such a V_{H} sequence. In some embodiments of the antibody, antigen-binding fragment, receptor, e.g., CAR, conjugates or binding molecules provided herein, has a V_{H} region having the amino acid sequence set forth in SEQ ID NO: 103, or an amino acid sequence that has at least at or about 85%, 86%, 87%, 88%, 89%, 90%, 91%, 92%, 93%, 94%, 95%, 96%, 97%, 98%, or 99% sequence identity to the V_{H} region amino acid sequence set forth in SEQ ID NO: 103, or contains a CDR-H1, a CDR-H2 and/or a CDR-H3 present in such a V_{H} sequence, such as one that contains a CDR-H1, a CDR-H2, and a CDR-H3 present in such a V_{H} sequence. In some embodiments, the antibody, e.g., antigen-binding fragment thereof, has a V_{H} region having the amino acid sequence set forth in SEQ ID NO: 130, or an amino acid sequence that has at least at or about 85%, 86%, 87%, 88%, 89%, 90%, 91%, 92%, 93%, 94%, 95%, 96%, 97%, 98%, or 99% sequence identity to the V_{H} region amino acid sequence set forth in SEQ ID NO: 130, or contains a CDR-H1, a CDR-H2 and/or a CDR-H3 present in such a V_{H} sequence, such as one that contains a CDR-H1, a CDR-H2, and a CDR-H3 present in such a V_{H} sequence.

In some of any of the provided embodiments, the V_{H} region comprises a CDR-H1 comprising the sequence set forth in SEQ ID NO: 67, 82 or 52. In some of any of the provided embodiments, the V_{H} region comprises a CDR-H2 comprising the sequence set forth in SEQ ID NO: 71, 86, 56 or 97. In some of any of the provided embodiments, the V_{H} region comprises a CDR-H3 comprising the sequence set forth in SEQ ID NO: 73, 88, 58 or 99.

In some of any of the provided embodiments, the V_{H} region comprises a CDR-H1 comprising the sequence set forth in SEQ ID NO: 65, 80 or 50. In some of any of the provided embodiments, the V_{H} region comprises a CDR-H2 comprising the sequence set forth in SEQ ID NO: 69, 84, 54 or 95. In some of any of the provided embodiments, the V_{H} region comprises a CDR-H3 comprising the sequence set forth in SEQ ID NO: 73, 88, 58 or 99.

In some of any of the provided embodiments, the V_{H} region comprises a CDR-H1 comprising the sequence set forth in SEQ ID NO: 66, 81 or 51. In some of any of the provided embodiments, the V_{H} region comprises a CDR-H2 comprising the sequence set forth in SEQ ID NO: 70, 85, 55 or 96. In some of any of the provided embodiments, the V_{H} region comprises a CDR-H3 comprising the sequence set forth in SEQ ID NO: 73, 88, 58 or 99.

In some of any of the provided embodiments, the V_{H} region comprises a CDR-H1 comprising the sequence set forth in SEQ ID NO: 68, 83 or 53. In some of any of the provided embodiments, the V_{H} region comprises a CDR-H2 comprising the sequence set forth in SEQ ID NO: 72, 87, 57 or 98. In some of any of the provided embodiments, the V_{H} region comprises a CDR-H3 comprising the sequence set forth in SEQ ID NO: 74, 89, 59 or 100.

In some embodiments of the antibody, antigen-binding fragment, receptor, e.g., CAR, conjugates or binding molecules provided herein, the V_{H} region comprises a CDR-H1 comprising the sequence set forth in SEQ ID NO: 67, 82 or 52, a CDR-H2 comprising the sequence set forth in SEQ ID NO: 71, 86, 56 or 97, and a CDR-H3 comprising the sequence set forth in SEQ ID NO: 73, 88, 58 or 99.

In some embodiments of the antibody, antigen-binding fragment, receptor, e.g., CAR, conjugates or binding molecules provided herein, the V_{H} region comprises a CDR-H1 comprising the sequence set forth in SEQ ID NO: 65, 80 or 50, a CDR-H2 comprising the sequence set forth in SEQ ID NO: 69, 84, 54 or 95, and a CDR-H3 comprising the sequence set forth in SEQ ID NO: 73, 88, 58 or 99. In some embodiments of the antibody, antigen-binding fragment, receptor, e.g., CAR, conjugates or binding molecules provided herein, the V_{H} region comprises a CDR-H1 comprising the sequence set forth in SEQ ID NO: 66, 81 or 51, a CDR-H2 comprising the sequence set forth in SEQ ID NO: 70, 85, 55 or 96, and a CDR-H3 comprising the sequence set forth in SEQ ID NO: 73, 88, 58 or 99. In some embodiments of the antibody, antigen-binding fragment, receptor, e.g., CAR, conjugates or binding molecules provided herein, the V_{H} region comprises a CDR-H1 comprising the sequence set forth in SEQ ID NO: 68, 83 or 53, a CDR-H2 comprising the sequence set forth in SEQ ID NO: 72, 87, 57 or 98, and a CDR-H3 comprising the sequence set forth in SEQ ID NO: 74, 89, 59 or 100.

In some embodiments of the antibody, antigen-binding fragment, receptor, e.g., CAR, conjugates or binding molecules provided herein, the V_{H} region comprises a CDR-H1 comprising the sequence set forth in SEQ ID NO: 67 or 82, a CDR-H2 comprising the sequence set forth in SEQ ID NO: 71 or 86, and a CDR-H3 comprising the sequence set forth in SEQ ID NO: 73 or 88. In some embodiments of the antibody, antigen-binding fragment, receptor, e.g., CAR, conjugates or binding molecules provided herein, the V_{H} region comprises a CDR-H1 comprising the sequence set forth in SEQ ID NO: 65 or 80, a CDR-H2 comprising the sequence set forth in SEQ ID NO: 69 or 84, and a CDR-H3 comprising the sequence set forth in SEQ ID NO: 73 or 88. In some embodiments of the antibody, antigen-binding fragment, receptor, e.g., CAR, conjugates or binding molecules provided herein, the V_{H} region comprises a CDR-H1 comprising the sequence set forth in SEQ ID NO: 66 or 81, a CDR-H2 comprising the sequence set forth in SEQ ID NO: 70 or 85, and a CDR-H3 comprising the sequence set forth in SEQ ID NO: 73 or 88. In some embodiments of the antibody, antigen-binding fragment, receptor, e.g., CAR, conjugates or binding molecules provided herein, the V_{H} region comprises a CDR-H1 comprising the sequence set forth in SEQ ID NO: 68 or 83, a CDR-H2 comprising the sequence set forth in SEQ ID NO: 72 or 87, and a CDR-H3 comprising the sequence set forth in SEQ ID NO: 74 or 89.

In some embodiments of the antibody, antigen-binding fragment, receptor, e.g., CAR, conjugates or binding molecules provided herein, the V_{H} region comprises a CDR-H1, a CDR-H2 and a CDR-H3 comprising the sequence set forth in SEQ ID NOS:67, 71 and 73, respectively; SEQ ID NOS:82, 86 and 88; SEQ ID NOS:52, 56 and 58, respectively; or SEQ ID NOS:52, 97 and 99, respectively. In some embodiments of the antibody, antigen-binding fragment, receptor, e.g., CAR, conjugates or binding molecules provided herein, the V_{H} region comprises a CDR-H1, a CDR-H2 and a CDR-H3 comprising the sequence set forth in SEQ ID NOS:65, 69 and 73, respectively; SEQ ID NOS:80, 84 and 88, respectively; SEQ ID NOS:50, 54 and 58, respectively; or SEQ ID NOS:50, 95 and 99, respectively. In some embodiments of the antibody, antigen-binding fragment, receptor, e.g., CAR, conjugates or binding molecules provided herein, the V_{H} region comprises a CDR-H1, a CDR-H2 and a CDR-H3 comprising the sequence set forth in SEQ ID NOS:66, 70 and 73, respectively; SEQ ID NOS:81, 85 and 88, respectively; SEQ ID NOS:51, 55 and 58, respectively; or SEQ ID NOS:51, 96 and 99. In some embodiments of the antibody, antigen-binding fragment, receptor, e.g., CAR, conjugates or binding molecules provided herein, the V_{H} region comprises a CDR-H1, a CDR-H2 and a CDR-H3 comprising the sequence set forth in SEQ ID NOS:68, 72 and 74, respectively; SEQ ID NOS:83, 87 and 89, respectively; SEQ ID NOS:53, 57 and 59, respectively; or SEQ ID NOS:53, 98 and 100, respectively.

In some embodiments of the antibody, antigen-binding fragment, receptor, e.g., CAR, conjugates or binding molecules provided herein, the V_{H} region comprises a CDR-H1, a CDR-H2 and a CDR-H3 comprising the sequence set forth in SEQ ID NOS:67, 71 and 73, respectively. In some embodiments of the antibody, antigen-binding fragment, receptor, e.g., CAR, conjugates or binding molecules provided herein, the V_{H} region comprises a CDR-H1, a CDR-H2 and a CDR-H3 comprising the sequence set forth in SEQ ID NOS:65, 69 and 73, respectively. In some embodiments of the antibody, antigen-binding fragment, receptor, e.g., CAR, conjugates or binding molecules provided herein, the V_{H} region comprises a CDR-H1, a CDR-H2 and a CDR-H3 comprising the sequence set forth in SEQ ID NOS:66, 70 and 73, respectively. In some embodiments of the antibody, antigen-binding fragment, receptor, e.g., CAR, conjugates or binding molecules provided herein, the V_{H} region comprises a CDR-H1, a CDR-H2 and a CDR-H3 comprising the sequence set forth in SEQ ID NOS:68, 72 and 74, respectively.

In some embodiments of the antibody, antigen-binding fragment, receptor, e.g., CAR, conjugates or binding molecules provided herein, the V_{H} region comprises a CDR-H1, a CDR-H2 and a CDR-H3 comprising the sequence set forth in SEQ ID NOS:82, 86 and 88, respectively. In some embodiments of the antibody, antigen-binding fragment, receptor, e.g., CAR, conjugates or binding molecules provided herein, the V_{H} region comprises a CDR-H1, a CDR-H2 and a CDR-H3 comprising the sequence set forth in SEQ ID NOS:80, 84 and 88, respectively. In some embodiments of the antibody, antigen-binding fragment, receptor, e.g., CAR, conjugates or binding molecules provided herein, the V_{H} region comprises a CDR-H1, a CDR-H2 and a CDR-H3 comprising the sequence set forth in SEQ ID NOS:81, 85 and 88, respectively. In some embodiments of the antibody, antigen-binding fragment, receptor, e.g., CAR, conjugates or binding molecules provided herein, the V_{H} region comprises a CDR-H1, a CDR-H2 and a CDR-H3 comprising the sequence set forth in SEQ ID NOS:83, 87 and 89, respectively.

In some embodiments of the antibody, antigen-binding fragment, receptor, e.g., CAR, conjugates or binding molecules provided herein, the V_{H} region comprises a CDR-H1, a CDR-H2 and a CDR-H3, respectively, comprising the amino acid sequence of a CDR-H1, a CDR-H2 and a CDR-H3 contained within the V_{H} region amino acid sequence set forth in any one of SEQ ID NOs: 112, 121, 103 or 130. In some embodiments of the antibody, antigen-binding fragment, receptor, e.g., CAR, conjugates or binding molecules provided herein, the V_{H} region comprises a CDR-H1, a CDR-H2 and a CDR-H3 contained within SEQ ID NO:112 or 121.

In some embodiments of the antibody, antigen-binding fragment, receptor, e.g., CAR, conjugates or binding molecules provided herein, the V_{H} region comprises a CDR-H1, a CDR-H2 and a CDR-H3 contained within SEQ ID NO:112. In some embodiments of the antibody, antigen-binding fragment, receptor, e.g., CAR, conjugates or binding molecules provided herein, the V_{H} region comprises a CDR-H1, a CDR-H2 and a CDR-H3 contained within SEQ ID NO:121. In some embodiments of the antibody, antigen-binding fragment, receptor, e.g., CAR, conjugates or binding molecules provided herein, the V_{H} region comprises a CDR-H1, a CDR-H2 and a CDR-H3 contained within SEQ ID NO:103. In some embodiments of the antibody, antigen-binding fragment, receptor, e.g., CAR, conjugates or binding molecules provided herein, the V_{H} region comprises a CDR-H1, a CDR-H2 and a CDR-H3 contained within SEQ ID NO:130.

In some embodiments of the antibody, antigen-binding fragment, receptor, e.g., CAR, conjugates or binding molecules provided herein, the V_{H} region comprises any of the CDR-H1, the CDR-H2 and the CDR-H3 as described and comprises a framework region 1 (FR1), a FR2, a FR3 and/or a FR4 having at least at or about 85%, 86%, 87%, 88%, 89%, 90%, 91%, 92%, 93%, 94%, 95%, 96%, 97%, 98% or 99%, respectively, to a FR1, a FR2, a FR3 and/or a FR4 contained within the V_{H} region amino acid sequence set forth in any one of SEQ ID NOs: 112, 121, 103 or 130. In some of any embodiments, the V_{H} region comprises a FR1, a FR2, a FR3 and/or a FR4 having at least at or about 85%, 86%, 87%, 88%, 89%, 90%, 91%, 92%, 93%, 94%, 95%, 96%, 97%, 98% or 99%, respectively, to a FR1, a FR2, a FR3 and/or a FR4 contained within the V_{H} region amino acid sequence set forth in SEQ ID NO: 112. In some of any embodiments, the V_{H} region comprises a FR1, a FR2, a FR3 and/or a FR4 having at least at or about 85%, 86%, 87%, 88%, 89%, 90%, 91%, 92%, 93%, 94%, 95%, 96%, 97%, 98% or 99%, respectively, to a FR1, a FR2, a FR3 and/or a FR4 contained within the V_{H} region amino acid sequence set forth in SEQ ID NO: 121. In some of any embodiments, the V_{H} region comprises a FR1, a FR2, a FR3 and/or a FR4 having at least at or about 85%, 86%, 87%, 88%, 89%, 90%, 91%, 92%, 93%, 94%, 95%, 96%, 97%, 98% or 99%, respectively, to a FR1, a FR2, a FR3 and/or a FR4 contained within the V_{H} region amino acid sequence set forth in SEQ ID NO: 103. In some of any embodiments, the V_{H} region comprises a FR1, a FR2, a FR3 and/or a FR4 having at least at or about 85%, 86%, 87%, 88%, 89%, 90%, 91%, 92%, 93%, 94%, 95%, 96%, 97%, 98% or 99%, respectively, to a FR1, a FR2, a FR3 and/or a FR4 contained within the V_{H} region amino acid sequence set forth in SEQ ID NO: 130.

In some embodiments of the antibody, antigen-binding fragment, receptor, e.g., CAR, conjugates or binding molecules provided herein, the V_{H} region is or comprises the amino acid sequence set forth in any one of SEQ ID NOs: 112, 121, 103 or 130. In some embodiments of the antibody, antigen-binding fragment, receptor, e.g., CAR, conjugates or binding molecules provided herein, the V_{H} region is or comprises the amino acid sequence set forth in SEQ ID NO:112. In some embodiments of the antibody, antigen-binding fragment, receptor, e.g., CAR, conjugates or binding molecules provided herein, the V_{H} region is or comprises the amino acid sequence set forth in SEQ ID NO:121. In some embodiments of the antibody, antigen-binding fragment, receptor, e.g., CAR, conjugates or binding molecules provided herein, the V_{H} region is or comprises the amino acid sequence set forth in SEQ ID NO:103. In some embodiments of the antibody, antigen-binding fragment, receptor, e.g., CAR, conjugates or binding molecules provided herein, the V_{H} region is or comprises the amino acid sequence set forth in SEQ ID NO:130.

In some embodiments of the antibody, antigen-binding fragment, receptor, e.g., CAR, conjugates or binding molecules provided herein, the V_{H} region is or comprises the amino acid sequence encoded by SEQ ID NO: 110, 119, 101 or 128 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 110, 119, 101 or 128. In some embodiments of the antibody, antigen-binding fragment, receptor, e.g., CAR, conjugates or binding molecules provided herein, the V_{H} region is or comprises the amino acid sequence encoded by SEQ ID NO: 111, 120, 102 or 129.

In some embodiments of the antibody, antigen-binding fragment, receptor, e.g., CAR, conjugates or binding molecules provided herein, the V_{H} region is or comprises the amino acid sequence encoded by SEQ ID NO: 110 or 119 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 110 or 119. In some embodiments of the antibody, antigen-binding fragment, receptor, e.g., CAR, conjugates or binding molecules provided herein, the V_{H} region is or comprises the amino acid sequence encoded by SEQ ID NO: 111 or 120.

In some embodiments of the antibody, antigen-binding fragment, receptor, e.g., CAR, conjugates or binding molecules provided herein, the V_{H} region is or comprises the amino acid sequence encoded by SEQ ID NO: 110 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 110. In some embodiments of the antibody, antigen-binding fragment, receptor, e.g., CAR, conjugates or binding molecules provided herein, the V_{H} region is or comprises the amino acid sequence encoded by SEQ ID NO: 111.

In some embodiments of the antibody, antigen-binding fragment, receptor, e.g., CAR, conjugates or binding molecules provided herein, the V_{H} region is or comprises the amino acid sequence encoded by SEQ ID NO: 119 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 119. In some embodiments of the antibody, antigen-binding fragment, receptor, e.g., CAR, conjugates or binding molecules provided herein, the V_{H} region is or comprises the amino acid sequence encoded by SEQ ID NO: 120.

In some embodiments of the antibody, antigen-binding fragment, receptor, e.g., CAR, conjugates or binding molecules provided herein, the V_{H} region is or comprises the amino acid sequence encoded by SEQ ID NO: 101 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 101. In some embodiments of the antibody, antigen-binding fragment, receptor, e.g., CAR, conjugates or binding molecules provided herein, the V_{H} region is or comprises the amino acid sequence encoded by SEQ ID NO: 102.

In some embodiments of the antibody, antigen-binding fragment, receptor, e.g., CAR, conjugates or binding molecules provided herein, the V_{H} region is or comprises the amino acid sequence encoded by SEQ ID NO: 128 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 128. In some embodiments of the antibody, antigen-binding fragment, receptor, e.g., CAR, conjugates or binding molecules provided herein, the V_{H} region is or comprises the amino acid sequence encoded by SEQ ID NO: 129. Also provided are polynucleotides that contain any of the nucleotide sequences described herein, e.g., encoding all of a portion of the provided binding molecules.

In some embodiments, the antibody or antibody fragment, in the provided CAR (e.g., an anti-ROR1 CAR), comprises a light chain or a sufficient antigen binding portion thereof. For example, in some embodiments, the antibody or antigen-binding fragment thereof contains a variable light chain (V_{L}) region, or a sufficient antigen-binding portion of a V_{L} region. In some embodiments, the antibody or antigen-binding fragment thereof contains a V_{H} region and a variable light chain (V_{L}) region, or a sufficient antigen-binding portion of a V_{H} and V_{L} region. In any such embodiments, a V_{H} region sequence can be any of the V_{H} region sequence described herein. In any such embodiments, a V_{L} region sequence can be any of the V_{L} region sequence described herein. In any such embodiments, any of the V_{H} region sequence and any of the V_{L} region sequence described herein can be used in combination. In some of any such embodiments, any one or more of the CDR-H1, the CDR-H2 and/or the CDR-H3 sequences described herein, and any one or more of the CDR-L1, the CDR-L2 and/or the CDR-L3 sequences described herein can be used in combination. In some such embodiments, the antibody is an antigen-binding fragment, such as a Fab or an scFv. In some embodiments, the antibody or antigen-binding fragment further comprises at least a portion of an immunoglobulin constant region or a variant thereof. In some such embodiments, the antibody is a full-length antibody that also contains a constant region.

In some embodiments, a binding molecule, such as a receptor, e.g., a CAR provided herein, contains an antibody such as an anti-ROR1 antibody, or antigen-binding fragment thereof that contains any of contains a V_{L} region or a sufficient antigen binding portion thereof. For example, in some embodiments, the CAR contains an antibody or antigen-binding fragment thereof that contains a V_{H} region and a V_{L} region, or a sufficient antigen-binding portion of a V_{H} and V_{L} region. In any such embodiments, a V_{H} region sequence can be any of the V_{H} region sequence described herein. In any such embodiments, a V_{L} region sequence can be any of the V_{L} region sequence described herein. In any such embodiments, any of the V_{H} region sequence and any of the V_{L} region sequence described herein can be used in combination. In some of any such embodiments, any one or more of the CDR-H1, the CDR-H2 and/or the CDR-H3 sequences described herein, and any one or more of the CDR-L1, the CDR-L2 and/or the CDR-L3 sequences described herein can be used in combination. In some such embodiments, the antibody contained in the provided recombinant receptor is an antigen-binding fragment, such as a Fab or an scFv. In some such embodiments, the receptor, e.g., CAR, further contains a spacer, such as a portion of an immunoglobulin constant region or a variant thereof, for example, as described below in Section I.B.

In some embodiments, the V_{H} region of an antibody or antigen-binding fragment thereof comprises a CDR-L1, a CDR-L2 and/or a CDR-L3 according to Kabat numbering. In some embodiments, the V_{H} region of an antibody or antigen-binding fragment thereof comprises a CDR-L1, a CDR-L2 and/or a CDR-L3 according to Chothia numbering. In some embodiments, the V_{H} region of an antibody or antigen-binding fragment thereof comprises a CDR-L1, a CDR-L2 and/or a CDR-L3 according to AbM numbering. In some embodiments, the V_{H} region of an antibody or antigen-binding fragment thereof comprises a CDR-L1, a CDR-L2 and/or a CDR-L3 according to IMGT numbering. In some embodiments, the V_{H} region of an antibody or antigen-binding fragment thereof comprises a CDR-L1, a CDR-L2 and/or a CDR-L3 according to IgBLAST numbering. In some embodiments, the V_{H} region of an antibody or antigen-binding fragment thereof comprises a CDR-L1, a CDR-L2 and/or a CDR-L3 according to Kabat, Chothia, AbM, IMGT or IgBLAST numbering, or other numbering schemes.

In some embodiments of the antibody, antigen-binding fragment, receptor, e.g., CAR, conjugates or binding molecules provided herein, has a light chain variable (V_{L}) region having the amino acid sequence set forth in SEQ ID NO: 115, 124 or 106, or an amino acid sequence that has at least at or about 85%, 86%, 87%, 88%, 89%, 90%, 91%, 92%, 93%, 94%, 95%, 96%, 97%, 98%, or 99% sequence identity to the V_{L} region amino acid sequence set forth in SEQ ID NO: 115, 124 or 106, or contains a light chain complementarity determining region 1 (CDR-L1), a light chain complementarity determining region 2 (CDR-L2) and/or a light chain complementarity determining region 3 (CDR-L3) present in such a V_{L} sequence, such as one that contains a CDR-L1, a CDR-L2, and a CDR-L3 present in such a V_{L} sequence. In some embodiments of the antibody, antigen-binding fragment, receptor, e.g., CAR, conjugates or binding molecules provided herein, has a V_{L} region having the amino acid sequence set forth in SEQ ID NO:115 or 124, or an amino acid sequence that has at least at or about 85%, 86%, 87%, 88%, 89%, 90%, 91%, 92%, 93%, 94%, 95%, 96%, 97%, 98%, or 99% sequence identity to the V_{L} region amino acid sequence set forth in SEQ ID NO: 115 or 124, or contains a CDR-L1, a CDR-L2 and/or a CDR-L3 present in such a V_{L} sequence, such as one that contains a CDR-L1, a CDR-L2, and a CDR-L3 present in such a V_{L} sequence.

In some embodiments of the antibody, antigen-binding fragment, receptor, e.g., CAR, conjugates or binding molecules provided herein, has a V_{L} region having the amino acid sequence set forth in SEQ ID NO: 115, or an amino acid sequence that has at least at or about 85%, 86%, 87%, 88%, 89%, 90%, 91%, 92%, 93%, 94%, 95%, 96%, 97%, 98%, or 99% sequence identity to the V_{L} region amino acid sequence set forth in SEQ ID NO: 115, or contains a CDR-L1, a CDR-L2 and/or a CDR-L3 present in such a V_{L} sequence, such as one that contains a CDR-L1, a CDR-L2, and a CDR-L3 present in such a V_{L} sequence. In some embodiments, the antibody, e.g., antigen-binding fragment thereof, has a V_{L} region having the amino acid sequence set forth in SEQ ID NO: 124, or an amino acid sequence that has at least at or about 85%, 86%, 87%, 88%, 89%, 90%, 91%, 92%, 93%, 94%, 95%, 96%, 97%, 98%, or 99% sequence identity to the V_{L} region amino acid sequence set forth in SEQ ID NO: 124, or contains a CDR-L1, a CDR-L2 and/or a CDR-L3 present in such a V_{L} sequence, such as one that contains a CDR-L1, a CDR-L2, and a CDR-L3 present in such a V_{L} sequence. In some embodiments, the antibody, e.g., antigen-binding fragment thereof, has a V_{L} region having the amino acid sequence set forth in SEQ ID NO: 106, or an amino acid sequence that has at least at or about 85%, 86%, 87%, 88%, 89%, 90%, 91%, 92%, 93%, 94%, 95%, 96%, 97%, 98%, or 99% sequence identity to the V_{L} region amino acid sequence set forth in SEQ ID NO: 106, or contains a CDR-L1, a CDR-L2 and/or a CDR-L3 present in such a V_{L} sequence, such as one that contains a CDR-L1, a CDR-L2, and a CDR-L3 present in such a V_{L} sequence.

In some of any of the provided embodiments, the V_{L} region comprises a CDR-L1 comprising the sequence set forth in SEQ ID NO: 75, 90 or 60. In some of any of the provided embodiments, the V_{L} region comprises a CDR-L2 comprising the sequence set forth in SEQ ID NO: 77, 92 or 62. In some of any of the provided embodiments, the V_{L} region comprises a CDR-L3 comprising the sequence set forth in SEQ ID NO: 79, 94 or 64.

In some of any of the provided embodiments, the V_{L} region comprises a CDR-L1 comprising the sequence set forth in SEQ ID NO: 76, 91 or 61. In some of any of the provided embodiments, the V_{L} region comprises a CDR-L2 comprising the sequence set forth in SEQ ID NO: 78, 93 or 63. In some of any of the provided embodiments, the V_{L} region comprises a CDR-L3 comprising the sequence set forth in SEQ ID NO: 79, 94 or 64.

In some embodiments of the antibody, antigen-binding fragment, receptor, e.g., CAR, conjugates or binding molecules provided herein, the V_{L} region comprises a CDR-L1 comprising the sequence set forth in SEQ ID NO: 75, 90 or 60, a CDR-L2 comprising the sequence set forth in SEQ ID NO: 77, 92 or 62, and a CDR-L3 comprising the sequence set forth in SEQ ID NO: 79, 94 or 64. In some embodiments of the antibody, antigen-binding fragment, receptor, e.g., CAR, conjugates or binding molecules provided herein, the V_{L} region comprises a CDR-L1 comprising the sequence set forth in SEQ ID NO: 76, 91 or 61, a CDR-L2 comprising the sequence set forth in SEQ ID NO: 78, 93 or 63; and a CDR-L3 comprising the sequence set forth in SEQ ID NO: 79, 94 or 64.

In some embodiments of the antibody, antigen-binding fragment, receptor, e.g., CAR, conjugates or binding molecules provided herein, the V_{L} region comprises a CDR-L1 comprising the sequence set forth in SEQ ID NO: 75 or 90, a CDR-L2 comprising the sequence set forth in SEQ ID NO: 77 or 92; and a CDR-L3 comprising the sequence set forth in SEQ ID NO: 79 or 94. In some embodiments of the antibody, antigen-binding fragment, receptor, e.g., CAR, conjugates or binding molecules provided herein, the V_{L} region comprises a CDR-L1 comprising the sequence set forth in SEQ ID NO: 76 or 91, a CDR-L2 comprising the sequence set forth in SEQ ID NO: 78 or 93; and a CDR-L3 comprising the sequence set forth in SEQ ID NO: 79 or 94.

In some embodiments of the antibody, antigen-binding fragment, receptor, e.g., CAR, conjugates or binding molecules provided herein, the V_{L} region comprises a CDR-L1, a CDR-L2 and a CDR-L3 comprising the sequence set forth in SEQ ID NOS:75, 77 and 79, respectively; SEQ ID NOS:90, 92 and 94; SEQ ID NOS:60, 62 and 64, respectively; or SEQ ID NOS:60, 62 and 64, respectively. In some embodiments of the antibody, antigen-binding fragment, receptor, e.g., CAR, conjugates or binding molecules provided herein, the V_{L} region comprises a CDR-L1, a CDR-L2 and a CDR-L3 comprising the sequence set forth in SEQ ID NOS:76, 78 and 79, respectively; SEQ ID NOS:91, 93 and 94, respectively; SEQ ID NOS:60, 63 and 64, respectively; or SEQ ID NOS:61, 63 and 64, respectively.

In some embodiments of the antibody, antigen-binding fragment, receptor, e.g., CAR, conjugates or binding molecules provided herein, the V_{L} region comprises a CDR-L1, a CDR-L2 and a CDR-L3 comprising the sequence set forth in SEQ ID NOS:75, 77 and 79, respectively. In some embodiments of the antibody, antigen-binding fragment, receptor, e.g., CAR, conjugates or binding molecules provided herein, the V_{L} region comprises a CDR-L1, a CDR-L2 and a CDR-L3 comprising the sequence set forth in SEQ ID NOS:76, 78 and 79, respectively.

In some embodiments of the antibody, antigen-binding fragment, receptor, e.g., CAR, conjugates or binding molecules provided herein, the V_{L} region comprises a CDR-L1, a CDR-L2 and a CDR-L3 comprising the sequence set forth in SEQ ID NOS:90, 92 and 94, respectively. In some embodiments of the antibody, antigen-binding fragment, receptor, e.g., CAR, conjugates or binding molecules provided herein, the V_{L} region comprises a CDR-L1, a CDR-L2 and a CDR-L3 comprising the sequence set forth in SEQ ID NOS:91, 93 and 94, respectively.

In some embodiments of the antibody, antigen-binding fragment, receptor, e.g., CAR, conjugates or binding molecules provided herein, the V_{L} region comprises a CDR-L1, a CDR-L2 and a CDR-L3, respectively, comprising the amino acid sequence of a CDR-L1, a CDR-L2 and a CDR-L3 contained within the V_{L} region amino acid sequence set forth in any one of SEQ ID NOs: 115, 124 or 106. In some embodiments of the antibody, antigen-binding fragment, receptor, e.g., CAR, conjugates or binding molecules provided herein, the V_{L} region comprises a CDR-L1, a CDR-L2 and a CDR-L3 contained within SEQ ID NO: 115 or 124.

In some embodiments of the antibody, antigen-binding fragment, receptor, e.g., CAR, conjugates or binding molecules provided herein, the V_{L} region comprises CDR-L1, a CDR-L2 and a CDR-L3 contained within SEQ ID NO: 115. In some embodiments of the antibody, antigen-binding fragment, receptor, e.g., CAR, conjugates or binding molecules provided herein, the V_{L} region comprises CDR-L1, a CDR-L2 and a CDR-L3 contained within SEQ ID NO: 124. In some embodiments of the antibody, antigen-binding fragment, receptor, e.g., CAR, conjugates or binding molecules provided herein, the V_{L} region comprises CDR-L1, a CDR-L2 and a CDR-L3 contained within SEQ ID NO: 106.

In some embodiments of the antibody, antigen-binding fragment, receptor, e.g., CAR, conjugates or binding molecules provided herein, the V_{L} region comprises any of the CDR-L1, the CDR-L2 and the CDR-L3 as described and comprises a framework region 1 (FR1), a FR2, a FR3 and/or a FR4 having at least at or about 85%, 86%, 87%, 88%, 89%, 90%, 91%, 92%, 93%, 94%, 95%, 96%, 97%, 98% or 99%, respectively, to a FR1, a FR2, a FR3 and/or a FR4 contained within the V_{L} region amino acid sequence set forth in any one of SEQ ID NOs: 115, 124 or 106. In some of any embodiments, the V_{L} region comprises a FR1, a FR2, a FR3 and/or a FR4 having at least at or about 85%, 86%, 87%, 88%, 89%, 90%, 91%, 92%, 93%, 94%, 95%, 96%, 97%, 98% or 99%, respectively, to a FR1, a FR2, a FR3 and/or a FR4 contained within the V_{L} region amino acid sequence set forth in SEQ ID NO: 115. In some of any embodiments, the V_{L} region comprises a FR1, a FR2, a FR3 and/or a FR4 having at least at or about 85%, 86%, 87%, 88%, 89%, 90%, 91%, 92%, 93%, 94%, 95%, 96%, 97%, 98% or 99%, respectively, to a FR1, a FR2, a FR3 and/or a FR4 contained within the V_{L} region amino acid sequence set forth in SEQ ID NO: 124. In some of any embodiments, the V_{L} region comprises a FR1, a FR2, a FR3 and/or a FR4 having at least at or about 85%, 86%, 87%, 88%, 89%, 90%, 91%, 92%, 93%, 94%, 95%, 96%, 97%, 98% or 99%, respectively, to a FR1, a FR2, a FR3 and/or a FR4 contained within the V_{L} region amino acid sequence set forth in SEQ ID NO: 106.

In some embodiments of the antibody, antigen-binding fragment, receptor, e.g., CAR, conjugates or binding molecules provided herein, the V_{L} region is or comprises the amino acid sequence set forth in any one of SEQ ID NOs: 115, 124 or 106. In some embodiments of the antibody, antigen-binding fragment, receptor, e.g., CAR, conjugates or binding molecules provided herein, the V_{L} region is or comprises the amino acid sequence set forth in SEQ ID NO: 115. In some embodiments of the antibody, antigen-binding fragment, receptor, e.g., CAR, conjugates or binding molecules provided herein, the V_{L} region is or comprises the amino acid sequence set forth in SEQ ID NO: 124. In some embodiments of the antibody, antigen-binding fragment, receptor, e.g., CAR, conjugates or binding molecules provided herein, the V_{L} region is or comprises the amino acid sequence set forth in SEQ ID NO: 106.

In some embodiments of the antibody, antigen-binding fragment, receptor, e.g., CAR, conjugates or binding molecules provided herein, the V_{L} region is or comprises the amino acid sequence encoded by SEQ ID NO: 113, 122 or 104, or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 113, 122 or 104. In some embodiments of the antibody, antigen-binding fragment, receptor, e.g., CAR, conjugates or binding molecules provided herein, the V_{L} region is or comprises the amino acid sequence encoded by SEQ ID NO: 114, 123, 105 or 131.

In some embodiments of the antibody, antigen-binding fragment, receptor, e.g., CAR, conjugates or binding molecules provided herein, the V_{L} region is or comprises the amino acid sequence encoded by SEQ ID NO: 113 or 122, or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 113 or 122. In some embodiments of the antibody, antigen-binding fragment, receptor, e.g., CAR, conjugates or binding molecules provided herein, the V_{L} region is or comprises the amino acid sequence encoded by SEQ ID NO: 114 or 123.

In some embodiments of the antibody, antigen-binding fragment, receptor, e.g., CAR, conjugates or binding molecules provided herein, the V_{L} region is or comprises the amino acid sequence encoded by SEQ ID NO: 113, or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 113. In some embodiments of the antibody, antigen-binding fragment, receptor, e.g., CAR, conjugates or binding molecules provided herein, the V_{L} region is or comprises the amino acid sequence encoded by SEQ ID NO: 114.

In some embodiments of the antibody, antigen-binding fragment, receptor, e.g., CAR, conjugates or binding molecules provided herein, the V_{L} region is or comprises the amino acid sequence encoded by SEQ ID NO: 122, or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 122. In some embodiments of the antibody, antigen-binding fragment, receptor, e.g., CAR, conjugates or binding molecules provided herein, the V_{L} region is or comprises the amino acid sequence encoded by SEQ ID NO: 123.

In some embodiments of the antibody, antigen-binding fragment, receptor, e.g., CAR, conjugates or binding molecules provided herein, the V_{L} region is or comprises the amino acid sequence encoded by SEQ ID NO: 104, or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 104. In some embodiments of the antibody, antigen-binding fragment, receptor, e.g., CAR, conjugates or binding molecules provided herein, the V_{L} region is or comprises the amino acid sequence encoded by SEQ ID NO: 105. In some embodiments of the antibody, antigen-binding fragment, receptor, e.g., CAR, conjugates or binding molecules provided herein, the V_{L} region is or comprises the amino acid sequence encoded by SEQ ID NO: 131.

In some embodiments, the antibody or antigen binding fragment is an scFv comprising a heavy chain variable (V_{H}) region and a light chain variable (V_{L}) region. In some embodiments of the antibody, antigen-binding fragment, receptor, e.g., CAR, conjugates or binding molecules provided herein, the V_{H} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 112, 121, 103 or 130, and the V_{L} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 115, 124 or 106. In some embodiments of the antibody, antigen-binding fragment, receptor, e.g., CAR, conjugates or binding molecules provided herein, the V_{H} region is or comprises an amino acid sequence having at least at or about 90%, 91%, 92%, 93%, 94%, 95%, 96%, 97%, 98% or 99% sequence identity to SEQ ID NO: 112 or 121, and the V_{L} region is or comprises an amino acid sequence having at least at or about 90%, 91%, 92%, 93%, 94%, 95%, 96%, 97%, 98%, or 99% sequence identity to SEQ ID NO:115 or 124.

In some embodiments of the antibody, antigen-binding fragment, receptor, e.g., CAR, conjugates or binding molecules provided herein, the V_{H} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO:112, and the V_{L} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 115; the V_{H} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO:121, and the V_{L} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO:124; the V_{H} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO:103, and the V_{L} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO:106; or the V_{H} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO:130, and the V_{L} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO:106.

In some embodiments of the antibody, antigen-binding fragment, receptor, e.g., CAR, conjugates or binding molecules provided herein, the V_{H} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO:112, and the V_{L} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 115. In some embodiments of the antibody, antigen-binding fragment, receptor, e.g., CAR, conjugates or binding molecules provided herein, the V_{H} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO:121, and the V_{L} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO:124. In some embodiments of the antibody, antigen-binding fragment, receptor, e.g., CAR, conjugates or binding molecules provided herein, the V_{H} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO:103, and the V_{L} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO:106. In some embodiments of the antibody, antigen-binding fragment, receptor, e.g., CAR, conjugates or binding molecules provided herein, the V_{H} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO:130, and the V_{L} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO:106.

In some embodiments of the antibody, antigen-binding fragment, receptor, e.g., CAR, conjugates or binding molecules provided herein, the V_{H} region of the antibody or antigen-binding fragment thereof comprises a CDR-H1, a CDR-H2, a CDR-H3, respectively, comprising the amino acid sequences of CDR-H1, a CDR-H2 and a CDR-H3 contained within the V_{H} region amino acid sequence selected from any one of SEQ ID NOs: 112, 121, 103 or 130; and comprises a CDR-L1, a CDR-L2, a CDR-L3, respectively, comprising the amino acid sequences of CDR-L1, a CDR-L2 and a CDR-L3, respectively contained within the V_{L} region amino acid sequence selected from any one of SEQ ID NOs: 115, 124 or 106. In some of any such embodiments, the V_{H} region comprises a CDR-H1, a CDR-H2 and a CDR-H3 contained within SEQ ID NO:112 or 121, and the V_{L} region comprises a CDR-L1, a CDR-L2 and a CDR-L3 contained within SEQ ID NO:115 or 124.

In some of any such embodiments, the V_{H} region comprises a CDR-H1, a CDR-H2 and a CDR-H3 contained within SEQ ID NO:112, and the V_{L} region comprises a CDR-L1, a CDR-L2 and a CDR-L3 contained within SEQ ID NO:115. In some of any such embodiments, the V_{H} region comprises a CDR-H1, a CDR-H2 and a CDR-H3 contained within SEQ ID NO:121, and the V_{L} region comprises a CDR-L1, a CDR-L2 and a CDR-L3 contained within SEQ ID: NO 124. In some of any such embodiments, the V_{H} region comprises a CDR-H1, a CDR-H2 and a CDR-H3 contained within SEQ ID NO:103, and the V_{L} region comprises a CDR-L1, a CDR-L2 and a CDR-L3 contained within SEQ ID: NO 106. In some of any such embodiments, the V_{H} region comprises a CDR-H1, a CDR-H2 and a CDR-H3 contained within SEQ ID NO:130, and the V_{L} region comprises a CDR-L1, a CDR-L2 and a CDR-L3 contained within SEQ ID: NO 106.

In some embodiments of the antibody, antigen-binding fragment, receptor, e.g., CAR, conjugates or binding molecules provided herein, the V_{H} region comprises a heavy chain complementarity determining region 1 (CDR-H1) comprising the sequence set forth in SEQ ID NO: 67, 82 or 52, a heavy chain complementarity determining region 2 (CDR-H2) comprising the sequence set forth in SEQ ID NO: 71, 86, 56 or 97, and a heavy chain complementarity determining region 3 (CDR-H3) comprising the sequence set forth in SEQ ID NO: 73, 88, 58 or 99, and the V_{L} region comprises a light chain complementarity determining region 1 (CDR-L1) comprising the sequence set forth in SEQ ID NO: 75, 90 or 60, a light chain complementarity determining region 2 (CDR-L2) comprising the sequence set forth in SEQ ID NO: 77, 92 or 62; and a light chain complementarity determining region 3 (CDR-L3) comprising the sequence set forth in SEQ ID NO: 79, 94 or 64.

In some embodiments of the antibody, antigen-binding fragment, receptor, e.g., CAR, conjugates or binding molecules provided herein, the V_{H} region comprises a CDR-H1 comprising the sequence set forth in SEQ ID NO: 65, 80 or 50, a CDR-H2 comprising the sequence set forth in SEQ ID NO: 69, 84, 54 or 95, and a CDR-H3 comprising the sequence set forth in SEQ ID NO: 73, 88, 58 or 99, and the V_{L} region comprises a CDR-L1 comprising the sequence set forth in SEQ ID NO: 75, 90 or 60, a CDR-L2 comprising the sequence set forth in SEQ ID NO: 77, 92 or 62; and a CDR-L3 comprising the sequence set forth in SEQ ID NO: 79, 94 or 64; or

In some embodiments of the antibody, antigen-binding fragment, receptor, e.g., CAR, conjugates or binding molecules provided herein, the V_{H} region comprises a CDR-H1 comprising the sequence set forth in SEQ ID NO: 66, 81 or 51, a CDR-H2 comprising the sequence set forth in SEQ ID NO: 70, 85, 55 or 96, and a CDR-H3 comprising the sequence set forth in SEQ ID NO: 73, 88, 58 or 99, and the V_{L} region comprises a CDR-L1 comprising the sequence set forth in SEQ ID NO: 75, 90 or 60, a CDR-L2 comprising the sequence set forth in SEQ ID NO: 77, 92 or 62; and a CDR-L3 comprising the sequence set forth in SEQ ID NO: 79, 94 or 64; or

In some embodiments of the antibody, antigen-binding fragment, receptor, e.g., CAR, conjugates or binding molecules provided herein, the V_{H} region comprises a CDR-H1 comprising the sequence set forth in SEQ ID NO: 68, 83 or 53, a CDR-H2 comprising the sequence set forth in SEQ ID NO: 72, 87, 57 or 98, and a CDR-H3 comprising the sequence set forth in SEQ ID NO: 74, 89, 59 or 100, and the V_{L} region comprises a CDR-L1 comprising the sequence set forth in SEQ ID NO: 76, 91 or 61, a CDR-L2 comprising the sequence set forth in SEQ ID NO: 78, 93 or 63; and a CDR-L3 comprising the sequence set forth in SEQ ID NO: 79, 94 or 64.

In some embodiments of the antibody, antigen-binding fragment, receptor, e.g., CAR, conjugates or binding molecules provided herein, the V_{H} region comprises a CDR-H1 comprising the sequence set forth in SEQ ID NO: 67 or 82, a CDR-H2 comprising the sequence set forth in SEQ ID NO: 71 or 86, and a CDR-H3 comprising the sequence set forth in SEQ ID NO: 73 or 88, and the V_{L} region comprises a CDR-L1 comprising the sequence set forth in SEQ ID NO: 75 or 90, a CDR-L2 comprising the sequence set forth in SEQ ID NO: 77 or 92; and a CDR-L3 comprising the sequence set forth in SEQ ID NO: 79 or 94.

In some embodiments of the antibody, antigen-binding fragment, receptor, e.g., CAR, conjugates or binding molecules provided herein, the V_{H} region comprises a CDR-H1 comprising the sequence set forth in SEQ ID NO: 65 or 80, a CDR-H2 comprising the sequence set forth in SEQ ID NO: 69 or 84, and a CDR-H3 comprising the sequence set forth in SEQ ID NO: 73 or 88, and the V_{L} region comprises a CDR-L1 comprising the sequence set forth in SEQ ID NO: 75 or 90, a CDR-L2 comprising the sequence set forth in SEQ ID NO: 77 or 92; and a CDR-L3 comprising the sequence set forth in SEQ ID NO: 79 or 94.

In some embodiments of the antibody, antigen-binding fragment, receptor, e.g., CAR, conjugates or binding molecules provided herein, the V_{H} region comprises a CDR-H1 comprising the sequence set forth in SEQ ID NO: 66 or 81, a CDR-H2 comprising the sequence set forth in SEQ ID NO: 70 or 85, and a CDR-H3 comprising the sequence set forth in SEQ ID NO: 73 or 88, and the V_{L} region comprises a CDR-L1 comprising the sequence set forth in SEQ ID NO: 75 or 90, a CDR-L2 comprising the sequence set forth in SEQ ID NO: 77 or 92; and a CDR-L3 comprising the sequence set forth in SEQ ID NO: 79 or 94.

In some embodiments of the antibody, antigen-binding fragment, receptor, e.g., CAR, conjugates or binding molecules provided herein, the V_{H} region comprises a CDR-H1 comprising the sequence set forth in SEQ ID NO: 68 or 83, a CDR-H2 comprising the sequence set forth in SEQ ID NO: 72 or 87, and a CDR-H3 comprising the sequence set forth in SEQ ID NO: 74 or 89, and the V_{L} region comprises a CDR-L1 comprising the sequence set forth in SEQ ID NO: 76 or 91, a CDR-L2 comprising the sequence set forth in SEQ ID NO: 78 or 93; and a CDR-L3 comprising the sequence set forth in SEQ ID NO: 79 or 94.

In some embodiments of the antibody, antigen-binding fragment, receptor, e.g., CAR, conjugates or binding molecules provided herein, the V_{H} region comprises a heavy chain complementarity determining region 1 (CDR-H1), a heavy chain complementarity determining region 2 (CDR-H2) and a heavy chain complementarity determining region 3 (CDR-H3) comprising the sequence set forth in SEQ ID NOS:67, 71 and 73, respectively, and the V_{L} region comprises a light chain complementarity determining region 1 (CDR-L1), a light chain complementarity determining region 2 (CDR-L2) and a light chain complementarity determining region 3 (CDR-L3) comprising the sequence set forth in SEQ ID NOS:75, 77 and 79, respectively; the V_{H} region comprises a CDR-H1, a CDR-H2 and a CDR-H3 comprising the sequence set forth in SEQ ID NOS:82, 86 and 88, respectively, and the V_{L} region comprises a CDR-L1, a CDR-L2 and a CDR-L3 comprising the sequence set forth in SEQ ID NOS:90, 92 and 94, respectively; the V_{H} region comprises a CDR-H1, a CDR-H2 and a CDR-H3 comprising the sequence set forth in SEQ ID NOS:52, 56 and 58, respectively, and the V_{L} region comprises a CDR-L1, a CDR-L2 and a CDR-L3 comprising the sequence set forth in SEQ ID NOS:60, 62 and 64, respectively; or the V_{H} region comprises a CDR-H1, a CDR-H2 and a CDR-H3 comprising the sequence set forth in SEQ ID NOS:52, 97 and 99, respectively, and the V_{L} region comprises a CDR-L1, a CDR-L2 and a CDR-L3 comprising the sequence set forth in SEQ ID NOS:60, 62 and 64, respectively.

In some embodiments of the antibody, antigen-binding fragment, receptor, e.g., CAR, conjugates or binding molecules provided herein, the V_{H} region comprises a CDR-H1, a CDR-H2 and a CDR-H3 comprising the sequence set forth in SEQ ID NOS:65, 69 and 73, respectively, and the V_{L} region comprises a CDR-L1, a CDR-L2 and a CDR-L3 comprising the sequence set forth in SEQ ID NOS:75, 77 and 79, respectively; the V_{H} region comprises a CDR-H1, a CDR-H2 and a CDR-H3 comprising the sequence set forth in SEQ ID NOS:80, 84 and 88, respectively, and the V_{L} region comprises a CDR-L1, a CDR-L2 and a CDR-L3 comprising the sequence set forth in SEQ ID NOS:90, 92 and 94, respectively; the V_{H} region comprises a CDR-H1, a CDR-H2 and a CDR-H3 comprising the sequence set forth in SEQ ID NOS:50, 54 and 58, respectively, and the V_{L} region comprises a CDR-L1, a CDR-L2 and a CDR-L3 comprising the sequence set forth in SEQ ID NOS:60, 62 and 64, respectively; or the V_{H} region comprises a CDR-H1, a CDR-H2 and a CDR-H3 comprising the sequence set forth in SEQ ID NOS:50, 95 and 99, respectively, and the V_{L} region comprises a CDR-L1, a CDR-L2 and a CDR-L3 comprising the sequence set forth in SEQ ID NOS:60, 62 and 64, respectively;

In some embodiments of the antibody, antigen-binding fragment, receptor, e.g., CAR, conjugates or binding molecules provided herein, the V_{H} region comprises a CDR-H1, a CDR-H2 and a CDR-H3 comprising the sequence set forth in SEQ ID NOS:66, 70 and 73, respectively, and the V_{L} region comprises a CDR-L1, a CDR-L2 and a CDR-L3 comprising the sequence set forth in SEQ ID NOS:75, 77 and 79, respectively; the V_{H} region comprises a CDR-H1, a CDR-H2 and a CDR-H3 comprising the sequence set forth in SEQ ID NOS:81, 85 and 88, respectively, and the V_{L} region comprises a CDR-L1, a CDR-L2 and a CDR-L3 comprising the sequence set forth in SEQ ID NOS:90, 92 and 94, respectively; the V_{H} region comprises a CDR-H1, a CDR-H2 and a CDR-H3 comprising the sequence set forth in SEQ ID NOS:51, 55 and 58, respectively, and the V_{L} region comprises a CDR-L1, a CDR-L2 and a CDR-L3 comprising the sequence set forth in SEQ ID NOS:60, 62 and 64, respectively; or the V_{H} region comprises a CDR-H1, a CDR-H2 and a CDR-H3 comprising the sequence set forth in SEQ ID NOS:51, 96 and 99, respectively, and the V_{L} region comprises a CDR-L1, a CDR-L2 and a CDR-L3 comprising the sequence set forth in SEQ ID NOS:60, 62 and 64, respectively;

In some embodiments of the antibody, antigen-binding fragment, receptor, e.g., CAR, conjugates or binding molecules provided herein, the V_{H} region comprises a CDR-H1, a CDR-H2 and a CDR-H3 comprising the sequence set forth in SEQ ID NOS:68, 72 and 74, respectively, and the V_{L} region comprises a CDR-L1, a CDR-L2 and a CDR-L3 comprising the sequence set forth in SEQ ID NOS:76, 78 and 79, respectively; or the V_{H} region comprises a CDR-H1, a CDR-H2 and a CDR-H3 comprising the sequence set forth in SEQ ID NOS:83, 87 and 89, respectively, and the V_{L} region comprises a CDR-L1, a CDR-L2 and a CDR-L3 comprising the sequence set forth in SEQ ID NOS:91, 93 and 94, respectively; the V_{H} region comprises a CDR-H1, a CDR-H2 and a CDR-H3 comprising the sequence set forth in SEQ ID NOS:53, 57 and 59, respectively, and the V_{L} region comprises a CDR-L1, a CDR-L2 and a CDR-L3 comprising the sequence set forth in SEQ ID NOS:60, 63 and 64, respectively; or the V_{H} region comprises a CDR-H1, a CDR-H2 and a CDR-H3 comprising the sequence set forth in SEQ ID NOS:53, 98 and 100, respectively, and the V_{L} region comprises a CDR-L1, a CDR-L2 and a CDR-L3 comprising the sequence set forth in SEQ ID NOS:61, 63 and 64, respectively.

In some embodiments of the antibody, antigen-binding fragment, receptor, e.g., CAR, conjugates or binding molecules provided herein, the V_{H} region comprises a CDR-H1, a CDR-H2 and a CDR-H3 comprising the sequence set forth in SEQ ID NOS:67, 71 and 73, respectively, and the V_{L} region comprises a CDR-L1, a CDR-L2 and a CDR-L3 comprising the sequence set forth in SEQ ID NOS:75, 77 and 79, respectively.

In some embodiments of the antibody, antigen-binding fragment, receptor, e.g., CAR, conjugates or binding molecules provided herein, the V_{H} region comprises a CDR-H1, a CDR-H2 and a CDR-H3 comprising the sequence set forth in SEQ ID NOS:65, 69 and 73, respectively, and the V_{L} region comprises a CDR-L1, a CDR-L2 and a CDR-L3 comprising the sequence set forth in SEQ ID NOS:75, 77 and 79, respectively.

In some embodiments of the antibody, antigen-binding fragment, receptor, e.g., CAR, conjugates or binding molecules provided herein, the V_{H} region comprises a CDR-H1, a CDR-H2 and a CDR-H3 comprising the sequence set forth in SEQ ID NOS:66, 70 and 73, respectively, and the V_{L} region comprises a CDR-L1, a CDR-L2 and a CDR-L3 comprising the sequence set forth in SEQ ID NOS:75, 77 and 79, respectively.

In some embodiments of the antibody, antigen-binding fragment, receptor, e.g., CAR, conjugates or binding molecules provided herein, the V_{H} region comprises a CDR-H1, a CDR-H2 and a CDR-H3 comprising the sequence set forth in SEQ ID NOS:68, 72 and 74, respectively, and the V_{L} region comprises a CDR-L1, a CDR-L2 and a CDR-L3 comprising the sequence set forth in SEQ ID NOS:76, 78 and 79, respectively.

In some embodiments of the antibody, antigen-binding fragment, receptor, e.g., CAR, conjugates or binding molecules provided herein, the V_{H} region comprises a CDR-H1, a CDR-H2 and a CDR-H3 comprising the sequence set forth in SEQ ID NOS:82, 86 and 88, respectively, and the V_{L} region comprises a CDR-L1, a CDR-L2 and a CDR-L3 comprising the sequence set forth in SEQ ID NOS:90, 92 and 94, respectively.

In some embodiments of the antibody, antigen-binding fragment, receptor, e.g., CAR, conjugates or binding molecules provided herein, the V_{H} region comprises a CDR-H1, a CDR-H2 and a CDR-H3 comprising the sequence set forth in SEQ ID NOS:80, 84 and 88, respectively, and the V_{L} region comprises a CDR-L1, a CDR-L2 and a CDR-L3 comprising the sequence set forth in SEQ ID NOS:90, 92 and 94, respectively.

In some embodiments of the antibody, antigen-binding fragment, receptor, e.g., CAR, conjugates or binding molecules provided herein, the V_{H} region comprises a CDR-H1, a CDR-H2 and a CDR-H3 comprising the sequence set forth in SEQ ID NOS:81, 85 and 88, respectively, and the V_{L} region comprises a CDR-L1, a CDR-L2 and a CDR-L3 comprising the sequence set forth in SEQ ID NOS:90, 92 and 94, respectively.

In some embodiments of the antibody, antigen-binding fragment, receptor, e.g., CAR, conjugates or binding molecules provided herein, the V_{H} region comprises a CDR-H1, a CDR-H2 and a CDR-H3 comprising the sequence set forth in SEQ ID NOS:83, 87 and 89, respectively, and the V_{L} region comprises a CDR-L1, a CDR-L2 and a CDR-L3 comprising the sequence set forth in SEQ ID NOS:91, 93 and 94, respectively.

In some embodiments of the antibody, antigen-binding fragment, receptor, e.g., CAR, conjugates or binding molecules provided herein, the V_{H} region comprises a CDR-H1, a CDR-H2 and a CDR-H3 comprising the sequence set forth in SEQ ID NOS:52, 56 and 58, respectively, and the V_{L} region comprises a CDR-L1, a CDR-L2 and a CDR-L3 comprising the sequence set forth in SEQ ID NOS:60, 62 and 64, respectively.

In some embodiments of the antibody, antigen-binding fragment, receptor, e.g., CAR, conjugates or binding molecules provided herein, the V_{H} region comprises a CDR-H1, a CDR-H2 and a CDR-H3 comprising the sequence set forth in SEQ ID NOS:50, 54 and 58, respectively, and the V_{L} region comprises a CDR-L1, a CDR-L2 and a CDR-L3 comprising the sequence set forth in SEQ ID NOS:60, 62 and 64, respectively.

In some embodiments of the antibody, antigen-binding fragment, receptor, e.g., CAR, conjugates or binding molecules provided herein, the V_{H} region comprises a CDR-H1, a CDR-H2 and a CDR-H3 comprising the sequence set forth in SEQ ID NOS:51, 55 and 58, respectively, and the V_{L} region comprises a CDR-L1, a CDR-L2 and a CDR-L3 comprising the sequence set forth in SEQ ID NOS:60, 62 and 64, respectively.

In some embodiments of the antibody, antigen-binding fragment, receptor, e.g., CAR, conjugates or binding molecules provided herein, the V_{H} region comprises a CDR-H1, a CDR-H2 and a CDR-H3 comprising the sequence set forth in SEQ ID NOS:53, 57 and 59, respectively, and the V_{L} region comprises a CDR-L1, a CDR-L2 and a CDR-L3 comprising the sequence set forth in SEQ ID NOS:60, 63 and 64, respectively.

In some embodiments of the antibody, antigen-binding fragment, receptor, e.g., CAR, conjugates or binding molecules provided herein, the V_{H} region comprises a CDR-H1, a CDR-H2 and a CDR-H3 comprising the sequence set forth in SEQ ID NOS:52, 97 and 99, respectively, and the V_{L} region comprises a CDR-L1, a CDR-L2 and a CDR-L3 comprising the sequence set forth in SEQ ID NOS:60, 62 and 64, respectively.

In some embodiments of the antibody, antigen-binding fragment, receptor, e.g., CAR, conjugates or binding molecules provided herein, the V_{H} region comprises a CDR-H1, a CDR-H2 and a CDR-H3 comprising the sequence set forth in SEQ ID NOS:50, 95 and 99, respectively, and the V_{L} region comprises a CDR-L1, a CDR-L2 and a CDR-L3 comprising the sequence set forth in SEQ ID NOS:60, 62 and 64, respectively.

In some embodiments of the antibody, antigen-binding fragment, receptor, e.g., CAR, conjugates or binding molecules provided herein, the V_{H} region comprises a CDR-H1, a CDR-H2 and a CDR-H3 comprising the sequence set forth in SEQ ID NOS:51, 96 and 99, respectively, and the V_{L} region comprises a CDR-L1, a CDR-L2 and a CDR-L3 comprising the sequence set forth in SEQ ID NOS:60, 62 and 64, respectively.

In some embodiments of the antibody, antigen-binding fragment, receptor, e.g., CAR, conjugates or binding molecules provided herein, the V_{H} region comprises a CDR-H1, a CDR-H2 and a CDR-H3 comprising the sequence set forth in SEQ ID NOS:53, 98 and 100, respectively, and the V_{L} region comprises a CDR-L1, a CDR-L2 and a CDR-L3 comprising the sequence set forth in SEQ ID NOS:61, 63 and 64, respectively.

In some embodiments, the V_{H} region of the antibody or antigen-binding fragment thereof, receptor, e.g., CAR, conjugates or binding molecules comprise the amino acid sequence of SEQ ID NOs: 112, 121, 103 or 130 and the V_{L} regions of the antibody or antigen-binding fragment comprises the amino acid sequence 115, 124 or 106. In some embodiments, the V_{H} and V_{L} regions of the antibody or antigen-binding fragment thereof, receptor, e.g., CAR, conjugates or binding molecules comprise the amino acid sequences of SEQ ID NOs: 112 and 115, respectively; SEQ ID NOs: 121 and 124, respectively; SEQ ID NOs: 103 and 106, respectively; or SEQ ID NOs: 130 and 106, respectively, or any antibody or antigen-binding fragment thereof that has at least at or about 90% sequence identity to any of the above V_{H} and V_{L}, such as at least at or about 90%, 91%, 92%, 93%, 94%, 95%, 96%, 97%, 98%, or 99% sequence identity thereto. For example, the V_{H} and V_{L} regions of the antibody or antigen-binding fragment thereof provided therein comprise the amino acid sequences selected from: SEQ ID NOs: 112 and 115; SEQ ID NOs: 121 and 124; SEQ ID NOs: 103 and 106; or SEQ ID NOs: 130 and 106, respectively.

In some embodiments of the antibody, antigen-binding fragment, receptor, e.g., CAR, conjugates or binding molecules provided herein, the V_{H} region and the V_{L} region are or comprise the sequence set forth in SEQ ID NOS: 112 and 115, respectively. In some embodiments of the antibody, antigen-binding fragment, receptor, e.g., CAR, conjugates or binding molecules provided herein, the V_{H} region and the V_{L} region are or comprise the sequence set forth in SEQ ID NOS: 121 and 124, respectively. In some embodiments of the antibody, antigen-binding fragment, receptor, e.g., CAR, conjugates or binding molecules provided herein, the V_{H} region and the V_{L} region are or comprise the sequence set forth in SEQ ID NOS: 103 and 106, respectively. In some embodiments of the antibody, antigen-binding fragment, receptor, e.g., CAR, conjugates or binding molecules provided herein, the V_{H} region and the V_{L} region are or comprise the sequence set forth in SEQ ID NOS: 130 and 106, respectively.

In some embodiments of the antibody, antigen-binding fragment, receptor, e.g., CAR, conjugates or binding molecules provided herein, the V_{H} region is or comprises the amino acid sequence encoded by SEQ ID NO: 110, 119, 101 or 128 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 110, 119, 101 or 128, and the V_{L} region is or comprises the amino acid sequence encoded by SEQ ID NO: 113, 122 or 104, or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 113, 122 or 104. In some embodiments of the antibody, antigen-binding fragment, receptor, e.g., CAR, conjugates or binding molecules provided herein, the V_{H} region is or comprises the amino acid sequence encoded by SEQ ID NO: 111, 120, 102 or 129, and the V_{L} region is or comprises the amino acid sequence encoded by SEQ ID NO: 114, 123, 105 or 131.

In some embodiments of the antibody, antigen-binding fragment, receptor, e.g., CAR, conjugates or binding molecules provided herein, the V_{H} region is or comprises the amino acid sequence encoded by SEQ ID NO: 110 or 119 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 110 or 119, and the V_{L} region is or comprises the amino acid sequence encoded by SEQ ID NO: 113 or 122, or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 113 or 122. In some embodiments of the antibody, antigen-binding fragment, receptor, e.g., CAR, conjugates or binding molecules provided herein, the V_{H} region is or comprises the amino acid sequence encoded by SEQ ID NO: 111 or 120, and the V_{L} region is or comprises the amino acid sequence encoded by SEQ ID NO: 114 or 123.

In some embodiments of the antibody, antigen-binding fragment, receptor, e.g., CAR, conjugates or binding molecules provided herein, the V_{H} region is or comprises the amino acid sequence encoded by SEQ ID NO: 110 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 110, and the V_{L} region is or comprises the amino acid sequence encoded by SEQ ID NO: 113, or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 113. In some embodiments of the antibody, antigen-binding fragment, receptor, e.g., CAR, conjugates or binding molecules provided herein, the V_{H} region is or comprises the amino acid sequence encoded by SEQ ID NO: 111, and the V_{L} region is or comprises the amino acid sequence encoded by SEQ ID NO: 114.

In some embodiments of the antibody, antigen-binding fragment, receptor, e.g., CAR, conjugates or binding molecules provided herein, the V_{H} region is or comprises the amino acid sequence encoded by SEQ ID NO: 119 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 119, and the V_{L} region is or comprises the amino acid sequence encoded by SEQ ID NO: 122, or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 122. In some embodiments of the antibody, antigen-binding fragment, receptor, e.g., CAR, conjugates or binding molecules provided herein, the V_{H} region is or comprises the amino acid sequence encoded by SEQ ID NO: 120, and the V_{L} region is or comprises the amino acid sequence encoded by SEQ ID NO: 123.

In some embodiments of the antibody, antigen-binding fragment, receptor, e.g., CAR, conjugates or binding molecules provided herein, the V_{H} region is or comprises the amino acid sequence encoded by SEQ ID NO: 101 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 101, and the V_{L} region is or comprises the amino acid sequence encoded by SEQ ID NO: 104, or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 104. In some embodiments of the antibody, antigen-binding fragment, receptor, e.g., CAR, conjugates or binding molecules provided herein, the V_{H} region is or comprises the amino acid sequence encoded by SEQ ID NO: 102 and the V_{L} region is or comprises the amino acid sequence encoded by SEQ ID NO: 105.

In some embodiments of the antibody, antigen-binding fragment, receptor, e.g., CAR, conjugates or binding molecules provided herein, the V_{H} region is or comprises the amino acid sequence encoded by SEQ ID NO: 128 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 128 and the V_{L} region is or comprises the amino acid sequence encoded by SEQ ID NO: 104, or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 104. In some embodiments of the antibody, antigen-binding fragment, receptor, e.g., CAR, conjugates or binding molecules provided herein, the V_{H} region is or comprises the amino acid sequence encoded by SEQ ID NO: 129, and the V_{L} region is or comprises the amino acid sequence encoded by SEQ ID NO: 131.

In some embodiments, the antibody or antigen-binding fragment thereof, in the provided CAR, is a single-chain antibody fragment, such as a single chain variable fragment (scFv) or a diabody or a single domain antibody (sdAb). In some embodiments, the antibody or antigen binding fragment is a multi-domain antibody, such as an scFv comprising a heavy chain variable (V_{H}) region and a light chain variable (V_{L}) region. In some embodiments, the single-chain antibody fragment (e.g., scFv) includes one or more linkers joining two antibody domains or regions, such as a heavy chain variable (V_{H}) region and a light chain variable (V_{L}) region. The linker typically is a peptide linker, e.g., a flexible and/or soluble peptide linker. Among the linkers are those rich in glycine and serine and/or in some cases threonine. In some embodiments, the linkers further include charged residues such as lysine and/or glutamate, which can improve solubility. In some embodiments, the linkers further include one or more proline.

Accordingly, the provided CARs contain anti-ROR1 antibodies that include single-chain antibody fragments, such as scFvs and diabodies, particularly human single-chain antibody fragments, typically comprising linker(s) joining two antibody domains or regions, such V_{H} and V_{L} regions. The linker typically is a peptide linker, e.g., a flexible and/or soluble peptide linker, such as one rich in glycine and serine.

In some aspects, the linkers rich in glycine and serine (and/or threonine) include at least 80%, 85%, 90%, 91%, 92%, 93%, 94%, 95%, 96%, 97%, 98%, or 99% such amino acid(s). In some embodiments, they include at least at or about 50%, 55%, 60%, 70%, or 75%, glycine, serine, and/or threonine. In some embodiments, the linker is comprised substantially entirely of glycine, serine, and/or threonine. The linkers generally are between about 5 and about 50 amino acids in length, typically between at or about 10 and at or about 30, e.g., 10, 11, 12, 13, 14, 15, 16, 17, 18, 19, 20, 21, 22, 23, 24, 25, 26, 27, 28, 29, or 30, and in some examples between 10 and 25 amino acids in length. Exemplary linkers include linkers having various numbers of repeats of the sequence GGGGS (4GS; SEQ ID NO: 39) or GGGS (3GS; SEQ ID NO: 40), such as between 2, 3, 4 and 5 repeats of such a sequence. Exemplary linkers include those having or consisting of an sequence set forth in SEQ ID NO: 41 (GGGGSGGGGSGGGGS). Exemplary linkers further include those having or consisting of the sequence set forth in SEQ ID NO: 24 (GSTSGSGKPGSGEGSTKG). Exemplary linkers further include those having or consisting of the sequence set forth in SEQ ID NO: 162 (SRGGGGSGGGGSGGGGSLEMA). An exemplary linker includes those having or consisting of the sequence set forth in SEQ ID NO; 163 (GSRGGGGSGGGGSGGGGSLEMA).

Accordingly, in some embodiments, the provided embodiments include single-chain antibody fragments, e.g., scFvs, comprising one or more of the aforementioned linkers, such as glycine/serine rich linkers, including linkers having repeats of GGGS (SEQ ID NO: 40) or GGGGS (SEQ ID NO: 39), such as the linker set forth in SEQ ID NO: 41, 162 or 163.

In some embodiments, the V_{H} region may be amino terminal to the V_{L} region. In some embodiments, the V_{H} region may be carboxy terminal to the V_{L} region. In particular embodiments, the fragment, e.g., scFv, may include a V_{H} region or portion thereof, followed by the linker, followed by a V_{L} region or portion thereof. In other embodiments, the fragment, e.g., the scFv, may include the V_{L} region or portion thereof, followed by the linker, followed by the V_{H} region or portion thereof.

In some embodiments of the antibody, antigen-binding fragment, receptor, e.g., CAR, conjugates or binding molecules provided herein, the scFv is or comprises the sequence set forth in SEQ ID NO: 118, 127, 109 or 134, or an amino acid sequence having at least at or about 85%, 86%, 87%, 88%, 89%, 90%, 91%, 92%, 93%, 94%, 95%, 96%, 97%, 98% or 99% sequence identity to SEQ ID NO: 118, 127, 109 or 134. In some embodiments of the antibody, antigen-binding fragment, receptor, e.g., CAR, conjugates or binding molecules provided herein, the scFv is or comprises the sequence set forth in SEQ ID NO: 118 or 127 or an amino acid sequence having at least at or about 85%, 86%, 87%, 88%, 89%, 90%, 91%, 92%, 93%, 94%, 95%, 96%, 97%, 98% or 99% sequence identity to SEQ ID NO: 118 or 127. In some of any of the provided embodiments, the scFv is or comprises the sequence set forth in SEQ ID NO: 118. In some of any of the provided embodiments, the scFv is or comprises the sequence set forth in SEQ ID NO: 127. In some of any of the provided embodiments, the scFv is or comprises the sequence set forth in SEQ ID NO: 109. In some of any of the provided embodiments, the scFv is or comprises the sequence set forth in SEQ ID NO: 134.

In some embodiments of the antibody, antigen-binding fragment, receptor, e.g., CAR, conjugates or binding molecules provided herein, the scFv is or comprises the amino acid sequence encoded by SEQ ID NO: 116, 125, 107 or 132 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 116, 125, 107 or 132. In some of any of the provided embodiments, the scFv is or comprises the amino acid sequence encoded by SEQ ID NO: 117, 126, 108 or 133.

In some of any of the provided embodiments, the scFv is or comprises the amino acid sequence encoded by SEQ ID NO: 116 or 125 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 116 or 125. In some embodiments, the scFv is or comprises the amino acid sequence encoded by SEQ ID NO: 117 or 126. In some embodiments, the scFv is or comprises the amino acid sequence encoded by SEQ ID NO: 117. In some embodiments, the scFv is or comprises the amino acid sequence encoded by SEQ ID NO: 126. In some embodiments, the scFv is or comprises the amino acid sequence encoded by SEQ ID NO: 108. In some embodiments, the scFv is or comprises the amino acid sequence encoded by SEQ ID NO: 133

Table 2 provides the SEQ ID NOS: of exemplary provided antibody fragments, such as scFvs. In some aspects, the exemplary provided antibody fragments can be comprised in the provided ROR1-binding receptors, such as anti-ROR1 chimeric antigen receptors (CARs). In some embodiments, the ROR1-binding antibody or fragment thereof, such as an scFv, comprises a V_{H} region that comprises the CDR-H1, the CDR-H2 and the CDR-H3 sequence and a V_{L} region that comprises the CDR-L1, the CDR-L2 and the CDR-L3 sequence set forth in the SEQ ID NOS: listed in each row of Table 2 below (by Kabat, Chothia, AbM and IgBLAST numbering schemes). In some embodiments, the ROR1-binding antibody or fragment thereof, such as an scFv, comprises a V_{H} region sequence and a V_{L} region sequence set forth in the SEQ ID NOS: listed in each row of Table 2 below, or an antibody comprising a V_{H} region and a V_{L} region amino acid sequence that has at least 90%, 91%, 92%, 93%, 94%, 95%, 96%, 97%, 98%, or 99% sequence identity to the V_{H} region sequence and the V_{L} region sequence set forth in the SEQ ID NOS: listed in each row of Table 2 below. In some embodiments, the ROR1-binding antibody or fragment thereof, such as an scFv, comprises a V_{H} region sequence and a V_{L} region sequence set forth in the SEQ ID NOS: listed in each row of Table 2 below. In some embodiments, the ROR1-binding antibody or fragment thereof comprises an scFv sequence set forth in the SEQ ID NOS: listed in each row of Table 2 below, or an antibody comprising an scFv amino acid sequence that has at least at or about 85%, 86%, 87%, 88%, 89%, 90%, 91%, 92%, 93%, 94%, 95%, 96%, 97%, 98%, or 99% sequence identity to the scFv sequence set forth in the SEQ ID NOS: listed in each row of Table 2 below. In some embodiments, the ROR1-binding antibody or fragment thereof comprises an scFv sequence set forth in the SEQ ID NOS: listed in each row of Table 2 below. In some embodiments, any of the antibody or antigen-binding fragment thereof, such as scFv, listed in each row of Table 2 can be comprised in a receptor, such as a chimeric antigen receptor (CAR), for example, as the extracellular antigen-binding domain.

In some embodiments, the provided antibody or antigen-binding fragment thereof comprises a V_{H} region and a V_{L} region, wherein the V_{H} region of the antibody or antigen-binding fragment thereof can contain a combination of any of the CDR-H1, the CDR-H2 and the CDR-H3 amino acid sequences set forth in Table 2, and the V_{L} region of the antibody or antigen-binding fragment thereof can contain a combination of any of the CDR-L1, the CDR-L2 and the CDR-L3 amino acid sequences set forth in Table 2. In some embodiments, the provided antibody or antigen-binding fragment thereof comprises a V_{H} region and/or a V_{L} region set forth in Table 2, in any combination, orientation or containing a different linker. In some aspects, the antibody or antigen-binding fragment thereof comprises a V_{H} region described in Table 2. In some aspects, the antibody or antigen-binding fragment thereof comprises a V_{L} region described in Table 2. In some embodiments, the provided antibody or antigen-binding fragment thereof is a single-domain antibody (sdAb), comprising a V_{H} region set forth in Table 2, or a V_{L} region set forth in Table 2. In some embodiments, the provided antibody or antigen-binding fragment thereof is a single-domain antibody (sdAb), comprising a V_{H} region containing a CDR-H1, a CDR-H2 and/or a CDR-H3 set forth in Table 2, or a V_{L} region containing a CDR-L1, a CDR-L2 and/or a CDR-L3 set forth in Table 2.

TABLE 2
Sequence identifier (SEQ ID NO) for Exemplary scFvs

| scFv | CDR-H1 | CDR-H2 | CDR-H3 | CDR-L1 | CDR-L2 | CDR-L3 | V_{H} | V_{L} | Linker | Order | scFv |
|---|---|---|---|---|---|---|---|---|---|---|---|
| | Kabat | | | Kabat | | | | | | | |
| ROR1-1 | 67 | 71 | 73 | 75 | 77 | 79 | 112 | 115 | 41 | V_{H}-V_{L} | 118 |
| ROR1-2 | 82 | 86 | 88 | 90 | 92 | 94 | 121 | 124 | 41 | V_{H}-V_{L} | 127 |
| ROR1-3 | 52 | 56 | 58 | 60 | 62 | 64 | 103 | 106 | 41 | V_{H}-V_{L} | 109 |
| ROR1-4 | 52 | 97 | 99 | 60 | 62 | 64 | 130 | 106 | 41 | V_{H}-V_{L} | 134 |
| | Chothia | | | Chothia | | | | | | | |
| ROR1-1 | 65 | 69 | 73 | 75 | 77 | 79 | 112 | 115 | 41 | V_{H}-V_{L} | 118 |
| ROR1-2 | 80 | 84 | 88 | 90 | 92 | 94 | 121 | 124 | 41 | V_{H}-V_{L} | 127 |
| ROR1-3 | 50 | 54 | 58 | 60 | 62 | 64 | 103 | 106 | 41 | V_{H}-V_{L} | 109 |
| ROR1-4 | 50 | 95 | 99 | 60 | 62 | 64 | 130 | 106 | 41 | V_{H}-V_{L} | 134 |
| | AbM | | | AbM | | | | | | | |
| ROR1-1 | 66 | 70 | 73 | 75 | 77 | 79 | 112 | 115 | 41 | V_{H}-V_{L} | 118 |
| ROR1-2 | 81 | 85 | 88 | 90 | 92 | 94 | 121 | 124 | 41 | V_{H}-V_{L} | 127 |
| ROR1-3 | 51 | 55 | 58 | 60 | 62 | 64 | 103 | 106 | 41 | V_{H}-V_{L} | 109 |
| ROR1-4 | 51 | 96 | 99 | 60 | 62 | 64 | 130 | 106 | 41 | V_{H}-V_{L} | 134 |
| | IgBLAST | | | IgBLAST | | | | | | | |
| ROR1-1 | 68 | 72 | 74 | 76 | 78 | 79 | 112 | 115 | 41 | V_{H}-V_{L} | 118 |
| ROR1-2 | 83 | 87 | 89 | 91 | 93 | 94 | 121 | 124 | 41 | V_{H}-V_{L} | 127 |
| ROR1-3 | 53 | 57 | 59 | 61 | 63 | 64 | 103 | 106 | 41 | V_{H}-V_{L} | 109 |
| ROR1-4 | 53 | 98 | 100 | 61 | 63 | 64 | 130 | 106 | 41 | V_{H}-V_{L} | 134 |

Among the provided antibodies, e.g., antigen-binding fragments, are human antibodies. In some embodiments of a provided human anti-ROR1 antibody, e.g., antigen-binding fragments, the human antibody contains a V_{H} region that comprises a portion having at least 95%, 96%, 97%, 98%, 99%, or 100% sequence identity to an amino acid sequence encoded by a germline nucleotide human heavy chain V segment, a portion having at least 95%, 96%, 97%, 98%, 99%, or 100% sequence identity to an amino acid sequence encoded by a germline nucleotide human heavy chain D segment, and/or a portion having at least 95%, 96%, 97%, 98%, 99%, or 100% sequence identity to an amino acid sequence encoded by a germline nucleotide human heavy chain J segment; and/or contains a V_{L} region that comprises a portion having at least 95%, 96%, 97%, 98%, 99%, or 100% sequence identity to an amino acid sequence encoded by a germline nucleotide human kappa or lambda chain V segment, and/or a portion having at least 95%, 96%, 97%, 98%, 99%, or 100% sequence identity to an amino acid sequence encoded by a germline nucleotide human kappa or lambda chain J segment.

Among the provided antibodies, e.g., antigen-binding fragments, are human antibodies. In some embodiments of a provided human anti-ROR1 antibody, e.g., antigen-binding fragments, the human antibody contains a V_{H} region that comprises a portion having at least 95%, 96%, 97%, 98%, 99%, or 100% sequence identity to an amino acid sequence encoded by a germline nucleotide human heavy chain V segment, a portion having at least 95%, 96%, 97%, 98%, 99%, or 100% sequence identity to an amino acid sequence encoded by a germline nucleotide human heavy chain D segment, and/or a portion having at least 95%, 96%, 97%, 98%, 99%, or 100% sequence identity to an amino acid sequence encoded by a germline nucleotide human heavy chain J segment; and contains a V_{L} region that comprises a portion having at least 95%, 96%, 97%, 98%, 99%, or 100% sequence identity to an amino acid sequence encoded by a germline nucleotide human kappa or lambda chain V segment, and/or a portion having at least 95%, 96%, 97%, 98%, 99%, or 100% sequence identity to an amino acid sequence encoded by a germline nucleotide human kappa or lambda chain J segment. In some embodiments, the portion of the V_{H} region corresponds to the CDR-H1, the CDR-H2 and/or the CDR-H3. In some embodiments, the portion of the V_{H} region corresponds to the CDR-H1, the CDR-H2 and the CDR-H3. In some embodiments, the portion of the V_{H} region corresponds to the framework region 1 (FR1), FR2, FR2 and/or FR4. In some embodiments, the portion of the V_{L} region corresponds to the CDR-L1, the CDR-L2 and/or the CDR-L3. In some embodiments, the portion of the V_{L} region corresponds to the CDR-L1, the CDR-L2 and the CDR-L3. In some embodiments, the portion of the V_{L} region corresponds to the FR1, FR2, FR2 and/or FR4.

In some embodiments, the human antibody or antigen-binding fragment thereof, contains a CDR-H1 having at least 95%, 96%, 97%, 98%, 99%, or 100% sequence identity to an amino acid sequence of the corresponding CDR-H1 region within a sequence encoded by a germline nucleotide human heavy chain V segment. For example, the human antibody in some embodiments contains a CDR-H1 having a sequence that is 100% identical or with no more than one, two or three amino acid differences as compared to the corresponding CDR-H1 region within a sequence encoded by a germline nucleotide human heavy chain V segment.

In some embodiments, the human antibody or antigen-binding fragment thereof, contains a CDR-H2 having at least 95%, 96%, 97%, 98%, 99%, or 100% sequence identity to an amino acid sequence of the corresponding CDR-H2 region within a sequence encoded by a germline nucleotide human heavy chain V segment. For example, the human antibody in some embodiments contains a CDR-H2 having a sequence that is 100% identical or with no more than one, two or three amino acid difference as compared to the corresponding CDR-H2 region within a sequence encoded by a germline nucleotide human heavy chain V segment.

In some embodiments, the human antibody or antigen-binding fragment thereof, contains a CDR-H3 having at least 95%, 96%, 97%, 98%, 99%, or 100% sequence identity to an amino acid sequence of the corresponding CDR-H3 region within a sequence encoded by a germline nucleotide human heavy chain V segment, D segment and J segment. For example, the human antibody in some embodiments contains a CDR-H3 having a sequence that is 100% identical or with no more than one, two or three amino acid differences as compared to the corresponding CDR-H3 region within a sequence encoded by a germline nucleotide human heavy chain V segment, D segment and J segment.

In some embodiments, the human antibody or antigen-binding fragment thereof, contains a CDR-L1 having at least 95%, 96%, 97%, 98%, 99%, or 100% sequence identity to an amino acid sequence of the corresponding CDR-L1 region within a sequence encoded by a germline nucleotide human light chain V segment. For example, the human antibody in some embodiments contains a CDR-L1 having a sequence that is 100% identical or with no more than one, two or three amino acid differences as compared to the corresponding CDR-L1 region within a sequence encoded by a germline nucleotide human light chain V segment.

In some embodiments, the human antibody or antigen-binding fragment thereof, contains a CDR-L2 having at least 95%, 96%, 97%, 98%, 99%, or 100% sequence identity to an amino acid sequence of the corresponding CDR-L2 region within a sequence encoded by a germline nucleotide human light chain V segment. For example, the human antibody in some embodiments contains a CDR-L2 having a sequence that is 100% identical or with no more than one, two or three amino acid difference as compared to the corresponding CDR-L2 region within a sequence encoded by a germline nucleotide human light chain V segment.

In some embodiments, the human antibody or antigen-binding fragment thereof, contains a CDR-L3 having at least 95%, 96%, 97%, 98%, 99%, or 100% sequence identity to an amino acid sequence of the corresponding CDR-L3 region within a sequence encoded by a germline nucleotide human light chain V segment and J segment. For example, the human antibody in some embodiments contains a CDR-L3 having a sequence that is 100% identical or with no more than one, two or three amino acid differences as compared to the corresponding CDR-L3 region within a sequence encoded by a germline nucleotide human light chain V segment and J segment.

In some embodiments, the human antibody or antigen-binding fragment thereof, contains a framework region that contains human germline gene segment sequences. For example, in some embodiments, the human antibody contains a V_{H} region in which the framework region, e.g. FR1, FR2, FR3 and FR4, has at least 95%, 96%, 97%, 98%, 99%, or 100% sequence identity to a framework region encoded by a human germline antibody segment, such as a V segment and/or J segment. In some embodiments, the human antibody contains a V_{L} region in which the framework region e.g. FR1, FR2, FR3 and FR4, has at least 95%, 96%, 97%, 98%, 99%, or 100% sequence identity to a framework region encoded by a human germline antibody segment, such as a V segment and/or J segment. For example, in some such embodiments, the framework region sequence contained within the V_{H} region and/or V_{L} region differs by no more than 10 amino acids, such as no more than 9, 8, 7, 6, 5, 4, 3, 2 or 1 amino acid, compared to the framework region sequence encoded by a human germline antibody segment.

The antibody or antigen-binding fragment thereof, may contain at least a portion of an immunoglobulin constant region, such as one or more constant region domain. In some embodiments, the constant regions include a light chain constant region and/or a heavy chain constant region 1 (C_{H}1). In some embodiments, the antibody includes at least a portion of a hinge region or a variant thereof. In some embodiments, the antibody includes a C_{H}2 and/or C_{H}3 domain, such as an Fc region. In some embodiments, the Fc region is an Fc region of a human IgG, such as an IgG1 or IgG4.

### 2. Exemplary Features

In some aspects, the provided antibodies have one or more specified functional features, such as binding properties, including binding to particular epitopes or exhibiting lower or reduced binding to a related but non-specific antigen. In some aspects, the provided antibodies can bind to an epitope that is similar to or overlaps with epitopes of other antibodies, such as reference antibodies, and/or exhibit particular binding affinities. In some aspects, the provided antibodies can bind to an epitope that is different from epitopes of other antibodies, e.g., binding a conformational epitope.

In some embodiments, the provided antibodies or antigen-binding fragment thereof specifically bind to a receptor tyrosine kinase-like orphan receptor 1 (ROR1) protein. In some of any of the embodiments provided herein, ROR1 refers to human ROR1. The observation that an antibody or other binding molecule binds to ROR1 or specifically binds to ROR1 does not necessarily mean that it binds to ROR1 from every species. For example, in some embodiments, features of binding to ROR1, such as the ability to specifically bind thereto and/or to compete for binding thereto with a reference antibody, and/or to bind with a particular affinity or compete to a particular degree, in some embodiments, refers to the ability with respect to a human ROR1 protein and the antibody may not have this feature with respect to a ROR1 of another species such as mouse. In some embodiments, the antibody binds to human ROR1 and binds to ROR1 of another species, such as Rhesus macaque or cynomolgus macaque. In some embodiments, the antibody or an antigen-binding fragment thereof binds to human ROR1 and does not bind to ROR1 of another species, such as mouse. In some embodiments, the antibody binds to human ROR1 and binds to ROR1 of another species, such as mouse.

In some embodiments, the antibodies, such as the anti-ROR1 antibodies, e.g., the human antibodies, specifically bind to a particular epitope or region of ROR1, such as generally an extracellular epitope or region. ROR1 is a type I membrane protein that contains an extracellular region containing an immunoglobulin (Ig) domain, a frizzled (Fz) domain and a kringle (Kr) domain followed by a transmembrane domain. With reference to human ROR1 set forth in SEQ ID NO:144 (GenBank No. AAA60275.1; sequence including the signal peptide set forth in SEQ ID NO:215, Uniprot No. Q01973), the extracellular region corresponds to amino acids 1-377, amino acids 13-118 correspond to the Ig domain, amino acids 136-270 correspond to the Fz domain and amino acids 283-362 correspond to the Kr domain (corresponding to amino acids 42-147 for the Ig domain, amino acids 165-299 for the Fz domain and amino acids 312-391 for the Kr domain with reference to amino acid sequence set forth in SEQ ID NO:215). In some embodiments, the antibodies, such as human antibodies, bind to an epitope comprising residues within the Ig domain, the Fz domain and/or the Kr domain. In some embodiments, the antibodies, such as human antibodies, bind to an epitope comprising residues with the Ig domain and/or Fz domain. In some embodiments, the antibodies, such as human antibodies, bind to an epitope comprising residues within both the Ig and Fz domains.

In some embodiments, the antibodies or antigen-binding fragment thereof bind, such as specifically bind, to human ROR1, such as to one or more epitopes or region of human ROR1, such as the human ROR1 set forth in SEQ ID NO:144 (GenBank No. AAA60275.1; sequence including the signal peptide set forth in SEQ ID NO:215, Uniprot No. Q01973), or an allelic variant or splice variant thereof. In some embodiments, the antibodies or antigen-binding fragment thereof specifically binds to one or more epitopes within a human ROR1 protein. In one embodiment, human ROR1 is a transcript variant or isoform that has the sequence of amino acids forth in SEQ ID NO:145 or 146. In some embodiments, human ROR1 protein comprises an amino acid sequence set forth in SEQ ID NO: 144, 145, 146 or 215. In some embodiments, the antibodies or antigen-binding fragment thereof bind to the extracellular region ROR1, such as to one or more extracellular epitopes present within the extracellular region of human ROR1, e.g., corresponding to residues 1-377 of the human ROR1 sequence set forth in SEQ ID NO:144 (corresponding to residues 30-406 of the human ROR1 sequence set forth in SEQ ID NO:215 that includes the signal peptide).

In some embodiments, the antibodies or antigen-binding fragment thereof bind one or more epitope of ROR1, such as a human ROR1. In some embodiments, the antibodies or antigen-binding fragment thereof bind a linear epitope of ROR1, such as a human ROR1. In some embodiments, the antibodies or antigen-binding fragment thereof bind one or more conformational epitopes of ROR1, such as a human ROR1.

In some embodiments, the antibodies or antigen-binding fragment thereof bind one or more epitopes of human ROR1, such as one or more epitopes comprising or consisting of an amino acid sequence selected from among any one of SEQ ID NOS: 199-214, or one or more epitopes present within an amino acid sequence selected from among any one of SEQ ID NOS: 199-214. In some embodiments, the antibodies or antigen-binding fragment thereof bind one or more epitopes of human ROR1, such as one or more epitopes that include the sequence FRSTIYGSRLRIRNL (set forth in SEQ ID NO:199). In some embedment's, the anti-ROR1 antibody or antigen-binding fragment thereof specifically binds to an epitope consisting of the sequence set forth in SEQ ID NO:199 or an epitope present within the sequence set forth in SEQ ID NO:199. In some embodiments, the antibodies or antigen-binding fragment thereof bind additional epitopes, such as one or more conformational epitopes. In some embodiments, the antibodies or antigen-binding fragment thereof bind additional epitopes, in addition to the sequence FRSTIYGSRLRIRNL (set forth in SEQ ID NO:199). Exemplary of one or more additional epitopes include, but are not limited to, one or more of the sequences set forth in SEQ ID NO:200-214 or one or more epitopes present within an amino acid sequence selected from among any one of SEQ ID NOS: 200-214.

In some embodiments, the antibody binds to non-human ROR1, such as Rhesus macaques (Macaca mulatta) ROR1 (set forth in SEQ ID NO:216, Uniprot No. F6RUP2) or cynomolgus macaques (Macaca fasicularis) ROR1 (set forth in SEQ ID NO:217, Uniprot No. AOA2K5WTX7; or SEQ ID NO:218, Uniprot No. AOA2K5WTX4). In some aspects, the extracellular domain of the non-human ROR1 is at least 99% identical to the human ROR1 sequence.

In some embodiments, the antibody binds to non-human ROR1, such as monkey, rabbit, rat, mouse, or other species of ROR1. In some embodiments, the antibody binds to mouse (Mus musculus) ROR1, such as to an epitope or region of mouse ROR1, such as the mouse ROR1 set forth in SEQ ID NO: 171 (GenBank No. NP_038873; sequence including the signal peptide set forth in SEQ ID NO:219, Uniprot No. Q9Z139). In some embodiments, the antibody binds to human ROR1 and binds to mouse ROR1. In some embodiments, the extent of binding of some of the provided anti-ROR1 antibodies or fragments thereof to a non-human ROR1, such as mouse ROR1, is at least at or about 75%, 80%, 90%, 95%, 100%, 110%, 120%, 130%, 140%, 150% or more of the binding of the antibody to human ROR1.

In some of any of the provided embodiments, the antibody or antigen-binding fragment thereof does not bind to, is not cross-reactive to, or binds at a lower extent, level or degree or affinity to a non-human ROR1, optionally a mouse ROR1. In some embodiments, the extent of binding of an anti-ROR1 antibody to an unrelated, non-ROR1 protein or to a non-human ROR1 protein, such as a mouse ROR1 protein, or other non-ROR1 protein, is less than at or about 50%, 40%, 30%, 20% or 10% of the binding of the antibody to human ROR1 as measured. In some embodiments, the antibodies or antigen-binding fragments thereof do not bind to mouse ROR1, such as the mouse ROR1 set forth in SEQ ID NO:171 or 219. In some embodiments, the antibodies or antigen-binding fragments do not bind to, is not cross-reactive to, or binds at a lower level or degree or affinity to a mouse ROR1. In some embodiments, the extent, level or degree or affinity of binding of the provided anti-ROR1 antibody or antigen-binding fragment thereof to a mouse ROR1 is at least at or about 75%, 80%, 90%, 95% or 99% less than the extent, level or degree or affinity of binding to a human ROR1.

In some of any of the provided embodiments, the antibody or antigen-binding fragment thereof does not bind to, is not cross-reactive to, or binds at a lower extent, level or degree or affinity to a receptor tyrosine kinase-like orphan receptor 2 (ROR2) protein, optionally a human ROR2 protein. In some embodiments, the extent of binding of some of the provided anti-ROR1 antibodies or fragments thereof to a non-ROR1 protein, such as a ROR2 protein, is at least at or about 75%, 80%, 90%, 95% or 99% less than the binding of the antibody to human ROR1. In some embodiments, the provided antibody or antigen-binding fragment thereof does not bind to, is not cross-reactive to, or binds at a lower level or degree or affinity to a ROR2 protein, optionally a human ROR2 protein. In some embodiments, among provided antibodies are antibodies in which binding to mouse ROR1 is less than or at or about 30%, 20% or 10%, such as less than at or about 10%, of the binding of the antibody to human ROR1. In some embodiments, among provided antibodies are antibodies in which binding to a ROR2, such as a human ROR2, is less than or at or about 30%, 20% or 10%, such as less than at or about 10%, of the binding of the antibody to human ROR1.

In some embodiments, the provided antibodies are capable of binding ROR1, such as human ROR1, with at least a certain affinity, as measured by any of a number of known methods. In some embodiments, the affinity is represented by an equilibrium dissociation constant (K_{D}). In some embodiments, the affinity is represented by EC₅₀.

A variety of assays are known for assessing binding affinity, equilibrium dissociation constant (K_{D}), equilibrium association constant (K_{A}), EC₅₀, on-rate (association rate constant; kₒₙ or k_{d}; units of 1/Ms or M⁻¹s⁻¹) and the off-rate (dissociation rate constant; k_{off} or k_{d}; units of 1/s or s⁻¹) and/or determining whether a binding molecule (e.g., an antibody or fragment thereof) specifically binds to a particular ligand (e.g., an antigen, such as a ROR1 protein). One can determine the binding affinity of a binding molecule, e.g., an antibody or an antigen-binding fragment thereof, for an antigen, e.g., ROR1, such as human ROR1 or cynomolgus ROR1 or mouse ROR1, such as by using any of a number of binding assays that are well known. For example, in some embodiments, a BIAcore® instrument can be used to determine the binding kinetics and constants of a complex between two proteins (e.g., an antibody or fragment thereof, and an antigen, such as a ROR1 protein), using surface plasmon resonance (SPR) analysis (see, e.g., Scatchard et al., Ann. N. Y. Acad. Sci. 51:660, 1949; Wilson, Science 295:2103, 2002; Wolff et al., Cancer Res. 53:2560, 1993; and U.S. Pat. Nos. 5,283,173, 5,468,614, or the equivalent).

SPR measures changes in the concentration of molecules at a sensor surface as molecules bind to or dissociate from the surface. The change in the SPR signal is directly proportional to the change in mass concentration close to the surface, thereby allowing measurement of binding kinetics between two molecules. The dissociation rate constant (k_{off} or kₐ), the association rate constant (kₒₙ or kₐ) and/or equilibrium dissociation constant (K_{D}) and/or equilibrium association constant (K_{A}) for the complex can be determined by monitoring changes in the refractive index with respect to time as buffer is passed over the chip. Other suitable assays for measuring the binding of one protein to another include, for example, immunoassays such as enzyme linked immunosorbent assays (ELISA) and radioimmunoassays (RIA), or determination of binding by monitoring the change in the spectroscopic or optical properties of the proteins through fluorescence, UV absorption, circular dichroism, or nuclear magnetic resonance (NMR). Other exemplary assays include, but are not limited to, Western blot, ELISA, analytical ultracentrifugation, spectroscopy, flow cytometry, sequencing, genetic reporter assays, flow cytometry, and other methods for detection of expressed nucleic acids or binding of proteins.

In some embodiments, the binding molecule, e.g., antibody or fragment thereof, binds, such as specifically binds, to an antigen, e.g., a ROR1 protein or an epitope therein, with an affinity or K_{A} (i.e., an equilibrium association constant of a particular binding interaction with units of 1/M or M⁻¹; equal to the ratio of the on-rate [kₒₙ or kₐ] to the off-rate [k_{off} or kₐ] for this association reaction, assuming bimolecular interaction) equal to or greater than 10⁵ M⁻¹. In some embodiments, the peptide binding molecule binds, such as specifically binds, to an epitope of an antigen, e.g., human ROR1, with an affinity or K_{A} (i.e., an equilibrium association constant of a particular binding interaction with units of 1/M or M⁻¹) equal to or greater than 10⁵ M^{A} (which equals the ratio of the on-rate [kₒₙ] to the off-rate [k_{off}] for this association reaction). In some embodiments, the binding molecule, e.g., antibody or antigen-binding fragment thereof, exhibits a binding affinity for a T cell epitope of the target polypeptide with an affinity or K_{A} ranging from at or about 10⁶ M⁻¹ to at or about 10¹⁰ M⁻¹, such as from at or about 10⁶ M⁻¹ to at or about 10⁹ M⁻¹, or from at or about 10⁶ M⁻¹ to at or about 10⁻⁸ M. In some embodiments, binding affinity may be classified as high affinity or as low affinity. For example, in some cases, a binding molecule, e.g., antibody or antigen-binding fragment thereof, that exhibits high affinity binding to a particular epitope interacts with such epitope with a K_{A} of at least at or about 107 M⁻¹, at least at or about 10⁸ M⁻¹, at least at or about 10⁹ M⁻¹, at least at or about 10¹⁰ M⁻¹, at least at or about 10¹¹ M⁻¹, at least at or about 10¹² M⁻¹, or at least at or about 10¹³ M⁻¹. In some cases, a binding molecule, e.g., antibody or antigen-binding fragment thereof, that exhibits low affinity binding exhibits a K_{A} of up to 107 M⁻¹, up to 10⁶ M⁻¹, up to 10⁵ M⁻¹.

Alternatively, affinity can be defined as an equilibrium dissociation constant (K_{D}) of a particular binding interaction with units of M (e.g., 10⁻⁵ M to 10⁻¹ M). In some embodiments, the antibody or fragment thereof exhibits a binding affinity for the epitope with a K_{D} (i.e., an equilibrium dissociation constant of a particular binding interaction with units of M; equal to the ratio of the off-rate [k_{off} or kₐ] to the on-rate [kₒₙ or kₐ] for this association reaction, assuming bimolecular interaction) of equal to or less than 10⁻⁵ M. For example, the equilibrium dissociation constant K_{D} can range from 10⁻⁵ M to 10⁻¹³ M, such as 10⁻⁷ M to 1011 M, 10⁻⁷ M to 10⁻¹⁰ M, 10⁻⁷ M to 10⁻⁹ M, 10⁻⁸ M to 10⁻¹⁰ M, or 10⁻⁹ M to 10⁻¹⁰ M.

The on-rate (association rate constant; kₒₙ or k_{d}; units of 1/Ms or M⁻¹s⁻¹) and the off-rate (dissociation rate constant; k_{off} or k_{d}; units of 1/s or s⁻¹) can be determined using any of the known assay methods, for example, surface plasmon resonance (SPR), or other methods described herein for measuring the binding of one protein to another.

In some embodiments, the binding affinity (EC₅₀) and/or the equilibrium dissociation constant (K_{D}) of the antibody to ROR1, such as human ROR1, is from at or about 0.1 nM to at or about 500 nM, from at or about 0.1 nM to at or about 100 nM, from at or about 0.1 nM to at or about 50 nM, from at or about 0.1 nM to at or about 10 nM, from at or about 0.1 nM to at or about 1 nM, from at or about 1 nM to at or about 500 nM, from at or about 1 nM to at or about 100 nM, from at or about 1 nM to at or about 50 nM, from at or about 1 nM to at or about 10 nM, from at or about 10 nM to at or about 500 nM, from at or about 10 nM to at or about 100 nM, from at or about 10 nM to at or about 50 nM, from at or about 50 nM to at or about 500 nM, from at or about 50 nM to at or about 100 nM or from at or about 100 nM to at or about 500 nM. In certain embodiments, the binding affinity (EC₅₀) and/or the equilibrium dissociation constant (K_{D}) of the antibody to ROR1, such as human ROR1, is at or about or less than at or about 100 nM, 50 nM, 40 nM, 30 nM, 25 nM, 20 nM, 19 nM, 18 nM, 17 nM, 16 nM, 15 nM, 14 nM, 13 nM, 12 nM, 11 nM, 10 nM, 9 nM, 8 nM, 7 nM, 6 nM, 5 nM, 4 nM, 3 nM, 2 nM, or 1 nM, or a range defined by any of the foregoing. In some embodiments, the antibodies bind to ROR1, such as human ROR1, with a sub-nanomolar binding affinity, for example, with a binding affinity less than at or about 1 nM, such as less than at or about 0.9 nM, 0.8 nM, 0.7 nM, 0.6 nM, 0.5 nM, 0.4 nM, 0.3 nM, 0.2 nM or 0.1 nM. In some embodiments, the binding affinity (EC₅₀) and/or the equilibrium dissociation constant, K_{D}, of the binding molecule, e.g., anti-ROR1 antibody or fragment thereof, to a ROR1 protein, such as a human ROR1 protein, is from at or about 0.01 nM to about 1 M, 0.1 nM to 1 M, 1 nM to 1 M, 1 nM to 500 nM, 1 nM to 100 nM, 1 nM to 50 nM, 1 nM to 10 nM, 10 nM to 500 nM, 10 nM to 100 nM, 10 nM to 50 nM, 50 nM to 500 nM, 50 nM to 100 nM or 100 nM to 500 nM. In certain embodiments, the binding affinity (EC₅₀) and/or the equilibrium dissociation constant, K_{D}, of the binding molecule, e.g., anti-ROR1 antibody or fragment thereof, to a ROR1 protein, such as a human ROR1 protein, is at or about or less than at or about 1 M, 500 nM, 100 nM, 50 nM, 40 nM, 30 nM, 25 nM, 20 nM, 19 nM, 18 nM, 17 nM, 16 nM, 15 nM, 14 nM, 13 nM, 12 nM, 11 nM, 10 nM, 9 nM, 8 nM, 7 nM, 6 nM, 5 nM, 4 nM, 3 nM, 2 nM, or 1 nM or less, or a range defined by any of the foregoing.

In some embodiments, the EC₅₀ and/or the K_{D} of the binding molecule, e.g., anti-ROR1 antibody or fragment thereof, to a ROR1 protein, is between at or about 10 nM and at or about 90 nM, between at or about 20 nM and at or about 80 nM, between at or about 30 nM and at or about 70 nM, between at or about 40 nM and at or about 60 nM, or between at or about 40 nM and at or about 50 nM. In certain embodiments, the EC₅₀ and/or the K_{D} of the binding molecule, e.g., anti-ROR1 antibody or fragment thereof, to a ROR1 protein, such as a human ROR1 protein, is at or about 10 nM, 20 nM, 30 nM, 40 nM, 50 nM, 60 nM, 70 nM, 80 nM, 90 nM or 100 nM, or a range defined by any of the foregoing. In certain embodiments, the EC₅₀ and/or the K_{D} of the binding molecule, e.g., anti-ROR1 antibody or fragment thereof, to a ROR1 protein, such as a human ROR1 protein, is at or about 10 nM, 20 nM, 30 nM, 40 nM, 50 nM, 60 nM, 70 nM, 80 nM, 90 nM or 100 nM, or a range defined by any of the foregoing. In certain embodiments, the EC₅₀ and/or the K_{D} of the binding molecule, e.g., anti-ROR1 antibody or fragment thereof, to a ROR1 protein, such as a human ROR1 protein, is at or about 40 nM, 41 nM, 42 nM, 43 nM, 44 nM, or 45 nM, or a range defined by any of the foregoing.

In some embodiments, the V_{H} region of the provided binding molecules comprises a CDR-H1, a CDR-H2 and a CDR-H3 contained within SEQ ID NO:112, and the V_{L} region of the provided binding molecules comprises a CDR-L1, a CDR-L2 and a CDR-L3 contained within SEQ ID: NO 115 and the equilibrium dissociation constant (K_{D}) for human ROR1 is between at or about 30 nM and at or about 50 nM. In some embodiments, the V_{H} region and the V_{L} region of the provided binding molecules are or comprise the sequence set forth in SEQ ID NOS: 112 and 115, respectively, and the off-rate (k_{off} or kₐ) for human ROR1 is at or about 40 nM.

In some embodiments, the provided binding molecule, e.g., anti-ROR1 antibody or antigen-binding fragment thereof or receptors containing such antibody or antigen-binding fragments, has a fast off-rate (dissociation rate constant; k_{off} or k_{d}; units of 1/s or s⁻¹). In some embodiments, the off-rate (k_{off} or kₐ) of the provided binding molecules is between at or about 1×10⁻⁵ s⁻¹ and at or about 1×10⁻² s⁻¹, such as at or about 5×10⁻⁵ s⁻¹ and at or about 9×10⁻³ s⁻¹, at or about 1×10⁻⁴ s⁻¹ and at or about 8×10⁻³ s⁻¹, at or about 5×10⁻⁴ s⁻¹ and at or about 7×10⁻³ s⁻¹, at or about 1×10⁻³ s⁻¹ and at or about 6×10⁻³ s⁻¹, and at or about 4×10⁻³ s⁻¹ and at or about 6×10⁻³ s⁻¹. In some embodiments, the off-rate (k_{off} or k_{d}) of the provided binding molecules is at least at or about 1×10⁻⁵ s⁻¹, 5×10⁻⁵ s⁻¹, 1×10⁻⁴ s⁻¹, 5×10⁻⁴ s⁻¹, 1×10⁻³ s⁻¹, 5×10⁻³ s⁻¹, or 1×10⁻² s⁻¹. In some embodiments, the off-rate (k_{off} or k_{d}) of the provided binding molecules is at least at or about 6×10⁻⁴ s⁻¹, 7×10⁻⁴ s⁻¹, 8×10⁻⁴ s⁻¹, 9×10⁻⁴ s⁻¹, 1×10⁻³ s⁻¹, 2×10⁻³ s⁻¹, 3×10⁻³ s⁻¹, 4×10⁻³ s⁻, 5×10⁻³ s⁻¹, 6×10⁻³ s⁻¹, 7×10³ s⁻¹, 8×10³ s⁻¹, 9×10³ s⁻¹ or 1×10²s⁻¹. In some embodiments, the off-rate (k_{off} or kₐ) of the provided binding molecules is at least at or about 4×10⁻³ s⁻¹, 5×10⁻³ s⁻¹ or 6×10⁻³ s⁻¹, or a range defined by any of the foregoing. In some embodiments, the provided binding molecule, e.g., anti-ROR1 antibody or antigen-binding fragment thereof or receptors containing such antibody or antigen-binding fragments, has an off-rate that is at least at or about 2-fold, 3-fold, 4-fold, 5-fold, 6-fold, 7-fold, 8-fold, 9-fold or 10-fold faster than the off-rate of a reference anti-ROR1 antibody or an antigen-binding fragment thereof, or receptors containing such antibody or antigen-binding fragments, for example, anti-ROR1 antibody R12.

In some embodiments, the V_{H} region of the provided binding molecules comprises a CDR-H1, a CDR-H2 and a CDR-H3 contained within SEQ ID NO:112, and the V_{L} region of the provided binding molecules comprises a CDR-L1, a CDR-L2 and a CDR-L3 contained within SEQ ID: NO 115 and the off-rate (k_{off} or k_{d}) for human ROR1 is between at or about 4×10⁻³ s⁻¹ and at or about 6×10⁻³ s⁻¹. In some embodiments, the V_{H} region and the V_{L} region of the provided binding molecules are or comprise the sequence set forth in SEQ ID NOS: 112 and 115, respectively, and the off-rate (k_{off} or k_{d}) for human ROR1 is at or about 5×10⁻³ s⁻¹.

In some embodiments, the binding affinity of a binding molecule, such as an anti-ROR1 antibody, for different antigens, e.g., ROR1 proteins from different species can be compared to determine the species cross-reactivity. For example, species cross-reactivity can be classified as high cross reactivity or low cross reactivity. In some embodiments, the equilibrium dissociation constant, K_{D}, for different antigens, e.g., ROR1 proteins from different species such as human, cynomolgus monkey or mouse, can be compared to determine species cross-reactivity. In some embodiments, the species cross-reactivity of an anti-ROR1 antibody can be high, e.g., the anti-ROR1 antibody binds to human ROR1 and a species variant ROR1 to a similar degree, e.g., the ratio of K_{D} for human ROR1 and K_{D} for the species variant ROR1 is or is about 1. In some embodiments, the species cross-reactivity of an anti-ROR1 antibody can be low, e.g., the anti-ROR1 antibody has a high affinity for human ROR1 but a low affinity for a species variant ROR1, or vice versa. For example, the ratio of K_{D} for the species variant ROR1 and K_{D} for the human ROR1 is more than 10, 15, 20, 25, 30, 40, 50, 60, 70, 80, 90, 100, 200, 500, 1000, 2000 or more, and the anti-ROR1 antibody has low species cross-reactivity. The degree of species cross-reactivity can be compared with the species cross-reactivity of a known antibody, such as a reference antibody.

In some embodiments, the provided antibodies or antigen binding fragments thereof bind to a similar degree to a human ROR1 protein and a non-human ROR1 protein. For example, in some embodiments, the provided antibodies or antigen binding fragments thereof bind to a human ROR1 protein, or an allelic variant or splice variant thereof, with a specific an equilibrium dissociation constant (K_{D}), and to a non-human ROR1, such as a cynomolgus monkey ROR1, with a K_{D} that is similar, or about the same, or less than 2-fold different, or less than 5-fold different.

For example, in some embodiments, the provided antibodies or antigen binding fragments thereof bind to a human ROR1 with a K_{D} of about or less than at or about 1 M, 500 nM, 100 nM, 50 nM, 40 nM, 30 nM, 25 nM, 20 nM, 19 nM, 18 nM, 17 nM, 16 nM, 15 nM, 14 nM, 13 nM, 12 nM, 11 nM, 10 nM, 9 nM, 8 nM, 7 nM, 6 nM, 5 nM, 4 nM, 3 nM, 2 nM, or 1 nM or less, and binds to a cynomolgus monkey ROR1 with a K_{D} of about or less than at or about 1 μM, 500 nM, 100 nM, 50 nM, 40 nM, 30 nM, 25 nM, 20 nM, 19 nM, 18 nM, 17 nM, 16 nM, 15 nM, 14 nM, 13 nM, 12 nM, 11 nM, 10 nM, 9 nM, 8 nM, 7 nM, 6 nM, 5 nM, 4 nM, 3 nM, 2 nM, or 1 nM or less. In some embodiments, the provided antibodies or antigen binding fragments thereof bind to a mouse ROR1 protein with a K_{D} of about or less than at or about 1 μM, 500 nM, 100 nM, 50 nM, 40 nM, 30 nM, 25 nM, 20 nM, 19 nM, 18 nM, 17 nM, 16 nM, 15 nM, 14 nM, 13 nM, 12 nM, 11 nM, 10 nM, 9 nM, 8 nM, 7 nM, 6 nM, 5 nM, 4 nM, 3 nM, 2 nM, or 1 nM or less. In some embodiments, the provided antibodies or antigen binding fragments thereof bind to a human ROR1, a cynomolgus monkey ROR1 and a mouse ROR1 with high affinity. In some embodiments, the provided antibodies or antigen binding fragments thereof bind to a human ROR1 and cynomolgus monkey ROR1 with a high affinity, and to a mouse ROR1 with low affinity. In some embodiments, the provided antibodies or antigen binding fragments thereof bind to a human ROR1 and ROR1 from other species, or other variants of the ROR1 protein, with high affinity.

In some embodiments, the total binding capacity (Rₘₐₓ), as measured using particular surface plasmon resonance (SPR) conditions, is used to determine the ability or capacity of binding of the provided antibody or antigen binding fragment thereof, to the antigen, e.g., a ROR1 protein, such as a human ROR1 protein. For SPR analysis, the “ligand” is the immobilized target molecule on the surface of the sensor, for example, a ROR1 protein, and the “analyte” is the tested molecule, e.g., antibody, for binding to the “ligand”. For example, the “analyte” can be any of the provided antibodies or antigen binding fragments thereof, that binds to a ROR1 protein. For a particular ligand and analyte pair in SPR, the Rₘₐₓ can be determined assuming a 1:1 binding stoichiometry model, for a particular condition. In some embodiments, binding capacity (Rₘₐₓ) can be determined using the following formula: Rₘₐₓ (RU)=(analyte molecular weight)/(ligand molecular weight)×immobilized ligand level (RU). In particular aspects of SPR conditions, the Rₘₐₓ of binding between any of the provided antibody or antigen binding fragment thereof and a ROR1 protein, such as a human ROR1 or a cynomolgus ROR1, is at least or at least about 50 resonance units (RU), such as about 25 RU, 20 RU, 15 RU, 10 RU, 5 RU or 1 RU.

In some embodiments, properties or features of the provided antibodies are described in relation to properties observed for another antibody, e.g., a reference antibody. In some embodiments, the reference antibody is a non-human anti-ROR1 antibody, such as a rabbit or chimeric or humanized anti-ROR1 antibody. In some aspects, the reference antibody is the chimeric rabbit/human IgG1 antibody designated R12 (see, e.g., Yang et al. (2011) PloS ONE, 6:e21018; U.S. Patent Application No. US 2013/0251642), and/or a fragment derived therefrom such as an scFv fragment thereof, and/or an antibody containing the V_{H} and V_{L} sequences of such an antibody and/or the heavy and light chain CDRs of such an antibody. A chimeric antigen receptor (CAR) containing an antigen-binding scFv fragment of R12 has been demonstrated to effectively promote antitumor reactivity in a CAR therapy (Hudecek et al. (2013) Clin. Cancer Res., 19:3153; International published PCT Appl. No. WO2014031687). In some embodiments, the reference antibody is an scFv that comprises the sequence of amino acids set forth in SEQ ID NO:142.

In some embodiments, the reference antibody is the mouse anti-human ROR1 antibody designated 2A2, and/or a fragment derived therefrom such as an scFv fragment thereof, and/or an antibody containing the V_{H} and V_{L} sequences of such an antibody and/or the heavy and light chain CDRs of such an antibody (see, e.g., Baskar et al. (2012) MAbs, 4:349-361; published U.S. Patent Appl. No. US2012/20058051). For example, in some embodiments, the reference antibody has a V_{H} region containing the sequence set forth in SEQ ID NO: 172 and a V_{L} containing the sequence set forth in SEQ ID NO: 173. In some embodiments, the reference antibody is an scFv form of antibody 2A2.

In some embodiments, the reference antibody is a human or humanized anti-ROR1 antibody. Exemplary humanized anti-ROR1 antibodies are described in International PCT Appl. No. WO2014/031174. In some embodiments, the reference antibody is a humanized variant of an antibody designated 99961. In some embodiments, the reference antibody has a V_{H} region containing the sequence set forth in SEQ ID NO: 174, 175, 176 or 177 and a V_{L} containing the sequence set forth in SEQ ID NO: 178, 179, 180 or 181.

In some embodiments, the provided antibody contains heavy and light chain CDRs that are distinct from the CDRs present in the reference antibody or antibodies. Among the provided antibodies are those that compete for binding with and/or bind to the same or overlapping epitopes of ROR1 as those bound by a reference antibody or antibody, such as R12, but nonetheless contain distinct CDRs, e.g., distinct heavy and/or light chain CDR1, CDR2, and CDR3.

In some embodiments, the antibody has an affinity that is about the same as or lower than that of the corresponding form of the reference antibody, e.g., EC₅₀ or K_{D} that is no more than at or about 1.5-fold or no more than at or about 2-fold greater, no more than at or about 3-fold greater, and/or no more than at or about 10-fold greater, than the EC₅₀ or K_{D} of the corresponding form of the reference antibody. In some embodiments, the antibody has an affinity that is about the same as or lower than that of the corresponding form of the reference antibody, e.g., EC₅₀ or K_{D} that is at least at or about 1.5-fold greater, at least at or about 2-fold greater, at least at or about 3-fold greater, at least at or about 5-fold greater, at least at or about 10-fold greater, at least at or about 20-fold greater, at least at or about 25-fold greater, at least at or about 30-fold greater, at least at or about 40-fold greater, at least at or about 50-fold greater, or at least at or about 100-fold greater, than the EC₅₀ or K_{D} of the corresponding form of the reference antibody. In some embodiments, the antibody has an affinity that is about the same as or lower than that of the corresponding form of the reference antibody, an affinity that is at or about 1.5-fold, 2-fold, 3-fold, 4-fold, 5-fold, 6-fold, 7-fold, 8-fold, 9-fold, 10-fold, 20-fold, 25-fold, 30-fold, 40-fold, 50-fold or 100-fold lower than the affinity of the reference antibody.

In some embodiments, the antibody has an affinity that is greater than that of the corresponding form of the reference antibody, e.g., EC₅₀ or K_{D} that is lower than or lower than at or about 5-fold, 10-fold, 20-fold, 30-fold, 40-fold, 50-fold, 60-fold, 70-fold, 80-fold, 90-fold, 100-fold, 150-fold, 200-fold, 250-fold or lower than the EC₅₀ or K_{D} of the corresponding form of the reference antibody.

In some embodiments, the antibodies display a binding preference for ROR1-expressing cells as compared to ROR1-negative cells, such as particular cells known and/or described herein to express ROR1 and known not to express ROR1, or expressing a related but different antigen, e.g., ROR2. In some embodiments, the binding preference is observed where a significantly greater degree of binding is measured to the ROR1-expressing, as compared to the non-expressing, cells or cells expressing a related but different antigen. In some embodiments, the fold change in degree of binding detected, for example, as measured by mean fluorescence intensity in a flow cytometry-based assay and/or dissociation constant or EC₅₀, to the ROR1-expressing cells as compared to the non-ROR1-expressing cells or cells expressing a related but different antigen, is at least at or about 1.5, 2, 3, 4, 5, 6, or more, and/or is about as great, about the same, at least as great or at least about as great, or greater, than the fold change observed for the corresponding form of the reference antibody. In some cases, the total degree of observed binding to ROR1 or to the ROR1-expressing cells is approximately the same, at least as great, or greater than that observed for the corresponding form of the reference antibody.

In some aspects, the affinity is at or about the same degree or substantially the same degree of affinity compared to the corresponding form of the reference antibody, such as rabbit ROR1 antibody. In some aspects, the affinity is at least at or about 80, 85, 90, 95, or 99% of or the same as that of the corresponding form of the reference antibody.

In some embodiments, the antibody specifically binds to an epitope that overlaps with the epitope of ROR1 bound by a reference antibody. In some aspects, among such antibodies are antibodies that bind to the same or a similar epitope as the reference antibody. In some embodiments, the antibodies bind to the same or a similar epitope or an epitope within the same region or containing residues within the same region of ROR1 as a reference antibody, such as anti-ROR1 antibody R12 or scFv fragment thereof (set forth in SEQ ID NO: 142; see e.g. Yang et al. (2011) PloS ONE, 6:e21018). In some embodiments, the antibody inhibits binding to and/or competes for binding to ROR1, such as human ROR1, with the reference antibody.

Competitive inhibition assays are known and include ELISA-based, flow cytometry-based assays, and RIA-based assays. In some aspects, competitive inhibition assays are carried out by incorporating an excess of an unlabeled form of one of the antibodies and assessing its ability to block binding of the other antibody, which is labeled with a detectable marker, such that degree of binding and reduction thereof can be assessed by detection of the label or marker. In some embodiments, addition of the provided antibody in excess, e.g., 1-, 2-, 5-, 10-, 50- or 100-fold excess, as compared to the amount or concentration of the reference antibody, inhibits binding to the antigen by the reference antibody (or vice versa). In some embodiments, the inhibition of binding is by at least 50%, and in some embodiments by at least 75%, 90% or 99%. In some aspects, the competitive inhibition is as measured in a competitive binding assay (see, e.g., Junghans et al., Cancer Res. 1990:50:1495-1502). Competition assays may be used to identify an antibody that competes with any of the antibodies described herein. Assays for mapping epitopes bound by the antibodies and reference antibodies also may be used and are known.

In some embodiments, where the reference antibody is present at a concentration of or of about 2 nM, the provided antibody inhibits binding of the reference antibody with an IC₅₀ of less than at or about 200 nM, 150 nM, 100 nM, 50 nM, 40 nM, 30 nM, 25 nM, 20 nM, 19 nM, 18 nM, 17 nM, 16 nM, 15 nM, 14 nM, 13 nM, 12 nM, 11 nM, or 10 nM, or less than at or about 9 nM, 8 nM, 7 nM, 6 nM, or 5 nM. In some embodiments, where the provided antibody is present at a concentration of or about 2 nM, the reference antibody inhibits binding of the provided antibody with an IC₅₀ of less than at or about 200 nM, 150 nM, 100 nM, 50 nM, 40 nM, 30 nM, 25 nM, 20 nM, 19 nM, 18 nM, 17 nM, 16 nM, 15 nM, 14 nM, 13 nM, 12 nM, 11 nM, or 10 nM, or less than at or about 9 nM, 8 nM, 7 nM, 6 nM, or 5 nM. In some embodiments, competitive inhibition of the reference antibody's binding by the provided antibody (or vice versa) is at or about or least at or about the same degree as the degree of competitive inhibition of the reference antibody's binding by the reference antibody itself, e.g., unlabeled reference antibody. In some embodiments, the provided antibody inhibits binding of the reference antibody, such as binding of R12 scFv, to human ROR1 by at least 70%, 75%, 80%, 85%, 90%, 91%, 92%, 93%, 94%, 95%, 96%, 97%, 98%, or 99%.

Anti-ROR1 antibodies provided herein may be identified, screened for, or characterized for their physical/chemical properties and/or biological activities by various known assays. In one aspect, the antibody is tested for its antigen binding activity, e.g., by known methods such as ELISA, Western blotting, and/or flow cytometric assays, including cell-based binding assays, for example, assessing binding of the antibody (e.g., conjugated to a fluorescent marker or tagged) to a cell expressing the target antigen, e.g., ROR1, in some cases compared to results using cells that do not express the target antigen, e.g., ROR1, or cells that express a different antigen, e.g., ROR2. Binding affinity may be measured as K_{D}, K_{A} or EC₅₀.

### 3. Variants

In certain embodiments, the antibodies include one or more amino acid variations, e.g., substitutions, deletions, insertions, and/or mutations, compared to the sequence of an antibody described herein. Exemplary variants include those designed to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

In certain embodiments, the antibodies include one or more amino acid substitutions, e.g., as compared to an antibody sequence described herein and/or compared to a sequence of a natural repertoire, e.g., human repertoire. Sites of interest for substitutional mutagenesis include the CDRs and FRs. Amino acid substitutions may be introduced into an antibody of interest and the products screened for a desired activity, e.g., retained/improved antigen binding, decreased immunogenicity, improved half-life, and/or improved effector function, such as the ability to promote antibody-dependent cellular cytotoxicity (ADCC) or complement-dependent cytotoxicity (CDC).

In some embodiments, one or more residues within a CDR of a parent antibody (e.g. a humanized or human antibody) is/are substituted. In some embodiments, the substitution is made to revert a sequence or position in the sequence to a germline sequence, such as an antibody sequence found in the germline (e.g., human germline), for example, to reduce the likelihood of immunogenicity, e.g., upon administration to a human subject.

In some embodiments, alterations are made in CDR “hotspots,” residues encoded by codons that undergo mutation at high frequency during the somatic maturation process (see, e.g., Chowdhury, Methods Mol. Biol. 207:179-196 (2008)), and/or residues that contact antigen, with the resulting variant V_{H} or V_{L} being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, NJ, (2001)). In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves CDR-directed approaches, in which several CDR residues (e.g., 4-6 residues at a time) are randomized. CDR residues involved in antigen binding may be specifically identified, e.g., using alanine scanning mutagenesis or modeling. CDR-H3 and CDR-L3 in particular are often targeted.

In certain embodiments, substitutions, insertions, or deletions may occur within one or more CDRs so long as such alterations do not substantially reduce the ability of the antibody to bind antigen. For example, conservative alterations (e.g., conservative substitutions as provided herein) that do not substantially reduce binding affinity may be made in CDRs. Such alterations may, for example, be outside of antigen contacting residues in the CDRs. In certain embodiments of the variant V_{H} and V_{L} sequences provided above, each CDR either is unaltered, or contains no more than one, two or three amino acid substitutions.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme or a polypeptide which increases the serum half-life of the antibody.

### 4. Modifications

In certain embodiments, the antibody is altered to increase or decrease the extent to which the antibody is glycosylated, for example, by removing or inserting one or more glycosylation sites by altering the amino acid sequence and/or by modifying the oligosaccharide(s) attached to the glycosylation sites, e.g., using certain cell lines.

In some embodiments, an N-linked glycosylation, which is a glycosylation site that occurs at asparagines in the consensus sequence -Asn-Xaa-Ser/Thr is removed or inserted. In some embodiments, one or more re replaced with another amino acid to remove the glycosylation site.

Exemplary modifications, variants, and cell lines are described, e.g., in Patent Publication Nos. US 2003/0157108, US 2004/0093621, US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Ripka et al. Arch. Biochem. Biophys. 249:533-545 (1986); US Pat Appl No US 2003/0157108 A1, Presta, L; and WO 2004/056312 A1, Yamane-Ohnuki et al. Biotech. Bioeng. 87: 614 (2004); Kanda, Y. et al., Biotechnol. Bioeng., 94(4):680-688 (2006); and WO2003/085107); WO 2003/011878 (Jean-Mairet et al.); U.S. Pat. No. 6,602,684 (Umana et al.); and US 2005/0123546 (Umana et al.); WO 1997/30087 (Patel et al.); WO 1998/58964 (Raju, S.); and WO 1999/22764 (Raju, S.).

Among the modified antibodies are those having one or more amino acid modifications in the Fc region, such as those having a human Fc region sequence or other portion of a constant region (e.g., a human IgG1, IgG2, IgG3 or IgG4 Fc region) comprising an amino acid modification (e.g. a substitution) at one or more amino acid positions.

Such modifications can be made, e.g., to improve half-life, alter binding to one or more types of Fc receptors, and/or alter effector functions.

Also among the variants are cysteine engineered antibodies such as “thioMAbs” and other cysteine engineered variants, in which one or more residues of an antibody are substituted with cysteine residues, in order to generate reactive thiol groups at accessible sites, e.g., for use in conjugation of agents and linker-agents, to produce immunoconjugates. Cysteine engineered antibodies are described, e.g., in U.S. Pat. Nos. 7,855,275 and 7,521,541.

In some embodiments, the antibodies are modified to contain additional nonproteinaceous moieties, including water soluble polymers. Exemplary polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer is attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

### B. Immunoconjugates

In some embodiments, the antibody is or is part of an immunoconjugate, in which the antibody is conjugated to one or more heterologous molecule(s), such as, but not limited to, a cytotoxic or an imaging agent. Cytotoxic agents include, but are not limited to, radioactive isotopes (e.g., At²¹¹, I³¹¹, I¹²⁵, Y⁹⁰, Re¹⁸⁶, Re¹⁸⁸, Sm¹⁵³, B1i²¹², P³², Pb²¹² and radioactive isotopes of Lu); chemotherapeutic agents (e.g., methotrexate, adriamicin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins. In some embodiments, the antibody is conjugated to one or more cytotoxic agents, such as chemotherapeutic agents or drugs, growth inhibitory agents, toxins (e.g., protein toxins, enzymatically active toxins of bacterial, fungal, plant, or animal origin, or fragments thereof), or radioactive isotopes.

Among the immunoconjugates are antibody-drug conjugates (ADCs), in which an antibody is conjugated to one or more drugs, including but not limited to a maytansinoid (see U.S. Pat. Nos. 5,208,020, 5,416,064 and European Patent EP 0 425 235 B1); an auristatin such as monomethylauristatin drug moieties DE and DF (MMAE and MMAF) (see U.S. Pat. Nos. 5,635,483 and 5,780,588, and 7,498,298); a dolastatin; a calicheamicin or derivative thereof (see U.S. Pat. Nos. 5,712,374, 5,714,586, 5,739,116, 5,767,285, 5,770,701, 5,770,710, 5,773,001, and 5,877,296; Hinman et al., Cancer Res. 53:3336-3342 (1993); and Lode et al., Cancer Res. 58:2925-2928 (1998)); an anthracycline such as daunomycin or doxorubicin (see Kratz et al., Current Med. Chem. 13:477-523 (2006); Jeffrey et al., Bioorganic & Med. Chem. Letters 16:358-362 (2006); Torgov et al., Bioconj. Chem. 16:717-721 (2005); Nagy et al., Proc. Natl. Acad. Sci. USA 97:829-834 (2000); Dubowchik et al., Bioorg. & Med. Chem. Letters 12:1529-1532 (2002); King et al., J. Med. Chem. 45:4336-4343 (2002); and U.S. Pat. No. 6,630,579); methotrexate; vindesine; a taxane such as docetaxel, paclitaxel, larotaxel, tesetaxel, and ortataxel; a trichothecene; and CC1065.

Also among the immunoconjugates are those in which the antibody is conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes.

Also among the immunoconjugates are those in which the antibody is conjugated to a radioactive atom to form a radioconjugate. Exemplary radioactive isotopes include At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu.

Conjugates of an antibody and cytotoxic agent may be made using any of a number of known protein coupling agents, e.g., linkers, (see Vitetta et al., Science 238:1098 (1987)), WO94/11026. The linker may be a “cleavable linker” facilitating release of a cytotoxic drug in the cell, such as acid-labile linkers, peptidase-sensitive linkers, photolabile linkers, dimethyl linkers, and disulfide-containing linkers (Chari et al., Cancer Res. 52:127-131 (1992); U.S. Pat. No. 5,208,020).

### C. Multispecific Antibodies

In certain embodiments, the ROR1-binding molecules, e.g., antibodies or polypeptides such as chimeric receptors containing the same, are multispecific. Among the multispecific binding molecules are multispecific antibodies, including, e.g. bispecific. Multispecific binding partners, e.g., antibodies, have binding specificities for at least two different sites, which may be in the same or different antigens. In certain embodiments, one of the binding specificities is for ROR1 and the other is for another antigen. In certain embodiments, bispecific antibodies may bind to two different epitopes of ROR1. Bispecific antibodies may also be used to localize cytotoxic agents to cells which express ROR1. Bispecific antibodies can be prepared as full length antibodies or antibody fragments. Among the multispecific antibodies are multispecific single-chain antibodies, e.g., diabodies, triabodies, and tetrabodies, tandem di-scFvs, and tandem tri-scFvs. Also provided are multispecific chimeric receptors, such as multispecific CARs, containing the antibodies. Also provided are multispecific cells containing the antibodies or polypeptides including the same, such as cells containing a cell surface protein including the anti-ROR1 antibody and an additional cell surface protein, such as an additional chimeric receptor, which binds to a different antigen or a different epitope on ROR1.

Exemplary additional antigens include B cell specific antigens, other tumor-specific antigens, such as antigens expressed specifically on or associated with B cell leukemia, lymphoma, B cell chronic lymphocytic leukemia (CLL), acute myeloid leukemia (AML), acute lymphocytic leukemia (ALL), Burkitt's Lymphoma, mantle cell lymphoma (MCL), non-small cell lung cancer (NSCLC), neuroblastoma, renal cell carcinoma, colon cancer, colorectal cancer, breast cancer, epithelial squamous cell cancer, melanoma, myeloma, stomach cancer, brain cancer, lung cancer, pancreatic cancer, cervical cancer, ovarian cancer, liver cancer, bladder cancer, prostate cancer, testicular cancer, thyroid cancer, uterine cancer, adrenal cancer and/or head and neck cancer, and antigens expressed on T cells. Exemplary antigens include CD4, CD5, CD8, CD14, CD15, CD19, CD20, CD21, CD22, CD23, CD25, CD33, CD37, CD38, CD40, CD40L, CD46, CD52, CD54, CD74, CD80, CD126, CD138, B7, MUC-1, Ia, HM1.24, HLA-DR, tenascin, an angiogenesis factor, VEGF, PIGF, ED-B fibronectin, an oncogene, an oncogene product, CD66a-d, necrosis antigens, I1, IL-2, T101, TAC, IL-6, TRAIL-R1 (DR4) and TRAIL-R2 (DR5).

### D. Recombinant Receptors

Among the provided binding molecules, e.g., ROR1 binding molecules, are cell surface proteins, such as recombinant receptors, such as those that include one of the provided antibodies or antigen-binding fragments. Also provided are polynucleotides that encode all or a portion of such cell surface proteins, e.g., receptors. The receptors include antigen receptors and other chimeric receptors that specifically bind to ROR1, such as receptors containing the provided anti-ROR1 antibodies, e.g., antigen-binding fragments. Among the antigen receptors are functional non-TCR antigen receptors, such as chimeric antigen receptors (CARs). The ROR1-binding receptors generally contain antibodies (e.g., antigen-binding fragments), and/or other binding peptides that specifically bind to ROR1, such as to ROR1 proteins, such as a human ROR1 protein. Also provided are cells expressing the recombinant receptors, compositions containing such cells and uses thereof in adoptive cell therapy, such as treatment of diseases and disorders associated with ROR1 expression, compositions and articles of manufacture and uses of the same.

Among the provided polynucleotides are those that encode recombinant receptors, such as antigen receptors, that specifically bind ROR1. In some aspects, the encoded receptors, such as those containing ROR1-binding polypeptides, and compositions and articles of manufacture and uses of the same, also are provided. The provided polynucleotides can be incorporated into constructs, such as deoxyribonucleic acid (DNA) or RNA constructs, such as those that can be introduced into cells for expression of the encoded recombinant ROR1-binding receptors.

### 1. Exemplary Receptors

The provided ROR1-binding cell surface proteins, such as receptors, generally contain an extracellular antigen-binding domain and an intracellular signaling region. Among the provided receptors, e.g., recombinant receptors, are polypeptides containing the provided antibodies or antigen-binding fragment thereof, such as one or more of the provided anti-ROR1 antibody or fragment thereof. In some embodiments, the provided cell surface proteins specifically bind to ROR1, such as a human ROR1.

Among the antigen receptors are chimeric and/or functional non-TCR antigen receptors, such as chimeric antigen receptors (CARs). The chimeric receptors, such as CARs, generally include an extracellular antigen binding domain that includes, is, or is comprises an anti-ROR1 antibody, such as an anti-ROR1 antibody or fragment thereof described herein. In some embodiments, the chimeric receptors, e.g., CARs, include an intracellular signaling domain. In some embodiments, the chimeric receptors also include a spacer and/or a transmembrane domain. In some embodiments, the spacer is located between the extracellular antigen-binding domain and the transmembrane domain. In some embodiments, the CAR contains an extracellular antigen-binding domain, a spacer, a transmembrane region and an intracellular signaling region. Exemplary CARs provided herein include those containing an antigen-binding domain comprising an antibody or antigen-binding fragment thereof described herein, e.g., in Section I.A and/or in Table 2, or those described in Section I.D herein, in Table 3 and/or in Table E1. Also provided are CARs encoded by the polynucleotides described in Section I.E herein, in Table 3 and/or in Table E1.

In some cases, CARs are referred to as first, second, and/or third generation CARs. In some aspects, a first generation CAR is one that solely provides a CD3-chain induced signal upon antigen binding; in some aspects, a second-generation CARs is one that provides such a signal and costimulatory signal, such as one including an intracellular signaling domain from a costimulatory receptor such as CD28 or CD137; in some aspects, a third generation CAR in some aspects is one that includes multiple costimulatory domains of different costimulatory receptors.

In some embodiments, the CAR contains an extracellular antigen-binding domain, in some cases comprising an antibody, e.g., an antibody fragment that binds ROR1, a transmembrane domain that is or contains a transmembrane portion of CD28 or a functional variant thereof, and an intracellular signaling domain containing a signaling portion of CD28 or functional variant thereof and a signaling portion of CD3 zeta or functional variant thereof. In some embodiments, the CAR contains an extracellular antigen-binding domain, in some cases comprising an antibody, e.g., antibody fragment that binds ROR1, a transmembrane domain that is or contains a transmembrane portion of CD28 or a functional variant thereof, and an intracellular signaling domain containing a signaling portion of a 4-1BB or functional variant thereof and a signaling portion of CD3 zeta or functional variant thereof. In some such embodiments, the receptor further includes a spacer containing a portion of an Ig molecule, such as a human Ig molecule, such as an Ig hinge, e.g., an IgG4 hinge, such as a hinge-only spacer.

In some embodiments, the chimeric antigen receptor includes an extracellular portion containing the antibody or fragment thereof described herein. In some aspects, the chimeric antigen receptor includes an extracellular portion containing the antibody or fragment thereof described herein and an intracellular signaling domain. In some embodiments, the antibody or fragment thereof includes an scFv and the intracellular signaling region contains an ITAM. In some aspects, the intracellular signaling domain includes a signaling domain of a zeta chain of a CD3-zeta (CD3ζ) chain. In some embodiments, the chimeric antigen receptor includes a transmembrane domain linking the extracellular domain and the intracellular signaling domain.

Other exemplary antigen receptors, including CARs, and methods for engineering and introducing such receptors into cells, include those described, for example, in WO200014257, WO2013126726, WO2012/129514, WO2014031687, WO2013/166321, WO2013/071154, WO2013/123061 U.S. patent application publication numbers US2002131960, US2013287748, US20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, and 8,479,118, and European patent application number EP2537416, and/or those described by Sadelain et al., Cancer Discov. 2013 April; 3(4): 388-398; Davila et al. (2013) PLoS ONE 8(4): e61338; Turtle et al., Curr. Opin. Immunol., 2012 October; 24(5): 633-39; Wu et al., Cancer, 2012 Mar. 18(2): 160-75. In some aspects, the antigen receptors include a CAR as described in U.S. Pat. No. 7,446,190, and those described in WO/2014055668 A1. Exemplary of the CARs include CARs as disclosed in any of the aforementioned publications, such as WO2014031687, U.S. Pat. Nos. 8,339,645, 7,446,179, US 2013/0149337, U.S. Pat. Nos. 7,446,190, 8,389,282, e.g., and in which the antigen-binding portion, e.g., scFv, is replaced by an antibody, e.g., as provided herein.

Other ROR1-targeting CARs are described, for example, by Hudecek et al., Clin Cancer Res, 19(12), 3153-3164 (2013) and Baskar et al. MAbs. 4(3): 349-361 (2012). See also WO2014031687; US2012/20058051.

a. Extracellular Antigen-Binding Domain

Among the chimeric receptors are chimeric antigen receptors (CARs). The chimeric receptors, such as CARs, generally include an extracellular antigen binding domain that includes, is, or is comprised within, one or more of the provided anti-ROR1 antibodies or antigen-binding fragments. Thus, the chimeric receptors, e.g., CARs, typically include in their extracellular portions one or more ROR1-binding molecules, such as one or more antigen-binding fragment, domain, or portion, or one or more antibody variable domains and/or antibody molecules, such as those described herein. In some embodiments, the CAR includes a ROR1-binding portion or portions of the antibody molecule, such as a variable heavy (V_{H}) chain region and/or variable light (V_{L}) chain region of the antibody, e.g., an scFv. In some embodiments, the CAR includes a ROR1-binding portion or portions of the antibody molecule, such as a variable heavy (V_{H}) chain region and a variable light (V_{L}) chain region of the antibody, e.g., an scFv. In some aspects, the CAR includes one or more of any of the ROR1-binding antibodies or antigen-binding fragments thereof described herein, e.g., in Section I.A.

In some embodiments, Table 2 provides the SEQ ID NOS: of exemplary antigen-binding domains, such as antibodies or antigen-binding fragments, that can be comprised in the provided ROR1-binding receptors, such as anti-ROR1 chimeric antigen receptors (CARs). In some aspects, the CAR comprises an scFv described in Table 2, for example, as a part of the extracellular antigen-binding domain. In some aspects, the CAR comprises a V_{H} region described in Table 2, for example, as a part of the extracellular antigen-binding domain. In some aspects, the CAR comprises a V_{L} region described in Table 2, for example, as a part of the extracellular antigen-binding domain. In some embodiments, the ROR1-binding receptor contains a ROR1-binding antibody or fragment thereof, comprising a V_{H} region that comprises a CDR-H1, a CDR-H2 and a CDR-H3 sequence and a V_{L} region that comprises a CDR-L1, a CDR-L2 and a CDR-L3 sequence set forth in the SEQ ID NOS: listed in each row of Table 2. In some embodiments, the ROR1-binding receptor contains a ROR1-binding antibody or fragment thereof, comprising a V_{H} region sequence and a V_{L} region sequence set forth in the SEQ ID NOS: listed in each row of Table 2, or an antibody comprising a V_{H} and V_{L} region amino acid sequence that has at least 90%, 91%, 92%, 93%, 94%, 95%, 96%, 97%, 98%, or 99% sequence identity to the V_{H} region sequence and the V_{L} region sequence set forth in the SEQ ID NOS: listed in each row of Table 2. In some embodiments, the ROR1-binding receptor contains a ROR1-binding antibody or fragment thereof, comprising a V_{H} region sequence and a V_{L} region sequence set forth in the SEQ ID NOS: listed in each row of Table 2. In some embodiments, the ROR1-binding receptor contains a ROR1-binding antibody or fragment thereof, comprising an scFv sequence set forth in the SEQ ID NOS: listed in each row of Table 2, or an antibody comprising an scFv amino acid sequence that has at least 90%, 91%, 92%, 93%, 94%, 95%, 96%, 97%, 98%, or 99% sequence identity to the scFv sequence set forth in the SEQ ID NOS: listed in each row of Table 2. In some embodiments, the ROR1-binding receptor contains a ROR1-binding antibody or fragment thereof, comprising an scFv sequence set forth in the SEQ ID NOS: listed in each row of Table 2.

In some embodiments, the provided CARs can include an extracellular antigen-binding domain that contains all or a portion of an antibody, including polyclonal and monoclonal antibodies, including intact antibodies and functional (antigen-binding) antibody fragments, including fragment antigen binding (Fab) fragments, F(ab′)₂ fragments, Fab′ fragments, Fv fragments, recombinant IgG (rIgG) fragments, heavy chain variable (V_{H}) regions capable of specifically binding the antigen, single chain antibody fragments, including single chain variable fragments (scFv), and single domain antibodies (e.g., sdAb, sdFv, nanobody) fragments. In some aspects, the antibody or fragment thereof contained in the CARs include genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific or trispecific, antibodies, diabodies, triabodies, and tetrabodies, tandem di-scFv, tandem tri-scFv.

b. Spacer

In some embodiments, the recombinant receptor such as the CAR, such as the antibody portion thereof, further includes a spacer (in some cases also called a spacer region), which may be or include at least a portion of an immunoglobulin constant region or variant or modified version thereof, such as a hinge region, e.g., an IgG4 hinge region, and/or a C_{H}1/C_{L}, C_{H}2 and/or C_{H}3 and/or Fc region. In some embodiments, the constant region or portion is of a human IgG, such as IgG4, IgG2 or IgG1. In some aspects, the portion of the constant region serves as a spacer between the antigen-recognition component, e.g., scFv, and transmembrane domain.

In some embodiments, the length of the spacer is adjusted to optimize the biophysical synapse distance between the CAR-expressing cell, such as a CAR-expressing cell, and the target of the CAR, such as a ROR1-expressing tumor cell. In some embodiments, the CAR is expressed by a T cell, and the length of the spacer is adjusted to a length that is compatible for T cell activation or to optimize CAR T-cell performance.

In some embodiments, the spacer can be of a length that provides for increased responsiveness of the cell following antigen binding, as compared to in the absence of the spacer or as compared to an alternative spacer of a different length (e.g. longer in length). In some examples, the spacer is at or about 12 amino acids in length or is no more than at or about 12 amino acids in length. In some examples, the spacer is at or about 15 amino acids in length or is no more than at or about 15 amino acids in length.

Exemplary spacers include those having at least at or about 10 to at or about 300 amino acids, at or about 10 to at or about 229 amino acids, at or about 10 to at or about 200 amino acids, at or about 10 to at or about 175 amino acids, at or about 10 to at or about 150 amino acids, at or about 10 to at or about 125 amino acids, at or about 10 to at or about 100 amino acids, at or about 10 to at or about 75 amino acids, at or about 10 to at or about 50 amino acids, at or about 10 to at or about 40 amino acids, at or about 10 to at or about 30 amino acids, at or about 10 to at or about 20 amino acids, or at or about 10 to at or about 15 amino acids in length, and including any integer between the endpoints of any of the listed ranges. Exemplary spacers include those having at least at or about at or about 50 to at or about 175 amino acids, at or about 50 to at or about 150 amino acids, at or about 10 to at or about 125 amino acids, at or about 50 to at or about 100 amino acids, at or about 100 to at or about 300 amino acids, at or about 100 to at or about 250 amino acids, at or about 125 to at or about 250 amino acids, or at or about 200 to at or about 250 amino acids, and including any integer between the endpoints of any of the listed ranges. In some embodiments, a spacer is at least at or about 12 amino acids, at least at or about 119 amino acids, at least at or about 125 amino acids, at least at or about 200 amino acids, or at least at or about 220 amino acids, or at least at or about 225 amino acids in length. In some embodiments, a spacer is at least at or about 13 amino acids, at least at or about 120 amino acids, at least at or about 125 amino acids, at least at or about 200 amino acids, or at least at or about 220 amino acids, or at least at or about 229 amino acids in length. In some embodiments, a spacer is at or about 11, 12, 13, 14, 15, 16, 17, 18, 19, 20, 21, 22, 23, 24, 25 amino acids or less in length. In some embodiments, the spacer is at least at or about 100 amino acids in length, such as at least at or about 110, 111, 112, 113, 114, 115, 116, 117, 118, 119, 120, 121, 122, 123, 124, 125, 130, 135, 140, 145, 150, 160, 170, 180, 190, 200, 210, 220, 230, 240, or 250 amino acids in length.

In some embodiments, the spacer is at least at or about 125 to at or about 300 amino acids, at or about 125 to at or about 250 amino acids, at or about 125 to at or about 230 amino acids, at or about 125 to at or about 200 amino acids, at or about 125 to at or about 180 amino acids, at or about 125 to at or about 150 amino acids, at or about 150 to at or about 300 amino acids, at or about 150 to at or about 250 amino acids, at or about 150 to at or about 230 amino acids, at or about 150 to at or about 200 amino acids, at or about 150 to at or about 180 amino acids, at or about 180 to at or about 300 amino acids, at or about 180 to at or about 250 amino acids, at or about 180 to at or about 230 amino acids, at or about 180 to at or about 200 amino acids, at or about 200 to at or about 300 amino acids, at or about 200 to at or about 250 amino acids, at or about 200 to at or about 230 amino acids, at or about 230 to at or about 300 amino acids, at or about 230 to at or about 250 amino acids in length or 250 to at or about 300 amino acids in length. In some embodiments, the spacer is at least at or about 129, 130, 140, 150, 160, 170, 180, 190, 200, 210, 220, 221, 222, 223, 224, 225, 226, 227, 228, 229 or 230 amino acids in length, or a length between any of the foregoing.

Exemplary spacers include an IgG hinge alone, an IgG hinge linked to one or more of a C_{H}2 and C_{H}3 domain, or IgG hinge linked to the C_{H}3 domain. In some embodiments, the spacer includes an IgG hinge alone. In some embodiments, the IgG hinge, C_{H}2 and/or C_{H}3 can be derived all or in part from IgG4 or IgG2, such as all or in part from human IgG4 or human IgG2. In some embodiments, the spacer can be a chimeric polypeptide containing one or more of a hinge, C_{H}2 and/or C_{H}3 sequence(s) derived from IgG4, IgG2, and/or IgG2 and IgG4. In some embodiments, the hinge region comprises all or a portion of an IgG4 hinge region. In some embodiments, the hinge region comprises all or a portion of an IgG4 hinge region and/or of an IgG2 hinge region, wherein the IgG4 hinge region is optionally a human IgG4 hinge region and the IgG2 hinge region is optionally a human IgG2 hinge region; the C_{H}2 region comprises all or a portion of an IgG4 C_{H}2 region and/or of an IgG2 C_{H}2 region, wherein the IgG4 C_{H}2 region is optionally a human IgG4 C_{H}2 region and the IgG2 C_{H}2 region is optionally a human IgG2 C_{H}2 region; and/or the C_{H}3 region comprises all or a portion of an IgG4 C_{H}3 region and/or of an IgG2 C_{H}3 region, wherein the IgG4 C_{H}3 region is optionally a human IgG4 C_{H}3 region and the IgG2 C_{H}3 region is optionally a human IgG2 C_{H}3 region. In some embodiments, the hinge, C_{H}2 and C_{H}3 comprises all or a portion of each of a hinge region, C_{H}2 and C_{H}3 from IgG4. In some embodiments, the hinge region is chimeric and comprises a hinge region from human IgG4 and human IgG2; the C_{H}2 region is chimeric and comprises a C_{H}2 region from human IgG4 and human IgG2; and/or the C_{H}3 region is chimeric and comprises a C_{H}3 region from human IgG4 and human IgG2. In some embodiments, the spacer comprises an IgG4/2 chimeric hinge or a modified IgG4 hinge comprising at least one amino acid replacement compared to human IgG4 hinge region; an human IgG2/4 chimeric C_{H}2 region; and a human IgG4 C_{H}3 region.

In some embodiments, the spacer comprises or consists of all or a portion of an immunoglobulin hinge or a modified version thereof. In some embodiments, the spacer is at or about 15 amino acids or less in length. In some embodiments, the spacer comprises or consists of all or a portion of an immunoglobulin hinge, optionally an IgG4 hinge, or a modified version thereof and/or comprises about 15 amino acids or less. In some embodiments, the spacer is at or about 13 amino acids in length and/or comprises or consists of all or a portion of an immunoglobulin hinge, optionally an IgG4, or a modified version thereof. In some embodiments, the spacer is at or about 12 amino acids in length and/or comprises or consists of all or a portion of an immunoglobulin hinge, optionally an IgG4, or a modified version thereof. In some embodiments, the spacer comprises or consists of the sequence of SEQ ID NO: 1, 26, 27, 28, 29, 31, 32, 33 or 135, or a variant of any of the foregoing having at least 85%, 86%, 87%, 88%, 89%, 90%, 91%, 92%, 93%, 94%, 95%, 96%, 97%, 98%, 99% or more sequence identity thereto. In some embodiments, the spacer comprises the formula X₁PPX₂P (SEQ ID NO:25), where X₁ is glycine, cysteine or arginine and X₂ is cysteine or threonine. In some embodiments, the spacer does not comprise a CD28 extracellular region or a CD8 extracellular region. In certain cases, the spacer has a methionine residue at the C-terminus.

In some of any such embodiments, the spacer is or contains the sequence set forth in SEQ ID NO:1. In some of any such embodiments, the spacer is or contains the amino acid sequence encoded by SEQ ID NO: 2 or 30 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 2 or 30. In some of any such embodiments, the spacer is or contains the amino acid sequence encoded by SEQ ID NO: 30.

In some of any such embodiments, the spacer is or contains the sequence set forth in SEQ ID NO:135. In some of any such embodiments, the spacer is or contains the amino acid sequence encoded by SEQ ID NO: 192 or 136 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 192 or 136. In some of any such embodiments, the spacer is or contains the amino acid sequence encoded by SEQ ID NO: 136.

In some embodiments, the spacer is or comprises IgG hinge linked to the C_{H}3 domain, e.g., of a human immunoglobulin, such as IgG4 and/or IgG2. In some aspects, the spacer is at or about 111, 112, 113, 114, 115, 116, 117, 118, 119, 120, 121, 122, 123, 124 or 125 amino acids in length, or has a length between any of the foregoing. In some aspects, the spacer is at or about 119 or 120 amino acids in length. In some embodiments, the spacer comprises or consists of the sequence of SEQ ID NO: 3 or 138, or a variant of any of the foregoing having at least 85%, 86%, 87%, 88%, 89%, 90%, 91%, 92%, 93%, 94%, 95%, 96%, 97%, 98%, 99% or more sequence identity thereto.

In some of any such embodiments, the spacer is or contains an amino acid sequence having at least at or about 85%, 86%, 87%, 88%, 89%, 90%, 91%, 92%, 93%, 94%, 95%, 96%, 97%, 98% or 99% identity to SEQ ID NO: 138. In some of any such embodiments, the spacer is or contains the sequence set forth in SEQ ID NO:138. In some of any such embodiments, the spacer is or contains the amino acid sequence encoded by SEQ ID NO: 193 or 139 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 193 or 139. In some of any such embodiments, the spacer is or contains the amino acid sequence encoded by SEQ ID NO: 139.

In some of any such embodiments, the spacer is at or about 120 amino acids in length. In some of any such embodiments, the spacer is or contains an amino acid sequence having at least at or about 85%, 86%, 87%, 88%, 89%, 90%, 91%, 92%, 93%, 94%, 95%, 96%, 97%, 98% or 99% identity to SEQ ID NO: 3. In some of any such embodiments, the spacer is or contains the sequence set forth in SEQ ID NO:3. In some of any such embodiments, the spacer is or contains the amino acid sequence encoded by SEQ ID NO: 4 or 137 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 4 or 137. In some of any such embodiments, the spacer is or contains the amino acid sequence encoded by SEQ ID NO: 137.

In some embodiments, the spacer can be from all or in part from IgG4 and/or IgG2 and can contain mutations, such as one or more single amino acid mutations in one or more domains. In some examples, the amino acid modification is a substitution of a proline (P) for a serine (S) in the hinge region of an IgG4. In some embodiments, the amino acid modification is a substitution of a glutamine (Q) for an asparagine (N) to reduce glycosylation heterogeneity, such as an N177Q mutation at position 177, in the C_{H}2 region, of the full-length IgG4 Fe sequence set forth in SEQ ID NO: 48 or an N176Q at position 176, in the C_{H}2 region, of the full-length IgG2 Fe sequence set forth in SEQ ID NO:49. In some embodiments, the spacer is or comprises an IgG4/2 chimeric hinge or a modified IgG4 hinge; an IgG2/4 chimeric C_{H}2 region; and an IgG4 C_{H}3 region and optionally is about 228 or 229 amino acids in length; or a spacer set forth in SEQ ID NO: 37 or 194.

In some of any such embodiments, the spacer is or contains an amino acid sequence having at least at or about 85%, 86%, 87%, 88%, 89%, 90%, 91%, 92%, 93%, 94%, 95%, 96%, 97%, 98% or 99% identity to SEQ ID NO:194. In some of any such embodiments, the spacer is or contains the sequence set forth in SEQ ID NO: 194. In some of any such embodiments, the spacer is or contains the amino acid sequence encoded by SEQ ID NO: 195 or 196 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 195 or 196. In some of any such embodiments, the spacer is or contains the amino acid sequence encoded by SEQ ID NO: 196.

In some of any such embodiments, the spacer is or contains an amino acid sequence having at least at or about 85%, 86%, 87%, 88%, 89%, 90%, 91%, 92%, 93%, 94%, 95%, 96%, 97%, 98% or 99% identity to SEQ ID NO:37. In some of any such embodiments, the spacer is or contains the sequence set forth in SEQ ID NO: 37. In some of any such embodiments, the spacer is or contains the amino acid sequence encoded by SEQ ID NO: 38 or 140 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 38 or 140. In some of any such embodiments, the spacer is or contains the amino acid sequence encoded by SEQ ID NO: 140.

In some embodiments, the spacer is encoded by a polynucleotide that has been optimized for codon expression and/or to eliminate splice sites such as cryptic splice sites. In some embodiments, the coding sequence for the spacer comprises the nucleic acid sequence set forth in SEQ ID NO: 30. In some embodiments, the coding sequence for the spacer comprises the nucleic acid sequence set forth in SEQ ID NO: 136. In some embodiments, the coding sequence for the spacer comprises the nucleic acid sequence set forth in SEQ ID NO: 137. In some embodiments, the coding sequence for the spacer comprises the nucleic acid sequence set forth in SEQ ID NO: 139. In some embodiments, the coding sequence for the spacer comprises the nucleic acid sequence set forth in SEQ ID NO: 140. In some embodiments, the coding sequence for the spacer comprises the nucleic acid sequence set forth in SEQ ID NO: 196.

Additional exemplary spacers include, but are not limited to, those described in Hudecek et al. (2013) Clin. Cancer Res., 19:3153, Hudecek et al. (2015) Cancer Immunol. Res., 3(2):125-135, or WO2014031687. In some embodiments, the nucleotide sequence of the spacer is optimized to reduce RNA heterogeneity upon expression. In some embodiments, the nucleotide sequence of the spacer is optimized to reduce cryptic splice sites or reduce the likelihood of a splice event at a splice site.

In some embodiments, the spacer has the amino acid sequence set forth in SEQ ID NO:1, and is encoded by the polynucleotide sequence set forth in SEQ ID NO:2. In some embodiments, the spacer has the amino acid sequence set forth in SEQ ID NO:135, and is encoded by the polynucleotide sequence set forth in SEQ ID NO:192. In some embodiments, the spacer has the amino acid sequence set forth in SEQ ID NO:3, and is encoded by the polynucleotide sequence set forth in SEQ ID NO:4. In some embodiments, the spacer has the amino acid sequence set forth in SEQ ID NO:138, and is encoded by the polynucleotide sequence set forth in SEQ ID NO:193. In some embodiments, the spacer has the amino acid sequence set forth in SEQ ID NO:37, and is encoded by the polynucleotide sequence set forth in SEQ ID NO:38. In some embodiments, the spacer has the amino acid sequence set forth in SEQ ID NO:194, and is encoded by the polynucleotide sequence set forth in SEQ ID NO:195.

In some embodiments, the spacer is encoded by a polynucleotide that has been optionally optimized for codon usage and/or to reduce RNA heterogeneity, e.g., by removing cryptic splice sites. In some embodiments, the spacer has the amino acid sequence set forth in SEQ ID NO:1, and is encoded by the polynucleotide sequence set forth in SEQ ID NO:30. In some embodiments, the spacer has the amino acid sequence set forth in SEQ ID NO:135, and is encoded by the polynucleotide sequence set forth in SEQ ID NO:136. In some embodiments, the spacer has the amino acid sequence set forth in SEQ ID NO:3, and is encoded by the polynucleotide sequence set forth in SEQ ID NO:137. In some embodiments, the spacer has the amino acid sequence set forth in SEQ ID NO:138, and is encoded by the polynucleotide sequence set forth in SEQ ID NO:139. In some embodiments, the spacer has the amino acid sequence set forth in SEQ ID NO:37, and is encoded by the polynucleotide sequence set forth in SEQ ID NO:140. In some embodiments, the spacer has the amino acid sequence set forth in SEQ ID NO:194, and is encoded by the polynucleotide sequence set forth in SEQ ID NO:196. In some embodiments, the spacer has an amino acid sequence that exhibits at least at or about 85%, 86%, 87%, 88%, 89%, 90%, 91%, 92%, 93%, 94%, 95%, 96%, 97%, 98% or 99% sequence identity to SEQ ID NO:1, 3 or 37 and is encoded by a polynucleotide that has been optionally optimized for codon usage and/or to reduce RNA heterogeneity. In some embodiments, the spacer has an amino acid sequence that exhibits at least at or about 85%, 86%, 87%, 88%, 89%, 90%, 91%, 92%, 93%, 94%, 95%, 96%, 97%, 98% or 99% sequence identity to SEQ ID NO:135, 138 or 194 and is encoded by a polynucleotide that has been optionally optimized for codon usage and/or to reduce RNA heterogeneity.

c. Transmembrane Domain

The antigen-recognition component generally is linked to one or more intracellular signaling components, such as signaling components that mimic activation through an antigen receptor complex, such as a TCR complex, in the case of a CAR, and/or signal via another cell surface receptor. Thus, in some embodiments, a ROR1-binding molecule (e.g., antibody or antigen binding fragment thereof) is linked to one or more transmembrane domains such as those described herein and intracellular signaling domains comprising one or more intracellular components such as those described herein. In some embodiments, the transmembrane domain is fused to the extracellular domain. In one embodiment, a transmembrane domain that naturally is associated with one of the domains in the receptor, e.g., CAR, is used. In some instances, the transmembrane domain is selected or modified by amino acid substitution to avoid binding of such domains to the transmembrane domains of the same or different surface membrane proteins to minimize interactions with other members of the receptor complex.

The transmembrane domain in some embodiments is derived either from a natural or from a synthetic source. Where the source is natural, the domain in some aspects is derived from any membrane-bound or transmembrane protein. Transmembrane domains include those derived from (i.e. comprise at least the transmembrane domain(s) of) the alpha, beta or zeta chain of the T-cell receptor, CD3 epsilon, CD4, CD5, CD8, CD9, CD16, CD22, CD28, CD33, CD37, CD45, CD64, CD80, CD86, CD134, CD137, and/or CD154. For example, the transmembrane domain can be a CD28 transmembrane domain that comprises the sequence of amino acids set forth in SEQ ID NO: 149, encoded by the nucleic acid sequence set forth in SEQ ID NO:147 or 148. For example, the transmembrane domain can be a CD28 transmembrane domain that comprises the sequence of amino acids set forth in SEQ ID NO: 8, encoded by the nucleic acid sequence set forth in SEQ ID NO:197 or 198. In some embodiments, the transmembrane domain of the receptor, e.g., the CAR is a transmembrane domain of human CD28 or variant thereof, e.g., a 27-amino acid transmembrane domain of a human CD28 (Accession No.: P10747.1), or a 28-amino acid sequence, or is a transmembrane domain that comprises the sequence of amino acids set forth in SEQ ID NO:8 or 149 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 8 or 149. In some embodiments, the transmembrane domain is encoded by a polynucleotide that has been optionally optimized for codon usage and/or to reduce RNA heterogeneity, e.g., by removing cryptic splice sites. In some embodiments, the transmembrane domain has the amino acid sequence set forth in SEQ ID NO:8 or 149, and is encoded by the polynucleotide sequence set forth in SEQ ID NO:147, 148, 197 or 198, or a polynucleotide sequence having at least 90% sequence identity thereto. In certain cases, the transmembrane domain has a methionine residue at the N-terminus.

In some of any such embodiments, the transmembrane domain is or contains SEQ ID NO: 8 or an amino acid sequence having at least at or about 90%, 91%, 92%, 93%, 94%, 95%, 96%, 97%, 98% or 99% sequence identity to SEQ ID NO: 8. In some of any such embodiments, the transmembrane domain is or contains the sequence set forth in SEQ ID NO: 8. In some of any such embodiments, the transmembrane domain is or contains the amino acid sequence encoded by SEQ ID NO: 197 or 198 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 197 or 198. In some of any such embodiments, the transmembrane domain is or contains the amino acid sequence encoded by SEQ ID NO: 198.

In some of any such embodiments, the transmembrane domain is or contains SEQ ID NO: 149 or an amino acid sequence having at least at or about 90%, 91%, 92%, 93%, 94%, 95%, 96%, 97%, 98% or 99% sequence identity to SEQ ID NO: 149. In some of any such embodiments, the transmembrane domain is or contains the sequence set forth in SEQ ID NO: 149. In some of any such embodiments, the transmembrane domain is or contains the amino acid sequence encoded by SEQ ID NO: 147 or 148 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 147 or 148. In some of any such embodiments, the transmembrane domain is or contains the amino acid sequence encoded by SEQ ID NO: 148.

Alternatively, the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain. In some embodiments, the linkage is by linkers, spacers, and/or transmembrane domain(s).

d. Intracellular Signaling Components

Among the intracellular signaling domains are those that mimic or approximate a signal through a natural antigen receptor, a signal through such a receptor in combination with a costimulatory receptor, and/or a signal through a costimulatory receptor alone. In some embodiments, a short oligo- or polypeptide linker, for example, a linker of between 2 and 10 amino acids in length, such as one containing glycines and serines, e.g., glycine-serine doublet, is present and forms a linkage between the transmembrane domain and the intracellular signaling domain of the CAR.

The receptor, e.g., the CAR, generally includes an intracellular signaling region comprising at least one intracellular signaling component or components. In some embodiments, the receptor includes an intracellular component or signaling domain of a TCR complex, such as a TCR CD3 chain that mediates T-cell activation and cytotoxicity, e.g., CD3 zeta (CD3-Q) chain. Thus, in some aspects, the ROR1-binding antibody is linked to one or more cell signaling modules. In some embodiments, cell signaling modules include CD3 transmembrane domain, CD3 intracellular signaling domains, and/or other CD transmembrane domains. In some embodiments, the receptor, e.g., CAR, further includes a portion of one or more additional molecules such as Fc receptor γ, CD8, CD4, CD25, or CD16. For example, in some aspects, the CAR includes a chimeric molecule between CD3-zeta (CD3-ζ) or Fc receptor γ and CD8, CD4, CD25 or CD16.

In some embodiments, upon ligation of the CAR, the cytoplasmic domain or intracellular signaling region of the CAR stimulates and/or activates at least one of the normal effector functions or responses of the immune cell, e.g., T cell engineered to express the CAR. For example, in some contexts, the CAR induces a function of a T cell such as cytolytic activity or T-helper activity, such as secretion of cytokines or other factors. In some embodiments, a truncated portion of an intracellular signaling domain of an antigen receptor component or costimulatory molecule is used in place of an intact immunostimulatory chain, for example, if it transduces the effector function signal. In some embodiments, the intracellular signaling domain or domains include the cytoplasmic sequences of the T cell receptor (TCR), and in some aspects also those of co-receptors that in the natural context act in concert with such receptor to initiate signal transduction following antigen receptor engagement, and/or any derivative or variant of such molecules, and/or any synthetic sequence that has the same functional capability.

In the context of a natural TCR, full activation generally requires not only signaling through the TCR, but also a costimulatory signal. Thus, in some embodiments, to promote full activation, a component for generating secondary or co-stimulatory signal is also included in the CAR. In other embodiments, the CAR does not include a component for generating a costimulatory signal. In some aspects, an additional CAR is expressed in the same cell and provides the component for generating the secondary or costimulatory signal.

T cell activation is in some aspects described as being mediated by two classes of cytoplasmic signaling sequences: those that initiate antigen-dependent primary activation through the TCR (primary cytoplasmic signaling sequences), and those that act in an antigen-independent manner to provide a secondary or co-stimulatory signal (secondary cytoplasmic signaling sequences). In some aspects, the CAR includes one or both of such classes of cytoplasmic signaling sequences.

In some aspects, the CAR includes a primary cytoplasmic signaling sequence that regulates primary stimulation and/or activation of the TCR complex. Primary cytoplasmic signaling sequences that act in a stimulatory manner may contain signaling motifs which are known as immunoreceptor tyrosine-based activation motifs or ITAMs. Examples of ITAM containing primary cytoplasmic signaling sequences include those derived from TCR or CD3 zeta, FcR gamma, CD3 gamma, CD3 delta and CD3 epsilon. In some embodiments, the intracellular signaling region in the CAR contain(s) a cytoplasmic signaling domain, portion thereof, or sequence derived from CD3 zeta. In some embodiments the CD3 zeta comprises the sequence of amino acids set forth in SEQ ID NO:13, 14 or 15, encoded by the nucleic acid sequence set forth in SEQ ID NO: 150 or 182. In some embodiments, the CD3 zeta is encoded by a polynucleotide that has been optionally optimized for codon usage and/or to reduce RNA heterogeneity, e.g., by removing cryptic splice sites. In some embodiments, the CD3 zeta has the amino acid sequence set forth in SEQ ID NO:13, and is encoded by the polynucleotide sequence set forth in SEQ ID NO:150. In some embodiments, the intracellular signaling domain comprises a human CD3 zeta stimulatory signaling domain or functional variant thereof, such as an 112 AA cytoplasmic domain of isoform 3 of human CD3ζ (Accession No.: P20963.2) or a CD3 zeta signaling domain as described in U.S. Pat. No. 7,446,190 or U.S. Pat. No. 8,911,993. In some embodiments, the intracellular signaling domain comprises the sequence of amino acids set forth in SEQ ID NO: 13, 14 or 15 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 13, 14 or 15.

In some embodiments, the CAR includes a signaling domain (e.g., an intracellular or cytoplasmic signaling domain) and/or transmembrane portion of a costimulatory molecule, such as a T cell costimulatory molecule. Exemplary costimulatory molecules include CD28, 4-1BB, OX40, DAP10, and ICOS. For example, a costimulatory molecule can be derived from 4-1BB and can comprise the amino acid sequence set forth in SEQ ID NO: 12, encoded by the nucleotide sequence set forth in SEQ ID NO: 154 or 155. In some embodiments, the costimulatory molecule from 4-1BB is encoded by a polynucleotide that has been optionally optimized for codon usage and/or to reduce RNA heterogeneity, e.g., by removing cryptic splice sites. In some embodiments, the costimulatory molecule from 4-1BB has the amino acid sequence set forth in SEQ ID NO:12, and is encoded by the polynucleotide sequence set forth in SEQ ID NO:155. In some embodiments, the intracellular domain comprises an intracellular costimulatory signaling domain of 4-1BB or functional variant or portion thereof, such as a 42-amino acid cytoplasmic domain of a human 4-1BB (Accession No. Q07011.1) or functional variant or portion thereof, such as the sequence of amino acids set forth in SEQ ID NO: 12 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 12. In some embodiments, a costimulatory molecule can be derived from CD28 and can comprise the amino acid sequence set forth in SEQ ID NO: 10, encoded by the nucleotide sequence set forth in SEQ ID NO: 183. In some embodiments, the intracellular signaling domain comprises an intracellular costimulatory signaling domain of human CD28 or functional variant or portion thereof, such as a 41 amino acid domain thereof and/or such a domain with an LL to GG substitution at positions 186-187 of a native CD28 protein. In some embodiments, the intracellular signaling domain can comprise the sequence of amino acids set forth in SEQ ID NO: 10 or 11 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 10 or 11. In some aspects, the same CAR includes both the stimulatory or activating components (e.g., cytoplasmic signaling sequence) and costimulatory components.

In some aspects, the transmembrane domain contains a transmembrane portion of CD28. The extracellular domain and transmembrane can be linked directly or indirectly. In some embodiments, the extracellular domain and transmembrane are linked by a spacer, such as any described herein. In some embodiments, the chimeric antigen receptor contains an intracellular domain of a T cell costimulatory molecule, such as between the transmembrane domain and intracellular signaling domain. In some aspects, the T cell costimulatory molecule is CD28 or 4-1BB.

In some embodiments, the stimulatory or activating components are included within one CAR, whereas the costimulatory component is provided by another CAR recognizing another antigen. In some embodiments, the CARs include activating or stimulatory CARs, and costimulatory CARs, both expressed on the same cell (see WO 2014/055668). In some aspects, the ROR1-targeting CAR is the stimulatory or activating CAR; in other aspects, it is the costimulatory CAR. In some embodiments, the cells further include inhibitory CARs (iCARs, see Fedorov et al., Sci. Transl. Medicine, 5(215) (December, 2013), such as a CAR recognizing an antigen other than ROR1, whereby a stimulatory or an activating signal delivered through the ROR1-targeting CAR is diminished or inhibited by binding of the inhibitory CAR to its ligand, e.g., to reduce off-target effects.

In some embodiments, the two receptors induce, respectively, an activating and an inhibitory signal to the cell, such that ligation of one of the receptor to its antigen activates the cell or induces a response, but ligation of the second inhibitory receptor to its antigen induces a signal that suppresses or dampens that response. Examples are combinations of activating CARs and inhibitory CARs (iCARs).

Such a strategy may be used, for example, to reduce the likelihood of off-target effects in the context in which the activating CAR binds an antigen expressed in a disease or condition but which is also expressed on normal cells, and the inhibitory receptor binds to a separate antigen which is expressed on the normal cells but not cells of the disease or condition.

In some aspects, the chimeric receptor is or includes an inhibitory CAR (e.g. iCAR) and includes intracellular components that dampen or suppress an immune response, such as an ITAM- and/or co stimulatory-promoted response in the cell. Exemplary of such intracellular signaling components are those found on immune checkpoint molecules, including PD-1, CTLA4, LAG3, BTLA, OX2R, TIM-3, TIGIT, LAIR-1, PGE2 receptors, EP2/4 Adenosine receptors including A2AR. In some aspects, the engineered cell includes an inhibitory CAR including a signaling domain of or derived from such an inhibitory molecule, such that it serves to dampen the response of the cell, for example, that induced by an activating and/or costimulatory CAR.

In certain embodiments, the intracellular signaling region comprises a CD28 transmembrane and signaling domain linked to a CD3 (e.g., CD3-zeta) intracellular domain. In some embodiments, the intracellular signaling domain comprises a chimeric CD28 and 4-1BB (CD137; TNFRSF9) co-stimulatory domains, linked to a CD3 zeta intracellular domain.

In some embodiments, the CAR encompasses one or more, e.g., two or more, costimulatory domains and a stimulatory or an activation domain, e.g., primary activation domain, in the cytoplasmic portion. Exemplary CARs include intracellular components of CD3-zeta, CD28, and 4-1BB.

In some embodiments, provided embodiments of anti-ROR1 CAR contains an extracellular antigen-binding domain containing any of the anti-ROR1 antibody or antigen-binding fragments described herein, such as in Section I.A.1 and/or Table 2; a spacer comprising an IgG4/2 chimeric hinge or a modified IgG4 hinge, such as one that is about 12 amino acids in length, or a spacer set forth in SEQ ID NO:1, such as encoded by the nucleotide sequence set forth in SEQ ID NOS: 2 or 30; a transmembrane domain, such as a transmembrane domain from a human CD28; and an intracellular signaling region comprising a cytoplasmic signaling domain of a CD3-zeta (CD3ζ) chain and a costimulatory signaling region. In some embodiments, provided embodiments of anti-ROR1 CAR contains an extracellular antigen-binding domain containing any of the anti-ROR1 antibody or antigen-binding fragments described herein, such as in Section I.A.1 and/or Table 2; a spacer comprising an IgG4/2 chimeric hinge or a modified IgG4 hinge, such as one that is about 13 amino acids in length, or a spacer set forth in SEQ ID NO:135, such as encoded by the nucleotide sequence set forth in SEQ ID NOS: 136 or 192; a transmembrane domain, such as a transmembrane domain from a human CD28; and an intracellular signaling region comprising a cytoplasmic signaling domain of a CD3-zeta (CD3) chain and a costimulatory signaling region.

In some embodiments, provided embodiments of anti-ROR1 CAR contains an extracellular antigen-binding domain containing any of the anti-ROR1 antibody or antigen-binding fragments described herein, such as in Section I.A.1 and/or Table 2; a spacer comprising a modified IgG4 hinge-C_{H}3, such as one that is about 119 amino acids in length, or a spacer set forth in SEQ ID NO:3, such as encoded by the nucleotide sequence set forth in SEQ ID NO:4 or 137; a transmembrane domain, such as a transmembrane domain from a human CD28; and an intracellular signaling region comprising a cytoplasmic signaling domain of a CD3-zeta (CD3) chain and a costimulatory signaling region. In some embodiments, provided embodiments of anti-ROR1 CAR contains an extracellular antigen-binding domain containing any of the anti-ROR1 antibody or antigen-binding fragments described herein, such as in Section I.A.1 and/or Table 2; a spacer comprising a modified IgG4 hinge-C_{H}3, such as one that is about 120 amino acids in length, or a spacer set forth in SEQ ID NO:138, such as encoded by the nucleotide sequence set forth in SEQ ID NO:193 or 139; a transmembrane domain, such as a transmembrane domain from a human CD28; and an intracellular signaling region comprising a cytoplasmic signaling domain of a CD3-zeta (CD3) chain and a costimulatory signaling region.

In some of any such embodiments, the transmembrane domain is or comprises the sequence set forth in SEQ ID NO:8. In some of any such embodiments, the transmembrane domain is or comprises the sequence set forth in SEQ ID NO:149. In some of any such embodiments, the costimulatory signaling region is an intracellular signaling domain of human CD28, human 4-1BB or human ICOS or a signaling portion thereof. In particular embodiments, the intracellular signaling domain is an intracellular signaling domain of human 4-1BB. In some of any such embodiments, the intracellular signaling domain is or comprises the sequence set forth in SEQ ID NO:12. In some of any such embodiments, the cytoplasmic signaling domain is a human CD3-zeta cytoplasmic signaling domain, such as set forth in SEQ ID NO:13. In some of any such embodiments, the intracellular signaling region comprises the sequences set forth in SEQ ID NO:13 and SEQ ID NO:12. In some of any such embodiments, those described in Section I.D herein, in Table 3 and/or in Table E1. Also provided are CARs encoded by the polynucleotides described in Section I.E herein, in Table 3 and/or in Table E1. Also provided are polynucleotides that contain any of the nucleotide sequences described herein, e.g., encoding all of a portion of the provided binding molecules. In certain cases, the transmembrane domain has a methionine residue at the N-terminus. In certain cases, the spacer has a methionine residue at the C-terminus.

In some of any of the provided embodiments, the anti-ROR1 chimeric antigen receptor is or comprises the sequence set forth in SEQ ID NO: 184, 185, 186, 187, 188 or 189 or a sequence that exhibits at least 85%, 86%, 87%, 88%, 89%, 90%, 91%, 92%, 93%, 94%, 95%, 96%, 97%, 98%, or 99% sequence identity to the sequence set forth in SEQ ID NO: 184, 185, 186, 187, 188 or 189. In some of any embodiments, the anti-ROR1 CARs has the amino acid sequence set forth in SEQ ID NO:184, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO:184. In some of any embodiments, the anti-ROR1 CARs has the amino acid sequence set forth in SEQ ID NO:185, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO:185. In some of any embodiments, the anti-ROR1 CARs has the amino acid sequence set forth in SEQ ID NO:186, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO:186. In some of any embodiments, the anti-ROR1 CARs has the amino acid sequence set forth in SEQ ID NO:187, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO:187. In some of any embodiments, the anti-ROR1 CARs has the amino acid sequence set forth in SEQ ID NO:188, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO:188. In some of any embodiments, the anti-ROR1 CARs has the amino acid sequence set forth in SEQ ID NO:189, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO:189.

In some of any of the provided embodiments, the anti-ROR1 chimeric antigen receptor is encoded by the sequence set forth in SEQ ID NO: 156, 157, 158, 159, 160 or 161 or a sequence that exhibits at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 156, 157, 158, 159, 160 or 161. In some of any embodiments, the anti-ROR1 CARs is encoded by the nucleotide sequence set forth in SEQ ID NO:156, or a nucleotide sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the nucleotide sequence set forth in SEQ ID NO:156. In some of any embodiments, the anti-ROR1 CARs is encoded by the nucleotide sequence set forth in SEQ ID NO:157, or a nucleotide sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the nucleotide sequence set forth in SEQ ID NO:157. In some of any embodiments, the anti-ROR1 CARs is encoded by the nucleotide sequence set forth in SEQ ID NO:158, or a nucleotide sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the nucleotide sequence set forth in SEQ ID NO:158. In some of any embodiments, the anti-ROR1 CARs is encoded by the nucleotide sequence set forth in SEQ ID NO:159, or a nucleotide sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the nucleotide sequence set forth in SEQ ID NO:159. In some of any embodiments, the anti-ROR1 CARs is encoded by the nucleotide sequence set forth in SEQ ID NO:160, or a nucleotide sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the nucleotide sequence set forth in SEQ ID NO:160. In some of any embodiments, the anti-ROR1 CARs is encoded by the nucleotide sequence set forth in SEQ ID NO:161, or a nucleotide sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the nucleotide sequence set forth in SEQ ID NO:161.

### 2. Exemplary Features

In some of any of the provided embodiments, the provided recombinant receptors, e.g., CARs, containing an ROR1-binding antibody or fragment thereof, binds, such as specifically binds to ROR1, such as ROR1 on the surface of a cancer cell or a tumor cell. In any of the embodiments, an antibody or antigen binding fragment, in the provided CARs, specifically binds ROR1, such as a human ROR1. Exemplary ROR1-binding recombinant receptors, e.g., CARs, can exhibit any binding affinity, binding specificity and/or other features of antigen recognition, such as species cross-reactivity, as described herein, such as in Section I.A.2. In some embodiments, a CAR containing an extracellular antigen binding domain comprising the antibodies or fragments thereof exhibit similar or substantially the same binding properties and features as the antibody or fragment thereof. In some cases, the CAR containing an extracellular antigen binding domain comprising the antibodies or fragment thereof exhibit different binding properties and features as the antibody or fragment thereof.

In some embodiments, the provided CARs specifically bind to a receptor tyrosine kinase-like orphan receptor 1 (ROR1) protein. In some of any of the embodiments herein, ROR1 refers to human ROR1. The observation that an antibody or other binding molecule, e.g., CAR, binds to ROR1 or specifically binds to ROR1 does not necessarily mean that it binds to ROR1 from every species. For example, in some embodiments, features of binding to ROR1, such as the ability to specifically bind thereto and/or to compete for binding thereto with a reference receptor, e.g., reference CAR, containing a reference antibody, and/or to bind with a particular affinity or compete to a particular degree, in some embodiments, refers to the ability with respect to a human ROR1 protein and the antibody may not have this feature with respect to a ROR1 of another species such as mouse. In some embodiments, the CAR binds to human ROR1 and binds to ROR1 of another species, such as Rhesus macaque or macaque. In some embodiments, the CAR or an antigen-binding fragment thereof binds to human ROR1 and does not bind to ROR1 of another species, such as mouse. In some embodiments, the CAR binds to human ROR1 and binds to ROR1 of another species, such as mouse.

In some embodiments, the CARs bind, such as specifically bind, to human ROR1, such as to an epitope or region of human ROR1, such as the human ROR1 set forth in SEQ ID NO:144 (GenBank No. AAA60275.1; sequence including the signal peptide set forth in SEQ ID NO:215, Uniprot No. Q01973), or an allelic variant or splice variant thereof. In one embodiment, human ROR1 is a transcript variant or isoform that has the sequence of amino acids forth in SEQ ID NO:145 or 146. In some embodiments, human ROR1 protein comprises an amino acid sequence set forth in SEQ ID NO: 144, 145, 146 or 215. In some embodiments, the CARs bind to the extracellular region ROR1, such as to one or more extracellular epitopes present within the extracellular region of human ROR1, e.g., corresponding to residues 1-377 of the human ROR1 sequence set forth in SEQ ID NO:144 (corresponding to residues 30-406 of the human ROR1 sequence set forth in SEQ ID NO:215 that includes the signal peptide).

In some embodiments, the CAR binds a linear epitope of ROR1, such as a human ROR1. In some embodiments, the one or more epitopes comprises a conformational epitope. In some embodiments, the antibodies or antigen-binding fragment thereof bind one or more conformational epitopes of ROR1, such as a human ROR1. In some embodiments, the antibodies or antigen-binding fragment thereof bind one or more epitopes of human ROR1, such as one or more epitopes that include the sequence FRSTIYGSRLRIRNL (set forth in SEQ ID NO:199) or the sequence set forth in any one of SEQ ID NO:200-214 or in Table E2 herein. In some embodiments, the antibodies or antigen-binding fragment thereof bind one or more epitopes of human ROR1, such as one or more epitopes that include the sequence FRSTIYGSRLRIRNL (set forth in SEQ ID NO:199). In some embodiments, the antibodies or antigen-binding fragment thereof bind additional epitopes, such as one or more conformational epitopes, in addition to the sequence FRSTIYGSRLRIRNL (set forth in SEQ ID NO:199). Exemplary of one or more additional epitopes include, but are not limited to, one or more of the sequence set forth in any one of SEQ ID NO:200-214 or in Table E2 herein.

In some embodiments, the antibody binds to non-human ROR1, such as Rhesus macaques (Macaca mulatta) ROR1 (set forth in SEQ ID NO:216, Uniprot No. F6RUP2) or cynomolgus macaques (Macaca fasicularis) ROR1 (set forth in SEQ ID NO:217, Uniprot No. AOA2K5WTX7; or SEQ ID NO:218, Uniprot No. AOA2K5WTX4). In some aspects, the extracellular domain of the non-human ROR1 is at least 99% identical to the human ROR1 sequence.

In some embodiments, the CAR binds to non-human ROR1, such as monkey, rabbit, rat, mouse, or other species of ROR1. In some embodiments, the CAR binds to mouse (Mus musculus) ROR1, such as to an epitope or region of mouse ROR1, such as the mouse ROR1 set forth in SEQ ID NO: 171 (GenBank No. NP_038873; sequence including the signal peptide set forth in SEQ ID NO:219, Uniprot No. Q9Z139). In some embodiments, the CAR binds to human ROR1 and binds to mouse ROR1. In some embodiments, the extent of binding of some of the provided anti-ROR1 antibodies or fragments thereof to a non-human ROR1, such as mouse ROR1, is at least at or about 75%, 80%, 90%, 95%, 100%, 110%, 120%, 130%, 140%, 150% or more of the binding of the CAR to human ROR1. In some embodiments, the antibodies do not bind to mouse ROR1, such as the mouse ROR1 set forth in SEQ ID NO:171.

In some of any of the provided embodiments, the extent of binding of the CAR, e.g., containing a ROR1-binding antibody or antigen-binding fragment thereof does not bind to, is not cross-reactive to, or binds at a lower extent, level or degree or affinity to a non-human ROR1, optionally a mouse ROR1. In some embodiments, the extent of binding of an anti-ROR1 antibody to an unrelated, non-ROR1 protein or to a non-human ROR1 protein, such as a mouse ROR1 protein, or other non-ROR1 protein, is less than at or about 50%, 40%, 30%, 20% or 10% of the binding of the CAR to human ROR1 as measured. In some embodiments, the antibodies or antigen-binding fragments thereof do not bind to mouse ROR1, such as the mouse ROR1 set forth in SEQ ID NO:171 or 219.

In some of any of the provided embodiments, the CAR or antigen-binding fragment thereof does not bind to, is not cross-reactive to, or binds at a lower extent, level or degree or affinity to a receptor tyrosine kinase-like orphan receptor 2 (ROR2) protein, optionally a human ROR2 protein. In some embodiments, the extent of binding of some of the provided CAR, e.g., containing a ROR1-binding antibody or fragment thereof to a non-ROR1 protein, such as a ROR2 protein, is at least at or about 75%, 80%, 90%, 95% or 99% less than the binding of the CAR to human ROR1. In some embodiments, the provided antibody or antigen-binding fragment thereof does not bind to, is not cross-reactive to, or binds at a lower level or degree or affinity to a ROR2 protein, optionally a human ROR2 protein. In some embodiments, among provided CAR, e.g., containing a ROR1-binding antibody or fragment thereof, are CARs in which binding to mouse ROR1 is less than or at or about 30%, 20% or 10%, such as less than at or about 10%, of the binding of the CAR to human ROR1. In some embodiments, among provided CAR, e.g., containing a ROR1-binding antibody or fragment thereof, are CARs in which binding to a ROR2, such as a human ROR2, is less than or at or about 30%, 20% or 10%, such as less than at or about 10%, of the binding of the CAR to human ROR1. In some embodiments, the provided CARs exhibit the same, substantially the same or lower level or degree or affinity of binding to a ROR2 protein compared to a reference ROR1-specific chimeric antigen receptor, optionally under the same or substantially the same conditions or assay. In some aspects, binding affinity and/or specificity to a particular antigen (e.g., human ROR1) can be assessed using any methods for assessing binding, such as any described in Section I.A.2 herein.

In some embodiments, the provided CARs are capable of binding ROR1, such as human ROR1, with at least a certain affinity, as measured by any of a number of known methods. In some embodiments, the affinity is represented by an equilibrium dissociation constant (K_{D}). In some embodiments, the affinity is represented by EC₅₀.

In some embodiments, the binding molecule, e.g., CAR, binds, such as specifically binds, to an antigen, e.g., a ROR1 protein or an epitope therein, with an affinity or K_{A} (i.e., an equilibrium association constant of a particular binding interaction with units of 1/M or M⁻¹; equal to the ratio of the on-rate [kₒₙ or kₐ] to the off-rate [k_{off} or kₐ] for this association reaction, assuming bimolecular interaction), the binding affinity (EC₅₀) and/or the equilibrium dissociation constant (K_{D}; i.e., an equilibrium dissociation constant of a particular binding interaction with units of M; equal to the ratio of the off-rate [k_{off} or kₐ] to the on-rate [kₒₙ or kₐ] for this association reaction, assuming bimolecular interaction), or an off-rate (dissociation rate constant; k_{off} or kₐ) as described herein, e.g., in Section I.A.2 herein. For example, the equilibrium dissociation constant K_{D} can range from 10⁻⁵ M to 10¹³ M, such as 10⁻⁷ M to 10¹¹ M, 10⁻⁷ M to 10⁻¹⁰ M, 10⁻⁷ M to 10⁹ M, 10⁻⁸ M to 10⁻¹⁰ M, or 10⁹ M to 10⁻¹⁰ M.

In certain embodiments, the binding affinity (EC₅₀) and/or the equilibrium dissociation constant, K_{D}, of the binding molecule, e.g., anti-ROR1 CAR, to a ROR1 protein, such as a human ROR1 protein, is at or about or less than at or about 1 M, 500 nM, 100 nM, 50 nM, 40 nM, 30 nM, 25 nM, 20 nM, 19 nM, 18 nM, 17 nM, 16 nM, 15 nM, 14 nM, 13 nM, 12 nM, 11 nM, 10 nM, 9 nM, 8 nM, 7 nM, 6 nM, 5 nM, 4 nM, 3 nM, 2 nM, or 1 nM or less, or a range defined by any of the foregoing. In some embodiments, the EC₅₀ and/or the K_{D} of the binding molecule, e.g., anti-ROR1 CAR, to a ROR1 protein, is between at or about 10 nM and at or about 90 nM, between at or about 20 nM and at or about 80 nM, between at or about 30 nM and at or about 70 nM, between at or about 40 nM and at or about 60 nM, or between at or about 40 nM and at or about 50 nM. In certain embodiments, the EC₅₀ and/or the K_{D} of the binding molecule, e.g., anti-ROR1 CAR, to a ROR1 protein, such as a human ROR1 protein, is at or about 10 nM, 20 nM, 30 nM, 40 nM, 50 nM, 60 nM, 70 nM, 80 nM, 90 nM or 100 nM, or a range defined by any of the foregoing. In certain embodiments, the EC₅₀ and/or the K_{D} of the binding molecule, e.g., anti-ROR1 CAR, to a ROR1 protein, such as a human ROR1 protein, is at or about 10 nM, 20 nM, 30 nM, 40 nM, 50 nM, 60 nM, 70 nM, 80 nM, 90 nM or 100 nM, or a range defined by any of the foregoing. In certain embodiments, the EC₅₀ and/or the K_{D} of the binding molecule, e.g., anti-ROR1 CAR, to a ROR1 protein, such as a human ROR1 protein, is at or about 40 nM, 41 nM, 42 nM, 43 nM, 44 nM, or 45 nM, or a range defined by any of the foregoing.

In some embodiments, the provided binding molecule, e.g., anti-ROR1 CAR, has a fast off-rate (dissociation rate constant; k_{off} or k_{d}; units of 1/s or s⁻¹). In some embodiments, the off-rate (k_{off} or k_{d}) of the provided binding molecules is between at or about 1×10⁻⁵ s⁻¹ and at or about 1×10⁻² s⁻¹, such as at or about 5×10⁻⁵ s⁻¹ and at or about 9×10⁻³ s⁻¹, at or about 1×10⁻⁴ s⁻¹ and at or about 8×10⁻³ s⁻¹, at or about 5×10⁻⁴ s⁻¹ and at or about 7×10⁻³ s⁻¹, at or about 1×10⁻³ s⁻¹ and at or about 6×10⁻³ s⁻¹, and at or about 4×10⁻³ s⁻¹ and at or about 6×10⁻³ s⁻¹. In some embodiments, the off-rate (k_{off} or k_{d}) of the provided binding molecules is at least at or about 1×10⁻⁵ s⁻¹, 5×10⁻⁵ s⁻¹, 1×10⁻⁴ s⁻¹, 5×10⁻⁴ s⁻¹, 1×10⁻³ s⁻¹, 5×10⁻³ s⁻¹, or 1×10⁻² s⁻¹. In some embodiments, the off-rate (k_{off} or k_{d}) of the provided binding molecules is at least at or about 6×10⁻⁴ s⁻¹, 7×10⁻⁴ s⁻¹, 8×10⁴s⁻¹, 9×10⁻⁴ s⁻¹, 1×10⁻³ s⁻¹, 2×10⁻³ s⁻¹, 3×10⁻³ s⁻¹, 4×10⁻³ s⁻¹, 5×10³ s⁻¹, 6×10³ s⁻¹, 7×10³ s⁻¹, 8×10³ s⁻¹, 9×10³ s⁻¹ or 1×10²s⁻¹. In some embodiments, the off-rate (k_{off} or kₐ) of the provided binding molecules is at least at or about 4×10⁻³ s⁻¹, 5×10⁻³ s 5 or 6×10⁻³ s⁻¹, or a range defined by any of the foregoing. In some embodiments, the provided binding molecule, e.g., anti-ROR1 CAR, has an off-rate that is at least at or about 2-fold, 3-fold, 4-fold, 5-fold, 6-fold, 7-fold, 8-fold, 9-fold or 10-fold faster than the off-rate of a reference anti-ROR1 CAR, for example, anti-ROR1 CAR R12.

In some embodiments, the binding affinity of a binding molecule, such as an anti-ROR1 CAR, for different antigens, e.g., ROR1 proteins from different species can be compared to determine the species cross-reactivity. For example, species cross-reactivity can be classified as high cross reactivity or low cross reactivity. In some embodiments, the equilibrium dissociation constant, K_{D}, for different antigens, e.g., ROR1 proteins from different species such as human, cynomolgus monkey or mouse, can be compared to determine species cross-reactivity. In some embodiments, the species cross-reactivity of an anti-ROR1 CAR can be high, e.g., the anti-ROR1 CAR binds to human ROR1 and a species variant ROR1 to a similar degree, e.g., the ratio of K_{D} for human ROR1 and K_{D} for the species variant ROR1 is or is about 1. In some embodiments, the species cross-reactivity of an anti-ROR1 CAR can be low, e.g., the anti-ROR1 CAR has a high affinity for human ROR1 but a low affinity for a species variant ROR1, or vice versa. For example, the ratio of K_{D} for the species variant ROR1 and K_{D} for the human ROR1 is more than 10, 15, 20, 25, 30, 40, 50, 60, 70, 80, 90, 100, 200, 500, 1000, 2000 or more, and the anti-ROR1 CAR has low species cross-reactivity. The degree of species cross-reactivity can be compared with the species cross-reactivity of a known CAR, such as a reference CAR.

In some embodiments, properties or features of the provided CARs are described in relation to properties observed for another CAR, e.g., a reference CAR. In some embodiments, the reference CAR contains, as an extracellular antigen-binding domain, a reference antibody described herein or fragment thereof, such as, the chimeric rabbit/human IgG1 antibody designated R12 (see, e.g., Yang et al. (2011) PloS ONE, 6:e21018; U.S. Patent Application No. US 2013/0251642); mouse anti-human ROR1 antibody designated 2A2 (see, e.g., Baskar et al. (2012) MAbs, 4:349-361; published U.S. Patent Appl. No. US2012/20058051); humanized anti-ROR1 antibodies described in International PCT Appl. No. WO2014/031174; humanized variant of an antibody designated 99961.

In some embodiments, the CAR has an affinity that is about the same as or lower than that of the corresponding form of the reference CAR, e.g., EC₅₀ or K_{D} that is no more than at or about 1.5-fold or no more than at or about 2-fold greater, no more than at or about 3-fold greater, and/or no more than at or about 10-fold greater, than the EC₅₀ or K_{D} of the corresponding form of the reference CAR. In some embodiments, the CAR has an affinity that is about the same as or lower than that of the corresponding form of the reference CAR, e.g., EC₅₀ or K_{D} that is at least at or about 1.5-fold greater, at least at or about 2-fold greater, at least at or about 3-fold greater, at least at or about 5-fold greater, at least at or about 10-fold greater, at least at or about 20-fold greater, at least at or about 25-fold greater, at least at or about 30-fold greater, at least at or about 40-fold greater, at least at or about 50-fold greater, or at least at or about 100-fold greater, than the EC₅₀ or K_{D} of the corresponding form of the reference CAR. In some embodiments, the CAR has an affinity that is about the same as or lower than that of the corresponding form of the reference CAR, an affinity that is at or about 1.5-fold, 2-fold, 3-fold, 4-fold, 5-fold, 6-fold, 7-fold, 8-fold, 9-fold, 10-fold, 20-fold, 25-fold, 30-fold, 40-fold, 50-fold or 100-fold lower than the affinity of the reference CAR.

In some embodiments, the CAR has an affinity that is greater than that of the corresponding form of the reference CAR, e.g., EC₅₀ or K_{D} that is lower than or lower than at or about 5-fold, 10-fold, 20-fold, 30-fold, 40-fold, 50-fold, 60-fold, 70-fold, 80-fold, 90-fold, 100-fold, 150-fold, 200-fold, 250-fold or lower than the EC₅₀ or K_{D} of the corresponding form of the reference CAR.

In some embodiments, the CAR specifically binds to an epitope that overlaps with the epitope of ROR1 bound by a reference CAR. In some aspects, among such CARs are CARs that bind to the same or a similar epitope as the reference CAR. In some embodiments, the CARs bind to the same or a similar epitope or an epitope within the same region or containing residues within the same region of ROR1 as a reference CAR, such as anti-ROR1 CAR R12 or scFv fragment thereof (set forth in SEQ ID NO: 142; see e.g. Yang et al. (2011) PloS ONE, 6:e21018). In some embodiments, the CAR inhibits binding to and/or competes for binding to ROR1, such as human ROR1, with the reference CAR.

Among the provided CARs are CARs that exhibit antigen-dependent activity or signaling, i.e. signaling activity that is measurably absent or at background levels in the absence of antigen, e.g. ROR1, and/or in the presence of non-specific antigen. Thus, in some aspects, provided CARs do not exhibit, or exhibit no more than background or a tolerable or low level of, tonic signaling or antigen-independent activity or signaling in the absence of antigen, e.g. ROR1, being present. In some embodiments, the provided anti-ROR1 CAR-expressing cells exhibit biological activity or function, including cytotoxic activity, cytokine production, and ability to proliferate. In some embodiments, the provided CARs receptor exhibits the same, substantially the same or higher antigen-specific signaling and/or antigen dependent activity or signaling compared to a reference ROR1-specific chimeric antigen receptor, optionally under the same or substantially the same conditions or assay.

In some embodiments, the provided CARs exhibit absent, reduced or lower levels of activity or signaling in the absence of antigen, e.g., ROR1. In some embodiments, the provided CARs exhibit reduced, lower or is nearly absent of or completely absent of tonic signaling or antigen-independent signaling or activity, e.g., signaling or activity in the absence of antigen, such that the signaling or activity is less than or at or about 30%, 20% or 10%, such as less than at or about 10%, of the signaling or activity of the CAR in the presence of human ROR1. In some embodiments, the provided CARs exhibit the same, substantially the same or lower tonic signaling and/or antigen independent activity or signaling compared to a reference ROR1-specific chimeric antigen receptor (CAR), optionally under the same or substantially the same conditions or assay. In some aspects, among a plurality or population of cells that are engineered to express the CARs, less than at or about 10%, at or about 9%, at or about 8%, at or about 7%, at or about 5%, at or about 4%, at or about 3%, at or about 2% or at or about 1% of the cells in the plurality comprise a chimeric antigen receptor that exhibits tonic signaling and/or antigen independent activity or signaling. In some embodiments, reference ROR1-specific CARs include those that have an antigen-binding domain that comprises any reference anti-ROR1 antibodies described herein, such as those described in Section I.A.2 herein, for example, R12, A2A or 99961, or an antigen-binding fragment thereof.

In some embodiments, the provided CARs exhibit absent, reduced or lower levels of activity or signaling when exposed to a non-specific antigen, such as a different but related protein, such as ROR2. In some embodiments, the provided CARs exhibit the same, substantially the same or lower level or degree or affinity of binding to a ROR2 protein compared to a reference ROR1-specific chimeric antigen receptor, optionally under the same or substantially the same conditions or assay. In some embodiments, the provided CARs exhibit reduced signaling or activity in the presence of a ROR2, such as a human ROR2, such that the signaling or activity is less than or at or about 30%, 20% or 10%, such as less than at or about 10%, of the signaling or activity of the CAR in the presence of human ROR1.

In some embodiments, engineered cells expressing the provided anti-ROR1 CARs exhibit improved biological activity or functional activity, such as anti-tumor activity, tumor growth inhibition, tumor volume reduction, persistence, expansion, or prolonged survival of the subject, when administered to a subject for adoptive cell therapy. In some embodiments, engineered cells expressing the provided CARs exhibit improved biological activity or functional activity, such as anti-tumor activity, tumor growth inhibition, tumor volume reduction, persistence, expansion, or prolonged survival of the subject, compared to engineered cells expressing a reference ROR1-specific chimeric antigen receptor, optionally under the same or substantially the same conditions or assay. In some embodiments, biological activity or functional activity of a chimeric receptor, such as cytotoxic activity, can be measured using any of a number of known methods. The activity can be assessed or determined either in vitro or in vivo. In some embodiments, activity can be assessed once the cells are administered to the subject (e.g., human). Parameters to assess include specific binding of an engineered or natural T cell or other immune cell to antigen, e.g., in vivo, e.g., by imaging, or ex vivo, e.g., by ELISA or flow cytometry. In certain embodiments, the ability of the engineered cells to destroy target cells can be measured using any suitable known methods, such as cytotoxicity assays described in, for example, Kochenderfer et al., J. Immunotherapy, 32(7): 689-702 (2009), and Herman et al. J. Immunological Methods, 285(1): 25-40 (2004). In certain embodiments, the biological activity of the cells also can be measured by assaying expression and/or secretion of certain cytokines, such as interleukin-2 (IL-2), interferon-gamma (IFNγ), interleukin-4 (IL-4), TNF-alpha (TNFα), interleukin-6 (IL-6), interleukin-10 (IL-10), interleukin-12 (IL-12), granulocyte-macrophage colony-stimulating factor (GM-CSF), CD107a, and/or TGF-beta (TGFβ). Assays to measure cytokines are well known, and include but are not limited to, ELISA, intracellular cytokine staining, cytometric bead array, RT-PCR, ELISPOT, flow cytometry and bio-assays in which cells responsive to the relevant cytokine are tested for responsiveness (e.g. proliferation) in the presence of a test sample. In some aspects the biological activity can be measured using an animal model of the disease or condition, such as a tumor xenograft model, and assessing the reduction in tumor burden or load and/or survival. In some aspects the biological activity is measured by assessing clinical outcome, such as reduction in tumor burden or load.

In some embodiments, administration of engineered cells expressing the provided anti-ROR1 CARs exhibit substantially improved tumor growth inhibition, in vivo persistence and/or prolonged survival of the subject with a tumor, compared to administration of engineered cells expressing reference CAR, such as anti-ROR1 R12. In some aspects, such improvement is observed in subjects having various types of ROR1-expressing cancers, including, but not limited to, lung cancer, breast cancer, chronic lymphocytic leukemia (CLL), ovarian cancer or mantle cell lymphoma (MCL).

In some embodiments, engineered cells expressing the provided anti-ROR1 CARs exhibit improved persistence and expansion when administered to a subject for adoptive cell therapy. In some embodiments, engineered cells expressing the provided CARs exhibit the same, substantially the same or higher persistence and expansion compared to engineered cells expressing a reference ROR1-specific chimeric antigen receptor, optionally under the same or substantially the same conditions or assay. In some aspects, the proliferation, expansion and/or persistence of an engineered cell expressing any of the provided receptors, e.g., CARs, can be assessed by determining the exposure, number, concentration, persistence and proliferation of the engineered cells, e.g., cells administered for adoptive cell therapy. In some embodiments, the exposure, number or level of engineered T cells, e.g., T cells administered for the T cell based therapy, or subset thereof, such as CD3⁺ cells, CD4⁺ cells, CD8⁺ cells, CD3⁺ CAR⁺ cells, CD4⁺ CAR⁺ cells or CD8⁺ CAR⁺ cells can be assessed, e.g., from a subject, such as a human subject or an animal subject, that had been administered engineered cells. In some aspects, the exposure, number, concentration, persistence and proliferation relate to pharmacokinetic parameters. In some cases, pharmacokinetics can be assessed by measuring such parameters as the maximum (peak) plasma concentration (Cₘₐₓ), the peak time (i.e. when maximum plasma concentration (Cₘₐₓ) occurs; Tₘₐₓ), the minimum plasma concentration (i.e. the minimum plasma concentration between doses of a therapeutic agent, e.g., CAR⁺ T cells; Cₘᵢₙ), the elimination half-life (T_{1/2}) and area under the curve (i.e. the area under the curve generated by plotting time versus plasma concentration of the therapeutic agent CAR⁺ T cells; AUC), following administration. The concentration of a particular therapeutic agent, e.g., CAR+ T cells, in the plasma following administration can be measured using any known methods suitable for assessing concentrations of the therapeutic agents, e.g., CAR⁺ T cells, in samples of blood, or any methods described herein. For example, nucleic acid-based methods, such as quantitative PCR (qPCR) or flow cytometry-based methods, or other assays, such as an immunoassay, ELISA, or chromatography/mass spectrometry-based assays can be used.

In some aspects, a reporter cell line can be employed to monitor antigen-independent activity and/or tonic signaling through anti-ROR1 CAR-expressing cells. In some embodiments, a T cell line, such as a Jurkat cell line, contains a reporter molecule, such as a fluorescent protein or other detectable molecule, such as a red fluorescent protein, expressed under the control of the endogenous Nur77 transcriptional regulatory elements. In some embodiments, the Nur77 reporter expression is cell intrinsic and dependent upon signaling through a recombinant reporter containing a primary activation signal in a T cell, a signaling domain of a T cell receptor (TCR) component, and/or a signaling domain comprising an immunoreceptor tyrosine-based activation motif (ITAM), such as a CD3ζ chain. Nur77 expression is generally not affected by other signaling pathways such as cytokine signaling or toll-like receptor (TLR) signaling, which may act in a cell extrinsic manner and may not depend on signaling through the recombinant receptor. Thus, only cells that express the exogenous recombinant receptor, e.g., anti-ROR1 CAR, containing the appropriate signaling regions is capable of expressing Nur77 upon stimulation (e.g., binding of the specific antigen). In some cases, Nur77 expression also can show a dose-dependent response to the amount of stimulation (e.g., antigen).

In some cases, to assess the specificity, cross-reactivity and/or antigen dependency of a particular activity, signaling or function of the receptor, e.g., CAR, any of the described assays for assessing activity, signaling or biological function of the receptors can be assessed in the presence and absence of the specific target antigen, e.g., human ROR1, or in the presence of the specific target antigen, e.g., human ROR1, and in the presence of a different, non-specific antigen, e.g., human ROR2 or a non-human ROR1, such as a mouse ROR1.

In some of any embodiments, the provided anti-ROR1 CAR exhibits the same, substantially the same or lower level or degree of signaling or activity in the presence of a ROR2 protein, e.g., a human ROR2, compared to the level or degree of signaling or activity in the presence of a ROR1 protein, e.g., a human ROR1, e.g., under the same or substantially the same conditions or assay. For example, in some aspects, the provided anti-ROR1 CAR exhibits a level or degree of signaling or activity in the presence of a human ROR2 that is at least at or about 75%, 80%, 90%, 95% or 99% less than the level or degree of signaling or activity in the presence of a human ROR1, e.g., under the same or substantially the same conditions or assay.

In some of any embodiments, the provided anti-ROR1 CAR exhibits the same, substantially the same or lower level or degree of signaling or activity in the presence of a ROR2 protein, e.g., a human ROR2, compared to a reference ROR1-specific CAR, e.g., under the same or substantially the same conditions or assay. In some of any embodiments, the provided anti-ROR1 CAR exhibits the same, substantially the same or lower level or degree of signaling or activity in the presence of a ROR2 protein, e.g., a human ROR2, compared to a reference ROR1-specific CAR, e.g., under the same or substantially the same conditions or assay. In some of any embodiments, the provided anti-ROR1 CAR exhibits the same, substantially the same or higher antigen-specific signaling and/or antigen dependent activity or signaling compared to a reference ROR1-specific CAR, e.g., under the same or substantially the same conditions or assay.

In some of any embodiments, the provided anti-ROR1 CAR exhibits the same, substantially the same or lower tonic signaling and/or antigen independent activity or signaling compared to a reference ROR1-specific CAR, e.g., under the same or substantially the same conditions or assay. For example, in some embodiments, the provided anti-ROR1 CAR exhibits a level or degree of tonic signaling and/or antigen independent activity or signaling that is at least at or about 75%, 80%, 90%, 95% or 99% less than the level or degree of tonic signaling and/or antigen independent activity of a reference ROR1-specific CAR, e.g., under the same or substantially the same conditions or assay.

In some embodiments, the provided anti-ROR1 CARs exhibit improved expression on the surface of cells, such as compared to an alternative CAR that has an identical amino acid sequence but that is encoded by non-splice site eliminated and/or a codon-optimized nucleotide sequence. In some embodiments, the expression of the recombinant receptor on the surface of the cell can be assessed. Approaches for determining expression of the recombinant receptor on the surface of the cell may include use of chimeric antigen receptor (CAR)-specific antibodies (e.g., Brentjens et al., Sci. Transl. Med. 2013 March; 5(177): 177ra38), Protein L (Zheng et al., J. Transl. Med. 2012 February; 10:29), epitope tags, and monoclonal antibodies that specifically bind to a CAR polypeptide (see WO2014190273). In some embodiments, the expression of the recombinant receptor on the surface of the cell, e.g., primary T cell, can be assessed, for example, by flow cytometry, using binding molecules that can bind to the recombinant receptor or a portion thereof that can be detected. In some embodiments, the binding molecules used for detecting expression of the recombinant receptor an anti-idiotypic antibody, e.g., an anti-idiotypic agonist antibody specific for a binding domain, e.g., scFv, or a portion thereof. In some embodiments, the binding molecule is or comprises an isolated or purified antigen, e.g., recombinantly expressed antigen.

### E. Polynucleotides Encoding Binding Molecules

Also provided are polynucleotides encoding the binding molecules, such as anti-ROR1 antibodies, antigen-binding fragments thereof, recombinant receptors (e.g., chimeric antigen receptors) and/or portions, e.g., chains or fragments, thereof. Among the provided polynucleotides are those encoding the anti-ROR1 antibodies (e.g., antigen-binding fragment) or chimeric antigen receptors described herein. The polynucleotides may include those encompassing natural and/or non-naturally occurring nucleotides and bases, e.g., including those with backbone modifications. The terms “nucleic acid molecule”, “nucleic acid”, “sequence of nucleotides”, and “polynucleotide” may be used interchangeably, and refer to a polymer of nucleotides. Such polymers of nucleotides may contain natural and/or non-natural nucleotides, and include, but are not limited to, DNA, RNA, and PNA. “Nucleic acid sequence” refers to the linear sequence of nucleotides that comprise the nucleic acid molecule or polynucleotide.

Provided are polynucleotides that contain a nucleic acid encoding any of the anti-ROR1 antibody or antigen-binding domain thereof described herein, or any portion, fragment, chain or domain thereof. In some embodiments, the antibody or antigen-binding fragment thereof contain multiple domains or chains (e.g., heavy chain and a light chain), and all of the antibody or antigen-binding fragment thereof is encoded in one polynucleotide. Also provided are polynucleotides that contain a nucleic acid encoding any of the single chain cell surface proteins described herein. Also provided are polynucleotides that contain a nucleic acid encoding any of the conjugate described herein. Also provided are polynucleotides that contain a nucleic acid encoding any of the anti-ROR1 chimeric antigen receptors described herein. In some embodiments, the binding molecule, such as the antibody or antigen-binding fragment thereof or recombinant receptors, contain multiple domains or chains (e.g., a heavy chain and a light chain), and all of the binding molecule is encoded in more than one polynucleotide, such as two or more polynucleotides. In some embodiments, the polynucleotides are comprised in a vector.

In some aspects, provided are polynucleotides that contain nucleic acid sequences encoding any of the binding molecules provided herein, for example, in Section I.A and I.D. In some embodiments, provided are polynucleotides that contain nucleic acid sequences encoding a portion, fragment, chain or domain of any of the binding molecules provided herein, for example, in Section I.A and I.D.

In some cases, the polynucleotide encoding the ROR1-binding molecules, such as an antibody or antigen-binding fragment thereof or a recombinant receptor (e.g., CAR) contains a signal sequence that encodes a signal peptide, in some cases encoded upstream of the nucleic acid sequences encoding the ROR1-binding molecules, such as an antibody or antigen-binding fragment thereof or a recombinant receptor (e.g., CAR), or joined at the 5′ terminus of the nucleic acid sequences encoding the antigen-binding domain. In some cases, the polynucleotide containing nucleic acid sequences encoding the ROR1-binding molecules, such as an antibody or antigen-binding fragment thereof or a recombinant receptor (e.g., CAR), contains a signal sequence that encodes a signal peptide. In some aspects, the signal sequence may encode a signal peptide derived from a native polypeptide. In other aspects, the signal sequence may encode a heterologous or non-native signal peptide. In some aspects, non-limiting exemplary signal peptide include a signal peptide of the IgG kappa chain set forth in SEQ ID NO: 43 or encoded by the nucleotide sequence set forth in SEQ ID NO:44. In some aspects, a non-limiting exemplary signal peptide includes a signal peptide of a GMCSFR alpha chain set forth in SEQ ID NO:45 and encoded by the nucleotide sequence set forth in SEQ ID NO:46. In some aspects, a non-limiting exemplary signal peptide includes a signal peptide of a CD8 alpha signal peptide set forth in SEQ ID NO:47. In some aspects, a non-limiting exemplary signal peptide includes a signal peptide of a CD33 signal peptide set forth in SEQ ID NO:42 and encoded by the nucleotide sequence set forth in SEQ ID NO:190. In some cases, the polynucleotide encoding the ROR1-binding molecules, such as an antibody or antigen-binding fragment thereof or a recombinant receptor (e.g., CAR) can contain nucleic acid sequence encoding additional molecules, such as a surrogate marker or other markers, or can contain additional components, such as promoters, regulatory elements and/or multicistronic elements. In some embodiments, the nucleic acid sequence encoding the ROR1-binding molecules, such as an antibody or antigen-binding fragment thereof or a recombinant receptor (e.g., CAR) can be operably linked to any of the additional components.

In some embodiments, provided are polynucleotides contain nucleic acid sequences encoding a variable heavy chain domain (V_{H}) of an antibody or an antigen-binding fragment thereof, or a recombinant receptor containing an antibody or an antigen-binding fragment thereof. In some embodiments, provided are polynucleotides contain nucleic acid sequences encoding a variable light chain domain (V_{L}) of an antibody or an antigen-binding fragment thereof, or a recombinant receptor containing an antibody or an antigen-binding fragment thereof. In some embodiments, provided are polynucleotides that contain nucleic acid sequences encoding a variable heavy chain domain (V_{H}) and a variable light chain domain (V_{L}) of an antibody or an antigen-binding fragment thereof, or a recombinant receptor containing an antibody or an antigen-binding fragment thereof.

In some of any embodiments, said polynucleotide contains a nucleic acid encoding the V_{H} comprising the sequence set forth in SEQ ID NO: 110, 119, 101 or 128 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 110, 119, 101 or 128. In some of any embodiments, said polynucleotide contains a nucleic acid encoding the V_{L} comprising the sequence set forth in SEQ ID NO: 113, 122 or 104, or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 113, 122 or 104.

In some of any embodiments, said polynucleotide contains a nucleic acid encoding the V_{H} comprising the sequence set forth in SEQ ID NO: 110, 119, 101 or 128 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 110, 119, 101 or 128, and a nucleic acid encoding the V_{L} comprising the sequence set forth in SEQ ID NO: 113, 122 or 104, or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 113, 122 or 104.

In some of any embodiments, said polynucleotide contains a nucleic acid encoding the V_{H} comprising the sequence set forth in SEQ ID NO: 110 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 110. In some embodiments, said polynucleotide contains a nucleic acid encoding the V_{L} comprising the sequence set forth in SEQ ID NO: 113, or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 113. In some of any embodiments, said polynucleotide contains a nucleic acid encoding the V_{H} comprising the sequence set forth in SEQ ID NO: 119 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 119. In some embodiments, said polynucleotide contains a nucleic acid encoding the V_{L} comprising the sequence set forth in SEQ ID NO: 122, or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 122.

In some of any embodiments, said polynucleotide contains a nucleic acid encoding the V_{H} comprising the sequence set forth in SEQ ID NO: 101 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 101. In some embodiments, said polynucleotide contains a nucleic acid encoding the V_{L} comprising the sequence set forth in SEQ ID NO: 104, or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 104. In some of any embodiments, said polynucleotide contains a nucleic acid encoding the V_{H} comprising the sequence set forth in SEQ ID NO: 128 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 128. In some embodiments, said polynucleotide contains a nucleic acid encoding the V_{L} comprising the sequence set forth in SEQ ID NO: 104, or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 104.

In some of any embodiments, said polynucleotide contains a nucleic acid encoding the V_{H} comprising the sequence set forth in SEQ ID NO: 110 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 110, and a nucleic acid encoding the V_{L} comprising the sequence set forth in SEQ ID NO: 113, or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 113. In some of any embodiments, said polynucleotide contains a nucleic acid encoding the V_{H} comprising the sequence set forth in SEQ ID NO: 119 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 119, and a nucleic acid encoding the V_{L} comprising the sequence set forth in SEQ ID NO: 122, or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 122.

In some of any embodiments, said polynucleotide contains a nucleic acid encoding the V_{H} comprising the sequence set forth in SEQ ID NO: 101 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 101, and a nucleic acid encoding the V_{L} comprising the sequence set forth in SEQ ID NO: 104, or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 104. In some of any embodiments, said polynucleotide contains a nucleic acid encoding the V_{H} comprising the sequence set forth in SEQ ID NO: 128 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 128, and a nucleic acid encoding the V_{L} comprising the sequence set forth in SEQ ID NO: 104, or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 104.

In some of any embodiments, said polynucleotide contains a nucleic acid encoding the scFv comprising the sequence set forth in SEQ ID NO: 116, 125, 107 or 132 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 116, 125, 107 or 132. In some of any embodiments, said polynucleotide contains a nucleic acid encoding the scFv comprising the sequence set forth in SEQ ID NO: 116 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 116. In some of any embodiments, said polynucleotide contains a nucleic acid encoding the scFv comprising the sequence set forth in SEQ ID NO: 125 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 125. In some of any embodiments, said polynucleotide contains a nucleic acid encoding the scFv comprising the sequence set forth in SEQ ID NO: 107 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 107. In some of any embodiments, said polynucleotide contains a nucleic acid encoding the scFv comprising the sequence set forth in SEQ ID NO: 132 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 132.

In some of any embodiments, said polynucleotide contains a nucleic acid encoding the spacer comprising the sequence set forth in SEQ ID NO: 30 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 30. In some of any embodiments, said polynucleotide contains a nucleic acid encoding the spacer comprising the sequence set forth in SEQ ID NO: 192 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 192. In some of any embodiments, said polynucleotide contains a nucleic acid encoding the spacer comprising the sequence set forth in SEQ ID NO: 193 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 193. In some of any embodiments, said polynucleotide contains a nucleic acid encoding the spacer comprising the sequence set forth in SEQ ID NO: 195 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 195.

Also provided are polynucleotides that have been optimized for codon usage and/or to eliminate splice sites, such as cryptic splice sites. In some embodiments, the polynucleotides are modified to optimize codon usage. In some embodiments, the polynucleotides are codon optimized for expression in a human cell such as a human T cell such as a primary human T cell. In some embodiments, the polynucleotides, such as those encoding any of the antibodies, receptors (such as antigen receptors such as chimeric antigen receptors) and/or ROR1-specific binding proteins provided herein, are or have been modified to reduce heterogeneity or contain one or more nucleic acid sequences observed herein (such as by the optimization methods) to result in improved features of the polypeptides, such as the CARs, as compared to those containing distinct, reference, sequences or that have not been optimized. In some embodiments, the polynucleotide is optimized by splice site elimination. Among such features include improvements in RNA heterogeneity, such as that resulting from the presence of one or more splice sites, such as one or more cryptic splice sites, and/or improved expression and/or surface expression of the encoded protein, such as increased levels, uniformity, or consistency of expression among cells or different therapeutic cell compositions engineered to express the polypeptides. In some embodiments, the polynucleotides can be codon optimized for expression in human cells.

Genomic nucleic acid sequences generally, in nature, in a mammalian cell, undergo processing co-transcriptionally or immediately following transcription, wherein a nascent precursor messenger ribonucleic acid (pre-mRNA), transcribed from a genomic deoxyribonucleic acid (DNA) sequence, is in some cases edited by way of splicing, to remove introns, followed by ligation of the exons in eukaryotic cells. Consensus sequences for splice sites are known, but in some aspects, specific nucleotide information defining a splice site may be complex and may not be readily apparent based on available methods. Cryptic splice sites are splice sites that are not predicted based on the standard consensus sequences and are variably activated. Hence, variable splicing of pre-mRNA at cryptic splice sites leads to heterogeneity in the transcribed mRNA products upon expression in eukaryotic cells.

Polynucleotides generated for the expression of transgenes are typically constructed from nucleic acid sequences, such as complementary DNA (cDNA), or portions thereof, that do not contain introns. Thus, splicing of such sequences is not expected to occur. However, the presence of cryptic splice sites within the cDNA sequence can lead to unintended or undesired splicing reactions and heterogeneity in the transcribed mRNA. Such heterogeneity results in translation of unintended protein products, such as truncated protein products with variable amino acid sequences that exhibit modified expression and/or activity.

In some embodiments, eliminating splice sites, such as cryptic splice sites, can improve or optimize expression of a transgene product, such as a polypeptide translated from the transgene, such as an anti-ROR1 CAR polypeptide. Splicing at cryptic splice sites of an encoded transgene, such as an encoded ROR1 CAR molecule, can lead to reduced protein expression, e.g., expression on cell surfaces, and/or reduced function, e.g., reduced intracellular signaling. Provided herein are polynucleotides, encoding anti-ROR1 CAR proteins that have been optimized to reduce or eliminate cryptic splice sites. Also provided herein are polynucleotides encoding anti-ROR1 CAR proteins that have been optimized for codon expression and/or in which one or more sequence, such as one identified by the methods or observations herein regarding splice sites, is present, and/or in which an identified splice site, such as any of the identified splice sites herein, is not present. Among the provided polynucleotides are those exhibiting below a certain degree of RNA heterogeneity or splice forms when expressed under certain conditions and/or introduced into a specified cell type, such as a human T cell, such as a primary human T cell, and cells and compositions and articles of manufacture containing such polypeptides and/or exhibiting such properties. In some embodiments, the RNA heterogeneity of transcribed RNA is reduced by greater than or greater than about 10%, 15%, 20%, 25%, 30%, 40%, 50% or more compared to a polynucleotide that has not been modified to remove cryptic splice sites and/or by codon optimization. In some embodiments, the provided polynucleotides encoding an anti-ROR1 CAR exhibit RNA homogeneity of transcribed RNA that is at least 70%, 75%, 80%, 85%, 90%, or 95% or greater.

RNA heterogeneity can be determined by any of a number of methods provided herein or described or known. In some embodiments, RNA heterogeneity of a transcribed nucleic acid is determined by amplifying the transcribed nucleic acid, such as by reverse transcriptase polymerase chain reaction (RT-PCR) followed by detecting one or more differences, such as differences in size, in the one or more amplified products. In some embodiments, the RNA heterogeneity is determined based on the number of differently sized amplified products, or the proportion of various differently sized amplified products. In some embodiments, RNA, such as total RNA or cytoplasmic polyadenylated RNA, is harvested from cells, expressing the transgene to be optimized, and amplified by reverse transcriptase polymerase chain reaction (RT-PCR) using a primer specific to the 5′ untranslated region (5′ UTR), in some cases corresponding to a portion of the promoter sequence in the expression vector, located upstream of the transgene in the transcribed RNA, and a primer specific to the 3′ untranslated region (3′ UTR), located downstream of the expressed transgene in the transcribed RNA sequence or a primer specific to a sequence within the transgene. In particular embodiments, at least one primer complementary to a sequence in the 5′ untranslated region (UTR) and at least one primer complementary to a sequence in the 3′ untranslated region (UTR) are employed to amplify the transgene. One can resolve RNA, such as messenger RNA, and analyze the heterogeneity thereof by several methods. Non-limiting, exemplary methods include agarose gel electrophoresis, chip-based capillary electrophoresis, analytical centrifugation, field flow fractionation, and chromatography, such as size exclusion chromatography or liquid chromatography.

In some embodiments, a provided polynucleotide encoding an anti-ROR1 CAR provided herein, or a construct provided herein, includes modifications to remove one or more splice donor and/or acceptor site that may contribute to splice events and/or reduced expression and/or increased RNA heterogeneity. In some embodiments, provided polynucleotides are modified in one or more polynucleotides in the spacer region to eliminate or reduce splice events.

In some of any embodiments, said polynucleotide contains a nucleic acid encoding the V_{H} comprising the sequence set forth in SEQ ID NO: 111, 120, 102 or 129, and a nucleic acid encoding the V_{L} comprising the sequence set forth in SEQ ID NO: 114, 123, 105 or 131. In some of any embodiments, said polynucleotide contains a nucleic acid encoding the V_{H} comprising the sequence set forth in SEQ ID NO: 111, and a nucleic acid encoding the V_{L} comprising the sequence set forth in SEQ ID NO: 114. In some of any embodiments, said polynucleotide contains a nucleic acid encoding the V_{H} comprising the sequence set forth in SEQ ID NO: 120, and a nucleic acid encoding the V_{L} comprising the sequence set forth in SEQ ID NO: 123. In some of any embodiments, said polynucleotide contains a nucleic acid encoding the V_{H} comprising the sequence set forth in SEQ ID NO: 102, and a nucleic acid encoding the V_{L} comprising the sequence set forth in SEQ ID NO: 105. In some of any embodiments, said polynucleotide contains a nucleic acid encoding the V_{H} comprising the sequence set forth in SEQ ID NO: 129, and a nucleic acid encoding the V_{L} comprising the sequence set forth in SEQ ID NO: 131.

In some of any embodiments, said polynucleotide contains a nucleic acid encoding the scFv comprising the sequence set forth in SEQ ID NO: 117, 126, 108 or 133. In some of any embodiments, said polynucleotide contains a nucleic acid encoding the scFv comprising the sequence set forth in SEQ ID NO: 117. In some of any embodiments, said polynucleotide contains a nucleic acid encoding the scFv comprising the sequence set forth in SEQ ID NO: 126. In some of any embodiments, said polynucleotide contains a nucleic acid encoding the scFv comprising the sequence set forth in SEQ ID NO: 108. In some of any embodiments, said polynucleotide contains a nucleic acid encoding the scFv comprising the sequence set forth in SEQ ID NO: 133.

In some of any embodiments, said polynucleotide contains a nucleic acid encoding the spacer comprising the sequence set forth in SEQ ID NO:136. In some of any embodiments, said polynucleotide contains a nucleic acid encoding the spacer comprising the sequence set forth in SEQ ID NO:139. In some of any embodiments, said polynucleotide contains a nucleic acid encoding the spacer comprising the sequence set forth in SEQ ID NO:196.

Provided in are exemplary chimeric antigen receptors (CARs) specific for ROR1, and polynucleotides containing nucleic acid sequences encoding all or a portion, fragment, domain or chain of any of the exemplary CARs described herein. In some embodiments, the exemplary CARs contain one of the ROR1-binding antibody fragment, such as those described in Section I.A and/or Table 2. In some embodiments, the exemplary CARs are among those described in each row of Table E1 and/or Table 3. Also provided herein are polynucleotides encoding the CARs. In some embodiments, the CAR can be encoded by more than one different polynucleotides, such as two or more polynucleotides. In some of any such embodiments, two or more polynucleotides can each contain nucleic acids encoding a portion, fragment, domain or chain of the CAR.

Also provided herein are exemplary modified polynucleotides, including polynucleotides that were modified for codon optimization (O) and/or splice site elimination (SSE). Examples of the SEQ ID NOS: for such polynucleotides are set forth in Table 3, wherein exemplary nucleotide (nt) sequences for the components of the exemplary CAR constructs prior to splice site elimination and codon optimization (N/O), nucleic acid (nt) sequences for the components of the CAR constructs following splice site elimination and optimization (O/SSE), and the corresponding amino acid (aa) sequences encoded by the nucleic acid sequences are provided. The components include the anti-ROR1 scFv, spacer, transmembrane (tm) domain, 4-1BB costimulatory signaling region sequence (costim), CD3ζ signaling domain (CD3ζ. The full sequences of the exemplary CARs following splice site elimination and optimization (CAR) are also provided. In some cases, the polynucleotide also includes the CD33 signal sequence (ss), a T2A ribosomal skip element (T2A) and truncated EGF receptor (EGFRt) sequence. Polynucleotide sequences of exemplary CAR are set forth in SEQ ID NOs: 156-161, encoding the amino acid sequences set forth in SEQ ID NOs: 184-189.

In some of any embodiments, said polynucleotide contains the sequence set forth in SEQ ID NO: 156, 157, 158, 159, 160 or 161 or a sequence that exhibits at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 156, 157, 158, 159, 160 or 161. In some of any embodiments, said polynucleotide contains the sequence set forth in SEQ ID NO: 156 or a sequence that exhibits at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 156. In some of any embodiments, said polynucleotide contains the sequence set forth in SEQ ID NO: 156. In some of any embodiments, said polynucleotide contains the sequence set forth in SEQ ID NO: 157 or a sequence that exhibits at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 157. In some of any embodiments, said polynucleotide contains the sequence set forth in SEQ ID NO: 157. In some of any embodiments, said polynucleotide contains the sequence set forth in SEQ ID NO: 158 or a sequence that exhibits at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 158. In some of any embodiments, said polynucleotide contains the sequence set forth in SEQ ID NO: 158. In some of any embodiments, said polynucleotide contains the sequence set forth in SEQ ID NO: 159 or a sequence that exhibits at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 159. In some of any embodiments, said polynucleotide contains the sequence set forth in SEQ ID NO: 159. In some of any embodiments, said polynucleotide contains the sequence set forth in SEQ ID NO: 160 or a sequence that exhibits at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 160. In some of any embodiments, said polynucleotide contains the sequence set forth in SEQ ID NO: 160. In some of any embodiments, said polynucleotide contains the sequence set forth in SEQ ID NO: 161 or a sequence that exhibits at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 161. In some of any embodiments, said polynucleotide contains the sequence set forth in SEQ ID NO: 161.

In some of any embodiments, said polynucleotide contains a nucleic acid encoding the sequence set forth in SEQ ID NO: 184, 185, 186, 187, 188 or 189 or a sequence that encodes a polypeptide sequence that exhibits at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 184, 185, 186, 187, 188 or 189. In some of any embodiments, said polynucleotide contains a nucleic acid encoding the sequence set forth in SEQ ID NO: 184 or a sequence that encodes a polypeptide sequence that exhibits at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 184. In some of any embodiments, said polynucleotide contains a nucleic acid encoding the sequence set forth in SEQ ID NO: 184. In some of any embodiments, said polynucleotide contains a nucleic acid encoding the sequence set forth in SEQ ID NO: 185 or a sequence that encodes a polypeptide sequence that exhibits at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 185. In some of any embodiments, said polynucleotide contains a nucleic acid encoding the sequence set forth in SEQ ID NO: 185. In some of any embodiments, said polynucleotide contains a nucleic acid encoding the sequence set forth in SEQ ID NO: 186 or a sequence that encodes a polypeptide sequence that exhibits at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 186. In some of any embodiments, said polynucleotide contains a nucleic acid encoding the sequence set forth in SEQ ID NO: 186. In some of any embodiments, said polynucleotide contains a nucleic acid encoding the sequence set forth in SEQ ID NO: 187 or a sequence that encodes a polypeptide sequence that exhibits at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 187. In some of any embodiments, said polynucleotide contains a nucleic acid encoding the sequence set forth in SEQ ID NO: 187. In some of any embodiments, said polynucleotide contains a nucleic acid encoding the sequence set forth in SEQ ID NO: 188 or a sequence that encodes a polypeptide sequence that exhibits at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 188. In some of any embodiments, said polynucleotide contains a nucleic acid encoding the sequence set forth in SEQ ID NO: 188. In some of any embodiments, said polynucleotide contains a nucleic acid encoding the sequence set forth in SEQ ID NO: 189 or a sequence that encodes a polypeptide sequence that exhibits at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 189. In some of any embodiments, said polynucleotide contains a nucleic acid encoding the sequence set forth in SEQ ID NO: 189.

TABLE 3
Exemplary ROR1-binding CAR components (SEQ ID NOS:)

| | | Nucleotides (nt) | | | | | | | | | | |
|---|---|---|---|---|---|---|---|---|---|---|---|---|
| | | scFv | | spacer | | TM | | 4-1BB | | CD3ξ | | CAR |
| CAR # | scFv# | N/O | O/SSE | N/O | O/SSE | N/O | O/SSE | N/O | O/SSE | N/O | O/SSE | O/SSE |
| F | ROR1-1 | 116 | 117 | 192 | 136 | 197 | 198 | 154 | 155 | 182 | 150 | 156 |
| A | ROR1-2 | 125 | 126 | 192 | 136 | 197 | 198 | 154 | 155 | 182 | 150 | 157 |
| G | ROR1-1 | 116 | 117 | 193 | 139 | 197 | 198 | 154 | 155 | 182 | 150 | 158 |
| I | ROR1-3 | 107 | 108 | 193 | 139 | 197 | 198 | 154 | 155 | 182 | 150 | 159 |
| R | ROR1-2 | 125 | 126 | 193 | 139 | 197 | 198 | 154 | 155 | 182 | 150 | 160 |
| B1 | ROR1-4 | 132 | 133 | 193 | 139 | 197 | 198 | 154 | 155 | 182 | 150 | 161 |
| | | Amino acids (aa) | | | | | | | | | | |
| CAR # | scFv# | scFv | spacer | TM | 4-1BB | CD3z | CAR | | | | | |
| F | ROR1-1 | 118 | 135 | 8 | 12 | 13 | 184 | | | | | |
| A | ROR1-2 | 127 | 135 | 8 | 12 | 13 | 185 | | | | | |
| G | ROR1-1 | 118 | 138 | 8 | 12 | 13 | 186 | | | | | |
| I | ROR1-3 | 109 | 138 | 8 | 12 | 13 | 187 | | | | | |
| R | ROR1-2 | 127 | 138 | 8 | 12 | 13 | 188 | | | | | |
| B1 | ROR1-4 | 134 | 138 | 8 | 12 | 13 | 189 | | | | | |

### II. ENGINEERED CELLS

Also provided are cells such as engineered cells that contain a recombinant receptor (e.g., a chimeric antigen receptor) such as one that contains an extracellular domain including an anti-ROR1 antibody or fragment as described herein. Also provided are populations of such cells, compositions containing such cells and/or enriched for such cells, such as in which cells expressing the ROR1-binding molecule make up at least 50, 60, 70, 80, 90, 91, 92, 93, 94, 95, 96, 97, 98, 99, or more percent of the total cells in the composition or cells of a certain type such as T cells or CD8+ or CD4+ cells. Among the compositions are pharmaceutical compositions and formulations for administration, such as for adoptive cell therapy. Also provided are therapeutic methods for administering the cells and compositions to subjects, e.g., patients.

Thus also provided are genetically engineered cells expressing the recombinant receptors containing the antibodies, e.g., cells containing the CARs. The cells generally are eukaryotic cells, such as mammalian cells, and typically are human cells. In some embodiments, the cells are derived from the blood, bone marrow, lymph, or lymphoid organs, are cells of the immune system, such as cells of the innate or adaptive immunity, e.g., myeloid or lymphoid cells, including lymphocytes, typically T cells and/or NK cells. Other exemplary cells include stem cells, such as multipotent and pluripotent stem cells, including induced pluripotent stem cells (iPSCs). The cells typically are primary cells, such as those isolated directly from a subject and/or isolated from a subject and frozen. In some embodiments, the cells include one or more subsets of T cells or other cell types, such as whole T cell populations, CD4′ cells, CD8⁺ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. With reference to the subject to be treated, the cells may be allogeneic and/or autologous. Among the methods include off-the-shelf methods. In some aspects, such as for off-the-shelf technologies, the cells are pluripotent and/or multipotent, such as stem cells, such as induced pluripotent stem cells (iPSCs). In some embodiments, the methods include isolating cells from the subject, preparing, processing, culturing, and/or engineering them, as described herein, and re-introducing them into the same patient, before or after cryopreservation.

Among the sub-types and subpopulations of T cells and/or of CD4⁺ and/or of CD8⁺ T cells are naïve T (T_{N}) cells, effector T cells (T_{EFF}), memory T cells and sub-types thereof, such as stem cell memory T (T_{SCM}), central memory T (T_{CM}), effector memory T (T_{EM}), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells.

In some embodiments, the cells are natural killer (NK) cells. In some embodiments, the cells are monocytes or granulocytes, e.g., myeloid cells, macrophages, neutrophils, dendritic cells, mast cells, eosinophils, and/or basophils.

In some embodiments, the cells include one or more polynucleotides introduced via genetic engineering, and thereby express recombinant or genetically engineered products of such polynucleotides. In some embodiments, the polynucleotides are heterologous, i.e., normally not present in a cell or sample obtained from the cell, such as one obtained from another organism or cell, which for example, is not ordinarily found in the cell being engineered and/or an organism from which such cell is derived. In some embodiments, the polynucleotides are not naturally occurring, such as a polynucleotide not found in nature, including one comprising chimeric combinations of polynucleotides encoding various domains from multiple different cell types. In some embodiments, the cells (e.g., engineered cells) comprise a vector (e.g., a viral vector, expression vector, etc.) as described herein such as a vector comprising a nucleic acid encoding a recombinant receptor described herein.

### A. Vectors and Methods for Genetic Engineering

Also provided are nucleic acids, e.g., polynucleotides, encoding the antibodies and/or portions, e.g., chains, thereof. Among the provided nucleic acids are those encoding the anti-ROR1 antibodies (e.g., antigen-binding fragment) described herein. Also provided are nucleic acids, e.g., polynucleotides, encoding one or more antibodies and/or portions thereof, e.g., those encoding one or more of the anti-ROR1 antibodies (e.g., antigen-binding fragment) described herein and/or other antibodies and/or portions thereof, e.g., antibodies and/or portions thereof that binds other target antigens. Also provided are methods, nucleic acids, compositions, and kits, for expressing the binding molecules (e.g., anti-ROR1 binding molecules), including recombinant receptors (e.g., CARs) comprising the binding molecules, and for producing the genetically engineered cells expressing such binding molecules. In some embodiments, one or more binding molecules, including recombinant receptors (e.g., CARs) can be genetically engineered into cells or plurality of cells. The genetic engineering generally involves introduction of a nucleic acid encoding the recombinant or engineered component into the cell, such as by retroviral transduction, transfection, or transformation.

The nucleic acids may include those encompassing natural and/or non-naturally occurring nucleotides and bases, e.g., including those with backbone modifications. The terms “nucleic acid molecule”, “nucleic acid” and “polynucleotide” may be used interchangeably, and refer to a polymer of nucleotides. Such polymers of nucleotides may contain natural and/or non-natural nucleotides, and include, but are not limited to, DNA, RNA, and PNA. “Nucleic acid sequence” refers to the linear sequence of nucleotides that comprise the nucleic acid molecule or polynucleotide. In some aspects, the nucleic acid sequence encoding at least a portion of the antibody or antigen-binding fragment thereof, such as an scFv, conjugates, receptors (e.g., CARs) provided herein can be optimized, for example, codon-optimized for expression in a human cell and/or optimized to reduce or eliminate cryptic splice sites.

In some embodiments, the polynucleotides also include one or more additional sequences, such as those encoding one or more additional molecules, such as a marker, or promoters, regulatory elements and/or multicistronic elements. In some embodiments, the provided polynucleotides include any of the polynucleotides described herein, e.g., in Section I.E.

Also provided are vectors containing the nucleic acids, e.g., polynucleotides, and host cells containing the vectors, e.g., for producing the antibodies or antigen-binding fragments thereof. Also provided are methods for producing the antibodies or antigen-binding fragments thereof. The nucleic acid may encode an amino acid sequence comprising the V_{L} region and/or an amino acid sequence comprising the V_{H} region of the antibody (e.g., the light and/or heavy chains of the antibody). The nucleic acid may encode one or more amino acid sequence comprising the V_{L} region and/or an amino acid sequence comprising the V_{H} region of the antibody (e.g., the light and/or heavy chains of the antibody). In some embodiments, the nucleic acid, e.g., polynucleotide encodes one or more V_{H} region and/or one or more V_{L} region of the antibody, in any order or orientation. In some embodiments, the nucleic acid, e.g., polynucleotide encodes a V_{H} region and a V_{L} region, and the coding sequence for the V_{H} region is upstream of the coding sequence for the V_{L} region. In some embodiments, the nucleic acid, e.g., polynucleotide encodes a V_{H} region and a V_{L} region, and the coding sequence for the V_{L} region is upstream of the coding sequence for the V_{H} region.

Also provided are vectors containing the nucleic acids, e.g., polynucleotides, and engineered cells containing the vectors, e.g., engineered immune cells expressing the binding molecules such as recombinant receptors. Also provided are methods for engineering cells, such as immune cells, to express the anti-ROR1 binding molecule, such as a recombinant receptor, e.g., a chimeric antigen receptor (CAR). The nucleic acid may encode an amino acid sequence comprising the V_{L} region and/or an amino acid sequence comprising the V_{H} region of the antibody (e.g., the light and/or heavy chains of the antibody) as the extracellular antigen-binding domain; a transmembrane domain and intracellular domains, such as a CD3 zeta and a costimulatory signaling domain.

In a further embodiment, one or more vectors (e.g., expression vectors) comprising such polynucleotides are provided. In a further embodiment, a host cell comprising such polynucleotides is provided. In one such embodiment, a host cell comprises (e.g., has been transformed with) a vector comprising a nucleic acid that encodes an amino acid sequence comprising the V_{H} region of the antibody. In another such embodiment, a host cell comprises (e.g., has been transformed with) (1) a vector comprising a nucleic acid that encodes an amino acid sequence comprising the V_{L} region of the antibody and an amino acid sequence comprising the V_{H} region of the antibody, or (2) a first vector comprising a nucleic acid that encodes an amino acid sequence comprising the V_{L} region of the antibody and a second vector comprising a nucleic acid that encodes an amino acid sequence comprising the V_{H} region of the antibody. In some embodiments, a host cell comprises (e.g., has been transformed with) one or more vectors comprising one or more nucleic acid that encodes one or more an amino acid sequence comprising one or more antibodies and/or portions thereof, e.g., antigen-binding fragments thereof. In some embodiments, one or more such host cells are provided. In some embodiments, a composition containing one or more such host cells are provided. In some embodiments, the one or more host cells can express different antibodies, or the same antibody. In some embodiments, each of the host cells can express more than one antibody.

Also provided are methods of making the anti-ROR1 chimeric antigen receptors. For recombinant production of the chimeric receptors, a nucleic acid sequence encoding a chimeric receptor antibody, e.g., as described herein, may be isolated and inserted into one or more vectors for further cloning and/or expression in a host cell. Such nucleic acid sequences may be readily isolated and sequenced using conventional procedures (e.g., by using oligonucleotide probes that are capable of binding specifically to genes encoding the heavy and light chains of the antibody). In some embodiments, a method of making the anti-ROR1 chimeric antigen receptor is provided, wherein the method comprises culturing a host cell comprising a nucleic acid sequence encoding the antibody, as provided above, under conditions suitable for expression of the receptor.

Also provided are methods of making the anti-ROR1 antibodies (including antigen-binding fragments). For recombinant production of the anti-ROR1 antibody, a nucleic acid sequence or a polynucleotide encoding an antibody, e.g., as described above, may be isolated and inserted into one or more vectors for further cloning and/or expression in a host cell. Such nucleic acid sequences may be readily isolated and sequenced using conventional procedures (e.g., by using oligonucleotide probes that are capable of binding specifically to genes encoding the heavy and light chains of the antibody). In some embodiments, a method of making the anti-ROR1 antibody is provided, wherein the method comprises culturing a host cell comprising a nucleic acid sequence encoding the antibody, as provided above, under conditions suitable for expression of the antibody, and optionally recovering the antibody from the host cell (or host cell culture medium).

In addition to prokaryotes, eukaryotic microbes such as filamentous fungi or yeast are suitable cloning or expression hosts for antibody-encoding vectors, including fungi and yeast strains whose glycosylation pathways have been modified to mimic or approximate those in human cells, resulting in the production of an antibody with a partially or fully human glycosylation pattern. See Gerngross, Nat. Biotech. 22:1409-1414 (2004), and Li et al., Nat. Biotech. 24:210-215 (2006).

Exemplary eukaryotic cells that may be used to express polypeptides include, but are not limited to, COS cells, including COS 7 cells; 293 cells, including 293-6E cells; CHO cells, including CHO-S, DG44. Lec13 CHO cells, and FUT8 CHO cells; PER.C6® cells; and NSO cells. In some embodiments, the antibody heavy chains and/or light chains (e.g., V_{H} region and/or V_{L} region) may be expressed in yeast. See, e.g., U.S. Publication No. US 2006/0270045 A1. In some embodiments, a particular eukaryotic host cell is selected based on its ability to make desired post-translational modifications to the heavy chains and/or light chains (e.g., V_{H} region and/or V_{L} region). For example, in some embodiments, CHO cells produce polypeptides that have a higher level of sialylation than the same polypeptide produced in 293 cells.

In some embodiments, the antibody or antigen-binding fragment provided herein is produced in a cell-free system. Exemplary cell-free systems are described, e.g., in Sitaraman et al., Methods Mol. Biol. 498: 229-44 (2009); Spirin, Trends Biotechnol. 22: 538-45 (2004); Endo et al., Biotechnol. Adv. 21: 695-713 (2003).

The provided embodiments further include vectors and host cells and other expression systems for expressing and producing the antibodies and other antigen-binding proteins, including eukaryotic and prokaryotic host cells, including bacteria, filamentous fungi, and yeast, as well as mammalian cells such as human cells, as well as cell-free expression systems.

In some embodiments, gene transfer is accomplished by first stimulating the cell, such as by combining it with a stimulus that induces a response such as proliferation, survival, and/or activation, e.g., as measured by expression of a cytokine or activation marker, followed by transduction of the activated cells, and expansion in culture to numbers sufficient for clinical applications.

In some contexts, overexpression of a stimulatory factor (for example, a lymphokine or a cytokine) may be toxic to a subject. Thus, in some contexts, the engineered cells include gene segments that cause the cells to be susceptible to negative selection in vivo, such as upon administration in adoptive immunotherapy. For example in some aspects, the cells are engineered so that they can be eliminated as a result of a change in the in vivo condition of the patient to which they are administered. The negative selectable phenotype may result from the insertion of a gene that confers sensitivity to an administered agent, for example, a compound. Negative selectable genes include the Herpes simplex virus type I thymidine kinase (HSV-I TK) gene (Wigler et al., Cell 2:223, 1977) which confers ganciclovir sensitivity; the cellular hypoxanthine phosphoribosyltransferase (HPRT) gene, the cellular adenine phosphoribosyltransferase (APRT) gene, bacterial cytosine deaminase, (Mullen et al., Proc. Natl. Acad. Sci. USA. 89:33 (1992)).

In some aspects, the cells further are engineered to promote expression of cytokines or other factors. Various methods for the introduction of genetically engineered components, e.g., antigen receptors, e.g., CARs, are well known and may be used with the provided methods and compositions. Exemplary methods include those for transfer of polynucleotides encoding the receptors, including via viral, e.g., retroviral or lentiviral, transduction, transposons, and electroporation.

In some embodiments, recombinant polynucleotides are transferred into cells using recombinant infectious virus particles, such as, e.g., vectors derived from simian virus 40 (SV40), adenoviruses, adeno-associated virus (AAV). In some embodiments, recombinant polynucleotides are transferred into T cells using recombinant lentiviral vectors, such as HIV-1 lentivirus-based vectors (lentivectors; see, e.g., Amado et al., Science. 1999 Jul. 30; 285(5428):674-676), or retroviral vectors, such as gamma-retroviral vectors (see, e.g., Koste et al. (2014) Gene Therapy 2014 Apr. 3. doi: 10.1038/gt.2014.25; Carlens et al. (2000) Exp Hematol 28(10): 1137-46; Alonso-Camino et al. (2013) Mol Ther Nucl Acids 2, e93; Park et al., Trends Biotechnol. 2011 Nov. 29(11): 550-557).

In some embodiments, the retroviral vector or lentiviral vector has a long terminal repeat sequence (LTR). In some embodiments the vector is derived from the Moloney murine leukemia virus (MoMLV), myeloproliferative sarcoma virus (MPSV), murine embryonic stem cell virus (MESV), murine stem cell virus (MSCV), spleen focus forming virus (SFFV), human immunodeficiency virus type 1 (HIV-1) or human immunodeficiency virus type 2 (HIV-2/SIV). In some embodiments, the vectors are self-inactivating (SIN). In some embodiments, the vectors are conditionally replicating (mobilizable) vectors. Most lentiviral vectors are derived from human, feline or simian lentiviruses. Most retroviral vectors are derived from murine retroviruses. In some embodiments, the lentiviruses or retroviruses include those derived from any avian or mammalian cell source. The lentiviruses or retroviruses typically are amphotropic, meaning that they are capable of infecting host cells of several species, including humans. In one embodiment, the gene to be expressed replaces the retroviral gag, pol and/or env sequences. Methods of lentiviral transduction are known. Exemplary methods are described in, e.g., Wang et al. (2012) J. Immunother. 35(9): 689-701; Cooper et al. (2003) Blood. 101:1637-1644; Verhoeyen et al. (2009) Methods Mol Biol. 506: 97-114; and Cavalieri et al. (2003) Blood. 102(2): 497-505. A number of illustrative retroviral systems have also been described (e.g., Amado et al., (1999) Science 285(5428):674-676, U.S. Pat. Nos. 5,219,740; 6,207,453; 5,219,740; Miller and Rosman (1989) BioTechniques 7:980-990; Miller (1990) Human Gene Therapy 1:5-14; Scarpa et al. (1991) Virology 180:849-852; Burns et al. (1993) Proc. Natl. Acad. Sci. USA 90:8033-8037; and Boris-Lawrie and Temin (1993) Cur. Opin. Genet. Develop. 3:102-109).

In some embodiments, recombinant polynucleotides are transferred into T cells via electroporation (see, e.g., Chicaybam et al, (2013) PLoS ONE 8(3): e60298 and Van Tedeloo et al. (2000) Gene Therapy 7(16): 1431-1437). In some embodiments, recombinant polynucleotides are transferred into T cells via transposition (see, e.g., Manuri et al. (2010) Hum Gene Ther 21(4): 427-437; Sharma et al. (2013) Molec Ther Nucl Acids 2, e74; and Huang et al. (2009) Methods Mol Biol 506: 115-126). Other methods of introducing and expressing genetic material in immune cells include calcium phosphate transfection (e.g., as described in Current Protocols in Molecular Biology, John Wiley & Sons, New York. N.Y.), protoplast fusion, cationic liposome-mediated transfection; tungsten particle-facilitated microparticle bombardment (Johnston (1990) Nature 346: 776-777); and strontium phosphate DNA co-precipitation (Brash et al., (1987) Mol. Cell Biol. 7: 2031-2034). Other approaches and vectors for transfer of the polynucleotides encoding the recombinant products are those described, e.g., in WO2014055668, and U.S. Pat. No. 7,446,190.

Among additional polynucleotides, e.g., genes for introduction are those to improve the outcome of therapy, such as by promoting viability and/or function of transferred cells; genes to provide a genetic marker for selection and/or evaluation of the cells, such as to assess in vivo survival or localization; genes to improve safety, for example, by making the cell susceptible to negative selection in vivo as described by Lupton S. D. et al., Mol. and Cell Biol., 11:6 (1991); and Riddell et al., Human Gene Therapy 3:319-338 (1992); see also the publications of PCT/US91/08442 and PCT/US94/05601 by Lupton et al. describing the use of bifunctional selectable fusion genes derived from fusing a dominant positive selectable marker with a negative selectable marker. See, e.g., Riddell et al., U.S. Pat. No. 6,040,177, at columns 14-17.

In some embodiments the vector or construct can contain a promoter and/or enhancer or regulatory elements to regulate expression of the encoded recombinant receptor. In some examples the promoter and/or enhancer or regulatory elements can be condition-dependent promoters, enhancers, and/or regulatory elements. In some examples these elements drive expression of the transgene. In some examples, the CAR transgene can be operatively linked to a promoter, such as an EF1alpha promoter with an HTLV1 enhancer (SEQ ID NO:164). In some examples, the CAR transgene is operatively linked to a Woodchuck Hepatitis Virus (WHP) Posttranscriptional Regulatory Element (WPRE; SEQ ID NO: 165), located downstream of the transgene.

In some embodiments, the vector or construct can contain a single promoter that drives the expression of one or more nucleic acid molecules. In some embodiments, such nucleic acid molecules, e.g., transcripts, can be multicistronic (bicistronic or tricistronic, see e.g., U.S. Pat. No. 6,060,273). For example, in some embodiments, transcription units can be engineered as a bicistronic unit containing an IRES (internal ribosome entry site), which allows coexpression of gene products (e.g., encoding a first and second chimeric receptor) by a message from a single promoter. For example, in some embodiments, the vector or construct can contain a nucleic acid encoding an anti-ROR1 receptor (e.g., an anti-ROR1 CAR) provided herein and a nucleic acid encoding a different molecule, separated by an IRES, under the regulation of a single promoter.

Alternatively, in some cases, a single promoter may direct expression of an RNA that contains, in a single open reading frame (ORF), two or three genes (e.g. encoding a first and second binding molecules, e.g., antibody recombinant receptor) separated from one another by sequences encoding a self-cleavage peptide (e.g., 2A cleavage sequences) or a protease recognition site (e.g., furin). The ORF thus encodes a single polypeptide, which, either during (in the case of T2A) or after translation, is cleaved into the individual proteins. In some cases, the peptide, such as T2A, can cause the ribosome to skip (ribosome skipping) synthesis of a peptide bond at the C-terminus of a 2A element, leading to separation between the end of the 2A sequence and the next peptide downstream (see, for example, de Felipe. Genetic Vaccines and Ther. 2:13 (2004) and deFelipe et al. Traffic 5:616-626 (2004)). Many 2A elements are known. Examples of 2A sequences that can be used in the methods and polynucleotides disclosed herein, without limitation, 2A sequences from the foot-and-mouth disease virus (F2A, e.g., SEQ ID NO:21 or 168), equine rhinitis A virus (E2A, e.g., SEQ ID NO: 20 or 167), Thosea asigna virus (T2A, e.g., SEQ ID NO: 6, 17 or 166), and porcine teschovirus-1 (P2A, e.g., SEQ ID NO: 18 or 19) as described in U.S. Patent Publication No. 20070116690. In some embodiments, the one or more different or separate promoters drive the expression of one or more nucleic acid molecules encoding the one or more binding molecules, e.g., recombinant receptors.

Any of the recombinant receptors provided herein, e.g., anti-ROR1 recombinant receptors and/or the additional recombinant receptors, can be encoded by polynucleotides containing one or more nucleic acid molecules encoding the receptors, in any combinations or arrangements. For example, one, two, three or more polynucleotides can encode one, two, three or more different receptors or domains. In some embodiments, one vector or construct contains nucleic acid molecules encoding one or more recombinant receptor(s), and a separate vector or construct contains nucleic acid molecules encoding an additional binding molecule, e.g., antibody and/or recombinant receptor, such as an anti-ROR1 receptor (e.g., anti-ROR1 CAR).

In some embodiments, the nucleic acid molecules can also encode one or more surrogate marker(s), such as fluorescent protein (e.g., green fluorescent protein (GFP)) or a cell surface marker (e.g., a truncated surface marker such as truncated EGFR (tEGFR), which may be used to confirm transduction or engineering of the cell to express the receptor. For example, in some aspects, extrinsic marker genes are utilized in connection with engineered cell therapies to permit detection or selection of cells and, in some cases, also to promote cell suicide by ADCC. Exemplary marker genes include truncated epidermal growth factor receptor (EGFRt), which can be co-expressed with a transgene of interest (e.g., a CAR or TCR) in transduced cells (see, e.g., U.S. Pat. No. 8,802,374). EGFRt contains an epitope recognized by the antibody cetuximab (Erbitux®). For this reason, Erbitux® can be used to identify or select cells that have been engineered with the EGFRt construct, including in cells also co-engineered with another recombinant receptor, such as a chimeric antigen receptor (CAR).

In some embodiments, the nucleic acid encoding the binding molecules further contain contains a nucleic acid sequence encoding one or more marker(s). In some embodiments, the one or more marker(s) is a transduction marker, surrogate marker and/or a selection marker.

In some embodiments, the marker is a transduction marker or a surrogate marker. A transduction marker or a surrogate marker can be used to detect cells that have been introduced with the polynucleotide, e.g., a polynucleotide encoding a recombinant receptor. In some embodiments, the transduction marker can indicate or confirm modification of a cell. In some embodiments, the surrogate marker is a protein that is made to be co-expressed on the cell surface with the recombinant receptor, e.g. CAR. In particular embodiments, such a surrogate marker is a surface protein that has been modified to have little or no activity. In certain embodiments, the surrogate marker is encoded on the same polynucleotide that encodes the recombinant receptor. In some embodiments, the nucleic acid sequence encoding the recombinant receptor is operably linked to a nucleic acid sequence encoding a marker, optionally separated by an internal ribosome entry site (IRES), or a nucleic acid encoding a self-cleaving peptide or a peptide that causes ribosome skipping, such as a 2A sequence, such as a T2A, a P2A, an E2A or an F2A. Extrinsic marker genes may in some cases be utilized in connection with engineered cell to permit detection or selection of cells and, in some cases, also to promote cell suicide.

Exemplary surrogate markers can include truncated forms of cell surface polypeptides, such as truncated forms that are non-functional and to not transduce or are not capable of transducing a signal or a signal ordinarily transduced by the full-length form of the cell surface polypeptide, and/or do not or are not capable of internalizing. Exemplary truncated cell surface polypeptides including truncated forms of growth factors or other receptors such as a truncated human epidermal growth factor receptor 2 (tHER2), a truncated epidermal growth factor receptor (tEGFR, exemplary tEGFR sequence set forth in SEQ ID NO:7, 16 or 153) or a prostate-specific membrane antigen (PSMA) or modified form thereof. tEGFR may contain an epitope recognized by the antibody cetuximab (Erbitux®) or other therapeutic anti-EGFR antibody or binding molecule, which can be used to identify or select cells that have been engineered with the tEGFR construct and an encoded exogenous protein, and/or to eliminate or separate cells expressing the encoded exogenous protein. See U.S. Pat. No. 8,802,374 and Liu et al., Nature Biotech. 2016 April; 34(4): 430-434). In some aspects, the marker, e.g. surrogate marker, includes all or part (e.g., truncated form) of CD34, a NGFR, a CD19 or a truncated CD19, e.g., a truncated non-human CD19, or epidermal growth factor receptor (e.g., tEGFR).

In some embodiments, the marker is or comprises a fluorescent protein, such as green fluorescent protein (GFP), enhanced green fluorescent protein (EGFP), such as super-fold GFP (sfGFP), red fluorescent protein (RFP), such as tdTomato, mCherry, mStrawberry, AsRed2, DsRed or DsRed2, cyan fluorescent protein (CFP), blue green fluorescent protein (BFP), enhanced blue fluorescent protein (EBFP), and yellow fluorescent protein (YFP), and variants thereof, including species variants, monomeric variants, and codon-optimized and/or enhanced variants of the fluorescent proteins. In some embodiments, the marker is or comprises an enzyme, such as a luciferase, the lacZ gene from E. coli, alkaline phosphatase, secreted embryonic alkaline phosphatase (SEAP), chloramphenicol acetyl transferase (CAT). Exemplary light-emitting reporter genes include luciferase (luc), β-galactosidase, chloramphenicol acetyltransferase (CAT), β-glucuronidase (GUS) or variants thereof.

In some embodiments, the marker is a selection marker. In some embodiments, the selection marker is or comprises a polypeptide that confers resistance to exogenous agents or drugs. In some embodiments, the selection marker is an antibiotic resistance gene. In some embodiments, the selection marker is an antibiotic resistance gene confers antibiotic resistance to a mammalian cell. In some embodiments, the selection marker is or comprises a Puromycin resistance gene, a Hygromycin resistance gene, a Blasticidin resistance gene, a Neomycin resistance gene, a Geneticin resistance gene or a Zeocin resistance gene or a modified form thereof.

In some embodiments, the nucleic acid encoding the marker is operably linked to a polynucleotide encoding for a linker sequence, such as a cleavable linker sequence, e.g., a T2A. For example, a marker, and optionally a linker sequence, can be any as disclosed in PCT Pub. No.

WO2014031687. For example, the marker can be a truncated EGFR (tEGFR) that is, optionally, linked to a linker sequence, such as a T2A cleavable linker sequence. An exemplary polypeptide for a truncated EGFR (e.g. tEGFR) comprises the sequence of amino acids set forth in SEQ ID NO: 7, 16 or 153 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 7, 16 or 153.

In some embodiments, the marker is a molecule, e.g., cell surface protein, not naturally found on T cells or not naturally found on the surface of T cells, or a portion thereof.

In some embodiments, the molecule is a non-self molecule, e.g., non-self protein, i.e., one that is not recognized as “self” by the immune system of the host into which the cells will be adoptively transferred.

In some embodiments, the marker serves no therapeutic function and/or produces no effect other than to be used as a marker for genetic engineering, e.g., for selecting cells successfully engineered.

In other embodiments, the marker may be a therapeutic molecule or molecule otherwise exerting some desired effect, such as a ligand for a cell to be encountered in vivo, such as a costimulatory or immune checkpoint molecule to enhance and/or dampen responses of the cells upon adoptive transfer and encounter with ligand.

Also provided are compositions containing one or more of the nucleic acid molecules, vectors or constructs, such as any described above. In some embodiments, the nucleic acid molecules, vectors, constructs or compositions can be used to engineer cells, such as T cells, to express any of the binding molecules, e.g., antibody or recombinant receptor, and/or the additional binding molecules.

### B. Preparation of Cells for Engineering

In some embodiments, preparation of the engineered cells includes one or more culture and/or preparation steps. The cells for introduction of the recombinant receptor (e.g., CAR) may be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered.

Accordingly, the cells in some embodiments are primary cells, e.g., primary human cells. The samples include tissue, fluid, and other samples taken directly from the subject, as well as samples resulting from one or more processing steps, such as separation, centrifugation, genetic engineering (e.g., transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample from which the cells are derived or isolated is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some embodiments, the cells are derived from cell lines, e.g., T cell lines. The cells in some embodiments are obtained from a xenogeneic source, for example, from mouse, rat, non-human primate, or pig.

In some embodiments, isolation of the cells includes one or more preparation and/or non-affinity based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components.

In some examples, cells from the circulating blood of a subject are obtained, e.g., by apheresis or leukapheresis. The samples, in some aspects, contain lymphocytes, including T cells, monocytes, granulocytes, B cells, other nucleated white blood cells, red blood cells, and/or platelets, and in some aspects contain cells other than red blood cells and platelets.

In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some embodiments, the wash solution lacks calcium and/or magnesium and/or many or all divalent cations. In some aspects, a washing step is accomplished a semi-automated “flow-through” centrifuge (for example, the Cobe 2991 cell processor, Baxter) according to the manufacturer's instructions. In some aspects, a washing step is accomplished by tangential flow filtration (TFF) according to the manufacturer's instructions. In some embodiments, the cells are resuspended in a variety of biocompatible buffers after washing, such as, for example, Ca++/Mg++ free PBS. In certain embodiments, components of a blood cell sample are removed and the cells directly resuspended in culture media.

In some embodiments, the methods include density-based cell separation methods, such as the preparation of white blood cells from peripheral blood by lysing the red blood cells and centrifugation through a Percoll or Ficoll gradient.

In some embodiments, the isolation methods include the separation of different cell types based on the expression or presence in the cell of one or more specific molecules, such as surface markers, e.g., surface proteins, intracellular markers, or nucleic acid. In some embodiments, any known method for separation based on such markers may be used. In some embodiments, the separation is affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps can be based on positive selection, in which the cells having bound the reagents are retained for further use, and/or negative selection, in which the cells having not bound to the antibody or binding partner are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population.

The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In some examples, multiple rounds of separation steps are carried out, where the positively or negatively selected fraction from one step is subjected to another separation step, such as a subsequent positive or negative selection. In some examples, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types.

For example, in some aspects, specific subpopulations of T cells, such as cells positive or expressing high levels of one or more surface markers, e.g., CD28+, CD62L+, CCR7+, CD27+, CD127+, CD4⁺, CD8⁺, CD45RA+, and/or CD45RO+ T cells, are isolated by positive or negative selection techniques.

For example, CD3⁺, CD28+ T cells can be positively selected using anti-CD3/anti-CD28 conjugated magnetic beads (e.g., DYNABEADS® M-450 CD3/CD28 T Cell Expander, MACSiBeads™ etc.).

In some embodiments, isolation is carried out by enrichment for a particular cell population by positive selection, or depletion of a particular cell population, by negative selection. In some embodiments, positive or negative selection is accomplished by incubating cells with one or more antibodies or other binding agent that specifically bind to one or more surface markers expressed or expressed (marker+) at a relatively higher level (marker^{high}) on the positively or negatively selected cells, respectively.

In some embodiments, T cells are separated from a PBMC sample by negative selection of markers expressed on non-T cells, such as B cells, monocytes, or other white blood cells, such as CD14. In some aspects, a CD4+ or CD8+ selection step is used to separate CD4+ helper and CD8+ cytotoxic T cells. Such CD4+ and CD8+ populations can be further sorted into sub-populations by positive or negative selection for markers expressed or expressed to a relatively higher degree on one or more naive, memory, and/or effector T cell subpopulations.

In some embodiments, CD8+ cells are further enriched for or depleted of naive, central memory, effector memory, and/or central memory stem cells, such as by positive or negative selection based on surface antigens associated with the respective subpopulation. In some embodiments, enrichment for central memory T (T_{CM}) cells is carried out to increase certain features, such as to improve long-term survival, expansion, and/or engraftment following administration, which in some aspects is particularly robust in such sub-populations (see Terakura et al. (2012) Blood. 1:72-82; Wang et al. (2012) J Immunother. 35(9):689-701). In some embodiments, combining TCM-enriched CD8+ T cells and CD4+ T cells further enhances response.

In embodiments, memory T cells are present in both CD62L+ and CD62− subsets of CD8+ peripheral blood lymphocytes. PBMC can be enriched for or depleted of CD62L-CD8+ and/or CD62L+CD8+ fractions, such as using anti-CD8 and anti-CD62L antibodies.

In some embodiments, the enrichment for central memory T (T_{CM}) cells is based on positive or high surface expression of CD45RO, CD62L, CCR7, CD28, CD3, and/or CD 127; in some aspects, it is based on negative selection for cells expressing or highly expressing CD45RA and/or granzyme B. In some aspects, isolation of a CD8+ population enriched for TCM cells is carried out by depletion of cells expressing CD4, CD14, CD45RA, and positive selection or enrichment for cells expressing CD62L. In one aspect, enrichment for central memory T (T_{CM}) cells is carried out starting with a negative fraction of cells selected based on CD4 expression, which is subjected to a negative selection based on expression of CD14 and CD45RA, and a positive selection based on CD62L. Such selections in some aspects are carried out simultaneously and in other aspects are carried out sequentially, in either order. In some aspects, the same CD4 expression-based selection step used in preparing the CD8+ cell population or subpopulation, also is used to generate the CD4+ cell population or sub-population, such that both the positive and negative fractions from the CD4-based separation are retained and used in subsequent steps of the methods, optionally following one or more further positive or negative selection steps.

In a particular example, a sample of PBMCs or other white blood cell sample is subjected to selection of CD4+ cells, where both the negative and positive fractions are retained. The negative fraction then is subjected to negative selection based on expression of CD14 and CD45RA, and positive selection based on a marker characteristic of central memory T cells, such as CD62L or CCR7, where the positive and negative selections are carried out in either order.

CD4+ T helper cells are sorted into naïve, central memory, and effector cells by identifying cell populations that have cell surface antigens. CD4+ lymphocytes can be obtained by standard methods. In some embodiments, naive CD4+ T lymphocytes are CD45RO−, CD45RA+, CD62L+, CD4+ T cells. In some embodiments, central memory CD4+ cells are CD62L+ and CD45RO+. In some embodiments, effector CD4+ cells are CD62L− and CD45RO−.

In one example, to enrich for CD4+ cells by negative selection, a monoclonal antibody cocktail typically includes antibodies to CD14, CD20, CD11b, CD16, HLA-DR, and CD8. In some embodiments, the antibody or binding partner is bound to a solid support or matrix, such as a magnetic bead or paramagnetic bead, to allow for separation of cells for positive and/or negative selection. For example, in some embodiments, the cells and cell populations are separated or isolated using immunomagnetic (or affinity magnetic) separation techniques (reviewed in Methods in Molecular Medicine, vol. 58: Metastasis Research Protocols, Vol. 2: Cell Behavior In vitro and In vivo, p 17-25 Edited by: S. A. Brooks and U. Schumacher © Humana Press Inc., Totowa, NJ).

In some aspects, the sample or composition of cells to be separated is incubated with small, magnetizable or magnetically responsive material, such as magnetically responsive particles or microparticles, such as paramagnetic beads (e.g., such as Dynabeads® or MACS® beads). The magnetically responsive material, e.g., particle, generally is directly or indirectly attached to a binding partner, e.g., an antibody, that specifically binds to a molecule, e.g., surface marker, present on the cell, cells, or population of cells that it is desired to separate, e.g., that it is desired to negatively or positively select.

In some embodiments, the magnetic particle or bead comprises a magnetically responsive material bound to a specific binding member, such as an antibody or other binding partner. There are many well-known magnetically responsive materials used in magnetic separation methods. Suitable magnetic particles include those described in Molday, U.S. Pat. No. 4,452,773, and in European Patent Specification EP 452342 B, which are hereby incorporated by reference. Colloidal sized particles, such as those described in Owen U.S. Pat. No. 4,795,698, and Liberti et al., U.S. Pat. No. 5,200,084, are other examples.

The incubation generally is carried out under conditions whereby the antibodies or binding partners, or molecules, such as secondary antibodies or other reagents, which specifically bind to such antibodies or binding partners, which are attached to the magnetic particle or bead, specifically bind to cell surface molecules if present on cells within the sample.

In some aspects, the sample is placed in a magnetic field, and those cells having magnetically responsive or magnetizable particles attached thereto will be attracted to the magnet and separated from the unlabeled cells. For positive selection, cells that are attracted to the magnet are retained; for negative selection, cells that are not attracted (unlabeled cells) are retained. In some aspects, a combination of positive and negative selection is performed during the same selection step, where the positive and negative fractions are retained and further processed or subject to further separation steps.

In certain embodiments, the magnetically responsive particles are coated in primary antibodies or other binding partners, secondary antibodies, lectins, enzymes, or streptavidin. In certain embodiments, the magnetic particles are attached to cells via a coating of primary antibodies specific for one or more markers. In certain embodiments, the cells, rather than the beads, are labeled with a primary antibody or binding partner, and then cell-type specific secondary antibody- or other binding partner (e.g., streptavidin)-coated magnetic particles, are added. In certain embodiments, streptavidin-coated magnetic particles are used in conjunction with biotinylated primary or secondary antibodies.

In some embodiments, the magnetically responsive particles are left attached to the cells that are to be subsequently incubated, cultured and/or engineered; in some aspects, the particles are left attached to the cells for administration to a patient. In some embodiments, the magnetizable or magnetically responsive particles are removed from the cells. Methods for removing magnetizable particles from cells are known and include, e.g., the use of competing non-labeled antibodies, magnetizable particles or antibodies conjugated to cleavable linkers, etc. In some embodiments, the magnetizable particles are biodegradable.

In some embodiments, the affinity-based selection is via magnetic-activated cell sorting (MACS®) (Miltenyi Biotec, Auburn, CA). Magnetic Activated Cell Sorting (MACS®) systems are capable of high-purity selection of cells having magnetized particles attached thereto. In certain embodiments, MACS® operates in a mode wherein the non-target and target species are sequentially eluted after the application of the external magnetic field. That is, the cells attached to magnetized particles are held in place while the unattached species are eluted. Then, after this first elution step is completed, the species that were trapped in the magnetic field and were prevented from being eluted are freed in some manner such that they can be eluted and recovered. In certain embodiments, the non-target cells are labelled and depleted from the heterogeneous population of cells.

In certain embodiments, the isolation or separation is carried out using a system, device, or apparatus that carries out one or more of the isolation, cell preparation, separation, processing, incubation, culture, and/or formulation steps of the methods. In some aspects, the system is used to carry out each of these steps in a closed or sterile environment, for example, to minimize error, user handling and/or contamination. In one example, the system is a system as described in WO2009/072003 or US 20110003380.

In some embodiments, the system or apparatus carries out one or more, e.g., all, of the isolation, processing, engineering, and formulation steps in an integrated or self-contained system, and/or in an automated or programmable fashion. In some aspects, the system or apparatus includes a computer and/or computer program in communication with the system or apparatus, which allows a user to program, control, assess the outcome of, and/or adjust various aspects of the processing, isolation, engineering, and formulation steps.

In some aspects, the separation and/or other steps is carried out using CliniMACS® system (Miltenyi Biotec), for example, for automated separation of cells on a clinical-scale level in a closed and sterile system. Components can include an integrated microcomputer, magnetic separation unit, peristaltic pump, and various pinch valves. The integrated computer in some aspects controls all components of the instrument and directs the system to perform repeated procedures in a standardized sequence. The magnetic separation unit in some aspects includes a movable permanent magnet and a holder for the selection column. The peristaltic pump controls the flow rate throughout the tubing set and, together with the pinch valves, ensures the controlled flow of buffer through the system and continual suspension of cells.

The CliniMACS® system in some aspects uses antibody-coupled magnetizable particles that are supplied in a sterile, non-pyrogenic solution. In some embodiments, after labelling of cells with magnetic particles the cells are washed to remove excess particles. A cell preparation bag is then connected to the tubing set, which in turn is connected to a bag containing buffer and a cell collection bag. The tubing set consists of pre-assembled sterile tubing, including a pre-column and a separation column, and are for single use only. After initiation of the separation program, the system automatically applies the cell sample onto the separation column. Labelled cells are retained within the column, while unlabeled cells are removed by a series of washing steps. In some embodiments, the cell populations for use with the methods described herein are unlabeled and are not retained in the column. In some embodiments, the cell populations for use with the methods described herein are labeled and are retained in the column. In some embodiments, the cell populations for use with the methods described herein are eluted from the column after removal of the magnetic field, and are collected within the cell collection bag.

In certain embodiments, separation and/or other steps are carried out using the CliniMACS Prodigy® system (Miltenyi Biotec). The CliniMACS Prodigy® system in some aspects is equipped with a cell processing unity that permits automated washing and fractionation of cells by centrifugation. The CliniMACS Prodigy® system can also include an onboard camera and image recognition software that determines the optimal cell fractionation endpoint by discerning the macroscopic layers of the source cell product. For example, peripheral blood may be automatically separated into erythrocytes, white blood cells and plasma layers. The CliniMACS Prodigy® system can also include an integrated cell cultivation chamber which accomplishes cell culture protocols such as, e.g., cell differentiation and expansion, antigen loading, and long-term cell culture. Input ports can allow for the sterile removal and replenishment of media and cells can be monitored using an integrated microscope (see, e.g., Klebanoff et al. (2012) J Immunother. 35(9): 651-660, Terakura et al. (2012) Blood. 1:72-82, and Wang et al. (2012) J Immunother. 35(9):689-701).

In some embodiments, a cell population described herein is collected and enriched (or depleted) via flow cytometry, in which cells stained for multiple cell surface markers are carried in a fluidic stream. In some embodiments, a cell population described herein is collected and enriched (or depleted) via preparative scale (FACS)-sorting. In certain embodiments, a cell population described herein is collected and enriched (or depleted) by use of microelectromechanical systems (MEMS) chips in combination with a FACS-based detection system (see, e.g., WO 2010/033140, Cho et al. (2010) Lab Chip 10, 1567-1573; and Godin et al. (2008) J Biophoton. 1(5):355-376. In both cases, cells can be labeled with multiple markers, allowing for the isolation of well-defined T cell subsets at high purity.

In some embodiments, the antibodies or binding partners are labeled with one or more detectable marker, to facilitate separation for positive and/or negative selection. For example, separation may be based on binding to fluorescently labeled antibodies. In some examples, separation of cells based on binding of antibodies or other binding partners specific for one or more cell surface markers are carried in a fluidic stream, such as by fluorescence-activated cell sorting (FACS), including preparative scale (FACS) and/or microelectromechanical systems (MEMS) chips, e.g., in combination with a flow-cytometric detection system. Such methods allow for positive and negative selection based on multiple markers simultaneously.

In some embodiments, the preparation methods include steps for freezing, e.g., cryopreserving, the cells, either before or after isolation, incubation, and/or engineering. In some embodiments, the freeze and subsequent thaw step removes granulocytes and, to some extent, monocytes in the cell population. In some embodiments, the cells are suspended in a freezing solution, e.g., following a washing step to remove plasma and platelets. Any of a variety of known freezing solutions and parameters in some aspects may be used. One example involves using PBS containing 20% DMSO and 8% human serum albumin (HSA), or other suitable cell freezing media. This is then diluted 1:1 with media so that the final concentration of DMSO and HSA are 10% and 4%, respectively. The cells are then frozen to −80° C. at a rate of 1° C. per minute and stored in the vapor phase of a liquid nitrogen storage tank.

In some embodiments, the provided methods include cultivation, incubation, culture, and/or genetic engineering steps. For example, in some embodiments, provided are methods for incubating and/or engineering the depleted cell populations and culture-initiating compositions.

Thus, in some embodiments, the cell populations are incubated in a culture-initiating composition. The incubation and/or engineering may be carried out in a culture vessel, such as a unit, chamber, well, column, tube, tubing set, valve, vial, culture dish, bag, or other container for culture or cultivating cells.

In some embodiments, the cells are incubated and/or cultured prior to or in connection with genetic engineering. The incubation steps can include culture, cultivation, stimulation, activation, and/or propagation. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a recombinant antigen receptor.

The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some embodiments, the stimulating conditions or agents include one or more agent, e.g., ligand, which is capable of stimulating or activating an intracellular signaling domain of a TCR complex. In some aspects, the agent turns on or initiates TCR/CD3 intracellular signaling cascade in a T cell. Such agents can include antibodies, such as those specific for a TCR component and/or costimulatory receptor, e.g., anti-CD3, anti-CD28, for example, bound to solid support such as a bead, and/or one or more cytokines. Optionally, the expansion method may further comprise the step of adding anti-CD3 and/or anti-CD28 antibody to the culture medium (e.g., at a concentration of at least about 0.5 ng/mL). In some embodiments, the stimulating agents include IL-2 and/or IL-15, for example, an IL-2 concentration of at least about 10 units/mL.

In some aspects, incubation is carried out in accordance with techniques such as those described in U.S. Pat. No. 6,040,177 to Riddell et al., Klebanoff et al. (2012) J Immunother. 35(9): 651-660, Terakura et al. (2012) Blood. 1:72-82, and/or Wang et al. (2012) J Immunother. 35(9):689-701.

In some embodiments, the T cells are expanded by adding to the culture-initiating composition feeder cells, such as non-dividing peripheral blood mononuclear cells (PBMC), (e.g., such that the resulting population of cells contains at least about 5, 10, 20, or 40 or more PBMC feeder cells for each T lymphocyte in the initial population to be expanded); and incubating the culture (e.g. for a time sufficient to expand the numbers of T cells). In some aspects, the non-dividing feeder cells can comprise gamma-irradiated PBMC feeder cells. In some embodiments, the PBMC are irradiated with gamma rays in the range of about 3000 to 3600 rads to prevent cell division. In some aspects, the feeder cells are added to culture medium prior to the addition of the populations of T cells.

In some embodiments, the stimulating conditions include temperature suitable for the growth of human T lymphocytes, for example, at least about 25 degrees Celsius, generally at least about 30 degrees, and generally at or about 37 degrees Celsius. Optionally, the incubation may further comprise adding non-dividing EBV-transformed lymphoblastoid cells (LCL) as feeder cells. LCL can be irradiated with gamma rays in the range of about 6000 to 10,000 rads. The LCL feeder cells in some aspects is provided in any suitable amount, such as a ratio of LCL feeder cells to initial T lymphocytes of at least about 10:1.

In embodiments, antigen-specific T cells, such as antigen-specific CD4+ and/or CD8+ T cells, are obtained by stimulating naive or antigen specific T lymphocytes with antigen. For example, antigen-specific T cell lines or clones can be generated to cytomegalovirus antigens by isolating T cells from infected subjects and stimulating the cells in vitro with the same antigen.

### C. Engineered Cells, Vectors and Compositions for Multi-Targeting

Also provided are cells such as engineered cells that can bind to and/or target multiple antigens. In some embodiments, improved selectivity and specificity is achieved through strategies targeting multiple antigens. Such strategies generally involve multiple antigen-binding domains, which typically are present on distinct genetically engineered antigen receptors and specifically bind to distinct antigens. In some embodiments, the cells are engineered with the ability to bind more than one antigen. For example, in some embodiments, the cells are engineered to express multispecific binding molecules. In some embodiments, the cells express multiple binding molecules, e.g., recombinant receptors, each of which can target one antigen or multiple antigens, e.g., one receptor that targets ROR1, such as any described herein, and another receptor that targets another antigen, e.g., tumor antigen. In some aspects, a plurality of genetically engineered antigen receptors are introduced into the cell, which specifically bind to different antigens, each expressed in or on the disease or condition to be targeted with the cells or tissues or cells thereof. Such features can in some aspects address or reduce the likelihood of off-target effects or increase efficacy. For example, where a single antigen expressed in a disease or condition is also expressed on or in non-diseased or normal cells, such multi-targeting approaches can provide selectivity for desired cell types by requiring binding via multiple antigen receptors in order to activate the cell or induce a particular effector function. In some embodiments, a plurality of cells can be engineered to express one or more different binding molecules, e.g., recombinant receptors, each of which can target one antigen or multiple antigens.

Also provided are multispecific cells containing any of the binding molecules described herein, such as cells containing a cell surface protein including the anti-ROR1 antibody or an antigen-binding fragment thereof and an additional cell surface protein, such as an additional chimeric receptor, which binds to a different antigen or a different epitope on ROR1. In some embodiments, provided are compositions of cells that express recombinant receptors, wherein one or more of the binding molecules, multispecific binding molecules and/or recombinant receptors bind and/or target ROR1. Also provided are compositions of cells containing a plurality of cells that express one or more different binding molecules, e.g., recombinant receptors that can target one or multiple antigens. In some embodiments, the multispecific binding molecules and/or recombinant receptors target one or more different epitopes on ROR1.

In some embodiments, provided are composition of cells, wherein each type of cell expresses one or more binding molecules, e.g., recombinant receptors. In some embodiments, the cell comprises (e.g., has been transformed with) one or more vectors comprising one or more nucleic acid that encodes one or more an amino acid sequence comprising one or more antibodies and/or portions thereof, e.g., antigen-binding fragments thereof. In some embodiments, one or more such cells are provided. In some embodiments, a composition containing one or more such cells is provided. In some embodiments, the one or more cells can express different antibodies, or the same antibody. In some embodiments, each of the cells expresses one or more antibodies, such as more than one antibody. In some embodiments, each of the cells expresses a multispecific binding molecule, e.g., a multispecific receptor, e.g., CAR.

In some embodiments, the cells include multi-targeting strategies that target ROR1 and a second or additional antigen associated with a particular disease or condition. In some embodiments, the second or additional antigen is targeted by a multispecific binding molecule and/or multiple binding molecules and/or a plurality of cells, e.g., one or more cells, each engineered to express one or more recombinant receptors. In some embodiments, a recombinant receptor targeting a second or additional antigen is expressed on the same cell as a ROR1 binding molecule, or on a different cell.

In some embodiments, among the second or additional antigens for multi-targeting strategies includes those in which at least one of the antigens is a universal tumor antigen, or a family member thereof. In some embodiments, the second or additional antigen is an antigen expressed on a tumor. In some embodiments, the ROR1-binding molecules provided herein target an antigen on the same tumor type as the second or additional antigen. In some embodiments, the second or additional antigen may also be a universal tumor antigen or may be a tumor antigen specific to a tumor type. In some embodiments, the cell further comprises an additional genetically engineered antigen receptor that recognizes a second or additional antigen expressed on a disease or condition to be treated and induces a stimulatory or activating signal.

Exemplary antigens include CD4, CD5, CD8, CD14, CD15, CD19, CD20, CD21, CD22, CD23, CD25, CD33, CD37, CD38, CD40, CD40L, CD46, CD52, CD54, CD74, CD80, CD126, CD138, B7, MUC-1, Ia, HM1.24, HLA-DR, tenascin, an angiogenesis factor, VEGF, PIGF, ED-B fibronectin, an oncogene, an oncogene product, CD66a-d, necrosis antigens, I1, IL-2, T101, TAC, IL-6, TRAIL-R1 (DR4), TRAIL-R2 (DR5), B cell maturation antigen (BCMA), Her2, L1-CAM, mesothelin, CEA, hepatitis B surface antigen, anti-folate receptor, CD24, CD30, CD44, EGFR, EGP-2, EGP-4, EPHa2, ErbB2, ErbB3, ErbB4, erbB dimers, EGFR vIII, FBP, FCRL5, FCRH5, fetal acetylcholine receptor, GD2, GD3, HMW-MAA, IL-22R-alpha, IL-13R-alpha2, kdr, kappa light chain, Lewis Y, L1-cell adhesion molecule (L1-CAM), Melanoma-associated antigen (MAGE)-A1, MAGE-A3, MAGE-A6, Preferentially expressed antigen of melanoma (PRAME), survivin, EGP2, EGP40, TAG72, B7-H6, IL-13 receptor a2 (IL-13Ra2), CA9, CD171, G250/CAIX, HLA-A1 MAGE A1, HLA-A2 NY-ESO-1, PSCA, folate receptor-a, CD44v6, CD44v7/8, avb6 integrin, 8H9, NCAM, VEGF receptors, 5T4, Foetal AchR, NKG2D ligands, dual antigen, an antigen associated with a universal tag, a cancer-testes antigen, MUC1, MUC16, NY-ESO-1, MART-1, gp100, oncofetal antigen, VEGF-R2, carcinoembryonic antigen (CEA), prostate specific antigen, PSMA, Her2/neu, estrogen receptor, progesterone receptor, ephrinB2, CD123, c-Met, GD-2, O-acetylated GD2 (OGD2), CE7, Wilms Tumor 1 (WT-1), a cyclin, cyclin A2, CCL-1, hTERT, MDM2, CYP1B, WTi, livin, AFP, p53, cyclin (D1), CS-1, BCMA, BAFF-R, TACI, CD56, TIM-3, CD123, L1-cell adhesion molecule, MAGE-A1, MAGE A3, a cyclin, such as cyclin A1 (CCNA1) and/or a pathogen-specific antigen, biotinylated molecules, molecules expressed by HIV, HCV, HBV and/or other pathogens, and/or in some aspects, neoepitopes or neoantigens thereof. In some embodiments, the antigen is associated with or is a universal tag.

In some embodiments, the plurality of antigens, e.g., the first antigen, e.g., ROR1, and the second or additional antigens, are expressed on the cell, tissue, or disease or condition being targeted, such as on the cancer cell. In some aspects, the cell, tissue, disease or condition is or is associated with a cancer or a tumor. One or more of the plurality of antigens generally also is expressed on a cell which it is not desired to target with the cell therapy, such as a normal or non-diseased cell or tissue, and/or the engineered cells themselves. In such embodiments, by requiring ligation of multiple receptors to achieve a response of the cell, specificity and/or efficacy is/are achieved.

In some embodiments, the cells and methods include multi-targeting strategies, such as expression of two or more genetically engineered receptors on the cell, each recognizing a different antigen and typically each including a different intracellular signaling component. Such multi-targeting strategies are described, for example, in WO 2014055668 (describing combinations of activating and costimulatory CARs, e.g., targeting two different antigens present individually on off-target, e.g., normal cells, but present together only on cells of the disease or condition to be treated) and Fedorov et al., Sci. Transl. Medicine, 5(215) (December, 2013) (describing cells expressing an activating and an inhibitory CAR, such as those in which the activating CAR binds to one antigen expressed on both normal or non-diseased cells and cells of the disease or condition to be treated, and the inhibitory CAR binds to another antigen expressed only on the normal cells or cells which it is not desired to treat).

In some embodiments, a plurality of cells, each engineered to express one or more recombinant receptors, are provided. For example, in some embodiments, one cell is engineered to express a binding molecule that binds and/or targets ROR1, and another cell is engineered to express a binding molecule that binds and/or targets an additional or second antigen. In some embodiments, the cells can each express a multispecific binding molecule, e.g., a multispecific recombinant receptor, where one or more of the target antigen is ROR1. In some of such embodiments, the plurality of cells can be administered together or separately. In some embodiments, some of the plurality of cells are administered simultaneously or concurrently with other cells, e.g., administered on the same day, and/or sequentially with or intermittently with, in any order, another engineered cell in the plurality. For example, in some embodiments, an engineered cell expressing a ROR1-binding molecule, e.g., CAR, is administered simultaneously with or sequentially with, in any order, another engineered cell expressing a binding molecule that binds a different target antigen or a different epitope on ROR1. In some embodiments, the plurality of cells can be in the same composition or in different compositions. Exemplary compositions of the cells include compositions described in Section III below.

### III. PHARMACEUTICAL COMPOSITIONS

Also provided are compositions including the ROR1-binding molecules, immunoconjugates, recombinant receptors, and engineered cells, including pharmaceutical compositions and formulations. Also provided are compositions comprising engineered cells that express the ROR1-binding molecules provided herein, such as recombinant receptors (e.g., CARs), including pharmaceutical compositions and formulations.

Provided are pharmaceutical formulations comprising a ROR1-binding molecule (e.g., antibody), an immunoconjugate, a recombinant receptor (e.g., chimeric antigen receptor), engineered cells expressing said molecules (e.g., antibody or recombinant receptor), a plurality of engineered cells expressing said molecules (e.g., recombinant receptor) and/or additional agents for combination treatment or therapy. The pharmaceutical compositions and formulations generally include one or more optional pharmaceutically acceptable carrier or excipient. In some embodiments, the composition includes at least one additional therapeutic agent.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

In some aspects, the choice of carrier is determined in part by the particular cell, binding molecule, and/or antibody, and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

Formulations of the antibodies described herein can include lyophilized formulations and aqueous solutions.

The formulation or composition may also contain more than one active ingredient useful for the particular indication, disease, or condition being treated with the binding molecules or cells, preferably those with activities complementary to the binding molecule or cell, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc. In some embodiments, the cells or antibodies are administered in the form of a salt, e.g., a pharmaceutically acceptable salt. Suitable pharmaceutically acceptable acid addition salts include those derived from mineral acids, such as hydrochloric, hydrobromic, phosphoric, metaphosphoric, nitric, and sulphuric acids, and organic acids, such as tartaric, acetic, citric, malic, lactic, fumaric, benzoic, glycolic, gluconic, succinic, and arylsulphonic acids, for example, p-toluenesulphonic acid.

Active ingredients may be entrapped in microcapsules, in colloidal drug delivery systems (for example, liposomes, albumin microspheres, microemulsions, nano-particles and nanocapsules) or in macroemulsions. In certain embodiments, the pharmaceutical composition is formulated as an inclusion complex, such as cyclodextrin inclusion complex, or as a liposome. Liposomes can serve to target the host cells (e.g., T-cells or NK cells) to a particular tissue. Many methods are available for preparing liposomes, such as those described in, for example, Szoka et al., Ann. Rev. Biophys. Bioeng., 9: 467 (1980), and U.S. Pat. Nos. 4,235,871, 4,501,728, 4,837,028, and 5,019,369.

The pharmaceutical composition in some aspects can employ time-released, delayed release, and sustained release delivery systems such that the delivery of the composition occurs prior to, and with sufficient time to cause, sensitization of the site to be treated. Many types of release delivery systems are available and known. Such systems can avoid repeated administrations of the composition, thereby increasing convenience to the subject and the physician.

The pharmaceutical composition in some embodiments contains the binding molecules and/or cells in amounts effective to treat or prevent the disease or condition, such as a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

In certain embodiments, in the context of genetically engineered cells containing the binding molecules, a subject is administered the range of at or about one million to at or about 100 billion cells, such as, e.g., at or about 1 million to at or about 50 billion cells (e.g., at or about 5 million cells, at or about 25 million cells, at or about 500 million cells, at or about 1 billion cells, at or about 5 billion cells, at or about 20 billion cells, at or about 30 billion cells, at or about 40 billion cells, or a range defined by any two of the foregoing values), such as at or about 10 million to at or about 100 billion cells (e.g., at or about 20 million cells, at or about 30 million cells, at or about 40 million cells, at or about 60 million cells, at or about 70 million cells, at or about 80 million cells, at or about 90 million cells, at or about 10 billion cells, at or about 25 billion cells, at or about 50 billion cells, at or about 75 billion cells, at or about 90 billion cells, or a range defined by any two of the foregoing values), and in some cases at or about 100 million cells to at or about 50 billion cells (e.g., at or about 120 million cells, at or about 250 million cells, at or about 350 million cells, at or about 450 million cells, at or about 650 million cells, at or about 800 million cells, at or about 900 million cells, at or about 3 billion cells, at or about 30 billion cells, at or about 45 billion cells) or any value in between these ranges, and/or such a number of cells per kilogram of body weight of the subject.

The may be administered using standard administration techniques, formulations, and/or devices. Provided are formulations and devices, such as syringes and vials, for storage and administration of the compositions. Administration of the cells can be autologous or heterologous. For example, immune cells, immunoresponsive cells or progenitors can be obtained from one subject, and administered to the same subject or a different, compatible subject. Peripheral blood derived immunoresponsive cells or their progeny (e.g., in vivo, ex vivo or in vitro derived) can be administered via localized injection, including catheter administration, systemic injection, localized injection, intravenous injection, or parenteral administration. When administering a therapeutic composition (e.g., a pharmaceutical composition containing a genetically modified immune cell such as a T cell), it will generally be formulated in a unit dosage injectable form (solution, suspension, emulsion).

Formulations include those for oral, intravenous, intraperitoneal, subcutaneous, pulmonary, transdermal, intramuscular, intranasal, buccal, sublingual, or suppository administration. In some embodiments, the cell populations are administered parenterally. The term “parenteral,” as used herein, includes intravenous, intramuscular, subcutaneous, rectal, vaginal, intracranial, intrathoracic, and intraperitoneal administration. In some embodiments, the cell populations are administered to a subject using peripheral systemic delivery by intravenous, intraperitoneal, or subcutaneous injection.

Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the binding molecule in a solvent, such as in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts may in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin.

Sustained-release preparations may be prepared. Suitable examples of sustained-release preparations include semipermeable matrices of solid hydrophobic polymers containing the antibody, which matrices are in the form of shaped articles, e.g. films, or microcapsules.

The formulations to be used for in vivo administration are generally sterile. Sterility may be readily accomplished, e.g., by filtration through sterile filtration membranes.

Also provided are pharmaceutical compositions for combination therapy. Any of the additional agents for combination therapy described herein, such as agents described in Section IV.B, can be prepared and administered as one or more pharmaceutical compositions, with the ROR1-binding molecule (e.g., antibody), immunoconjugate, recombinant receptor (e.g., chimeric antigen receptor) and/or engineered cells expressing said molecules (e.g., recombinant receptor) described herein. The combination therapy can be administered in one or more pharmaceutical compositions, e.g., where the binding molecules, recombinant receptors and/or cells are in the same pharmaceutical composition as the additional agent, or in separate pharmaceutical compositions. For example, in some embodiments, the additional agent is an additional engineered cell, e.g., cell engineered to express a different recombinant receptor that targets a different antigen or a different epitope on ROR1, and is administered in the same composition or in a separate composition. In some embodiments, each of the pharmaceutical composition is formulated in a suitable formulation according to the particular binding molecule, recombinant receptor, cell, e.g., engineered cell, and/or additional agent, and the particular dosage regimen and/or method of delivery.

### IV. METHODS AND USES

Also provided methods, such as methods of treatment, of using and uses of the ROR1-binding molecules, immunoconjugates, recombinant receptors, engineered cells, and pharmaceutical compositions and formulations thereof, such as in the treatment of diseases, conditions, and disorders in which ROR1 is expressed, and/or detection, diagnostic, and prognostic methods. Also provided are methods of combination therapy and/or treatment.

### A. Therapeutic and Prophylactic Methods and Uses

Also provided are methods of administering and uses, such as therapeutic and prophylactic uses, of the ROR1-binding molecules, including the anti-ROR1 antibodies, e.g., antibody fragments and proteins containing the same such as the recombinant receptors (e.g., CARs), engineered cells expressing the recombinant receptors (e.g., CARs), plurality of engineered cells expressing the receptors, and/or compositions comprising the same. Such methods and uses include therapeutic methods and uses, for example, involving administration of the molecules (e.g., ROR1-binding molecules, recombinant receptors), cells (e.g., engineered cells), or compositions containing the same, to a subject having a disease, condition, or disorder associated with ROR1 such as a disease, condition, or disorder associated with ROR1 expression, and/or in which cells or tissues express, e.g., specifically express ROR1. In some embodiments, the molecule, cell, and/or composition is administered in an effective amount to effect treatment of the disease or disorder. Provided herein are uses of the binding molecules (e.g., anti-ROR1 antibodies or antigen-binding fragments thereof), recombinant receptors (e.g., CARs), and cells (e.g., engineered cells) in such methods and treatments, and in the preparation of a medicament in order to carry out such therapeutic methods. Uses include uses of the binding molecules, CARs, antibodies, and cells in such methods and treatments, and in the preparation of a medicament in order to carry out such therapeutic methods. In some embodiments, the methods are carried out by administering the binding molecules or cells, or compositions comprising the same, to the subject having, having had, or suspected of having the disease or condition. In some embodiments, the methods thereby treat the disease or condition or disorder in the subject. Also provided herein are of use of any of the compositions, such as pharmaceutical compositions provided herein, for the treatment of a disease or disorder associated with ROR1, for example, a ROR1-expressing cancer.

As used herein, “treatment” (and grammatical variations thereof such as “treat” or “treating”) refers to complete or partial amelioration or reduction of a disease or condition or disorder, or a symptom, adverse effect or outcome, or phenotype associated therewith. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. The terms do not imply complete curing of a disease or complete elimination of any symptom or effect(s) on all symptoms or outcomes.

As used herein, “delaying development of a disease” means to defer, hinder, slow, retard, stabilize, suppress and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

“Preventing,” as used herein, includes providing prophylaxis with respect to the occurrence or recurrence of a disease in a subject that may be predisposed to the disease but has not yet been diagnosed with the disease. In some embodiments, the provided molecules and compositions are used to delay development of a disease or to slow the progression of a disease.

As used herein, to “suppress” a function or activity is to reduce the function or activity when compared to otherwise same conditions except for a condition or parameter of interest, or alternatively, as compared to another condition. For example, an antibody or composition or cell which suppresses tumor growth reduces the rate of growth of the tumor compared to the rate of growth of the tumor in the absence of the antibody or composition or cell.

An “effective amount” of an agent, e.g., a pharmaceutical formulation, binding molecule, antibody, or cells, or composition, in the context of administration, refers to an amount effective, at dosages/amounts and for periods of time necessary, to achieve a desired result, such as a therapeutic or prophylactic result.

A “therapeutically effective amount” of an agent, e.g., a pharmaceutical formulation, antibody, or cells, refers to an amount effective, at dosages and for periods of time necessary, to achieve a desired therapeutic result, such as for treatment of a disease, condition, or disorder, and/or pharmacokinetic or pharmacodynamic effect of the treatment. The therapeutically effective amount may vary according to factors such as the disease state, age, sex, and weight of the subject, and the populations of cells administered. In some embodiments, the provided methods involve administering the molecules, cells, and/or compositions at effective amounts, e.g., therapeutically effective amounts.

A “prophylactically effective amount” refers to an amount effective, at dosages and for periods of time necessary, to achieve the desired prophylactic result. Typically but not necessarily, since a prophylactic dose is used in subjects prior to or at an earlier stage of disease, the prophylactically effective amount will be less than the therapeutically effective amount.

As used herein, a “subject” is a mammal, such as a human or other animal, and typically is human.

Among the diseases to be treated are any ROR1-associated disease or condition or disease or condition in which ROR1 is specifically expressed. In certain diseases and conditions, ROR1 is expressed on malignant cells and cancers. In some embodiments, the disease or condition is a ROR1-expressing cancer. Among the ROR1-associated diseases or conditions that can be treated include, but are not limited to, B cell leukemia, lymphoma, B cell chronic lymphocytic leukemia (CLL), acute myeloid leukemia (AML), acute lymphocytic leukemia (ALL), Burkitt's Lymphoma, mantle cell lymphoma (MCL), non-small cell lung cancer (NSCLC), neuroblastoma, renal cell carcinoma, colon cancer, colorectal cancer, breast cancer, epithelial squamous cell cancer, melanoma, myeloma, stomach cancer, brain cancer, lung cancer, pancreatic cancer, cervical cancer, ovarian cancer, liver cancer, bladder cancer, prostate cancer, testicular cancer, thyroid cancer, uterine cancer, adrenal cancer and head and neck cancer. In some embodiments, the disease or condition to be treated is selected from a CLL, a MCL, an ovarian cancer, a lung cancer, a pancreatic cancer or a breast cancer. In some embodiments, the disease or condition to be treated is a CLL. In some embodiments, the disease or condition to be treated is a MCL. In some embodiments, the disease or condition to be treated is an ovarian cancer. In some embodiments, the disease or condition to be treated is a lung cancer. In some embodiments, the disease or condition to be treated is a pancreatic cancer. In some embodiments, the disease or condition to be treated is a breast cancer. In some embodiments, the disease or condition to be treated is a lung cancer, and the lung cancer is selected from among lung adenocarcinoma, adenocarcinoma, squamous cell carcinoma, small cell carcinoma, non-small cell lung cancer (NSCLC) and atypical carcinoid. In some embodiments, the disease or condition to be treated is a lung cancer, and the lung cancer is anon-small cell lung cancer (NSCLC). In some embodiments, the disease or condition to be treated is a breast cancer, and the breast cancer is a triple-negative breast cancer.

In some aspects, in certain diseases and conditions, ROR1 is expressed on malignant cells and cancers, including cancers associated with a solid tumor or a hematologic malignancy. In some embodiments, the disease or condition is a ROR1-expressing cancer. In some embodiments among the disease or disorder to be treated is a cancer, such as a ROR1-expressing cancer. In some embodiments, the cancer is associated with a solid tumor or a hematologic malignancy.

In some embodiments, the cancer is associated with a ROR1-expressing solid tumor. In some embodiments, the disease or disorder to be treated is a cancer associated with a solid tumor, such as neuroblastoma, renal cell carcinoma, colon cancer, colorectal cancer, breast cancer, epithelial squamous cell cancer, melanoma, myeloma, stomach cancer, brain cancer, lung cancer, pancreatic cancer, cervical cancer, ovarian cancer, liver cancer, bladder cancer, prostate cancer, testicular cancer, thyroid cancer, uterine cancer, adrenal cancer and head and neck cancer. In some embodiments, the lung cancer is a non-small cell lung cancer (NSCLC). In some embodiments, the breast cancer is a triple negative breast cancer (TNBC). In some aspects, triple negative breast cancer refers to a breast cancer that is negative for expression of estrogen receptors, progesterone receptors and human epidermal growth factor receptor 2 (HER2) overexpression.

In some embodiments, the cancer is associated with a ROR1-expressing hematologic malignancy. In some embodiments, the disease or disorder associated with ROR1 is a B cell-related disorder. In some embodiments, the disease or disorder to be treated is a cancer associated with a hematologic malignancy, such as B cell leukemia, lymphoma, B cell chronic lymphocytic leukemia (CLL), acute myeloid leukemia (AML), acute lymphocytic leukemia (ALL), Burkitt's Lymphoma or mantle cell lymphoma (MCL).

In some embodiments, the methods include administering engineered cells (e.g. T cells) expressing a provided binding molecule to a subject having a ROR1-associated disease or condition, such as a ROR1-expressing cancer. In particular embodiments, the engineered cells (e.g. T cells) express a recombinant receptor, such as a CAR, containing as an antigen-binding domain a provided antibody or antigen-binding fragment thereof (e.g. scFv). In some embodiments, the engineered cells are autologous to the subject being treated. In some embodiments, the engineered cells are allogeneic to the subject being treated, in which case the cells are obtained from the diseased subject and engineered with the recombinant receptor (e.g. CAR) prior to administration of the engineered cells.

In some contexts, engineering primary T cells obtained from subjects with diseases or disorders such as chronic lymphocytic leukemia (CLL) to express recombinant receptors has been reported to result in engineered T cells with impaired function, compared to primary T cells from subjects with other hematologic malignancies (see, e.g., Gorgun et al., J Clin Invest. 2005 July; 115(7):1797-805; Ramsay et al., J Clin Invest. 2008 July; 118(7):2427-37; Riches et al., Discov Med. 2013 Dec.; 16(90):295-302). In some cases, as described herein, the provided recombinant receptors can be expressed from primary T cells obtained from subjects with CLL, without observed impairment in function. In some aspects, engineered cells expressing provided binding molecules, e.g., recombinant receptors targeting ROR1, can be successfully generated using cells from subjects with diseases or disorders such as CLL.

In some embodiments, the methods may identify a subject who has, is suspected to have, or is at risk for developing a ROR1-associated disease or disorder. Hence, provided are methods for identifying subjects with diseases or disorders associated with elevated ROR1 expression and selecting them for treatment with a provided ROR1 binding molecule, including any of the anti-ROR1 antibodies, e.g., antibody fragments and proteins containing the same such as the chimeric receptors, and/or engineered cells expressing the recombinant receptors.

For example, a subject may be screened for the presence of a disease or disorder associated with elevated ROR1 expression, such as a ROR1-expressing cancer. In some embodiments, the methods include screening for or detecting the presence of a ROR1-associated disease, e.g. a tumor. Thus, in some aspects, a sample may be obtained from a patient suspected of having a disease or disorder associated with elevated ROR1 expression and assayed for the expression level of ROR1. In some aspects, a subject who tests positive for a ROR1-associated disease or disorder may be selected for treatment by the present methods, and may be administered a therapeutically effective amount of an anti-ROR1 antibody, a ROR1-targeting CAR, cells containing a CAR or a pharmaceutical composition thereof as described herein. In some embodiments, the methods can be used to monitor the size or density of a ROR1-expressing tissue, e.g. tumor, over time, e.g., before, during, or after treatment by the methods.

In some embodiments, the subject has persistent or relapsed disease, e.g., following treatment with another ROR1-specific antibody and/or cells expressing a ROR1-targeting chimeric receptor and/or other therapy, including chemotherapy, radiation, and/or hematopoietic stem cell transplantation (HSCT), e.g., allogenic HSCT. In some embodiments, the administration effectively treats the subject despite the subject having become resistant to another ROR1-targeted therapy. In some embodiments, the subject has not relapsed but is determined to be at risk for relapse, such as at a high risk of relapse, and thus the compound or composition is administered prophylactically, e.g., to reduce the likelihood of or prevent relapse.

In some embodiments, the treatment does not induce an immune response by the subject to the therapy, and/or does not induce such a response to a degree that prevents effective treatment of the disease or condition. In some aspects, the degree of immunogenicity and/or graft versus host response is less than that observed with a different but comparable treatment. For example, in the case of adoptive cell therapy using cells expressing CARs including the provided anti-ROR1 antibodies, the degree of immunogenicity in some embodiments is reduced compared to CARs including a different antibody that binds to a similar, e.g., overlapping epitope and/or that competes for binding to ROR1 with the provided antibody, such as a mouse or rabbit or humanized antibody.

In some embodiments, the methods include adoptive cell therapy, whereby genetically engineered cells expressing the provided ROR1-targeting receptors (e.g., containing ROR1-targeting antibody or fragment thereof) are administered to subjects. Such administration can promote activation of the cells (e.g., T cell activation) in a ROR1-targeting manner, such that the cells of the disease or disorder are targeted for destruction.

Thus, the provided methods and uses include methods and uses for adoptive cell therapy. In some embodiments, the methods include administration of the cells or a composition containing the cells to a subject, tissue, or cell, such as one having, at risk for, or suspected of having the disease, condition or disorder. In some embodiments, the cells, populations, and compositions are administered to a subject having the particular disease or condition to be treated, e.g., via adoptive cell therapy, such as adoptive T cell therapy. In some embodiments, the cells or compositions are administered to the subject, such as a subject having or at risk for the disease or condition. In some aspects, the methods thereby treat, e.g., ameliorate one or more symptom of the disease or condition, such as by lessening tumor burden in a ROR1-expressing cancer.

Methods for administration of cells for adoptive cell therapy are known and may be used in connection with the provided methods and compositions. For example, adoptive T cell therapy methods are described, e.g., in US Patent Application Publication No. 2003/0170238 to Gruenberg et al; U.S. Pat. No. 4,690,915 to Rosenberg; Rosenberg (2011) Nat Rev Clin Oncol. 8(10):577-85). See, e.g., Themeli et al. (2013) Nat Biotechnol. 31(10): 928-933; Tsukahara et al. (2013) Biochem Biophys Res Commun 438(1): 84-9; Davila et al. (2013) PLoS ONE 8(4): e61338.

In some embodiments, the cell therapy, e.g., adoptive cell therapy, e.g., adoptive T cell therapy, is carried out by autologous transfer, in which the cells are isolated and/or otherwise prepared from the subject who is to receive the cell therapy, or from a sample derived from such a subject. Thus, in some aspects, the cells are derived from a subject, e.g., patient, in need of a treatment and the cells, following isolation and processing are administered to the same subject.

In some embodiments, the cell therapy, e.g., adoptive cell therapy, e.g., adoptive T cell therapy, is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject.

In some embodiments, the subject, to whom the cells, cell populations, or compositions are administered is a primate, such as a human. In some embodiments, the primate is a monkey or an ape. The subject can be male or female and can be any suitable age, including infant, juvenile, adolescent, adult, and geriatric subjects. In some embodiments, the subject is a non-primate mammal, such as a rodent. In some examples, the patient or subject is a validated animal model for disease, adoptive cell therapy, and/or for assessing toxic outcomes such as cytokine release syndrome (CRS).

The ROR1-binding molecules, such as antibodies and chimeric receptors containing the antibodies and cells expressing the same, can be administered by any suitable means, for example, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, intracranial, intrathoracic, or subcutaneous administration. Dosing and administration may depend in part on whether the administration is brief or chronic. Various dosing schedules include but are not limited to single or multiple administrations over various time-points, bolus administration, and pulse infusion.

For the prevention or treatment of disease, the appropriate dosage of the binding molecule or cell may depend on the type of disease to be treated, the type of binding molecule, the severity and course of the disease, whether the binding molecule is administered for preventive or therapeutic purposes, previous therapy, the patient's clinical history and response to the binding molecule, and the discretion of the attending physician. The compositions and molecules and cells are in some embodiments suitably administered to the patient at one time or over a series of treatments.

Depending on the type and severity of the disease, dosages of antibodies may include at or about 1 μg/kg to at or about 15 mg/kg (e.g. at or about 0.1 mg/kg—at or about 10 mg/kg), at or about 1 μg/kg to at or about 100 mg/kg or more, at or about 0.05 mg/kg to at or about 10 mg/kg, at or about 0.5 mg/kg, at or about 2.0 mg/kg, at or about 4.0 mg/kg or at or about 10 mg/kg. Multiple doses may be administered intermittently, e.g. every week or every three weeks. An initial higher loading dose, followed by one or more lower doses may be administered.

In certain embodiments, the cells, or individual populations of sub-types of cells, are administered to the subject at a range of at or about 0.1 million to at or about 100 billion cells and/or that amount of cells per kilogram of body weight of the subject, such as, e.g., at or about 0.1 million to at or about 50 billion cells (e.g., at or about 5 million cells, at or about 25 million cells, at or about 500 million cells, at or about 1 billion cells, at or about 5 billion cells, at or about 20 billion cells, at or about 30 billion cells, at or about 40 billion cells, or a range defined by any two of the foregoing values), at or about 1 million to at or about 50 billion cells (e.g., at or about 5 million cells, at or about 25 million cells, at or about 500 million cells, at or about 1 billion cells, at or about 5 billion cells, at or about 20 billion cells, at or about 30 billion cells, at or about 40 billion cells, or a range defined by any two of the foregoing values), such as at or about 10 million to at or about 100 billion cells (e.g., at or about 20 million cells, at or about 30 million cells, at or about 40 million cells, at or about 60 million cells, at or about 70 million cells, at or about 80 million cells, at or about 90 million cells, at or about 10 billion cells, at or about 25 billion cells, at or about 50 billion cells, at or about 75 billion cells, at or about 90 billion cells, or a range defined by any two of the foregoing values), and in some cases at or about 100 million cells to at or about 50 billion cells (e.g., at or about 120 million cells, at or about 250 million cells, at or about 350 million cells, at or about 650 million cells, at or about 800 million cells, at or about 900 million cells, at or about 3 billion cells, at or about 30 billion cells, at or about 45 billion cells) or any value in between these ranges and/or per kilogram of body weight of the subject. Dosages may vary depending on attributes particular to the disease or disorder and/or patient and/or other treatments. In some embodiments, such values refer to numbers of recombinant receptor-expressing cells; in other embodiments, they refer to number of T cells or PBMCs or total cells administered.

In some embodiments, for example, where the subject is a human, the dose includes fewer than about 5×10⁸ total recombinant receptor (e.g., CAR)-expressing cells, T cells, or peripheral blood mononuclear cells (PBMCs), e.g., in the range of at or about 1×10⁶ to at or about 5×10⁸ such cells, such as at or about 2×10⁶, 5×10⁶, 1×10⁷, 5×10⁷, 1×10⁸, 1.5×10⁹, or 5×10⁸ total such cells, or the range between any two of the foregoing values. In some embodiments, for example, where the subject is a human, the dose includes more than at or about 1×10⁶ total recombinant receptor (e.g., CAR)-expressing cells, T cells, or peripheral blood mononuclear cells (PBMCs) and fewer than at or about 2×10⁹ total recombinant receptor (e.g., CAR)-expressing cells, T cells, or peripheral blood mononuclear cells (PBMCs), e.g., in the range of at or about 2.5×10⁷ to at or about 1.2×10⁹ such cells, such as at or about 2.5×10⁷, 5×10⁷, 1×10⁸, 1.5×10⁹, 8×10⁹, or 1.2×10⁹ total such cells, or the range between any two of the foregoing values.

In some embodiments, the dose of genetically engineered cells comprises from at least or at least about or is 1×10⁵ to at or about 5×10⁸ total CAR-expressing (CAR*) T cells, from at or about 1×10⁵ to at or about 2.5×10⁸ total CAR+ T cells, from at or about 1×10⁵ to at or about 1×10⁸ total CAR+ T cells, from at or about 1×10⁵ to at or about 5×10⁷ total CAR+ T cells, from at or about 1×10⁵ to at or about 2.5×10⁷ total CAR⁺ T cells, from at or about 1×10⁵ to at or about 1×10⁷ total CAR⁺ T cells, from at or about 1×10⁵ to at or about 5×10⁶ total CAR+ T cells, from at or about 1×10⁵ to at or about 2.5×10⁶ total CAR⁺ T cells, from at or about 1×10⁵ to at or about 1×10⁶ total CAR⁺ T cells, from at or about 1×10⁶ to at or about 5×10⁸ total CAR⁺ T cells, from at or about 1×10⁶ to at or about 2.5×10⁸ total CAR⁺ T cells, from at or about 1×10⁶ to at or about 1×10⁸ total CAR⁺ T cells, from at or about 1×10⁶ to at or about 5×10⁷ total CAR⁺ T cells, from at or about 1×10⁶ to at or about 2.5×10⁷ total CAR⁺ T cells, from at or about 1×10⁶ to at or about 1×10⁷ total CAR⁺ T cells, from at or about 1×10⁶ to at or about 5×10⁶ total CAR⁺ T cells, from at or about 1×10⁶ to at or about 2.5×10⁶ total CAR⁺ T cells, from at or about 2.5×10⁶ to at or about 5×10⁸ total CAR⁺ T cells, from at or about 2.5×10⁶ to at or about 2.5×10⁸ total CAR⁺ T cells, from at or about 2.5×10⁶ to at or about 1×10⁸ total CAR⁺ T cells, from at or about 2.5×10⁶ to at or about 5×10⁷ total CAR⁺ T cells, from at or about 2.5×10⁶ to at or about 2.5×10⁷ total CAR⁺ T cells, from at or about 2.5×10⁶ to at or about 1×10⁷ total CAR⁺ T cells, from at or about 2.5×10⁶ to at or about 5×10⁶ total CAR+ T cells, from at or about 5×10⁶ to at or about 5×10⁸ total CAR⁺ T cells, from at or about 5×10⁶ to at or about 2.5×10⁸ total CAR⁺ T cells, from at or about 5×10⁶ to at or about 1×10⁸ total CAR⁺ T cells, from at or about 5×10⁶ to at or about 5×10⁷ total CAR+ T cells, from at or about 5×10⁶ to at or about 2.5×10⁷ total CAR+ T cells, from at or about 5×10⁶ to at or about 1×10⁷ total CAR+ T cells, from at or about 1×10⁷ to at or about 5×10⁸ total CAR⁺ T cells, from at or about 1×10⁷ to at or about 2.5×10⁸ total CAR⁺ T cells, from at or about 1×10⁷ to at or about 1×10⁸ total CAR+ T cells, from at or about 1×10⁷ to at or about 5×10⁷ total CAR+ T cells, from at or about 1×10⁷ to at or about 2.5×10⁷ total CAR+ T cells, from at or about 2.5×107 to at or about 5×10⁸ total CAR+ T cells, from at or about 2.5×10⁷ to at or about 2.5×10⁸ total CAR+ T cells, from at or about 2.5×10⁷ to at or about 1×10⁸ total CAR+ T cells, from at or about 2.5×107 to at or about 5×10⁷ total CAR+ T cells, from at or about 5×10⁷ to at or about 5×10⁸ total CAR+ T cells, from at or about 5×10⁷ to at or about 2.5×10⁸ total CAR+ T cells, from at or about 5×10⁷ to at or about 1×10⁸ total CAR+ T cells, from at or about 1×10⁸ to at or about 5×10⁸ total CAR+ T cells, from at or about 1×10⁸ to at or about 2.5×10⁸ total CAR+ T cells, from at or about or 2.5×10⁸ to at or about 5×10⁸ total CAR+ T cells. In some embodiments, the dose of genetically engineered cells comprises from or from about 2.5×10⁷ to at or about 1.5×10⁸ total CAR+ T cells, such as from or from about 5×10⁷ to or to about 1×10⁸ total CAR+ T cells.

In some embodiments, the dose of genetically engineered cells comprises at least at or about 1×10⁵ CAR⁺ cells, at least at or about 2.5×10⁵ CAR⁺ cells, at least at or about 5×10⁵ CAR⁺ cells, at least at or about 1×10⁶ CAR⁺ cells, at least at or about 2.5×10⁶ CAR⁺ cells, at least at or about 5×10⁶ CAR⁺ cells, at least at or about 1×10⁷ CAR⁺ cells, at least at or about 2.5×10⁷ CAR⁺ cells, at least at or about 5×10⁷ CAR⁺ cells, at least at or about 1×10⁸ CAR⁺ cells, at least at or about 1.5×10⁸ CAR⁺ cells, at least at or about 2.5×10⁸ CAR⁺ cells, or at least at or about 5×10⁸ CAR⁺ cells.

In some embodiments, the cell therapy comprises administration of a dose comprising a number of cell from or from about 1×10⁵ to or to about 5×10⁸ total recombinant receptor-expressing cells, total T cells, or total peripheral blood mononuclear cells (PBMCs), from or from about 5×10⁵ to or to about 1×10⁷ total recombinant receptor-expressing cells, total T cells, or total peripheral blood mononuclear cells (PBMCs) or from or from about 1×10⁶ to or to about 1×10⁷ total recombinant receptor-expressing cells, total T cells, or total peripheral blood mononuclear cells (PBMCs), each inclusive. In some embodiments, the cell therapy comprises administration of a dose of cells comprising a number of cells at least or at least about 1×10⁵ total recombinant receptor-expressing cells, total T cells, or total peripheral blood mononuclear cells (PBMCs), such at least or at least 1×10⁶, at least or at least about 1×10⁷, at least or at least about 1×10⁸ of such cells. In some embodiments, the number is with reference to the total number of CD3+ or CD8⁺, in some cases also recombinant receptor-expressing (e.g. CAR⁺) cells. In some embodiments, the cell therapy comprises administration of a dose comprising a number of cell from or from about 1×10⁵ to or to about 5×10⁸ CD3⁺ or CD8⁺ total T cells or CD3⁺ or CD8⁺ recombinant receptor-expressing cells, from or from about 5×10⁵ to or to about 1×10⁷ CD3⁺ or CD8⁺ total T cells or CD3⁺ or CD8⁺ recombinant receptor-expressing cells, or from or from about 1×10⁶ to or to about 1×10⁷ CD3⁺ or CD8⁺ total T cells or CD3⁺ or CD8⁺ recombinant receptor-expressing cells, each inclusive. In some embodiments, the cell therapy comprises administration of a dose comprising a number of cell from or from about 1×10⁵ to or to about 5×10⁸ total CD3⁺/CAR⁺ or CD8⁺/CAR⁺ cells, from or from about 5×10⁵ to or to about 1×10⁷ total CD3⁺/CAR⁺ or CD8+/CAR⁺ cells, or from or from about 1×10⁶ to or to about 1×10⁷ total CD3⁺/CAR⁺ or CD8+/CAR⁺ cells, each inclusive.

In some embodiments, the T cells of the dose include CD4+ T cells, CD8+ T cells or CD4+ and CD8+ T cells.

In some embodiments, for example, where the subject is human, the CD8+ T cells of the dose, including in a dose including CD4⁺ and CD8⁺ T cells, includes between at or about 1×10⁶ and at or about 5×10⁸ total recombinant receptor (e.g., CAR)-expressing CD8⁺ cells, e.g., in the range of from at or about 5×10⁶ to at or about 1×10⁸ such cells, such as 1×10⁷, 2.5×10⁷, 5×10⁷, 7.5×10⁷, 1×10⁸, 1.5×10⁸, or 5×10⁸ total such cells, or the range between any two of the foregoing values. In some embodiments, the patient is administered multiple doses, and each of the doses or the total dose can be within any of the foregoing values. In some embodiments, the dose of cells comprises the administration of from or from about 1×10⁷ to or to about 0.75×10⁸ total recombinant receptor-expressing CD8⁺ T cells, from or from about 1×10⁷ to or to about 5×10⁷ total recombinant receptor-expressing CD8+ T cells, from or from about 1×10⁷ to or to about 0.25×10⁸ total recombinant receptor-expressing CD8⁺ T cells, each inclusive. In some embodiments, the dose of cells comprises the administration of at or about 1×10⁷, 2.5×10⁷, 5×10⁷, 7.5×10⁷, 1×10⁸, 1.5×10⁸, 2.5×10⁸, or 5×10⁸ total recombinant receptor-expressing CD8⁺ T cells.

In some embodiments, the dose of cells, e.g., recombinant receptor-expressing T cells, is administered to the subject as a single dose or is administered only one time within a period of two weeks, one month, three months, six months, 1 year or more.

In some embodiments, the cells or antibodies are administered as part of a combination treatment, such as simultaneously with or sequentially with, in any order, another therapeutic intervention, such as another antibody or engineered cell or receptor or agent, such as a cytotoxic or therapeutic agent.

The cells or antibodies in some embodiments are co-administered with one or more additional therapeutic agents or in connection with another therapeutic intervention, either simultaneously or sequentially in any order. In some contexts, the cells are co-administered with another therapy sufficiently close in time such that the cell populations enhance the effect of one or more additional therapeutic agents, or vice versa. In some embodiments, the cells or antibodies are administered prior to the one or more additional therapeutic agents. In some embodiments, the cells or antibodies are administered after to the one or more additional therapeutic agents.

Once the cells are administered to a mammal (e.g., a human), the biological activity of the engineered cell populations and/or antibodies in some aspects is measured by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells can be measured using any suitable known methods, such as cytotoxicity assays described in, for example, Kochenderfer et al., J. Immunotherapy, 32(7): 689-702 (2009), and Herman et al. J. Immunological Methods, 285(1): 25-40 (2004). In certain embodiments, the biological activity of the cells also can be measured by assaying expression and/or secretion of certain cytokines, such as CD 107a, IFNγ, IL-2, and TNF. In some aspects the biological activity is measured by assessing clinical outcome, such as reduction in tumor burden or load.

In certain embodiments, engineered cells are modified in any number of ways, such that their therapeutic or prophylactic efficacy is increased. For example, the engineered CAR or TCR expressed by the population in some embodiments are conjugated either directly or indirectly through a linker to a targeting moiety. The practice of conjugating compounds, e.g., the CAR or TCR, to targeting moieties is known. See, for instance, Wadwa et al., J. Drug Targeting 3: 1 1 1 (1995), and U.S. Pat. No. 5,087,616.

### B. Combination Therapy

Also provided are methods of combination therapy that includes administering and uses, such as therapeutic and prophylactic uses, of the ROR1-binding molecules, including the anti-ROR1 antibodies, e.g., antibody fragments, conjugates, and proteins containing the same, such as the recombinant receptors (e.g., CARs), engineered cells expressing the recombinant receptors (e.g., CARs), plurality of engineered cells expressing the receptors, and/or compositions comprising the same.

In some embodiments, the ROR1-binding molecule (e.g., antibody), immunoconjugate, recombinant receptor (e.g., chimeric antigen receptor) and/or engineered cells expressing said molecules (e.g., recombinant receptor) described herein are administered as part of a combination treatment or combination therapy, such as simultaneously with, sequentially with or intermittently with, in any order, one or more additional therapeutic intervention. In some embodiments, the one or more additional therapeutic intervention includes, for example, an antibody, an engineered cell, a receptor and/or an agent, such as a cell expressing a recombinant receptor, and/or cytotoxic or therapeutic agent, e.g., a chemotherapeutic agent. In some embodiments, the combination therapy includes administration of one or more additional agents, therapies and/or treatments, e.g., any of the additional agents, therapy and/or treatments described herein. In some embodiments, the combination therapy includes administration of one or more additional agents for treatment or therapy, such as an immunomodulatory agent, immune checkpoint inhibitor, adenosine pathway or adenosine receptor antagonist or agonist and kinase inhibitors. In some embodiments, the combination treatment or combination therapy includes an additional treatment, such as a surgical treatment, transplant, and/or radiation therapy. Also provided are methods of combination treatment or combination therapy that includes administering the binding molecules (e.g., ROR1-binding molecules), recombinant receptors, cells and/or compositions described herein and one or more additional therapeutic interventions.

In some embodiments, the additional agent for combination treatment or combination therapy enhances, boosts and/or promotes the efficacy and/or safety of the therapeutic effect of binding molecules, recombinant receptors, cells and/or compositions. In some embodiments, the additional agent enhances or improves the efficacy, survival or persistence of the administered cells, e.g., cells expressing the binding molecule or a recombinant receptor. In some embodiments, the additional agent is selected from among a protein phosphatase inhibitor, a kinase inhibitor, a cytokine, an immunomodulator, or an agent that decreases the level or activity of a regulatory T (Treg) cell. In some embodiments, the additional agent enhances safety, by virtue of reducing or ameliorating adverse effects of the administered binding molecules, recombinant receptors, cells and/or compositions. In some embodiments, the additional agent can treat the same disease, condition or a comorbidity. In some embodiments, the additional agent can ameliorate, reduce or eliminate one or more toxicities, adverse effects or side effects that are associated with administration of the binding molecules, recombinant receptors, cells and/or compositions, e.g., CAR-expressing cells.

In some embodiments, the additional therapy, treatment or agent includes chemotherapy, radiation therapy, surgery, transplantation, adoptive cell therapy, antibodies, cytotoxic agents, chemotherapeutic agents, cytokines, growth inhibitory agents, anti-hormonal agents, kinase inhibitors, anti-angiogenic agents, cardioprotectants, immunostimulatory agents, immunosuppressive agents, immune checkpoint inhibitors, antibiotics, angiogenesis inhibitors, metabolic modulators or other therapeutic agents or any combination thereof. In some embodiments, the additional agent is a protein, a peptide, a nucleic acid, a small molecule agent, a cell, a toxin, a lipid, a carbohydrate or combinations thereof, or any other type of therapeutic agent, e.g. radiation. In some embodiments, the additional therapy, agent or treatment includes surgery, chemotherapy, radiation therapy, transplantation, administration of cells expressing a recombinant receptor, e.g., CAR, kinase inhibitor, immune checkpoint inhibitor, mTOR pathway inhibitor, immunosuppressive agents, immunomodulators, antibodies, immunoablative agents, antibodies and/or antigen binding fragments thereof, antibody conjugates, other antibody therapies, cytotoxins, steroids, cytokines, peptide vaccines, hormone therapy, antimetabolites, metabolic modulators, drugs that inhibit either the calcium dependent phosphatase calcineurin or the p70S6 kinase FK506) or inhibit the p70S6 kinase, alkylating agents, anthracyclines, vinca alkaloids, proteasome inhibitors, GITR agonists, protein tyrosine phosphatase inhibitors, protein kinase inhibitors, an oncolytic virus, and/or other types of immunotherapy. In some embodiments, the additional agent or treatment is bone marrow transplantation, T cell ablative therapy using chemotherapy agents such as, fludarabine, external-beam radiation therapy (XRT), cyclophosphamide, and/or antibody therapy.

In some embodiments, the cells, binding molecules (e.g., ROR1-binding molecules), recombinant receptors and/or compositions, e.g., CAR-expressing cells, are administered in combination with other engineered cells, e.g., other CAR-expressing cells. In some embodiments, the additional agent is a kinase inhibitor, e.g., an inhibitor of Bruton's tyrosine kinase (Btk), e.g., ibrutinib. In some embodiments, the additional agent is an adenosine pathway or adenosine receptor antagonist or agonist. In some embodiments, the additional agent is an immunomodulator such as thalidomide or a thalidomide derivative (e.g., lenalidomide). In some embodiments, the additional therapy, agent or treatment is a cytotoxic or chemotherapy agent, a biologic therapy (e.g., antibody, e.g., monoclonal antibody, or cellular therapy), or an inhibitor (e.g., kinase inhibitor).

In some embodiments, a chemotherapeutic agent (sometimes referred to as a cytotoxic agent) is administered to the subject to disrupt a lesion. In certain embodiments, the lesion is tumor. In particular embodiments, the lesion is cancerous. In particular embodiments, the chemotherapeutic agent is any agent known to those of skill in the art to be effective for the treatment, prevention or amelioration of hyperproliferative disorders such as cancer. Chemotherapeutic agents include, but are not limited to, small molecules, synthetic drugs, peptides, polypeptides, proteins, nucleic acids (e.g., DNA and RNA polynucleotides including, but not limited to, antisense nucleotide sequences, triple helices and nucleotide sequences encoding biologically active proteins, polypeptides or peptides), antibodies, synthetic or natural inorganic molecules, mimetic agents, and synthetic or natural organic molecules. In particular embodiments, chemotherapeutic drugs include alkylating agents, anthracyclines, cytoskeletal disruptors (taxanes), epothilones, histone deacetylase inhibitors, topoisomerase inhibitors, topoisomerase II inhibitors, kinase inhibitors, nucleotide analogs and precursor analogs, peptide antibiotics, platinum-based agents, and vinca alkaloids and derivatives.

In certain embodiments, a lesion is disrupted by administering a chemotherapeutic agent to modulate genetically engineered cells in vivo. Chemotherapeutic agents may include, but are not limited to, abarelix, aldesleukin, alemtuzumab, alitretinoin, allopurinol, altretamine, amifostine, anastrozole, arsenic trioxide, asparaginase, BCG live, bevaceizumab, bexarotene, bleomycin, bortezomib, busulfan, calusterone, camptothecin, capecitabine, carboplatin, carmustine, celecoxib, cetuximab, chlorambucil, cinacalcet, cisplatin, cladribine, cyclophosphamide, cytarabine, dacarbazine, dactinomycin, darbepoetin alfa, daunorubicin, denileukin diftitox, dexrazoxane, docetaxel, doxorubicin, dromostanolone, Elliott's B solution, epirubicin, epoetin alfa, estramustine, etoposide, exemestane, filgrastim, floxuridine, fludarabine, fluorouracil, fulvestrant, gemcitabine, gemtuzumab ozogamicin, gefitinib, goserelin, hydroxyurea, ibritumomab tiuxetan, idarubicin, ifosfamide, imatinib, interferon alfa-2a, interferon alfa-2b, irinotecan, letrozole, leucovorin, levamisole, lomustine, meclorethamine, megestrol, melphalan, mercaptopurine, mesna, methotrexate, methoxsalen, methylprednisolone, mitomycin C, mitotane, mitoxantrone, nandrolone, nofetumomab, oblimersen, oprelvekin, oxaliplatin, paclitaxel, pamidronate, pegademase, pegaspargase, pegfilgrastim, pemetrexed, pentostatin, pipobroman, plicamycin, polifeprosan, porfimer, procarbazine, quinacrine, rasburicase, rituximab, sargramostim, streptozocin, talc, tamoxifen, tarceva, temozolomide, teniposide, testolactone, thioguanine, thiotepa, topotecan, toremifene, tositumomab, trastuzumab, tretinoin, uracil mustard, valrubicin, vinblastine, vincristine, vinorelbine, and zoledronate.

In some embodiments, exemplary chemotherapeutic agents include an anthracycline (e.g., doxorubicin, such as liposomal doxorubicin); a vinca alkaloid (e.g., vinblastine, vincristine, vindesine, vinorelbine); an alkylating agent (e.g., cyclophosphamide, decarbazine, melphalan, ifosfamide, temozolomide); an immune cell antibody (e.g., alemtuzumab, gemtuzumab, rituximab, tositumomab); an antimetabolite (including, e.g., folic acid antagonists, pyrimidine analogs, purine analogs and adenosine deaminase inhibitors such as fludarabine); a TNFR glucocorticoid induced TNFR related protein (GITR) agonist; a proteasome inhibitor (e.g., aclacinomycin A, gliotoxin or bortezomib); an immunomodulatory such as thalidomide or a thalidomide derivative (e.g., lenalidomide).

In some embodiments, the additional therapy or treatment is cell therapy, e.g., adoptive cell therapy. In some embodiments, the additional therapy includes administration of engineered cells, e.g., additional CAR-expressing cell. In some embodiments, the additional engineered cell is a CAR-expressing cell that expresses the same or different recombinant receptor as the engineered cells provided herein, e.g., anti-ROR1 CAR-expressing cells. In some embodiments, the recombinant receptor, e.g., CAR, expressed on the additional engineered cell, recognizes a different antigen and/or epitope. In some embodiments, the recombinant receptor, e.g., CAR, expressed on the additional engineered cell, recognizes a different epitope of the same antigen as the recombinant receptors described herein, e.g., ROR1. In some embodiments, the recombinant receptor, e.g., CAR, expressed on the additional engineered cell, recognizes a different antigen, e.g., a different tumor antigen or combination of antigens. For example, in some embodiments, the recombinant receptor, e.g., CAR, expressed on the additional engineered cell, targets cancer cells that express early lineage markers, e.g., cancer stem cells, while other CAR-expressing cells target cancer cells that express later lineage markers. In such embodiments, the additional engineered cell is administered prior to, concurrently with, or after administration (e.g., infusion) of the CAR-expressing cells described herein. In some embodiments, the additional engineered cell expresses allogeneic CAR.

In some embodiments, the configurations of one or more of the CAR molecules comprise a primary intracellular signaling domain and two or more, e.g., 2, 3, 4, or 5 or more, costimulatory signaling domains. In some embodiments, the one or more of the CAR molecules may have the same or a different primary intracellular signaling domain, the same or different costimulatory signaling domains, or the same number or a different number of costimulatory signaling domains. In some embodiments, the one or more of the CAR molecules can be configured as a split CAR, in which one of the CAR molecules comprises an antigen binding domain and a costimulatory domain (e.g., 4-1BB), while the other CAR molecule comprises an antigen binding domain and a primary intracellular signaling domain (e.g., CD3 zeta).

In some embodiments, the additional agent is any of the multispecific binding molecules and/or cells engineered to express one or more of the binding molecules described herein and/or cells engineered to express additional binding molecules, e.g., recombinant receptors, e.g., CAR, that target a different antigen. In some embodiments, the additional agent includes any of the cells or plurality of cells described herein, e.g., in Section I.C. In some embodiments, the additional agent is a cell engineered to express a recombinant receptor, e.g., CAR, targeting a different epitope and/or antigen, e.g., a different antigen associated with a disease or condition. In some embodiments, the additional agent is a cell engineered to express a recombinant receptor, e.g., CAR, targeting a second or additional antigen expressed in the disease or disorder, e.g., a cancer or a tumor.

In some embodiments, the additional agent is an immunomodulatory agent. In some embodiments, the combination therapy includes an immunomodulatory agent that can stimulate, amplify and/or otherwise enhance an anti-tumor immune response, e.g., anti-tumor immune response from the administered engineered cells, such as by inhibiting immunosuppressive signaling or enhancing immunostimulant signaling. In some embodiments, the immunomodulatory agent is a peptide, protein or is a small molecule. In some embodiments, the protein can be a fusion protein or a recombinant protein.

In some embodiments, the immunomodulatory agent binds to an immunologic target, such as a cell surface receptor expressed on immune cells, such a T cells, B cells or antigen-presenting cells. For example, in some embodiments, the immunomodulatory agent is an antibody or antigen-binding antibody fragment, a fusion protein, a small molecule or a polypeptide. In some embodiments, the binding molecules, recombinant receptors, cells and/or compositions are administered in combination with an additional agent that is an antibody or an antigen-binding fragment thereof, such as a monoclonal antibody.

In some embodiments, the immunomodulatory agent blocks, inhibits or counteracts a component of the immune checkpoint pathway. The immune system has multiple inhibitory pathways that are involved in maintaining self-tolerance and for modulating immune responses. Tumors can use certain immune-checkpoint pathways as a major mechanism of immune resistance, particularly against T cells that are specific for tumor antigens (Pardoll (2012) Nature Reviews Cancer 12:252-264), e.g., engineered cells such as CAR-expressing cells. Because many such immune checkpoints are initiated by ligand-receptor interactions, they can be readily blocked by antibodies against the ligands and/or their receptors.

Therefore, therapy with antagonistic molecules blocking an immune checkpoint pathway, such as small molecules, nucleic acid inhibitors (e.g., RNAi) or antibody molecules, are becoming promising avenues of immunotherapy for cancer and other diseases. In contrast to the majority of anti-cancer agents, checkpoint inhibitors do not necessarily target tumor cells directly, but rather target lymphocyte receptors or their ligands in order to enhance the endogenous antitumor activity of the immune system.

As used herein, the term “immune checkpoint inhibitor” refers to molecules that totally or partially reduce, inhibit, interfere with or modulate one or more checkpoint proteins. Checkpoint proteins regulate T-cell activation or function. These proteins are responsible for co-stimulatory or inhibitory interactions of T-cell responses. Immune checkpoint proteins regulate and maintain self-tolerance and the duration and amplitude of physiological immune responses. In some embodiments, the subject can be administered an additional agent that can enhance or boost the immune response, e.g., immune response effected by the binding molecules (e.g., ROR1-binding molecules), recombinant receptors, cells and/or compositions provided herein, against a disease or condition, e.g., a cancer, such as any described herein.

Immune checkpoint inhibitors include any agent that blocks or inhibits in a statistically significant manner, the inhibitory pathways of the immune system. Such inhibitors may include small molecule inhibitors or may include antibodies, or antigen binding fragments thereof, that bind to and block or inhibit immune checkpoint receptors, ligands and/or receptor-ligand interaction. In some embodiments, modulation, enhancement and/or stimulation of particular receptors can overcome immune checkpoint pathway components. Illustrative immune checkpoint molecules that may be targeted for blocking, inhibition, modulation, enhancement and/or stimulation include, but are not limited to, PD-1 (CD279), PD-L1 (CD274, B7-H1), PDL2 (CD273, B7-DC), CTLA-4, LAG-3 (CD223), TIM-3, 4-1BB (CD137), 4-1BBL (CD137L), GITR (TNFRSFi8, AITR), CD40, OX40 (CD134, TNFRSF4), CXCR2, tumor associated antigens (TAA), B7-H3, B7-H4, BTLA, HVEM, GAL9, B7H3, B7H4, VISTA, KIR, 2B4 (belongs to the CD2 family of molecules and is expressed on all NK, γδ, and memory CD8+ (ap) T cells), CD160 (also referred to as BY55), CGEN-15049, CEACAM (e.g., CEACAM-1, CEACAM-3 and/or CEACAM-5), TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, and a transforming growth factor receptor (TGFR; e.g., TGFR beta). Immune checkpoint inhibitors include antibodies, or antigen binding fragments thereof, or other binding proteins that bind to and block or inhibit and/or enhance or stimulate the activity of one or more of any of the said molecules.

Exemplary immune checkpoint inhibitors include Tremelimumab (CTLA-4 blocking antibody, also known as ticilimumab, CP-675,206), anti-OX40, PD-L1 monoclonal antibody (Anti-B7-H1; MEDI4736), MK-3475 (PD-1 blocker), nivolumab (anti-PD-1 antibody), CT-011 (anti-PD-1 antibody), BY55 monoclonal antibody, AMP224 (anti-PD-L1 antibody), BMS-936559 (anti-PD-L1 antibody), MPLDL3280A (anti-PD-L1 antibody), MSB0010718C (anti-PD-L1 antibody) and ipilimumab (anti-CTLA-4 antibody, also known as Yervoy®, MDX-010 and MDX-101). Exemplary of immunomodulatory antibodies include, but are not limited to, Daclizumab (Zenapax), Bevacizumab (Avastin®), Basiliximab, Ipilimumab, Nivolumab, pembrolizumab, MPDL3280A, Pidilizumab (CT-011), MK-3475, BMS-936559, MPDL3280A (Atezolizumab), tremelimumab, IMP321, BMS-986016, LAG525, urelumab, PF-05082566, TRX518, MK-4166, dacetuzumab (SGN-40), lucatumumab (HCD122), SEA-CD40, CP-870, CP-893, MEDI6469, MEDI6383, MOXRO916, AMP-224, MSB0010718C (Avelumab), MEDI4736, PDR001, rHIgM12B7, Ulocuplumab, BKT140, Varlilumab (CDX-1127), ARGX-110, MGA271, lirilumab (BMS-986015, IPH2101), IPH2201, ARGX-115, Emactuzumab, CC-90002 and MNRP1685A or an antibody-binding fragment thereof. Other exemplary immunomodulators include, e.g., afutuzumab (available from Roche®); pegfilgrastim (Neulasta®); lenalidomide (CC-5013, Revlimid®); thalidomide (Thalomid®), actimid (CC4047); and IRX-2 (mixture of human cytokines including interleukin 1, interleukin 2, and interferon gamma, CAS 951209-71-5, available from IRX Therapeutics).

Programmed cell death 1 (PD-1) is an immune checkpoint protein that is expressed in B cells, NK cells, and T cells (Shinohara et al., 1995, Genomics 23:704-6; Blank et al., 2007, Cancer Immunol Immunother 56:739-45; Finger et al., 1997, Gene 197:177-87; Pardoll (2012) Nature Reviews Cancer 12:252-264). The major role of PD-1 is to limit the activity of T cells in peripheral tissues during inflammation in response to infection, as well as to limit autoimmunity. PD-1 expression is induced in activated T cells and binding of PD-1 to one of its endogenous ligands acts to inhibit T-cell activation by inhibiting stimulatory kinases. PD-1 also acts to inhibit the TCR “stop signal”. PD-1 is highly expressed on Treg cells and may increase their proliferation in the presence of ligand (Pardoll (2012) Nature Reviews Cancer 12:252-264). Anti-PD 1 antibodies have been used for treatment of melanoma, non-small-cell lung cancer, bladder cancer, prostate cancer, colorectal cancer, head and neck cancer, triple-negative breast cancer, leukemia, lymphoma and renal cell cancer (Topalian et al., 2012, N Engl J Med 366:2443-54; Lipson et al., 2013, Clin Cancer Res 19:462-8; Berger et al., 2008, Clin Cancer Res 14:3044-51; Gildener-Leapman et al., 2013, Oral Oncol 49:1089-96; Menzies & Long, 2013, Ther Adv Med Oncol 5:278-85). Exemplary anti-PD-1 antibodies include nivolumab (Opdivo by BMS), pembrolizumab (Keytruda by Merck), pidilizumab (CT-011 by Cure Tech), lambrolizumab (MK-3475 by Merck), and AMP-224 (Merck), nivolumab (also referred to as Opdivo, BMS-936558 or MDX1106; Bristol-Myers Squibb) is a fully human IgG4 monoclonal antibody which specifically blocks PD-1. Nivolumab (clone 5C4) and other human monoclonal antibodies that specifically bind to PD-1 are described in U.S. Pat. No. 8,008,449 and WO2006/121168. Pidilizumab (CT-011; Cure Tech) is a humanized IgG1k monoclonal antibody that binds to PD-1. Pidilizumab and other humanized anti-PD-1 monoclonal antibodies are described in WO2009/101611. Pembrolizumab (formerly known as lambrolizumab, and also referred to as Keytruda, MK03475; Merck) is a humanized IgG4 monoclonal antibody that binds to PD-1. Pembrolizumab and other humanized anti-PD-1 antibodies are described in U.S. Pat. No. 8,354,509 and WO2009/114335. Other anti-PD-1 antibodies include AMP 514 (Amplimmune), among others, e.g., anti-PD-1 antibodies described in U.S. Pat. No. 8,609,089, US 2010028330, US 20120114649 and/or US 20150210769. AMP-224 (B7-DCIg; Amplimmune; e.g., described in WO2010/027827 and WO2011/066342), is a PD-L2 Fc fusion soluble receptor that blocks the interaction between PD-1 and B7-H1.

PD-L1 (also known as CD274 and B7-H1) and PD-L2 (also known as CD273 and B7-DC) are ligands for PD-1, found on activated T cells, B cells, myeloid cells, macrophages, and some types of tumor cells. Anti-tumor therapies have focused on anti-PD-L1 antibodies. The complex of PD-1 and PD-L1 inhibits proliferation of CD8+ T cells and reduces the immune response (Topalian et al., 2012, N Engl J Med 366:2443-54; Brahmer et al., 2012, N Eng J Med 366:2455-65). Anti-PD-L1 antibodies have been used for treatment of non-small cell lung cancer, melanoma, colorectal cancer, renal-cell cancer, pancreatic cancer, gastric cancer, ovarian cancer, breast cancer, and hematologic malignancies (Brahmer et al., 2012, N Eng J Med 366:2455-65; Ott et al., 2013, Clin Cancer Res 19:5300-9; Radvanyi et al., 2013, Clin Cancer Res 19:5541; Menzies & Long, 2013, Ther Adv Med Oncol 5:278-85; Berger et al., 2008, Clin Cancer Res 14:13044-51). Exemplary anti-PD-L1 antibodies include MDX-1105 (Medarex), MEDI4736 (Medimmune) MPDL3280A (Genentech), BMS-935559 (Bristol-Myers Squibb) and MSB0010718C. MEDI4736 (Medimmune) is a human monoclonal antibody that binds to PD-L1, and inhibits interaction of the ligand with PD-1. MDPL3280A (Genentech/Roche) is a human Fe optimized IgG1 monoclonal antibody that binds to PD-L1. MDPL3280A and other human monoclonal antibodies to PD-L1 are described in U.S. Pat. No. 7,943,743 and U.S Publication No. 20120039906. Other anti-PD-L1 binding agents include YW243.55.570 (see WO2010/077634) and MDX-1105 (also referred to as BMS-936559, and, e.g., anti-PD-L1 binding agents described in WO2007/005874).

Cytotoxic T-lymphocyte-associated antigen (CTLA-4), also known as CD152, is a co-inhibitory molecule that functions to regulate T-cell activation. CTLA-4 is a member of the immunoglobulin superfamily that is expressed exclusively on T-cells. CTLA-4 acts to inhibit T-cell activation and is reported to inhibit helper T-cell activity and enhance regulatory T-cell immunosuppressive activity. Although the precise mechanism of action of CTLA-4 remains under investigation, it has been suggested that it inhibits T cell activation by outcompeting CD28 in binding to CD80 and CD86, as well as actively delivering inhibitor signals to the T cell (Pardoll (2012) Nature Reviews Cancer 12:252-264). Anti-CTLA-4 antibodies have been used in clinical trials for the treatment of melanoma, prostate cancer, small cell lung cancer, non-small cell lung cancer (Robert & Ghiringhelli, 2009, Oncologist 14:848-61; Ott et al., 2013, Clin Cancer Res 19:5300; Weber, 2007, Oncologist 12:864-72; Wada et al., 2013, J Transl Med 11:89). A significant feature of anti-CTLA-4 is the kinetics of anti-tumor effect, with a lag period of up to 6 months after initial treatment required for physiologic response. In some cases, tumors may actually increase in size after treatment initiation, before a reduction is seen (Pardoll (2012) Nature Reviews Cancer 12:252-264). Exemplary anti-CTLA-4 antibodies include ipilimumab (Bristol-Myers Squibb) and tremelimumab (Pfizer). Ipilimumab has recently received FDA approval for treatment of metastatic melanoma (Wada et al., 2013, J Transl Med 11:89).

Lymphocyte activation gene-3 (LAG-3), also known as CD223, is another immune checkpoint protein. LAG-3 has been associated with the inhibition of lymphocyte activity and in some cases the induction of lymphocyte anergy. LAG-3 is expressed on various cells in the immune system including B cells, NK cells, and dendritic cells. LAG-3 is a natural ligand for the MHC class II receptor, which is substantially expressed on melanoma-infiltrating T cells including those endowed with potent immune-suppressive activity. Exemplary anti-LAG-3 antibodies include BMS-986016 (Bristol-Myers Squib), which is a monoclonal antibody that targets LAG-3. IMP701 (Immutep) is an antagonist LAG-3 antibody and IMP731 (Immutep and GlaxoSmithKline) is a depleting LAG-3 antibody. Other LAG-3 inhibitors include IMP321 (Immutep), which is a recombinant fusion protein of a soluble portion of LAG-3 and Ig that binds to MHC class II molecules and activates antigen presenting cells (APC). Other antibodies are described, e.g., in WO2010/019570 and US 2015/0259420

T-cell immunoglobulin domain and mucin domain-3 (TIM-3), initially identified on activated Th1 cells, has been shown to be a negative regulator of the immune response. Blockade of TIM-3 promotes T-cell mediated anti-tumor immunity and has anti-tumor activity in a range of mouse tumor models. Combinations of TIM-3 blockade with other immunotherapeutic agents such as TSR-042, anti-CD137 antibodies and others, can be additive or synergistic in increasing anti-tumor effects. TIM-3 expression has been associated with a number of different tumor types including melanoma, NSCLC and renal cancer, and additionally, expression of intratumoral TIM-3 has been shown to correlate with poor prognosis across a range of tumor types including NSCLC, cervical, and gastric cancers. Blockade of TIM-3 is also of interest in promoting increased immunity to a number of chronic viral diseases. TIM-3 has also been shown to interact with a number of ligands including galectin-9, phosphatidylserine and HMGB1, although which of these, if any, are relevant in regulation of anti-tumor responses is not clear at present. In some embodiments, antibodies, antibody fragments, small molecules, or peptide inhibitors that target TIM-3 can bind to the IgV domain of TIM-3 to inhibit interaction with its ligands. Exemplary antibodies and peptides that inhibit TIM-3 are described in US 2015/0218274, WO2013/006490 and US 2010/0247521. Other anti-TIM-3 antibodies include humanized versions of RMT3-23 (Ngiow et al., 2011, Cancer Res, 71:3540-3551), and clone 8B.2C12 (Monney et al., 2002, Nature, 415:536-541). Bi-specific antibodies that inhibit TIM-3 and PD-1 are described in US 2013/0156774.

In some embodiments, the additional agent is a CEACAM inhibitor (e.g., CEACAM-1, CEACAM-3, and/or CEACAM-5 inhibitor). In some embodiments, the inhibitor of CEACAM is an anti-CEACAM antibody molecule. Exemplary anti-CEACAM-1 antibodies are described in WO 2010/125571, WO 2013/082366 WO 2014/059251 and WO 2014/022332, e.g., a monoclonal antibody 34B1, 26H7, and 5F4; or a recombinant form thereof, as described in, e.g., US 2004/0047858, U.S. Pat. No. 7,132,255 and WO 99/052552. In some embodiments, the anti-CEACAM antibody binds to CEACAM-5 as described in, e.g., Zheng et al. PLoS One. (2011) 6(6): e21146), or cross-reacts with CEACAM-1 and CEACAM-5 as described in, e.g., WO 2013/054331 and US 2014/0271618.

4-1BB, also known as CD137, is transmembrane glycoprotein belonging to the TNFR superfamily. 4-1BB receptors are present on activated T cells and B cells and monocytes. An exemplary anti-4-1BB antibody is urelumab (BMS-663513), which has potential immunostimulatory and antineoplastic activities.

Tumor necrosis factor receptor superfamily, member 4 (TNFRSF4), also known as OX40 and CD134, is another member of the TNFR superfamily. OX40 is not constitutively expressed on resting naïve T cells and acts as a secondary co-stimulatory immune checkpoint molecule. Exemplary anti-OX40 antibodies are MEDI6469 and MOXRO916 (RG7888, Genentech).

In some embodiments, the additional agent includes a molecule that decreases the regulatory T cell (Treg) population. Methods that decrease the number of (e.g., deplete) Treg cells are known and include, e.g., CD25 depletion, cyclophosphamide administration, and modulating Glucocorticoid-induced TNFR family related gene (GITR) function. GITR is a member of the TNFR superfamily that is upregulated on activated T cells, which enhances the immune system. Reducing the number of Treg cells in a subject prior to apheresis or prior to administration of engineered cells, e.g., CAR-expressing cells, can reduce the number of unwanted immune cells (e.g., Tregs) in the tumor microenvironment and reduces the subject's risk of relapse. In some embodiments, the additional agent includes a molecule targeting GITR and/or modulating GITR functions, such as a GITR agonist and/or a GITR antibody that depletes regulatory T cells (Tregs). In some embodiments, the additional agent includes cyclophosphamide. In some embodiments, the GITR binding molecule and/or molecule modulating GITR function (e.g., GITR agonist and/or Treg depleting GITR antibodies) is administered prior to the engineered cells, e.g., CAR-expressing cells. For example, in some embodiments, the GITR agonist can be administered prior to apheresis of the cells. In some embodiments, cyclophosphamide is administered to the subject prior to administration (e.g., infusion or re-infusion) of the engineered cells, e.g., CAR-expressing cells or prior to apheresis of the cells. In some embodiments, cyclophosphamide and an anti-GITR antibody are administered to the subject prior to administration (e.g., infusion or re-infusion) of the engineered cells, e.g., CAR-expressing cells or prior to apheresis of the cells.

In some embodiments, the additional agent is a GITR agonist. Exemplary GITR agonists include, e.g., GITR fusion proteins and anti-GITR antibodies (e.g., bivalent anti-GITR antibodies) such as, e.g., a GITR fusion protein described in U.S. Pat. No. 6,111,090, European Patent No. 090505B 1, U.S. Pat. No. 8,586,023, PCT Publication Nos.: WO 2010/003118 and 2011/090754, or an anti-GITR antibody described, e.g., in U.S. Pat. No. 7,025,962, European Patent No. 1947183B 1, U.S. Pat. Nos. 7,812,135, 8,388,967, 8,591,886, European Patent No. EP 1866339, WO 2011/028683, WO 2013/039954, WO2005/007190, WO 2007/133822, WO2005/055808, WO 99/40196, WO 2001/03720, WO99/20758, WO2006/083289, WO 2005/115451, U.S. Pat. No. 7,618,632, and WO 2011/051726. An exemplary anti-GITR antibody is TRX518.

In some embodiments, the additional agent enhances tumor infiltration or transmigration of the administered cells, e.g., CAR-expressing cells. For example, in some embodiments, the additional agent stimulates CD40, such as CD40L, e.g., recombinant human CD40L. Cluster of differentiation 40 (CD40) is also a member of the TNFR superfamily. CD40 is a costimulatory protein found on antigen-presenting cells and mediates a broad variety of immune and inflammatory responses. CD40 is also expressed on some malignancies, where it promotes proliferation. Exemplary anti-CD40 antibodies are dacetuzumab (SGN-40), lucatumumab (Novartis, antagonist), SEA-CD40 (Seattle Genetics), and CP-870,893. In some embodiments, the additional agent that enhances tumor infiltration includes tyrosine kinase inhibitor sunitnib, heparanase, and/or chemokine receptors such as CCR2, CCR4, and CCR7.

In some embodiments, the additional agent is a structural or functional analog or derivative of thalidomide and/or an inhibitor of E3 ubiquitin ligase. In some embodiments, the immunomodulatory agent binds to cereblon (CRBN). In some embodiments, the immunomodulatory agent binds to the CRBN E3 ubiquitin-ligase complex. In some embodiments, the immunomodulatory agent binds to CRBN and the CRBN E3 ubiquitin-ligase complex. In some embodiments, the immunomodulatory agent up-regulates the protein or gene expression of CRBN. In some aspects, CRBN is the substrate adaptor for the CRL4^{CRBN} E3 ubiquitin ligase, and modulates the specificity of the enzyme. In some embodiments, binding to CRB or the CRBN E3 ubiquitin ligase complex inhibits E3 ubiquitin ligase activity. In some embodiments, the immunomodulatory agent induces the ubiquitination of KZF1 (Ikaros) and IKZF3 (Aiolos) and/or induces degradation of IKZF1 (Ikaros) and IKZF3 (Aiolos). In some embodiments, the immunomodulatory agent induces the ubiquitination of casein kinase 1A1 (CK1α) by the CRL4^{CRBN} E3 ubiquitin ligase. In some embodiments, the ubiquitination of CK1α results in CK1α degradation.

In some embodiments, the additional agent is an inhibitor of the Ikaros (IKZF1) transcription factor. In some embodiments, the additional agent enhances ubiquitination of Ikaros. In some embodiments, the additional agent enhances the degradation of Ikaros. In some embodiments, the additional agent down-regulates the protein or gene expression of Ikaros. In some embodiments, administration of the additional agent causes a decrease in Ikaros protein levels.

In some embodiments, the additional agent is an inhibitor of the Aiolos (IKZF3) transcription factor. In some embodiments, the additional agent enhances ubiquitination of Aiolos. In some embodiments, the additional agent enhances the degradation of Aiolos. In some embodiments, the additional agent down-regulates the protein or gene expression of Aiolos. In some embodiments, administration of the additional agent causes a decrease in Aiolos protein levels.

In some embodiments, the additional agent is an inhibitor of both the Ikaros (IKZF1) and Aiolos (IKZF3) transcription factors. In some embodiments, the additional agent enhances ubiquitination of both Ikaros and Aiolos. In some embodiments, the additional agent enhances the degradation of both Ikaros and Aiolos. In some embodiments, the additional agent enhances ubiquitination and degradation of both Ikaros and Aiolos. In some embodiments, administration of the additional agent causes both Aiolos protein levels and Ikaros protein levels to decrease.

In some embodiments, the additional agent is a selective cytokine inhibitory drug (SelCID). In some embodiments, the additional agent inhibits the activity of phosphodiesterase-4 (PDE4). In some embodiments, the additional agent suppresses the enzymatic activity of the CDC25 phosphatases. In some embodiments, the additional agent alters the intracellular trafficking of CDC25 phosphatases.

In some embodiments, the additional agent is thalidomide (2-(2,6-dioxopiperidin-3-yl)-1H-isoindole-1,3(2H)-dione) or an analog or derivative of thalidomide. In certain embodiments, a thalidomide derivative includes structural variants of thalidomide that have a similar biological activity. Exemplary thalidomide derivatives include, but are not limited to lenalidomide (REVLIMMUNOMODULATORY COMPOUND™; Celgene Corporation), pomalidomide (also known as ACTIMMUNOMODULATORY COMPOUND™ or POMALYST™ (Celgene Corporation)), CC-1088, CDC-501, and CDC-801, and the compounds disclosed in U.S. Pat. Nos. 5,712,291; 7,320,991; and 8,716,315; U.S. Appl. No. 2016/0313300; and PCT Pub. Nos. WO 2002/068414 and WO 2008/154252.

In some embodiments, the additional agent is 1-oxo- and 1,3 dioxo-2-(2,6-dioxopiperldin-3-yl) isoindolines substituted with amino in the benzo ring as described in U.S. Pat. No. 5,635,517 which is incorporated herein by reference.

In some embodiments, the additional agent is a compound of the following formula:

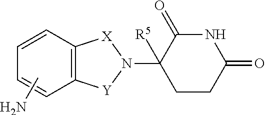

wherein one of X and Y is —C(O)— and the other of X and Y is —C(O)— or —CH₂—, and R⁵ is hydrogen or lower alkyl, or a pharmaceutically acceptable salt thereof. In some embodiments, X is —C(O)— and Y is —CH₂—. In some embodiments, both X and Y are —C(O)—. In some embodiments, R⁵ is hydrogen. In other embodiments, R⁵ is methyl.

In some embodiments, the additional agent is a compound that belongs to a class of substituted 2-(2, 6-dioxopiperidin-3-yl)phthalate immunomodulatory compounds and substituted 2-(2,6-dioxopiperldin-3-yl)-1-oxoisoindoles, such as those described in U.S. Pat. Nos. 6,281,230; 6,316,471; 6,335,349; and 6,476,052, and International Patent Application No. PCT/US97/13375 (International Publication No. WO 98/03502), each of which is incorporated herein by reference.

In some embodiments, the additional agent is a compound of the following formula:

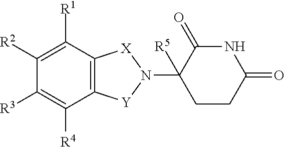

wherein
one of X and Y is —C(O)— and the other of X and Y is —C(O)— or —CH₂—;
(1) each of R¹, R², R³, and R⁴ are independently halo, alkyl of 1 to 4 carbon atoms, or alkoxy or 1 to 4 carbon atoms, or
(2) one of R¹, R³, R⁴, and R⁵ is —NHR^{a} and the remaining of R¹, R², R³, and R⁴ is are hydrogen, wherein R^{a} is hydrogen or alkyl of 1 to 8 carbon atoms;
R⁵ is hydrogen or alkyl of 1 to 8 carbon atoms, benzyl, or halo;
provided that R⁵ is other than hydrogen if X and Y are —C(O)— and (i) each of R¹, R², R³, and R⁴ is fluoro; or (ii) one of R¹, R², R³, and R⁴ is amino;
or a pharmaceutically acceptable salt thereof.

In some embodiments, the additional agent is a compound that belongs to a class of isoindole-immunomodulatory compounds disclosed in U.S. Pat. No. 7,091,353, U.S. Patent Publication No. 2003/0045552, and International Application No. PCT/USOI/50401 (International Publication No. WO02/059106), each of which are incorporated herein by reference. For example, in some embodiments, the additional agent is [2-(2,6-dioxo-piperidin-3-yl)-1,3-dioxo-2,3-dihydro-1H-isoindol-4-ylmethyl]-amide; (2-(2,6-dioxo-piperidin-3-yl)-1,3-dioxo-2,3-dihydro-1H-isoindol-4-ylmethyl)-carbamic acid tert-butyl ester; 4-(aminomethyl)-2-(2,6-dioxo(3-piperidyl))-isoindoline-1,3-dione; N-(2-(2,6-dioxo-piperidin-3-yl)-1,3-dioxo-2,3-dihydro-1H-isoindol-4-ylmethyl)-acetamide; N-{(2-(2,6-dioxo(3-piperidyl)-1,3-dioxoisoindolin-4-yl)methyl}cyclopropyl-carboxamide; 2-chloro-N-{(2-(2,6-dioxo(3-piperidyl))-1,3-dioxoisoindolin-4-yl)methyl}acetamide; N-(2-(2,6-dioxo(3-piperidyl))-1,3-dioxoisoindolin-4-yl)-3-pyridylcarboxamide; 3-{1-oxo-4-(benzylamino)isoindolin-2-yl}piperidine-2,6-dione; 2-(2,6-dioxo(3-piperidyl))-4-(benzylamino)isoindoline-1,3-dione; N-{(2-(2,6-dioxo(3-piperidyl))-1,3-dioxoisoindolin-4-yl)methyl}propanamide; N-{(2-(2,6-dioxo(3-piperidyl))-1,3-dioxoisoindolin-4-yl)methyl}-3-pyridylcarboxamide; N-{(2-(2,6-dioxo(3-piperidyl))-1,3-dioxoisoindolin-4-yl)methyl}heptanamide; N-{(2-(2,6-dioxo(3-piperidyl))-1,3-dioxoisoindolin-4-yl)methyl}-2-furylcarboxamide; {N-(2-(2,6-dioxo(3-piperidyl))-1,3-dioxoisoindolin-4-yl)carbamoyl}methyl acetate; N-(2-(2,6-dioxo(3-piperidyl))-1,3-dioxoisoindolin-4-yl)pentanamide; N-(2-(2,6-dioxo(3-piperidyl))-1,3-dioxoisoindolin-4-yl)-2-thienylcarboxamide; N-{[2-(2,6-dioxo(3-piperidyl))-1,3-dioxoisoindolin-4-yl]methyl}(butylamino)carboxamide; N-{[2-(2,6-dioxo(3-piperidyl))-1,3-dioxoisoindolin-4-yl]methyl}(octylamino)carboxamide; or N-{[2-(2,6-dioxo(3-piperidyl))-1,3-dioxoisoindolin-4-yl]methyl}(benzylamino)carboxamide.

In some embodiments, the additional agent is a compound that belongs to a class of isoindole-immunomodulatory compounds disclosed in U.S. Patent Application Publication Nos. 2002/0045643, International Publication No. WO 98/54170, and U.S. Pat. No. 6,395,754, each of which is incorporated herein by reference. In some embodiments, the additional agent is a tetra substituted 2-(2,6-dioxopiperdin-3-yl)-1-oxoisoindolines described in U.S. Pat. No. 5,798,368, which is incorporated herein by reference. In some embodiments, the additional agent is 1-oxo and 1,3-dioxo-2-(2,6-dioxopiperidin-3-yl) isoindolines disclosed in U.S. Pat. No. 6,403,613, which is incorporated herein by reference. In some embodiments the additional agent is a 1-oxo or 1,3-dioxoisoindoline substituted in the 4- or 5-position of the indoline ring as described in U.S. Pat. Nos. 6,380,239 and 7,244,759, both of which are incorporated herein by reference.

In some embodiments, the additional agent is 2-(4-amino-1-oxo-1,3-dihydro-isoindol-2-yl)-4-carbamoyl-butyric acid or 4-(4-amino-1-oxo-1,3-dihydro-isoindol-2-yl)-4-carbamoyl-butyric acid. In some embodiments, the immunomodulatory compound is 4-carbamoyl-4-{4-[(furan-2-yl-methyl)-amino]-1,3-dioxo-1,3-dihydro-isoindol-2-yl}-butyric acid, 4-carbamoyl-2-{4-[(furan-2-yl-methyl)-amino]-1,3-dioxo-1,3-dihydro-isoindol-2-yl}-butyric acid, 2-{4-[(furan-2-yl-methyl)-amino]-1,3-dioxo-1,3-dihydro-isoindol-2-yl}-4-phenylcarbamoyl-butyric acid, or 2-{4-[(furan-2-yl-methyl)-amino]-1,3-dioxo-1,3-dihydro-isoindol-2-yl}-pentanedioic acid.

In some embodiments, the additional agent is a isoindoline-1-one or isoindoline-1,3-dione substituted in the 2-position with 2,6-dioxo-3-hydroxypiperidin-5-yl as described in U.S. Pat. No. 6,458,810, which is incorporated herein by reference. In some embodiments, the immunomodulatory compound is 3-(5-amino-2-methyl-4-oxo-4H-quinazolin-3-yl)-piperidine-2,6-dione, or an enantiomer or a mixture of enantiomers thereof; or a pharmaceutically acceptable salt, solvate, hydrate, co-crystal, clathrate, or polymorph thereof. In some embodiments, the immunomodulatory compound is 3-[4-(4-morpholin-4-ylmethyl-benzyloxy)-1-oxo-1,3-dihydro-isoindol-2-yl]-piperidine-2,6-dione.

In some embodiments, the additional agent is as described in Oshima, K. et al., Nihon Rinsho., 72(6):1130-5 (2014); Millrine, D. et al., Trends Mol Med., 23(4):348-364 (2017); and Collins, et al., Biochem J., 474(7):1127-1147 (2017).

In some embodiments, the additional agent includes thalidomide drugs or analogs thereof and/or derivatives thereof, such as lenalidomide, pomalidomide or apremilast. See, e.g., Bertilaccio et al., Blood (2013) 122:4171, Otahal et al., Oncoimmunology (2016) 5(4):e1115940; Fecteau et al., Blood (2014) 124(10):1637-1644 and Kuramitsu et al., Cancer Gene Therapy (2015) 22:487-495). Lenalidomide ((RS)-3-(4-Amino-1-oxo-1,3-dihydro-2H-isoindol-2-yl)piperidine-2,6-dione; also known as Revlimid) is a synthetic derivative of thalidomide, and has multiple immunomodulatory effects, including enforcement of immune synapse formation between T cell and antigen presenting cells (APCs). In some embodiments, the additional agent is lenalidomide, pomalidomide, avadomide, a stereoisomer of lenalidomide, pomalidomide, avadomide or a pharmaceutically acceptable salt, solvate, hydrate, co-crystal, clathrate, or polymorph thereof. In some embodiments, the immunomodulatory compound is lenalidomide, a stereoisomer of lenalidomide or a pharmaceutically acceptable salt, solvate, hydrate, co-crystal, clathrate, or polymorph thereof. In some embodiments, the immunomodulatory compound is lenalidomide, or ((RS)-3-(4-Amino-1-oxo-1,3-dihydro-2H-isoindol-2-yl)piperidine-2,6-dione).

In certain embodiments, the lesion is disrupted by administering the thalidomide derivative lenalidomide, ((RS)-3-(4-Amino-1-oxo-1,3-dihydro-2H-isoindol-2-yl)piperidine-2,6-dione) to the subject. Lenalidomide is FDA approved for the treatment of multiple myeloma, myelodysplastic syndrome associated with deletion 5q, and most recently in relapsed/refractory mantle-cell lymphoma (MCL). Lenalidomide generally is a synthetic derivative of thalidomide, and is currently understood to have multiple immunomodulatory effects, including enforcement of immune synapse formation between T cell and antigen presenting cells (APCs). For example, in some cases, lenalidomide modulates T cell responses and results in increased interleukin (IL)-2 production in CD4+ and CD8+ T cells, induces the shift of T helper (Th) responses from Th2 to Th1, inhibits expansion of regulatory subset of T cells (Tregs), and improves functioning of immunological synapses in follicular lymphoma and chronic lymphocytic leukemia (CLL) (Otahal et al., Oncoimmunology (2016) 5(4):e1115940). Lenalidomide also has direct tumoricidal activity in patients with multiple myeloma (MM) and directly and indirectly modulates survival of CLL tumor cells by affecting supportive cells, such as nurse-like cells found in the microenvironment of lymphoid tissues. Lenalidomide also can enhance T-cell proliferation and interferon-y production in response to activation of T cells via CD3 ligation or dendritic cell-mediated activation. In addition, lenalidomide is thought to decrease proliferation of pro-inflammatory cytokines including TNF-a, IL-1, IL-6, and IL-12 and enhance antibody-dependent cellular cytotoxicity (ADCC) via increased NK cell activation. Lenalidomide can also induce malignant B cells to express higher levels of immunostimulatory molecules such as CD80, CD86, HLA-DR, CD95, and CD40 (Fecteau et al., Blood (2014) 124(10):1637-1644). Cereblon, an E3 ubiquitin ligase, was identified as the primary target for thalidomide-induced teratogenesis (Ito et al., T., (2010) Science 327: 1345-1350). Lenalidomide also targets cereblon and it has been shown that this leads to the reduction of c-Myc and IRF4 expression while also increasing expression of p21 that leads to G1 cell-cycle arrest (Lopez-Girona et al., (2012) Leukemia 26: 2326-2335).

In some embodiments, the additional agent is a B-cell inhibitor. In some embodiments, the additional agent is one or more B-cell inhibitors selected from among inhibitors of CD10, CD19, CD20, CD22, CD34, CD123, CD79a, CD79b, CD179b, FLT-3, or ROR1, or a combination thereof. In some embodiments, the B-cell inhibitor is an antibody (e.g., a mono- or bispecific antibody) or an antigen binding fragment thereof. In some embodiments, the additional agent is an engineered cell expressing recombinant receptors that target B-cell targets, e.g., CD10, CD19, CD20, CD22, CD34, CD123, CD79a, CD79b, CD179b, FLT-3, or ROR1.

In some embodiments, the additional agent is a CD20 inhibitor, e.g., an anti-CD20 antibody (e.g., an anti-CD20 mono- or bi-specific antibody) or a fragment thereof. Exemplary anti-CD20 antibodies include but are not limited to rituximab, ofatumumab, ocrelizumab (also known as GA101 or R05072759), veltuzumab, obinutuzumab, TRU-015 (Trubion Pharmaceuticals), ocaratuzumab (also known as AME-133v or ocaratuzumab), and Pro131921 (Genentech). See, e.g., Lim et al. Haematologica. (2010) 95(1):135-43. In some embodiments, the anti-CD20 antibody comprises rituximab. Rituximab is a chimeric mouse/human monoclonal antibody IgG1 kappa that binds to CD20 and causes cytolysis of a CD20 expressing cell. In some embodiments, the additional agent includes rituximab. In some embodiments, the CD20 inhibitor is a small molecule.

In some embodiments, the additional agent is a CD22 inhibitor, e.g., an anti-CD22 antibody (e.g., an anti-CD22 mono- or bi-specific antibody) or a fragment thereof. Exemplary anti-CD22 antibodies include epratuzumab and RFB4. In some embodiments, the CD22 inhibitor is a small molecule. In some embodiments, the antibody is a monospecific antibody, optionally conjugated to a second agent such as a chemotherapeutic agent. For instance, in some embodiments, the antibody is an anti-CD22 monoclonal antibody-MMAE conjugate (e.g., DCDT2980S). In some embodiments, the antibody is an scFv of an anti-CD22 antibody, e.g., an scFv of antibody RFB4. In some embodiments, the scFv is fused to all of or a fragment of Pseudomonas exotoxin-A (e.g., BL22). In some embodiments, the scFv is fused to all of or a fragment of (e.g., a 38 kDa fragment of) Pseudomonas exotoxin-A (e.g., moxetumomab pasudotox). In some embodiments, the anti-CD22 antibody is an anti-CD19/CD22 bispecific antibody, optionally conjugated to a toxin. For instance, in some embodiments, the anti-CD22 antibody comprises an anti-CD19/CD22 bispecific portion, (e.g., two scFv ligands, recognizing human CD19 and CD22) optionally linked to all of or a portion of diphtheria toxin (DT), e.g., first 389 amino acids of diphtheria toxin (DT), DT 390, e.g., a ligand-directed toxin such as DT2219ARL). In some embodiments, the bispecific portion (e.g., anti-CD 19/anti-CD22) is linked to a toxin such as deglycosylated ricin A chain (e.g., Combotox).

In some embodiments, the immunomodulatory agent is a cytokine. In some embodiments, the immunomodulatory agent is a cytokine or is an agent that induces increased expression of a cytokine in the tumor microenvironment. Cytokines have important functions related to T cell expansion, differentiation, survival, and homeostasis. Cytokines that can be administered to the subject receiving the binding molecules (e.g., ROR1-binding molecules), recombinant receptors, cells and/or compositions provided herein include one or more of IL-2, IL-4, IL-7, IL-9, IL-15, IL-18, and IL-21. In some embodiments, the cytokine administered is IL-7, IL-15, or IL-21, or a combination thereof. In some embodiments, administration of the cytokine to the subject that has sub-optimal response to the administration of the engineered cells, e.g., CAR-expressing cells improves efficacy and/or anti-tumor activity of the administered cells, e.g., CAR-expressing cells.

By “cytokine” is meant a generic term for proteins released by one cell population that act on another cell as intercellular mediators. Examples of such cytokines are lymphokines, monokines, and traditional polypeptide hormones. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-alpha and -beta; mullerian-inhibiting substance; mouse gonadotropin-associated peptide; inhibin; activin; vascular endothelial growth factor; integrin; thrombopoietin (TPO); nerve growth factors such as NGF-beta; platelet-growth factor; transforming growth factors (TGFs) such as TGF-alpha and TGF-beta; insulin-like growth factor-I and -II; erythropoietin (EPO); osteoinductive factors; interferons such as interferon-alpha, beta, and -gamma; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-lalpha, IL-2, IL-3, IL-4, IL-5, IL-6, IL-7, IL-8, IL-9, IL-10, IL-11, IL-12; IL-15, a tumor necrosis factor such as TNF-alpha or TNF-beta; and other polypeptide factors including LIF and kit ligand (KL). As used herein, the term cytokine includes proteins from natural sources or from recombinant cell culture, and biologically active equivalents of the native sequence cytokines. For example, the immunomodulatory agent is a cytokine and the cytokine is IL-4, TNF-α, GM-CSF or IL-2.

In some embodiments, the additional agent includes an interleukin-15 (IL-15) polypeptide, an interleukin-15 receptor alpha (IL-15Ra) polypeptide, or combination thereof, e.g., hetIL-15 (Admune Therapeutics, LLC). hetIL-15 is a heterodimeric non-covalent complex of IL-15 and IL-15Ra. hetIL-15 is described in, e.g., U.S. Pat. No. 8,124,084, U.S. 2012/0177598, U.S. 2009/0082299, U.S. 2012/0141413, and U.S. 2011/0081311. In some embodiments, the immunomodulatory agent can contain one or more cytokines. For example, the interleukin can include leukocyte interleukin injection (Multikine), which is a combination of natural cytokines. In some embodiments, the immunomodulatory agent is a Toll-like receptor (TLR) agonist, an adjuvant or a cytokine.

In some embodiments, the additional agent is an agent that ameliorates or neutralizes one or more toxicities or side effects associated with the cell therapy. In some embodiments, the additional agent is selected from among a steroid (e.g., corticosteroid), an inhibitor of TNFα, and an inhibitor of IL-6. An example of a TNFα inhibitor is an anti-TNFα antibody molecule such as, infliximab, adalimumab, certolizumab pegol, and golimumab. Another example of a TNFα inhibitor is a fusion protein such as entanercept. Small molecule inhibitors of TNFα include, but are not limited to, xanthine derivatives (e.g. pentoxifylline) and bupropion. An example of an IL-6 inhibitor is an anti-IL-6 antibody molecule such as tocilizumab, sarilumab, elsilimomab, CNTO 328, ALD518/BMS-945429, CNTO 136, CPSI-2364, CDP6038, VX30, ARGX-109, FE301, and FM101. In some embodiments, the anti-IL-6 antibody molecule is tocilizumab. In some embodiments, the additional agent is an IL-1R inhibitor, such as anakinra.

In some embodiments, the additional agent is a modulator of adenosine levels and/or an adenosine pathway component. Adenosine can function as an immunomodulatory agent in the body. For example, adenosine and some adenosine analogs that non-selectively activate adenosine receptor subtypes decrease neutrophil production of inflammatory oxidative products (Cronstein et al., Ann. N.Y. Acad. Sci. 451:291, 1985; Roberts et al., Biochem. J., 227:669, 1985; Schrier et al., J. Immunol. 137:3284, 1986; Cronstein et al., Clinical Immunol. Immunopath. 42:76, 1987). In some cases, concentration of extracellular adenosine or adenosine analogs can increase in specific environments, e.g., tumor microenvironment (TME). In some cases, adenosine or adenosine analog signaling depends on hypoxia or factors involved in hypoxia or its regulation, e.g., hypoxia inducible factor (HIF). In some embodiments, increase in adenosine signaling can increase in intracellular cAMP and cAMP-dependent protein kinase that results in inhibition of proinflammatory cytokine production, and can lead to the synthesis of immunosuppressive molecules and development of Tregs (Sitkovsky et al., Cancer Immunol Res (2014) 2(7):598-605). In some embodiments, the additional agent can reduce or reverse immunosuppressive effects of adenosine, adenosine analogs and/or adenosine signaling. In some embodiments, the additional agent can reduce or reverse hypoxia-driven A2-adenosinergic T cell immunosuppression. In some embodiments, the additional agent is selected from among antagonists of adenosine receptors, extracellular adenosine-degrading agents, inhibitors of adenosine generation by CD39/CD73 ectoenzymes, and inhibitors of hypoxia-HIF-la signaling. In some embodiments, the additional agent is an adenosine receptor antagonist or agonist.

Inhibition or reduction of extracellular adenosine or the adenosine receptor by virtue of an inhibitor of extracellular adenosine (such as an agent that prevents the formation of, degrades, renders inactive, and/or decreases extracellular adenosine), and/or an adenosine receptor inhibitor (such as an adenosine receptor antagonist) can enhance immune response, such as a macrophage, neutrophil, granulocyte, dendritic cell, T- and/or B cell-mediated response. In addition, inhibitors of the Gs protein mediated cAMP dependent intracellular pathway and inhibitors of the adenosine receptor-triggered Gi protein mediated intracellular pathways, can also increase acute and chronic inflammation.

In some embodiments, the additional agent is an adenosine receptor antagonist or agonist, e.g., an antagonist or agonist of one or more of the adenosine receptors A2a, A2b, A1, and A3. A1 and A3 inhibit, and A2a and A2b stimulate, respectively, adenylate cyclase activity. Certain adenosine receptors, such as A2a, A2b, and A3, can suppress or reduce the immune response during inflammation. Thus, antagonizing immunosuppressive adenosine receptors can augment, boost or enhance immune response, e.g., immune response from administered cells, e.g., CAR-expressing T cells. In some embodiments, the additional agent inhibits the production of extracellular adenosine and adenosine-triggered signaling through adenosine receptors. For example, enhancement of an immune response, local tissue inflammation, and targeted tissue destruction can be enhanced by inhibiting or reducing the adenosine-producing local tissue hypoxia; by degrading (or rendering inactive) accumulated extracellular adenosine; by preventing or decreasing expression of adenosine receptors on immune cells; and/or by inhibiting/antagonizing signaling by adenosine ligands through adenosine receptors.

An antagonist is any substance that tends to nullify the action of another, as an agent that binds to a cell receptor without eliciting a biological response. In some embodiments, the antagonist is a chemical compound that is an antagonist for an adenosine receptor, such as the A2a, A2b, or A3 receptor. In some embodiments, the antagonist is a peptide, or a peptidomimetic, that binds the adenosine receptor but does not trigger a Gi protein dependent intracellular pathway. Exemplary antagonists are described in U.S. Pat. Nos. 5,565,566; 5,545,627, 5,981,524; 5,861,405; 6,066,642; 6,326,390; 5,670,501; 6,117,998; 6,232,297; 5,786,360; 5,424,297; 6,313,131, 5,504,090; and 6,322,771.

In some embodiments, the additional agent is an A2 receptor (A2R) antagonist, such as an A2a antagonist. Exemplary A2R antagonists include KW6002 (istradefyline), SCH58261, caffeine, paraxanthine, 3,7-dimethyl-1-propargylxanthine (DMPX), 8-(m-chlorostyryl) caffeine (CSC), MSX-2, MSX-3, MSX-4, CGS-15943, ZM-241385, SCH-442416, preladenant, vipadenant (B11014), V2006, ST-1535, SYN-115, PSB-1115, ZM241365, FSPTP, and an inhibitory nucleic acid targeting A2R expression, e.g., siRNA or shRNA, or any antibodies or antigen-binding fragment thereof that targets an A2R. In some embodiments, the additional agent is an A2R antagonist described in, e.g., Ohta et al., Proc Natl Acad Sci USA (2006) 103:13132-13137; Jin et al., Cancer Res. (2010) 70(6):2245-2255; Leone et al., Computational and Structural Biotechnology Journal (2015) 13:265-272; Beavis et al., Proc Natl Acad Sci USA (2013) 110:14711-14716; and Pinna, A., Expert Opin Investig Drugs (2009) 18:1619-1631; Sitkovsky et al., Cancer Immunol Res (2014) 2(7):598-605; U.S. Pat. Nos. 8,080,554; 8,716,301; US 20140056922; WO2008/147482; U.S. Pat. No. 8,883,500; US 20140377240; WO02/055083; U.S. Pat. Nos. 7,141,575; 7,405,219; 8,883,500; 8,450,329 and 8,987,279).

In some embodiments, the antagonist is an antisense molecule, inhibitory nucleic acid molecule (e.g., small inhibitory RNA (siRNA)) or catalytic nucleic acid molecule (e.g. a ribozyme) that specifically binds mRNA encoding an adenosine receptor. In some embodiments, the antisense molecule, inhibitory nucleic acid molecule or catalytic nucleic acid molecule binds nucleic acids encoding A2a, A2b, or A3. In some embodiments, an antisense molecule, inhibitory nucleic acid molecule or catalytic nucleic acid targets biochemical pathways downstream of the adenosine receptor. For example, the antisense molecule or catalytic nucleic acid can inhibit an enzyme involved in the Gs protein- or Gi protein-dependent intracellular pathway. In some embodiments, the additional agent includes dominant negative mutant form of an adenosine receptor, such as A2a, A2b, or A3.

In some embodiments, the additional agent that inhibits extracellular adenosine includes agents that render extracellular adenosine non-functional (or decrease such function), such as a substance that modifies the structure of adenosine to inhibit the ability of adenosine to signal through adenosine receptors. In some embodiments, the additional agent is an extracellular adenosine-generating or adenosine-degrading enzyme, a modified form thereof or a modulator thereof. For example, in some embodiments, the additional agent is an enzyme (e.g. adenosine deaminase) or another catalytic molecule that selectively binds and destroys the adenosine, thereby abolishing or significantly decreasing the ability of endogenously formed adenosine to signal through adenosine receptors and terminate inflammation.

In some embodiments, the additional agent is an adenosine deaminase (ADA) or a modified form thereof, e.g., recombinant ADA and/or polyethylene glycol-modified ADA (ADA-PEG), which can inhibit local tissue accumulation of extracellular adenosine. ADA-PEG has been used in treatment of patients with ADA SCID (Hershfield (1995) Hum Mutat. 5:107). In some embodiments, an agent that inhibits extracellular adenosine includes agents that prevent or decrease formation of extracellular adenosine, and/or prevent or decrease the accumulation of extracellular adenosine, thereby abolishing, or substantially decreasing, the immunosuppressive effects of adenosine. In some embodiments, the additional agent specifically inhibits enzymes and proteins that are involved in regulation of synthesis and/or secretion of pro-inflammatory molecules, including modulators of nuclear transcription factors. Suppression of adenosine receptor expression or expression of the Gs protein- or Gi protein-dependent intracellular pathway, or the cAMP dependent intracellular pathway, can result in an increase/enhancement of immune response.

In some embodiments, the additional agent can target ectoenzymes that generate or produce extracellular adenosine. In some embodiments, the additional agent targets CD39 and CD73 ectoenzymes, which function in tandem to generate extracellular adenosine. CD39 (also called ectonucleoside triphosphate diphosphohydrolase) converts extracellular ATP (or ADP) to 5′AMP. Subsequently, CD73 (also called 5′nucleotidase) converts 5′AMP to adenosine. The activity of CD39 is reversible by the actions of NDP kinase and adenylate kinase, whereas the activity of CD73 is irreversible. CD39 and CD73 are expressed on tumor stromal cells, including endothelial cells and Tregs, and also on many cancer cells. For example, the expression of CD39 and CD73 on endothelial cells is increased under the hypoxic conditions of the tumor microenvironment. Tumor hypoxia can result from inadequate blood supply and disorganized tumor vasculature, impairing delivery of oxygen (Carroll and Ashcroft (2005), Expert. Rev. Mol. Med. 7(6):1-16). Hypoxia also inhibits adenylate kinase (AK), which converts adenosine to AMP, leading to very high extracellular adenosine concentration. Thus, adenosine is released at high concentrations in response to hypoxia, which is a condition that frequently occurs the tumor microenvironment (TME), in or around solid tumors. In some embodiments, the additional agent is one or more of anti-CD39 antibody or antigen binding fragment thereof, anti-CD73 antibody or antigen binding fragment thereof, e.g., MEDI9447 or TY/23, α-β-methylene-adenosine diphosphate (ADP), ARL 67156, POM-3, IPH52 (see, e.g., Allard et al. Clin Cancer Res (2013) 19(20):5626-5635; Hausler et al., Am J Transl Res (2014) 6(2):129-139; Zhang, B., Cancer Res. (2010) 70(16):6407-6411).

In some embodiments, the additional agent is an inhibitor of hypoxia inducible factor 1 alpha (HIF-la) signaling. Exemplary inhibitors of HIF-la include digoxin, acriflavine, sirtuin-7 and ganetespib.

In some embodiments, the additional agent includes a protein tyrosine phosphatase inhibitor, e.g., a protein tyrosine phosphatase inhibitor described herein. In some embodiments, the protein tyrosine phosphatase inhibitor is an SHP-1 inhibitor, e.g., an SHP-1 inhibitor described herein, such as, e.g., sodium stibogluconate. In some embodiments, the protein tyrosine phosphatase inhibitor is an SHP-2 inhibitor, e.g., an SHP-2 inhibitor described herein.

In some embodiments, the additional agent is a kinase inhibitor. Kinase inhibitors, such as a CDK4 kinase inhibitor, a BTK kinase inhibitor, a MNK kinase inhibitor, or a DGK kinase inhibitor, can regulate the constitutively active survival pathways that exist in tumor cells and/or modulate the function of immune cells. In some embodiments, the kinase inhibitor is a Bruton's tyrosine kinase (BTK) inhibitor, e.g., ibrutinib. In some embodiments, the kinase inhibitor is a phosphatidylinositol-4,5-bisphosphate 3-kinase (PI3K) inhibitor. In some embodiments, the kinase inhibitor is a CDK4 inhibitor, e.g., a CDK4/6 inhibitor. In some embodiments, the kinase inhibitor is an mTOR inhibitor, such as, e.g., rapamycin, a rapamycin analog, OSI-027. The mTOR inhibitor can be, e.g., an mTORC1 inhibitor and/or an mTORC2 inhibitor, e.g., an mTORC1 inhibitor and/or mTORC2 inhibitor. In some embodiments, the kinase inhibitor is an MNK inhibitor, or a dual PI3K/mTOR inhibitor. In some embodiments, other exemplary kinase inhibitors include the AKT inhibitor perifosine, the mTOR inhibitor temsirolimus, the Src kinase inhibitors dasatinib and fostamatinib, the JAK2 inhibitors pacritinib and ruxolitinib, the PKCP inhibitors enzastaurin and bryostatin, and the AAK inhibitor alisertib.

In some embodiments, the kinase inhibitor is a BTK inhibitor selected from ibrutinib (PCI-32765); GDC-0834; RN-486; CGI-560; CGI-1764; HM-71224; CC-292; ONO-4059; CNX-774; and LFM-A13. In some embodiments, the BTK inhibitor does not reduce or inhibit the kinase activity of interleukin-2-inducible kinase (ITK), and is selected from GDC-0834; RN-486; CGI-560; CGI-1764; HM-71224; CC-292; ONO-4059; CNX-774; and LFM-A13.

In some embodiments, the kinase inhibitor is a BTK inhibitor, e.g., ibrutinib (1-[(3R)-3-[4-Amino-3-(4-phenoxyphenyl)-1H-pyrazolo[3,4-d]pyrimidin-1-yl]piperidin-1-yl]prop-2-en-1-one; also known as PCI-32765). In some embodiments, the kinase inhibitor is a BTK inhibitor, e.g., ibrutinib (PCI-32765). In some embodiments, 1, 2, 3, 4, 5, 6, 7, 8, 9, 10, 11, 12 or more cycles of ibrutinib are administered. In some embodiments, the BTK inhibitor is a BTK inhibitor described in International Application WO 2015/079417.

In some embodiments, the kinase inhibitor is a PI3K inhibitor. PI3K is central to the PI3K/Akt/mTOR pathway involved in cell cycle regulation and lymphoma survival. Exemplary PI3K inhibitor includes idelalisib (PI3K6 inhibitor). In some embodiments, the additional agent is idelalisib and rituximab.

In some embodiments, the additional agent is an inhibitor of mammalian target of rapamycin (mTOR). In some embodiments, the kinase inhibitor is an mTOR inhibitor selected from temsirolimus; ridaforolimus (also known as AP23573 and MK8669); everolimus (RAD001); rapamycin (AY22989); simapimod; AZD8055; PF04691502; SF1126; and XL765. In some embodiments, the additional agent is an inhibitor of mitogen-activated protein kinase (MAPK), such as vemurafenib, dabrafenib, and trametinib.

In some embodiments, the additional agent is an agent that regulates pro- or anti-apoptotic proteins. In some embodiments, the additional agent includes a B-cell lymphoma 2 (BCL-2) inhibitor (e.g., venetoclax, also called ABT-199 or GDC-0199; or ABT-737). Venetoclax is a small molecule (4-(4-{[2-(4-Chlorophenyl)-4,4-dimethyl-1-cyclohexen-1-yl]methyl}-1-piperazinyl)-N-({3-nitro-4-[(tetrahydro-2H-pyran-4-ylmethyl)amino]phenyl}sulfonyl)-2-(1H-pyrrolo[2,3-b]pyridin-5-yloxy)benzamide) that inhibits the anti-apoptotic protein, BCL-2. Other agents that modulate pro- or anti-apoptotic protein include BCL-2 inhibitor ABT-737, navitoclax (ABT-263); Mcl-1 siRNA or Mcl-1 inhibitor retinoid N-(4-hydroxyphenyl) retinamide (4-HPR) for maximal efficacy. In some embodiments, the additional agent provides a pro-apoptotic stimuli, such as recombinant tumor necrosis factor-related apoptosis-inducing ligand (TRAIL), which can activate the apoptosis pathway by binding to TRAIL death receptors DR-4 and DR-5 on tumor cell surface, or TRAIL-R2 agonistic antibodies.

In some embodiments, the additional agent includes an indoleamine 2,3-dioxygenase (IDO) inhibitor. IDO is an enzyme that catalyzes the degradation of the amino acid, L-tryptophan, to kynurenine. Many cancers overexpress IDO, e.g., prostatic, colorectal, pancreatic, cervical, gastric, ovarian, head, and lung cancer. Plasmacytoid dendritic cells (pDCs), macrophages, and dendritic cells (DCs) can express IDO. In some aspects, a decrease in L-tryptophan (e.g., catalyzed by IDO) results in an immunosuppressive milieu by inducing T-cell anergy and apoptosis. Thus, in some aspects, an IDO inhibitor can enhance the efficacy of the binding molecules (e.g., ROR1-binding molecules), recombinant receptors, cells and/or compositions described herein, e.g., by decreasing the suppression or death of the administered CAR-expressing cell. Exemplary inhibitors of IDO include but are not limited to 1-methyl-tryptophan, indoximod, and INCB024360 (epacadostat).

In some embodiments, the additional agent includes a cytotoxic agent, e.g., CPX-351 (Celator Pharmaceuticals), cytarabine, daunorubicin, vosaroxin (Sunesis Pharmaceuticals), sapacitabine (Cyclacel Pharmaceuticals), idarubicin, or mitoxantrone. In some embodiments, the additional agent includes a hypomethylating agent, e.g., a DNA methyltransferase inhibitor, e.g., azacitidine or decitabine.

In another embodiment, the additional therapy is a transplantation, e.g., allogeneic stem cell transplant.

In some embodiments, the additional therapy is a lymphodepleting therapy. In some embodiments, lymphodepletion is performed on a subject, e.g., prior to administering engineered cells, e.g., CAR-expressing cells. In some embodiments, the lymphodepletion comprises administering one or more of melphalan, Cytoxan, cyclophosphamide, and fludarabine. In some embodiments, a lymphodepleting chemotherapy is administered to the subject prior to, concurrently with, or after administration (e.g., infusion) of engineered cells, e.g., CAR-expressing cells. In an example, the lymphodepleting chemotherapy is administered to the subject prior to administration of engineered cells, e.g., CAR-expressing cells.

In some embodiments, the additional agent is an oncolytic virus. In some embodiments, oncolytic viruses are capable of selectively replicating in and triggering the death of or slowing the growth of a cancer cell. In some cases, oncolytic viruses have no effect or a minimal effect on non-cancer cells. An oncolytic virus includes but is not limited to an oncolytic adenovirus, oncolytic Herpes Simplex Viruses, oncolytic retrovirus, oncolytic parvovirus, oncolytic vaccinia virus, oncolytic Sinbis virus, oncolytic influenza virus, or oncolytic RNA virus (e.g., oncolytic reovirus, oncolytic Newcastle Disease Virus (NDV), oncolytic measles virus, or oncolytic vesicular stomatitis virus (VSV)).

Other exemplary combination therapy, treatment and/or agents include anti-allergenic agents, anti-emetics, analgesics and adjunct therapies. In some embodiments, the additional agent includes cytoprotective agents, such as neuroprotectants, free-radical scavengers, cardioprotectors, anthracycline extravasation neutralizers and nutrients.

In some embodiments, an antibody used as an additional agent is conjugated or otherwise bound to a therapeutic agent, e.g., a chemotherapeutic agent (e.g., Cytoxan, fludarabine, histone deacetylase inhibitor, demethylating agent, peptide vaccine, anti-tumor antibiotic, tyrosine kinase inhibitor, alkylating agent, anti-microtubule or anti-mitotic agent), anti-allergic agent, anti-nausea agent (or anti-emetic), pain reliever, or cytoprotective agent described herein. In some embodiments, the additional agent is an antibody-drug conjugate.

In some embodiments, the additional agent can modulate, inhibit or stimulate particular factors at the DNA, RNA or protein levels, to enhance or boost the efficacy of the binding molecules (e.g., ROR1-binding molecules), recombinant receptors, cells and/or compositions provided herein. In some embodiments, the additional agent can modulate the factors at the nucleic acid level, e.g., DNA or RNA, within the administered cells, e.g., cells engineered to express recombinant receptors, e.g., CAR. In some embodiments, an inhibitory nucleic acid, e.g., an inhibitory nucleic acid, e.g., a dsRNA, e.g., an siRNA or shRNA, or a clustered regularly interspaced short palindromic repeats (CRISPR), a transcription-activator like effector nuclease (TALEN), or a zinc finger endonuclease (ZFN), can be used to inhibit expression of an inhibitory molecule in the engineered cell, e.g., CAR-expressing cell. In some embodiments the inhibitor is an shRNA. In some embodiments, the inhibitory molecule is inhibited within the engineered cell, e.g., CAR-expressing cell. In some embodiments, a nucleic acid molecule that encodes a dsRNA molecule that inhibits expression of the molecule that modulates or regulates, e.g., inhibits, T-cell function is operably linked to a promoter, e.g., a HI- or a U6-derived promoter such that the dsRNA molecule that inhibits expression of the inhibitory molecule is expressed within the engineered cell, e.g., CAR-expressing cell. See, e.g., Brummelkamp T R, et al. (2002) Science 296: 550-553; Miyagishi M, et al. (2002) Nat. Biotechnol. 19: 497-500.

In some embodiments, the additional agent is capable of disrupting the gene encoding an inhibitory molecule, such as any immune checkpoint inhibitors described herein. In some embodiments, disruption is by deletion, e.g., deletion of an entire gene, exon, or region, and/or replacement with an exogenous sequence, and/or by mutation, e.g., frameshift or missense mutation, within the gene, typically within an exon of the gene. In some embodiments, the disruption results in a premature stop codon being incorporated into the gene, such that the inhibitory molecule is not expressed or is not expressed in a form that is capable of being expressed on the cells surface and/or capable of mediating cell signaling. The disruption is generally carried out at the DNA level. The disruption generally is permanent, irreversible, or not transient.

In some aspects, the disruption is carried out by gene editing, such as using a DNA binding protein or DNA-binding nucleic acid, which specifically binds to or hybridizes to the gene at a region targeted for disruption. In some aspects, the protein or nucleic acid is coupled to or complexed with a nuclease, such as in a chimeric or fusion protein. For example, in some embodiments, the disruption is effected using a fusion comprising a DNA-targeting protein and a nuclease, such as a Zinc Finger Nuclease (ZFN) or TAL-effector nuclease (TALEN), or an RNA-guided nuclease such as a clustered regularly interspersed short palindromic nucleic acid (CRISPR)-Cas system, such as CRISPR-Cas9 system, specific for the gene being disrupted. In some embodiments, methods of producing or generating genetically engineered cells, e.g., CAR-expressing cells, include introducing into a population of cells nucleic acid molecules encoding a genetically engineered antigen receptor (e.g. CAR) and nucleic acid molecules encoding an agent targeting an inhibitory molecule that is a gene editing nuclease, such as a fusion of a DNA-targeting protein and a nuclease such as a ZFN or a TALEN, or an RNA-guided nuclease such as of the CRISPR-Cas9 system, specific for an inhibitory molecule.

Any of the additional agents described herein can be prepared and administered as combination therapy with the ROR1-binding molecule (e.g., antibody), immunoconjugate, recombinant receptor (e.g., chimeric antigen receptor) and/or engineered cells expressing said molecules (e.g., recombinant receptor) described herein, such as in pharmaceutical compositions comprising one or more agents of the combination therapy and a pharmaceutically acceptable carrier, such as any described herein. In some embodiments, the ROR1-binding molecule (e.g., antibody), immunoconjugate, recombinant receptor (e.g., chimeric antigen receptor), engineered cells expressing said molecules (e.g., recombinant receptor), plurality of engineered cells expressing said molecules (e.g., recombinant receptor) can be administered simultaneously, concurrently or sequentially, in any order with the additional agents, therapy or treatment, wherein such administration provides therapeutically effective levels each of the agents in the body of the subject. The agents can be co-administered with the binding molecules (e.g., ROR1-binding molecules), recombinant receptors, cells and/or compositions described herein, for example, as part of the same pharmaceutical composition or using the same method of delivery. In some embodiments, the additional agent is incubated with the engineered cell, e.g., CAR-expressing cells, prior to administration of the cells.

In some examples, the one or more additional agents are administered subsequent to or prior to the administration of the binding molecules (e.g., ROR1-binding molecules), recombinant receptors, cells and/or compositions described herein, separated by a selected time period. In some examples, the time period is 1 day, 2 days, 3 days, 4 days, 5 days, 6 days, 1 week, 2 weeks, 3 weeks, 1 month, 2 months, or 3 months. In some examples, the one or more additional agents are administered multiple times and/or the binding molecules (e.g., ROR1-binding molecules), recombinant receptors, cells and/or compositions described herein, is administered multiple times. For example, in some embodiments, the additional agent is administered prior to the binding molecules (e.g., ROR1-binding molecules), recombinant receptors, cells and/or compositions described herein, e.g., two weeks, 12 days, 10 days, 8 days, one week, 6 days, 5 days, 4 days, 3 days, 2 days or 1 day before the administration. For example, in some embodiments, the additional agent is administered after the binding molecules (e.g., ROR1-binding molecules), recombinant receptors, cells and/or compositions described herein, e.g., two weeks, 12 days, 10 days, 8 days, one week, 6 days, 5 days, 4 days, 3 days, 2 days or 1 day after the administration.

The dose of the additional agent can be any therapeutically effective amount, e.g., any dose amount described herein, and the appropriate dosage of the additional agent may depend on the type of disease to be treated, the type, dose and/or frequency of the binding molecule, recombinant receptor, cell and/or composition administered, the severity and course of the disease, whether the binding molecule, recombinant receptor, cell and/or composition is administered for preventive or therapeutic purposes, previous therapy, the patient's clinical history and response to the binding molecule, recombinant receptor, cell and/or composition, and the discretion of the attending physician. The binding molecule, recombinant receptor, cell and/or composition and/or the additional agent and/or therapy can be administered to the patient at one time, repeated or administered over a series of treatments.

### C. Diagnostic and Detection Methods

Also provided are methods involving use of the provided binding molecules, e.g., antibodies or antigen-binding fragments thereof, in detection of ROR1, for example, in diagnostic and/or prognostic methods in association with a ROR1-expressing disease or condition. The methods in some embodiments include incubating a biological sample with the antibody or antigen-binding fragment thereof and/or administering the antibody or antigen-binding fragment thereof to a subject. In certain embodiments, a biological sample includes a cell or tissue, such as tumor or cancer tissue. In certain embodiments, the contacting is under conditions permissive for binding of the anti-ROR1 antibody to ROR1, and detecting whether a complex is formed between the anti-ROR1 antibody and ROR1. Such a method may be an in vitro or in vivo method. In one embodiment, an anti-ROR1 antibody (e.g., antigen-binding fragment) is used to select subjects eligible for therapy with an anti-ROR1 antibody (e.g., antigen-binding fragment) or recombinant receptor, e.g. where ROR1 is a biomarker for selection of patients.

In some embodiments, a sample, such as a cell, tissue sample, lysate, composition, or other sample derived therefrom is contacted with the anti-ROR1 antibody (e.g., antigen-binding fragment) and binding or formation of a complex between the antibody and the sample (e.g., ROR1 in the sample) is determined or detected. When binding in the test sample is demonstrated or detected as compared to a reference cell of the same tissue type, it may indicate the presence of an associated disease or condition. In some embodiments, the sample is from human tissues.

Various known methods for detecting specific antibody-antigen binding can be used. Exemplary immunoassays include fluorescence polarization immunoassay (FPIA), fluorescence immunoassay (FIA), enzyme immunoassay (EIA), nephelometric inhibition immunoassay (NIA), enzyme linked immunosorbent assay (ELISA), and radioimmunoassay (RIA). An indicator moiety, or label group, can be attached to the subject antibodies and is selected so as to meet the needs of various uses of the method which are often dictated by the availability of assay equipment and compatible immunoassay procedures. Exemplary labels include radionuclides (e.g. ¹⁵I, ¹³¹I, ³⁵S, ³H, or ³²P), enzymes (e.g., alkaline phosphatase, horseradish peroxidase, luciferase, or β-galactosidase), fluorescent moieties or proteins (e.g., fluorescein, rhodamine, phycoerythrin, GFP, or BFP), or luminescent moieties (e.g., Qdot™ nanoparticles supplied by the Quantum Dot Corporation, Palo Alto, Calif.). General techniques to be used in performing the various immunoassays noted above are known to those of ordinary skill in the art.

For purposes of diagnosis, the antibodies (e.g., antigen-binding fragments) can be labeled with a detectable moiety including but not limited to radioisotopes, fluorescent labels, and various enzyme-substrate labels know in the art. Methods of conjugating labels to an antibody are known.

In some embodiments, antibodies (e.g., antigen-binding fragments) need not be labeled, and the presence thereof can be detected using a labeled antibody which binds to the antibodies.

The provided antibodies (e.g., antigen-binding fragments) in some embodiments can be employed in any known assay method, such as competitive binding assays, direct and indirect sandwich assays, and immunoprecipitation assays. Zola, Monoclonal Antibodies: A Manual of Techniques, pp. 147-158 (CRC Press, Inc. 1987).

The antibodies (e.g., antigen-binding fragments) and polypeptides can also be used for in vivo diagnostic assays, such as in vivo imaging. Generally, the antibody is labeled with a radionuclide (such as ¹¹¹In, ⁹⁹Tc, ¹⁴C, ¹³¹I, ¹²⁵I, or ³H) so that the cells or tissue of interest can be localized in vivo following administration to a subject.

The antibody (e.g., antigen-binding fragment) may also be used as staining reagent in pathology, e.g., using known techniques.

### V. ARTICLES OF MANUFACTURE OR KITS

Also provided are articles of manufacture or kit containing the provided binding molecules (e.g., antibodies), recombinant receptors (e.g., CARs), genetically engineered cells, and/or compositions comprising the same. The articles of manufacture may include a container and a label or package insert on or associated with the container. Suitable containers include, for example, bottles, vials, syringes, test tubes, IV solution bags, etc. The containers may be formed from a variety of materials such as glass or plastic. In some embodiments, the container has a sterile access port. Exemplary containers include an intravenous solution bags, vials, including those with stoppers pierceable by a needle for injection. The article of manufacture or kit may further include a package insert indicating that the compositions can be used to treat a particular condition such as a condition described herein (e.g., ROR1-expressing cancer). Alternatively, or additionally, the article of manufacture or kit may further include another or the same container comprising a pharmaceutically-acceptable buffer. It may further include other materials such as other buffers, diluents, filters, needles, and/or syringes.

The label or package insert may indicate that the composition is used for treating the ROR1-expressing or ROR1-associated disease, disorder or condition in an individual. The label or a package insert, which is on or associated with the container, may indicate directions for reconstitution and/or use of the formulation. The label or package insert may further indicate that the formulation is useful or intended for subcutaneous, intravenous, or other modes of administration for treating or preventing a ROR1-expressing or ROR1-associated disease, disorder or condition in an individual. In some aspects, the label or package insert can include instructions for use, for example instructions for administering the antibody or antigen-binding fragment thereof, the single chain cell surface protein, the conjugate, the chimeric antigen receptor, the cell or the composition, in some aspects in accord with any of the methods or uses described herein.

The container in some embodiments holds a composition which is by itself or combined with another composition effective for treating, preventing and/or diagnosing the condition. The article of manufacture or kit may include (a) a first container with a composition contained therein (i.e., first medicament), wherein the composition includes the antibody (e.g., anti-ROR1 antibody) or antigen-binding fragment thereof or recombinant receptor (e.g., CAR); and (b) a second container with a composition contained therein (i.e., second medicament), wherein the composition includes a further agent, such as a cytotoxic or otherwise therapeutic agent, and which article or kit further comprises instructions on the label or package insert for treating the subject with the second medicament, in an effective amount.

### VI. Definitions

As used herein, reference to a “corresponding form” of an antibody means that when comparing a property or activity of two antibodies, the property is compared using the same form of the antibody. For example, if it is stated that an antibody has greater activity compared to the activity of the corresponding form of a first antibody, that means that a particular form, such as an scFv of that antibody, has greater activity compared to the scFv form of the first antibody.

“Effector functions” refer to those biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g. B cell receptor); and B cell activation.

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991.

The terms “full length antibody,” “intact antibody,” and “whole antibody” are used herein interchangeably to refer to an antibody having a structure substantially similar to a native antibody structure or having heavy chains that contain an Fc region as defined herein.

An “isolated” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr. B 848:79-87 (2007).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding an anti-ROR1 antibody” refers to one or more nucleic acid molecules encoding antibody heavy and light chains (or fragments thereof), including such nucleic acid molecule(s) in a single vector or separate vectors, and such nucleic acid molecule(s) present at one or more locations in a host cell.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

As used herein, “percent (%) amino acid sequence identity” and “percent identity” and “sequence identity” when used with respect to an amino acid sequence (reference polypeptide sequence) is defined as the percentage of amino acid residues in a candidate sequence (e.g., the subject antibody or fragment) that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

An amino acid substitution may include replacement of one amino acid in a polypeptide with another amino acid. The substitution may be a conservative amino acid substitution or a non-conservative amino acid substitution. Amino acid substitutions may be introduced into a binding molecule, e.g., antibody, of interest and the products screened for a desired activity, e.g., retained/improved antigen binding, decreased immunogenicity, or improved ADCC or CDC.

Amino acids generally can be grouped according to the following common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

In some embodiments, conservative substitutions can involve the exchange of a member of one of these classes for another member of the same class. In some embodiments, non-conservative amino acid substitutions can involve exchanging a member of one of these classes for another class.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The term “package insert” is used to refer to instructions customarily included in commercial packages of therapeutic products, that contain information about the indications, usage, dosage, administration, combination therapy, contraindications and/or warnings concerning the use of such therapeutic products.

As used herein, the singular forms “a,” “an,” and “the” include plural referents unless the context clearly dictates otherwise. For example, “a” or “an” means “at least one” or “one or more.” It is understood that aspects, embodiments, and variations described herein include “comprising,” “consisting,” and/or “consisting essentially of” aspects, embodiments and variations.

Throughout this disclosure, various aspects of the claimed subject matter are presented in a range format. It should be understood that the description in range format is merely for convenience and brevity and should not be construed as an inflexible limitation on the scope of the claimed subject matter.

Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X”.

As used herein, a “composition” refers to any mixture of two or more products, substances, or compounds, including cells. It may be a solution, a suspension, liquid, powder, a paste, aqueous, non-aqueous or any combination thereof.

As used herein, a statement that a cell or population of cells is “positive” for a particular marker refers to the detectable presence on or in the cell of a particular marker, typically a surface marker. When referring to a surface marker, the term refers to the presence of surface expression as detected by flow cytometry, for example, by staining with an antibody that specifically binds to the marker and detecting said antibody, wherein the staining is detectable by flow cytometry at a level substantially above the staining detected carrying out the same procedure with an isotype-matched control under otherwise identical conditions and/or at a level substantially similar to that for cell known to be positive for the marker, and/or at a level substantially higher than that for a cell known to be negative for the marker.

As used herein, a statement that a cell or population of cells is “negative” for a particular marker refers to the absence of substantial detectable presence on or in the cell of a particular marker, typically a surface marker. When referring to a surface marker, the term refers to the absence of surface expression as detected by flow cytometry, for example, by staining with an antibody that specifically binds to the marker and detecting said antibody, wherein the staining is not detected by flow cytometry at a level substantially above the staining detected carrying out the same procedure with an isotype-matched control under otherwise identical conditions, and/or at a level substantially lower than that for cell known to be positive for the marker, and/or at a level substantially similar as compared to that for a cell known to be negative for the marker.

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

### VII. Exemplary Embodiments

Among the provided embodiments are:

1. An anti-ROR1 antibody or antigen-binding fragment thereof, comprising: a heavy chain variable (V_{H}) region, and a light chain variable (V_{L}) region, wherein the V_{H} region comprises a heavy chain complementarity determining region 1 (CDR-H1) comprising the sequence set forth in SEQ ID NO: 67, 82 or 52, a heavy chain complementarity determining region 2 (CDR-H2) comprising the sequence set forth in SEQ ID NO: 71, 86, 56 or 97, and a heavy chain complementarity determining region 3 (CDR-H3) comprising the sequence set forth in SEQ ID NO: 73, 88, 58 or 99, and the V_{L} region comprises a light chain complementarity determining region 1 (CDR-L1) comprising the sequence set forth in SEQ ID NO: 75, 90 or 60, a light chain complementarity determining region 2 (CDR-L2) comprising the sequence set forth in SEQ ID NO: 77, 92 or 62; and a light chain complementarity determining region 3 (CDR-L3) comprising the sequence set forth in SEQ ID NO: 79, 94 or 64.

2. An anti-ROR1 antibody or antigen-binding fragment thereof, comprising: a heavy chain variable (V_{H}) region, and a light chain variable (V_{L}) region, wherein the V_{H} region comprises a heavy chain complementarity determining region 1 (CDR-H1), a heavy chain complementarity determining region 2 (CDR-H2) and a heavy chain complementarity determining region 3 (CDR-H3) comprising the sequence set forth in SEQ ID NOS:67, 71 and 73, respectively, and the V_{L} region comprises a light chain complementarity determining region 1 (CDR-L1), a light chain complementarity determining region 2 (CDR-L2) and a light chain complementarity determining region 3 (CDR-L3) comprising the sequence set forth in SEQ ID NOS:75, 77 and 79, respectively;
the V_{H} region comprises a CDR-H1, a CDR-H2 and a CDR-H3 comprising the sequence set forth in SEQ ID NOS:82, 86 and 88, respectively, and the V_{L} region comprises a CDR-L1, a CDR-L2 and a CDR-L3 comprising the sequence set forth in SEQ ID NOS:90, 92 and 94, respectively;
the V_{H} region comprises a CDR-H1, a CDR-H2 and a CDR-H3 comprising the sequence set forth in SEQ ID NOS:52, 56 and 58, respectively, and the V_{L} region comprises a CDR-L1, a CDR-L2 and a CDR-L3 comprising the sequence set forth in SEQ ID NOS:60, 62 and 64, respectively; or
the V_{H} region comprises a CDR-H1, a CDR-H2 and a CDR-H3 comprising the sequence set forth in SEQ ID NOS:52, 97 and 99, respectively, and the V_{L} region comprises a CDR-L1, a CDR-L2 and a CDR-L3 comprising the sequence set forth in SEQ ID NOS:60, 62 and 64, respectively.

3. The anti-ROR1 antibody or antigen-binding fragment thereof of embodiment 1 or embodiment 2, wherein the V_{H} region comprises a CDR-H1, a CDR-H2 and a CDR-H3 comprising the sequence set forth in SEQ ID NOS:67, 71 and 73, respectively, and the V_{L} region comprises a CDR-L1, a CDR-L2 and a CDR-L3 comprising the sequence set forth in SEQ ID NOS:75, 77 and 79, respectively.

4. The anti-ROR1 antibody or antigen-binding fragment thereof of embodiment 1 or embodiment 2, wherein the V_{H} region comprises a CDR-H1, a CDR-H2 and a CDR-H3 comprising the sequence set forth in SEQ ID NOS:82, 86 and 88, respectively, and the V_{L} region comprises a CDR-L1, a CDR-L2 and a CDR-L3 comprising the sequence set forth in SEQ ID NOS:90, 92 and 94, respectively.

5. An anti-ROR1 antibody or antigen-binding fragment thereof, comprising: a heavy chain variable (V_{H}) region and a light chain variable (V_{L}) region, wherein:
the V_{H} region comprises a heavy chain complementarity determining region 1 (CDR-H1), a heavy chain complementarity determining region 2 (CDR-H2) and a heavy chain complementarity determining region 3 (CDR-H3) contained within SEQ ID NO: 112, 121, 103 or 130, and
the V_{L} region comprises a light chain complementarity determining region 1 (CDR-L1), a light chain complementarity determining region 2 (CDR-L2) and a light chain complementarity determining region 3 (CDR-L3) contained within SEQ ID NO: 115, 124 or 106.

6. An anti-ROR1 antibody or antigen-binding fragment thereof, comprising: a heavy chain variable (V_{H}) region and a light chain variable (V_{L}) region, wherein:
the V_{H} region comprises a heavy chain complementarity determining region 1 (CDR-H1), a heavy chain complementarity determining region 2 (CDR-H2) and a heavy chain complementarity determining region 3 (CDR-H3) contained within SEQ ID NO:112, and the V_{L} region comprises a light chain complementarity determining region 1 (CDR-L1), a light chain complementarity determining region 2 (CDR-L2) and a light chain complementarity determining region 3 (CDR-L3) contained within SEQ ID NO:115;
the V_{H} region comprises a CDR-H1, a CDR-H2 and a CDR-H3 contained within SEQ ID NO:121, and the V_{L} region comprises a CDR-L1, a CDR-L2 and a CDR-L3 contained within SEQ ID: NO 124;
the V_{H} region comprises a CDR-H1, a CDR-H2 and a CDR-H3 contained within SEQ ID NO:103, and the V_{L} region comprises a CDR-L1, a CDR-L2 and a CDR-L3 contained within SEQ ID: NO 106; or
the V_{H} region comprises a CDR-H1, a CDR-H2 and a CDR-H3 contained within SEQ ID NO:130, and the V_{L} region comprises a CDR-L1, a CDR-L2 and a CDR-L3 contained within SEQ ID: NO 106.

7. The anti-ROR1 antibody or antigen-binding fragment thereof of embodiment 5 or embodiment 6, wherein the V_{H} region comprises a CDR-H1, a CDR-H2 and a CDR-H3 contained within SEQ ID NO:112, and the V_{L} region comprises a CDR-L1, a CDR-L2 and a CDR-L3 contained within SEQ ID: NO 115.

8. The anti-ROR1 antibody or antigen-binding fragment thereof of embodiment 5 or embodiment 6, wherein the V_{H} region comprises a CDR-H1, a CDR-H2 and a CDR-H3 contained within SEQ ID NO:121, and the V_{L} region comprises a CDR-L1, a CDR-L2 and a CDR-L3 contained within SEQ ID: NO 124.

9. The anti-ROR1 antibody or antigen-binding fragment thereof of any of embodiments 1, 2, 5 and 6, wherein:
the V_{H} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 112, 121, 103 or 130, and
the V_{L} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 115, 124 or 106.

10. The anti-ROR1 antibody or antigen-binding fragment thereof of any of embodiments 1, 2, and 5-7, wherein:
the V_{H} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO:112, and the V_{L} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 115;
the V_{H} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO:121, and the V_{L} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO:124;
the V_{H} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO:103, and the V_{L} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO:106; or
the V_{H} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO:130, and the V_{L} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO:106.

11. The anti-ROR1 antibody or antigen-binding fragment thereof of any of embodiments 1-10, wherein the V_{H} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO:112, and the V_{L} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 115.

12. The anti-ROR1 antibody or antigen-binding fragment thereof of any of embodiments 1-10, wherein the V_{H} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO:121, and the V_{L} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO:124.

13. An anti-ROR1 antibody or antigen-binding fragment thereof, comprising: a heavy chain variable (V_{H}) region and a light chain variable (V_{L}) region, wherein:
the V_{H} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 112, 121, 103 or 130, and
the V_{L} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 115, 124 or 106.

14. An anti-ROR1 antibody or antigen-binding fragment thereof, comprising: a heavy chain variable (V_{H}) region and a light chain variable (V_{L}) region, wherein:
the V_{H} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO:112, and the V_{L} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 115;
the V_{H} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO:121, and the V_{L} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO:124;
the V_{H} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO:103, and the V_{L} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO:106; or
the V_{H} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO:130, and the V_{L} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO:106.

15. The anti-ROR1 antibody or antigen-binding fragment thereof of embodiment 13 or embodiment 14, wherein the V_{H} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO:112, and the V_{L} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 115.

16. The anti-ROR1 antibody or antigen-binding fragment thereof of embodiment 13 or embodiment 14, wherein the V_{H} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO:121, and the V_{L} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO:124.

17. The anti-ROR1 antibody or antigen-binding fragment thereof of any of embodiments 1, 2, 5, 6, 9, 10, 13 and 14, wherein:
the V_{H} region is or comprises the sequence set forth in SEQ ID NO: 112, 121, 103 or 130, and
the V_{L} region is or comprises the sequence set forth in SEQ ID NO: 115, 124 or 106.

18. The anti-ROR1 antibody or antigen-binding fragment thereof of any of embodiments 1, 2, 5, 6, 9, 10, 13, 14 and 17, wherein:
the V_{H} region and the V_{L} region are or comprise the sequence set forth in SEQ ID NOS: 112 and 115, respectively;
the V_{H} region and the V_{L} region are or comprise the sequence set forth in SEQ ID NOS: 121 and 124, respectively;
the V_{H} region and the V_{L} region are or comprise the sequence set forth in SEQ ID NOS: 103 and 106, respectively; or
the V_{H} region and the V_{L} region are or comprise the sequence set forth in SEQ ID NOS: 130 and 106, respectively.

19. An anti-ROR1 antibody or antigen-binding fragment thereof, comprising: a heavy chain variable (V_{H}) region and a light chain variable (V_{L}) region, wherein:
the V_{H} region is or comprises the sequence set forth in SEQ ID NO: 112, 121, 103 or 130, and
the V_{L} region is or comprises the sequence set forth in SEQ ID NO: 115, 124 or 106.

20. An anti-ROR1 antibody or antigen-binding fragment thereof, comprising: a heavy chain variable (V_{H}) region and a light chain variable (V_{L}) region, wherein:
the V_{H} region and the V_{L} region are or comprise the sequence set forth in SEQ ID NOS: 112 and 115, respectively;
the V_{H} region and the V_{L} region are or comprise the sequence set forth in SEQ ID NOS: 121 and 124, respectively;
the V_{H} region and the V_{L} region are or comprise the sequence set forth in SEQ ID NOS: 103 and 106, respectively; or
the V_{H} region and the V_{L} region are or comprise the sequence set forth in SEQ ID NOS: 130 and 106, respectively.

21. The anti-ROR1 antibody or antigen-binding fragment thereof of any of embodiments 1-20, wherein the V_{H} region and the V_{L} region are or comprise the sequence set forth in SEQ ID NOS: 112 and 115, respectively.

22. The anti-ROR1 antibody or antigen-binding fragment thereof of any of embodiments 1-20, wherein the V_{H} region and the V_{L} region are or comprise the sequence set forth in SEQ ID NOS:121 and 124, respectively.

23. The anti-ROR1 antibody or antigen-binding fragment thereof of any of embodiments 1, 2, 5, 6, 9, 10, 13, 14 and 17-20, wherein the V_{H} region is or comprises the amino acid sequence encoded by SEQ ID NO: 110, 119, 101 or 128 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 110, 119, 101 or 128, and the V_{L} region is or comprises the amino acid sequence encoded by SEQ ID NO: 113, 122 or 104, or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 113, 122 or 104.

24. The anti-ROR1 antibody or antigen-binding fragment thereof of any of embodiments 1, 2, 5, 6, 9, 10, 13, 14, 17-20 and 23, wherein the V_{H} region is or comprises the amino acid sequence encoded by SEQ ID NO: 111, 120, 102 or 129, and the V_{L} region is or comprises the amino acid sequence encoded by SEQ ID NO: 114, 123, 105 or 131.

25. The anti-ROR1 antibody or antigen-binding fragment thereof of any of embodiments 1-24, wherein the V_{H} region is or comprises the amino acid sequence encoded by SEQ ID NO: 110 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 110, and the V_{L} region is or comprises the amino acid sequence encoded by SEQ ID NO: 113, or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 113.

26. The anti-ROR1 antibody or antigen-binding fragment thereof of any of embodiments 1-25, wherein the V_{H} region is or comprises the amino acid sequence encoded by SEQ ID NO: 111, and the V_{L} region is or comprises the amino acid sequence encoded by SEQ ID NO: 114.

27. The anti-ROR1 antibody or antigen-binding fragment thereof of any of embodiments 1-24, wherein the V_{H} region is or comprises the amino acid sequence encoded by SEQ ID NO: 119 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 119, and the V_{L} region is or comprises the amino acid sequence encoded by SEQ ID NO: 122, or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 122.

28. The anti-ROR1 antibody or antigen-binding fragment thereof of any of embodiments 1-25, wherein the V_{H} region is or comprises the amino acid sequence encoded by SEQ ID NO: 120, and the V_{L} region is or comprises the amino acid sequence encoded by SEQ ID NO: 123.

29. The anti-ROR1 antibody or antigen-binding fragment thereof of any of embodiments 1-28, wherein said anti-ROR1 antibody or antigen-binding fragment thereof is isolated.

30. The anti-ROR1 antibody or antigen-binding fragment thereof of any of embodiments 1-29, wherein said anti-ROR1 antibody or antigen-binding fragment thereof is recombinant.

31. The anti-ROR1 antibody or antigen-binding fragment thereof of any of embodiments 1-30, wherein at least a portion of the V_{H} region and the V_{L} region is human or is from a human protein.

32. The anti-ROR1 antibody or antigen-binding fragment thereof of any of embodiments 1-31, wherein the antigen-binding fragment thereof comprises a single chain fragment, optionally a single chain Fv (scFv).

33. The anti-ROR1 antibody or antigen-binding fragment thereof of any of embodiments 1-32, wherein the V_{H} region is amino-terminal to the V_{L} region.

34. The anti-ROR1 antibody or antigen-binding fragment thereof of any of embodiments 1-32, wherein the V_{H} region is carboxy-terminal to the V_{L} region.

35. The anti-ROR1 antibody or antigen-binding fragment thereof of any of embodiments 1-34, when the V_{H} region and the V_{L} region are joined by a flexible linker.

36. The anti-ROR1 antibody or antigen-binding fragment thereof of embodiment 35, wherein the flexible linker comprises the sequence set forth in SEQ ID NO:41.

37. The anti-ROR1 antibody or antigen-binding fragment thereof of any of embodiments 32, 33, 35 and 36, wherein the scFv is or comprises the sequence set forth in SEQ ID NO: 118, 127, 109 or 134, or an amino acid sequence having at least at or about 85%, 86%, 87%, 88%, 89%, 90%, 91%, 92%, 93%, 94%, 95%, 96%, 97%, 98% or 99% sequence identity to SEQ ID NO: 118, 127, 109 or 134.

38. The anti-ROR1 antibody or antigen-binding fragment thereof of any of embodiments 32, 33 and 35-37, wherein the scFv is or comprises the sequence set forth in SEQ ID NO: 118.

39. The anti-ROR1 antibody or antigen-binding fragment thereof of any of embodiments 32, 33 and 35-37, wherein the scFv is or comprises the sequence set forth in SEQ ID NO: 127.

40. The anti-ROR1 antibody or antigen-binding fragment thereof of any of embodiments 32, 33 and 35-37, wherein the scFv is or comprises the amino acid sequence encoded by SEQ ID NO: 116, 125, 107 or 132 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 116, 125, 107 or 132.

41. The anti-ROR1 antibody or antigen-binding fragment thereof of any of embodiments 32, 33, 35-37 and 40, wherein the scFv is or comprises the amino acid sequence encoded by SEQ ID NO: 117, 126, 108 or 133.

42. The anti-ROR1 antibody or antigen-binding fragment thereof of any of embodiments 32-41, wherein the scFv is or comprises the amino acid sequence encoded by SEQ ID NO: 116 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 116.

43. The anti-ROR1 antibody or antigen-binding fragment thereof of any of embodiments 32-42, wherein the scFv is or comprises the amino acid sequence encoded by SEQ ID NO: 117.

44. The anti-ROR1 antibody or antigen-binding fragment thereof of any of embodiments 32-41, wherein the scFv is or comprises the amino acid sequence encoded by SEQ ID NO: 125 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 125.

45. The anti-ROR1 antibody or antigen-binding fragment thereof of any of embodiments 32-41 and 43, wherein the scFv is or comprises the amino acid sequence encoded by SEQ ID NO: 126.

46. The anti-ROR1 antibody or antigen-binding fragment thereof of any of embodiments 1-45, wherein the anti-ROR1 antibody or fragment further comprises at least a portion of an immunoglobulin constant region or a variant thereof.

47. The anti-ROR1 antibody or antigen-binding fragment thereof of embodiment 46, wherein the portion of an immunoglobulin constant region comprises at least a portion of a hinge region or a variant thereof.

48. The anti-ROR1 antibody or antigen-binding fragment thereof of embodiment 46 or embodiment 47, wherein the at least a portion of an immunoglobulin constant region or a variant thereof comprises at least a portion of a C_{H}2 region and/or a C_{H}3 region or a variant thereof.

49. The anti-ROR1 antibody or antigen-binding fragment thereof of any of embodiments 46-48, wherein the at least a portion of an immunoglobulin constant region or a variant thereof is human or from a human protein or a variant thereof.

50. The anti-ROR1 antibody or antigen-binding fragment thereof of any of embodiments 1-49, wherein said anti-ROR1 antibody or antigen-binding fragment thereof specifically binds to a Receptor tyrosine kinase-like orphan receptor 1 (ROR1) protein, optionally a human ROR1.

51. The anti-ROR1 antibody or antigen-binding fragment thereof of embodiment 50, wherein the human ROR1 protein comprises an amino acid sequence set forth in SEQ ID NO: 144, 145 or 146.

52. The anti-ROR1 antibody or antigen-binding fragment thereof of any of embodiments 1-51, wherein said anti-ROR1 antibody or antigen-binding fragment thereof does not bind to, is not cross-reactive to, or binds at a lower level or degree or affinity to a Receptor tyrosine kinase-like orphan receptor 2 (ROR2) protein, optionally a human ROR2.

53. The anti-ROR1 antibody or antigen-binding fragment thereof of any of embodiments 1-52, wherein the extent, level or degree or affinity of binding of said anti-ROR1 antibody or antigen-binding fragment thereof to a human ROR2 is at least at or about 75%, 80%, 90%, 95% or 99% less than the extent, level or degree or affinity of binding to a human ROR1.

54. A single chain cell-surface protein, comprising the anti-ROR1 antibody or antigen-binding fragment thereof of any of embodiments 1-53 and 244-265.

55. A conjugate, comprising the anti-ROR1 antibody or antigen-binding fragment thereof of any of embodiments 1-53 and 244-265 and a heterologous molecule or moiety.

56. The conjugate of embodiment 55, wherein the heterologous molecule or moiety is a therapeutic moiety.

57. An anti-ROR1 chimeric antigen receptor (CAR) comprising an extracellular antigen-binding domain comprising the anti-ROR1 antibody or antigen-binding fragment thereof of any of embodiments 1-53 and 244-265 and an intracellular signaling region.

58. An anti-ROR1 chimeric antigen receptor (CAR) comprising:
an extracellular antigen-binding domain comprising a heavy chain variable (V_{H}) region, and a light chain variable (V_{L}) region, and an intracellular signaling region, wherein the V_{H} region comprises a heavy chain complementarity determining region 1 (CDR-H1) comprising the sequence set forth in SEQ ID NO: 67, 82 or 52, a heavy chain complementarity determining region 2 (CDR-H2) comprising the sequence set forth in SEQ ID NO: 71, 86, 56 or 97, and a heavy chain complementarity determining region 3 (CDR-H3) comprising the sequence set forth in SEQ ID NO: 73, 88, 58 or 99, and the V_{L} region comprises a light chain complementarity determining region 1 (CDR-L1) comprising the sequence set forth in SEQ ID NO: 75, 90 or 60, a light chain complementarity determining region 2 (CDR-L2) comprising the sequence set forth in SEQ ID NO: 77, 92 or 62; and a light chain complementarity determining region 3 (CDR-L3) comprising the sequence set forth in SEQ ID NO: 79, 94 or 64.

59. An anti-ROR1 chimeric antigen receptor (CAR) comprising:
an extracellular antigen-binding domain comprising a heavy chain variable (V_{H}) region, and a light chain variable (V_{L}) region, and an intracellular signaling region, wherein the V_{H} region comprises a heavy chain complementarity determining region 1 (CDR-H1), a heavy chain complementarity determining region 2 (CDR-H2) and a heavy chain complementarity determining region 3 (CDR-H3) comprising the sequence set forth in SEQ ID NOS:67, 71 and 73, respectively, and the V_{L} region comprises a light chain complementarity determining region 1 (CDR-L1), a light chain complementarity determining region 2 (CDR-L2) and a light chain complementarity determining region 3 (CDR-L3) comprising the sequence set forth in SEQ ID NOS:75, 77 and 79, respectively;
the V_{H} region comprises a CDR-H1, a CDR-H2 and a CDR-H3 comprising the sequence set forth in SEQ ID NOS:82, 86 and 88, respectively, and the V_{L} region comprises a CDR-L1, a CDR-L2 and a CDR-L3 comprising the sequence set forth in SEQ ID NOS:90, 92 and 94, respectively;
the V_{H} region comprises a CDR-H1, a CDR-H2 and a CDR-H3 comprising the sequence set forth in SEQ ID NOS:52, 56 and 58, respectively, and the V_{L} region comprises a CDR-L1, a CDR-L2 and a CDR-L3 comprising the sequence set forth in SEQ ID NOS:60, 62 and 64, respectively; or
the V_{H} region comprises a CDR-H1, a CDR-H2 and a CDR-H3 comprising the sequence set forth in SEQ ID NOS:52, 97 and 99, respectively, and the V_{L} region comprises a CDR-L1, a CDR-L2 and a CDR-L3 comprising the sequence set forth in SEQ ID NOS:60, 62 and 64, respectively.

60. The anti-ROR1 chimeric antigen receptor of embodiment 58 or embodiment 59, wherein the V_{H} region comprises a CDR-H1, a CDR-H2 and a CDR-H3 comprising the sequence set forth in SEQ ID NOS:67, 71 and 73, respectively, and the V_{L} region comprises a CDR-L1, a CDR-L2 and a CDR-L3 comprising the sequence set forth in SEQ ID NOS:75, 77 and 79, respectively.

61. The anti-ROR1 chimeric antigen receptor of embodiment 58 or embodiment 59, wherein the V_{H} region comprises a CDR-H1, a CDR-H2 and a CDR-H3 comprising the sequence set forth in SEQ ID NOS:82, 86 and 88, respectively, and the V_{L} region comprises a CDR-L1, a CDR-L2 and a CDR-L3 comprising the sequence set forth in SEQ ID NOS:90, 92 and 94, respectively.

62. An anti-ROR1 chimeric antigen receptor (CAR) comprising:
an extracellular antigen-binding domain comprising a heavy chain variable (V_{H}) region and a light chain variable (V_{L}) region, and an intracellular signaling region, wherein:
the V_{H} region comprises a heavy chain complementarity determining region 1 (CDR-H1), a heavy chain complementarity determining region 2 (CDR-H2) and a heavy chain complementarity determining region 3 (CDR-H3) contained within SEQ ID NO: 112, 121, 103 or 130, and
the V_{L} region comprises a light chain complementarity determining region 1 (CDR-L1), a light chain complementarity determining region 2 (CDR-L2) and a light chain complementarity determining region 3 (CDR-L3) contained within SEQ ID NO: 115, 124 or 106.

63. An anti-ROR1 chimeric antigen receptor (CAR) comprising:
an extracellular antigen-binding domain comprising a heavy chain variable (V_{H}) region and a light chain variable (V_{L}) region, and an intracellular signaling region, wherein:
the V_{H} region comprises a heavy chain complementarity determining region 1 (CDR-H1), a heavy chain complementarity determining region 2 (CDR-H2) and a heavy chain complementarity determining region 3 (CDR-H3) contained within SEQ ID NO:112, and the V_{L} region comprises a light chain complementarity determining region 1 (CDR-L1), a light chain complementarity determining region 2 (CDR-L2) and a light chain complementarity determining region 3 (CDR-L3) contained within SEQ ID NO:115;
the V_{H} region comprises a CDR-H1, a CDR-H2 and a CDR-H3 contained within SEQ ID NO:121, and the V_{L} region comprises a CDR-L1, a CDR-L2 and a CDR-L3 contained within SEQ ID: NO 124;
the V_{H} region comprises a CDR-H1, a CDR-H2 and a CDR-H3 contained within SEQ ID NO:103, and the V_{L} region comprises a CDR-L1, a CDR-L2 and a CDR-L3 contained within SEQ ID: NO 106; or
the V_{H} region comprises a CDR-H1, a CDR-H2 and a CDR-H3 contained within SEQ ID NO:130, and the V_{L} region comprises a CDR-L1, a CDR-L2 and a CDR-L3 contained within SEQ ID: NO 106.

64. The anti-ROR1 chimeric antigen receptor of embodiment 62 or embodiment 63, wherein the V_{H} region comprises a CDR-H1, a CDR-H2 and a CDR-H3 contained within SEQ ID NO:112, and the V_{L} region comprises a CDR-L1, a CDR-L2 and a CDR-L3 contained within SEQ ID: NO 115.

65. The anti-ROR1 chimeric antigen receptor of embodiment 62 or embodiment 63, wherein the V_{H} region comprises a CDR-H1, a CDR-H2 and a CDR-H3 contained within SEQ ID NO:121, and the V_{L} region comprises a CDR-L1, a CDR-L2 and a CDR-L3 contained within SEQ ID: NO 124.

66. The anti-ROR1 chimeric antigen receptor of any of embodiments 58, 59, 62 and 63, wherein:
the V_{H} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 112, 121, 103 or 130, and
the V_{L} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 115, 124 or 106.

67. The anti-ROR1 chimeric antigen receptor of any of embodiments 58, 59, and 62-64, wherein:
the V_{H} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO:112, and the V_{L} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 115;
the V_{H} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO:121, and the V_{L} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO:124;
the V_{H} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO:103, and the V_{L} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO:106; or
the V_{H} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO:130, and the V_{L} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO:106.

68. The anti-ROR1 chimeric antigen receptor of any of embodiments 58-67, wherein the V_{H} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO:112, and the V_{L} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 115.

69. The anti-ROR1 chimeric antigen receptor of any of embodiments 58-67, wherein the V_{H} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO:121, and the V_{L} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO:124.

70. An anti-ROR1 chimeric antigen receptor (CAR) comprising:
an extracellular antigen-binding domain comprising a heavy chain variable (V_{H}) region and a light chain variable (V_{L}) region, and an intracellular signaling region, wherein:
the V_{H} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 112, 121, 103 or 130, and
the V_{L} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 115, 124 or 106.

71. An anti-ROR1 chimeric antigen receptor (CAR) comprising:
an extracellular antigen-binding domain comprising a heavy chain variable (V_{H}) region and a light chain variable (V_{L}) region, and an intracellular signaling region, wherein:
the V_{H} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO:112, and the V_{L} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 115;
the V_{H} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO:121, and the V_{L} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO:124;
the V_{H} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO:103, and the V_{L} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO:106; or
the V_{H} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO:130, and the V_{L} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO:106.

72. The anti-ROR1 chimeric antigen receptor of embodiment 70 or embodiment 71, wherein the V_{H} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO:112, and the V_{L} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 115.

73. The anti-ROR1 chimeric antigen receptor of embodiment 70 or embodiment 71, wherein the V_{H} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO:121, and the V_{L} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO:124.

74. The anti-ROR1 chimeric antigen receptor of any of embodiments 58, 59, 62, 63, 66, 67, 70 and 71, wherein:
the V_{H} region is or comprises the sequence set forth in SEQ ID NO: 112, 121, 103 or 130, and
the V_{L} region is or comprises the sequence set forth in SEQ ID NO: 115, 124 or 106.

75. The anti-ROR1 chimeric antigen receptor of any of embodiments 58, 59, 62, 63, 66, 67, 70, 71 and 74, wherein:
the V_{H} region and the V_{L} region are or comprise the sequence set forth in SEQ ID NOS: 112 and 115, respectively;
the V_{H} region and the V_{L} region are or comprise the sequence set forth in SEQ ID NOS: 121 and 124, respectively;
the V_{H} region and the V_{L} region are or comprise the sequence set forth in SEQ ID NOS: 103 and 106, respectively; or
the V_{H} region and the V_{L} region are or comprise the sequence set forth in SEQ ID NOS: 130 and 106, respectively.

76. An anti-ROR1 chimeric antigen receptor (CAR) comprising:
an extracellular antigen-binding domain comprising a heavy chain variable (V_{H}) region and a light chain variable (V_{L}) region, and an intracellular signaling region, wherein:
the V_{H} region is or comprises the sequence set forth in SEQ ID NO: 112, 121, 103 or 130, and
the V_{L} region is or comprises the sequence set forth in SEQ ID NO: 115, 124 or 106.

77. An anti-ROR1 chimeric antigen receptor (CAR) comprising:
an extracellular antigen-binding domain comprising a heavy chain variable (V_{H}) region and a light chain variable (V_{L}) region, and an intracellular signaling region, wherein:
the V_{H} region and the V_{L} region are or comprise the sequence set forth in SEQ ID NOS: 112 and 115, respectively;
the V_{H} region and the V_{L} region are or comprise the sequence set forth in SEQ ID NOS: 121 and 124, respectively;
the V_{H} region and the V_{L} region are or comprise the sequence set forth in SEQ ID NOS: 103 and 106, respectively; or
the V_{H} region and the V_{L} region are or comprise the sequence set forth in SEQ ID NOS: 130 and 106, respectively.

78. The anti-ROR1 chimeric antigen receptor of any of embodiments 58-77, wherein the V_{H} region and the V_{L} region are or comprise the sequence set forth in SEQ ID NOS: 112 and 115, respectively.

79. The anti-ROR1 chimeric antigen receptor of any of embodiments 58-77, wherein the V_{H} region and the V_{L} region are or comprise the sequence set forth in SEQ ID NOS:121 and 124, respectively.

80. The anti-ROR1 chimeric antigen receptor of any of embodiments 58, 59, 62, 63, 66, 67, 70, 71 and 74-77, wherein the V_{H} region is or comprises the amino acid sequence encoded by SEQ ID NO: 110, 119, 101 or 128 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 110, 119, 101 or 128, and the V_{L} region is or comprises the amino acid sequence encoded by SEQ ID NO: 113, 122 or 104, or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 113, 122 or 104.

81. The anti-ROR1 chimeric antigen receptor of any of embodiments 58, 59, 62, 63, 66, 67, 70, 71, 74-77 and 80, wherein the V_{H} region is or comprises the amino acid sequence encoded by SEQ ID NO: 111, 120, 102 or 129, and the V_{L} region is or comprises the amino acid sequence encoded by SEQ ID NO: 114, 123, 105 or 131.

82. The anti-ROR1 chimeric antigen receptor of any of embodiments 58-81, wherein the V_{H} region is or comprises the amino acid sequence encoded by SEQ ID NO: 110 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 110, and the V_{L} region is or comprises the amino acid sequence encoded by SEQ ID NO: 113, or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 113.

83. The anti-ROR1 chimeric antigen receptor of any of embodiments 58-82, wherein the V_{H} region is or comprises the amino acid sequence encoded by SEQ ID NO: 111, and the V_{L} region is or comprises the amino acid sequence encoded by SEQ ID NO: 114.

84. The anti-ROR1 chimeric antigen receptor of any of embodiments 58-81, wherein the V_{H} region is or comprises the amino acid sequence encoded by SEQ ID NO: 119 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 119, and the V_{L} region is or comprises the amino acid sequence encoded by SEQ ID NO: 122, or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 122.

85. The anti-ROR1 chimeric antigen receptor of any of embodiments 58-82, wherein the V_{H} region is or comprises the amino acid sequence encoded by SEQ ID NO: 120, and the V_{L} region is or comprises the amino acid sequence encoded by SEQ ID NO: 123.

86. The anti-ROR1 chimeric antigen receptor of any of embodiments 58-85, wherein said anti-ROR1 antibody or antigen-binding fragment thereof is isolated.

87. The anti-ROR1 chimeric antigen receptor of any of embodiments 58-86, wherein said anti-ROR1 antibody or antigen-binding fragment thereof is recombinant.

88. The anti-ROR1 chimeric antigen receptor of any of embodiments 58-87, wherein at least a portion of the V_{H} region and the V_{L} region is human or is from a human protein.

89. The anti-ROR1 chimeric antigen receptor of any of embodiments 58-88, wherein the antigen-binding fragment thereof comprises a single chain fragment, optionally a single chain Fv (scFv).

90. The anti-ROR1 chimeric antigen receptor of any of embodiments 58-89, wherein the V_{H} region is amino-terminal to the V_{L} region.

91. The anti-ROR1 chimeric antigen receptor of any of embodiments 58-89, wherein the V_{H} region is carboxy-terminal to the V_{L} region.

92. The anti-ROR1 chimeric antigen receptor of any of embodiments 58-91, when the V_{H} region and the V_{L} region are joined by a flexible linker.

93. The anti-ROR1 chimeric antigen receptor of embodiment 92, wherein the flexible linker comprises the sequence set forth in SEQ ID NO:41.

94. The anti-ROR1 chimeric antigen receptor of any of embodiments 89, 90, 92 and 93, wherein the scFv is or comprises the sequence set forth in SEQ ID NO: 118, 127, 109 or 134, or an amino acid sequence having at least at or about 85%, 86%, 87%, 88%, 89%, 90%, 91%, 92%, 93%, 94%, 95%, 96%, 97%, 98% or 99% sequence identity to SEQ ID NO: 118, 127, 109 or 134.

95. The anti-ROR1 chimeric antigen receptor of any of embodiments 89, 90 and 92-94, wherein the scFv is or comprises the sequence set forth in SEQ ID NO: 118.

96. The anti-ROR1 chimeric antigen receptor of any of embodiments 89, 90 and 92-94, wherein the scFv is or comprises the sequence set forth in SEQ ID NO: 127.

97. The anti-ROR1 chimeric antigen receptor of any of embodiments 89, 90 and 92-94, wherein the scFv is or comprises the amino acid sequence encoded by SEQ ID NO: 116, 125, 107 or 132 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 116, 125, 107 or 132.

98. The anti-ROR1 chimeric antigen receptor of any of embodiments 89, 90, 92-94 and 40, wherein the scFv is or comprises the amino acid sequence encoded by SEQ ID NO: 117, 126, 108 or 133.

99. The anti-ROR1 chimeric antigen receptor of any of embodiments 89-98, wherein the scFv is or comprises the amino acid sequence encoded by SEQ ID NO: 116 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 116.

100. The anti-ROR1 chimeric antigen receptor of any of embodiments 89-99, wherein the scFv is or comprises the amino acid sequence encoded by SEQ ID NO: 117.

101. The anti-ROR1 chimeric antigen receptor of any of embodiments 89-98, wherein the scFv is or comprises the amino acid sequence encoded by SEQ ID NO: 125 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 125.

102. The anti-ROR1 chimeric antigen receptor of any of embodiments 89-98 and 100, wherein the scFv is or comprises the amino acid sequence encoded by SEQ ID NO: 126.

103. The anti-ROR1 chimeric antigen receptor of any of embodiments 58-102, further comprising a spacer.

104. The anti-ROR1 chimeric antigen receptor of embodiment 103, wherein the spacer comprises at least a portion of an immunoglobulin or a variant thereof.

105. The anti-ROR1 chimeric antigen receptor of embodiment 103 or embodiment 104, wherein the spacer comprises at least a portion of a hinge region of an immunoglobulin or a variant thereof.

106. The anti-ROR1 chimeric antigen receptor of any of embodiments 103-105, wherein the spacer is less than at or about 15 amino acids in length.

107. The anti-ROR1 chimeric antigen receptor of any of embodiments 103-106, wherein the spacer is or comprises an amino acid sequence having at least at or about 85%, 86%, 87%, 88%, 89%, 90%, 91%, 92%, 93%, 94%, 95%, 96%, 97%, 98% or 99% identity to SEQ ID NO:1, 26, 27, 29, 31, 32, 33 or 135.

108. The anti-ROR1 chimeric antigen receptor of any of embodiments 105-107, wherein the at least a portion of a hinge region comprises all or a portion of an IgG4 hinge region, optionally a human IgG4 hinge region, or a variant thereof.

109. The anti-ROR1 chimeric antigen receptor of any of embodiments 105-107, wherein the at least a portion of a hinge region comprises all or a portion of an IgG2 hinge region, optionally a human IgG2 hinge region, or a variant thereof.

110. The anti-ROR1 chimeric antigen receptor of any of embodiments 103-109, wherein the spacer is or comprises the sequence set forth in SEQ ID NO: 135.

111. The anti-ROR1 chimeric antigen receptor of any of embodiments 103-110, wherein the spacer is or comprises the amino acid sequence encoded by SEQ ID NO: 192 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 192.

112. The anti-ROR1 chimeric antigen receptor of any of embodiments 103-111, wherein the spacer is or comprises the amino acid sequence encoded by SEQ ID NO: 136.

113. The anti-ROR1 chimeric antigen receptor of any of embodiments 103-112, wherein the spacer comprises at least a portion of a CH3 region of an immunoglobulin or a variant thereof.

114. The anti-ROR1 chimeric antigen receptor of any of embodiments 103-113, wherein the at least a portion of a CH3 region comprises all or a portion of an IgG4 CH3 and/or an IgG2 CH3, wherein the IgG4 CH3 is optionally a human IgG4 CH3 and the IgG2 CH3 is optionally a human IgG2 CH3.

115. The anti-ROR1 chimeric antigen receptor of any of embodiments 103-114, wherein the spacer comprises at least a portion of a hinge region and at least a portion of a CH3 region of an immunoglobulin or a variant thereof.

116. The anti-ROR1 chimeric antigen receptor of any of embodiments 103-115, wherein the spacer is at or about 111, 112, 113, 114, 115, 116, 117, 118, 119, 120, 121, 122, 123, 124 or 125 amino acids in length, or has a length between any of the foregoing.

117. The anti-ROR1 chimeric antigen receptor of any of embodiments 103-116, wherein the spacer is at or about 120 amino acids in length.

118. The anti-ROR1 chimeric antigen receptor of any of embodiments 103-117, wherein the spacer is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 138.

119. The anti-ROR1 chimeric antigen receptor of any of embodiments 103-118, wherein the spacer is or comprises the sequence set forth in SEQ ID NO:138.

120. The anti-ROR1 chimeric antigen receptor of any of embodiments 103-119, wherein the spacer is or comprises the amino acid sequence encoded by SEQ ID NO: 193 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 193.

121. The anti-ROR1 chimeric antigen receptor of any of embodiments 103-120, wherein the spacer is or comprises the amino acid sequence encoded by SEQ ID NO: 139.

122. The anti-ROR1 chimeric antigen receptor of any of embodiments 103-121, wherein the spacer comprises at least a portion of a CH2 of an immunoglobulin or a variant thereof.

123. The anti-ROR1 chimeric antigen receptor of any of embodiments 103-122, wherein the at least a portion of a CH2 region comprises all or a portion of an IgG4 CH2 and/or an IgG2 CH2, wherein the IgG4 CH2 is optionally a human IgG4 CH2 and the IgG2 CH2 is optionally a human IgG2 CH2.

124. The anti-ROR1 chimeric antigen receptor of any of embodiments 103-123, wherein the spacer comprises at least a portion of a hinge region, at least a portion of a CH2 and at least a portion of a CH3 region of an immunoglobulin or a variant thereof.

125. The anti-ROR1 chimeric antigen receptor of any of embodiments 103-124, wherein the spacer is at or about 130, 140, 150, 160, 170, 180, 190, 200, 210, 220, 221, 222, 223, 224, 225, 226, 227, 228, 229 or 230 amino acids in length, or has a length between any of the foregoing.

126. The anti-ROR1 chimeric antigen receptor of any of embodiments 103-125, wherein one or more of the hinge region, the C_{H}2 region and the C_{H}3 region comprises all or a portion of a C_{H}2 region and all or a portion of a C_{H}3 region from human IgG4.

127. The anti-ROR1 chimeric antigen receptor of any of embodiments 103-125, wherein one or more of the hinge region, the C_{H}2 region and the C_{H}3 region is chimeric and comprises a hinge, a C_{H}2 region and a C_{H}3 region from human IgG4 and human IgG2.

128. The anti-ROR1 chimeric antigen receptor of any of embodiments 103-125, wherein the spacer comprises a IgG4/2 chimeric hinge region or a modified IgG4 hinge region comprising at least one amino acid replacement compared to a human IgG4 hinge; an IgG2/4 chimeric C_{H}2 region; and an IgG4 C_{H}3 region.

129. The anti-ROR1 chimeric antigen receptor of any of embodiments 103-125, wherein the spacer is or comprises an amino acid sequence having at least at or about 85%, 86%, 87%, 88%, 89%, 90%, 91%, 92%, 93%, 94%, 95%, 96%, 97%, 98% or 99% identity to SEQ ID NO:194.

130. The anti-ROR1 chimeric antigen receptor of any of embodiments 103-129, wherein the spacer is or comprises the sequence set forth in SEQ ID NO: 194.

131. The anti-ROR1 chimeric antigen receptor of any of embodiments 103-130, wherein the spacer is or comprises the amino acid sequence encoded by SEQ ID NO: 195 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 195.

132. The anti-ROR1 chimeric antigen receptor of any of embodiments 103-131, wherein the spacer is or comprises the amino acid sequence encoded by SEQ ID NO: 196.

133. The anti-ROR1 chimeric antigen receptor of any of embodiments 58-132, wherein the intracellular signaling region comprises an intracellular signaling domain.

134. The anti-ROR1 chimeric antigen receptor of embodiment 133, wherein the intracellular signaling domain is capable of inducing a primary activation signal in a T cell, is a T cell receptor (TCR) component and/or comprises an immunoreceptor tyrosine-based activation motif (ITAM).

135. The anti-ROR1 chimeric antigen receptor of embodiment 133 or embodiment 134, wherein the intracellular signaling domain is or comprises a cytoplasmic signaling domain of a CD3-zeta (CD3ζ chain or a functional variant or signaling portion thereof.

136. The anti-ROR1 chimeric antigen receptor of any of embodiments 133-135, wherein the intracellular signaling domain is human or is from a human protein.

137. The anti-ROR1 chimeric antigen receptor of any of embodiments 133-136, wherein the intracellular signaling domain is or comprises the sequence set forth in SEQ ID NO:13, 14 or 15, or an amino acid sequence having at least at or about 90%, 91%, 92%, 93%, 94%, 95%, 96%, 97%, 98% or 99% sequence identity to SEQ ID NO:13, 14 or 15.

138. The anti-ROR1 chimeric antigen receptor of any of embodiments 133-137, wherein the intracellular signaling domain is or comprises the sequence set forth in SEQ ID NO:13.

139. The anti-ROR1 chimeric antigen receptor of any of embodiments 133-138, wherein the intracellular signaling region further comprises a costimulatory signaling region.

140. The anti-ROR1 chimeric antigen receptor of embodiment 139, wherein the costimulatory signaling region comprises an intracellular signaling domain of a T cell costimulatory molecule or a signaling portion thereof.

141. The anti-ROR1 chimeric antigen receptor of embodiment 139 or embodiment 140, wherein the costimulatory signaling region comprises an intracellular signaling domain of CD28, 4-1BB, or ICOS, or a signaling portion thereof.

142. The anti-ROR1 chimeric antigen receptor of any of embodiments 140-141, wherein the costimulatory signaling region is human or is from a human protein.

143. The anti-ROR1 chimeric antigen receptor of any of embodiments 140-142, wherein the costimulatory signaling region comprises an intracellular signaling domain of CD28.

144. The anti-ROR1 chimeric antigen receptor of any of embodiments 140-143, wherein the costimulatory signaling region is or comprises the sequence set forth in SEQ ID NO:10 or an amino acid sequence having at least at or about 90%, 91%, 92%, 93%, 94%, 95%, 96%, 97%, 98% or 99% sequence identity to SEQ ID NO:10.

145. The anti-ROR1 chimeric antigen receptor of any of embodiments 140-144, wherein the costimulatory signaling region comprises an intracellular signaling domain of 4-1BB.

146. The anti-ROR1 chimeric antigen receptor of any of embodiments 140-142 and 145, wherein the costimulatory signaling region is or comprises the sequence set forth in SEQ ID NO:12 or an amino acid sequence having at least at or about 90%, 91%, 92%, 93%, 94%, 95%, 96%, 97%, 98% or 99% sequence identity to SEQ ID NO: 12.

147. The anti-ROR1 chimeric antigen receptor of any of embodiments 58-146, further comprising a transmembrane region.

148. The anti-ROR1 chimeric antigen receptor of any of embodiments 140-147, wherein the costimulatory signaling region is between the transmembrane region and the intracellular signaling domain.

149. The anti-ROR1 chimeric antigen receptor of embodiment 147 or embodiment 148, wherein the transmembrane region is or comprises a transmembrane domain from CD4, CD28, or CD8.

150. The anti-ROR1 chimeric antigen receptor of any of embodiments 147-149, wherein the transmembrane region is or comprises a transmembrane domain derived from CD28.

151. The anti-ROR1 chimeric antigen receptor of any of embodiments 147-150, wherein the transmembrane region is human or is from a human protein.

152. The anti-ROR1 chimeric antigen receptor of any of embodiments 147-151, wherein the transmembrane domain is or comprises SEQ ID NO: 8 or an amino acid sequence having at least at or about 90%, 91%, 92%, 93%, 94%, 95%, 96%, 97%, 98% or 99% sequence identity to SEQ ID NO: 8.

153. The anti-ROR1 chimeric antigen receptor of any of embodiments 147-152, wherein the encoded chimeric antigen receptor comprises from its N to C terminus in order: the extracellular antigen-binding domain, the spacer, the transmembrane region and the intracellular signaling region.

154. The anti-ROR1 chimeric antigen receptor of any of embodiments 58-153, wherein the encoded chimeric antigen receptor comprises, from its N to C terminus in order: an extracellular antigen-binding domain comprising an scFv, a spacer comprising a modified IgG4 hinge, optionally comprising the sequence set forth in SEQ ID NO:135; a transmembrane domain, optionally a transmembrane domain from a human CD28; and an intracellular signaling region comprising a cytoplasmic signaling domain of a CD3-zeta (CD3ζ chain and an intracellular signaling domain of a costimulatory signaling region, optionally comprising an intracellular signaling domain of 4-1BB.

155. The anti-ROR1 chimeric antigen receptor of any of embodiments 58-153, wherein the encoded chimeric antigen receptor comprises, from its N to C terminus in order: an extracellular antigen-binding domain comprising an scFv, a spacer comprising a modified IgG4 hinge-C_{H}3, optionally comprising the sequence set forth in SEQ ID NO: 138; a transmembrane domain, optionally a transmembrane domain from a human CD28; and an intracellular signaling region comprising a cytoplasmic signaling domain of a CD3-zeta (CD3ζ chain and an intracellular signaling domain of a costimulatory signaling region, optionally comprising an intracellular signaling domain of 4-1BB.

156. The anti-ROR1 chimeric antigen receptor of any of embodiments 58-155, wherein the anti-ROR1 chimeric antigen receptor is or comprises the sequence set forth in SEQ ID NO: 184, 185, 186, 187, 188 or 189 or a sequence that exhibits at least 85%, 86%, 87%, 88%, 89%, 90%, 91%, 92%, 93%, 94%, 95%, 96%, 97%, 98%, or 99% sequence identity to the sequence set forth in SEQ ID NO: 184, 185, 186, 187, 188 or 189.

157. The anti-ROR1 chimeric antigen receptor of any of embodiments 58-156, wherein the anti-ROR1 chimeric antigen receptor is or comprises the sequence set forth in SEQ ID NO: 184 or a sequence that exhibits at least 85%, 86%, 87%, 88%, 89%, 90%, 91%, 92%, 93%, 94%, 95%, 96%, 97%, 98%, or 99% sequence identity to the sequence set forth in SEQ ID NO: 184.

158. The anti-ROR1 chimeric antigen receptor of any of embodiments 58-157, wherein the anti-ROR1 chimeric antigen receptor is or comprises the sequence set forth in SEQ ID NO: 184.

159. The anti-ROR1 chimeric antigen receptor of any of embodiments 58-156, wherein the anti-ROR1 chimeric antigen receptor is or comprises the sequence set forth in SEQ ID NO: 185 or a sequence that exhibits at least 85%, 86%, 87%, 88%, 89%, 90%, 91%, 92%, 93%, 94%, 95%, 96%, 97%, 98%, or 99% sequence identity to the sequence set forth in SEQ ID NO:185.

160. The anti-ROR1 chimeric antigen receptor of any of embodiments 58-156 and 159, wherein the anti-ROR1 chimeric antigen receptor is or comprises the sequence set forth in SEQ ID NO: 185.

161. The anti-ROR1 chimeric antigen receptor of any of embodiments 58-156, wherein the anti-ROR1 chimeric antigen receptor is encoded by the sequence set forth in SEQ ID NO: 156, 157, 158, 159, 160 or 161 or a sequence that exhibits at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 156, 157, 158, 159, 160 or 161.

162. The anti-ROR1 chimeric antigen receptor of any of embodiments 58-158 and 161, wherein the anti-ROR1 chimeric antigen receptor is encoded by the sequence set forth in SEQ ID NO: 156 or a sequence that exhibits at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 156.

163. The anti-ROR1 chimeric antigen receptor of any of embodiments 58-158, 161 and 162, wherein the anti-ROR1 chimeric antigen receptor is encoded by the sequence set forth in SEQ ID NO: 156.

164. The anti-ROR1 chimeric antigen receptor of any of embodiments 58-156 and 159-161, wherein the anti-ROR1 chimeric antigen receptor is encoded by the sequence set forth in SEQ ID NO: 157 or a sequence that exhibits at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 157.

165. The anti-ROR1 chimeric antigen receptor of any of embodiments 58-156, 159-161 and 164, wherein the anti-ROR1 chimeric antigen receptor is encoded by the sequence set forth in SEQ ID NO: 157.

166 The anti-ROR1 chimeric antigen receptor of any of embodiments 58-165, wherein said anti-ROR1 chimeric antigen receptor specifically binds to a receptor tyrosine kinase-like orphan receptor 1 (ROR1) protein, optionally a human ROR1.

167. The anti-ROR1 chimeric antigen receptor of embodiment 166, wherein the human ROR1 protein comprises an amino acid sequence set forth in SEQ ID NO: 144, 145 or 146.

168. The anti-ROR1 chimeric antigen receptor of any of embodiments 58-167, wherein said anti-ROR1 chimeric antigen receptor does not bind to, is not cross-reactive to, or binds at a lower level or degree or affinity to a receptor tyrosine kinase-like orphan receptor 2 (ROR2) protein, optionally a human ROR2.

169. The anti-ROR1 chimeric antigen receptor of any of embodiments 58-168, wherein the extent, level or degree or affinity of binding of said anti-ROR1 chimeric antigen receptor to a human ROR2 is at least at or about 75%, 80%, 90%, 95% or 99% less than the extent, level or degree or affinity of binding to a human ROR1, optionally under the same or substantially the same conditions or assay.

170. The anti-ROR1 chimeric antigen receptor of any of embodiments 58-169, wherein said anti-ROR1 chimeric antigen receptor exhibits the same, substantially the same or lower level or degree of signaling or activity in the presence of a ROR2 protein, optionally a human ROR2, compared to the level or degree of signaling or activity in the presence of a ROR1 protein, optionally a human ROR1, optionally under the same or substantially the same conditions or assay.

171. The anti-ROR1 chimeric antigen receptor of any of embodiments 58-170, wherein said anti-ROR1 chimeric antigen receptor exhibits a level or degree of signaling or activity in the presence of a human ROR2 that is at least at or about 75%, 80%, 90%, 95% or 99% less than the level or degree of signaling or activity in the presence of a human ROR1, optionally under the same or substantially the same conditions or assay.

172. The anti-ROR1 chimeric antigen receptor of any of embodiments 58-171, wherein said anti-ROR1 chimeric antigen receptor exhibits the same, substantially the same or lower level or degree of signaling or activity in the presence of a ROR2 protein, optionally a human ROR2, compared to a reference ROR1-specific chimeric antigen receptor, optionally under the same or substantially the same conditions or assay.

173. The anti-ROR1 chimeric antigen receptor of any of embodiments 58-172, wherein said anti-ROR1 chimeric antigen receptor exhibits the same, substantially the same or lower level or degree of signaling or activity in the presence of a ROR2 protein, optionally a human ROR2, compared to a reference ROR1-specific chimeric antigen receptor, optionally under the same or substantially the same conditions or assay.

174. The anti-ROR1 chimeric antigen receptor of any of embodiments 58-173, wherein said anti-ROR1 chimeric antigen receptor exhibits the same, substantially the same or higher antigen-specific signaling and/or antigen dependent activity or signaling compared to a reference ROR1-specific chimeric antigen receptor, optionally under the same or substantially the same conditions or assay.

175. The anti-ROR1 chimeric antigen receptor of any of embodiments 58-174, wherein said anti-ROR1 chimeric antigen receptor exhibits the same, substantially the same or lower tonic signaling and/or antigen independent activity or signaling compared to a reference ROR1-specific chimeric antigen receptor, optionally under the same or substantially the same conditions or assay.

176. The anti-ROR1 chimeric antigen receptor of any of embodiments 58-175, wherein said anti-ROR1 chimeric antigen receptor exhibits a level or degree of tonic signaling and/or antigen independent activity or signaling that is at least at or about 75%, 80%, 90%, 95% or 99% less than the level or degree of tonic signaling and/or antigen independent activity of a reference ROR1-specific chimeric antigen receptor, optionally under the same or substantially the same conditions or assay.

177. The anti-ROR1 chimeric antigen receptor of any of embodiments 172-176, wherein the reference ROR1-specific chimeric antigen receptor comprises the anti-ROR1 antibody R12 or the anti-ROR1 antibody 2A2 or an antigen-binding fragment thereof, optionally an scFv from R12 or 2A2.

178. A polynucleotide comprising a nucleic acid encoding the anti-ROR1 antibody or antigen-binding domain thereof of any of embodiments 1-53 and 244-265, the single chain cell surface protein of embodiment 54, the conjugate of embodiment 55 or embodiment 56 or the anti-ROR1 chimeric antigen receptor of any of embodiments 57-177 and 266-297.

179. The polynucleotide of embodiment 178, wherein said polynucleotide comprises a nucleic acid encoding the V_{H} comprising the sequence set forth in SEQ ID NO: 110, 119, 101 or 128 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 110, 119, 101 or 128, and a nucleic acid encoding the V_{L} comprising the sequence set forth in SEQ ID NO: 113, 122 or 104, or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 113, 122 or 104.

180. The polynucleotide of embodiment 178 or embodiment 179, wherein said polynucleotide comprises a nucleic acid encoding the V_{H} comprising the sequence set forth in SEQ ID NO: 110 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 110, and a nucleic acid encoding the V_{L} comprising the sequence set forth in SEQ ID NO: 113, or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 113.

181. The polynucleotide of embodiment 178 or embodiment 179, wherein said polynucleotide comprises a nucleic acid encoding the V_{H} comprising the sequence set forth in SEQ ID NO: 119 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 119, and a nucleic acid encoding the V_{L} comprising the sequence set forth in SEQ ID NO: 122, or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 122.

182. The polynucleotide of any of embodiments 178-180, wherein said polynucleotide comprises a nucleic acid encoding the scFv comprising the sequence set forth in SEQ ID NO: 116, 125, 107 or 132 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 116, 125, 107 or 132.

183. The polynucleotide of any of embodiments 178-180 and 182, wherein said polynucleotide comprises a nucleic acid encoding the scFv comprising the sequence set forth in SEQ ID NO: 116 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 116.

184. The polynucleotide of any of embodiments 178-180 and 182, wherein said polynucleotide comprises a nucleic acid encoding the scFv comprising the sequence set forth in SEQ ID NO: 125 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 125.

185. The polynucleotide of any of embodiments 178-184, wherein said polynucleotide comprises a nucleic acid encoding the spacer comprising the sequence set forth in SEQ ID NO: 192 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 192.

186. The polynucleotide of any of embodiments 178-184, wherein said polynucleotide comprises a nucleic acid encoding the spacer comprising the sequence set forth in SEQ ID NO: 193 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 193.

187. The polynucleotide of any of embodiments 178-184, wherein said polynucleotide comprises a nucleic acid encoding the spacer comprising the sequence set forth in SEQ ID NO: 195 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 195.

188. The polynucleotide of embodiment 187, wherein the nucleic acid encoding the anti-ROR1 antibody or antigen-binding domain thereof, the single chain cell surface protein, the conjugate or the anti-ROR1 chimeric antigen receptor comprises at least one modified splice donor and/or splice acceptor site, said modified splice donor and/or acceptor site comprising one or more nucleotide modifications corresponding to a reference splice donor site and/or reference splice acceptor site.

189. The polynucleotide of embodiment 188, wherein the one or more nucleotide modifications comprise a nucleic acid substitution.

190. The polynucleotide of embodiment 188 or embodiment 189, wherein the reference splice donor and/or reference splice acceptor sites are canonical, non-canonical, or cryptic splice sites.

191. The polynucleotide of any of embodiment 188-190, wherein:
the reference splice donor and/or reference splice acceptor site(s) has a splice site prediction score of at least at or about 0.4, 0.5, 0.6, 0.70, 0.75, 0.80, 0.85, 0.90, 0.95, 0.99 or 1.0; and/or
the reference splice donor and/or reference splice acceptor site(s) is/are predicted to be involved in a splice event with a probability of at least at or about 40%, 50%, 60%, 70%, 75%, 80%, 85%, 90%, 95%, 99% or 100%.

192. The polynucleotide of any of embodiment 188-191, wherein:
the reference splice donor and/or reference splice acceptor site(s) has a splice site prediction score of at least at or about 0.70, 0.75, 0.80, 0.85, 0.90, 0.95, 0.99 or 1.0; and/or
the reference splice donor and/or reference splice acceptor site(s) is/are predicted to be involved in a splice event with a probability of at least at or about 70%, 75%, 80%, 85%, 90%, 95%, 99% or 100%.

193. The polynucleotide of any of embodiments 188-192, wherein at least one of the one or more nucleotide modifications are within 1, 2, 3, 4, 5, 6, 7, 8, 9 or 10 residues of the splice site junction of the reference splice acceptor and/or reference splice donor site.

194. The polynucleotide of any of embodiments 188-193, wherein the one or more nucleotide modifications is silent and/or results in a degenerate codon and/or does not change the amino acid sequence of the encoded protein.

195. The polynucleotide of any of embodiments 188-194, wherein upon expression of the polynucleotide in a cell, the transcribed RNA, optionally messenger RNA (mRNA), from the polynucleotide, exhibits at least at or about 70%, at or about 75%, at or about 80%, at or about 85%, at or about 90%, or at or about 95% RNA homogeneity.

196. The polynucleotide of any of embodiments 188-195, wherein, upon expression in a cell, the transcribed RNA, optionally messenger RNA (mRNA), from the polynucleotide exhibits reduced heterogeneity compared to the heterogeneity of the mRNA transcribed from a reference polynucleotide, said reference polynucleotide encoding the same amino acid sequence as the polynucleotide, wherein the reference polynucleotide differs by the presence of one or more splice donor site and/or one or more splice acceptor site in the nucleic acid encoding the spacer and/or comprises one or more nucleotide modifications compared to the polynucleotide.

197. The polynucleotide of embodiment 196, wherein the RNA heterogeneity is reduced by greater than at or about 10%, 15%, 20%, 25%, 30%, 40% or 50% or more.

198. The polynucleotide of embodiment 196 or embodiment 197, wherein the transcribed RNA, optionally messenger RNA (mRNA), from the reference polynucleotide exhibits greater than at or about 10%, 15%, 20%, 25%, 30%, 40% or 50% or more RNA heterogeneity.

199. The polynucleotide of any of embodiments 188-198, wherein the RNA homogeneity and/or heterogeneity is determined by agarose gel electrophoresis, chip-based capillary electrophoresis, analytical ultracentrifugation, field flow fractionation, or liquid chromatography.

200. The polynucleotide of any of embodiments 188-199, wherein the polynucleotide is codon-optimized for expression in a human cell.

201. The polynucleotide of any of embodiments 178, 179, 182, 185, 186 and 188-200, wherein said polynucleotide comprises a nucleic acid encoding the V_{H} comprising the sequence set forth in SEQ ID NO: 111, 120, 102 or 129, and a nucleic acid encoding the V_{L} comprising the sequence set forth in SEQ ID NO: 114, 123, 105 or 131.

202. The polynucleotide of any of embodiments 178-180, 182, 183, 185, 186 and 198-201, wherein said polynucleotide comprises a nucleic acid encoding the V_{H} comprising the sequence set forth in SEQ ID NO: 111, and a nucleic acid encoding the V_{L} comprising the sequence set forth in SEQ ID NO: 114.

203. The polynucleotide of any of embodiments 178, 179, 181, 182, 184-186 and 198-201, wherein said polynucleotide comprises a nucleic acid encoding the V_{H} comprising the sequence set forth in SEQ ID NO: 120, and a nucleic acid encoding the V_{L} comprising the sequence set forth in SEQ ID NO: 123.

204. The polynucleotide of any of embodiments 178, 179, 182, 185, 186 and 188-201, wherein said polynucleotide comprises a nucleic acid encoding the scFv comprising the sequence set forth in SEQ ID NO: 117, 126, 108 or 133.

205. The polynucleotide of any of embodiments 178-180, 182, 183, 185, 186, 198-201 and 204, wherein said polynucleotide comprises a nucleic acid encoding the scFv comprising the sequence set forth in SEQ ID NO: 117.

206. The polynucleotide of any of embodiments 178, 179, 181, 182, 184-186, 198-201 and 204, wherein said polynucleotide comprises a nucleic acid encoding the scFv comprising the sequence set forth in SEQ ID NO: 126.

207. The polynucleotide of any of embodiments 178-185 and 188-206, wherein said polynucleotide comprises a nucleic acid encoding the spacer comprising the sequence set forth in SEQ ID NO:136.

208. The polynucleotide of any of embodiments 178-184, 186 and 188-206 wherein said polynucleotide comprises a nucleic acid encoding the spacer comprising the sequence set forth in SEQ ID NO:139.

209. The polynucleotide of any of embodiments 178-184 and 187-206, wherein said polynucleotide comprises a nucleic acid encoding the spacer comprising the sequence set forth in SEQ ID NO:196.

210. The polynucleotide of any of embodiments 178-186 and 188-209, wherein said polynucleotide comprises the sequence set forth in SEQ ID NO: 156, 157, 158, 159, 160 or 161 or a sequence that exhibits at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 156, 157, 158, 159, 160 or 161.

211. The polynucleotide of any of embodiments 178-186 and 188-210, wherein said polynucleotide comprises the sequence set forth in SEQ ID NO: 156 or a sequence that exhibits at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 156.

212. The polynucleotide of any of embodiments 178-186 and 188-211, wherein said polynucleotide comprises the sequence set forth in SEQ ID NO: 156.

213. The polynucleotide of any of embodiments 178-186 and 188-210, wherein said polynucleotide comprises the sequence set forth in SEQ ID NO: 157 or a sequence that exhibits at least 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 157.

214. The polynucleotide of any of embodiments 178-186, 188-210 and 213, wherein said polynucleotide comprises the sequence set forth in SEQ ID NO: 157.

215. The polynucleotide of any of embodiments 178-214, further comprising a CD33 signal sequence, a GM-CSF signal sequence, a CD8 signal sequence or an Ig kappa signal sequence.

216. The polynucleotide of any of embodiments 178-214, further comprising a CD33 signal sequence.

217. The polynucleotide of embodiment 215 or embodiment 216, wherein the CD33 signal sequence is set forth in SEQ ID NO:190 or a nucleic acid sequence having at least at or about 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO:190.

218. A vector, comprising the polynucleotide of any of embodiments 178-217.

219. The vector of embodiment 218, wherein the vector is a viral vector.

220. The vector of embodiment 219, wherein the viral vector is a retroviral vector or a lentiviral vector.

221. A cell comprising the anti-ROR1 antibody or antigen-binding fragment thereof of any of embodiments 1-53 and 244-265, the conjugate of embodiment 55 or embodiment 56, the single chain cell surface protein of embodiment 54, the anti-ROR1 chimeric antigen receptor of any of embodiments 57-177 and 266-297, the polynucleotide of any of embodiments 178-217, or the vector of any of 218-220.

222. The cell of embodiment 221, that is a lymphocyte.

223. The cell of embodiment 222, that is an NK cell or a T cell.

224. The cell of embodiment 222 or embodiment 223, wherein the cell is a T cell and the T cell is a CD4+ or a CD8+ T cell.

225. The cell of any of embodiments 221-224, wherein the cell is a primary cell obtained from a subject.

226. The cell of any of embodiments 221-225, wherein, among a plurality of the cells, less than at or about 10%, at or about 9%, at or about 8%, at or about 7%, at or about 5%, at or about 4%, at or about 3%, at or about 2% or at or about 1% of the cells in the plurality comprise an anti-ROR1 chimeric antigen receptor that exhibits tonic signaling and/or antigen independent activity or signaling.

227. A composition comprising the anti-ROR1 antibody or antigen-binding fragment thereof of any of embodiments 1-53 and 244-265, the conjugate of embodiment 55 or embodiment 56, the single chain cell surface protein of embodiment 54 or the anti-ROR1 chimeric antigen receptor of any of embodiments 57-177 and 266-297.

228. A composition comprising the cell of any of embodiments 221-226.

229. The composition of embodiment 227 or embodiment 228, further comprising a pharmaceutically acceptable excipient.

230. The composition of embodiment 228 or embodiment 229, wherein the composition comprises CD4+ and CD8+ T cells and the ratio of CD4+ to CD8+ T cells is from at or about 1:3 to 3:1, optionally at or about 1:2 to 2:1, optionally at or about 1:1.

231. The composition of any of embodiments 227-230, wherein, among a plurality of the cells, less than at or about 10%, at or about 9%, at or about 8%, at or about 7%, at or about 5%, at or about 4%, at or about 3%, at or about 2% or at or about 1% of the cells in the plurality comprise an anti-ROR1 chimeric antigen receptor that exhibits tonic signaling and/or antigen independent activity or signaling.

232. A method of treatment, comprising administering the composition of any of embodiments 227-231 to a subject having a disease or disorder associated with ROR1.

233. A method of treatment, comprising administering the anti-ROR1 antibody or antigen-binding fragment thereof of any of embodiments 1-53 and 244-265, the conjugate of embodiment 55 or embodiment 56, the single chain cell surface protein of embodiment 54, the anti-ROR1 chimeric antigen receptor of any of embodiments 57-177 and 266-297, the polynucleotide of any of embodiments 178-217, the vector of any of 218-220 or the cell of any of embodiments 221-226 to a subject having a disease or disorder associated with ROR1.

234. A composition of any of embodiments 227-231 for use in treating a disease or disorder associated with ROR1.

235. Use of a composition of any of embodiments 227-231 for the manufacture of a medicament for treating a disease or disorder associated with ROR1.

236. Use of a composition of any of embodiments 227-231 for the treatment of a disease or disorder associated with ROR1.

237. The method, the composition for use or the use of any of embodiments 232-236 and 298-311, wherein the disease or disorder associated with ROR1 is associated with ROR1 expression.

238. The method, the composition for use or the use of any of embodiments 232-237 and 298-311, wherein the disease or disorder associated with ROR1 is a B cell-related disorder.

239. The method, the composition for use or the use of any of embodiments 232-238 and 298-311, wherein the disease or disorder associated with ROR1 is a cancer.

240. The method, the composition for use or the use of embodiment 239, wherein the cancer is a ROR1-expressing cancer.

241. The method, the composition for use or the use of embodiment 240, wherein the ROR1-expressing cancer is selected from the group consisting of B cell leukemia, lymphoma, B cell chronic lymphocytic leukemia (CLL), acute myeloid leukemia (AML), acute lymphocytic leukemia (ALL), Burkitt's Lymphoma, mantle cell lymphoma (MCL), non-small cell lung cancer (NSCLC), neuroblastoma, renal cell carcinoma, colon cancer, colorectal cancer, breast cancer, epithelial squamous cell cancer, melanoma, myeloma, stomach cancer, brain cancer, lung cancer, pancreatic cancer, cervical cancer, ovarian cancer, liver cancer, bladder cancer, prostate cancer, testicular cancer, thyroid cancer, uterine cancer, adrenal cancer and head and neck cancer.

242. A kit comprising the anti-ROR1 antibody or antigen-binding fragment thereof of any of embodiments 1-53 and 244-265, the single chain cell surface protein of embodiment 54, the conjugate of embodiment 55 or embodiment 56, the anti-ROR1 chimeric antigen receptor of any of embodiments 57-177 and 266-297, the cell of embodiment 221-226 or the composition of any of embodiments 227-231, and instructions for use, optionally wherein the instructions are for administering the anti-ROR1 antibody or antigen-binding fragment thereof, the single chain cell surface protein, the conjugate, the anti-ROR1 chimeric antigen receptor, the cell or the composition, optionally in accord with the method, the composition for use or the use of any of embodiments 234-241 and 298-311.

243. An article of manufacture comprising the anti-ROR1 antibody or antigen-binding fragment thereof of any of embodiments 1-53 and 244-265, the single chain cell surface protein of embodiment 54, the conjugate of embodiment 55 or embodiment 56, the anti-ROR1 chimeric antigen receptor of any of embodiments 57-177 and 266-297, the cell of embodiment 221-226, the composition of any of embodiments 227-231 or the kit of embodiment 242.

244. The anti-ROR1 antibody or antigen-binding fragment thereof of any of embodiments 1-53, wherein the V_{H} region comprises a CDR-H1, a CDR-H2 and a CDR-H3 comprising the sequence set forth in SEQ ID NOS:52, 56 and 58, respectively, and the V_{L} region comprises a CDR-L1, a CDR-L2 and a CDR-L3 comprising the sequence set forth in SEQ ID NOS:60, 62 and 64, respectively.

245. The anti-ROR1 antibody or antigen-binding fragment thereof of any of embodiments 1-53, wherein the V_{H} region comprises a CDR-H1, a CDR-H2 and a CDR-H3 comprising the sequence set forth in SEQ ID NOS:52, 97 and 99, respectively, and the V_{L} region comprises a CDR-L1, a CDR-L2 and a CDR-L3 comprising the sequence set forth in SEQ ID NOS:60, 62 and 64, respectively.

246. The anti-ROR1 antibody or antigen-binding fragment thereof of any of embodiments 1-53, wherein the V_{H} region comprises a CDR-H1, a CDR-H2 and a CDR-H3 contained within SEQ ID NO:103, and the V_{L} region comprises a CDR-L1, a CDR-L2 and a CDR-L3 contained within SEQ ID: NO 106.

247. The anti-ROR1 antibody or antigen-binding fragment thereof of any of embodiments 1-53, wherein the V_{H} region comprises a CDR-H1, a CDR-H2 and a CDR-H3 contained within SEQ ID NO:130, and the V_{L} region comprises a CDR-L1, a CDR-L2 and a CDR-L3 contained within SEQ ID: NO 106.

248. The anti-ROR1 antibody or antigen-binding fragment thereof of any of embodiments 1-53, wherein the V_{H} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO:103, and the V_{L} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO:106.

249. The anti-ROR1 antibody or antigen-binding fragment thereof of any of embodiments 1-53, wherein the V_{H} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO:130, and the V_{L} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO:106.

250. The anti-ROR1 antibody or antigen-binding fragment thereof of any of embodiments 1-53, wherein the V_{H} region and the V_{L} region are or comprise the sequence set forth in SEQ ID NOS: 103 and 106, respectively.

251. The anti-ROR1 antibody or antigen-binding fragment thereof of any of embodiments 1-53, wherein the V_{H} region and the V_{L} region are or comprise the sequence set forth in SEQ ID NOS:130 and 106, respectively.

252. The anti-ROR1 antibody or antigen-binding fragment thereof of any of embodiments 1-53 and 244-251, wherein the antibody is a full length antibody.

253. The anti-ROR1 antibody or antigen-binding fragment thereof of any of embodiments 1-53 and 244-251, wherein the antibody is an antigen-binding fragment.

254. The anti-ROR1 antibody or antigen-binding fragment thereof of any of embodiments 1-53 and 244-253, wherein said anti-ROR1 antibody or antigen-binding fragment thereof is recombinant.

255. The anti-ROR1 antibody or antigen-binding fragment thereof of any of embodiments 1-53 and 244-254, wherein the V_{H} region and the V_{L} region is human or is from a human protein.

256. The anti-ROR1 antibody or antigen-binding fragment thereof of any of embodiments 1-53 and 244-255, wherein the scFv is or comprises the sequence set forth in SEQ ID NO: 109.

257. The anti-ROR1 antibody or antigen-binding fragment thereof of any of embodiments 1-53 and 244-255, wherein the scFv is or comprises the sequence set forth in SEQ ID NO: 134.

258. The anti-ROR1 antibody or antigen-binding fragment thereof of any of embodiments 1-53 and 244-257, wherein said anti-ROR1 antibody or antigen-binding fragment thereof specifically binds to an epitope consisting of the sequence set forth in SEQ ID NO:199 or an epitope present within the sequence set forth in SEQ ID NO:199.

259. The anti-ROR1 antibody or antigen-binding fragment thereof of any of embodiments 1-53 and 244-257, wherein the antibody or antigen-binding fragment thereof further binds to one or more epitopes consisting of a sequence selected from among any one of SEQ ID NOS: 200-214 or an epitope present within a sequence selected from among any one of SEQ ID NOS: 200-214.

260. The anti-ROR1 antibody or antigen-binding fragment thereof of any of embodiments 1-53 and 244-259, wherein the antibody or antigen-binding fragment thereof binds to human ROR1 protein with an equilibrium dissociation constant (K_{D}) of from about 1×10¹¹ M to about 1×10⁻⁷ M.

261. The anti-ROR1 antibody or antigen-binding fragment thereof of any of embodiments 1-53 and 244-260, wherein the antibody or antigen-binding fragment thereof binds to human ROR1 protein with an equilibrium dissociation constant (K_{D}) of from about 1×10⁸ M to about 1×10⁻⁷ M.

262. The anti-ROR1 antibody or antigen-binding fragment thereof of any of embodiments 1-53 and 244-260, wherein the antibody or antigen-binding fragment thereof binds to human ROR1 protein with an equilibrium dissociation constant (K_{D}) of from about 5×10⁻¹¹ M to about 1×10⁻¹⁰ M.

263. The anti-ROR1 antibody or antigen-binding fragment thereof of any of embodiments 1-53 and 244-262, wherein the antibody or antigen-binding fragment thereof binds to human ROR1 protein with a dissociation rate constant (kₐ or k_{off}) of from about 1×10⁻⁵ 1/s to about 1×10⁻² 1/s.

264. The anti-ROR1 antibody or antigen-binding fragment thereof of any of embodiments 1-53 and 244-263, wherein the antibody or antigen-binding fragment thereof binds to human ROR1 protein with a dissociation rate constant (kₐ or k_{off}) of from about 1×10⁻³ 1/s to about 1×10⁻² 1/s.

265. The anti-ROR1 antibody or antigen-binding fragment thereof of any of embodiments 1-53 and 244-263, wherein the antibody or antigen-binding fragment thereof binds to human ROR1 protein with a dissociation rate constant (kₐ or k_{off}) of from about 1×10⁻¹/s to about 1×10⁻⁴ 1/s.

266. The anti-ROR1 chimeric antigen receptor of any of embodiments 57-177, wherein the at least a portion of a hinge region comprises all or a portion of an IgG4 hinge region, optionally a human IgG4 hinge region, or a variant thereof.

267. The anti-ROR1 chimeric antigen receptor of any of embodiments 57-177, wherein the at least a portion of a hinge region comprises all or a portion of an IgG2 hinge region, optionally a human IgG2 hinge region, or a variant thereof.

268. The anti-ROR1 chimeric antigen receptor of any of embodiments 57-177, wherein the spacer is or comprises the sequence set forth in SEQ ID NO: 1.

269. The anti-ROR1 chimeric antigen receptor of any of embodiments 57-177, wherein the spacer is or comprises the sequence set forth in SEQ ID NO: 135.

270. The anti-ROR1 chimeric antigen receptor of any of embodiments 57-177, wherein the spacer comprises at least a portion of a hinge region and at least a portion of a C_{H}3 region of an immunoglobulin or a variant thereof.

271. The anti-ROR1 chimeric antigen receptor of any of embodiments 57-177 and 270, wherein the at least a portion of a CH3 region comprises all or a portion of an IgG4 CH3 and/or an IgG2 CH3, wherein the IgG4 CH3 is optionally a human IgG4 CH3 and the IgG2 CH3 is optionally a human IgG2 CH3.

272. The anti-ROR1 chimeric antigen receptor of any of embodiments 57-177, 270 and 271, wherein the spacer is at or about 111, 112, 113, 114, 115, 116, 117, 118, 119, 120, 121, 122, 123, 124 or 125 amino acids in length, or has a length between any of the foregoing.

273. The anti-ROR1 chimeric antigen receptor of any of embodiments 57-177 and 270-272, wherein the spacer is at or about 120 amino acids in length.

274. The anti-ROR1 chimeric antigen receptor of any of embodiments 57-177 and 270-273, wherein the spacer is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 3 or 138.

275. The anti-ROR1 chimeric antigen receptor of any of embodiments 57-177 and 270-274, wherein the spacer is or comprises the sequence set forth in SEQ ID NO: 3 or 138.

276. The anti-ROR1 chimeric antigen receptor of any of embodiments 57-177, wherein the spacer comprises at least a portion of a hinge region, at least a portion of a CH2 and at least a portion of a CH3 region of an immunoglobulin or a variant thereof.

277. The anti-ROR1 chimeric antigen receptor of any of embodiments 57-177 and 276, wherein the at least a portion of a CH2 region comprises all or a portion of an IgG4 CH2 and/or an IgG2 CH2, wherein the IgG4 CH2 is optionally a human IgG4 CH2 and the IgG2 CH2 is optionally a human IgG2 CH2.

278. The anti-ROR1 chimeric antigen receptor of any of embodiments 57-177, 276 and 277, wherein the spacer is at or about 130, 140, 150, 160, 170, 180, 190, 200, 210, 220, 221, 222, 223, 224, 225, 226, 227, 228, 229 or 230 amino acids in length, or has a length between any of the foregoing.

279. The anti-ROR1 chimeric antigen receptor of any of embodiments 57-177 and 276-278, wherein:
one or more of the hinge region, the CH2 region and the CH3 region comprises all or a portion of a CH2 region and all or a portion of a CH3 region from human IgG4; or
one or more of the hinge region, the CH2 region and the CH3 region is chimeric and comprises a hinge, a CH2 region and a CH3 region from human IgG4 and human IgG2; or the spacer comprises a IgG4/2 chimeric hinge region or a modified IgG4 hinge region comprising at least one amino acid replacement compared to a human IgG4 hinge; an IgG2/4 chimeric CH2 region; and an IgG4 CH3 region.

280. The anti-ROR1 chimeric antigen receptor of any of embodiments 57-177 and 276-279, wherein the spacer is or comprises an amino acid sequence having at least at or about 85%, 86%, 87%, 88%, 89%, 90%, 91%, 92%, 93%, 94%, 95%, 96%, 97%, 98% or 99% identity to SEQ ID NO:37 or 194.

281. The anti-ROR1 chimeric antigen receptor of any of embodiments 57-177 and 276-280, wherein the spacer is or comprises the sequence set forth in SEQ ID NO: 37 or 194.

282. The anti-ROR1 chimeric antigen receptor of any of embodiments 57-177 and 266-281, wherein the transmembrane region is or comprises a transmembrane domain from CD4, CD28, or CD8.

283. The anti-ROR1 chimeric antigen receptor of any of embodiments 57-177 and 266-282, wherein the transmembrane region is or comprises a transmembrane domain from CD28, optionally a human CD28.

284. The anti-ROR1 chimeric antigen receptor of any of embodiments 57-177 and 266-283, wherein the transmembrane domain is or comprises SEQ ID NO: 8 or 149 or an amino acid sequence having at least at or about 90%, 91%, 92%, 93%, 94%, 95%, 96%, 97%, 98% or 99% sequence identity to SEQ ID NO: 8 or 149.

285. The anti-ROR1 chimeric antigen receptor of any of embodiments 57-177 and 266-284, wherein the intracellular signaling region further comprises a costimulatory signaling region.

286. The anti-ROR1 chimeric antigen receptor of any of embodiments 57-177 and 266-285, wherein the anti-ROR1 chimeric antigen receptor is or comprises the sequence set forth in SEQ ID NO: 184, 185, 186, 187, 188 or 189 or a sequence that exhibits at least 85%, 86%, 87%, 88%, 89%, 90%, 91%, 92%, 93%, 94%, 95%, 96%, 97%, 98%, or 99% sequence identity to the sequence set forth in SEQ ID NO: 184, 185, 186, 187, 188 or 189.

287. The anti-ROR1 chimeric antigen receptor of any of embodiments 57-177 and 266-286, wherein the anti-ROR1 chimeric antigen receptor is or comprises the sequence set forth in SEQ ID NO: 184 or a sequence that exhibits at least 85%, 86%, 87%, 88%, 89%, 90%, 91%, 92%, 93%, 94%, 95%, 96%, 97%, 98%, or 99% sequence identity to the sequence set forth in SEQ ID NO: 184.

288. The anti-ROR1 chimeric antigen receptor of any of embodiments 57-177 and 266-286, wherein the anti-ROR1 chimeric antigen receptor is or comprises the sequence set forth in SEQ ID NO: 184.

289. The anti-ROR1 chimeric antigen receptor of any of embodiments 57-177 and 266-286, wherein the anti-ROR1 chimeric antigen receptor is or comprises the sequence set forth in SEQ ID NO: 185 or a sequence that exhibits at least 85%, 86%, 87%, 88%, 89%, 90%, 91%, 92%, 93%, 94%, 95%, 96%, 97%, 98%, or 99% sequence identity to the sequence set forth in SEQ ID NO:185.

290. The anti-ROR1 chimeric antigen receptor of any of embodiments 57-177 and 266-286, wherein the anti-ROR1 chimeric antigen receptor is or comprises the sequence set forth in SEQ ID NO: 185.

291. The anti-ROR1 chimeric antigen receptor of any of embodiments 57-177 and 266-286, wherein the anti-ROR1 chimeric antigen receptor is or comprises the sequence set forth in SEQ ID NO: 186 or a sequence that exhibits at least 85%, 86%, 87%, 88%, 89%, 90%, 91%, 92%, 93%, 94%, 95%, 96%, 97%, 98%, or 99% sequence identity to the sequence set forth in SEQ ID NO: 186.

292. The anti-ROR1 chimeric antigen receptor of any of embodiments 57-177 and 266-286, wherein the anti-ROR1 chimeric antigen receptor is or comprises the sequence set forth in SEQ ID NO: 186.

293. The anti-ROR1 chimeric antigen receptor of any of embodiments 57-177 and 266-286, wherein the anti-ROR1 chimeric antigen receptor is or comprises the sequence set forth in SEQ ID NO: 187 or a sequence that exhibits at least 85%, 86%, 87%, 88%, 89%, 90%, 91%, 92%, 93%, 94%, 95%, 96%, 97%, 98%, or 99% sequence identity to the sequence set forth in SEQ ID NO:187.

294. The anti-ROR1 chimeric antigen receptor of any of embodiments 57-177 and 266-286, wherein the anti-ROR1 chimeric antigen receptor is or comprises the sequence set forth in SEQ ID NO: 187.

295. The anti-ROR1 chimeric antigen receptor of any of embodiments 57-177 and 266-286, wherein the anti-ROR1 chimeric antigen receptor is or comprises the sequence set forth in SEQ ID NO: 188 or a sequence that exhibits at least 85%, 86%, 87%, 88%, 89%, 90%, 91%, 92%, 93%, 94%, 95%, 96%, 97%, 98%, or 99% sequence identity to the sequence set forth in SEQ ID NO: 188.

296. The anti-ROR1 chimeric antigen receptor of any of embodiments 57-177 and 266-286, wherein the anti-ROR1 chimeric antigen receptor is or comprises the sequence set forth in SEQ ID NO: 188.

297. The anti-ROR1 chimeric antigen receptor of any of embodiments 57-177 and 266-286, wherein the anti-ROR1 chimeric antigen receptor is or comprises the sequence set forth in SEQ ID NO: 189 or a sequence that exhibits at least 85%, 86%, 87%, 88%, 89%, 90%, 91%, 92%, 93%, 94%, 95%, 96%, 97%, 98%, or 99% sequence identity to the sequence set forth in SEQ ID NO:189.

298. A method of treatment, comprising administering the cell of any of embodiments 221-226 or the composition of any of embodiments 227-231 to a subject having a disease or disorder associated with ROR1.

299. The cell of any of embodiments 221-226 or the composition of any of embodiments 227-231 for use in treating a disease or disorder associated with ROR1.

300. Use of the cell of any of embodiments 221-226 or the composition of any of embodiments 227-231 for the manufacture of a medicament for treating a disease or disorder associated with ROR1.

301. Use of the cell of any of embodiments 221-226 or the composition of any of embodiments 227-231 for the treatment of a disease or disorder associated with ROR1.

302. The method, the cell, composition, antibody or antigen-binding fragment thereof, single chain cell surface protein, conjugate, chimeric antigen receptor, polynucleotide or vector for use or the use of any of embodiments 232-241 and 298-302, wherein the disease or disorder associated with ROR1 is a cancer.

303. The method, the cell, composition, antibody or antigen-binding fragment thereof, single chain cell surface protein, conjugate, chimeric antigen receptor, polynucleotide or vector for use or the use of embodiment 302, wherein the cancer is a ROR1-expressing cancer.

304. The method, the cell, composition, antibody or antigen-binding fragment thereof, single chain cell surface protein, conjugate, chimeric antigen receptor, polynucleotide or vector for use or the use of any of embodiments 232-241 and 298-303, wherein the cancer is associated with a ROR1-expressing solid tumor or a ROR1-expressing hematologic malignancy.

305. The method, the cell, composition, antibody or antigen-binding fragment thereof, single chain cell surface protein, conjugate, chimeric antigen receptor, polynucleotide or vector for use or the use of any of embodiments 232-241 and 298-304, wherein the cancer is associated with a ROR1-expressing solid tumor.

306. The method, the cell, composition, antibody or antigen-binding fragment thereof, single chain cell surface protein, conjugate, chimeric antigen receptor, polynucleotide or vector for use or the use of embodiment 304 or embodiment 305, wherein the cancer associated with a solid tumor is selected from the group consisting of neuroblastoma, renal cell carcinoma, colon cancer, colorectal cancer, breast cancer, epithelial squamous cell cancer, melanoma, myeloma, stomach cancer, brain cancer, lung cancer, pancreatic cancer, cervical cancer, ovarian cancer, liver cancer, bladder cancer, prostate cancer, testicular cancer, thyroid cancer, uterine cancer, adrenal cancer and head and neck cancer.

307. The method, the cell, composition, antibody or antigen-binding fragment thereof, single chain cell surface protein, conjugate, chimeric antigen receptor, polynucleotide or vector for use or the use of embodiment 306, wherein the lung cancer is a non-small cell lung cancer (NSCLC), lung adenocarcinoma, adenocarcinoma, squamous cell carcinoma, small cell carcinoma, and atypical carcinoid.

308. The method, the cell, composition, antibody or antigen-binding fragment thereof, single chain cell surface protein, conjugate, chimeric antigen receptor, polynucleotide or vector for use or the use of embodiment 307, wherein the lung cancer is a NSCLC.

309. The method, the cell, composition, antibody or antigen-binding fragment thereof, single chain cell surface protein, conjugate, chimeric antigen receptor, polynucleotide or vector for use or the use of embodiment 306, wherein the breast cancer is a triple negative breast cancer (TNBC).

310. The method, the cell, composition, antibody or antigen-binding fragment thereof, single chain cell surface protein, conjugate, chimeric antigen receptor, polynucleotide or vector for use or the use of any of embodiments 232-241 and 298-304, wherein the cancer is associated with a ROR1-expressing hematologic malignancy.

311. The method, the cell, composition, antibody or antigen-binding fragment thereof, single chain cell surface protein, conjugate, chimeric antigen receptor, polynucleotide or vector for use or the use of embodiment 310, wherein the hematologic malignancy is selected from the group consisting of B cell leukemia, lymphoma, B cell chronic lymphocytic leukemia (CLL), acute myeloid leukemia (AML), acute lymphocytic leukemia (ALL), Burkitt's Lymphoma or mantle cell lymphoma (MCL).

### VIII. Examples

The following examples are included for illustrative purposes only and are not intended to limit the scope of the invention.

### Example 1: Generation and Assessment of Anti-ROR1 Antibodies

Exemplary anti-receptor tyrosine kinase-like orphan receptor 1 (ROR1) antibodies that specifically bind to ROR1-expressing cells were generated by immunization of genetically engineered mice and assessed.

### A. Antibody Generation

Mice that were genetically modified to produce antibodies containing fully human antibody variable regions (Trianni, Inc., San Francisco, CA) were immunized with recombinant extracellular domain (ECD) of human ROR1 fused to a histidine tag. The spleen, lymph nodes and bone marrow were harvested, and ROR1 specific memory B cells and plasma cells were enriched by immunoaffinity-based selection.

Cell populations from the enriched fractions were subject to high-throughput single-cell paired sequencing of antibody variable heavy (V_{H}) chain and variable light (V_{L}) chain. High throughput single cell antibody sequencing was performed as generally described in WO2012/048340, WO2012/048341, WO2016/044227 and WO2016/176322. The sequencing methods employed single-cell droplets, with droplet and molecular barcodes, to identify individual pairs of antibody V_{H} and V_{L} sequences at a single-cell level, for each of a large number of single cells present in a population of cells. Antibody V_{H}/V_{L} pair sequences were selected for synthesis and polynucleotides encoding each of the paired candidates were synthesized as a single chain Fv fragment (scFv), in either a V_{H} and V_{L} chain orientation (V_{H}-V_{L}) or a V_{L}-V_{H} orientation.

### B. Anti-ROR1 Chimeric Antigen Receptor (CAR) Generation

The synthesized scFv-encoding sequences were cloned into an exemplary polynucleotide construct to generate candidate polynucleotides encoding chimeric antigen receptors (CARs) containing the candidate scFvs as the antigen-binding domains. Specifically, the polynucleotide CAR constructs contained nucleic acid sequences encoding a signal peptide; a candidate anti-ROR1 scFv; a spacer from among three exemplary spacers: a spacer containing a modified IgG4/IgG2-hinge C_{H}2-C_{H}3 (SEQ ID NO:194), a spacer containing a modified IgG4 hinge-C_{H}3 (SEQ ID NO:138), or a spacer containing a modified IgG4 hinge region (SEQ ID NO:135); a human CD28 transmembrane domain; a human 4-1BB intracellular signaling region; and a human CD3-zeta intracellular signaling region. The constructs also contained a downstream T2A ribosomal skip elements (SEQ ID NO: 6) between the CAR-encoding sequences and sequences encoding a green fluorescent protein for use as a transduction marker.

Polynucleotides encoding the CARs were cloned into a lentiviral expression vector for transduction of T cells.

### C. Assessment of Candidate Anti-ROR1 CARs

Approximately 400 candidate anti-ROR1 CAR-encoding constructs were generated as described above and viral preparations containing constructs encoding the various candidate anti-ROR1 CARs were individually introduced into a Jurkat T cell line containing a Nur77 knock-in reporter (see e.g. WO 2019/089982). The Nur77 knock-in cell line contained nucleic acid sequences encoding a reporter molecule (e.g., a red fluorescent protein) knocked-in at the endogenous Nur77 locus, which is an immediate-early response gene induced by stimulation of signal from the T cell receptor and/or via molecules containing immunoreceptor tyrosine-based activation motif (ITAM). The Jurkat reporter cells were assessed for cell surface expression of the CAR, antigen-dependent and antigen-independent signaling and cross-reactivity to a related but distinct antigen ROR2.

After transduction, the reporter cells were evaluated for the expression of red fluorescent protein after co-culture with target cells, including K562 human myelogenous leukemia cells (ROR1 negative), K562 cells transduced to express a high level of ROR1, K562 cells transduced to express a low level of ROR1, and K562 cells expressing the non-specific antigen ROR2. To further assess antigen-independent activity, transduced reporter cells were cultured alone in the absence of antigen-expressing target cells. The transduced reporter cells were also assessed for GFP expression (transduction marker), and for binding of soluble recombinant ROR1-Fc (soluble human ROR1 fused to an Fc region of an IgG).

Jurkat reporter cells transduced with candidate anti-ROR1 CARs exhibited varying levels of antigen-specific activity, as indicated by reporter expression after co-culture with ROR1-expressing target cells expressing high or low levels of ROR1; varying levels of antigen-independent (tonic) signaling, as indicated by reporter expression after co-culture with K562 cells not expressing ROR1 or after culture without antigen-expressing target cells; varying levels of non-specific activity, as indicated by reporter expression after co-culture with cells expressing the non-specific antigen ROR2; and varying levels of binding by soluble ROR1-Fc. Candidate CARs that exhibited high ROR1 antigen-dependent activity, low tonic signaling, high ROR1-Fc binding and low ROR2 cross-reactivity were selected for assessment in primary human T cells.

### Example 2: Assessment of CAR Expression and Function in Primary T Cells

The expression and function of selected anti-ROR1 CARs, described in Example 1, were assessed in primary human T cells.

CD4+ and CD8+ T cells were isolated by immunoaffinity-based enrichment from leukapheresis samples of one of three human donors. Isolated CD4+ and CD8+ T cells were mixed at approximately 1:1 ratio, stimulated and transduced with lentiviral preparations encoding one of the anti-ROR1 CARs selected as described in Example 1 (designated individually one of A-Z and A1-M1) and cultivated under conditions for expansion. The three human donors varied in the percentage of T cell subsets present in the leukapheresis sample (e.g. less than 15% naïve T cells, about 40-60% naïve T cells or about 20% effector memory RA T cells (TEMRA)), representing variability in T cell subsets among disease subjects. As controls, primary T cells also were similarly stimulated and transduced with lentiviral preparations encoding an anti-CD19 CAR, a reference anti-ROR1 CAR (a rabbit-derived reference antibody R12, scFv set forth in SEQ ID NO:143) or one of two anti-ROR1 CARs obtained from a different source (designated N1, O1).

The CAR-expressing primary T cells were stimulated with plate-bound ROR1-Fc, and after 24 hours were assessed by flow cytometry for intracellular levels of IFN-γ, TNF-α and IL-2 by intracellular cytokine staining (ICS). The percentage of cells producing IFN-γ, TNF-α and IL-2, and the level of production per cell (as determined by the mean fluorescence intensity (MFI)) were determined. After stimulation, the CAR-expressing primary T cells were also stained for cell surface expression of T cell activation markers (e.g., CD25), costimulatory markers, markers indicative of certain T cell subtypes, such as memory cell subtypes, other T cell markers, e.g., CD4 and CD8, ROR1-Fc binding, and assessed for expression of the GFP transduction marker.

CAR-expressing primary T cells also were stimulated by co-culture with ROR1-expressing triple-negative human breast cancer cell line (MDA-MB-231 (ATCC® HTB-26™), at an effector:target (E:T) ratio of 1:4, and assessed for cytotoxicity and cytokine production. The MDA-MB-231 cell line was labeled with NucLight Red (NLR) to permit tracking of target cells by microscopy. Cytolytic activity was assessed by measuring the loss of viable target cells over a period of between 0 and 72 hours, as determined by changes in red fluorescent signal. The production of cytokines IFN-γ, TNF-α and IL-2 in the supernatant was also assessed.

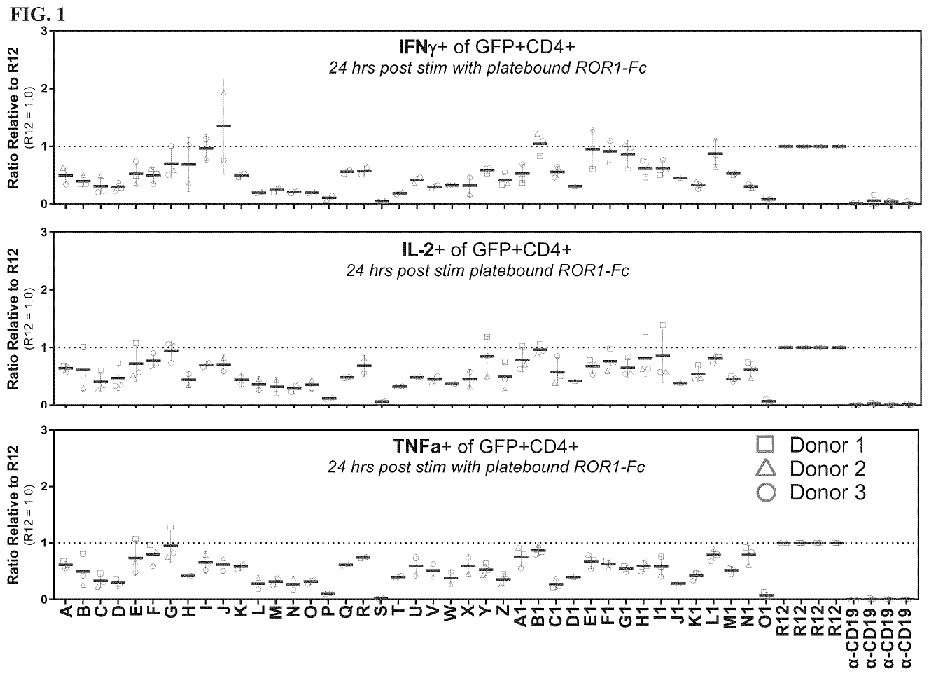

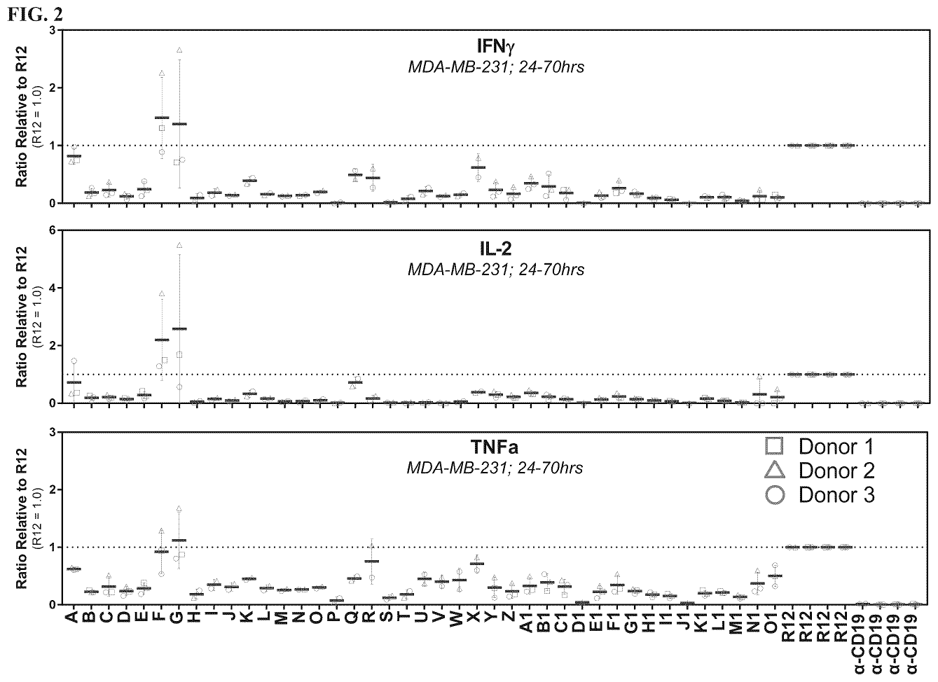

As shown in FIG. 1, certain candidate anti-ROR1 CAR-expressing T cells exhibited comparable or greater relative intracellular IFN-γ, TNF-α or IL-2 expression levels by ICS in GFP+ CD4+ T cells compared to the levels of each cytokine in cells expressing the reference anti-ROR1 (R12) CAR, after 24 hours stimulation with plate-bound recombinant ROR1-Fe. In the co-culture assay with MDA-MB-231 target cells expressing ROR1, certain selected candidates also exhibited comparable or greater IFN-γ, TNF-α or IL-2 secretion compared to cytokine secretion in cells expressing the reference anti-ROR1 (R12) CAR, as shown in FIG. 2. Fewer anti-ROR1 candidates exhibited comparable or greater cytokine secretion compared to reference anti-ROR1 (R12) CAR in the co-culture assay, compared to the number of anti-ROR1 candidates that showed comparable or greater cytokine expression levels by ICS.

These results showed varied responses by primary human T cells engineered to express the candidate anti-ROR1 CARs after stimulation by recombinant antigen or antigen-expressing target cells.

### Example 3: Polynucleotide Optimization of Selected Anti-ROR1 Chimeric Antigen Receptors (CARs)

Among the candidate anti-ROR1 CARs, six (6) candidate CARs were further selected based on results of assessment of functional activity in engineered primary human T cells from the 3 different donors as described in Example 2. The constructs encoding the selected candidate CARs were subject to codon-optimization (CO) and assessed for potential splice sites and modified in a conservative manner, such as by not altering the encoded amino acid sequence, including elimination of potential predicted splice sites (SSE) in the polynucleotide construct encoding the CAR. The constructs were further modified to replace the GFP transduction marker with a truncated receptor as a surrogate transduction marker for CAR expression.

Table E1 lists sequence identifiers (SEQ ID NO:) corresponding to amino acid (aa) sequences of the of the antigen-binding domains of the selected candidate CARs, including amino acid sequences of the corresponding variable heavy (V_{H}) chain and variable light (V_{L}) chain, and complementarity determining regions (CDRs, by Kabat numbering) of each chain. The sequence identifier (SEQ ID NO:) of the V_{H} and V_{L} chain of the reference R12 CAR also is set forth in Table E1.

The exemplary CARs contained an scFv having the V_{H}-V_{L} orientation.

TABLE E1
Sequence identifier (SEQ ID NO:) for Exemplary Candidate CARs

| | | Nucleic Acid (codon | | | | | | | | | | | | | |
|---|---|---|---|---|---|---|---|---|---|---|---|---|---|---|---|
| | | optimized and splice site | | | | | | | | | | | | | |
| | | eliminated) | | | | Amino Acid (CDR Kabat numbering) | | | | | | | | | |
| CAR | scFv # | V_{H} | V_{L} | scFv | spacer | V_{H} | CDR-H1 | CDR-H2 | CDR-H3 | VL | CDR-L1 | CDR-L2 | CDR-L3 | scFv | spacer |
| A | ROR1-2 | 120 | 123 | 126 | 136 | 121 | 82 | 86 | 88 | 124 | 90 | 92 | 94 | 127 | 135 |
| F | ROR1-1 | 111 | 114 | 117 | 136 | 112 | 67 | 71 | 73 | 115 | 75 | 77 | 79 | 118 | 135 |
| G | ROR1-1 | 111 | 114 | 117 | 139 | 112 | 67 | 71 | 73 | 115 | 75 | 77 | 79 | 118 | 138 |
| I | ROR1-3 | 102 | 105 | 108 | 139 | 103 | 52 | 56 | 58 | 106 | 60 | 62 | 64 | 109 | 138 |
| R | ROR1-2 | 120 | 123 | 126 | 139 | 121 | 82 | 86 | 88 | 124 | 90 | 92 | 94 | 127 | 138 |
| B1 | ROR1-4 | 129 | 131 | 133 | 139 | 130 | 52 | 97 | 99 | 106 | 60 | 62 | 64 | 134 | 138 |
| R12 | | | | | | | | | | | | | | 142 | |

To assess activity of CARs encoded by the polynucleotides, CD4+ and CD8+ T cells isolated by immunoaffinity-based enrichment from leukapheresis samples from two (2) human donor subjects were stimulated and transduced with lentiviral preparations encoding one of the 6 candidate CARs or the reference anti-ROR1 (R12) CAR as a control.

The cells expressing the candidate CARs were co-cultured with a ROR1-expressing human non-small cell lung cancer cell line H1975 (ATCC® CRL-5908™) labeled with NucLight Red (NLR), and cytotoxicity and cytokine production was assessed. Cytolytic activity was assessed by measuring the loss of viable target cells over a period of between 0 and 70 hours, as determined by changes in red fluorescent signal. The production of cytokines IFN-γ, TNF-α and IL-2 in the supernatant was also assessed after 70 hours of co-culture. As controls, cultures of target cells only and co-cultures of mock cells (not expressing a CAR) with the target cells were assessed.

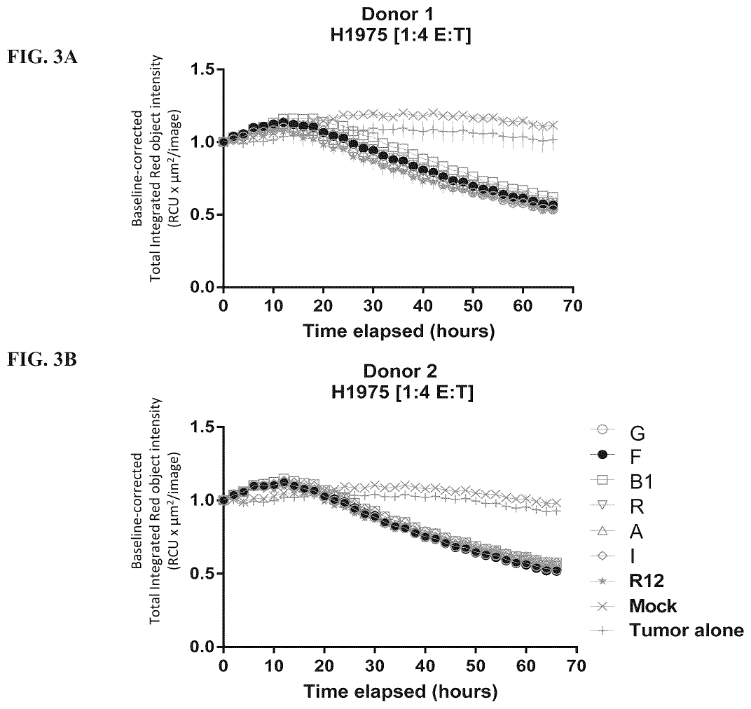

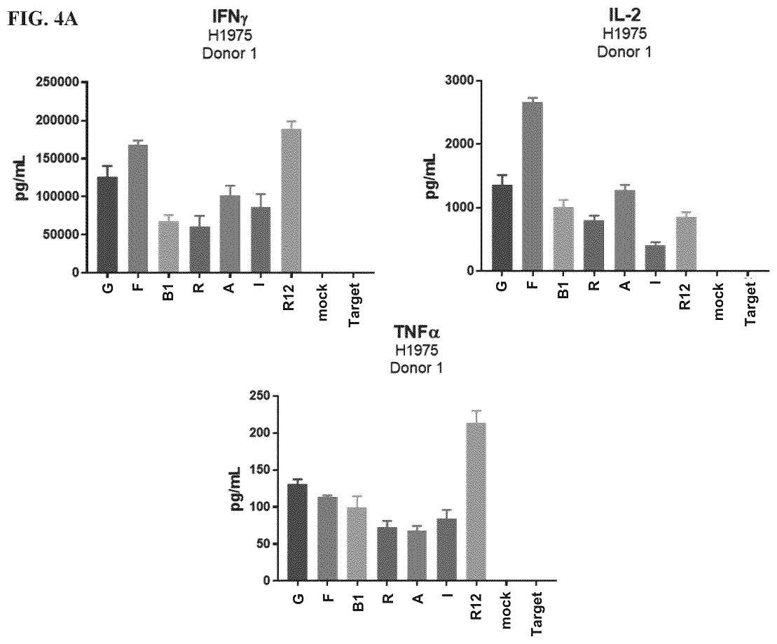

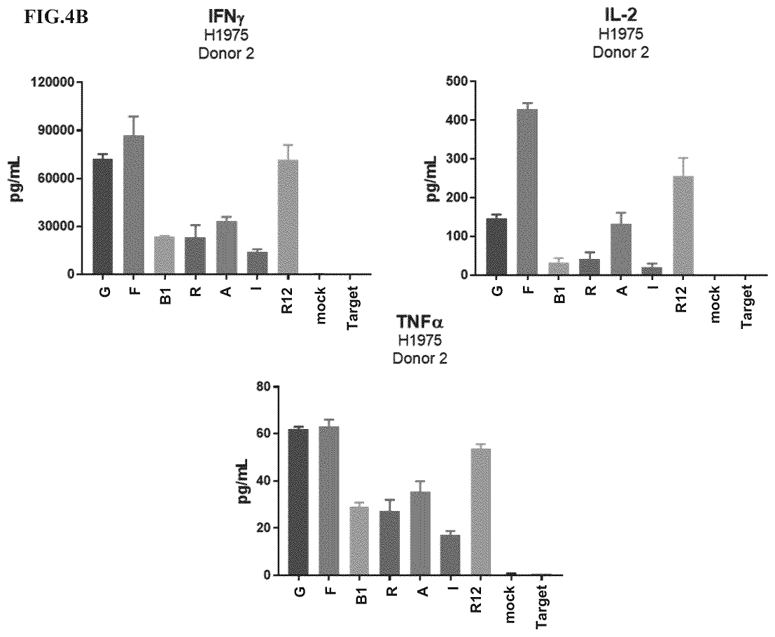

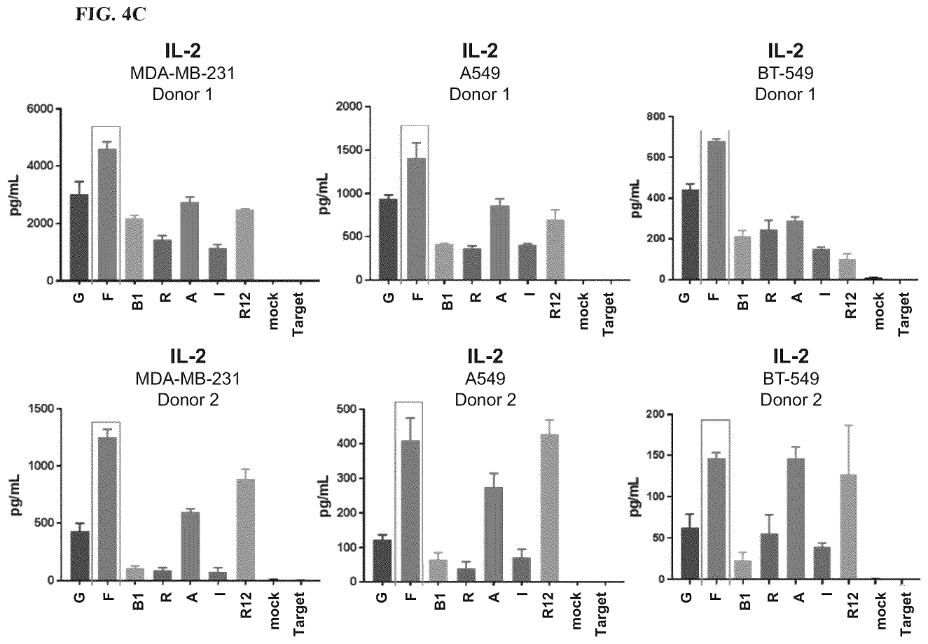

As shown in FIGS. 3A-3B, all of the candidate anti-ROR1 CARs tested showed similar in vitro cytotoxic activity against the H1975 target cells. As shown in FIGS. 4A-4B, all of the candidate anti-ROR1 CARs tested produced IFN-γ, TNF-α and IL-2 at various levels after in vitro co-culture with H1975 target cells. The results showed that after codon optimization and splice site elimination, all test constructs demonstrated cytotoxicity in co-culture with the H1975 (NSCLC) cell line. Among the tested CARs in this assay, cells expressing anti-ROR1 CAR-A, anti-ROR1 CAR-F and anti-ROR1 CAR-G generally exhibited among the highest cytokine production, which, for the various cytokines assessed, was similar to or greater than cytokine production of the cytokines from cells expressing the reference anti-ROR1 (R12) CAR. In particular, cells expressing anti-ROR1 CAR-F exhibited the highest IL-2 production compared to other candidate anti-ROR1 CARs and the reference anti-ROR1 (R12) CAR. The results were consistent with robust in vitro functional activity of the candidate CARs in co-culture with H1975 target cells.

The production of cytokines IL-2 in the supernatant was also assessed after co-culture with other ROR1 expressing target cell lines, including MDA-MB-231 human triple negative breast cancer cells, A549 human epithelial lung carcinoma cells and BT-549 human epithelial breast ductal carcinoma cells, at an E:T ratio of 1:4. As shown in FIG. 4C, all of the candidate anti-ROR1 CARs tested produced IL-2 at various levels after in vitro co-culture with MDA-MB-231, A549 and BT-549 target cells. The results showed that cells expressing anti-ROR1 CAR-F exhibited the highest IL-2 production compared to other candidate anti-ROR1 CARs and the reference anti-ROR1 (R12) CAR, when co-cultured with all of the various target cells.

### Example 4: Anti-Tumor Effect of Anti-ROR1 CAR-Expressing T Cells after Adoptive Transfer In Vivo in an Animal Model

The anti-tumor effects of exemplary engineered anti-ROR1 CAR-expressing primary human T cells were assessed by monitoring tumors following adoptive transfer of cells in tumor-bearing animal models, including a H1975 human non-small cell lung cancer xenograft model and a MDA-MB-231 human triple negative breast cancer xenograft model. The mice were administered a preparation of engineered primary human T cells generated from one of the human donors, expressing one of anti-ROR1 CARs-A, -F, -G, -I, -R and -B1 as listed in Table E1 or a reference anti-ROR1 (R12) CAR, and that had been assessed in the in vitro assays described in Example 3.

### A. H1975 Non-Small Cell Lung Cancer Model

### 1. Study 1

Eighty (80) NOD·Cg·Prkdc^{scid}IL2rg^{tm-Wjl}/SzJ (NSG) mice were each injected subcutaneously with approximately 5×10⁶ H1975 Non-Small Cell Lung Cancer cells. On day 16 following tumor engraftment, five (5) mice in each group received a single intravenous (i.v.) injection of engineered primary human T cells expressing one of anti-ROR1 CAR-A, -F, -G, -I, -R and -B1 or the reference anti-ROR1 (R12) CAR, at a dose of 1×10⁶ cells (low dose) or 3×10⁶ cells (high dose). As a control, mice were administered 3×10⁶ cells not expressing a CAR (mock) or were untreated. Survival, tumor volume and number of circulating CAR⁺ cells in the blood were assessed over approximately 65 days.

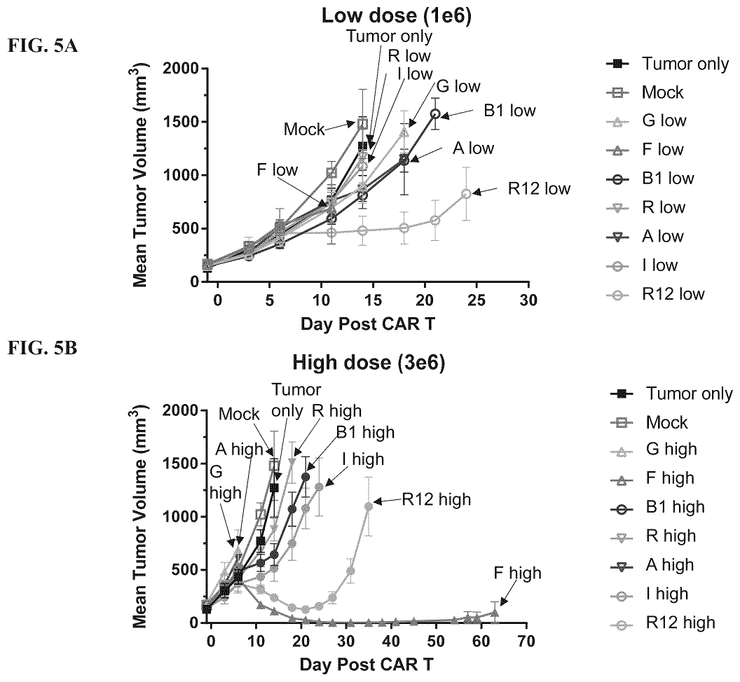

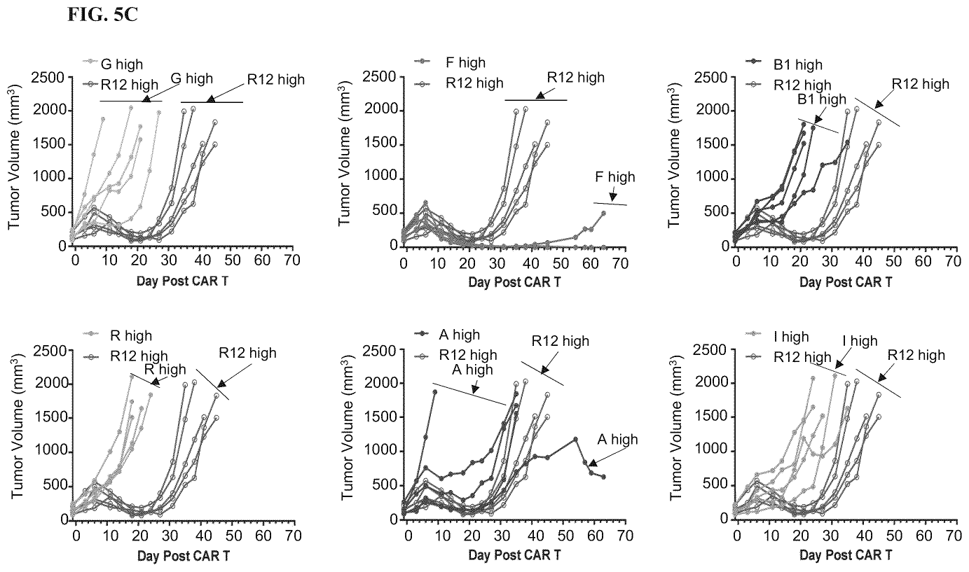

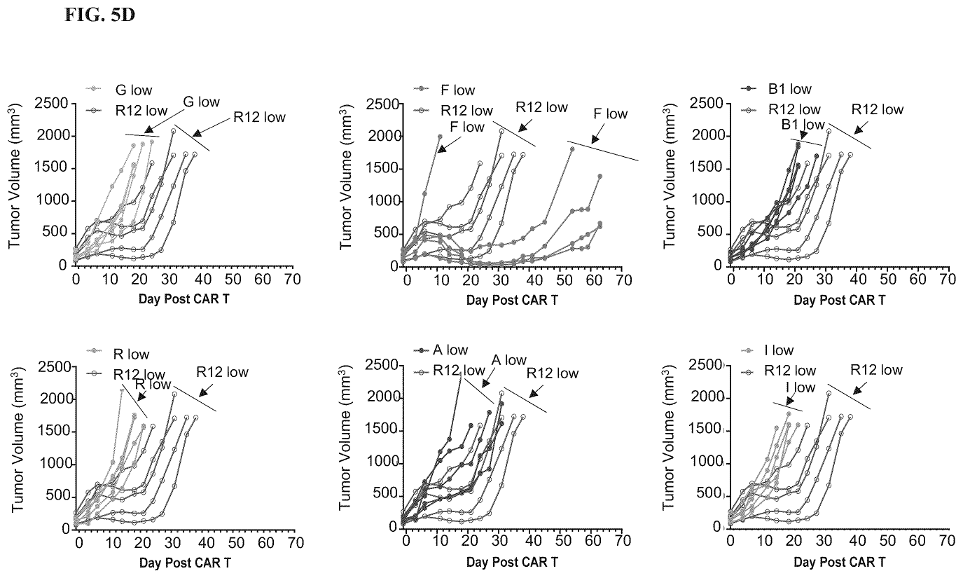

Anti-tumor activity of the adoptively transferred anti-ROR1 CAR+ T cells was monitored by determining the tumor volume every 3 to 6 days post administration. FIG. 5A (low dose) and FIG. 5B (high dose) depict mean tumor volume of all treated mice; in this depiction, tumor curves were terminated after the first mouse of a group succumbed to disease. Results from all individual mice are shown in FIG. 5C (low dose) or FIG. 5D (high dose). As shown in FIGS. 5A-5D, primary human T cells expressing the candidate anti-ROR1 CARs showed varying levels of anti-tumor activity in the H1975 mouse model. Primary human T cells expressing anti-ROR1 CAR-F exhibited the highest anti-tumor activity after a high dose administration, as shown by reduction in tumor volume, of tested anti-ROR1 CARs, including the anti-ROR (R12) reference CAR. Specifically, administration of anti-ROR1 CAR-F expressing cells resulted in mean tumor regression (below baseline) up to 52 days, compared to 6 days for the reference R12 CAR-expressing cells. 80% of H1975 tumor-bearing mice (4 of 5) treated with the high dose of anti-ROR1 CAR-F expressing cells exhibited complete and durable tumor clearance, whereas no animals treated with the high dose of the reference R12 CAR-expressing cells exhibited complete tumor clearance. At the low dose, 3 of 5 animals administered anti-ROR1 CAR-F expressing cells exhibited tumor regression below baseline up to 17 days, whereas only 1 of 5 animals administered R12 CAR-expressing cells exhibited tumor regression below baseline.

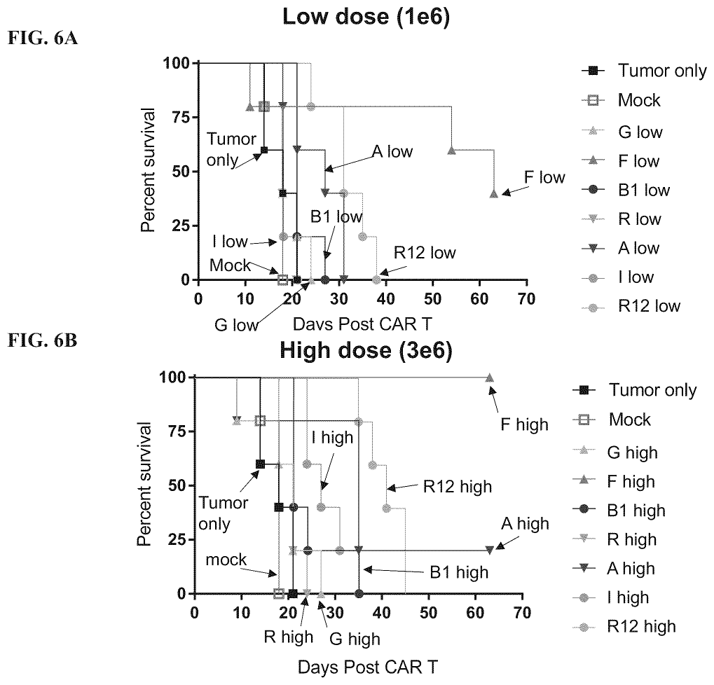

Survival of mice treated as described above was assessed. Kaplan-Meier survival curves of each of the groups are shown in FIGS. 6A-6B. As shown, administration of primary human T cells expressing the candidate CARs showed varying effects on survival of the treated mice. In this model, mice administered primary human T cells expressing anti-ROR1 CAR-F or anti-ROR1 CAR-A exhibited survival that was similar to or longer than survival of the reference anti-ROR1 (R12) CAR at one or both of the tested doses. Mice administered cells expressing anti-ROR1 CAR-F exhibited the longest survival with over 100% of mice surviving at 65 days following the initiation of administration of CAR-expressing cells at the highest dose in this model.

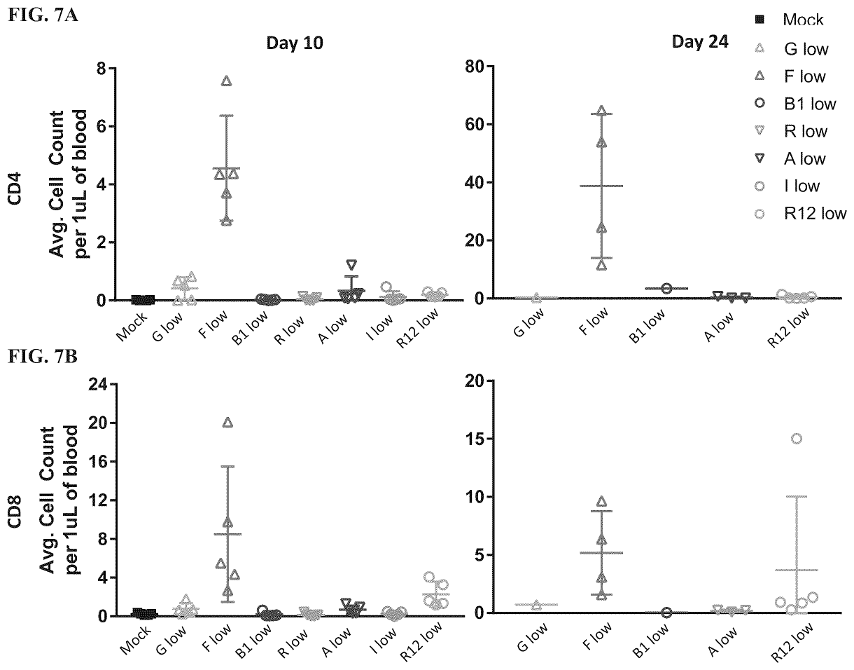

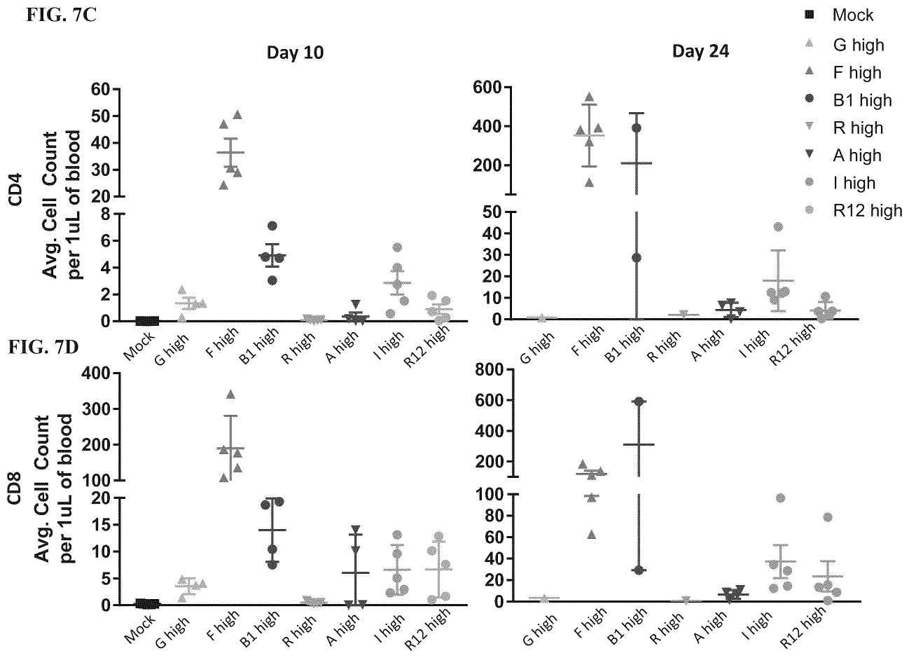

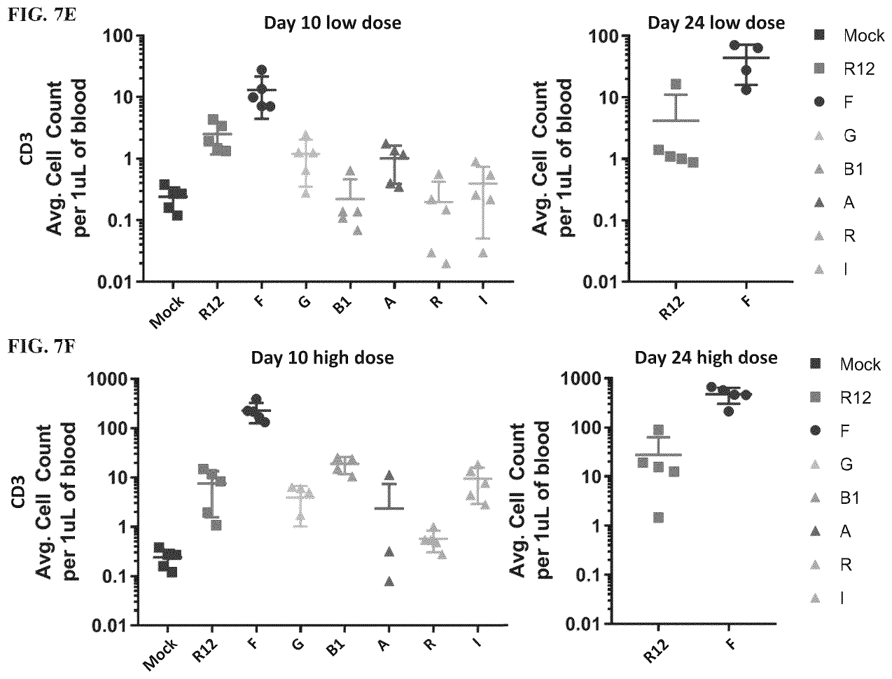

The average number of CD4+ and CD8+ CAR-expressing cells in the blood of each mouse was determined at day 10 and day 24 after administration of CAR-expressing T cells, at the low dose (FIGS. 7A and 7B) or high dose (FIGS. 7C and 7D). The average number of CD3+ CAR-expressing cells (CD45+CD3+ CAR+) in the blood of each mouse also was determined at day 10 and day 24 after administration of CAR-expressing T cells, at the low dose (FIG. 7E) or high dose (FIG. 7F). As shown in FIGS. 7A-7F, a higher average CAR+ T cell number was observed in mice administered a higher dose of CAR+ T cells. T cells expressing anti-ROR1 CAR-F exhibited robust expansion following administration at both the low and high doses in this model. At 10 days post-infusion, the mean count of CAR-F expressing cells was 226.5 and 13.1 cells/μl of blood for the high and low doses, respectively, compared to the mean count of R12 expressing cells of 7.6 and 2.5 cells/μl of blood for the high and low doses, respectively. At 24 days post-infusion, the mean count of CAR-F expressing cells was 471.8 and 44.0 cells/μl of blood at the high and low doses, respectively, compared to the count of R12 expressing cells of 27.6 and 4.2 cells/μl of blood for the high and low doses, respectively.

### 2. Study 2

In a different study using the same H1975 mouse model, sixty-four (64) mice with engrafted H1975 tumors were staged based on measurement of tumor volume and administered a single intravenous (i.v.) injection of engineered primary human T cells expressing anti-ROR1 CAR-F, anti-ROR1 CAR-A, or reference anti-ROR1 (R12) CAR, at a dose of 1×10⁶ cells (low dose) or 3×10⁶ cells (high dose), eight (8) mice in each group. As a control, mice were administered 3×10⁶ cells not expressing a CAR (mock) or were untreated. Tumor volume was measured up to approximately 70 days, and circulating CAR+ T cell number and survival were measured up to approximately 40 days.

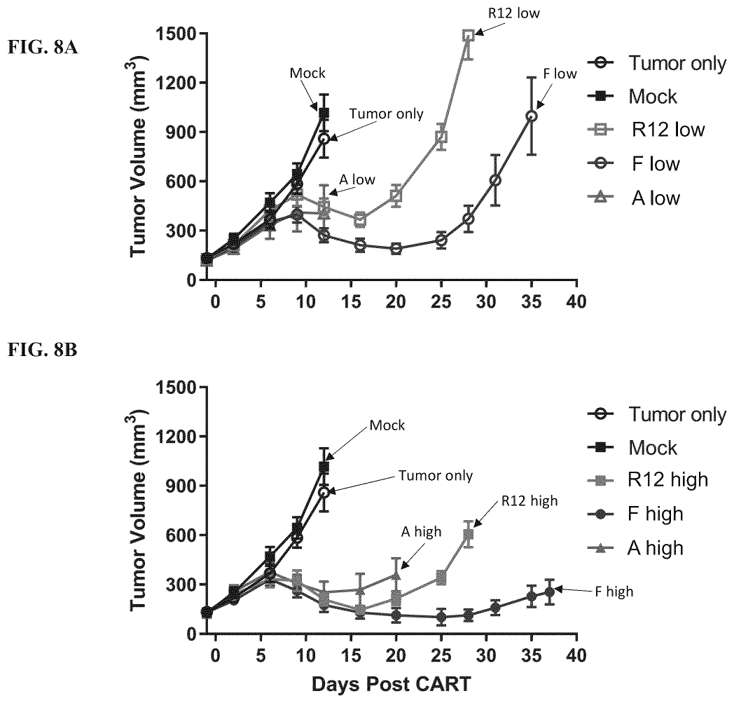

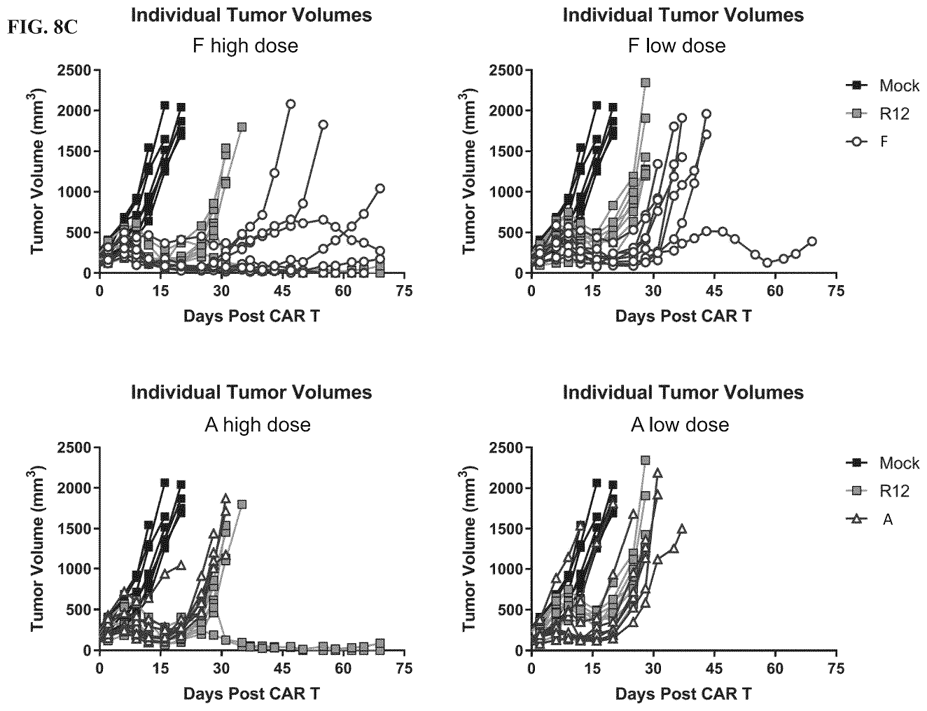

The changes in the mean tumor volume in mice that were administered anti-ROR1 CAR-F, CAR-A or the R12 reference CAR at the low or the high dose are shown in FIGS. 8A and 8B, respectively. The changes in the mean tumor volume in individual mice are shown in FIG. 8C.

Administration of anti-ROR1 CAR-F expressing cells resulted in mean tumor regression (below baseline) at 15 days, compared to administration of reference R12 CAR-expressing cells, which did not exhibit mean tumor regression below baseline (FIG. 8B). 25% of H1975 tumor-bearing mice (2 of 8) treated with the high dose of anti-ROR1 CAR-F expressing cells exhibited complete and durable tumor clearance, whereas 12.5% of mice (1 of 8) treated with 3 the high dose of the reference R12 CAR-expressing cells exhibited complete tumor clearance.

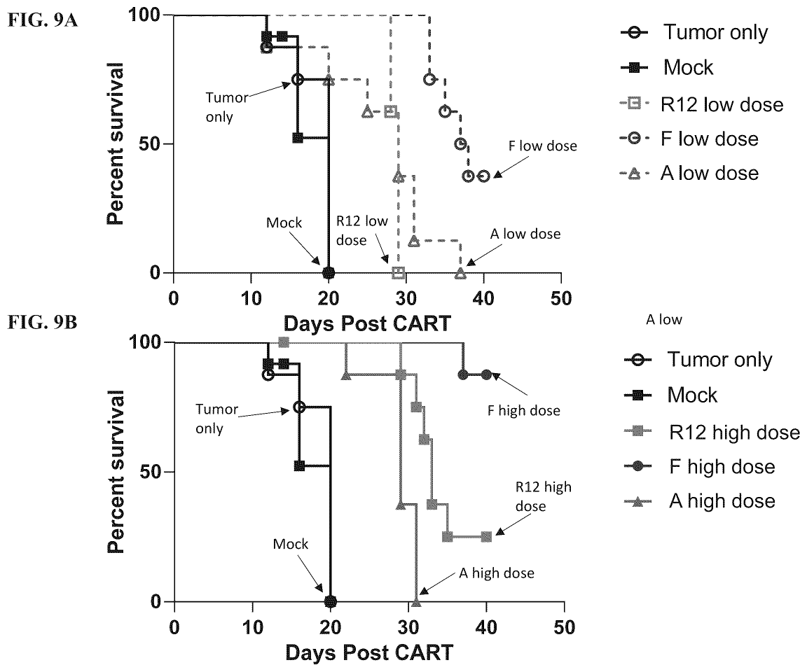

Mice were monitored for tumor growth and body condition at least twice weekly for 69 days post CAR T cell transfer and were euthanized when tumor volume exceeded 1500 mm³, when experiencing tumor ulceration, when displaying signs of severe xenogeneic graft versus host disease (xGvHD), or when otherwise moribund. The survival of mice in each group are shown in FIGS. 9A-9B. Median survival in untreated H1975 xenograft mice were left or administered mock treated cells was 20 days. Median survival of animals administered the high or low dose of R12 CAR T cells, was 33 and 29 days, respectively. Median survival in mice administered the anti-ROR1 CAR-F survived to 37.5 days for the low dose, and median survival was undetermined at the high dose, with 62.5% (5 of 8) animals remaining alive at study end.

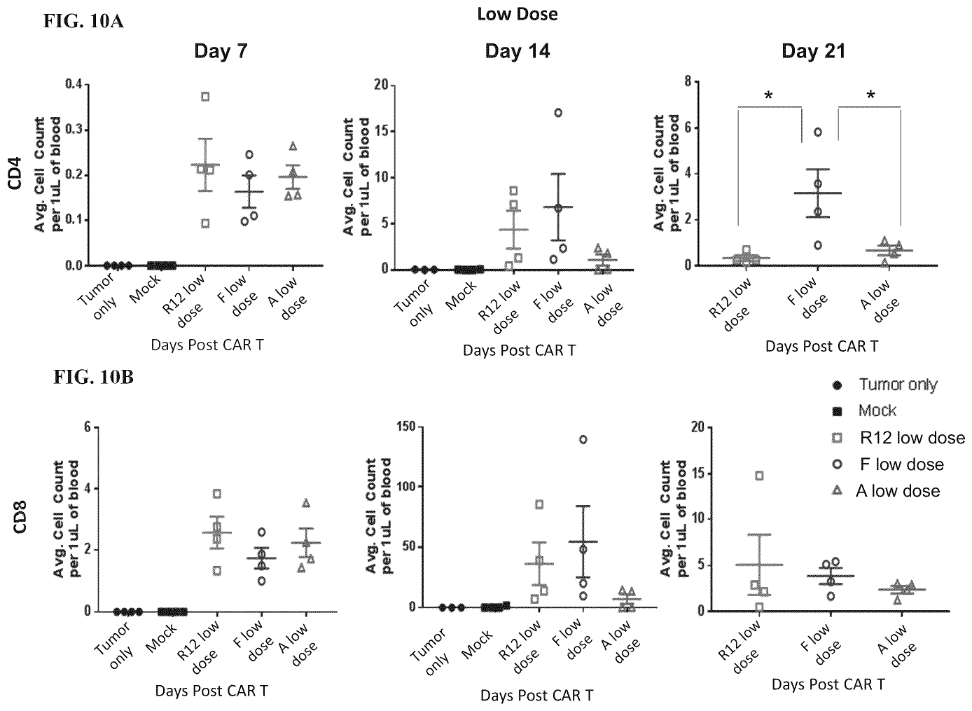

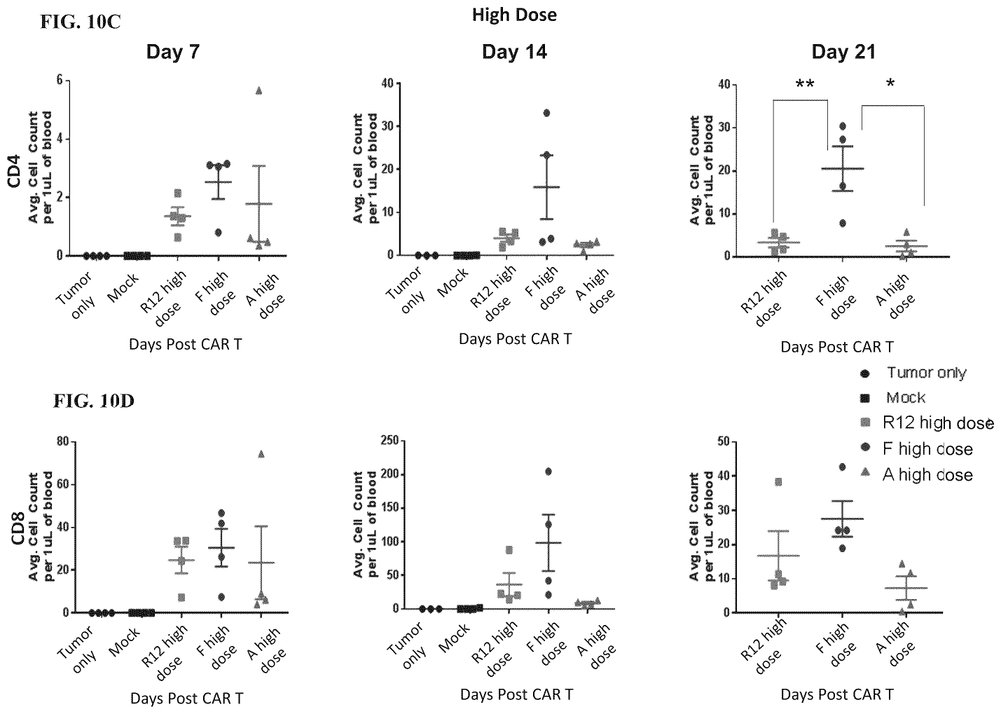

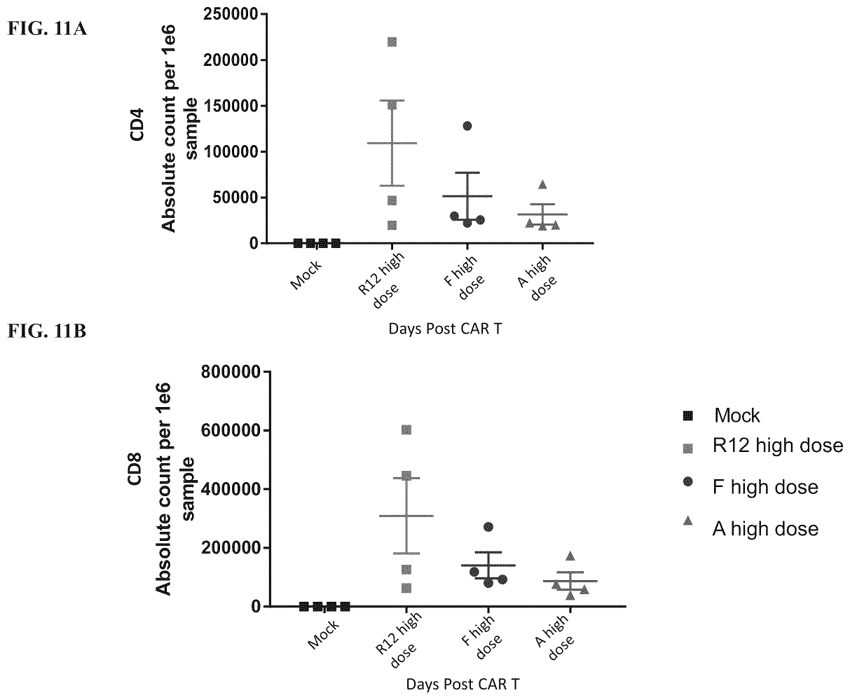

The average CD4+ and CD8+ CAR+ T cell count per microliter of blood at days 7, 14 and 21 after administration are shown in FIGS. 10A-10D. Consistent with the results in the study described above, the mean count of CAR-F expressing cells was highest compared to the meant count of the other CAR-expressing cells, particularly at day 21 and/or among CD4+ T cells (FIG. 10A and FIG. 10C), or at the high dose (FIG. 10C and FIG. 10D). At 7, 14, and 21 days post-administration of the high dose, the anti-ROR1 CAR-F expressing T cells showed mean counts of 37.6, 105.6, and 46.6 cells/μl of blood, respectively, whereas the reference R12-expressing cells showed mean counts of 28.9, 39.8, and 20.3 cells/μl of blood. At days 7, 14, and 21 post-administration of the lower dose, the anti-ROR1 CAR-F expressing cells showed mean counts of 2.2, 55.4, and 6.9 cells/μl of blood, respectively, whereas the reference R12-expressing cells showed mean counts of 3.1, 40.8, and 5.5 cells/μl of blood, respectively.

FIGS. 11A and 11B depict the number of CD4+ and CD8+ CAR+ T cells present in the tumor at 14 days after administration. The number of CAR⁺ cells in the tumor at day 14 were similar between the CARs tested.

### 3. Summary

As shown, mice with H1975 lung adenocarcinoma xenograft tumors administered anti-ROR1 CAR-F-expressing cells exhibited higher anti-tumor activity, as indicated by tumor volume reduction, longer survival, and increased number of circulating CAR+ T cells compared to mice administered the reference anti-ROR1 R12 CAR. The anti-tumor activity, survival and CAR+ T cell number was similar in mice administered anti-ROR1 CAR-A-expressing cells compared to anti-ROR1 R12 CAR-expressing cells.

### B. MDA-MB-231 Triple Negative Breast Cancer Model

Sixty-four (64) NOD·Cg·Prkdc^{scid}IL2rg^{tml Wjl}/SzJ (NSG) mice were each injected subcutaneously with 1×10⁶ MDA-MB-231 cells expressing firefly luciferase. On day 7 following tumor engraftment, eight (8) mice in each group received a single intravenous (i.v.) injection of engineered primary human T cells expressing anti-ROR1 CAR-A, anti-ROR1 CAR-F, or reference anti-ROR1 (R12) CAR, at a dose of 1×10⁶ cells (low dose) or 3×10⁶ cells (high dose). As a control, mice were administered 3×10⁶ T cells not expressing a CAR (mock) or were untreated. Bioluminescence imaging of tumor burden and flow cytometric analysis of the number of circulating CAR⁺ cells in the blood were assessed over approximately 100 days.

Anti-tumor activity of the adoptively transferred CAR-expressing T cells was monitored by bioluminescence imaging every 4 to 7 days post CAR-T cell administration. For bioluminescence imaging, mice received intraperitoneal (i.p.) injections of luciferin substrate (PerkinElmer, Waltham, MA) resuspended in PBS (approximately 150 mg/kg body weight). Mice were anesthetized and imaged essentially as described in WO2015/095895. The total flux (photon/s) was determined at each time point.

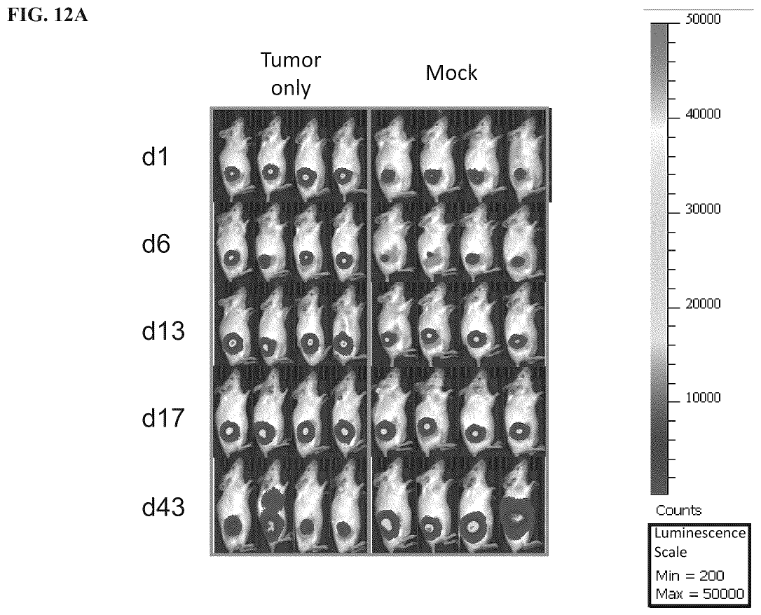

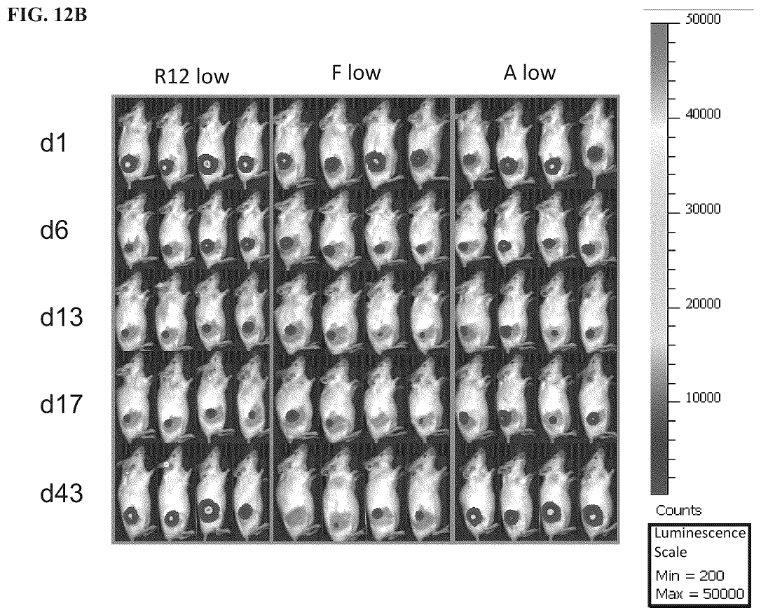

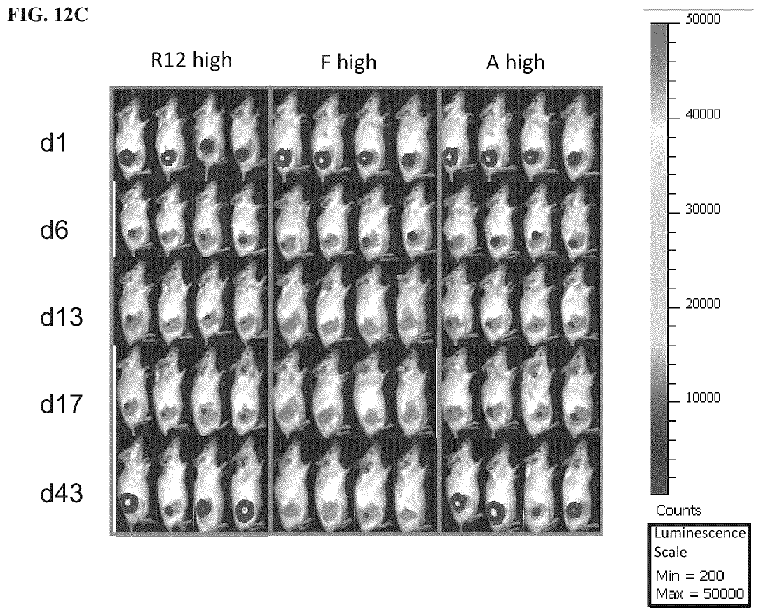

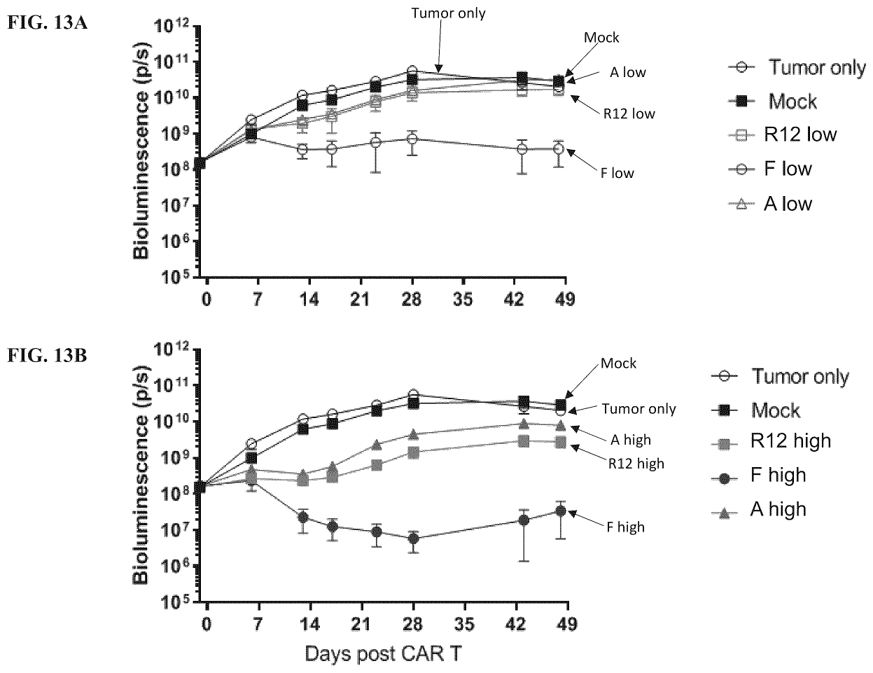

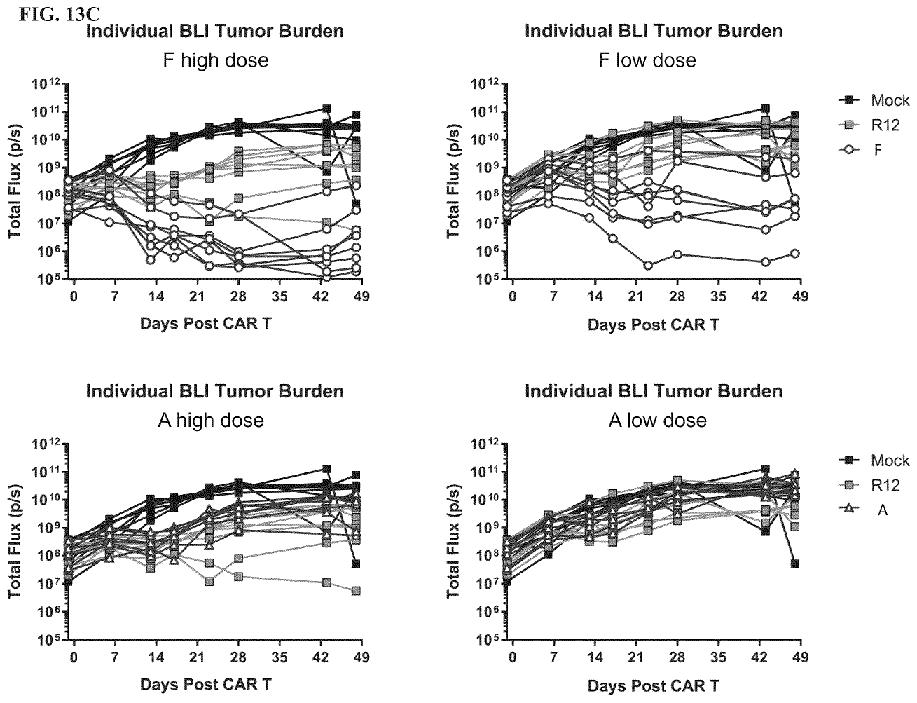

The results for bioluminescence images from treated mice and the mean measured total flux (p/s) are shown in FIGS. 12B and 13A, respectively, following administration of the low dose of CAR-expressing cells; or in FIGS. 12C and 13B, respectively, following administration of the high dose of CAR-expressing cells. FIG. 12A depicts bioluminescence images of untreated mice or mice administered mock T cells. FIG. 13C depicts the measured total flux (p/s) of individual mice.

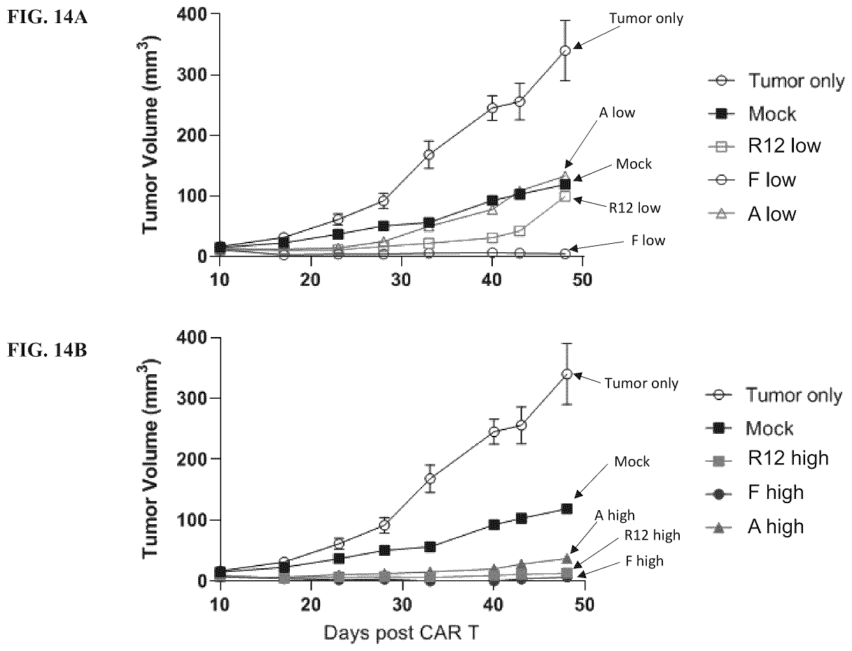

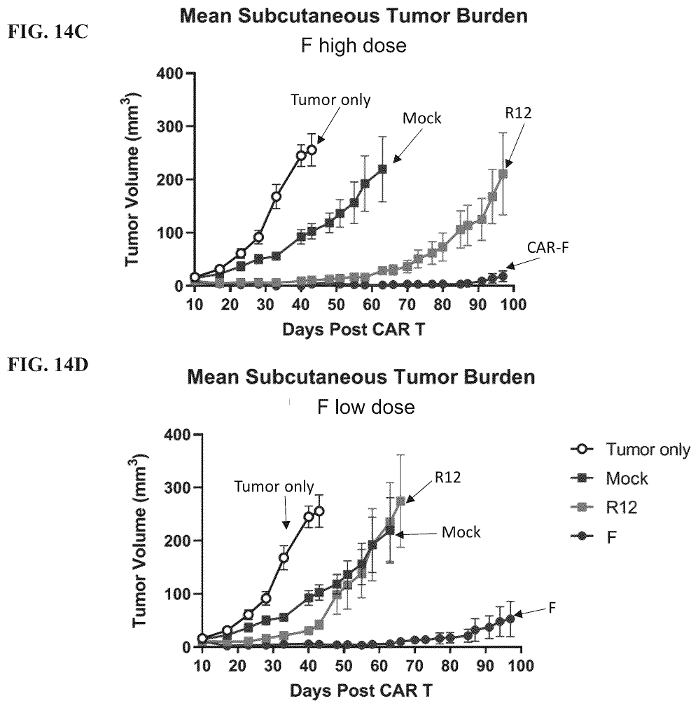

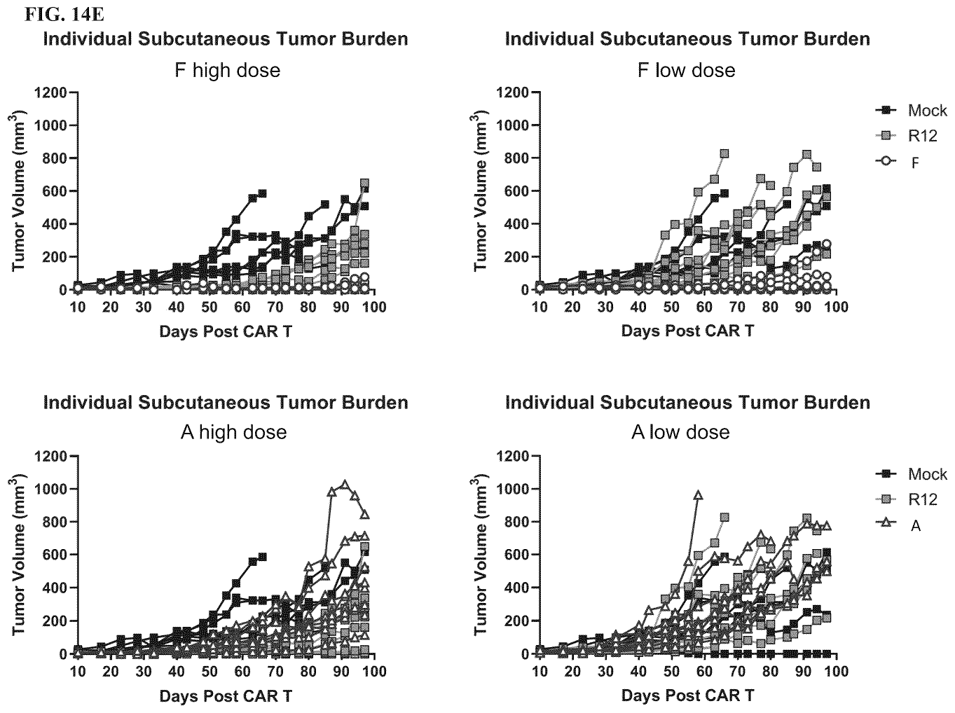

The changes in the mean tumor volume at the low dose or high dose are shown in FIGS. 14A and 14B, respectively. As shown, mice administered cells expressing anti-ROR1 CAR-F, at both the low and high doses, were observed to generally have a lower degree of bioluminescence signal, indicating a reduction in tumor growth over time and/or a lower degree of tumor growth in the treated animals, compared to mice administered cells expressing the reference anti-ROR1 (R12) CAR. Mice administered cells expressing anti-ROR1 CAR-A, at both the low and high doses, were observed to generally have similar bioluminescence signal compared to mice administered cells expressing the reference anti-ROR1 (R12) CAR.

The changes in the mean tumor volume following administration of T cells expressing anti-ROR1 CAR F and reference CAR R12, up to a further time point in the same study are shown for the high dose and low dose in FIGS. 14C and 14D, respectively. Results of tumor volume for individual treated mice at the high dose or low dose for each treated condition are shown in FIG. 14E. Administration of anti-ROR1 CAR-F expressing cells resulted in mean tumor regression (below baseline, as measured by BLI) for at least 35 days, when BLI measurements terminated. The reference R12 CAR did not exhibit mean tumor regression below baseline, at either the high dose (FIGS. 14C and 14E) or the low dose (FIGS. 14D and 14E). By subcutaneous tumor volume, 50% of mice (4 of 8) administered anti-ROR1 CAR-F expressing cells at the high dose exhibited complete and durable tumor clearance, whereas only 12.5% of mice (1 of 8) treated with the high dose of R12 CAR-expressing cells exhibited complete tumor clearance (FIGS. 14C and 14E). At the low dose, 25% (2 of 8) of animals treated with anti-ROR1 CAR-F expressing cells exhibited complete tumor clearance, whereas no animals treated with reference R12 CAR-expressing cells at the low dose resulted in complete tumor clearance (FIGS. 14D and 14E).

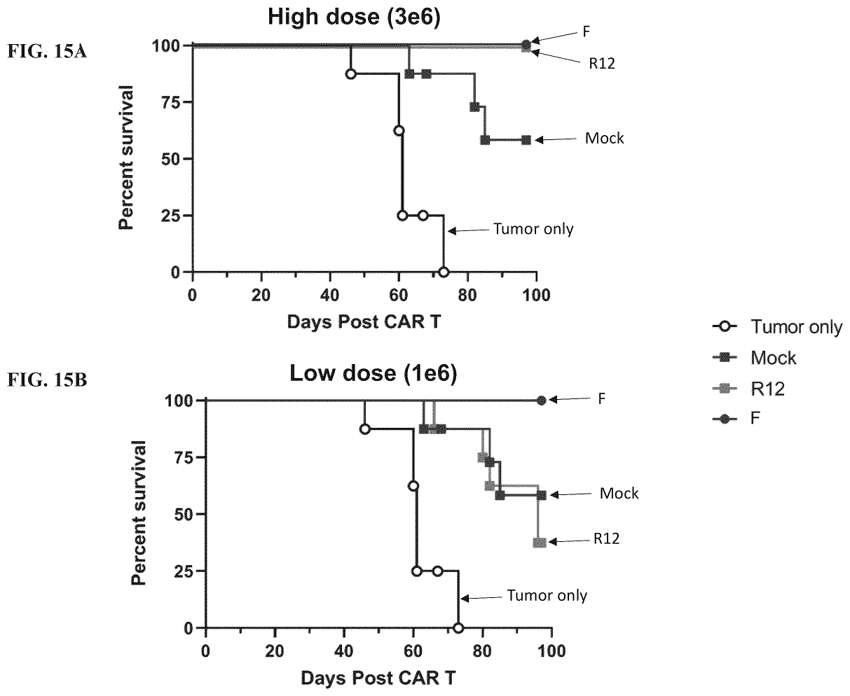

Mice were monitored for tumor growth and body condition at least twice weekly for 97 days post CAR T cell transfer and were euthanized when tumor volume exceeded 1500 mm³, when experiencing tumor ulceration, when showing signs of severe xGvHD (e.g., severe dermatitis and/or 20% weight loss attributed to xGvHD), or when otherwise moribund. Survival of mice treated as described above was assessed. Kaplan-Meier survival curves of each of the groups are shown in FIGS. 15A-15B. Median survival for untreated mice was 61 days. At the high dose, median survival was undetermined for mice administered the anti-ROR1 CAR-F expressing cells, R12 reference CAR expressing cells or mock treated cells, as 62.5% (5 of 8) of animals administered mock treated cells survived at study end, and there were no deaths in either groups administered CAR T cells (FIG. 15A). At the low dose, median survival for the mice administered the R12 CAR-expressing cells at the low dose was 96 days, with only 3 of 8 animals remaining alive at study end, whereas 100% survival (8 of 8) was observed in mice administered CAR-F expressing cells (FIG. 15B).

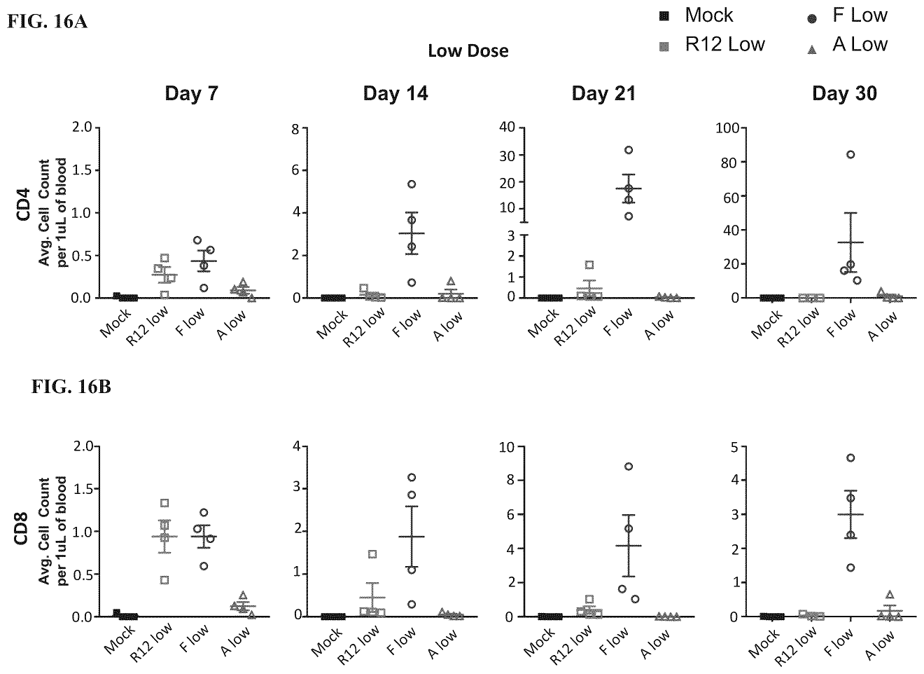

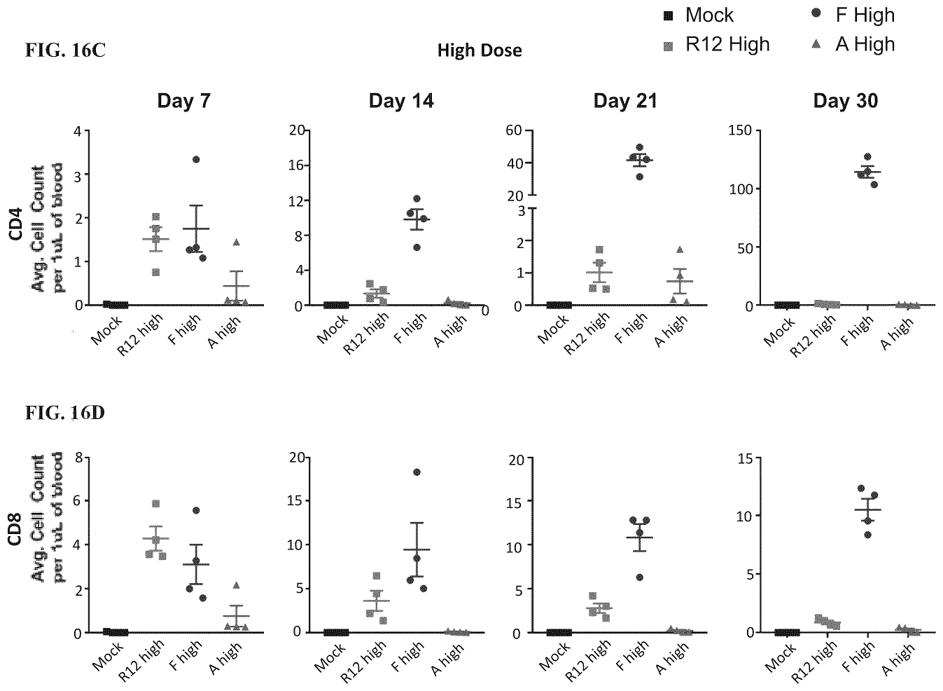

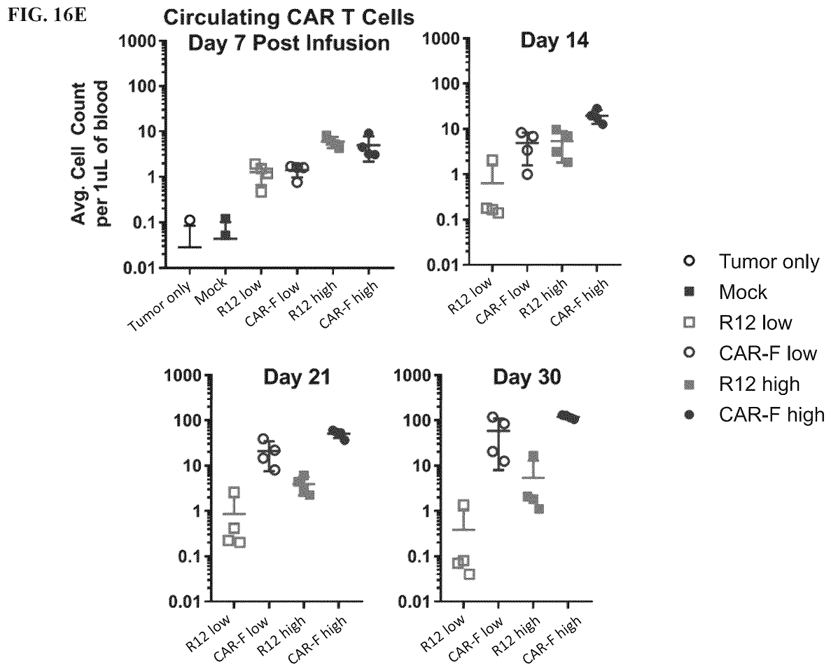

The average number of CD4+ and CD8+ CAR-expressing cells in the blood of the animal were determined at days 7, 14, 21 and 30 after administration of CAR-expressing T cells at the low dose (FIGS. 16A-16B) or high dose (FIGS. 16C-16D). The average number of CD3+ CAR-expressing cells (CD45+CD3+ CAR+) in the blood of each mouse administered cells expressing anti-ROR1 CAR-F or the R12 reference CAR also was determined at days 7, 14, 21 and 30 are shown in FIG. 16E. At the high dose, the mean count of CAR-F expressing T cells was 5.0, 19.4, 50.7 and 119.2 cells/μl of blood for days 7, 14, 21, and 30, respectively, compared to the mean count of R12 expressing T cells of 6.0, 5.4, 3.9, and 5.4 cells/μl of blood. Administration of the low dose also showed a similar expansion, with the mean count of CAR-F expressing cells of 1.4, 4.9, 20.9 and 58.5 cells/μl of blood for days 7, 14, 21, and 30, respectively, compared to the mean count of R12 expressing cells of 1.3, 0.6, 0.9, and 0.4 cells/μl of blood. As shown, cells expressing anti-ROR1 CAR-F exhibited high expansion in the MDA-MB-231 mouse model, when administered at both high and low doses. In particular, anti-ROR1 CAR F-expressing cells exhibited substantially high expansion starting at day 14 continuing to day 30 after administration.

### C. Conclusion

The results supported high anti-tumor activity and T cell expansion, and extended survival of mice administered anti-ROR1 CAR-F, using two different in vivo mouse tumor models, including the triple negative breast cancer xenograft tumor model. By most measurements, the anti-ROR1 CAR-F-expressing cells showed improvements compared to the anti-ROR1 reference R12 CAR.

### Example 5: Anti-ROR1 CAR-Expressing T Cells Generated from Human Subjects with Chronic Lymphocytic Leukemia (CLL)

Engineered anti-ROR CAR-expressing primary T cells were generated from human subjects with chronic lymphocytic leukemia (CLL) and assessed for cytotoxicity and cytokine production in vitro.

A. Generation of Anti-ROR1 CAR-expressing Cells from Subjects with CLL

Primary T cells from human subject with Chronic Lymphocytic Leukemia (CLL) were engineered to express the exemplary anti-ROR1 CAR, anti-ROR1 CAR-F, described in Example 3 above.

CD4+ and CD8+ T cells were isolated by immunoaffinity-based enrichment from leukapheresis samples of one of two human donors with CLL. Isolated CD4+ and CD8+ T cells were mixed at approximately 1:1 ratio, stimulated and transduced with lentiviral preparations encoding anti-ROR1 CAR-F as described in Example 3, an anti-CD19 CAR as a control or a reference anti-ROR1 CAR (R12), and cultivated under conditions for expansion. CD4- and CD8-depleted leukapheresis samples were assessed for CD19 and ROR1 expression levels and used as target cell population in the assays. For one of the subjects with CLL (CLL donor 1), ROR1 expression level was low, nearly as low as a K562 human chronic myelogenous leukemia (CML) cell line engineered to express ROR1 in response to the tetracycline derivative doxycycline, without addition of doxycycline (see Example 9).

### B. Cytolytic Activity and Cytokine Production

Cytolytic activity of the primary T cells from subjects with CLL expressing the exemplary anti-ROR1 CAR F were assessed.

In one experiment, target cells obtained from the subjects (CD4- and CD8-depleted leukapheresis samples) were labeled with Rapid Red cell labeling reagent to permit tracking of target cells by microscopy, and incubated with the CAR-expressing cells at an E:T ratio of 2.5:1 and 0.25:1, or cultured with mock cells or cultured alone as controls. Cytolytic activity was assessed by measuring the loss of red fluorescent signal over a period of between 0 and 60 hours, compared to controls.

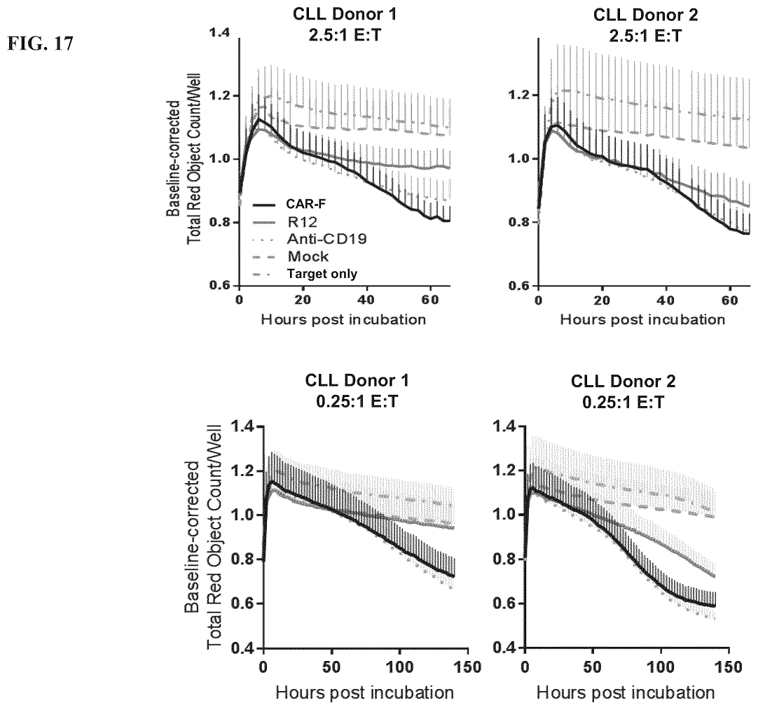

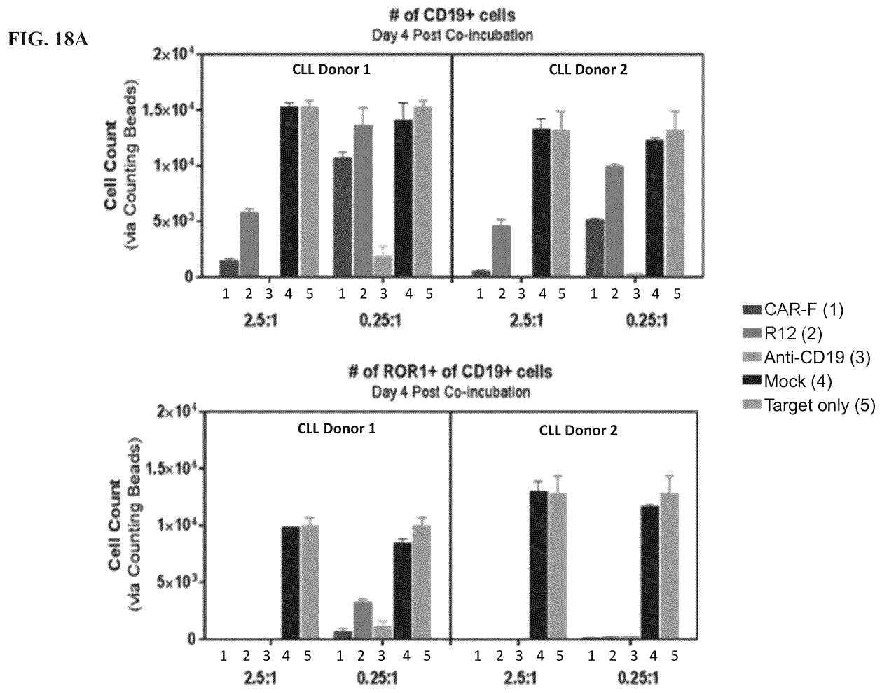

As shown in FIG. 17, for both donors and both E:T ratios, red fluorescent signal from the labeled target cells were lost more rapidly when co-cultured with CAR-expressing cells, compared to the expected loss from normal cell division observed in the target cells alone or co-culture with mock transduced cells. A greater loss of target cells was observed following co-culture of the target cells with anti-ROR1 CAR-F-expressing cells compared to anti-ROR1 R12-expressing cells, and was comparable to the loss of target cells following co-culture with anti-CD19 CAR-expressing cells.

In a different experiment, the loss of ROR1 expressing target cells was assessed by flow cytometry. For this, engineered CAR-expressing cells were labeled with CellTrace™ Violet (CTV) cell proliferation reagent, and co-cultured with target cells from the subjects with CLL at an E:T ratio of 2.5:1 and 0.25:1. On day 4, supernatants were collected to evaluate cytokine production (IFN-γ, TNF-α and IL-2) and the cells were collected to assess CTV levels by flow cytometry.

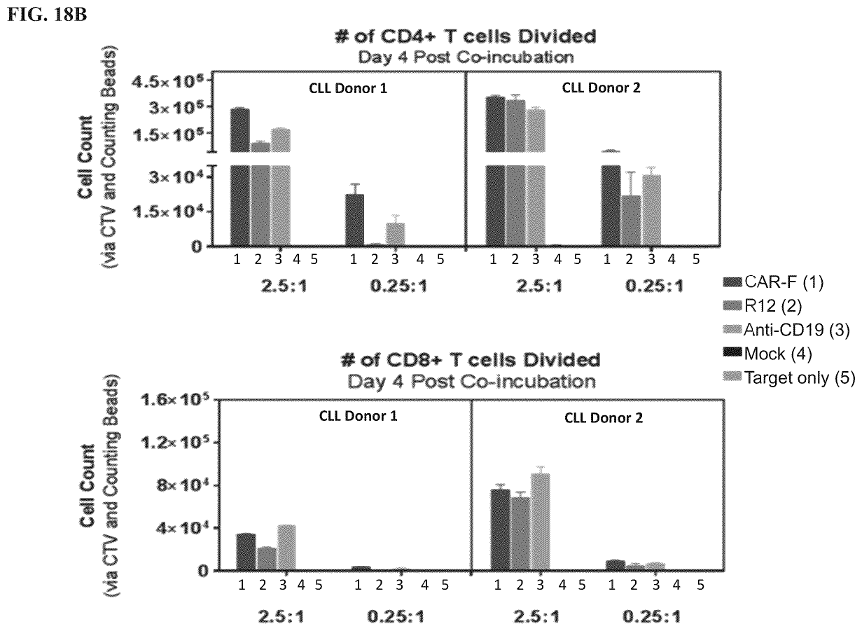

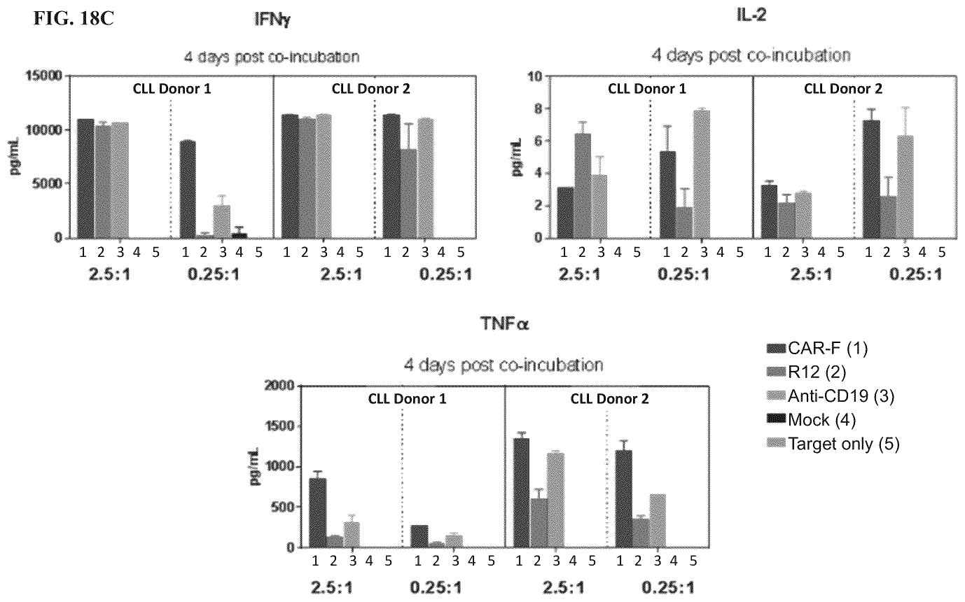

As shown in FIG. 18A, the number of ROR1+ target cells co-cultured with anti-ROR1 CAR F expressing cells generated from subjects with CLL were reduced substantially compared to target cells cultured alone or target cells co-cultured with mock transduced T cells. The reduction was greater than the reduction observed with a co-culture with R12-expressing cells, at both E:T ratios for both donors. As shown in FIGS. 18B-18C, anti-ROR1 CAR F expressing cells generated from subjects with CLL divided (FIG. 18B) and produced IFN-γ, TNF-α and IL-2 (FIG. 18C) in a co-culture with target cells from the same subject. Proliferation and cytokine production was similar to or higher than from R12-expressing cells tested in the same condition.

The results were consistent with an observation that engineered T cells expressing the exemplary anti-ROR1 CAR F were successfully generated from primary T cells from human subjects that has CLL. The cells were viable, exhibited cytolytic activity, produced cytokines and proliferated when incubated with ROR1-expressing target cells from the same subject, in some cases even when the target cells expressed low levels of ROR1. The results supported that cells expressing the exemplary anti-ROR1 CAR F can be generated with cells from subjects with CLL, previously reported as having difficulties in generating engineered CAR-expressing cells (see, e.g., Gorgun et al., J Clin Invest. 2005 July; 115(7):1797-805; Ramsay et al., J Clin Invest. 2008 July; 118(7):2427-37; Riches et al., Discov Med. 2013 Dec.; 16(90):295-302). The results also showed that the exemplary anti-ROR1 CAR expressing cells are effective in killing target cells that express even low levels of ROR1, demonstrating a high sensitivity. By most measurements, the anti-ROR1 CAR-F-expressing cells showed improvements compared to cells expressing the anti-ROR1 reference R12 CAR.

### Example 6: Anti-Tumor Effect of Anti-ROR1 CAR-Expressing T Cells after Adoptive Transfer In Vivo in an Animal Model of a B cell malignancy

The anti-tumor effects of exemplary engineered anti-ROR1 CAR-expressing (CAR+) primary human T cells were assessed by monitoring tumors following adoptive transfer of cells in an animal model of a disseminated human B cell malignancy. The mice were administered a preparation of engineered primary human T cells generated from human donors, expressing an exemplary anti-ROR1 CAR-F, compared to a reference anti-ROR1 (R12) CAR or an anti-CD19 CAR, as described in Example 3 above.

NOD·Cg·Prkd^{scid}IL2rg^{tm1Wjl}/SzJ (NSG) mice were injected intravenously with 1.0×10⁶ firefly luciferase and green fluorescent protein (FfLuc-GFP)-expressing human mantel cell lymphoma (MCL) JeKo-1 cells on day 0. Tumor burden was assessed by bioluminescent imaging (BLI), by detecting bioluminescence minutes after mice were injected intraperitoneally with 3 mg of D-Luciferin. On day 6, mice were randomized into groups to balance tumor burden. On day 7, mice were administered engineered primary human T cells generated from a human donor expressing the exemplary anti-ROR1 CAR-F or the reference anti-ROR1 (R12) CAR, at a dose of 1×10⁶ cells (low dose) or 3×10⁶ cells (high dose) per mouse. As controls, mice either did not receive any T cells or received 3×10⁶ cells mock-treated T cells from the same donor. Mice were monitored for tumor burden and survival for 139 days post administration. The number of circulating CAR+ T cells was monitored for 28 days post administration.

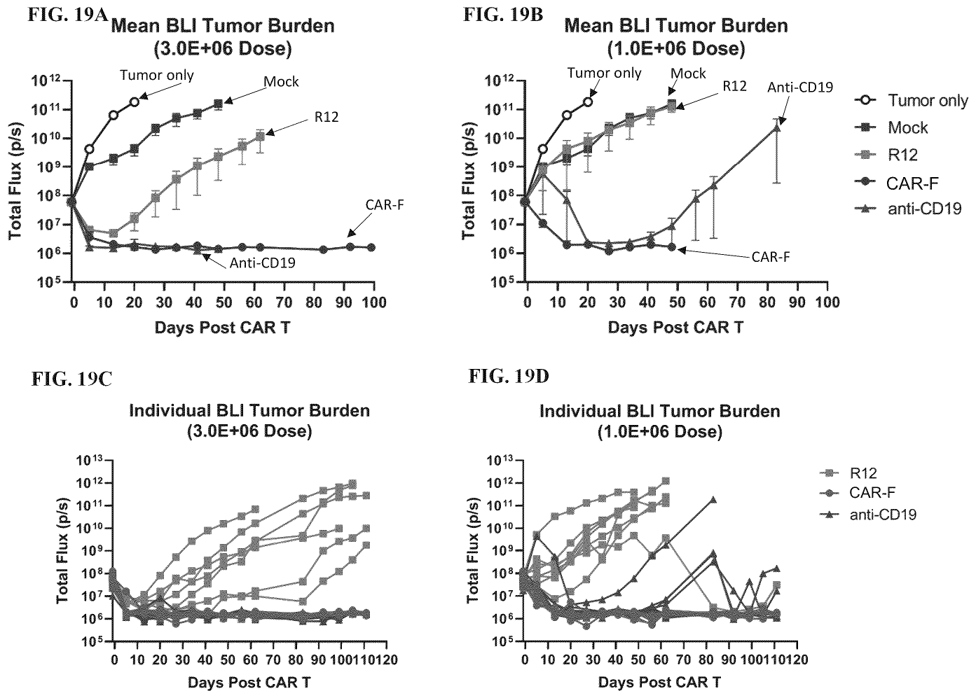

FIG. 19A (high dose) and FIG. 19B (low dose) depict mean tumor volume of all treated mice. Results from all individual mice are shown in FIG. 19C (high dose) or FIG. 19D (low dose). As shown in FIGS. 19A and 19C, JeKo-1 MCL xenograft mice administered 3×10⁶ (high dose) anti-ROR1 CAR-F expressing cells or the anti-CD19 CAR⁺ cells resulted in mean tumor regression (below the baseline) for at least 111 days (the last day of BLI measurement). In comparison, mice administered the R12 CAR⁺ cells exhibited mean tumor regression for only up to 27 days. As shown in FIGS. 19B and 19D, at the lower dose of 1×10⁶ cells, administration of cells expressing CAR-F resulted in mean tumor regression for the entire course of the study (up to 111 days). In comparison, mice administered the anti-CD19 CAR⁺ cells exhibited mean tumor regression for up to 36 days, and no mean tumor regression was observed for mice administered the R12 reference anti-ROR1 CAR.

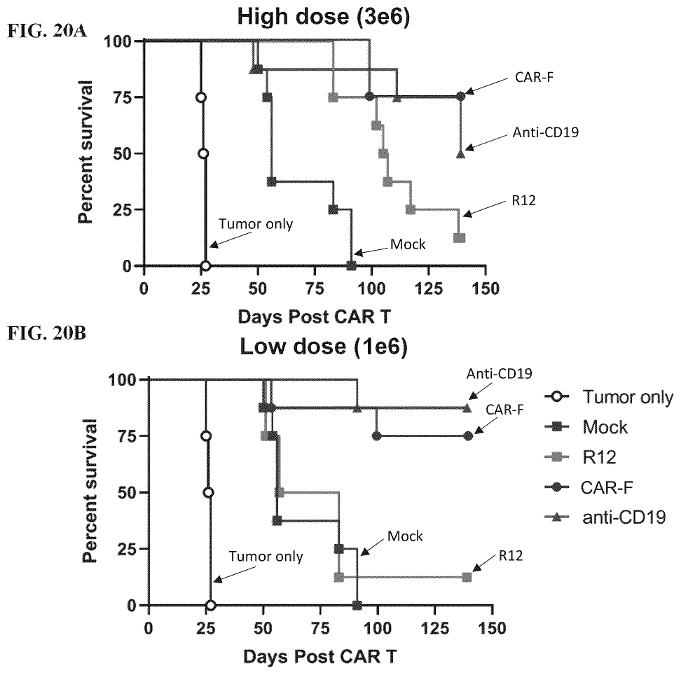

Mice were monitored for body condition at least twice weekly for 139 days post CAR T cell administration and were euthanized when moribund or exhibiting signs of severe xenogeneic graft versus host disease (xGvHD). Kaplan-Meier survival curves of each of the groups are shown in FIG. 20A (high dose) and FIG. 20B (low dose). Untreated JeKo-1 xenograft mice had a median survival of 26.5 days. Animals administered 3×10⁶ mock-treated cells had a median survival of 56 days. Median survival was unable to be determined for all animals treated with either dose of anti-ROR1 CAR-F expressing T cells, as 75% (6 of 8) of animals administered either the high dose (3×10⁶ cells; FIG. 20A) or the low dose (1×10⁶ cells; FIG. 20B) remained alive at the end of the study. Notably, the deaths observed in the mice administered the anti-ROR1 CAR-F were associated with xGvHD rather than lymphoma burden. Median survival was 70 and 106 days for animals administered R12 CAR+ T cells at the low and high doses, respectively. Median survival was unable to be determined for mice receiving anti-CD19 CAR+ T cells at the low dose (7 of 8 animals survived to study end) and the median survival was 139 days at the high dose (4 of 8 animals survived). The deaths in this group was also primarily associated with xGvHD rather than lymphoma burden.

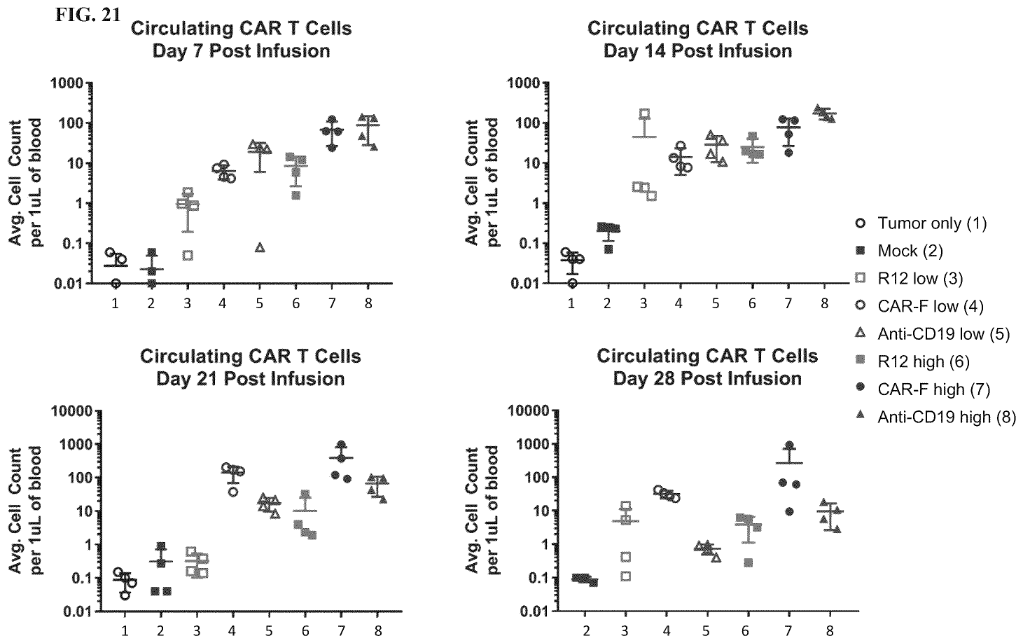

The average number of CAR+ T cells in the peripheral blood of each mouse was assessed by flow cytometry at days 7, 14, 21 and 28 after CAR+ T cell administration. As shown in FIG. 21, Anti-ROR1 CAR-F expressing cells showed the greatest concentration of circulating CAR+ T cells in the peripheral blood at day 21 or day 28 after administration at both dose levels examined. At the high dose, the anti-CD19 CAR+ T cells demonstrated substantially greater expansion (172.5 cells/pl blood) at day 14 post-administration than either the anti-ROR1 CAR-F (77.0 cells/pl blood) or R12 CAR (25.0 cells/pl blood) expressing cells. However, by day 21, anti-ROR1 CAR-F expressing cells had substantially increased in counts (140.9 cells/pl blood) compared to both R12 CAR+(0.3 cells/pl blood) and the anti-CD19 CAR+(17.2 cells/pl blood) cells at the low dose. Similar increased expansion was observed at the high dose anti-ROR1 CAR-F expressing cells, with 392.7 cells/pl blood compared to 10.2 and 66.8 cells/pl blood in the R12 CAR+ and anti-CD19 CAR⁺ cells, respectively. This improvement in expansion was also observed at day 28 post-administration of the cells.

The results supported high anti-tumor activity, improved T cell expansion, and prolonged survival of mice administered the exemplary anti-ROR1 CAR-F in the mouse xenograft model with MCL. By most measurements, the anti-ROR1 CAR-F-expressing cells showed substantial improvements compared to the anti-ROR1 reference R12 CAR, and showed similar or improved activity against MCL compared to cells expressing an anti-CD19 CAR.

### Example 7: Epitope Mapping

Epitopes recognized by the exemplary anti-ROR1 antigen-binding domain contained in the exemplary anti-ROR1 CAR-F and CAR-G (scFv sequence set forth in SEQ ID NO:118), were assessed using full discontinuous epitope mapping by Chemical Linkage of Peptides onto Scaffolds (CLIPS; Pepscan Presto BV, Lelystad, The Netherlands; see, e.g., Timmerman et al., (2007) J. Mol. Recognit. 20: 283-329). Mapping was carried out using anti-ROR1 scFv clones in which the scFv was fused with a human Fe (scFv-hFc; IgG1) or a mouse Fe (scFv-mFc).

The exemplary anti-ROR1 scFv-mFc and scFv-hFc were expressed and purified from transiently transfected HEK293 cells, and assessed by sodium dodecyl sulfate polyacrylamide gel electrophoresis (SDS-PAGE) and analytical size exclusion chromatography (SEC). The extracellular domain of ROR1 (ROR1 ECD) was recombinantly expressed and purified from transiently transfected HEK293 cells, and assessed by SDS-PAGE and SEC. The results showed a highly pure composition of anti-ROR1 scFv-hFc, scFv-mFec and ROR1-ECD (>99% pure, <1 high molecular weight multimers for scFv-hFc and ROR1-ECD; >97% pure, <3% high molecular weight multimers for scFv-mFc).

Linear and structured synthetic peptide libraries of various length were generated from human ROR1 (e.g., set forth in SEQ ID NO:144-146), each offset by one amino acid residue. The structured peptide library was prepared to fix peptides of different lengths into defined 3D structures, including any single loop, double loop, α-helical and β-turn segment of ROR1. The libraries were arranged into combinatory maps to yield combinations of loops and linear peptides representing conformational and/or discontinuous epitope of ROR1 in a non-biased manner.

An ELISA based heat map was generated on the individual and combined linear and structured peptides of ROR1 with the binders. Iterative double alanine substitution of all residues within peptides that showed positive binding were performed (for wild type alanine residues, glycine was substituted). Amino acids that resulted in a 70% or greater reduction in binding when substituted with alanine were determined to be core components of the epitope.

Analysis of epitope mapping showed that an scFv-hFC of the exemplary anti-ROR1 antigen-binding domain contained in the exemplary anti-ROR1 CAR-F and CAR-G (scFv sequence set forth in SEQ ID NO:118) bound to a discontinuous, conformation dependent epitope primarily centered around amino acids FRSTIYGSRLRIRNL (set forth in SEQ ID NO:199; corresponding to residues 78-92 of the human ROR1 sequence set forth in SEQ ID NO:144) Table E2 sets forth the epitopes identified from the epitope mapping. Most of the binding observed was to combinations with parts of FRSTIYGSRLRIRNL (SEQ ID NO:199), consistent with some cooperativity between different regions of the epitope.

TABLE E2
Binding epitopes of exemplary anti-ROR1 antigen-
binding domain

| Amino acid | | |
|---|---|---|
| residue (with | | |
| reference to | | SEQ ID |
| SEQ ID NO: 144) | Sequence | NO: |
| 5-17 | LSVSAELVPTSSW | 200 |
| 49-62 | HCKVSGNPPPTIRW | 201 |
| 62-87 | WFKNDAPVVQEPRRLSFRSTIYGSRL* | 202 |
| 111-119 | VSSTGVLFV | 203 |
| 117-129 | LFVKFGPPPTASP | 204 |
| 133-143 | DEYEEDGFCQP | 205 |
| 142-155 | QPYRGIACARFIGN* | 206 |
| 191-204 | SQFAIPSLCHYAFP | 207 |
| 202-213 | AFPYCDETSSVP | 208 |
| 228-247 | NVLCQTEYIFARSNPMILMR* | 209 |
| 248-259 | LKLPNCEDLPQP | 210 |
| 259-273 | PESPEAANCIRIGIP | 211 |
| 290-303 | VDYRGTVSVTKSGR | 212 |
| 310-323 | SQYPHTHTFTALRF | 213 |
| 366-380 | DSKEKNKMEILYILV | 214 |

Core residues, determined by ≥70% reduction in binding upon double alanine substitution, are underlined and in bold.
*likely contains multiple smaller parts of the epitope.

### Example 8: Affinity and Binding Kinetics of Anti-ROR1 Antigen-binding Domains

The binding affinity and kinetics of the exemplary anti-ROR1 antigen-binding domains to the antigen ROR1 was assessed by surface plasmon resonance (SPR).

### A. Binding Affinity and Kinetics

Equilibrium dissociation constant (K_{D}), the association rate constant (kₐ or kₒₙ), and dissociation rate constant (kₐ or k_{off}) of the interaction between recombinant human ROR1 and the binding domain of exemplary ROR1 CARs was determined by surface plasmon resonance (SPR) using the Biacore™ T200 instrument and Biacore™ T200 Evaluation software v3. Specifically, the scFv binding domain of CARs as described in Example 3 and Table E1 above, including scFv ROR1-1, ROR1-2, ROR1-3 and ROR1-4 (set forth in SEQ ID NOS: 118, 127, 109 and 134, respectively; the scFv binding domains of CAR-A, CAR-F, CAR-G, CAR-I, CAR-R and CAR-B1) and the antigen binding domain of the reference CAR R12 (SEQ ID NO: 142), were assessed.

Recombinantly generated anti-ROR1 scFv binding domains and ROR1 extracellular domain with a C-terminal 6×His tag (ROR1 ECD 6×His) were used for binding affinity and kinetics analyses. The binding domains were constructed as a fusion of the scFv with the Fc portion of murine immunoglobulin heavy chain IgG2a (scFv-mFc). The murine IgG2a domain contained 4 point mutations to diminish both Fc receptor and complement binding. Active concentration of ROR1 ECD 6×His was determined by a calibration-free concentration analysis (CFCA) assay.

For SPR, an anti-mouse IgG capture surface chip (Mouse Antibody Capture Kit type 2, catalog number 29215281 and Series S Sensor Chip CM5, catalog number 29104988, GE Healthcare Bio-Sciences) was used to capture the anti-ROR1 scFv-mFec binding domains on a flow cell while another flow cell was left blank for reference. Multi-cycle kinetics were implemented by subsequent injections of recombinant human ROR1 at concentrations of 183, 61.0, 20.33, 6.78, and 2.26 nM with variable dissociation over both the active and reference flow cells.

ROR1 ECD was injected over the scFv-mFc bound chip at multiple concentrations using multicycle kinetics. A monovalent 1:1 model was used to analyze the sensograms and to calculate affinity and kinetic measurements of the interaction.

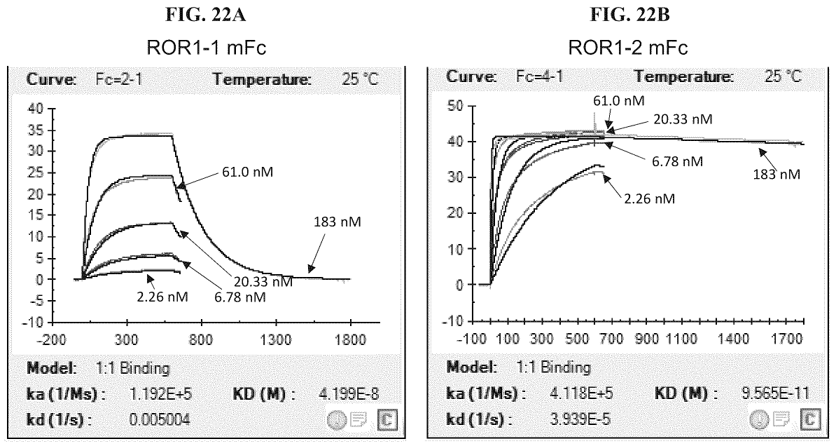

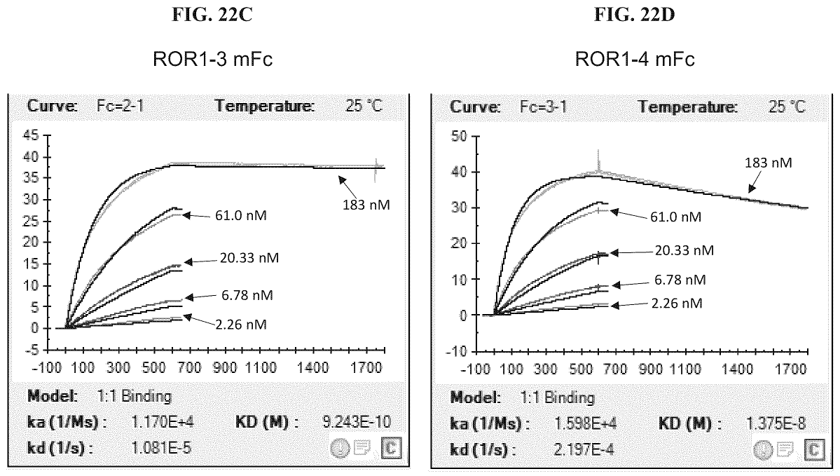

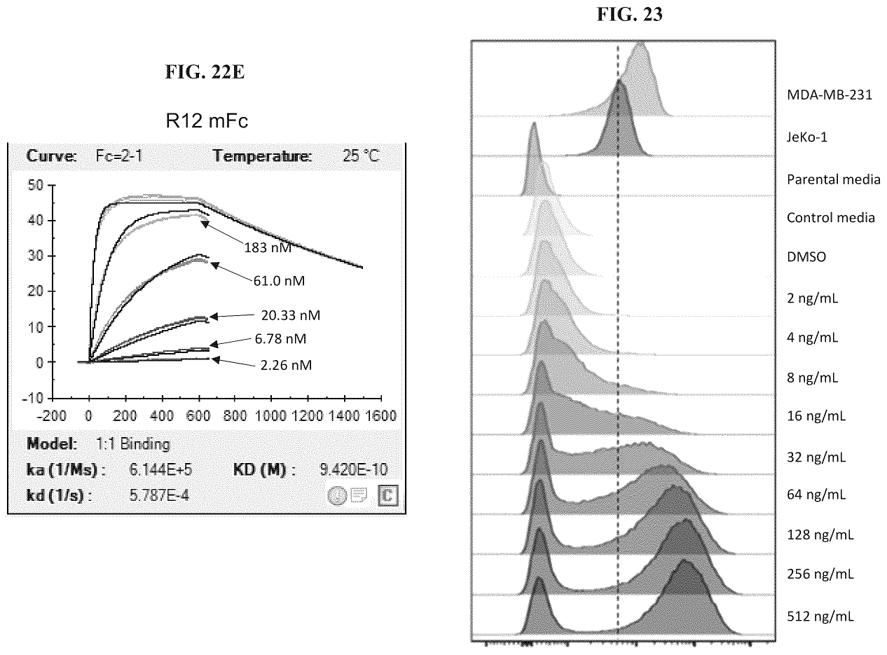

FIGS. 22A-22E depict the doubled-reference sensograms for anti-ROR1 scFv ROR1-1 (SEQ ID NO: 118; FIG. 22A), ROR1-2 (SEQ ID NO: 127; FIG. 22B), ROR1-3 (SEQ ID NO: 109; FIG. 22C) and ROR1-4 (SEQ ID NO: 134; FIG. 22D) and the scFv antigen binding domain of the reference CAR R12 (SEQ ID NO: 142; FIG. 22E). The affinity (equilibrium dissociation constant, K_{D}) of the interaction between the anti-ROR1 scFv binding domains and ROR1 are set forth in Table E3. The affinity (equilibrium dissociation constant, K_{D}) of the interaction between the anti-ROR1 scFv ROR1-1 (binding domain of CAR-F) and ROR1 was measured to be 42 nM, and the kinetic association rate constant (kₐ or kₒₙ; on-rate) and dissociation rate constant (kₐ or k_{off}; off-rate) of the interaction were observed to be kₐ=1.2×10⁵ l/Ms and k_{d}=5.0×10⁻³ 1/s, respectively. The anti-ROR1 scFv ROR1-1 (binding domain of CAR-F) exhibited substantially higher K_{D} (lower affinity) and faster off rate (kₐ or k_{off}) compared to the reference R12 scFv binding domain. The anti-ROR1 scFv ROR1-2 (binding domain of CAR-A) exhibited substantially lower K_{D} (higher affinity) and slower off rate (kₐ or k_{off}) compared to the reference R12 scFv binding domain.

TABLE E3
Affinity and binding kinetics of exemplary anti-ROR1 binding domains.

| | scFv # | | | | |
|---|---|---|---|---|---|
| | R12 | ROR1-1 | ROR1-2 | ROR1-3 | ROR1-4 |
| SEQ ID | 142 | 118 | 127 | 134 | 109 |
| NO: | | | | | |
| CAR | R12 | CAR-F, | CAR-A, | CAR-B1 | CAR-I |
| | | CAR-G | CAR-R | | |
| K_{D} (M) | 9.420E−10 | 4.20E−08 | 9.57E−11 | 1.38E−08 | 9.24E−10 |
| kₐ or kₒₙ | 6.14E+05 | 1.19E+05 | 4.12E+05 | 1.60E+04 | 1.17E+04 |
| (1/Ms) | | | | | |
| k_{d} or k_{off} | 5.79E−04 | 5.00E−03 | 3.94E−05 | 2.20E−04 | 1.08E−05 |
| (1/s) | | | | | |

### B. Epitope Binning

SPR was used in epitope binning analysis to compare the epitope binding regions recognized by the exemplary ROR1 binding domains compared to the epitope of the anti-ROR1 reference R12 binding domain. Reference R12 scFv binding domain was immobilized on the surface, ROR1 ECD 6×His was introduced and bound to the R12 binding domain, and recombinant scFv binding domains of exemplary anti-ROR1 CARs, including scFv ROR1-1, ROR1-2, ROR1-3 and ROR1-4 (set forth in SEQ ID NOS: 118, 127, 109 and 134, respectively; the scFv binding domains of CAR-A, CAR-F, CAR-G, CAR-I, CAR-R and CAR-B1) were injected to assess whether the recombinant scFv bound to similar regions of ROR1 as the R12 binding domain. The results showed that binding of the scFv R12 to the ROR1 ECD was competed for by the candidate anti-ROR1 scFv binding domains ROR1-1, ROR1-2, ROR1-3 and ROR1-4, consistent with an observation that the candidate scFv binding domains may bind to an overlapping epitope or region as that bound by the R12 binding domain.

### Example 9: Antigen Sensitivity Assessment of Anti-ROR1 Antigen-binding Domains

The antigen sensitivity of T cells expressing the exemplary anti-ROR1 CAR-F and the reference anti-ROR1 CAR R12 were evaluated for ability to produce cytokines and cytotoxic activity after stimulation using a ROR1-expressing cell line with regulatable ROR1 expression levels.

K562 human myelogenous leukemia cell line was engineered to express ROR1 in response to the tetracycline derivative, doxycycline (TetOn). As compared to the endogenously ROR1 expressing cell lines, the K562-ROR1-TetOn engineered cell line expressed very low levels of ROR1 at baseline, even in the absence of doxycycline treatment.

2×10⁴ K562-ROR1-TetOn or parental K562 target cells were transduced to stably express IncuCyte® NucLight Red. R10 culture media (RPMI 1640; 1×BME; 1×NEAA, 1×NaPyr; 10% heat inactivated FBS) were added to an optically clear 96-well flat-bottom plate. Thawed CAR-expressing T cells were re-suspended at a concentration of 1.6×10⁶ CAR⁺ cells/mL in R10 media. CAR-T were then added to K562 target cells for a final CAR+ T cell count of 8×10⁴ cells per well resulting in a 4:1 E:T ratio. Doxycycline hydrochloride was diluted to the desired concentrations (2 ng/mL-512 ng/mL) using DMSO and added to the plated CAR-T/target cell mixtures in technical triplicates. Plates were incubated at 37° C./5% CO2 for 72 hours with periodic measurement of NucLight Red fluorescence using the IncuCyte®.

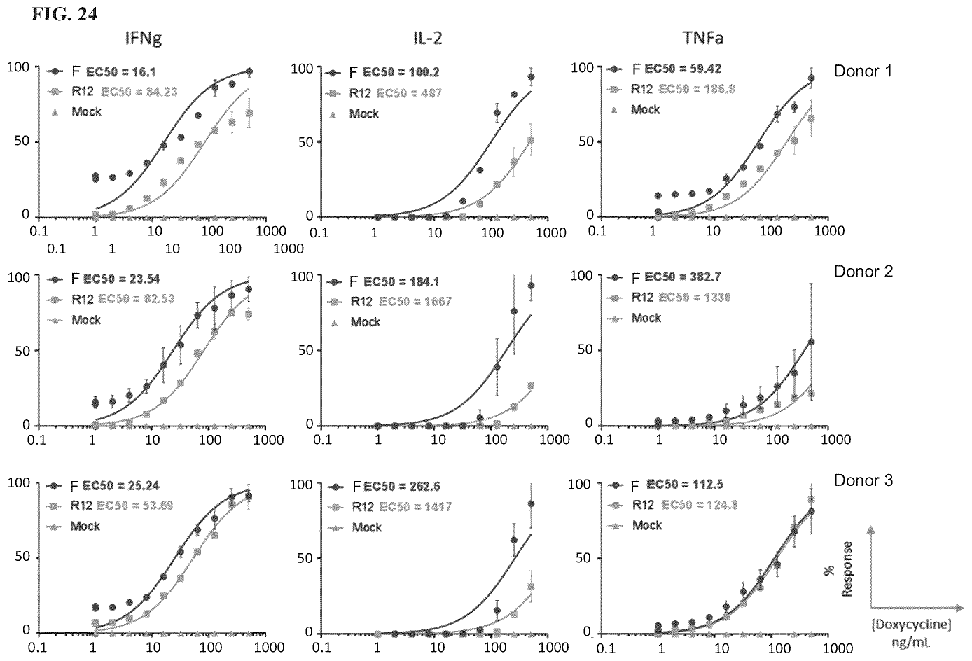

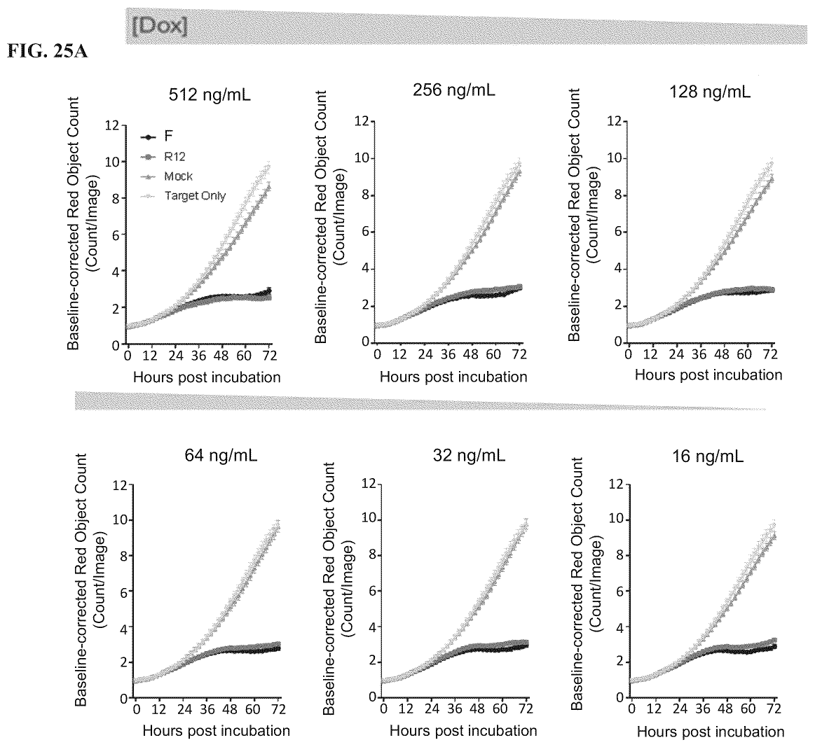

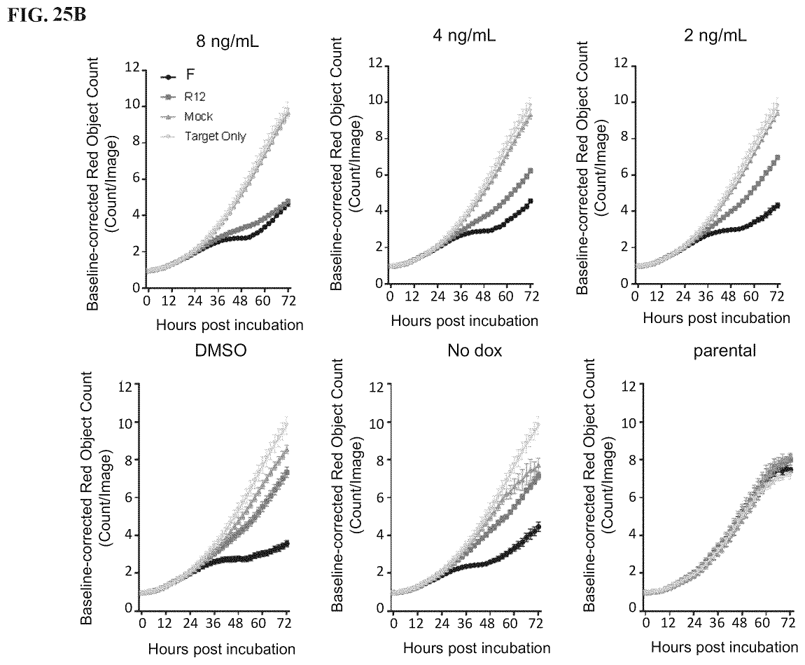

As shown in FIG. 23, ROR1 expression on K562-ROR1-TetOn cells after incubation with doxycycline alone showed that ROR1 expression increased with increasing doxycycline concentration. After 72 hours of incubation, supernatants were collected from wells containing T cells expressing anti-ROR1 CAR-F or the reference R12 CAR that had been were mixed with K562-ROR1-TetOn target cells expressing increasing amounts of ROR1. As shown in FIG. 24, CAR-F expressing T cells resulted in equivalent or greater production of IFNγ, IL-2 and TNFα compared to R12 expressing cells, at all ROR1 expression levels tested. As shown in FIGS. 25A-25B, analysis of cytotoxic function, as determined by loss of red fluorescent signal over time, showed that CAR-F expressing cells responded to lower ROR1 levels more effectively compared to R12 expressing cells, indicating greater antigenic sensitivity without the loss of specificity as demonstrated by the lack of response to the parental cell line.

### Example 10: Binding Specificity Assessment

Potential off-target binding and species cross-reactivity of the binding domain of the exemplary anti-ROR1 CAR-F was assessed by various methods.

### A. Plasma Membrane Protein Array

Off-target binding was assessed based on a plasma membrane protein array. This chip-based platform contains over 4400 groups of cells genetically engineered to overexpress individual human extracellular membrane proteins that together represent over 75% of the human extracellular proteome. The panel also includes over 1000 tethered secreted protein targets. An scFv-Fc (scFv sequence set forth in SEQ ID NO:118) was generated and used for immunohistochemistry (IHC) against the cells in the array. Confirmation screening was performed. The scFv was determined to be highly selective for ROR1, with no additional specific interactions identified, including to ROR2. These results indicate low risk for off-target activity of the anti-ROR1 CAR-F expressing cells

### B. Species Cross-Reactivity

Table E4 shows the extracellular domain (ECD) homology for ROR1 between human (Homo sapiens), Rhesus macaques (Macaca mulatta), cynomolgus macaques (Macaca fasicularis), and mice (Mus musculus). ROR1 is 100% conserved in the ECD between human and non-human primates.

TABLE E4
ROR1 ECD Homology Across Species

| | | | Mus |
|---|---|---|---|
| | Macaca mulatta | Macaca fasicularis | musculus |
| Homo sapiens | 100% | 100% | 98.7% |

Extracellular domain a.a. 30-406 of human ROR1
Homo sapiens: Uniprot Q01973 (set forth in SEQ ID NO: 214)
Macaca mulatta: Uniprot F6RUP2 (set forth in SEQ ID NO: 215)
Macaca fasicularis: Uniprot A0A2K5WTX7 (set forth in SEQ ID NO: 216), Uniprot A0A2K5WTX4 (set forth in SEQ ID NO: 217)
Mus musculus: Uniprot Q9Z139 (set forth in SEQ ID NO: 218)

Species cross-reactivity of anti-ROR1 CAR-F and R12 CAR was assessed by co-culturing an engineered Jurkat T cell line containing a Nur77-tdTomato reporter and expressing the anti-ROR1 CAR-F or the reference R12 CAR (as described above in Example 1.C), with CT26 murine fibroblasts engineered to express a mouse ROR1 (CT26-mROR1), CT26 cells engineered to express a human ROR1 (CT26-hROR1), or unmodified CT26 cells or unmodified K562 human myelogenous leukemia cells, and assessing expression of the reporter.

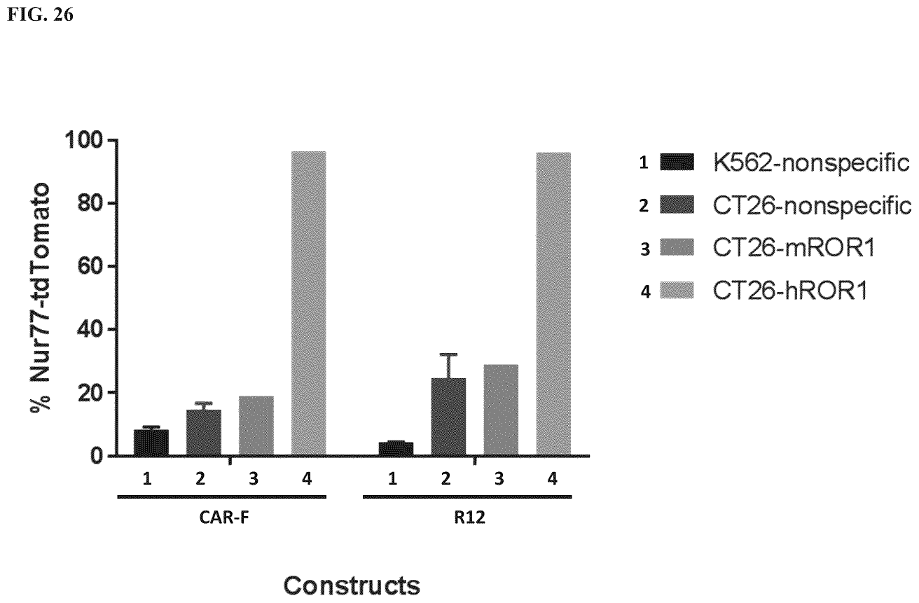

As shown in FIG. 26, although there is 98.7% homology between human and mouse ROR1, no cross-reactivity was observed for binding of the anti-ROR1 CAR-F or R12 CAR to mouse ROR1-expressing target cells.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

Sequences

| SEQ | | |
|---|---|---|
| ID | | |
| NO: | Sequence | Description |
| 1 | ESKYGPPCPPCP | Spacer (IgG4 |
| | | hinge) (aa) |
| 2 | gaatctaagtacggaccgccctgccccccttgccct | Spacer (IgG4 |
| | | hinge)(nt) |
| 3 | ESKYGPPCPPCPGQPREPQVYTLPPSQEEMTKNQVSLTCLVKGFYPSDIAVEWESNGQ | Hinge-C_{H}3 |
| | PENNYKTTPPVLDSDGSFFLYSRLTVDKSRWQEGNVFSCSVMHEALHNHYTQKSLSLS | spacer (aa) |
| | LGK | |
| 4 | GAATCTAAGTACGGACCGCCTTGTCCTCCATGTCCTGGCCAGCCAAGAGAACCCCAGG | Hinge-C_{H}3 |
| | TGTACACACTGCCTCCAAGCCAAGAGGAAATGACCAAGAACCAGGTGTCCCTGACCTG | spacer (nt) |
| | CCTGGTCAAGGGCTTCTACCCTTCCGATATCGCCGTGGAATGGGAGAGCAATGGCCAG | |
| | CCTGAGAACAACTACAAGACCACACCTCCTGTGCTGGACAGCGACGGCTCATTCTTCC | |
| | TGTACAGCCGGCTGACCGTGGACAAGAGCAGATGGCAAGAGGGCAACGTGTTCAGCTG | |
| | CAGCGTGATGCACGAGGCCCTGCACAACCACTACACCCAGAAGTCTCTGAGCCTGAGC | |
| | CTGGGCAAG | |
| 5 | RWPESPKAQASSVPTAQPQAEGSLAKATTAPATTRNTGRGGEEKKKEKEKEEQEERET | IgD-hinge-Fc |
| | KTPECPSHTQPLGVYLLTPAVQDLWLRDKATFTCFVVGSDLKDAHLTWEVAGKVPTGG | |
| | VEEGLLERHSNGSQSQHSRLTLPRSLWNAGTSVTCTLNHPSLPPQRLMALREPAAQAP | |
| | VKLSLNLLASSDPPEAASWLLCEVSGFSPPNILLMWLEDQREVNTSGFAPARPPPQPG | |
| | STTFWAWSVLRVPAPPSPQPATYTCVVSHEDSRTLLNASRSLEVSYVTDH | |
| 6 | LEGGGEGRGSLLTCGDVEENPGPR | T2A |
| 7 | MLLLVTSLLLCELPHPAFLLIPRKVCNGIGIGEFKDSLSINATNIKHFKNCTSISGDL | tEGFR |
| | HILPVAFRGDSFTHTPPLDPQELDILKTVKEITGFLLIQAWPENRTDLHAFENLEIIR | |
| | GRTKQHGQFSLAVVSLNITSLGLRSLKEISDGDVIISGNKNLCYANTINWKKLFGTSG | |
| | QKTKIISNRGENSCKATGQVCHALCSPEGCWGPEPRDCVSCRNVSRGRECVDKCNLLE | |
| | GEPREFVENSECIQCHPECLPQAMNITCTGRGPDNCIQCAHYIDGPHCVKTCPAGVMG | |
| | ENNTLVWKYADAGHVCHLCHPNCTYGCTGPGLEGCPTNGPKIPSIATGMVGALLLLLV | |
| | VALGIGLFM | |
| 8 | FWVLVVVGGVLACYSLLVTVAFIIFWV | CD28 (amino |
| | | acids 153-179 |
| | | of Accession |
| | | No. P10747) |
| 9 | IEVMYPPPYLDNEKSNGTIIHVKGKHLCPSPLFPGPSKPFWVLVVVGGVLACYSLLVT | CD28 (amino |
| | VAFIIFWV | acids 114-179 |
| | | of Accession |
| | | No. P10747) |
| 10 | RSKRSRLLHSDYMNMTPRRPGPTRKHYQPYAPPRDFAAYRS | CD28 (amino |
| | | acids 180-220 |
| | | of P10747) |
| 11 | RSKRSRGGHSDYMNMTPRRPGPTRKHYQPYAPPRDFAAYRS | CD28 (LL to |
| | | GG) |
| 12 | KRGRKKLLYIFKQPFMRPVQTTQEEDGCSCRFPEEEEGGCEL | 4-1 BB |
| | | (amino acids |
| | | 214-255 of |
| | | Q07011.1) |
| 13 | RVKFSRSADAPAYQQGQNQLYNELNLGRREEYDVLDKRRGRDPEMGGKPRRKNPQEGL | CD3 zeta |
| | YNELQKDKMAEAYSEIGMKGERRRGKGHDGLYQGLSTATKDTYDALHMQALPPR | |
| 14 | RVKFSRSAEPPAYQQGQNQLYNELNLGRREEYDVLDKRRGRDPEMGGKPRRKNPQEGL | CD3 zeta |
| | YNELQKDKMAEAYSEIGMKGERRRGKGHDGLYQGLSTATKDTYDALHMQALPPR | |
| 15 | RVKFSRSADAPAYKQGQNQLYNELNLGRREEYDVLDKRRGRDPEMGGKPRRKNPQEGL | CD3 zeta |
| | YNELQKDKMAEAYSEIGMKGERRRGKGHDGLYQGLSTATKDTYDALHMQALPPR | |
| 16 | RKVCNGIGIGEFKDSLSINATNIKHFKNCTSISGDLHILPVAFRGDSFTHTPPLDPQE | tEGFR |
| | LDILKTVKEITGFLLIQAWPENRTDLHAFENLEIIRGRTKQHGQFSLAVVSLNITSLG | |
| | LRSLKEISDGDVIISGNKNLCYANTINWKKLFGTSGQKTKIISNRGENSCKATGQVCH | |
| | ALCSPEGCWGPEPRDCVSCRNVSRGRECVDKCNLLEGEPREFVENSECIQCHPECLPQ | |
| | AMNITCTGRGPDNCIQCAHYIDGPHCVKTCPAGVMGENNTLVWKYADAGHVCHLCHPN | |
| | CTYGCTGPGLEGCPTNGPKIPSIATGMVGALLLLLVVALGIGLFM | |
| 17 | EGRGSLLTCGDVEENPGP | T2A |
| 18 | GSGATNFSLLKQAGDVEENPGP | P2A |
| 19 | ATNFSLLKQAGDVEENPGP | P2A |
| 20 | QCTNYALLKLAGDVESNPGP | E2A |
| 21 | VKQTLNFDLLKLAGDVESNPGP | F2A |
| 22 | PGGG-(SGGGG)5-P- wherein P is proline, G is glycine and S | linker |
| | is serine | |
| 23 | GSADDAKKDAAKKDGKS | Linker |
| 24 | GSTSGSGKPGSGEGSTKG | Linker |
| 25 | X₁PPX₂P X1 is glycine, cysteine or arginine X2 is cysteine | Hinge |
| | or threonine | |
| 26 | EPKSCDKTHTCPPCP | Hinge |
| 27 | ERKCCVECPPCP | Hinge |
| 28 | ELKTPLGDTHTCPRCPEPKSCDTPPPCPRCPEPKSCDTPPPCPRCPEPKSCDTPPPCP | Hinge |
| | RCP | |
| 29 | ESKYGPPCPSCP | Hinge |
| 30 | gagtctaaatacggaccgccttgtcctccttgtccc | Spacer |
| | | (IgG4hinge) |
| | | (nt) O/SSE |
| 31 | YGPPCPPCP | Hinge |
| 32 | KYGPPCPPCP | Hinge |
| 33 | EVVVKYGPPCPPCP | Hinge |
| 34 | ESKYGPPCPPCPAPPVAGPSVFLFPPKPKDTLMISRTPEVTCVVVDVSQEDPEVQFNW | Hinge-C_{H}2- |
| | YVDGVEVHNAKTKPREEQFQSTYRVVSVLTVLHQDWLNGKEYKCKVSNKGLPSSIEKT | C_{H}3 spacer |
| | ISKAKGQPREPQVYTLPPSQEEMTKNQVSLTCLVKGFYPSDIAVEWESNGQPENNYKT | aa |
| | TPPVLDSDGSFFLYSRLTVDKSRWQEGNVFSCSVMHEALHNHYTQKSLSLSLGK | |
| 35 | GAATCTAAGTACGGACCGCCTTGTCCTCCATGTCCTGCTCCTCCAGTTGCCGGACCTT | Hinge-C_{H}2- |
| | CCGTGTTCCTGTTTCCTCCAAAGCCTAAGGACACCCTGATGATCAGCAGAACCCCTGA | C_{H}3 spacer |
| | AGTGACCTGCGTGGTGGTGGACGTGTCCCAAGAGGATCCTGAGGTGCAGTTCAACTGG | (nt |
| | TATGTGGACGGCGTGGAAGTGCACAACGCCAAGACCAAGCCTAGAGAGGAACAGTTCC | |
| | AGAGCACCTACAGAGTGGTGTCCGTGCTGACAGTGCTGCACCAGGATTGGCTGAACGG | |
| | CAAAGAGTACAAGTGCAAGGTGTCCAACAAGGGCCTGCCTAGCAGCATCGAGAAAACC | |
| | ATCAGCAAGGCCAAGGGCCAGCCAAGAGAACCCCAGGTGTACACACTGCCTCCAAGCC | |
| | AAGAGGAAATGACCAAGAACCAGGTGTCCCTGACCTGCCTGGTCAAGGGCTTCTACCC | |
| | TTCCGATATCGCCGTGGAATGGGAGAGCAATGGCCAGCCTGAGAACAACTACAAGACC | |
| | ACACCTCCTGTGCTGGACAGCGACGGCTCATTCTTCCTGTACAGCCGGCTGACCGTGG | |
| | ACAAGAGCAGATGGCAAGAGGGCAACGTGTTCAGCTGCAGCGTGATGCACGAGGCCCT | |
| | GCACAACCACTACACCCAGAAGTCTCTGAGCCTGAGCCTGGGCAAG | |
| 36 | ESKYGPPCPPCPAPEFLGGPSVFLFPPKPKDTLMISRTPEVTCVVVDVSQEDPEVQFN | Hinge-C_{H}2- |
| | WYVDGVEVHNAKTKPREEQFNSTYRVVSVLTVLHQDWLNGKEYKCKVSNKGLPSSIEK | C_{H}3 spacer |
| | TISKAKGQPREPQVYTLPPSQEEMTKNQVSLTCLVKGFYPSDIAVEWESNGQPENNYK | (aa) |
| | TTPPVLDSDGSFFLYSRLTVDKSRWQEGNVFSCSVMHEALHNHYTQKSLSLSLGK | |
| 37 | ESKYGPPCPPCPAPPVAGPSVFLFPPKPKDTLMISRTPEVTCVVVDVSQEDPEVQFNW | IgG4/IgG2 |
| | YV | hinge- |
| | DGVEVHNAKTKPREEQFQSTYRVVSVLTVLHQDWLNGKEYKCKVSNKGLPSSIEKTIS | IgG2/IgG4 |
| | KA | C_{H}2-IgG4 |
| | KGQPREPQVYTLPPSQEEMTKNQVSLTCLVKGFYPSDIAVEWESNGQPENNYKTTPPV | C_{H}3 spacer |
| | LD SDGSFFLYSRLTVDKSRWQEGNVFSCSVMHEALHNHYTQKSLSLSLGK | (aa) |
| 38 | gaatctaagtacggaccgccctgccctccctgccctgctcctcctgtggctggaccaa | IgG4/IgG2 |
| | gcgtgttcctgtttccacctaagcctaaagataccctgatgatttcccgcacacctga | hinge- |
| | agtgacttgcgtggtcgtggacgtgagccaggaggatccagaagtgcagttcaactgg | IgG2/IgG4 |
| | tacgtggacggcgtggaagtccacaatgctaagactaaaccccgagaggaacagtttc | C_{H}2- IgG4 |
| | agtcaacttaccgggtcgtgagcgtgctgaccgtcctgcatcaggattggctgaacgg | C_{H}3 spacer |
| | gaaggagtataagtgcaaagtgtctaataagggactgcctagctccatcgagaaaaca | (nt) |
| | attagtaaggcaaaagggcagcctcgagaaccacaggtgtataccctgccccctagcc | |
| | aggaggaaatgaccaagaaccaggtgtccctgacatgtctggtcaaaggcttctatcc | |
| | aagtgacatcgccgtggagtgggaatcaaatgggcagcccgagaacaattacaagacc | |
| | acaccacccgtgctggactctgatggaagtttctttctgtattccaggctgaccgtgg | |
| | ataaatctcgctggcaggagggcaacgtgttctcttgcagtgtcatgcacgaagccct | |
| | gcacaatcattatacacagaagtcactgagcctgtccctgggcaaa | |
| 39 | GGGGS | 4GS linker |
| | | (aa) |
| 40 | GGGS | 3GS linker |
| | | (aa) |
| 41 | GGGGSGGGGSGGGGS | (4GS)₃ linker |
| | | (aa) |
| 42 | MPLLLLLPLLWAGALA | CD33 Signal |
| | | Peptide |
| 43 | MVLQTQVFISLLLWISGAYG | human IgG- |
| | | kappa signal |
| | | peptide (aa) |
| 44 | atggtgctgcagacccaggtgttcatcagcctgctgctgtggatctccggagcatacg | human IgG- |
| | ga | kappa signal |
| | | sequence (nt) |
| 45 | MLLLVTSLLLCELPHPAFLLIP | GMCSFR |
| | | alpha chain |
| | | signal peptide |
| | | (aa) |
| 46 | atgcttctcctggtgacaagccttctgctctgtgagttaccacacccagcattcctcc | GMCSFR |
| | tgatccca | alpha chain |
| | | signal |
| | | sequence (nt) |
| 47 | MALPVTALLLPLALLLHA | CD8 alpha |
| | | signal peptide |
| 48 | ASTKGPSVFPLAPCSRSTSESTAALGCLVKDYFPEPVTVSWNSGALTSGVHTFPAVLQ | Human IgG2 |
| | SSGLYSLSSVVTVPSSNFGTQTYTCNVDHKPSNTKVDKTVERKCCVECPPCPAPPVAG | Fc (Uniprot |
| | PSVFLFPPKPKDTLMISRTPEVTCVVVDVSHEDPEVQFNWYVDGVEVHNAKTKPREEQ | P01859) |
| | FNSTFRVVSVLTVVHQDWLNGKEYKCKVSNKGLPAPIEKTISKTKGQPREPQVYTLPP | |
| | SREEMTKNQVSLTCLVKGFYPSDISVEWESNGQPENNYKTTPPMLDSDGSFFLYSKLT | |
| | VDKSRWQQGNVFSCSVMHEALHNHYTQKSLSLSPGK | |
| 49 | ASTKGPSVFPLAPCSRSTSESTAALGCLVKDYFPEPVTVSWNSGALTSGVHTFPAVLQ | Human IgG4 |
| | SSGLYSLSSVVTVPSSSLGTKTYTCNVDHKPSNTKVDKRVESKYGPPCPSCPAPEFLG | Fc (Uniprot |
| | GPSVFLFPPKPKDTLMISRTPEVTCVVVDVSQEDPEVQFNWYVDGVEVHNAKTKPREE | P01861) |
| | QFNSTYRVVSVLTVLHQDWLNGKEYKCKVSNKGLPSSIEKTISKAKGQPREPQVYTLP | |
| | PSQEEMTKNQVSLTCLVKGFYPSDIAVEWESNGQPENNYKTTPPVLDSDGSFFLYSRL | |
| | TVDKSRWQEGNVFSCSVMHEALHNHYTQKSLSLSLGK | |
| 50 | GYTFTSY | CDR-H1 |
| 51 | GYTFTSYGIS | CDR-H1 |
| 52 | SYGIS | CDR-H1 |
| 53 | GYTFTSYG | CDR-H1 |
| 54 | SAYNGN | CDR-H2 |
| 55 | WISAYNGNTK | CDR-H2 |
| 56 | WISAYNGNTKYAQKLQG | CDR-H2 |
| 57 | ISAYNGNT | CDR-H2 |
| 58 | DEDILTGYNYYGMDV | CDR-H3 |
| 59 | ARDEDILTGYNYYGMDV | CDR-H3 |
| 60 | TLSSGHSSYAILA | CDR-L1 |
| 61 | SGHSSYA, | CDR-L1 |
| 62 | LNSDGSHSKGD | CDR-L2 |
| 63 | LNSDGSH | CDR-L2 |
| 64 | QTWGTGIRV | CDR-L3 |
| 65 | GGSISNY | CDR-H1 |
| 66 | GGSISNYYWS | CDR-H1 |
| 67 | NYYWS | CDR-H1 |
| 68 | GGSISNYY | CDR-H1 |
| 69 | YTSGS | CDR-H2 |
| 70 | RIYTSGSTN | CDR-H2 |
| 71 | RIYTSGSTNYNPSLKS | CDR-H2 |
| 72 | IYTSGST | CDR-H2 |
| 73 | YYDILTGFFDY | CDR-H3 |
| 74 | ARYYDILTGFFDY | CDR-H3 |
| 75 | RMSQDISSYLA | CDR-L1 |
| 76 | QDISSY | CDR-L1 |
| 77 | AASSLQS | CDR-L2 |
| 78 | AAS | CDR-L2 |
| 79 | QQYDSFPPT | CDR-L3 |
| 80 | GGSINSTTS | CDR-H1 |
| 81 | GGSINSTTSYWA | CDR-H1 |
| 82 | STTSYWA | CDR-H1 |
| 83 | GGSINSTTSY | CDR-H1 |
| 84 | FYSGK | CDR-H2 |
| 85 | TIFYSGKTY | CDR-H2 |
| 86 | TIFYSGKTYNNPSLKS | CDR-H2 |
| 87 | IFYSGKT | CDR-H2 |
| 88 | FDYGFHDAFDI | CDR-H3 |
| 89 | ARFDYGFHDAFDI | CDR-H3 |
| 90 | RASQSITSDYLS | CDR-L1 |
| 91 | QSITSDY | CDR-L1 |
| 92 | GASTRAT | CDR-L2 |
| 93 | GAS | CDR-L2 |
| 94 | QQDYNLTYT | CDR-L3 |
| 95 | SAYTGN | CDR-H2 |
| 96 | WISAYTGNTR | CDR-H2 |
| 97 | WISAYTGNTRYAQKLOG | CDR-H2 |
| 98 | ISAYTGNT | CDR-H2 |
| 99 | EEGATTDYDYYGMDV | CDR-H3 |
| 100 | AREEGATTDYDYYGMDV | CDR-H3 |
| 101 | CAGGTTCAGCTGGTGCAGTCTGGAGCTGAGGTGAAGAAGCCTGGGGCCTCAGTGAAGG | V_{H} (nt) |
| | TCTCCTGCAAGGCTTCTGGTTACACCTTTACCAGCTATGGTATCAGCTGGGTGCGACA | |
| | GGCCCCTGGACAAGGGCTTGAGTGGATGGGATGGATCAGCGCTTACAATGGTAACACA | |
| | AAGTATGCACAGAAGCTCCAGGGCAGAGTCACCATGACCACAGACACATCCACGAGCA | |
| | CAGCCTACATGGAGCTGAGGAGCCTGAGATCTGACGACACGGCCGTGTATTACTGTGC | |
| | GAGAGATGAGGATATTTTGACTGGTTACAACTACTACGGTATGGACGTCTGGGGCCAA | |
| | GGGACCACGGTCACCGTCTCCTCA | |
| 102 | caggtgcagctggttcaatctggcgccgaagtgaagaaaccaggcgcctctgtgaagg | V_{H} (nt) |
| | tgtcctgcaaggccagcggctacacctttaccagctacggcatcagctgggtccgaca | O/SSE |
| | ggctcctggacaaggcttggaatggatgggctggatcagcgcctacaacggcaacacc | |
| | aaatacgcccagaaactgcagggcagagtgaccatgaccaccgacaccagcacaagca | |
| | ccgcctacatggaactgcggagcctgagatccgatgacaccgccgtgtactactgcgc | |
| | cagagatgaggacatcctgaccggctacaactactacggcatggacgtgtggggccag | |
| | ggcacaacagtgacagtttcttct | |
| 103 | QVQLVQSGAEVKKPGASVKVSCKASGYTFTSYGISWVRQAPGQGLEWMGWISAYNGNT | V_{H} (aa) |
| | KYAQKLQGRVTMTTDTSTSTAYMELRSLRSDDTAVYYCARDEDILTGYNYYGMDVWGQ | |
| | GTTVTVSS | |
| 104 | CAGCTTGTGCTGACTCAATCGCCCTCTGCCTCTGCCTCCCTGGGAGCCTCGGTCAAGC | V_{L} (nt) |
| | TCACCTGCACTCTGAGCAGTGGGCACAGCAGCTACGCCATCGCATGGCATCAGCAGCA | |
| | GCCAGAGAAGGGCCCTCGGTACTTGATGAAGCTTAACAGTGATGGCAGCCACAGCAAG | |
| | GGGGACGGGATCCCTGATCGCTTCTCAGGCTCCAGCTCTGGGGCTGAGCGCTACCTCA | |
| | CCATCTCCAGCCTCCAGTCTGAGGATGAGGCTGACTATTACTGTCAGACCTGGGGCAC | |
| | TGGCATTCGGGTGTTCGGTGGAGGAACCAAACTGACTGTCCTAGGC | |
| 105 | caactggtgctgacacagtctcctagcgcctctgcttctctgggagccagcgtgaagc | V_{L} (nt) |
| | tgacctgtacactgtctagcggccacagcagctacgccattgcttggcatcagcagca | O/SSE |
| | gcccgagaagggccctagatacctgatgaagctgaacagcgacggcagccactctaaa | |
| | ggcgacggcatccccgatagattcagcggcagttctagcggagccgagcgctacctga | |
| | caatcagctctctgcaatccgaggacgaggccgactactactgtcagacatggggcac | |
| | cggcatcagagtgtttggcggaggcaccaagctgacagtgcttgga | |
| 106 | QLVLTQSPSASASLGASVKLTCTLSSGHSSYAIAWHQQQPEKGPRYLMKLNSDGSHSK | V_{L} (aa) |
| | GDGIPDRFSGSSSGAERYLTISSLQSEDEADYYCQTWGTGIRVFGGGTKLTVLG | |
| 107 | CAGGTTCAGCTGGTGCAGTCTGGAGCTGAGGTGAAGAAGCCTGGGGCCTCAGTGAAGG | scFv (V_{H}-V_{L}) |
| | TCTCCTGCAAGGCTTCTGGTTACACCTTTACCAGCTATGGTATCAGCTGGGTGCGACA | (nt) |
| | GGCCCCTGGACAAGGGCTTGAGTGGATGGGATGGATCAGCGCTTACAATGGTAACACA | |
| | AAGTATGCACAGAAGCTCCAGGGCAGAGTCACCATGACCACAGACACATCCACGAGCA | |
| | CAGCCTACATGGAGCTGAGGAGCCTGAGATCTGACGACACGGCCGTGTATTACTGTGC | |
| | GAGAGATGAGGATATTTTGACTGGTTACAACTACTACGGTATGGACGTCTGGGGCCAA | |
| | GGGACCACGGTCACCGTCTCCTCAGGTGGTGGTGGTAGCGGCGGCGGCGGCTCTGGTG | |
| | GTGGTGGATCCCAGCTTGTGCTGACTCAATCGCCCTCTGCCTCTGCCTCCCTGGGAGC | |
| | CTCGGTCAAGCTCACCTGCACTCTGAGCAGTGGGCACAGCAGCTACGCCATCGCATGG | |
| | CATCAGCAGCAGCCAGAGAAGGGCCCTCGGTACTTGATGAAGCTTAACAGTGATGGCA | |
| | GCCACAGCAAGGGGGACGGGATCCCTGATCGCTTCTCAGGCTCCAGCTCTGGGGCTGA | |
| | GCGCTACCTCACCATCTCCAGCCTCCAGTCTGAGGATGAGGCTGACTATTACTGTCAG | |
| | ACCTGGGGCACTGGCATTCGGGTGTTCGGTGGAGGAACCAAACTGACTGTCCTAGGC | |
| 108 | caggtgcagctggttcaatctggcgccgaagtgaagaaaccaggcgcctctgtgaagg | scFv (V_{H}-V_{L}) |
| | tgtcctgcaaggccagcggctacacctttaccagctacggcatcagctgggtccgaca | (nt) O/SSE |
| | ggctcctggacaaggcttggaatggatgggctggatcagcgcctacaacggcaacacc | |
| | aaatacgcccagaaactgcagggcagagtgaccatgaccaccgacaccagcacaagca | |
| | ccgcctacatggaactgcggagcctgagatccgatgacaccgccgtgtactactgcgc | |
| | cagagatgaggacatcctgaccggctacaactactacggcatggacgtgtggggccag | |
| | ggcacaacagtgacagtttcttctggcggcggaggatctggcggaggtggaagcggag | |
| | gcggtggatctcaactggtgctgacacagtctcctagcgcctctgcttctctgggagc | |
| | cagcgtgaagctgacctgtacactgtctagcggccacagcagctacgccattgcttgg | |
| | catcagcagcagcccgagaagggccctagatacctgatgaagctgaacagcgacggca | |
| | gccactctaaaggcgacggcatccccgatagattcagcggcagttctagcggagccga | |
| | gcgctacctgacaatcagctctctgcaatccgaggacgaggccgactactactgtcag | |
| | acatggggcaccggcatcagagtgtttggcggaggcaccaagctgacagtgcttgga | |
| 109 | QVQLVQSGAEVKKPGASVKVSCKASGYTFTSYGISWVRQAPGQGLEWMGWISAYNGNT | scFv (V_{H}-V_{L}) |
| | KYAQKLQGRVTMTTDTSTSTAYMELRSLRSDDTAVYYCARDEDILTGYNYYGMDVWGQ | (aa) |
| | GTTVTVSSGGGGSGGGGSGGGGSQLVLTQSPSASASLGASVKLTCTLSSGHSSYAIAW | |
| | HQQQPEKGPRYLMKLNSDGSHSKGDGIPDRFSGSSSGAERYLTISSLQSEDEADYYCQ | |
| | TWGTGIRVFGGGTKLTVLG | |
| 110 | CAGGTGCAGCTGCAGGAGTCGGGCCCAGGACTGGTGAAGCCTTCGGAGACCCTGTCCC | V_{H} (nt) |
| | TCACCTGCACTGTCTCTGGAGGCTCCATCAGTAATTACTACTGGAGCTGGATCCGGCA | |
| | GCCCGCCGGGAAGGGACTGGAGTGGATTGGGCGTATCTATACCAGTGGGAGCACCAAC | |
| | TACAACCCCTCCCTCAAGAGTCGAGTCACCATGTCAGTAGACACGTCCAAGAACCAGT | |
| | TCTCCCTGAAGCTGAGTTCTTTGACCGCCGCGGACACGGCCATATATTACTGTGCGAG | |
| | GTATTACGATATTTTGACTGGTTTCTTTGACTACTGGGGCCAGGGAACCCTGGTCACC | |
| | GTCTCCTCA | |
| 111 | Caggttcagctgcaagagtctggccctggcctggtcaagcctagcgaaacactgagcc | V_{H} (nt) |
| | tgacctgtaccgtgtctggcggcagcatctccaactactactggtcctggatcagaca | O/SSE |
| | gcctgccggcaaaggcctggaatggatcggcagaatctacaccagcggcagcaccaac | |
| | tacaaccccagcctgaagtccagagtgaccatgagcgtggacaccagcaagaaccagt | |
| | tctccctgaagctgagcagcctgacagccgccgataccgccatctactactgtgcccg | |
| | gtactacgatatcctgaccggcttcttcgactactggggccagggaacactggtcaca | |
| | gtttctagc | |
| 112 | QVQLQESGPGLVKPSETLSLTCTVSGGSISNYYWSWIRQPAGKGLEWIGRIYTSGSTN | V_{H} (aa) |
| | YNPSLKSRVTMSVDTSKNQFSLKLSSLTAADTAIYYCARYYDILTGFFDYWGQGTLVT | |
| | VSS | |
| 113 | GTCATCTGGATGACCCAGTCTCCATCCTTACTCTCTGCATCTACAGGAGACAGTGTCA | V_{L} (nt) |
| | CCATCAGTTGTCGGATGAGTCAGGACATTAGCAGTTATTTAGCCTGGTATCAGCAAAA | |
| | ACCAGGGAAAGCCCCTGAGCTCCTGATCTATGCTGCATCCAGTTTGCAAAGTGGGGTC | |
| | CCATCAAGGTTCAGTGGCAGTGGATCTGGGACAGACTTCACTCTCACCATCAGTTCCC | |
| | TGCAGTCTGAAGATTTTGCTACTTATTACTGTCAACAGTATGATAGTTTCCCTCCGAC | |
| | GTTCGGCCAAGGGACCAAGGTGGAATTCAAACGG | |
| 114 | Gtgatttggatgacacagagccctagcctgctgagcgccagcacaggcgatagcgtga | VL (nt) |
| | ccatcagctgcagaatgagccaggacatcagcagctacctggcttggtatcagcagaa | O/SSE |
| | gcctggcaaggcccctgaactgctgatctatgccgcttccagtctgcagagcggcgtg | |
| | ccatctagattttccggcagcggctctggcaccgacttcaccctgacaatcagctccc | |
| | tgcagtccgaggacttcgccacctactattgccagcagtacgacagcttccctccaac | |
| | ctttggccagggcaccaaggtggaattcaagcgc | |
| 115 | VIWMTQSPSLLSASTGDSVTISCRMSQDISSYLAWYQQKPGKAPELLIYAASSLQSGV | V_{L} (aa) |
| | PSRFSGSGSGTDFTLTISSLQSEDFATYYCQQYDSFPPTFGQGTKVEFKR | |
| 116 | CAGGTGCAGCTGCAGGAGTCGGGCCCAGGACTGGTGAAGCCTTCGGAGACCCTGTCCC | scFv (V_{H}-V_{L}) |
| | TCACCTGCACTGTCTCTGGAGGCTCCATCAGTAATTACTACTGGAGCTGGATCCGGCA | (nt) |
| | GCCCGCCGGGAAGGGACTGGAGTGGATTGGGCGTATCTATACCAGTGGGAGCACCAAC | |
| | TACAACCCCTCCCTCAAGAGTCGAGTCACCATGTCAGTAGACACGTCCAAGAACCAGT | |
| | TCTCCCTGAAGCTGAGTTCTTTGACCGCCGCGGACACGGCCATATATTACTGTGCGAG | |
| | GTATTACGATATTTTGACTGGTTTCTTTGACTACTGGGGCCAGGGAACCCTGGTCACC | |
| | GTCTCCTCAGGTGGTGGTGGTAGCGGCGGCGGCGGCTCTGGTGGTGGTGGATCCGTCA | |
| | TCTGGATGACCCAGTCTCCATCCTTACTCTCTGCATCTACAGGAGACAGTGTCACCAT | |
| | CAGTTGTCGGATGAGTCAGGACATTAGCAGTTATTTAGCCTGGTATCAGCAAAAACCA | |
| | GGGAAAGCCCCTGAGCTCCTGATCTATGCTGCATCCAGTTTGCAAAGTGGGGTCCCAT | |
| | CAAGGTTCAGTGGCAGTGGATCTGGGACAGACTTCACTCTCACCATCAGTTCCCTGCA | |
| | GTCTGAAGATTTTGCTACTTATTACTGTCAACAGTATGATAGTTTCCCTCCGACGTTC | |
| | GGCCAAGGGACCAAGGTGGAATTCAAACGG | |
| 117 | caggttcagctgcaagagtctggccctggcctggtcaagcctagcgaaacactgagcc | scFV (V_{H}-V_{L}) |
| | tgacctgtaccgtgtctggcggcagcatctccaactactactggtcctggatcagaca | (nt) O/SSE |
| | gcctgccggcaaaggcctggaatggatcggcagaatctacaccagcggcagcaccaac | |
| | tacaaccccagcctgaagtccagagtgaccatgagcgtggacaccagcaagaaccagt | |
| | tctccctgaagctgagcagcctgacagccgccgataccgccatctactactgtgcccg | |
| | gtactacgatatcctgaccggcttcttcgactactggggccagggaacactggtcaca | |
| | gtttctagcggaggcggaggatctggtggcggaggaagtggcggaggcggttctgtga | |
| | tttggatgacacagagccctagcctgctgagcgccagcacaggcgatagcgtgaccat | |
| | cagctgcagaatgagccaggacatcagcagctacctggcttggtatcagcagaagcct | |
| | ggcaaggcccctgaactgctgatctatgccgcttccagtctgcagagcggcgtgccat | |
| | ctagattttccggcagcggctctggcaccgacttcaccctgacaatcagctccctgca | |
| | gtccgaggacttcgccacctactattgccagcagtacgacagcttccctccaaccttt | |
| | ggccagggcaccaaggtggaattcaagcgc | |
| 118 | QVQLQESGPGLVKPSETLSLTCTVSGGSISNYYWSWIRQPAGKGLEWIGRIYTSGSTN | sCFV (V_{H}-V_{L}) |
| | YNPSLKSRVTMSVDTSKNQFSLKLSSLTAADTAIYYCARYYDILTGFFDYWGQGTLVT | (aa) |
| | VSSGGGGSGGGGSGGGGSVIWMTQSPSLLSASTGDSVTISCRMSQDISSYLAWYQQKP | |
| | GKAPELLIYAASSLQSGVPSRFSGSGSGTDFTLTISSLQSEDFATYYCQQYDSFPPTF | |
| | GQGTKVEFKR | |
| 119 | CAGCTGCAGCTGCAGGAGTCGGGCCCAGGACTGGTGAAGCCTTCGGAGACCCTGTCCC | V_{H} (nt) |
| | TCACCTGCACTGTCTCTGGTGGCTCCATCAACAGTACTACTTCCTACTGGGCCTGGAT | |
| | CCGCCAGCCCCCAGGGAAGGGGCTGGAGTGGATTGGGACTATCTTTTATAGTGGGAAA | |
| | ACCTACAACAACCCGTCCCTCAAGAGTCGAGTCACCATGTCCGTAGACACGTCCAAGA | |
| | ACCACTTCTCCCTGAAGGTGAACTCTGTGACCGCCGCAGACACGGCTGTGTATTACTG | |
| | TGCGAGGTTTGACTACGGTTTTCATGATGCTTTTGATATCTGGGGCCAGGGGACAATG | |
| | GTCACCGTCTCTTCA | |
| 120 | cagctccagctgcaagaatctggacctggcctggtcaagcccagcgagacactgtctc | V_{H} (nt) |
| | tgacctgtacagtgtccggcggcagcatcaatagcaccacaagctactgggcctggat | O/SSE |
| | cagacagcctcctggcaaaggcctggaatggatcggcaccatcttctacagcggcaag | |
| | acctacaacaaccccagcctgaagtccagagtgaccatgagcgtggacaccagcaaga | |
| | accacttcagcctgaaagtgaacagcgtgacagccgccgataccgccgtgtactactg | |
| | cgccagattcgactacggcttccacgacgccttcgacatctggggccagggcacaatg | |
| | gtcacagtttctagc | |
| 121 | QLQLQESGPGLVKPSETLSLTCTVSGGSINSTTSYWAWIRQPPGKGLEWIGTIFYSGK | V_{H} (aa) |
| | TYNNPSLKSRVTMSVDTSKNHFSLKVNSVTAADTAVYYCARFDYGFHDAFDIWGQGTM | |
| | VTVSS | |
| 122 | GAAATTGTAATGACACAGTCTCCAGCCACCCTGTCTTTGTCTCCAGGGGAAAGAGCCA | V_{L} (nt) |
| | CCCTCTCCTGCAGGGCCAGTCAGAGTATTACCAGCGACTACTTATCCTGGTACCAACA | |
| | AAAACCTGGGCAGGCTCCCAGGCTCCTCATCTATGGTGCATCCACCAGGGCCACTGGC | |
| | ATCCCAGCCAGGTTCAGTGGCAGTGGGTCTGGGACAGACTTCACTCTCACCATCAGCA | |
| | GCCTGCAGCCTGAAGATTTTGTAGTTTATTACTGTCAGCAGGATTATAACTTGTACAC | |
| | TTTTGGCCAGGGGACCAAGCTGGAGATCAAACGG | |
| 123 | gagattgtgatgacacagagccccgccactctgagccttagtcctggcgaaagagcca | V_{L} (nt) |
| | cactgagctgcagagccagccagagcatcaccagcgattacctgagctggtatcagca | O/SSE |
| | gaagcccggacaggctcccagactgctgatctatggcgcctctacaagagccaccggc | |
| | attcccgcccgcttttctggctctggaagcggcaccgacttcaccctgaccatatcta | |
| | gcctgcagcctgaggacttcgtggtgtactattgccagcaggactacaacctgtacac | |
| | cttcggccaggggaccaagctggaaatcaagaga | |
| 124 | EIVMTQSPATLSLSPGERATLSCRASQSITSDYLSWYQQKPGQAPRLLIYGASTRATG | V_{L} (aa) |
| | IPARFSGSGSGTDFTLTISSLQPEDFVVYYCQQDYNLYTFGQGTKLEIKR | |
| 125 | CAGCTGCAGCTGCAGGAGTCGGGCCCAGGACTGGTGAAGCCTTCGGAGACCCTGTCCC | scFv (V_{H}-V_{L}) |
| | TCACCTGCACTGTCTCTGGTGGCTCCATCAACAGTACTACTTCCTACTGGGCCTGGAT | (nt) |
| | CCGCCAGCCCCCAGGGAAGGGGCTGGAGTGGATTGGGACTATCTTTTATAGTGGGAAA | |
| | ACCTACAACAACCCGTCCCTCAAGAGTCGAGTCACCATGTCCGTAGACACGTCCAAGA | |
| | ACCACTTCTCCCTGAAGGTGAACTCTGTGACCGCCGCAGACACGGCTGTGTATTACTG | |
| | TGCGAGGTTTGACTACGGTTTTCATGATGCTTTTGATATCTGGGGCCAGGGGACAATG | |
| | GTCACCGTCTCTTCAGGTGGTGGTGGTAGCGGCGGCGGCGGCTCTGGTGGTGGTGGAT | |
| | CCGAAATTGTAATGACACAGTCTCCAGCCACCCTGTCTTTGTCTCCAGGGGAAAGAGC | |
| | CACCCTCTCCTGCAGGGCCAGTCAGAGTATTACCAGCGACTACTTATCCTGGTACCAA | |
| | CAAAAACCTGGGCAGGCTCCCAGGCTCCTCATCTATGGTGCATCCACCAGGGCCACTG | |
| | GCATCCCAGCCAGGTTCAGTGGCAGTGGGTCTGGGACAGACTTCACTCTCACCATCAG | |
| | CAGCCTGCAGCCTGAAGATTTTGTAGTTTATTACTGTCAGCAGGATTATAACTTGTAC | |
| | ACTTTTGGCCAGGGGACCAAGCTGGAGATCAAACGG | |
| 126 | Cagctccagctgcaagaatctggacctggcctggtcaagcccagcgagacactgtctc | scFv (V_{H}-V_{L}) |
| | tgacctgtacagtgtccggcggcagcatcaatagcaccacaagctactgggcctggat | (nt) O/SSE |
| | cagacagcctcctggcaaaggcctggaatggatcggcaccatcttctacagcggcaag | |
| | acctacaacaaccccagcctgaagtccagagtgaccatgagcgtggacaccagcaaga | |
| | accacttcagcctgaaagtgaacagcgtgacagccgccgataccgccgtgtactactg | |
| | cgccagattcgactacggcttccacgacgccttcgacatctggggccagggcacaatg | |
| | gtcacagtttctagcggaggcggaggatctggtggcggaggaagtggcggaggcggtt | |
| | ctgagattgtgatgacacagagccccgccactctgagccttagtcctggcgaaagagc | |
| | cacactgagctgcagagccagccagagcatcaccagcgattacctgagctggtatcag | |
| | cagaagcccggacaggctcccagactgctgatctatggcgcctctacaagagccaccg | |
| | gcattcccgcccgcttttctggctctggaagcggcaccgacttcaccctgaccatatc | |
| | tagcctgcagcctgaggacttcgtggtgtactattgccagcaggactacaacctgtac | |
| | accttcggccaggggaccaagctggaaatcaagaga | |
| 127 | QLQLQESGPGLVKPSETLSLTCTVSGGSINSTTSYWAWIRQPPGKGLEWIGTIFYSGK | scFv (V_{H}-V_{L}) |
| | TYNNPSLKSRVTMSVDTSKNHFSLKVNSVTAADTAVYYCARFDYGFHDAFDIWGQGTM | (aa) |
| | VTVSSGGGGSGGGGSGGGGSEIVMTQSPATLSLSPGERATLSCRASQSITSDYLSWYQ | |
| | QKPGQAPRLLIYGASTRATGIPARFSGSGSGTDFTLTISSLQPEDFVVYYCQQDYNLY | |
| | TFGQGTKLEIKR | |
| 128 | CAGGTTCAGCTGCTGCAGTCTGGAGCTGAGGTGAAGAAGCCTGGGGCCTCAGTGAAGG | V_{H} (nt) |
| | TCTCCTGCAAGGCTTCTGGTTACACCTTTACCAGCTATGGTATCAGCTGGGTGCGACA | |
| | GGCCCCTGGACAAGGGCTTGAGTGGATGGGATGGATCAGCGCTTACACTGGTAACACA | |
| | AGGTATGCACAGAAGCTCCAGGGCAGAGTCACCATGACCACAGACACATCCACGAGCA | |
| | CAGCCTACATGGAGCTGAGGAGCCTGAGATCTGACGACACGGCCGTGTATTACTGTGC | |
| | GAGAGAAGAAGGAGCTACTACGGACTACGACTACTACGGTATGGACGTCTGGGGCCAA | |
| | GGGACTGCGGTCACCGTCTCCTCA | |
| 129 | caggttcagctgcttcagtctggcgccgaagtgaagaaacctggcgcctctgtgaagg | V_{H} (nt) |
| | tgtcctgcaaggccagcggctacacctttaccagctacggcatcagctgggtccgaca | O/SSE |
| | ggctcctggacaaggcttggaatggatgggctggatcagcgcctacaccggcaatacc | |
| | agatacgcccagaaactgcagggcagagtgaccatgaccaccgacaccagcacaagca | |
| | ccgcctacatggaactgcggagcctgagatccgatgacaccgccgtgtactactgcgc | |
| | cagagaagaaggcgccaccaccgactacgactactacggcatggatgtgtggggccag | |
| | ggaacagccgtgacagtttcttct | |
| 130 | QVQLLQSGAEVKKPGASVKVSCKASGYTFTSYGISWVRQAPGQGLEWMGWISAYTGNT | V_{H} (aa) |
| | RYAQKLQGRVTMTTDTSTSTAYMELRSLRSDDTAVYYCAREEGATTDYDYYGMDVWGQ | |
| | GTAVTVSS | |
| 131 | caactggttctgacacagagcccaagcgcctctgcatctctgggagcttccgtgaagc | V_{L} (nt) |
| | tgacctgcacactgtctagcggccacagcagctatgccattgcctggcatcagcaaca | O/SSE |
| | gcccgagaagggccctagatacctgatgaagctgaacagcgacggcagccactctaaa | |
| | ggcgacggcatccccgatagattcagcggcagttctagcggagccgagcgctacctga | |
| | caatcagctctctgcaatccgaggacgaggccgattactactgtcagacatggggcac | |
| | cggcatcagagtgtttggcggcggaacaaagctgaccgtgctgggc | |
| 132 | CAGGTTCAGCTGCTGCAGTCTGGAGCTGAGGTGAAGAAGCCTGGGGCCTCAGTGAAGG | scFv (V_{H}-V_{L}) |
| | TCTCCTGCAAGGCTTCTGGTTACACCTTTACCAGCTATGGTATCAGCTGGGTGCGACA | (nt) |
| | GGCCCCTGGACAAGGGCTTGAGTGGATGGGATGGATCAGCGCTTACACTGGTAACACA | |
| | AGGTATGCACAGAAGCTCCAGGGCAGAGTCACCATGACCACAGACACATCCACGAGCA | |
| | CAGCCTACATGGAGCTGAGGAGCCTGAGATCTGACGACACGGCCGTGTATTACTGTGC | |
| | GAGAGAAGAAGGAGCTACTACGGACTACGACTACTACGGTATGGACGTCTGGGGCCAA | |
| | GGGACTGCGGTCACCGTCTCCTCAGGTGGTGGTGGTAGCGGCGGCGGCGGCTCTGGTG | |
| | GTGGTGGATCCCAGCTTGTGCTGACTCAATCGCCCTCTGCCTCTGCCTCCCTGGGAGC | |
| | CTCGGTCAAGCTCACCTGCACTCTGAGCAGTGGGCACAGCAGCTACGCCATCGCATGG | |
| | CATCAGCAGCAGCCAGAGAAGGGCCCTCGGTACTTGATGAAGCTTAACAGTGATGGCA | |
| | GCCACAGCAAGGGGGACGGGATCCCTGATCGCTTCTCAGGCTCCAGCTCTGGGGCTGA | |
| | GCGCTACCTCACCATCTCCAGCCTCCAGTCTGAGGATGAGGCTGACTATTACTGTCAG | |
| | ACCTGGGGCACTGGCATTCGGGTGTTCGGTGGAGGAACCAAACTGACTGTCCTAGGC | |
| 133 | caggttcagctgcttcagtctggcgccgaagtgaagaaacctggcgcctctgtgaagg | scFv (V_{H}-V_{L}) |
| | tgtcctgcaaggccagcggctacacctttaccagctacggcatcagctgggtccgaca | (nt) O/SSE |
| | ggctcctggacaaggcttggaatggatgggctggatcagcgcctacaccggcaatacc | |
| | agatacgcccagaaactgcagggcagagtgaccatgaccaccgacaccagcacaagca | |
| | ccgcctacatggaactgcggagcctgagatccgatgacaccgccgtgtactactgcgc | |
| | cagagaagaaggcgccaccaccgactacgactactacggcatggatgtgtggggccag | |
| | ggaacagccgtgacagtttcttctggtggcggaggatctggcggaggtggaagcggcg | |
| | gaggcggatctcaactggttctgacacagagcccaagcgcctctgcatctctgggagc | |
| | ttccgtgaagctgacctgcacactgtctagcggccacagcagctatgccattgcctgg | |
| | catcagcaacagcccgagaagggccctagatacctgatgaagctgaacagcgacggca | |
| | gccactctaaaggcgacggcatccccgatagattcagcggcagttctagcggagccga | |
| | gcgctacctgacaatcagctctctgcaatccgaggacgaggccgattactactgtcag | |
| | acatggggcaccggcatcagagtgtttggcggcggaacaaagctgaccgtgctgggc | |
| 134 | QVQLLQSGAEVKKPGASVKVSCKASGYTFTSYGISWVRQAPGQGLEWMGWISAYTGNT | scFv (V_{H}-V_{L}) |
| | RYAQKLQGRVTMTTDTSTSTAYMELRSLRSDDTAVYYCAREEGATTDYDYYGMDVWGQ | (aa) |
| | GTAVTVSSGGGGSGGGGSGGGGSQLVLTQSPSASASLGASVKLTCTLSSGHSSYAIAW | |
| | HQQQPEKGPRYLMKLNSDGSHSKGDGIPDRFSGSSSGAERYLTISSLQSEDEADYYCQ | |
| | TWGTGIRVFGGGTKLTVLG | |
| 135 | ESKYGPPCPPCPM | IgG4 hinge |
| | | spacer (aa) |
| 136 | gagtctaaatacggaccgccttgtcctccttgtcccatg | IgG4 hinge |
| | | spacer (nt) |
| | | O/SSE |
| 137 | gagtctaaatacggaccgccttgtcctccttgtcccggccagccaagagagccccagg | Hinge-C_{H}3 |
| | tttacacactgcctccaagccaagaggaaatgaccaagaatcaggtgtccctgacatg | spacer (nt) |
| | cctggtcaagggcttctacccctccgatatcgccgtggaatgggagagcaatggccag | O/SSE |
| | cctgagaacaactacaagaccacacctcctgtgctggacagcgacggcagtttcttcc | |
| | tgtatagtagactcaccgtggataaatcaagatggcaagagggcaacgtgttcagctg | |
| | cagcgtgatgcacgaggccctgcacaaccactacacccagaaaagcctgagcctgtct | |
| | ctgggcaag | |
| 138 | ESKYGPPCPPCPGQPREPQVYTLPPSQEEMTKNQVSLTCLVKGFYPSDIAVEWESNGQ | Hinge-C_{H}3 |
| | PENNYKTTPPVLDSDGSFFLYSRLTVDKSRWQEGNVFSCSVMHEALHNHYTQKSLSLS | spacer (aa) |
| | LGKM | |
| 139 | gagtctaaatacggaccgccttgtcctccttgtcccggccagccaagagagccccagg | Hinge-C_{H}3 |
| | tttacacactgcctccaagccaagaggaaatgaccaagaatcaggtgtccctgacatg | spacer (nt) |
| | cctggtcaagggcttctacccctccgatatcgccgtggaatgggagagcaatggccag | O/SSE |
| | cctgagaacaactacaagaccacacctcctgtgctggacagcgacggcagtttcttcc | |
| | tgtatagtagactcaccgtggataaatcaagatggcaagagggcaacgtgttcagctg | |
| | cagcgtgatgcacgaggccctgcacaaccactacacccagaaaagcctgagcctgtct | |
| | ctgggcaagatg | |
| 140 | gagtctaaatacggaccgccttgtcctccttgtcccgctcctcctgttgccggacctt | IgG4/IgG2 |
| | ccgtgttcctgtttcctccaaagcctaaggacaccctgatgatcagcaggacccctga | hinge- |
| | agtgacctgcgtggtggtggatgtgtcccaagaggatcccgaggtgcagttcaactgg | IgG2/IgG4 |
| | tatgtggacggcgtggaagtgcacaacgccaagaccaagcctagagaggaacagttcc | C_{H}2-IgG4 |
| | agagcacctacagagtggtgtccgtgctgacagtgctgcaccaggattggctgaacgg | C_{H}3 spacer |
| | caaagagtacaagtgcaaggtgtccaacaagggcctgcctagcagcatcgagaaaacc | (nt) O/SSE |
| | atctccaaggccaagggccagccaagagagccccaggtttacacactgcctccaagcc | |
| | aagaggaaatgaccaagaatcaggtgtccctgacatgcctggtcaagggcttctaccc | |
| | ctccgatatcgccgtggaatgggagagcaatggccagcctgagaacaactacaagacc | |
| | acacctcctgtgctggacagcgacggcagtttcttcctgtatagtagactcaccgtgg | |
| | ataaatcaagatggcaagagggcaacgtgttcagctgcagcgtgatgcacgaggccct | |
| | gcacaaccactacacccagaaaagcctgagcctgtctctgggcaag | |
| 141 | cttgaaggtggtggcgaaggcagaggcagcctgcttacatgcggagatgtggaagaga | T2A (nt) |
| | accccggacctaga | O/SSE |
| 142 | QEQLVESGGRLVTPGGSLTLSCKASGFDFSAYYMSWVRQAPGKGLEWIATIYPSSGKT | R12 V_{H}-V_{L} |
| | YYATWVNGRFTISSDNAQNTVDLQMNSLTAADRATYFCARDSYADDGALFNIWGPGTL | scFv (aa) |
| | VTISSGGGGSGGGGSGGGGSELVLTQSPSVSAALGSPAKITCTLSSAHKTDTIDWYQQ | |
| | LQGEAPRYLMQVQSDGSYTKRPGVPDRFSGSSSGADRYLIIPSVQADDEADYYCGADY | |
| | IGGYVFGGGTQLTVTG | |
| 143 | caagaacagctggtggaatctggcggcagactggttacacctggcggaagcctgacac | R12 V_{H}-V_{L} |
| | tgagctgtaaagccagcggcttcgacttcagcgcctactacatgagctgggtccgaca | scFv (nt) |
| | ggcccctggcaaaggactggaatggatcgccacaatctaccccagctccggcaagacc | |
| | tactacgccacatgggtcaacggccggttcaccatcagcagcgacaacgcccagaaca | |
| | ccgtggacctgcagatgaactctctgacagccgccgaccgggccacctacttttgtgc | |
| | cagagatagctacgccgacgacggcgccctgttcaatatttggggacctggcacactc | |
| | gtgaccatctctagcggaggcggaggaagtggtggcggaggatcaggcggtggtggat | |
| | ctgaactggtgctgacacagagcccctctgtgtctgctgctctgggaagccctgccaa | |
| | gatcacatgtaccctgagcagcgcccacaagaccgacaccatcgactggtatcagcag | |
| | ctgcagggcgaagcccctagatacctgatgcaggttcagagcgacggcagctacacca | |
| | aaagacctggcgtgcccgatagattcagcggcagttcttctggcgccgatcgctacct | |
| | gatcatcccttctgtgcaagccgacgatgaggccgactattactgcggagccgattac | |
| | atcggcggctacgttttcggtggcggcacacagttgacagtgacaggcg | |
| 144 | QETELSVSAELVPTSSWNISSELNKDSYLTLDEPMNNITTSLGQTAELHCKVSGNPPP | human ROR1 |
| | TIRWFKNDAPVVQEPRRLSFRSTIYGSRLRIRNLDTTDTGYFQCVATNGKEVVSSTGV | (aa) |
| | LFVKFGPPPTASPGYSDEYEEDGFCQPYRGIACARFIGNRTVYMESLHMQGEIENQIT | GenBank: |
| | AAFTMIGTSSHLSDKCSQFAIPSLCHYAFPYCDETSSVPKPRDLCRDECEILENVLCQ | AAA60275.1 |
| | TEYIFARSNPMILMRLKLPNCEDLPQPESPEAANCIRIGIPMADPINKNHKCYNSTGV | |
| | DYRGTVSVTKSGRQCQPWNSQYPHTHTFTALRFPELNGGHSYCRNPGNQKEAPWCFTL | |
| | DENFKSDLCDIPACDSKDSKEKNKMEILYILVPSVAIPLAIALLFFFICVCRNNQKSS | |
| | SAPVQRQPKHVRGQNVEMSMLNAYKPKSKAKELPLSAVRFMEELGECAFGKIYKGHLY | |
| | LPGMDHAQLVAIKTLKDYNNPQQWTEFQQEASLMAELHHPNIVCLLGAVTQEQPVCML | |
| | FEYINQGDLHEFLIMRSPHSDVGCSSDEDGTVKSSLDHGDFLHIAIQIAAGMEYLSSH | |
| | FFVHKDLAARNILIGEQLHVKISDLGLSREIYSADYYRVQSKSLLPIRWMPPEAIMYG | |
| | KFSSDSDIWSFGVVLWEIFSFGLQPYYGFSNQEVIEMVRKRQLLPCSEDCPPRMYSLM | |
| | TECWNEIPSRRPRFKDIHVRLRSWEGLSSHTSSTTPSGGNATTQTTSLSASPVSNLSN | |
| | PRYPNYMFPSQGITPQGQIAGFIGPPIPQNQRFIPINGYPIPPGYAAFPAAHYQPTGP | |
| | PRVIQHCPPPKSRSPSSASGSTSTGHVTSLPSSGSNQEANIPLLPHMSIPNHPGGMGI | |
| | TVFGNKSQKPYKIDSKQASLLGDANIHGHTESMISAEL | |
| 145 | QETELSVSAELVPTSSWNISSELNKDSYLTLDEPMNNITTSLGQTAELHCKVSGNPPP | human ROR1 |
| | TIRWFKNDAPVVQEPRRLSFRSTIYGSRLRIRNLDTTDTGYFQCVATNGKEVVSSTGV | isoform 2 (aa) |
| | LFVKFGPPPTASPGYSDEYEEDGFCQPYRGIACARFIGNRTVYMESLHMQGEIENQIT | |
| | AAFTMIGTSSHLSDKCSQFAIPSLCHYAFPYCDETSSVPKPRDLCRDECEILENVLCQ | |
| | TEYIFARSNPMILMRLKLPNCEDLPQPESPEAANCIRIGIPMADPINKNHKCYNSTGV | |
| | DYRGTVSVTKSGRQCQPWNSQYPHTHTFTALRFPELNGGHSYCRNPGNQKEAPWCFTL | |
| | DENFKSDLCDIPACDSKDSKEKNKMEILYILVPSVAIPLAIALLFFFICVCRNNQKSS | |
| | SAPVQRQPKHVRGQNVEMSMLNAYKPKSKAKELPLSAVRFMEELGECAFGKIYKGHLY | |
| | LPGMDHAQLVAIKTLKDYNNPQQWTEFQQEASLMAELHHPNIVCLLGAVTQEQPVC | |
| 146 | QETELSVSAELVPTSSWNISSELNKDSYLTLDEPMNNITTSLGQTAELHCKVSGNPPP | human ROR1 |
| | TIRWFKNDAPVVQEPRRLSFRSTIYGSRLRIRNLDTTDTGYFQCVATNGKEVVSSTGV | isoform 3 (aa) |
| | LFVKFGPPPTASPGYSDEYEEDGFCQPYRGIACARFIGNRTVYMESLHMQGEIENQIT | |
| | AAFTMIGTSSHLSDKCSQFAIPSLCHYAFPYCDETSSVPKPRDLCRDECEILENVLCQ | |
| | TEYIFARSNPMILMRLKLPNCEDLPQPESPEAANCIRIGIPMADPINKNHKCYNSTGV | |
| | DYRGTVSVTKSGRQCQPWNSQYPHTHTFTALRFPELNGGHSYCRNPGNQKEAPWCFTL | |
| | DENFKSDLCDIPACGK | |
| 147 | atgttttgggtgctggtcgtggtcggaggggtgctggcctgttacagcctgctggtga | CD28 |
| | cagtcgctttcatcatcttctgggtg | transmembrane |
| | | domain (nt) |
| 148 | atgttctgggtgctcgtggtcgttggcggagtgctggcctgttacagcctgctggtta | CD28 |
| | ccgtggccttcatcatcttttgggtc | transmembrane |
| | | domain (nt) |
| | | (O/SSE) |
| 149 | MFWVLVVVGGVLACYSLLVTVAFIIFWV | CD28 |
| | | transmembrane |
| | | domain (aa) |
| 150 | agagtgaagttcagcagatccgccgacgctccagcctatcagcagggccaaaaccagc | CD3-zeta |
| | tgtacaacgagctgaacctggggagaagagaagagtacgacgtgctggataagcggag | derived |
| | aggcagagatcctgaaatgggcggcaagcccagacggaagaatcctcaagagggcctg | intracellular |
| | tataatgagctgcagaaagacaagatggccgaggcctacagcgagatcggaatgaagg | signaling |
| | gcgagcgcagaagaggcaagggacacgatggactgtaccagggcctgagcaccgccac | domain (nt) |
| | caaggatacctatgacgcactgcacatgcaggccctgccacctaga | (O/SSE) |
| 151 | atgcttctcctggtgacaagccttctgctctgtgagttaccacacccagcattcctcc | truncated |
| | tgatcccacgcaaagtgtgtaacggaataggtattggtgaatttaaagactcactctc | EGFR |
| | cataaatgctacgaatattaaacacttcaaaaactgcacctccatcagtggcgatctc | (tEGFR) |
| | cacatcctgccggtggcatttaggggtgactccttcacacatactcctcctctggatc | sequence (nt) |
| | cacaggaactggatattctgaaaaccgtaaaggaaatcacagggtttttgctgattca | |
| | ggcttggcctgaaaacaggacggacctccatgcctttgagaacctagaaatcatacgc | |
| | ggcaggaccaagcaacatggtcagttttctcttgcagtcgtcagcctgaacataacat | |
| | ccttgggattacgctccctcaaggagataagtgatggagatgtgataatttcaggaaa | |
| | caaaaatttgtgctatgcaaatacaataaactggaaaaaactgtttgggacctccggt | |
| | cagaaaaccaaaattataagcaacagaggtgaaaacagctgcaaggccacaggccagg | |
| | tctgccatgccttgtgctcccccgagggctgctggggcccggagcccagggactgcgt | |
| | ctcttgccggaatgtcagccgaggcagggaatgcgtggacaagtgcaaccttctggag | |
| | ggtgagccaagggagtttgtggagaactctgagtgcatacagtgccacccagagtgcc | |
| | tgcctcaggccatgaacatcacctgcacaggacggggaccagacaactgtatccagtg | |
| | tgcccactacattgacggcccccactgcgtcaagacctgcccggcaggagtcatggga | |
| | gaaaacaacaccctggtctggaagtacgcagacgccggccatgtgtgccacctgtgcc | |
| | atccaaactgcacctacggatgcactgggccaggtcttgaaggctgtccaacgaatgg | |
| | gcctaagatcccgtccatcgccactgggatggtgggggccctcctcttgctgctggtg | |
| | gtggccctggggatcggcctcttcatgtga | |
| 152 | atgctgctcctcgtgacaagcctgctcctgtgtgaactccctcatccagcttttctgc | truncated |
| | tcattcctcggaaagtgtgcaacggcatcggcatcggagagttcaaggacagcctgag | EGFR |
| | catcaatgccaccaacatcaagcacttcaagaattgcaccagcatcagcggcgacctg | (tEGFR) |
| | cacattctgcctgtggcctttagaggcgacagcttcacccacacacctccactggatc | sequence (nt) |
| | cccaagagctggatatcctgaaaaccgtgaaagagattaccggattcctcctgatcca | (O/SSE) |
| | agcctggccagagaacagaaccgatctgcacgccttcgagaacctcgagatcatcaga | |
| | ggccggaccaaacagcacggccagtttagcctggctgtggtgtctctgaacatcacca | |
| | gtctgggcctgagaagcctgaaagaaatctccgacggcgacgtgatcatctccggaaa | |
| | caagaacctgtgctacgccaacaccatcaactggaagaagctgttcggcacctccggc | |
| | cagaaaacaaagatcatctctaaccggggcgagaacagctgcaaggccaccggacaag | |
| | tttgtcacgccctgtgtagccctgaaggctgttggggacccgaacctagagactgtgt | |
| | gtcctgccggaatgtgtcccggggcagagaatgtgtggataagtgcaacctgctggaa | |
| | ggcgagccccgcgagtttgtggaaaacagcgagtgcatccagtgtcaccccgagtgtc | |
| | tgccccaggccatgaacattacatgcaccggcagaggccccgacaactgtattcagtg | |
| | cgcccactacatcgacggccctcactgcgtgaaaacatgtccagctggcgtgatggga | |
| | gagaacaacaccctcgtgtggaagtatgccgacgccggacatgtgtgccacctgtgtc | |
| | accctaattgcacctacggctgtaccggacctggcctggaaggatgccctacaaacgg | |
| | ccctaagatccccagcattgccaccggaatggttggagccctgctgcttctgttggtg | |
| | gtggccctcggaatcggcctgttcatgtga | |
| 153 | MLLLVTSLLLCELPHPAFLLIPRKVCNGIGIGEFKDSLSINATNIKHFKNCTSISGDL | truncated |
| | HILPVAFRGDSFTHTPPLDPQELDILKTVKEITGFLLIQAWPENRTDLHAFENLEIIR | EGFR |
| | GRTKQHGQFSLAVVSLNITSLGLRSLKEISDGDVIISGNKNLCYANTINWKKLFGTSG | (tEGFR) |
| | QKTKIISNRGENSCKATGQVCHALCSPEGCWGPEPRDCVSCRNVSRGRECVDKCNLLE | sequence (aa) |
| | GEPREFVENSECIQCHPECLPQAMNITCTGRGPDNCIQCAHYIDGPHCVKTCPAGVMG | |
| | ENNTLVWKYADAGHVCHLCHPNCTYGCTGPGLEGCPTNGPKIPSIATGMVGALLLLLV | |
| | VALGIGLFM | |
| 154 | aagcgggggagaaagaaactgctgtatattttcaaacagccctttatgagacctgtgc | 4-1BB |
| | agactacccaggaggaagacggatgcagctgtaggtttcccgaggaagaggaaggagg | intracellular |
| | ctgtgagctg | co-signaling |
| | | sequence (nt) |
| 155 | aagcggggcagaaagaagctgctctacatcttcaagcagcccttcatgcggcccgtgc | 4-1BB |
| | agaccacacaagaggaagatggctgctcctgcagattccccgaggaagaagaaggcgg | intracellular |
| | ctgcgagctg | co-signaling |
| | | sequence (nt)- |
| | | (O/SSE) |
| 156 | caggttcagctgcaagagtctggccctggcctggtcaagcctagcgaaacactgagcc | anti-ROR1 |
| | tgacctgtaccgtgtctggcggcagcatctccaactactactggtcctggatcagaca | CAR (nt) |
| | gcctgccggcaaaggcctggaatggatcggcagaatctacaccagcggcagcaccaac | |
| | tacaaccccagcctgaagtccagagtgaccatgagcgtggacaccagcaagaaccagt | |
| | tctccctgaagctgagcagcctgacagccgccgataccgccatctactactgtgcccg | |
| | gtactacgatatcctgaccggcttcttcgactactggggccagggaacactggtcaca | |
| | gtttctagcggaggcggaggatctggtggcggaggaagtggcggaggcggttctgtga | |
| | tttggatgacacagagccctagcctgctgagcgccagcacaggcgatagcgtgaccat | |
| | cagctgcagaatgagccaggacatcagcagctacctggcttggtatcagcagaagcct | |
| | ggcaaggcccctgaactgctgatctatgccgcttccagtctgcagagcggcgtgccat | |
| | ctagattttccggcagcggctctggcaccgacttcaccctgacaatcagctccctgca | |
| | gtccgaggacttcgccacctactattgccagcagtacgacagcttccctccaaccttt | |
| | ggccagggcaccaaggtggaattcaagcgcgagtctaaatacggaccgccttgtcctc | |
| | cttgtcccatgttctgggtgctcgtggtcgttggcggagtgctggcctgttacagcct | |
| | gctggttaccgtggccttcatcatcttttgggtcaagcggggcagaaagaagctgctc | |
| | tacatcttcaagcagcccttcatgcggcccgtgcagaccacacaagaggaagatggct | |
| | gctcctgcagattccccgaggaagaagaaggcggctgcgagctgagagtgaagttcag | |
| | cagatccgccgacgctccagcctatcagcagggccaaaaccagctgtacaacgagctg | |
| | aacctggggagaagagaagagtacgacgtgctggataagcggagaggcagagatcctg | |
| | aaatgggcggcaagcccagacggaagaatcctcaagagggcctgtataatgagctgca | |
| | gaaagacaagatggccgaggcctacagcgagatcggaatgaagggcgagcgcagaaga | |
| | ggcaagggacacgatggactgtaccagggcctgagcaccgccaccaaggatacctatg | |
| | acgcactgcacatgcaggccctgccacctaga | |
| 157 | cagctccagctgcaagaatctggacctggcctggtcaagcccagcgagacactgtctc | anti-ROR1 |
| | tgacctgtacagtgtccggcggcagcatcaatagcaccacaagctactgggcctggat | CAR (nt) |
| | cagacagcctcctggcaaaggcctggaatggatcggcaccatcttctacagcggcaag | |
| | acctacaacaaccccagcctgaagtccagagtgaccatgagcgtggacaccagcaaga | |
| | accacttcagcctgaaagtgaacagcgtgacagccgccgataccgccgtgtactactg | |
| | cgccagattcgactacggcttccacgacgccttcgacatctggggccagggcacaatg | |
| | gtcacagtttctagcggaggcggaggatctggtggcggaggaagtggcggaggcggtt | |
| | ctgagattgtgatgacacagagccccgccactctgagccttagtcctggcgaaagagc | |
| | cacactgagctgcagagccagccagagcatcaccagcgattacctgagctggtatcag | |
| | cagaagcccggacaggctcccagactgctgatctatggcgcctctacaagagccaccg | |
| | gcattcccgcccgcttttctggctctggaagcggcaccgacttcaccctgaccatatc | |
| | tagcctgcagcctgaggacttcgtggtgtactattgccagcaggactacaacctgtac | |
| | accttcggccaggggaccaagctggaaatcaagagagagtctaaatacggaccgcctt | |
| | gtcctccttgtcccatgttctgggtgctcgtggtcgttggcggagtgctggcctgtta | |
| | cagcctgctggttaccgtggccttcatcatcttttgggtcaagcggggcagaaagaag | |
| | ctgctctacatcttcaagcagcccttcatgcggcccgtgcagaccacacaagaggaag | |
| | atggctgctcctgcagattccccgaggaagaagaaggcggctgcgagctgagagtgaa | |
| | gttcagcagatccgccgacgctccagcctatcagcagggccaaaaccagctgtacaac | |
| | gagctgaacctggggagaagagaagagtacgacgtgctggataagcggagaggcagag | |
| | atcctgaaatgggcggcaagcccagacggaagaatcctcaagagggcctgtataatga | |
| | gctgcagaaagacaagatggccgaggcctacagcgagatcggaatgaagggcgagcgc | |
| | agaagaggcaagggacacgatggactgtaccagggcctgagcaccgccaccaaggata | |
| | cctatgacgcactgcacatgcaggccctgccacctaga | |
| 158 | caggttcagctgcaagagtctggccctggcctggtcaagcctagcgaaacactgagcc | anti-ROR1 |
| | tgacctgtaccgtgtctggcggcagcatctccaactactactggtcctggatcagaca | CAR (nt) |
| | gcctgccggcaaaggcctggaatggatcggcagaatctacaccagcggcagcaccaac | |
| | tacaaccccagcctgaagtccagagtgaccatgagcgtggacaccagcaagaaccagt | |
| | tctccctgaagctgagcagcctgacagccgccgataccgccatctactactgtgcccg | |
| | gtactacgatatcctgaccggcttcttcgactactggggccagggaacactggtcaca | |
| | gtttctagcggaggcggaggatctggtggcggaggaagtggcggaggcggttctgtga | |
| | tttggatgacacagagccctagcctgctgagcgccagcacaggcgatagcgtgaccat | |
| | cagctgcagaatgagccaggacatcagcagctacctggcttggtatcagcagaagcct | |
| | ggcaaggcccctgaactgctgatctatgccgcttccagtctgcagagcggcgtgccat | |
| | ctagattttccggcagcggctctggcaccgacttcaccctgacaatcagctccctgca | |
| | gtccgaggacttcgccacctactattgccagcagtacgacagcttccctccaaccttt | |
| | ggccagggcaccaaggtggaattcaagcgcgagtctaaatacggaccgccttgtcctc | |
| | cttgtcccggccagccaagagagccccaggtttacacactgcctccaagccaagagga | |
| | aatgaccaagaatcaggtgtccctgacatgcctggtcaagggcttctacccctccgat | |
| | atcgccgtggaatgggagagcaatggccagcctgagaacaactacaagaccacacctc | |
| | ctgtgctggacagcgacggcagtttcttcctgtatagtagactcaccgtggataaatc | |
| | aagatggcaagagggcaacgtgttcagctgcagcgtgatgcacgaggccctgcacaac | |
| | cactacacccagaaaagcctgagcctgtctctgggcaagatgttctgggtgctcgtgg | |
| | tcgttggcggagtgctggcctgttacagcctgctggttaccgtggccttcatcatctt | |
| | ttgggtcaagcggggcagaaagaagctgctctacatcttcaagcagcccttcatgcgg | |
| | cccgtgcagaccacacaagaggaagatggctgctcctgcagattccccgaggaagaag | |
| | aaggcggctgcgagctgagagtgaagttcagcagatccgccgacgctccagcctatca | |
| | gcagggccaaaaccagctgtacaacgagctgaacctggggagaagagaagagtacgac | |
| | gtgctggataagcggagaggcagagatcctgaaatgggcggcaagcccagacggaaga | |
| | atcctcaagagggcctgtataatgagctgcagaaagacaagatggccgaggcctacag | |
| | cgagatcggaatgaagggcgagcgcagaagaggcaagggacacgatggactgtaccag | |
| | ggcctgagcaccgccaccaaggatacctatgacgcactgcacatgcaggccctgccac | |
| | ctaga | |
| 159 | caggtgcagctggttcaatctggcgccgaagtgaagaaaccaggcgcctctgtgaagg | anti-ROR1 |
| | tgtcctgcaaggccagcggctacacctttaccagctacggcatcagctgggtccgaca | CAR (nt) |
| | ggctcctggacaaggcttggaatggatgggctggatcagcgcctacaacggcaacacc | |
| | aaatacgcccagaaactgcagggcagagtgaccatgaccaccgacaccagcacaagca | |
| | ccgcctacatggaactgcggagcctgagatccgatgacaccgccgtgtactactgcgc | |
| | cagagatgaggacatcctgaccggctacaactactacggcatggacgtgtggggccag | |
| | ggcacaacagtgacagtttcttctggcggcggaggatctggcggaggtggaagcggag | |
| | gcggtggatctcaactggtgctgacacagtctcctagcgcctctgcttctctgggagc | |
| | cagcgtgaagctgacctgtacactgtctagcggccacagcagctacgccattgcttgg | |
| | catcagcagcagcccgagaagggccctagatacctgatgaagctgaacagcgacggca | |
| | gccactctaaaggcgacggcatccccgatagattcagcggcagttctagcggagccga | |
| | gcgctacctgacaatcagctctctgcaatccgaggacgaggccgactactactgtcag | |
| | acatggggcaccggcatcagagtgtttggcggaggcaccaagctgacagtgcttggag | |
| | agtctaaatacggaccgccttgtcctccttgtcccggccagccaagagagccccaggt | |
| | ttacacactgcctccaagccaagaggaaatgaccaagaatcaggtgtccctgacatgc | |
| | ctggtcaagggcttctacccctccgatatcgccgtggaatgggagagcaatggccagc | |
| | ctgagaacaactacaagaccacacctcctgtgctggacagcgacggcagtttcttcct | |
| | gtatagtagactcaccgtggataaatcaagatggcaagagggcaacgtgttcagctgc | |
| | agcgtgatgcacgaggccctgcacaaccactacacccagaaaagcctgagcctgtctc | |
| | tgggcaagatgttctgggtgctcgtggtcgttggcggagtgctggcctgttacagcct | |
| | gctggttaccgtggccttcatcatcttttgggtcaagcggggcagaaagaagctgctc | |
| | tacatcttcaagcagcccttcatgcggcccgtgcagaccacacaagaggaagatggct | |
| | gctcctgcagattccccgaggaagaagaaggcggctgcgagctgagagtgaagttcag | |
| | cagatccgccgacgctccagcctatcagcagggccaaaaccagctgtacaacgagctg | |
| | aacctggggagaagagaagagtacgacgtgctggataagcggagaggcagagatcctg | |
| | aaatgggcggcaagcccagacggaagaatcctcaagagggcctgtataatgagctgca | |
| | gaaagacaagatggccgaggcctacagcgagatcggaatgaagggcgagcgcagaaga | |
| | ggcaagggacacgatggactgtaccagggcctgagcaccgccaccaaggatacctatg | |
| | acgcactgcacatgcaggccctgccacctaga | |
| 160 | cagctccagctgcaagaatctggacctggcctggtcaagcccagcgagacactgtctc | anti-ROR1 |
| | tgacctgtacagtgtccggcggcagcatcaatagcaccacaagctactgggcctggat | CAR (nt) |
| | cagacagcctcctggcaaaggcctggaatggatcggcaccatcttctacagcggcaag | |
| | acctacaacaaccccagcctgaagtccagagtgaccatgagcgtggacaccagcaaga | |
| | accacttcagcctgaaagtgaacagcgtgacagccgccgataccgccgtgtactactg | |
| | cgccagattcgactacggcttccacgacgccttcgacatctggggccagggcacaatg | |
| | gtcacagtttctagcggaggcggaggatctggtggcggaggaagtggcggaggcggtt | |
| | ctgagattgtgatgacacagagccccgccactctgagccttagtcctggcgaaagagc | |
| | cacactgagctgcagagccagccagagcatcaccagcgattacctgagctggtatcag | |
| | cagaagcccggacaggctcccagactgctgatctatggcgcctctacaagagccaccg | |
| | gcattcccgcccgcttttctggctctggaagcggcaccgacttcaccctgaccatatc | |
| | tagcctgcagcctgaggacttcgtggtgtactattgccagcaggactacaacctgtac | |
| | accttcggccaggggaccaagctggaaatcaagagagagtctaaatacggaccgcctt | |
| | gtcctccttgtcccggccagccaagagagccccaggtttacacactgcctccaagcca | |
| | agaggaaatgaccaagaatcaggtgtccctgacatgcctggtcaagggcttctacccc | |
| | tccgatatcgccgtggaatgggagagcaatggccagcctgagaacaactacaagacca | |
| | cacctcctgtgctggacagcgacggcagtttcttcctgtatagtagactcaccgtgga | |
| | taaatcaagatggcaagagggcaacgtgttcagctgcagcgtgatgcacgaggccctg | |
| | cacaaccactacacccagaaaagcctgagcctgtctctgggcaagatgttctgggtgc | |
| | tcgtggtcgttggcggagtgctggcctgttacagcctgctggttaccgtggccttcat | |
| | catcttttgggtcaagcggggcagaaagaagctgctctacatcttcaagcagcccttc | |
| | atgcggcccgtgcagaccacacaagaggaagatggctgctcctgcagattccccgagg | |
| | aagaagaaggcggctgcgagctgagagtgaagttcagcagatccgccgacgctccagc | |
| | ctatcagcagggccaaaaccagctgtacaacgagctgaacctggggagaagagaagag | |
| | tacgacgtgctggataagcggagaggcagagatcctgaaatgggcggcaagcccagac | |
| | ggaagaatcctcaagagggcctgtataatgagctgcagaaagacaagatggccgaggc | |
| | ctacagcgagatcggaatgaagggcgagcgcagaagaggcaagggacacgatggactg | |
| | taccagggcctgagcaccgccaccaaggatacctatgacgcactgcacatgcaggccc | |
| | tgccacctaga | |
| 161 | caggttcagctgcttcagtctggcgccgaagtgaagaaacctggcgcctctgtgaagg | anti-ROR1 |
| | tgtcctgcaaggccagcggctacacctttaccagctacggcatcagctgggtccgaca | CAR (nt) |
| | ggctcctggacaaggcttggaatggatgggctggatcagcgcctacaccggcaatacc | |
| | agatacgcccagaaactgcagggcagagtgaccatgaccaccgacaccagcacaagca | |
| | ccgcctacatggaactgcggagcctgagatccgatgacaccgccgtgtactactgcgc | |
| | cagagaagaaggcgccaccaccgactacgactactacggcatggatgtgtggggccag | |
| | ggaacagccgtgacagtttcttctggtggcggaggatctggcggaggtggaagcggcg | |
| | gaggcggatctcaactggttctgacacagagcccaagcgcctctgcatctctgggagc | |
| | ttccgtgaagctgacctgcacactgtctagcggccacagcagctatgccattgcctgg | |
| | catcagcaacagcccgagaagggccctagatacctgatgaagctgaacagcgacggca | |
| | gccactctaaaggcgacggcatccccgatagattcagcggcagttctagcggagccga | |
| | gcgctacctgacaatcagctctctgcaatccgaggacgaggccgattactactgtcag | |
| | acatggggcaccggcatcagagtgtttggcggcggaacaaagctgaccgtgctgggcg | |
| | agtctaaatacggaccgccttgtcctccttgtcccggccagccaagagagccccaggt | |
| | ttacacactgcctccaagccaagaggaaatgaccaagaatcaggtgtccctgacatgc | |
| | ctggtcaagggcttctacccctccgatatcgccgtggaatgggagagcaatggccagc | |
| | ctgagaacaactacaagaccacacctcctgtgctggacagcgacggcagtttcttcct | |
| | gtatagtagactcaccgtggataaatcaagatggcaagagggcaacgtgttcagctgc | |
| | agcgtgatgcacgaggccctgcacaaccactacacccagaaaagcctgagcctgtctc | |
| | tgggcaagatgttctgggtgctcgtggtcgttggcggagtgctggcctgttacagcct | |
| | gctggttaccgtggccttcatcatcttttgggtcaagcggggcagaaagaagctgctc | |
| | tacatcttcaagcagcccttcatgcggcccgtgcagaccacacaagaggaagatggct | |
| | gctcctgcagattccccgaggaagaagaaggcggctgcgagctgagagtgaagttcag | |
| | cagatccgccgacgctccagcctatcagcagggccaaaaccagctgtacaacgagctg | |
| | aacctggggagaagagaagagtacgacgtgctggataagcggagaggcagagatcctg | |
| | aaatgggcggcaagcccagacggaagaatcctcaagagggcctgtataatgagctgca | |
| | gaaagacaagatggccgaggcctacagcgagatcggaatgaagggcgagcgcagaaga | |
| | ggcaagggacacgatggactgtaccagggcctgagcaccgccaccaaggatacctatg | |
| | acgcactgcacatgcaggccctgccacctaga | |
| 162 | SRGGGGSGGGGSGGGGSLEMA | linker |
| 163 | GSRGGGGSGGGGSGGGGSLEMA | linker |
| 164 | ggatctgcgatcgctccggtgcccgtcagtgggcagagcgcacatcgoccacagtccc | EF1alpha |
| | cgagaagttggggggaggggtcggcaattgaaccggtgcctagagaaggtggcgcggg | promoter with |
| | gtaaactgggaaagtgatgtcgtgtactggctccgcctttttcccgagggtgggggag | HTLV1 |
| | aaccgtatataagtgcagtagtcgccgtgaacgttctttttcgcaacgggtttgccgc | enhancer |
| | cagaacacagctgaagcttcgaggggctcgcatctctccttcacgcgcccgccgccct | |
| | acctgaggccgccatccacgccggttgagtcgcgttctgccgcctcccgcctgtggtg | |
| | cctcctgaactgcgtccgccgtctaggtaagtttaaagctcaggtcgagaccgggcct | |
| | ttgtccggcgctcccttggagcctacctagactcagccggctctccacgctttgcctg | |
| | accctgcttgctcaactctacgtctttgtttcgttttctgttctgcgccgttacagat | |
| | ccaagctgtgaccggcgcctac | |
| 165 | aatcaacctctggattacaaaatttgtgaaagattgactggtattcttaactatgttg | Woodchuck |
| | ctccttttacgctatgtggatacgctgctttaatgcctttgtatcatgctattgcttc | Hepatitis |
| | ccgtatggctttcattttctcctccttgtataaatcctggttgctgtctctttatgag | Virus (WHP) |
| | gagttgtggcccgttgtcaggcaacgtggcgtggtgtgcactgtgtttgctgacgcaa | Posttranscriptional |
| | cccccactggttggggcattgccaccacctgtcagctcctttccgggactttcgcttt | Regulatory |
| | ccccctccctattgccacggcggaactcatcgccgcctgccttgcccgctgctggaca | Element |
| | ggggctcggctgttgggcactgacaattccgtggtgttgtcggggaaatcatcgtcct | (WPRE) |
| | ttccttggctgctcgcctgtgttgccacctggattctgcgcgggacgtccttctgcta | |
| | cgtcccttcggccctcaatccagcggaccttccttcccgcggcctgctgccggctctg | |
| | cggcctcttccgcgtcttcgccttcgccctcagacgagtcggatctccctttgggccg | |
| | cctccccgc | |
| 166 | GSGEGRGSLLTCGDVEENPGP | T2A peptide |
| | | (aa) |
| 167 | GSGQCTNYALLKLAGDVESNPGP | E2A peptide |
| | | (aa) |
| 168 | GSGVKQTLNFDLLKLAGDVESNPGP | F2A peptide |
| | | (aa) |
| 169 | ctcgagggcggcggagagggcagaggaagtcttctaacatgcggtgacgtggaggaga | T2A peptide |
| | atcccggccctagg | (nt) |
| 170 | gagctggagcagccgccaccgccgccgccgagggagccccgggacggcagcccctggg | human ROR1 |
| | cgcagggtgcgctgttctcggagtccgacccagggcgactcacgcccactggtgcgac | (nt) |
| | ccggacagcctgggactgacccgccggcccaggcgaggctgcagccagagggctggga | GenBank: |
| | agggatcgcgctcgcggcatccagaggcggccaggcggaggcgagggagcaggttaga | M97675.1 |
| | gggacaaagagctttgcagacgtccccggcgtcctgcgagcgccagcggccgggacga | |
| | ggcggccgggagcccgggaagagcccgtggatgttctgcgcgcggcctgggagccgcc | |
| | gccgccgccgcctcagcgagaggaggaatgcaccggccgcgccgccgcgggacgcgcc | |
| | cgccgctcctggcgctgctggccgcgctgctgctggccgcacgcggggctgctgccca | |
| | agaaacagagctgtcagtcagtgctgaattagtgcctacctcatcatggaacatctca | |
| | agtgaactcaacaaagattcttacctgacccttgatgaaccaatgaataacatcacca | |
| | cgtctctgggccagacagcagaactgcactgcaaagtctctgggaatccacctcccac | |
| | catccgctggttcaaaaatgatgctcctgtggtccaggagccccggaggctctccttt | |
| | cggtccaccatctatggctctcggctgcggattagaaacctcgacaccacagacacag | |
| | gctacttccagtgcgtggcaacaaacggcaaggaggtggtttcttccactggagtctt | |
| | gtttgtcaagtttggcccccctcccactgcaagtccaggatactcagatgagtatgaa | |
| | gaagatggattctgtcagccatacagagggattgcatgtgcaagatttattggcaacc | |
| | gcaccgtctatatggagtctttgcacatgcaaggggaaatagaaaatcagatcacagc | |
| | tgccttcactatgattggcacttccagtcacttatctgataagtgttctcagttcgcc | |
| | attccttccctgtgccactatgccttcccgtactgcgatgaaacttcatccgtcccaa | |
| | agccccgtgacttgtgtcgcgatgaatgtgaaatcctggagaatgtcctgtgtcaaac | |
| | agagtacatttttgcaagatcaaatcccatgattctgatgaggctgaaactgccaaac | |
| | tgtgaagatctcccccagccagagagcccagaagctgcgaactgtatccggattggaa | |
| | ttcccatggcagatcctataaataaaaatcacaagtgttataacagcacaggtgtgga | |
| | ctaccgggggaccgtcagtgtgaccaaatcagggcgccagtgccagccatggaattcc | |
| | cagtatccccacacacacactttcaccgcccttcgtttcccagagctgaatggaggcc | |
| | attcctactgccgcaacccagggaatcaaaaggaagctccctggtgcttcaccttgga | |
| | tgaaaactttaagtctgatctgtgtgacatcccagcttgcgattcaaaggattccaag | |
| | gagaagaataaaatggaaatcctgtacatactagtgccaagtgtggccattcccctgg | |
| | ccattgctttactcttcttcttcatttgcgtctgtcggaataaccagaagtcatcgtc | |
| | ggcaccagtccagaggcaaccaaaacacgtcagaggtcaaaatgtggagatgtcaatg | |
| | ctgaatgcatataaacccaagagcaaggctaaagagctacctctttctgctgtacgct | |
| | ttatggaagaattgggtgagtgtgcctttggaaaaatctataaaggccatctctatct | |
| | cccaggcatggaccatgctcagctggttgctatcaagaccttgaaagactataacaac | |
| | ccccagcaatggatggaatttcaacaagaagcctccctaatggcagaactgcaccacc | |
| | ccaatattgtctgccttctaggtgccgtcactcaggaacaacctgtgtgcatgctttt | |
| | tgagtatattaatcagggggatctccatgagttcctcatcatgagatccccacactct | |
| | gatgttggctgcagcagtgatgaagatgggactgtgaaatccagcctggaccacggag | |
| | attttctgcacattgcaattcagattgcagctggcatggaatacctgtctagtcactt | |
| | ctttgtccacaaggaccttgcagctcgcaatattttaatcggagagcaacttcatgta | |
| | aagatttcagacttggggctttccagagaaatttactccgctgattactacagggtcc | |
| | agagtaagtccttgctgcccattcgctggatgccccctgaagccatcatgtatggcaa | |
| | attctcttctgattcagatatctggtcctttggggttgtcttgtgggagattttcagt | |
| | tttggactccagccatattatggattcagtaaccaggaagtgattgagatggtgagaa | |
| | aacggcagctcttaccatgctctgaagactgcccacccagaatgtacagcctcatgac | |
| | agagtgctggaatgagattccttctaggagaccaagatttaaagatattcacgtccgg | |
| | cttcggtcctgggagggactctcaagtcacacaagctctactactccttcagggggaa | |
| | atgccaccacacagacaacctccctcagtgccagcccagtgagtaatctcagtaaccc | |
| | cagatatcctaattacatgttcccgagccagggtattacaccacagggccagattgct | |
| | ggtttcattggcccgccaatacctcagaaccagcgattcattcccatcaatggatacc | |
| | caatacctcctggatatgcagcgtttccagctgcccactaccagccaacaggtcctcc | |
| | cagagtgattcagcactgcccacctcccaagagtcggtccccaagcagtgccagtggg | |
| | tcgactagcactggccatgtgactagcttgccctcatcaggatccaatcaggaagcaa | |
| | atattcctttactaccacacatgtcaattccaaatcatcctggtggaatgggtatcac | |
| | cgtttttggcaacaaatctcaaaaaccctacaaaattgactcaaagcaagcatcttta | |
| | ctaggagacgccaatattcatggacacaccgaatctatgatttctgcagaactgtaaa | |
| | atgcacaacttttgtaaatgtggtatacaggacaaactagacggccgtagaaaagatt | |
| | tatattcaaatgtttttattaaagtaaggttctcatttagcagacatcgcaacaagta | |
| | ccttctgtgaagtttcactgtgtcttaccaagcaggacagacactcggccag | |
| 171 | QETELSVSAELVPTSSWNTSSEIDKGSYLTLDEPMNNITTSLGQTAELHCKVSGNPPP | Mouse |
| | SIRWFKNDAPVVQEPRRISFRATNYGSRLRIRNLDTTDTGYFQCVATNGKKVVSTTGV | ROR1; |
| | LFVKFGPPPTASPGSSDEYEEDGFCQPYRGIACARFIGNRTVYMESLHMQGEIENQIT | GenBank No. |
| | AAFTMIGTSSHLSDKCSQFAIPSLCHYAFPYCDETSSVPKPRDLCRDECEVLENVLCQ | NP_038873 |
| | TEYIFARSNPMILMRLKLPNCEDLPQPESPEAANCIRIGIPMADPINKNHKCYNSTGV | |
| | DYRGTVSVTKSGRQCQPWNSQYPHTHSFTALRFPELNGGHSYCRNPGNQKEAPWCFTL | |
| | DENFKSDLCDIPACDSKDSKEKNKMEILYILVPSVAIPLATAFLEFFICVCRNNQKSS | |
| | SPPVQRQPKPVRGQNVEMSMLNAYKPKSKAKELPLSAVREMEELGECTEGKIYKGHLY | |
| | LPGMDHAQLVAIKTLKDYNNPQQWTEFQQEASLMAELHHPNIVCLLGAVTQEQPVCML | |
| | FEYMNQGDLHEFLIMRSPHSDVGCSSDEDGTVKSSLDHGDFLHIAIQIAAGMEYLSSH | |
| | FFVHKDLAARNILIGEQLHVKISDLGLSREIYSADYYRVQSKSSLPIRWMPPEAIMYG | |
| | KFSSDSDIWSFGVVLWEIFSFGLQPYYGFSNQEVIEMVRKRQLLPCSEDCPPRMYSLM | |
| | TECWNEIPSRRPRFKDIHVRLRSWEGLSSHTSSTTPSGGNATTQTTSLSASPVSNLSN | |
| | PRFPNYMFPSQGITPQGQIAGFIGPAIPQNQRFIPINGYPIPPGYAAFPAAHYQPAGP | |
| | PRVIQHCPPPKSRSPSSASGSTSTGHVASLPSSGSNQEANVPLLPHMSIPNHPGGMGI | |
| | TVFGNKSQKPYKIDSKQSSLLGDSHIHGHTESMISAEV | |
| 172 | QVQLQQSGAELVRPGASVTLSCKASGYTFSDYEMHWVIQTPVHGLEWIGAIDPETGGT | 2A2 V_{H} |
| | AYNQKFKGKAILTADKSSSTAYMELRSLTSEDSAVYYCTGYYDYDSFTYWGQGTLVTV | |
| | SA | |
| 173 | DIVMTQSQKIMSTTVGDRVS1TCKASQNVDAAVAWYQQKPGQSPKLLIYSASNRYTGV | 2A2 V_{L} |
| | PDRFTGSGSGTDFTLTISNMQSEDLADYFCQQYDIYPYTFGGGTKLEIK | |
| 174 | QVQLQESGPGLVKPSQTLSLTCTVSGYAFTAYNIHWVRQAPGQGLEWMGSFDPYDGGS | 99961 |
| | SYNQKFKDRLTISKDTSKNQVVLTMTNMDPVDTATYYCARGWYYFDYWGHGTLVTVSS | humanized |
| | | V_{H} |
| 175 | QVQLQESGPGLVKPSQTLSLTCTVSGYAFTAYNIHWVRQAPGQGLEWMGSFDPYDGGS | 99961 |
| | SYNQKFKDRLTISKDTSKNQVVLTMTNMDPVDTATYYCARGWYYFDYWGHGTLVTVSS | humanized |
| | | V_{H} |
| 176 | QVQLQESGPGLVKPSQTLSLTCTVSGYAFTAYNIHWIRQPPGKGLEWIGSFDPYDGGS | 99961 |
| | SYNQKFKDRLTISKDTSKNQVVLTMTNMDPVDTATYYCARGWYYFDYWGHGTLVTVSS | humanized |
| | | V_{H} |
| 177 | QVQLQESGPGLVKPSQTLSLTCTVSGYAFTAYNIHWIRQPPGKGLEWIGSFDPYDGGS | 99961 |
| | SYNQKFKDRLTISKDTSKNQVVLTMTNMDPVDTATYYCARGWYYFDYWGHGTLVTVSS | humanized |
| | | V_{H} |
| 178 | DIVMTQTPLSLPVTPGEPASISCRASKSISKYLAWYQQKPGQAPRLLIYSGSTLQSGT | 99961 |
| | PPRFSGSGYGTDFTLTINNIESEDAAYYFCQQHDESPYTFGEGTKVEIK | humanized |
| | | V_{L} |
| 179 | DVVMTQSPLSLPVTLGQPASISCRASKSISKYLAWYQQKPGKAPKLLIYSGSTLQSGT | 99961 |
| | PPRFSGSGYGTDFTLTINNIESEDAAYYFCQQHDESPYTFGEGTKVEIK | humanized |
| | | V_{L} |
| 180 | DIVMTQTPLSLPVTPGEPASISCRASKSISKYLAWYQQKPGQAPRLLIYSGSTLQSGT | 99961 |
| | PPRFSGSGYGTDFTLTINNIESEDAAYYFCQQHDESPYTFGEGTKVEIK | humanized |
| | | V_{L} |
| 181 | DVVMTQSPLSLPVTLGQPASISCRASKSISKYLAWYQQKPGKAPKLLIYSGSTLQSGT | 99961 |
| | PPRFSGSGYGTDFTLTINNIESEDAAYYFCQQHDESPYTFGEGTKVEIK | humanized |
| | | V_{L} |
| 182 | agagtcaagttttccaggtccgccgacgctccagcctaccagcaggggcagaaccagc | CD3-zeta |
| | tgtacaacgagctgaacctgggcagaagggaagagtacgacgtcctggataagcggag | derived |
| | aggccgggaccctgagatgggcggcaagcctcggcggaagaacccccaggaaggcctg | intracellular |
| | tataacgaactgcagaaagacaagatggccgaggcctacagcgagatcggcatgaagg | signaling |
| | gcgagcggaggcggggcaagggccacgacggcctgtatcagggcctgtccaccgccac | domain (nt) |
| | caaggatacctacgacgccctgcacatgcaggccctgcccccaagg | |
| 183 | aggagtaagaggagcaggctcctgcacagtgactacatgaacatgactccccgccgcc | CD28 |
| | ccgggcccacccgcaagcattaccagccctatgccccaccacgcgacttcgcagccta | endodomain |
| | tcgctcc | (nt) |
| 184 | QVQLQESGPGLVKPSETLSLTCTVSGGSISNYYWSWIRQPAGKGLEWIGRIYTSGSTN | anti-ROR1 |
| | YNPSLKSRVTMSVDTSKNQFSLKLSSLTAADTAIYYCARYYDILTGFEDYWGQGTLVT | CAR (aa) |
| | VSSGGGGSGGGGSGGGGSVIWMTQSPSLLSASTGDSVTISCRMSQDISSYLAWYQQKP | |
| | GKAPELLIYAASSLQSGVPSRFSGSGSGTDFTLTISSLQSEDFATYYCQQYDSFPPTF | |
| | GQGTKVEFKRESKYGPPCPPCPMFWVLVVVGGVLACYSLLVTVAFIIFWVKRGRKKLL | |
| | YIFKQPFMRPVQTTQEEDGCSCRFPEEEEGGCELRVKFSRSADAPAYQQGQNQLYNEL | |
| | NLGRREEYDVLDKRRGRDPEMGGKPRRKNPQEGLYNELQKDKMAEAYSEIGMKGERRR | |
| | GKGHDGLYQGLSTATKDTYDALHMQALPPR | |
| 185 | QLQLQESGPGLVKPSETLSLTCTVSGGSINSTTSYWAWIRQPPGKGLEWIGTIFYSGK | anti-ROR1 |
| | TYNNPSLKSRVTMSVDTSKNHFSLKVNSVTAADTAVYYCARFDYGEHDAFDIWGQGTM | CAR (aa) |
| | VTVSSGGGGSGGGGSGGGGSEIVMTQSPATLSLSPGERATLSCRASQSITSDYLSWYQ | |
| | QKPGQAPRLLIYGASTRATGIPARFSGSGSGTDFTLTISSLQPEDFVVYYCQQDYNLY | |
| | TFGQGTKLEIKRESKYGPPCPPCPMFWVLVVVGGVLACYSLLVTVAFIIFWVKRGRKK | |
| | LLYIFKQPFMRPVQTTQEEDGCSCRFPEEEEGGCELRVKFSRSADAPAYQQGQNQLYN | |
| | ELNLGRREEYDVLDKRRGRDPEMGGKPRRKNPQEGLYNELQKDKMAEAYSEIGMKGER | |
| | RRGKGHDGLYQGLSTATKDTYDALHMQALPPR | |
| 186 | QVQLQESGPGLVKPSETLSLTCTVSGGSISNYYWSWIRQPAGKGLEWIGRIYTSGSTN | anti-ROR1 |
| | YNPSLKSRVTMSVDTSKNQFSLKLSSLTAADTAIYYCARYYDILTGFEDYWGQGTLVT | CAR (aa) |
| | VSSGGGGSGGGGSGGGGSVIWMTQSPSLLSASTGDSVTISCRMSQDISSYLAWYQQKP | |
| | GKAPELLIYAASSLQSGVPSRFSGSGSGTDFTLTISSLQSEDFATYYCQQYDSFPPTF | |
| | GQGTKVEFKRESKYGPPCPPCPGQPREPQVYTLPPSQEEMTKNQVSLTCLVKGFYPSD | |
| | IAVEWESNGQPENNYKTTPPVLDSDGSFELYSRLTVDKSRWQEGNVFSCSVMHEALHN | |
| | HYTQKSLSLSLGKMFWVLVVVGGVLACYSLLVTVAFIIFWVKRGRKKLLYIFKQPFMR | |
| | PVQTTQEEDGCSCRFPEEEEGGCELRVKFSRSADAPAYQQGQNQLYNELNLGRREEYD | |
| | VLDKRRGRDPEMGGKPRRKNPQEGLYNELQKDKMAEAYSEIGMKGERRRGKGHDGLYQ | |
| | GLSTATKDTYDALHMQALPPR | |
| 187 | QVQLVQSGAEVKKPGASVKVSCKASGYTFTSYGISWVRQAPGQGLEWMGWISAYNGNT | anti-ROR1 |
| | KYAQKLQGRVTMTTDTSTSTAYMELRSLRSDDTAVYYCARDEDILTGYNYYGMDVWGQ | CAR (aa) |
| | GTTVTVSSGGGGSGGGGSGGGGSQLVLTQSPSASASLGASVKLTCTLSSGHSSYAIAW | |
| | HQQQPEKGPRYLMKLNSDGSHSKGDGIPDRFSGSSSGAERYLTISSLQSEDEADYYCQ | |
| | TWGTGIRVFGGGTKLTVLGESKYGPPCPPCPGQPREPQVYTLPPSQEEMTKNQVSLTC | |
| | LVKGFYPSDIAVEWESNGQPENNYKTTPPVLDSDGSFELYSRLTVDKSRWQEGNVFSC | |
| | SVMHEALHNHYTQKSLSLSLGKMFWVLVVVGGVLACYSLLVTVAFIIFWVKRGRKKLL | |
| | YIFKQPFMRPVQTTQEEDGCSCRFPEEEEGGCELRVKFSRSADAPAYQQGQNQLYNEL | |
| | NLGRREEYDVLDKRRGRDPEMGGKPRRKNPQEGLYNELQKDKMAEAYSEIGMKGERRR | |
| | GKGHDGLYQGLSTATKDTYDALHMQALPPR | |
| 188 | QLQLQESGPGLVKPSETLSLTCTVSGGSINSTTSYWAWIRQPPGKGLEWIGTIFYSGK | anti-ROR1 |
| | TYNNPSLKSRVTMSVDTSKNHFSLKVNSVTAADTAVYYCARFDYGEHDAFDIWGQGTM | CAR (aa) |
| | VTVSSGGGGSGGGGSGGGGSEIVMTQSPATLSLSPGERATLSCRASQSITSDYLSWYQ | |
| | QKPGQAPRLLIYGASTRATGIPARESGSGSGTDFTLTISSLQPEDFVVYYCQQDYNLY | |
| | TFGQGTKLEIKRESKYGPPCPPCPGQPREPQVYTLPPSQEEMTKNQVSLTCLVKGFYP | |
| | SDIAVEWESNGQPENNYKTTPPVLDSDGSFELYSRLTVDKSRWQEGNVFSCSVMHEAL | |
| | HNHYTQKSLSLSLGKMFWVLVVVGGVLACYSLLVTVAFIIFWVKRGRKKLLYIFKQPF | |
| | MRPVQTTQEEDGCSCRFPEEEEGGCELRVKFSRSADAPAYQQGQNQLYNELNLGRREE | |
| | YDVLDKRRGRDPEMGGKPRRKNPQEGLYNELQKDKMAEAYSEIGMKGERRRGKGHDGL | |
| | YQGLSTATKDTYDALHMQALPPR | |
| 189 | QVQLLQSGAEVKKPGASVKVSCKASGYTFTSYGISWVRQAPGQGLEWMGWISAYTGNT | anti-ROR1 |
| | RYAQKLQGRVTMTTDTSTSTAYMELRSLRSDDTAVYYCAREEGATTDYDYYGMDVWGQ | CAR (aa) |
| | GTAVTVSSGGGGSGGGGSGGGGSQLVLTQSPSASASLGASVKLTCTLSSGHSSYAIAW | |
| | HQQQPEKGPRYLMKLNSDGSHSKGDGIPDRFSGSSSGAERYLTISSLQSEDEADYYCQ | |
| | TWGTGIRVFGGGTKLTVLGESKYGPPCPPCPGQPREPQVYTLPPSQEEMTKNQVSLTC | |
| | LVKGFYPSDIAVEWESNGQPENNYKTTPPVLDSDGSFELYSRLTVDKSRWQEGNVFSC | |
| | SVMHEALHNHYTQKSLSLSLGKMFWVLVVVGGVLACYSLLVTVAFIIFWVKRGRKKLL | |
| | YIFKQPFMRPVQTTQEEDGCSCRFPEEEEGGCELRVKFSRSADAPAYQQGQNQLYNEL | |
| | NLGRREEYDVLDKRRGRDPEMGGKPRRKNPQEGLYNELQKDKMAEAYSEIGMKGERRR | |
| | GKGHDGLYQGLSTATKDTYDALHMQALPPR | |
| 190 | atgcctctgctgctgcttctgcctcttctttgggctggtgctctggct | CD33 signal |
| | | sequence (nt) |
| | | (O/SSE) |
| 191 | atgccgctgctgctactgctgcccctgctgtgggcaggggccctggct | CD33 signal |
| | | sequence (nt) |
| | | GenBank: |
| | | M23197.1 |
| 192 | gaatctaagtacggaccgccctgccccccttgccctatg | Spacer (IgG4 |
| | | hinge)(nt) |
| 193 | GAATCTAAGTACGGACCGCCTTGTCCTCCATGTCCTGGCCAGCCAAGAGAACCCCAGG | Hinge-C_{H}3 |
| | TGTACACACTGCCTCCAAGCCAAGAGGAAATGACCAAGAACCAGGTGTCCCTGACCTG | spacer (nt) |
| | CCTGGTCAAGGGCTTCTACCCTTCCGATATCGCCGTGGAATGGGAGAGCAATGGCCAG | |
| | CCTGAGAACAACTACAAGACCACACCTCCTGTGCTGGACAGCGACGGCTCATTCTTCC | |
| | TGTACAGCCGGCTGACCGTGGACAAGAGCAGATGGCAAGAGGGCAACGTGTTCAGCTG | |
| | CAGCGTGATGCACGAGGCCCTGCACAACCACTACACCCAGAAGTCTCTGAGCCTGAGC | |
| | CTGGGCAAG | |
| 194 | ESKYGPPCPPCPAPPVAGPSVFLFPPKPKDTLMISRTPEVTCVVVDVSQEDPEVQFNW | IgG4/IgG2 |
| | YVDGVEVHNAKTKPREEQFQSTYRVVSVLTVLHQDWLNGKEYKCKVSNKGLPSSIEKT | hinge- |
| | ISKAKGQPREPQVYTLPPSQEEMTKNQVSLTCLVKGFYPSDIAVEWESNGQPENNYKT | IgG2/IgG4 |
| | TPPVLDSDGSFFLYSRLTVDKSRWQEGNVFSCSVMHEALHNHYTQKSLSLSLGKM | C_{H}2-IgG4 |
| | | C_{H}3 spacer |
| | | (aa) |
| 195 | gaatctaagtacggaccgccctgccctccctgccctgctcctcctgtggctggaccaa | IgG4/IgG2 |
| | gcgtgttcctgtttccacctaagcctaaagataccctgatgatttcccgcacacctga | hinge- |
| | agtgacttgcgtggtcgtggacgtgagccaggaggatccagaagtgcagttcaactgg | IgG2/IgG4 |
| | tacgtggacggcgtggaagtccacaatgctaagactaaaccccgagaggaacagtttc | C_{H}2-IgG4 |
| | agtcaacttaccgggtcgtgagcgtgctgaccgtcctgcatcaggattggctgaacgg | C_{H}3 spacer |
| | gaaggagtataagtgcaaagtgtctaataagggactgcctagctccatcgagaaaaca | (nt) |
| | attagtaaggcaaaagggcagcctcgagaaccacaggtgtataccctgccccctagcc | |
| | aggaggaaatgaccaagaaccaggtgtccctgacatgtctggtcaaaggcttctatcc | |
| | aagtgacatcgccgtggagtgggaatcaaatgggcagcccgagaacaattacaagacc | |
| | acaccacccgtgctggactctgatggaagtttctttctgtattccaggctgaccgtgg | |
| | ataaatctcgctggcaggagggcaacgtgttctcttgcagtgtcatgcacgaagccct | |
| | gcacaatcattatacacagaagtcactgagcctgtccctgggcaaaatg | |
| 196 | gagtctaaatacggaccgccttgtcctccttgtcccgctcctcctgttgccggacctt | IgG4/IgG2 |
| | ccgtgttcctgtttcctccaaagcctaaggacaccctgatgatcagcaggacccctga | hinge- |
| | agtgacctgcgtggtggtggatgtgtcccaagaggatcccgaggtgcagttcaactgg | IgG2/IgG4 |
| | tatgtggacggcgtggaagtgcacaacgccaagaccaagcctagagaggaacagttcc | C_{H}2-IgG4 |
| | agagcacctacagagtggtgtccgtgctgacagtgctgcaccaggattggctgaacgg | C_{H}3 spacer |
| | caaagagtacaagtgcaaggtgtccaacaagggcctgcctagcagcatcgagaaaacc | (nt) O/SSE |
| | atctccaaggccaagggccagccaagagagccccaggtttacacactgcctccaagcc | |
| | aagaggaaatgaccaagaatcaggtgtccctgacatgcctggtcaagggcttctaccc | |
| | ctccgatatcgccgtggaatgggagagcaatggccagcctgagaacaactacaagacc | |
| | acacctcctgtgctggacagcgacggcagtttcttcctgtatagtagactcaccgtgg | |
| | ataaatcaagatggcaagagggcaacgtgttcagctgcagcgtgatgcacgaggccct | |
| | gcacaaccactacacccagaaaagcctgagcctgtctctgggcaagatg | |
| 197 | ttttgggtgctggtcgtggtcggaggggtgctggcctgttacagcctgctggtgacag | CD28 |
| | tcgctttcatcatcttctgggtg | transmembrane |
| | | domain (nt) |
| 198 | ttctgggtgctcgtggtcgttggcggagtgctggcctgttacagcctgctggttaccg | CD28 |
| | tggccttcatcatcttttgggtc | transmembrane |
| | | domain (nt) |
| | | (O/SSE) |
| 199 | FRSTIYGSRLRIRNL | ROR1 |
| | | epitope 1 |
| 200 | LSVSAELVPTSSW | ROR1 |
| | | epitope 2 |
| 201 | HCKVSGNPPPTIRW | ROR1 |
| | | epitope 3 |
| 202 | WFKNDAPVVQEPRRLSFRSTIYGSRL | ROR1 |
| | | epitope 4 |
| 203 | VSSTGVLFV | ROR1 |
| | | epitope 5 |
| 204 | LFVKFGPPPTASP | ROR1 |
| | | epitope 6 |
| 205 | DEYEEDGFCQP | ROR1 |
| | | epitope 7 |
| 206 | QPYRGIACARFIGN | ROR1 |
| | | epitope 8 |
| 207 | SQFAIPSLCHYAFP | ROR1 |
| | | epitope 9 |
| 208 | AFPYCDETSSVP | ROR1 |
| | | epitope 10 |
| 209 | NVLCQTEYIFARSNPMILMR | ROR1 |
| | | epitope 11 |
| 210 | LKLPNCEDLPQP | ROR1 |
| | | epitope 12 |
| 211 | PESPEAANCIRIGIP | ROR1 |
| | | epitope 13 |
| 212 | VDYRGTVSVTKSGR | ROR1 |
| | | epitope 14 |
| 213 | SQYPHTHTFTALRF | ROR1 |
| | | epitope 15 |
| 214 | DSKEKNKMEILYILV | ROR1 |
| | | epitope 16 |
| 215 | MHRPRRRGTRPPLLALLAALLLAARGAAAQETELSVSAELVPTSSWNISSELNKDSYLTLDEPM | human |
| | NNITTSLGQTAELHCKVSGNPPPTIRWFKNDAPVVQEPRRLSFRSTIYGSRLRIRNLDTTDTGY | (Homo |
| | FQCVATNGKEVVSSTGVLFVKFGPPPTASPGYSDEYEEDGFCQPYRGIACARFIGNRTVYMESL | sapiens) |
| | HMQGEIENQITAAFTMIGTSSHLSDKCSQFAIPSLCHYAFPYCDETSSVPKPRDLCRDECEILE | RORI |
| | NVLCQTEYIFARSNPMILMRLKLPNCEDLPQPESPEAANCIRIGIPMADPINKNHKCYNSTGVD | (Uniprot |
| | YRGTVSVTKSGRQCQPWNSQYPHTHTFTALRFPELNGGHSYCRNPGNQKEAPWCFTLDENFKSD | Q01973) |
| | LCDIPACDSKDSKEKNKMEILYILVPSVAIPLAIALLFFFICVCRNNQKSSSAPVQRQPKHVRG | |
| | QNVEMSMLNAYKPKSKAKELPLSAVRFMEELGECAFGKIYKGHLYLPGMDHAQLVAIKTLKDYN | |
| | NPQQWTEFQQEASLMAELHHPNIVCLLGAVTQEQPVCMLFEYINQGDLHEFLIMRSPHSDVGCS | |
| | SDEDGTVKSSLDHGDFLHIAIQIAAGMEYLSSHFFVHKDLAARNILIGEQLHVKISDLGLSREI | |
| | YSADYYRVQSKSLLPIRWMPPEAIMYGKFSSDSDIWSFGVVLWEIFSFGLQPYYGFSNQEVIEM | |
| | VRKRQLLPCSEDCPPRMYSLMTECWNEIPSRRPRFKDIHVRLRSWEGLSSHTSSTTPSGGNATT | |
| | QTTSLSASPVSNLSNPRYPNYMFPSQGITPQGQIAGFIGPPIPQNQRFIPINGYPIPPGYAAFP | |
| | AAHYQPTGPPRVIQHCPPPKSRSPSSASGSTSTGHVTSLPSSGSNQEANIPLLPHMSIPNHPGG | |
| | MGITVFGNKSQKPYKIDSKQASLLGDANIHGHTESMISAEL | |
| 216 | MHRPRRRGTRPPLLALLAALLLAARGAAAQETELSVSAELVPTSSWNISSELNKDSYLTLDEPM | Rhesus |
| | NNITTSLGQTAELHCKVSGNPPPTIRWFKNDAPVVQEPRRLSFRSTIYGSRLRIRNLDTTDTGY | macaque |
| | FQCVATNGKEVVSSTGVLFVKFGPPPTASPGYSDEYEEDGFCQPYRGIACARFIGNRTVYMESL | (Macaca |
| | HMQGEIENQITAAFTMIGTSSHLSDKCSQFAIPSLCHYAFPYCDETSSVPKPRDLCRDECEILE | mulatta) |
| | NVLCQTEYIFARSNPMILMRLKLPNCEDLPQPESPEAANCIRIGIPMADPINKNHKCYNSTGVD | ROR1 |
| | YRGTVSVTKSGRQCQPWNSQYPHTHTFTALRFPELNGGHSYCRNPGNQKEAPWCFTLDENFKSD | (Uniprot |
| | LCDIPACDSKDSKEKNKMEILYILVPSVAIPLAIALLFFFICVCRNNQKSSSPPVQRQPKHVRG | F6RUP2) |
| | QNVEMSMLNAYKPKSKAKELPLSAVRFMEELGECAFGKIYKGHLYLPGMDHAQLVAIKTLKDYN | |
| | NPQQWTEFQQEASLMAELHHPNIVCLLGAVTQEQPVCMLFEYMNQGDLHEFLIMRSPHSDVGCS | |
| | SDEDGTVKSSLDHGDFLHIAIQIAAGMEYLSSHFFVHKDLAARNILIGEQLHVKISDLGLSREI | |
| | YSADYYRVQSKSLLPIRWMPPEAIMYGKFSSDSDIWSFGVVLWEIFSFGLQPYYGFSNQEVIEM | |
| | VRKRQLLPCSEDCPPRMYSLMTECWNEIPSRRPRFKDIHVRLRSWEGLSSHTSSTTPSGGNATT | |
| | QTTSLSASPVSNLSNPRYPNYIFPSQGITPQGQIAGFIGPPIPQNQRFIPINGYPIPPGYAAFP | |
| | AAHYQPTGPPRVIQHCPPPKSRSPSSASGSTSTGHVTSLPSSGSNQEANIPLLPHMSIPNHPGG | |
| | MGITVFGNKSQKPYKIDAKQASLLGDANIHGHTESMISAEL | |
| 217 | MLRTAHKLLYLILPLSFSLPFFFFSETELSVSAELVPTSSWNISSELNKDSYLTLDEPMNNITT | cynomolgus |
| | SLGQTAELHCKVSGNPPPTIRWFKNDAPVVQEPRRLSFRSTIYGSRLRIRNLDTTDTGYFQCVA | macaque |
| | TNGKEVVSSTGVLFVKFGKDEYEEDGFCQPYRGIACARFIGNRTVYMESLHMQGEIENQITAAF | (macaca |
| | TMIGTSSHLSDKCSQFAIPSLCHYAFPYCDETSSVPKPRDLCRDECEILENVLCQTEYIFARSN | fasicularis) |
| | PMILMRLKLPNCEDLPQPESPEAANCIRIGIPMADPINKNHKCYNSTGVDYRGTVSVTKSGRQC | ROR1 |
| | QPWNSQYPHTHTFTALRFPELNGGHSYCRNPGNQKEAPWCFTLDENFKSDLCDIPACDSKDSKE | (Uniprot |
| | KNKMEILYILVPSVAIPLAIALLFFFICVCRNNQKSSSPPVQRQPKHVRGQNVEMSMLNAYKPK | A0A2K5WT |
| | SKAKELPLSAVRFMEELGECAFGKIYKGHLYLPGMDHAQLVAIKTLKDYNNPQQWTEFQQEASL | X7) |
| | MAELHHPNIVCLLGAVTQEQPVCMLFEYMNQGDLHEFLIMRSPHSDVGCSSDEDGTVKSSLDHG | |
| | DFLHIAIQIAAGMEYLSSHFFVHKDLAARNILIGEQLHVKISDLGLSREIYSADYYRVQSKSLL | |
| | PIRWMPPEAIMYGKFSSDSDIWSFGVVLWEIFSFGLQPYYGFSNQEVIEMVRKRQLLPCSEDCP | |
| | PRMYSLMTECWNEIPSRRPRFKDIHVRLRSWEGLSSHTSSTTPSGGNATTQTTSLSASPVSNLS | |
| | NPRYPNYIFPSQGITPQGQIAGFIGPPIPQNQRFIPINGYPIPPGYAAFPAAHYQPTGPPRVIQ | |
| | HCPPPKSRSPSSASGSTSTGHVTSLPSSGSNQEANIPLLPHMSIPNHPGGMGITVFGNKSQKPY | |
| | KIDAKQASLLGDANIHGHTESMISAEL | |
| 218 | SYLTLDEPMNNITTSLGQTAELHCKVSGNPPPTIRWFKNDAPVVQEPRRLSFRSTIYGSRLRIR | cynomolgus |
| | NLDTTDTGYFQCVATNGKEVVSSTGVLFVKFGPPPTASPGYSDEYEEDGFCQPYRGIACARFIG | macaque |
| | NRTVYMESLHMQGEIENQITAAFTMIGTSSHLSDKCSQFAIPSLCHYAFPYCDETSSVPKPRDL | (macaca |
| | CRDECEILENVLCQTEYIFARSNPMILMRLKLPNCEDLPQPESPEAANCIRIGIPMADPINKNH | fasicularis) |
| | KCYNSTGVDYRGTVSVTKSGRQCQPWNSQYPHTHTFTALRFPELNGGHSYCRNPGNQKEAPWCF | ROR1 |
| | TLDENFKSDLCDIPACDSKDSKEKNKMEILYILVPSVAIPLAIALLFFFICVCRNNQKSSSPPV | (Uniprot |
| | QRQPKHVRGQNVEMSMLNAYKPKSKAKELPLSAVRFMEELGECAFGKIYKGHLYLPGMDHAQLV | A0A2K5WT |
| | AIKTLKDYNNPQQWTEFQQEASLMAELHHPNIVCLLGAVTQEQPVCMLFEYMNQGDLHEFLIMR | X4) |
| | SPHSDVGCSSDEDGTVKSSLDHGDFLHIAIQIAAGMEYLSSHFFVHKDLAARNILIGEQLHVKI | |
| | SDLGLSREIYSADYYRVQSKSLLPIRWMPPEAIMYGKFSSDSDIWSFGVVLWEIFSFGLQPYYG | |
| | FSNQEVIEMVRKRQLLPCSEDCPPRMYSLMTECWNEIPSRRPRFKDIHVRLRSWEGLSSHTSST | |
| | TPSGGNATTQTTSLSASPVSNLSNPRYPNYIFPSQGITPQGQIAGFIGPPIPQNQRFIPINGYP | |
| | IPPGYAAFPAAHYQPTGPPRVIQHCPPPKSRSPSSASGSTSTGHVTSLPSSGSNQEANIPLLPH | |
| | MSIPNHPGGMGITVFGNKSQKPYKIDAKQASLLGDANIHGHTESMISAEL | |
| 219 | MHRPRRRGTRPPPLALLAALLLAARGADAQETELSVSAELVPTSSWNTSSEIDKGSYLTLDEPM | mouse (mus |
| | NNITTSLGQTAELHCKVSGNPPPSIRWFKNDAPVVQEPRRISFRATNYGSRLRIRNLDTTDTGY | musculus) |
| | FQCVATNGKKVVSTTGVLFVKFGPPPTASPGSSDEYEEDGFCQPYRGIACARFIGNRTVYMESL | ROR1 |
| | HMQGEIENQITAAFTMIGTSSHLSDKCSQFAIPSLCHYAFPYCDETSSVPKPRDLCRDECEVLE | (Uniprot |
| | NVLCQTEYIFARSNPMILMRLKLPNCEDLPQPESPEAANCIRIGIPMADPINKNHKCYNSTGVD | Q9Z139) |
| | YRGTVSVTKSGRQCQPWNSQYPHTHSFTALRFPELNGGHSYCRNPGNQKEAPWCFTLDENFKSD | |
| | LCDIPACDSKDSKEKNKMEILYILVPSVAIPLAIAFLFFFICVCRNNQKSSSPPVQRQPKPVRG | |
| | QNVEMSMLNAYKPKSKAKELPLSAVRFMEELGECTFGKIYKGHLYLPGMDHAQLVAIKTLKDYN | |
| | NPQQWTEFQQEASLMAELHHPNIVCLLGAVTQEQPVCMLFEYMNQGDLHEFLIMRSPHSDVGCS | |
| | SDEDGTVKSSLDHGDFLHIAIQIAAGMEYLSSHFFVHKDLAARNILIGEQLHVKISDLGLSREI | |
| | YSADYYRVQSKSSLPIRWMPPEAIMYGKFSSDSDIWSFGVVLWEIFSFGLQPYYGFSNQEVIEM | |
| | VRKRQLLPCSEDCPPRMYSLMTECWNEIPSRRPRFKDIHVRLRSWEGLSSHTSSTTPSGGNATT | |
| | QTTSLSASPVSNLSNPRFPNYMFPSQGITPQGQIAGFIGPAIPQNQRFIPINGYPIPPGYAAFP | |
| | AAHYQPAGPPRVIQHCPPPKSRSPSSASGSTSTGHVASLPSSGSNQEANVPLLPHMSIPNHPGG | |
| | MGITVFGNKSQKPYKIDSKQSSLLGDSHIHGHTESMISAEV | |

1. An anti-receptor tyrosine kinase-like orphan receptor 1 (ROR1) chimeric antigen receptor (CAR) comprising an extracellular antigen-binding domain comprising an anti-ROR1 antibody or antigen-binding fragment thereof comprising a heavy chain variable (V_{H}) region and a light chain variable (V_{L}) region, a transmembrane region and an intracellular signaling region, wherein:
the V_{H} region comprises a heavy chain complementarity determining region 1 (CDR-H1), a heavy chain complementarity determining region 2 (CDR-H2) and a heavy chain complementarity determining region 3 (CDR-H3) contained within SEQ ID NO:112, and the V_{L} region comprises a light chain complementarity determining region 1 (CDR-L1), a light chain complementarity determining region 2 (CDR-L2) and a light chain complementarity determining region 3 (CDR-L3) contained within SEQ ID NO:115;
the V_{H} region comprises a CDR-H1, a CDR-H2 and a CDR-H3 contained within SEQ ID NO:121, and the V_{L} region comprises a CDR-L1, a CDR-L2 and a CDR-L3 contained within SEQ ID NO: 124;
the V_{H} region comprises a CDR-H1, a CDR-H2 and a CDR-H3 contained within SEQ ID NO:103, and the V_{L} region comprises a CDR-L1, a CDR-L2 and a CDR-L3 contained within SEQ ID NO: 106; or
the V_{H} region comprises a CDR-H1, a CDR-H2 and a CDR-H3 contained within SEQ ID NO:130, and the V_{L} region comprises a CDR-L1, a CDR-L2 and a CDR-L3 contained within SEQ ID NO: 106.

2. The anti-ROR1 CAR of claim 1, wherein the extracellular antigen-binding domain comprises an amino acid sequence having at least 85% sequence identity to SEQ ID NO: 118, 127, 109 or 134.

3. The anti-ROR1 CAR of claim 1, wherein the extracellular antigen-binding domain comprises the amino acid sequence set forth in SEQ ID NO: 118, 127, 109 or 134.

4. The anti-ROR1 CAR of claim 1, further comprising a spacer between the extracellular antigen-binding domain and the transmembrane region.

5. The anti-ROR1 CAR of claim 4, wherein the spacer comprises at least a portion of a hinge region of an immunoglobulin or a variant thereof, comprising an amino acid sequence having at least 85%, identity to SEQ ID NO:1, 26, 27, 29, 31, 32, 33 or 135.

6. The anti-ROR1 CAR of claim 5, wherein the at least a portion of a hinge region comprises all or a portion of an IgG4 hinge region, comprising the amino acid sequence set forth in SEQ ID NO: 135.

7. The anti-ROR1 CAR of claim 1, wherein the transmembrane region comprises a transmembrane domain from CD28, comprising an amino acid sequence having at least 90% sequence identity to SEQ ID NO: 8.

8. The anti-ROR1 CAR of claim 1, wherein the intracellular signaling region comprises a cytoplasmic signaling domain of a CD3-zeta (CD3ζ chain, comprising an amino acid sequence having at least at 90% sequence identity to SEQ ID NO:13, 14 or 15.

9. The anti-ROR1 CAR of claim 1, wherein the intracellular signaling region further comprises a costimulatory signaling region between the transmembrane region and the intracellular signaling region.

10. The anti-ROR1 CAR of claim 9, wherein the costimulatory signaling region comprises an intracellular signaling domain of a T cell costimulatory molecule or a signaling portion thereof.

11. The anti-ROR1 CAR of claim 9, wherein the costimulatory signaling region comprises an intracellular signaling domain of CD28, 4-1BB, or ICOS.

12. The anti-ROR1 CAR of claim 9, wherein the costimulatory signaling region comprises an amino acid sequence having at least 90% sequence identity to SEQ ID NO: 12.

13. The anti-ROR1 CAR of claim 1, wherein the anti-ROR1 CAR comprises from its N to C terminus in order: the extracellular antigen-binding domain, the spacer, the transmembrane region and the intracellular signaling region.

14. The anti-ROR1 CAR of claim 1, wherein the anti-ROR1 CAR comprises a sequence that exhibits at least 85% sequence identity to the sequence set forth in SEQ ID NO: 184, 185, 186, 187, 188 or 189.

15. The anti-ROR1 CAR of claim 1, wherein the anti-ROR1 CAR comprises the sequence set forth in SEQ ID NO: 184.

16. A multispecific antibody, comprising a first antigen binding domain that binds receptor tyrosine kinase-like orphan receptor 1 (ROR1) and a second antigen-binding domain that binds to a second antigen, wherein the first antigen-binding domain comprises an anti-ROR1 antibody or antigen-binding fragment thereof comprising a heavy chain variable (V_{H}) region, and a light chain variable (V_{L}) region, wherein:
the V_{H} region comprises a heavy chain complementarity determining region 1 (CDR-H1), a heavy chain complementarity determining region 2 (CDR-H2) and a heavy chain complementarity determining region 3 (CDR-H3) contained within SEQ ID NO:112, and the V_{L} region comprises a light chain complementarity determining region 1 (CDR-L1), a light chain complementarity determining region 2 (CDR-L2) and a light chain complementarity determining region 3 (CDR-L3) contained within SEQ ID NO:115;
the V_{H} region comprises a CDR-H1, a CDR-H2 and a CDR-H3 contained within SEQ ID NO:121, and the V_{L} region comprises a CDR-L1, a CDR-L2 and a CDR-L3 contained within SEQ ID NO: 124;
the V_{H} region comprises a CDR-H1, a CDR-H2 and a CDR-H3 contained within SEQ ID NO:103, and the V_{L} region comprises a CDR-L1, a CDR-L2 and a CDR-L3 contained within SEQ ID NO: 106; or
the V_{H} region comprises a CDR-H1, a CDR-H2 and a CDR-H3 contained within SEQ ID NO:130, and the V_{L} region comprises a CDR-L1, a CDR-L2 and a CDR-L3 contained within SEQ ID NO: 106.

17. A polynucleotide comprising a nucleic acid encoding the anti-ROR1 CAR of claim 1.

18. A cell comprising the polynucleotide of claim 17.

19. A cell comprising the anti-ROR1 CAR of claim 1.

20. A method of detecting receptor tyrosine kinase-like orphan receptor 1 (ROR1) in a subject with an anti-ROR1 antibody or antigen-binding fragment thereof, comprising:
(a) labeling the anti-ROR1 antibody or antigen-binding fragment thereof with a detectable label to generate a labeled anti-ROR1 antibody or antigen-binding fragment;
(b) contacting a biological sample obtained from the subject with the labeled anti-ROR1 antibody or antigen-binding fragment under conditions that permit binding of the labeled anti-ROR1 antibody or antigen-binding fragment to the ROR1; and
(c) detecting the labeled anti-ROR1 antibody or antigen-binding fragment comprising performing a fluorescence polarization immunoassay (FPIA) or a fluorescence immunoassay (FIA), thereby detecting the ROR1.
